(19) United States
(12) Patent Application Publication
(10) Pub. No.: US 2025/0051344 A1
(43) Pub. Date: Feb. 13, 2025

(54) BIFUNCTIONAL SELECTIVE DEGRADERS OF SMARCA2 AND THERAPEUTIC USES THEREOF

(71) Applicant: Nurix Therapeutics, Inc., San Francisco, CA (US)

(72) Inventors: Katherine Keenan, San Francisco, CA (US); Rishab N. Iyer, San Francisco, CA (US); Tim R. Kane, San Francisco, CA (US); Jake C. Swartzel, San Francisco, CA (US)

(21) Appl. No.: 18/737,832

(22) Filed: Jun. 7, 2024

Related U.S. Application Data
(60) Provisional application No. 63/507,074, filed on Jun. 8, 2023.

(51) Int. Cl. C07D 487/10; A61K 31/501; A61K 31/506; A61K 31/5386; A61K 31/551; C07D 401/14; C07D 413/14; C07D 471/04; C07D 471/08; C07D 471/10; C07D 487/04; C07D 487/08; C07D 491/107; C07D 498/04; C07D 498/10; C07D 519/00

(52) U.S. Cl. CPC C07D 487/10; A61K 31/501; A61K 31/506; A61K 31/5386; A61K 31/551; C07D 401/14; C07D 413/14; C07D 471/04; C07D 471/08; C07D 471/10; C07D 487/04; C07D 487/08; C07D 491/107; C07D 498/04; C07D 498/10; C07D 519/00

(57) ABSTRACT

The present disclosure provides bifunctional compounds as selective SMARCA2 degraders via ubiquitin proteosome pathway, and their therapeutic use for treating cancers.

### CROSS-REFERENCE TO RELATED APPLICATIONS

This application claims the benefit of priority to U.S. Provisional Application No. 63/507,074, filed Jun. 8, 2023, which application is hereby incorporated by reference in its entirety.

### BACKGROUND

### Technical Field

The present invention provides novel bifunctional compounds for proteolytically degrading SMARCA2, and their therapeutic use for treating SMARCA2-mediated diseases, including cancers.

### Description of the Related Art

Mammalian SWItch/Sucrose Non-Fermentable (mSWI/SNF) chromatin remodeling complexes contain one of two mutually exclusive and highly homologous catalytic ATPase subunits, SMARCA2 or SMARCA4. Subunit mutations within the complexes are prevalent in cancer, and SMARCA4-mutated cancers show acute sensitivity to depletion of SMARCA2, making SMARCA2 an attractive oncological target.

Several groups have developed small molecule inhibitors of SMARCA2, but advancement of these molecules has been hindered by the requirement for selectivity over SMARCA4. See e.g., Wanior, M. et al J. Med. Chem. 63:14680 (2020). Kofink, C. et al Nature Commun. 13:5969 (2022). Two regions of the SMARCA2 protein with well-defined binding pockets relevant for small molecule inhibitor discovery are the catalytic ATPase domain and a conserved bromodomain that can interact with acetylated chromatin. High sequence homology between the ATPase domains of these proteins makes it challenging to develop inhibitors with sufficient selectivity. Known ATPase domain ligands are dual inhibitors of SMARCA2/4 and exhibit dose-limiting tolerability issues. Alternatively, while the bromodomain shows more potential for selectivity, its function is dispensable, and current ligands are ineffective at inhibiting cell proliferation.

While selective targeting of SMARCA2 over SMARCA4 has thus far proven challenging using inhibitors, repurposing the non-inhibitory bromodomain binders for use as targeted protein degraders holds promise. However, current degraders have shown poor selectivity in targeting SMARCA2 over SMARCA4. See e.g., WO 2021/083949, WO 2020/251971 and WO 2019/195201.

### BRIEF SUMMARY

Provided herein are bifunctional compounds exhibiting both potent inhibition and selectivity against SMARCA2 while sparing SMARCA4.

Provided herein are bifunctional compounds represented by Formula (I):

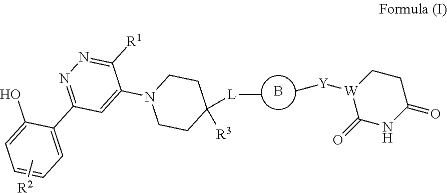

or a pharmaceutically acceptable salt thereof, wherein:
R¹ is hydrogen or C₁₋₆ alkyl optionally substituted with 1 to 3 R^{a};
R² is hydrogen, halo, hydroxyl, —O—R^{c}, C₁₋₆ alkyl optionally substituted with 1 to 3 R^{a};
R³ is hydrogen, —CN, C₁₋₆ alkyl, C₆₋₁₂ aryl, C₃₋₁₂ cycloalkyl, or 5-12 membered heteroaryl, each being optionally substituted with 1 to 3 R^{d}.
L comprises up to 8 linker segments represented by -L₁-L₂-L₃-L₄-L₅-L₆-L₇-L₈-, each L₁, L₂, L₃, L₄, L₅, L₆, L₇, or L₈, being independently:
   i) C₃₋₁₂ cycloalkyl optionally substituted with 1-3 R^{b};
   ii) C₆₋₁₂ aryl optionally substituted with 1-3 R^{b};
   iii) 4-12 membered heterocyclyl optionally substituted with 1-3 R^{b};
   iv) 5-12 membered heteroaryl optionally substituted with 1-3 R^{b};
   v) direct bond;
   vi) C₁₋₁₂ alkylene chain optionally substituted with 1-3 R^{b}; or
   vii) —(CH₂)ₘ—C(O)—, —(CH₂)ₘ—C(O)O—, —(CH₂)ₘ—O—, —(CH₂)ₘ—N(R^{c})—, —(CH₂)ₘ—S—, —(CH₂)ₘ—C(S)—, —(CH₂)ₘ—C(S)—O—, —(CH₂)ₘ—S(O)₂—, —(CH₂)ₘ—S(O)═N—, —(CH₂)ₘ—S(O)₂NH—, —(CH₂)ₘ—C(O)—N(R^{c})—, —C(O)—N(R^{c})—(CH₂)ₘ—, —CH₂)ₘ—O—C(O)—N(R^{c})—, —(CH₂)ₘ—O—C(O)—O—, or —NH—(CH₂)ₘ—C(O)—, wherein m is 0, 1, 2, 3, 4, 5 or 6;
each R^{a} is independently halo, or —O—R^{c};
each R^{b} is independently oxo, imino, sulfoximino, halo, nitro, —CN, C₁₋₆ alkyl, C₂₋₆ alkenyl, C₃₋₁₅ cycloalkyl, C₁₋₈ haloalkyl, C₆₋₁₂ aryl, 5-12 membered heteroaryl, 4-12 membered heterocyclyl, —O—R^{c}, —C(O)—R^{c}, —C(O)O—R^{c}, —C(O)—N(R^{c})(R^{c}), —N(R^{c})(R^{c}), —N(R^{c})C(O)—R^{c}, —N(R^{c})C(O)O—R^{c}, —N(R^{c})C(O)N(R^{c})(R^{c}), —N(R^{c})S(O)₂(R^{c}), —NR^{c}S(O)₂N(R^{c})(R^{c}), —N(R^{c})S(O)₂O(R^{c}), —OC(O)R^{c}, —OC(O)—N(R^{c})(R^{c}), —Si(R^{c})₃, —S—R^{c}, —S(O)R^{c}, —S(O)(NH)R^{c}, —S(O)₂R^{c} or —S(O)₂N(R^{c})(R^{c}), wherein each of C₁₋₆ alkyl, C₂₋₆ alkenyl, C₃₋₁₅ cycloalkyl, C₁₋₈ haloalkyl, C₆₋₁₂ aryl, 5-12 membered heteroaryl, and 4-12 membered heterocyclyl may be optionally substituted with 1 to 3 R^{d};
each R^{c} is independently hydrogen, C₁₋₆ alkyl, or C₁₋₆ haloalkyl;
each R^{d} is independently halo, —CN, —O—R^{c}, C₁₋₆ alkyl, or C₁₋₆ haloalkyl;
W is —C(R^{g})— or —N—;
Y is direct bond, C₁₋₄ alkylene chain, —C(O)—, —C(O)O—, —O—, —N(R^{g})—, —S—
—C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R^{j};
R^{g} is hydrogen or C₁₋₆ alkyl; and
each R^{j} is independently halo, —CN, —O—R^{c}, C₁₋₆ alkyl, C₆₋₁₂ aryl or C₁₋₆ haloalkyl.

In various specific embodiments, Y is direct bond, and the bifunctional compounds of Formula (I) are represented by Formula (II):

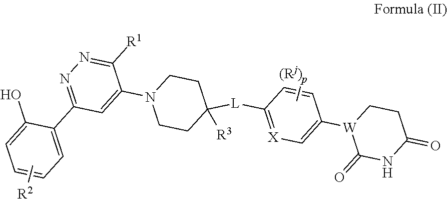

wherein,
R¹, R², R³, L, W are as defined above;
X is CH or N;
p is 0, 1, or 2; and
Rⱼ is halo or C₁₋₃ alkyl.

In various specific embodiments, the bifunctional compounds of Formula (II) are represented by Formula (IIa), (IIb), (IIc) or (IId):

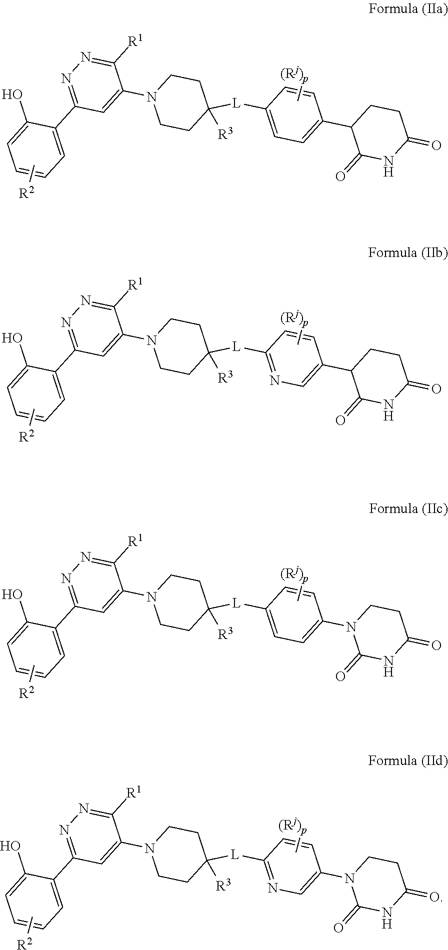

In more specific embodiments, R¹ and R² are each hydrogen, Y is direct bond, L₁ is —C(O)—, L′ is -L₂-L₃-L₄-L₅-L₆-L₇-L₈-, and the bifunctional compounds are represented by the structures of Formula (III) and substructures (IIIa), (IIIb), (IIIc) and (IIId):

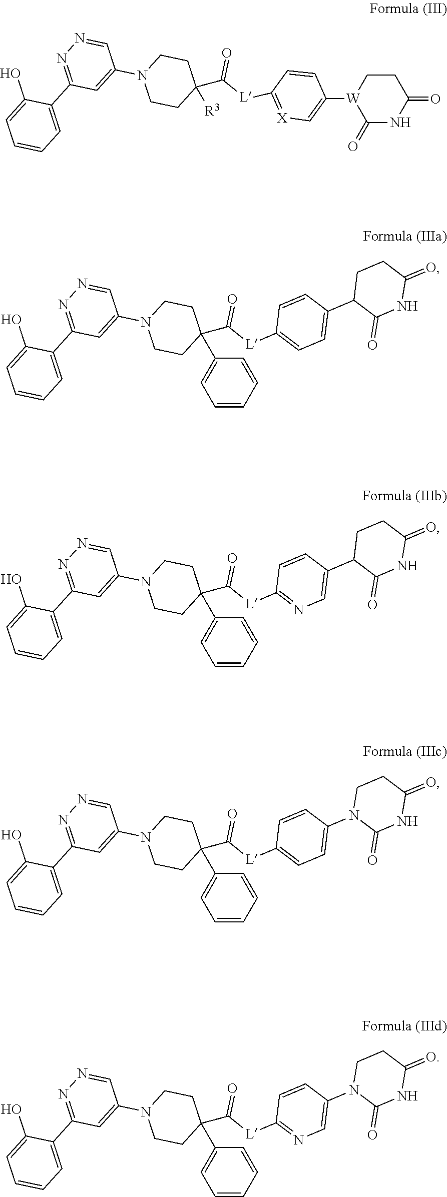

In even more specific embodiments, R¹ and R² are each hydrogen, Y is direct bond, L₁ is —C(O)—, L₈- is

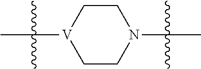

(V is CH or N) and L″ is -L₂-L₃-L₄-L₅-L₆-L₇- and the bifunctional compounds are represented by the structure of Formula (IV), (IVa), (IVb), and their respective substructures:

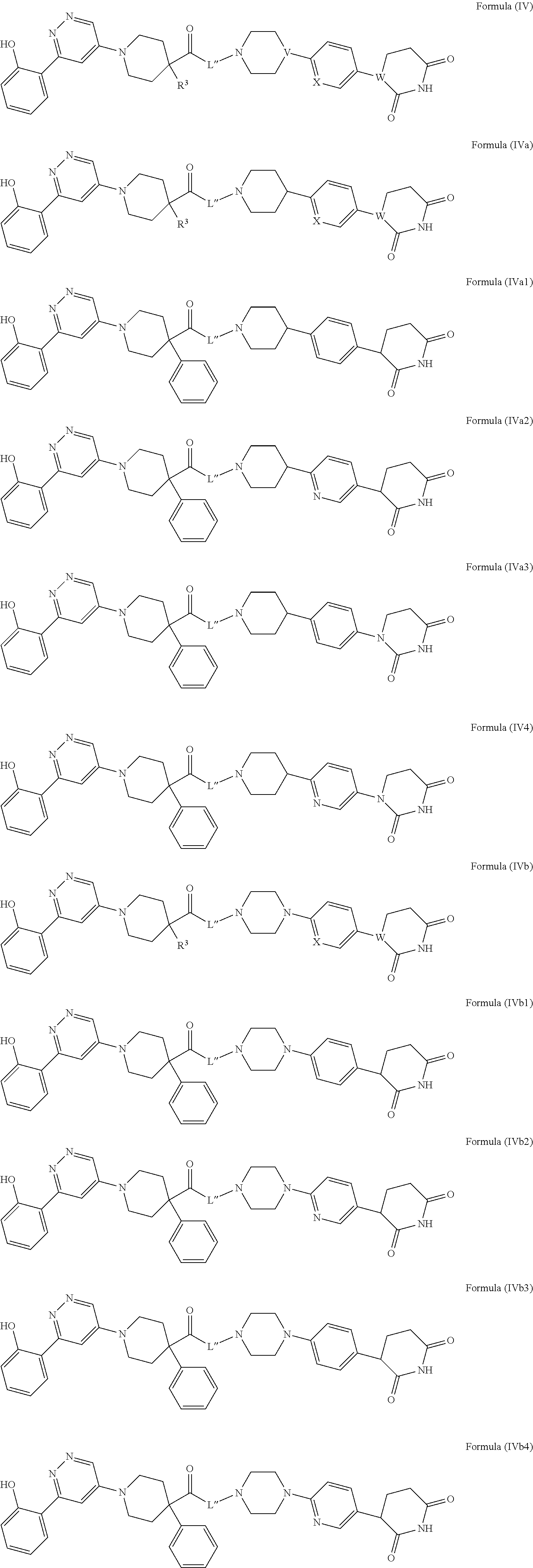

In various embodiments, each of the linker segments, namely, L₁, L₂, L₃, L₄, L₅, L₆, L₇, or L₈ is independently:
i) a bivalent ring moiety selected from the group consisting of

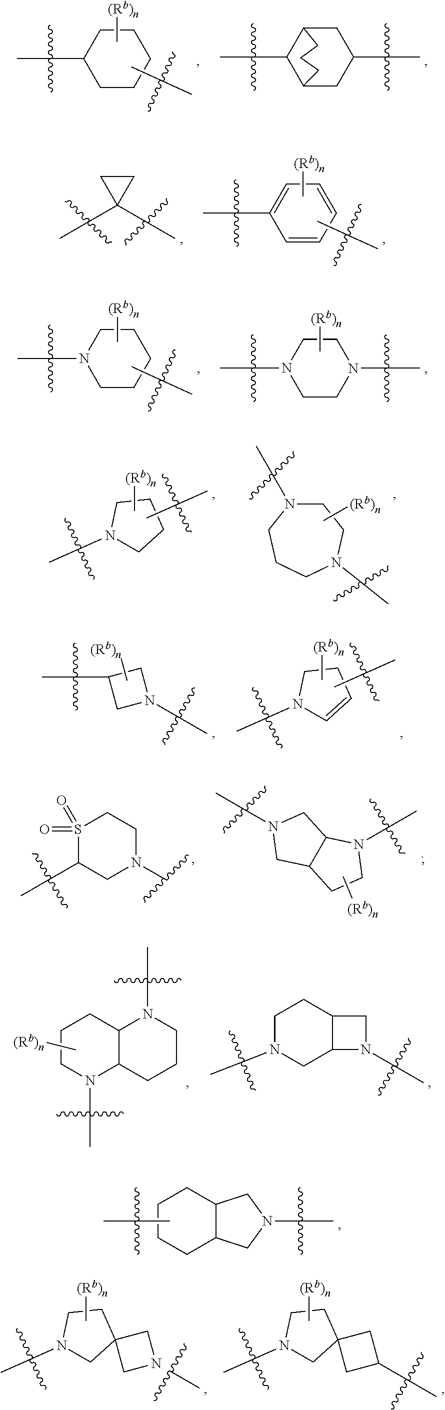

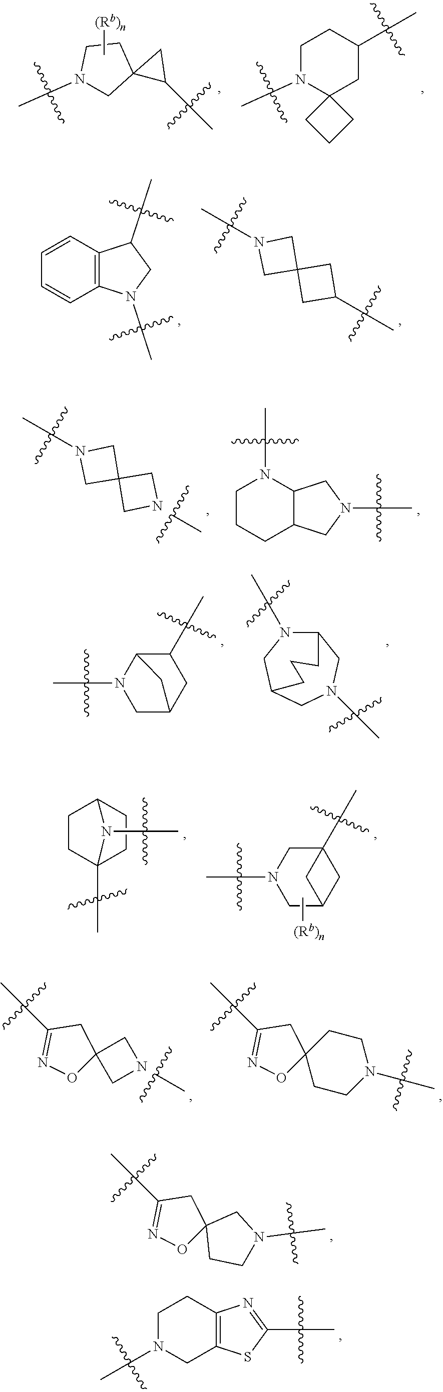

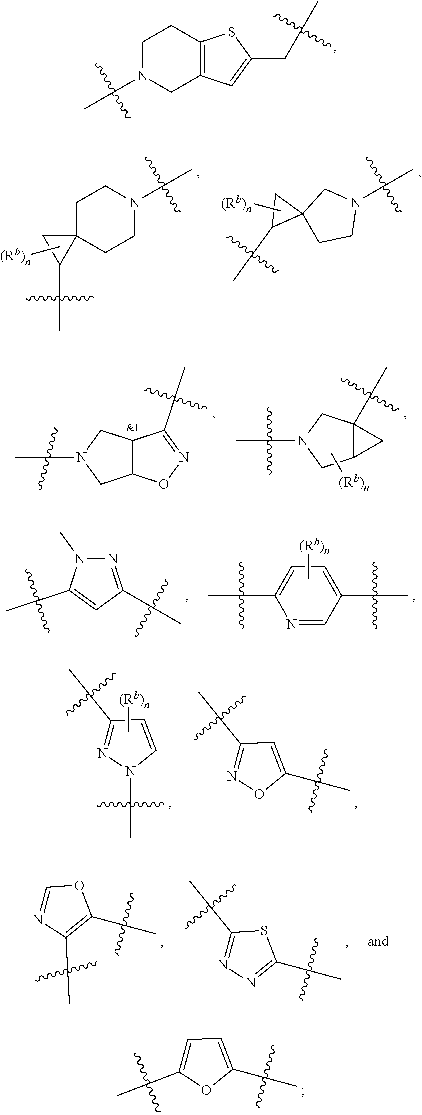

v) direct bond;
vi) C₁₋₆ alkylene chain; or
vii) —C(O)—, —O—, —C(O)—N(R^{c})—, —(CH₂)ₘ—C(O)—, or —NH—(CH₂)ₘ—C(O)—, wherein
m is 0, 1, 2 or 3;
wherein,
m is 0, 1, 2, or 3;
n is 0, 1, or 2;
R^{b} is halo, —CN, C₁₋₃ alkyl, or C₁₋₃ haloalkyl; and
R^{c} is hydrogen or C₁₋₃ alkyl.

Also provided herein is a pharmaceutical composition comprising a compound of Formulae (I), (II), (III) and (IV), (IA), or any one of the substructures or specific compounds of Examples 1-196 or a pharmaceutically acceptable salt thereof, and a pharmaceutically acceptable excipient or carrier.

### DETAILED DESCRIPTION

Specific degradation of SMARCA2 could be accomplished by using heterobifunctional small molecules to recruit SMARCA2, with high selectivity over SMARCA4, to a ubiquitin ligase and thus promoting ubiquitylation and proteasomal degradation of SMARCA2. Thus, provided herein are bifunctional compounds, each comprising a selective SMARCA2 binder, a covalent a linker moiety (L) and a ligase harness moiety (LHM) for targeting ubiquitin ligase. Preferably, the LHM targets cereblon (CRBN) proteins, which are substrate recognition subunits of two ubiquitously expressed and biologically important Cullin RING E3 ubiquitin ligase complexes. See, e.g., WO 2019/099926, WO 2020/023851, and U.S. Published Application No. 2019/0192668.

In addition to the compounds of Formula (I)-(IV) and their substructures described herein, various embodiments further provide a compound having a structure represented by Formula (IA):

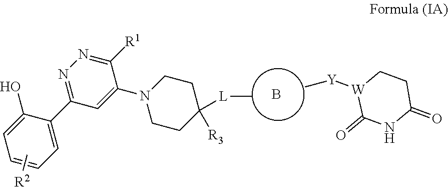

or a stereoisomer, pharmaceutically acceptable salt thereof, wherein:
R¹ is hydrogen or C₁₋₆ alkyl optionally substituted with 1 to 3 R^{a};
R² is hydrogen, halo, hydroxyl, —O—R^{c}, C₁₋₆ alkyl optionally substituted with 1 to 3 R^{a};
R³ is hydrogen, —CN, C₁₋₆ alkyl, C₆₋₁₂ aryl, C₃₋₁₂ cycloalkyl, 5-12 membered heteroaryl, each being optionally substituted with 1 to 3 R^{d}, or —O—R^{e};
L comprises up to 8 linker segments represented by -L₁-L₂-L₃-L₄-L₅-L₆-L₇-L₈-, each L₁, L₂, L₃, L₄, L₅, L₆, L₇, or L₈, being independently:
   i) C₃₋₁₂ cycloalkyl optionally substituted with 1-3 R^{b};
   ii) C₆₋₁₂ aryl optionally substituted with 1-3 R^{b};
   iii) 4-12 membered heterocyclyl optionally substituted with 1-3 R^{b};
   iv) 5-12 membered heteroaryl optionally substituted with 1-3 R^{b};
   v) direct bond;
   vi) C₁₋₁₂ alkylene chain optionally substituted with 1-3 R^{b}; or
   vii) —(CH₂)ₘ—C(O)—, —(CH₂)ₘ—C(O)O—, —(CH₂)ₘ—O—, —(CH₂)ₘ—N(R^{c})—,
   —(CH₂)ₘ—S—, —(CH₂)ₘ—C(S)—, —(CH₂)ₘ—C(S)—O—, —(CH₂)ₘ—S(O)₂—, —(CH₂)ₘ—S(O)═N—
   —(CH₂)ₘ—S(O)₂NH—, —(CH₂)ₘ—C(O)—N(R^{c})—, —C(O)—N(R^{c})—(CH₂)ₘ—, —(CH₂)ₘ O—C(O)—N(R^{c})—,
   —(CH₂)ₘ—O—C(O)—O—, or —NH—(CH₂)ₘ—C(O)—, wherein m is 0, 1, 2, 3, 4, 5 or 6;
each R^{a} is independently halo, or —O—R^{c}.
each R^{b} is independently oxo, imino, sulfoximino, halo, nitro, —CN, C₁₋₆ alkyl, C₂₋₆ alkenyl, C₃₋₁₅ cycloalkyl, C₁₋₈ haloalkyl, C₆₋₁₂ aryl, 5-12 membered heteroaryl, 4-12 membered heterocyclyl, —O—R^{c}, —C(O)—R^{c}, —C(O)O—R^{c}, —C(O)—N(R^{c})(R^{c}), —N(R^{c})(R^{c}), —N(R^{c})C(O)—R^{c}, —N(R^{c})C(O)O—R^{c}, —N(R^{c})C(O)N(R^{c})(R^{c}), —N(R^{c})S(O)₂(R^{c}), —NR^{c}S(O)₂N(R^{c})(R^{c}), —N(R^{c})S(O)₂O(R^{c}), —OC(O)R^{c}, —OC(O)—N(R^{c})(R^{c}), —Si(R^{c})₃, —S—R^{c}, —S(O)R^{c}, —S(O)(NH)R^{c}, —S(O)₂R^{c} or —S(O)₂N(R^{c})(R^{c}), wherein each of C₁₋₆ alkyl, C₂₋₆ alkenyl, C₃₋₁₅ cycloalkyl, C₁₋₈ haloalkyl, C₆₋₁₂ aryl, 5-12 membered heteroaryl, and 4-12 membered heterocyclyl may be optionally substituted with 1 to 3 R^{d};
each R^{c} is independently hydrogen, C₁₋₆ alkyl, C₃₋₆ cycloalkyl, C₆₋₁₂ aryl or C₁₋₆ haloalkyl, wherein C₁₋₆ alkyl is optionally substituted with C₁₋₃ alkoxy;
each R^{d} is independently halo, —CN, —O—R^{c}, C₁₋₆ alkyl, C₆₋₁₂ aryl or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl or halo;
W is —C(R^{g})— or —N—;
Y is direct bond, C₁₋₄ alkylene chain, —C(O)—, —C(O)O—, —O—, —N(R^{g})—, —S—
C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R^{j};
R^{g} is hydrogen or C₁₋₆ alkyl; and
each R^{j} is independently halo, oxo, —CN, —O—R^{c}, C₁₋₆ alkyl, or C₁₋₆ haloalkyl.

In more specific embodiments, each L₁, L₂, L₃, L₄, L₅, L₆, L₇, or L₈ is independently:
i) a bivalent ring moiety selected from the group consisting of:

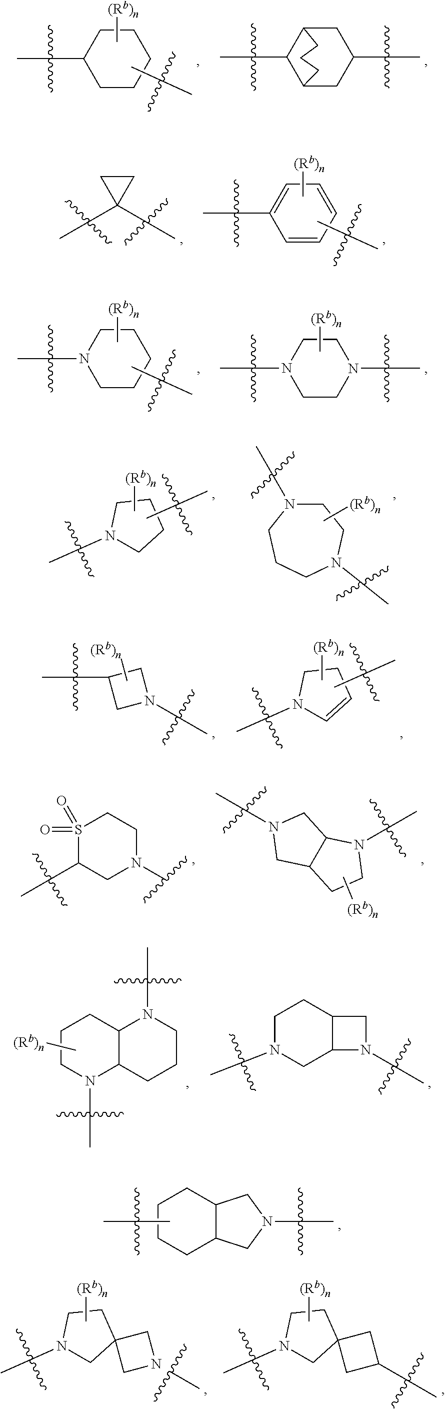

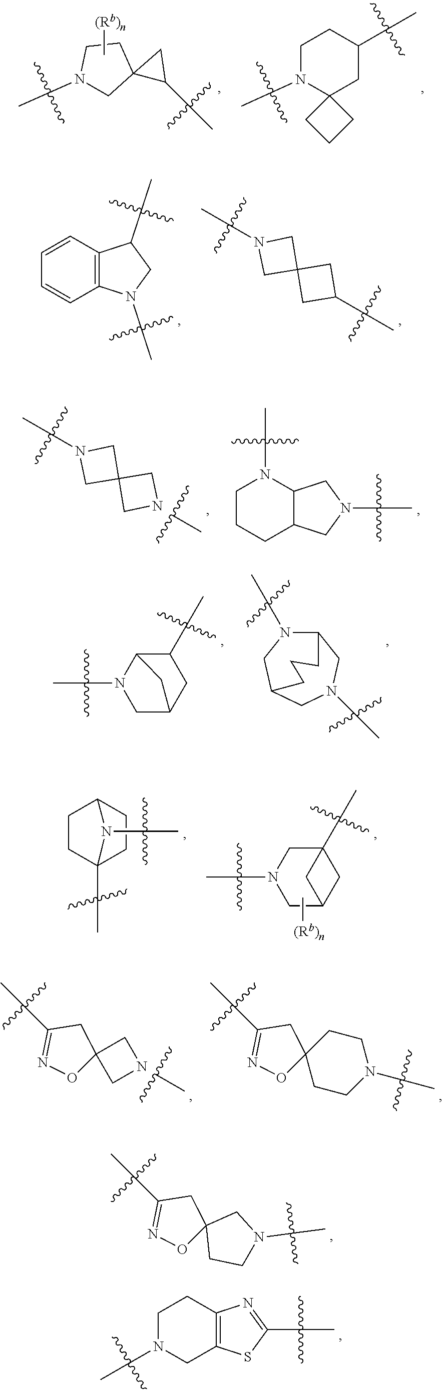

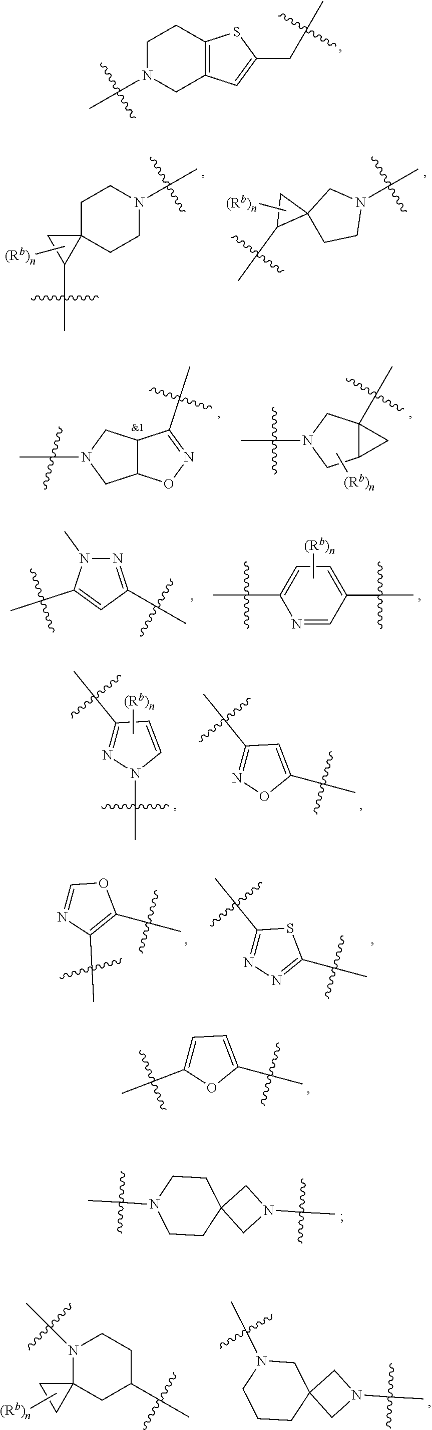

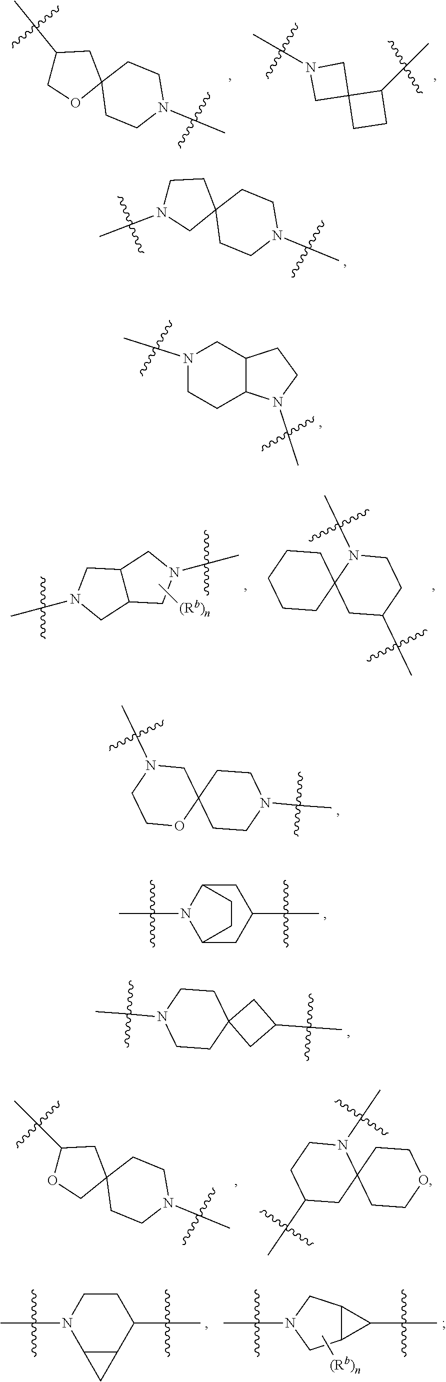

ii) direct bond;
iii) C₁₋₆ alkylene chain; or
iv) —C(O)—, —O—, —(CH₂)ₘ—C(O)—N(R^{c})—, —(CH₂)ₘ—N(R^{c})—, —C(O)—N(R^{c})—(CH₂)ₘ—, or —NH—(CH₂)ₘ—C(O)—, —(CH₂)ₘ—S(O)₂NH—, m being 0, 1, 2 or 3;
wherein,
n is 0, 1, or 2;
R^{b} is halo, —O—R^{c}, —CN, C₁₋₆ alkyl, or C₁₋₆ haloalkyl; and
R^{c} is hydrogen, C₁₋₆ alkyl, C₃₋₆ cycloalkyl, phenyl, or C₁₋₆ haloalkyl, wherein C₁₋₆ alkyl is optionally substituted with C₁₋₃ alkoxy.

In other more specific embodiments, Y is direct bond, —NHC(O)—, or —NH—; and
B ring is:

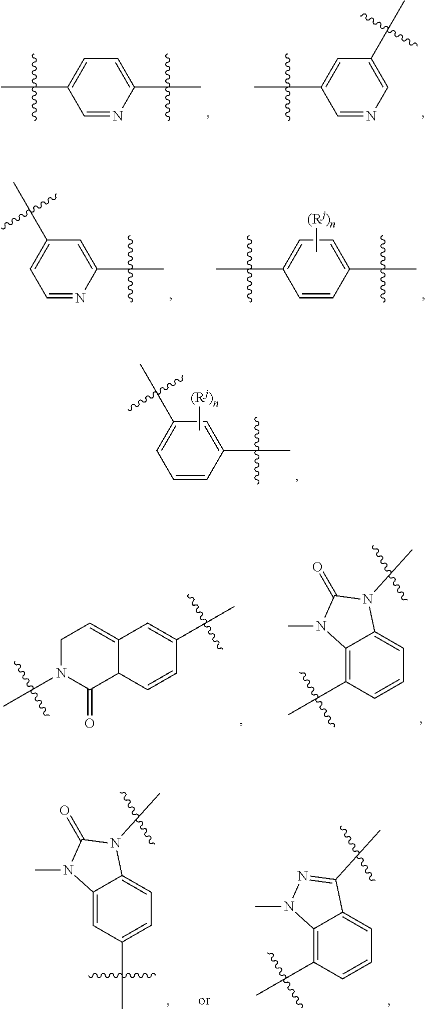

wherein n is 0, 1 or 2, R^{j} is halo, —CN, —O—R^{c}, C₁₋₆ alkyl, or C₁₋₆ haloalkyl.

In various embodiments, R³ is phenyl optionally substituted with one or more C₁₋₆ alkyl, C₁₋₆ alkoxy, or halo; benzyl optionally substituted with one or more C₁₋₆ alkyl or halo; pyridinyl optionally substituted with one or more C₁₋₆ alkyl; CN; C₃₋₆ cyclopropyl; pyrazolyl optionally substituted with one or more C₁₋₆ alkyl; tetrahydropyranyl; 1,2-oxazolyl optionally substituted with one or more C₁₋₆ alkyl; pyrazolo[1,5-a]pyridinyl; or —O—R^{e}, wherein R^{e} is phenyl optionally substituted with C₁₋₆ alkyl or halo, C₃₋₆ cycloalkyl, C₁₋₆ alkyl, or pyrazolyl optionally substituted with C₁₋₆ alkyl.

In even more specific embodiments, R³ is phenyl, benzyl, 2-pyridinyl, 3-pyridinyl, 4-pyridinyl, CN, cyclopropyl, 1-methyl-1H-pyrazol-3yl, 3-methylphenyl, 2-methylphenyl, 3-chlorophenyl, 3,4-dichlorophenyl, 2-methoxyphenyl, 3-methoxyphenyl, 2-fluorophenyl, 4-fluorophenyl, tetrahydropyran-4yl, 3-methyl-1H-pyrazol-1yl, 4-methyl-1H-pyrazol-1yl, 5-methyl-1H-pyrazol-1yl, 1-methyl-1H-pyrazol-3yl, 1-methyl-1H-pyrazol-4yl, 1-methyl-1H-pyrazol-5yl, pyrazolo[1,5-a]pyridin-2-yl, 5-methyl-1,2-oxazol-3-yl, 5-isopropyl-1,2-oxazol-3-yl, 3-methyl-1,2-oxazol-5-yl, or —O—R^{e}, wherein R^{e} is phenyl, 2-chlorophenyl, 2-methylphenyl, cyclopropyl, cyclohexyl, methyl, or 1-methyl-1H-pyrazol-3yl.

In certain embodiments, R¹ is hydrogen, R² is hydrogen, Y is direct bond, R³ is —O—R^{e}, W is CH, and the compound has a structure represented by Formula (IA1):

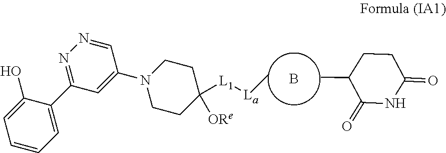

wherein,
B ring is

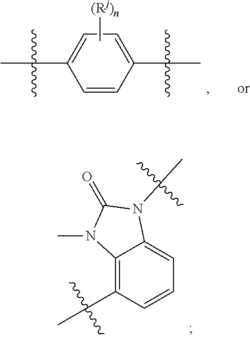

n is 0, 1 or 2;
R^{j} is halo, —CN, —O—R^{e}, C₁₋₆ alkyl, or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl or halo;
R^{c} is hydrogen or C₁₋₃ alkyl;
L₁ is —C(O)—, or —C(O)N(R^{c})—; and

Lₐ is

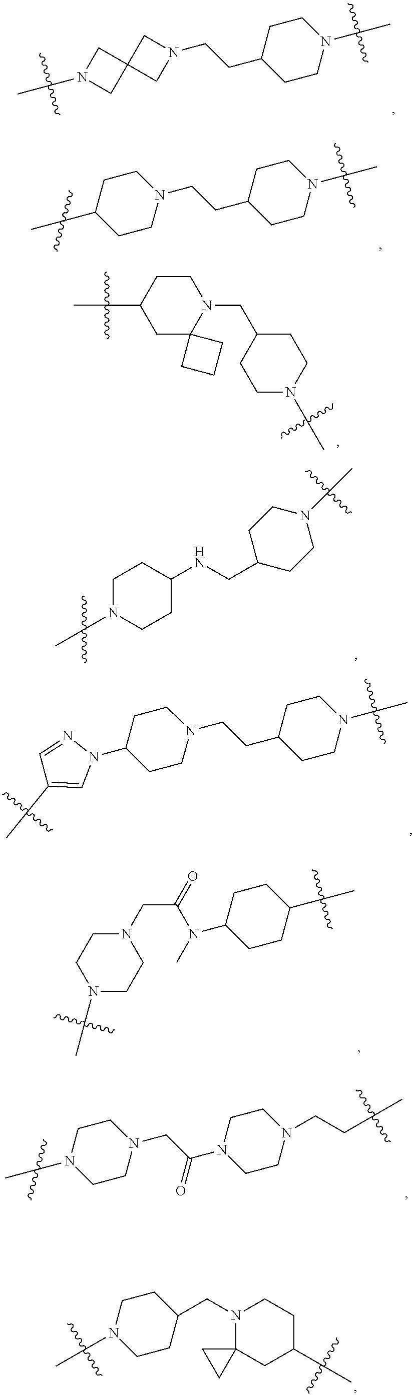

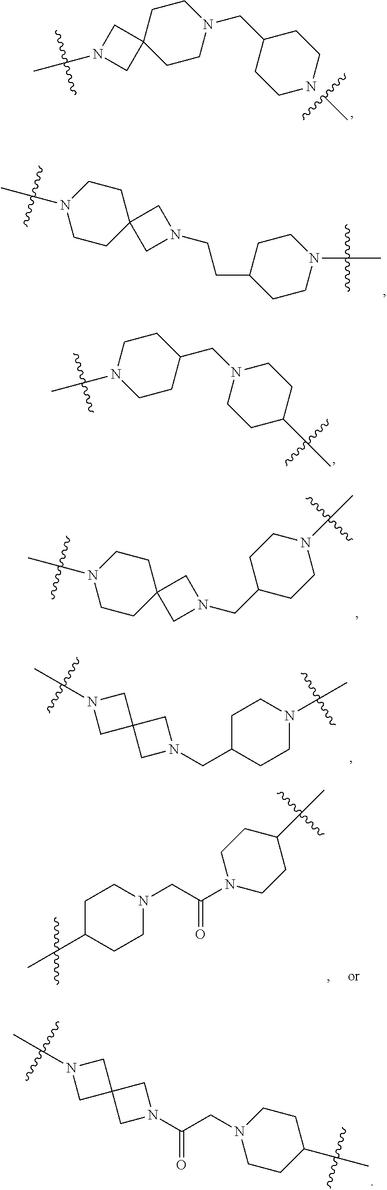

In more specific embodiments of Formula (IA1), B is

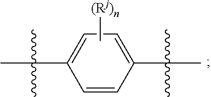

n is 0 or 1; R^{j} is fluoro or chloro; R^{e} is C₁₋₆ alkyl, C₃₋₁₂ cycloalkyl, C₆₋₁₂ aryl, 5-12 membered heteroaryl, each being optionally substituted with C₁₋₆ alkyl or halo; L¹ is —C(O)N(R^{c})— where R^{c} is methyl or ethyl; and Lₐ is

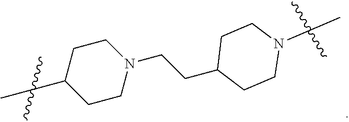 [SMILES: *C1CCN(CCC2CCN(*)CC2)CC1]

In various embodiments, R^{e} is C₁₋₃ alkyl.

In more specific embodiments, R^{e} is methyl, phenyl, cyclopropyl, cyclohexyl, 1-methyl-1H-pyrazol-3yl or phenyl substituted with chloro or methyl.

In other more specific embodiments of Formula (IA1), B ring is

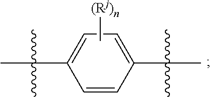

n is 0, 1 or 2; R^{j} is fluoro or chloro; R^{e} is methyl, ethyl, or propyl; R^{c} is hydrogen, methyl, or ethyl; L₁ is —C(O)—, or —C(O)N(R^{e})—; and

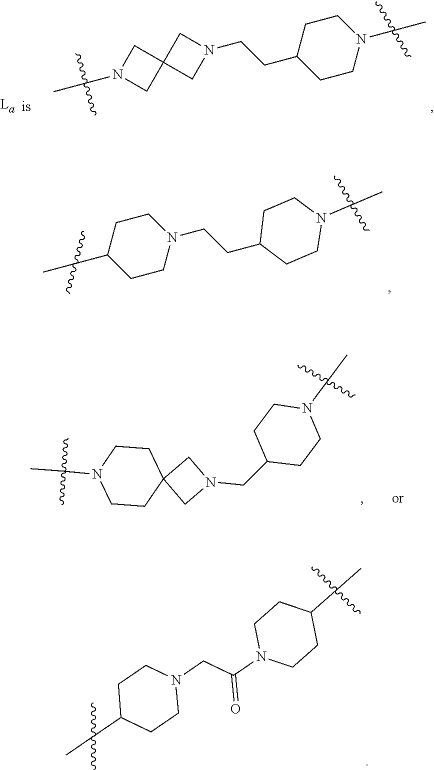 [SMILES: *N1CCC(CCN2CC3(CN(*)C3)C2)CC1 ; *C1CCN(CCC2CCN(*)CC2)CC1 ; *N1CCC(CN2CC3(CCN(*)CC3)C2)CC1 ; *C1CCN(CC(=O)N2CCC(*)CC2)CC1]

In certain embodiments, R¹ and R² are hydrogen, W is CH, and the compound has a structure represented by Formula (IA2):

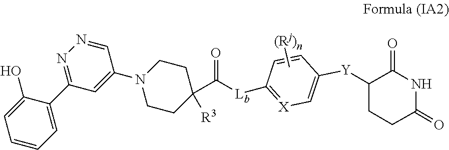

wherein,
n is 0, 1, or 2;
Y is direct bond, —NHC(O)—, or —NH—;
X is N or CH;
Rj is C₁₋₃alkyl or halo;
R³ is C₆₋₁₂ aryl, C₃₋₁₂ cycloalkyl, 5-12 membered heteroaryl, each being optionally substituted with 1 to 3 R^{d}, or —O—R^{e};
R^{c} is hydrogen or C₁₋₆ methyl;
each R^{d} is independently halo, —CN, —O—R^{c}, C₁₋₆ alkyl, C₆₋₁₂ aryl or C₁₋₆ haloalkyl; and
R^{e} is C₁₋₆ alkyl, C₃₋₁₂ cycloalkyl, C₆₋₁₂ aryl, 5-12 membered heteroaryl, each being optionally substituted with C₁₋₆ alkyl or halo; and
L_{b} is

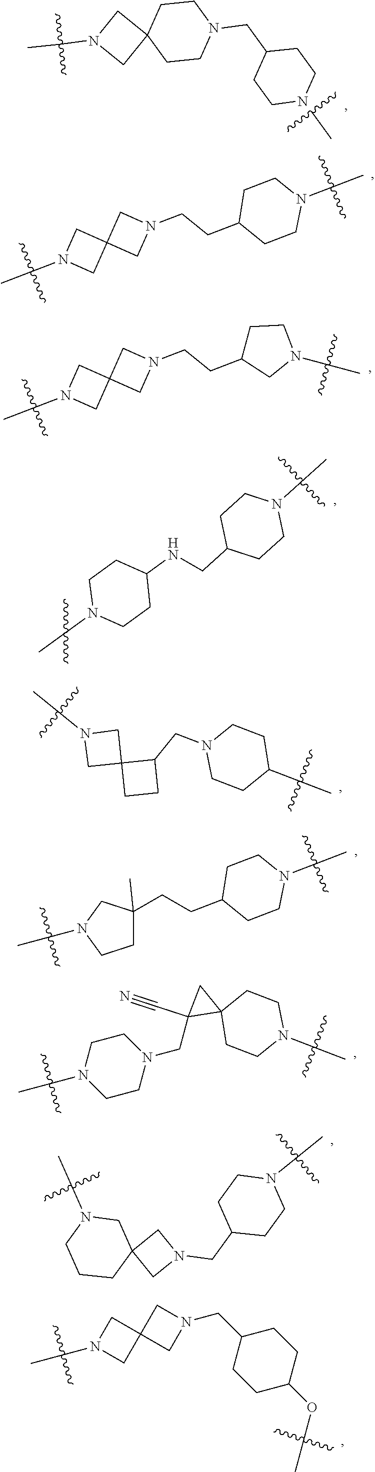

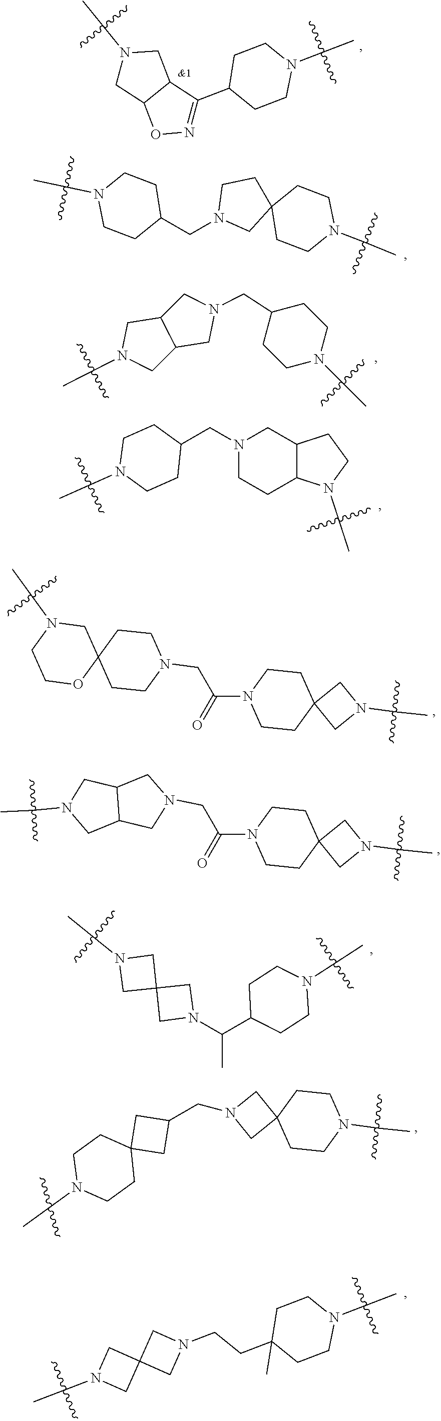

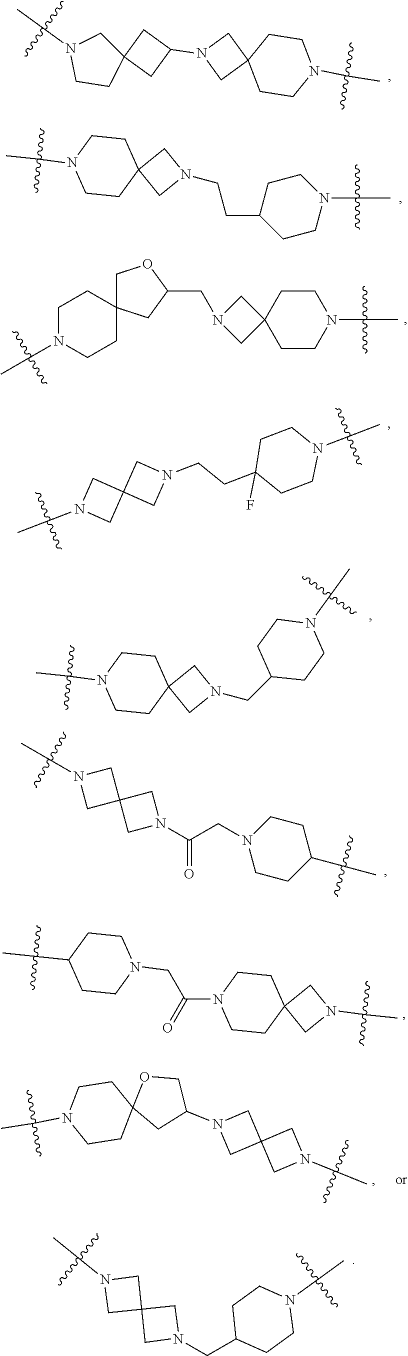

In more specific embodiments of Formula (IA2), R³ is phenyl, 2-fluorophenyl, 5-methyl-1,2-oxazol-5-yl, 5-isopropyl-1,2-oxazol-5-yl, pyrazolo[1,5-a]pyridinyl, —O—R^{e}, wherein R^{e} is phenyl, 2-chlorophenyl, 2-methylphenyl, 1-methyl-1H-pyrazol-3yl, cyclohexyl, or methyl.

In certain embodiments, R¹ and R² are hydrogen, W is CH, and the compound has a structure represented by Formula (IA3):

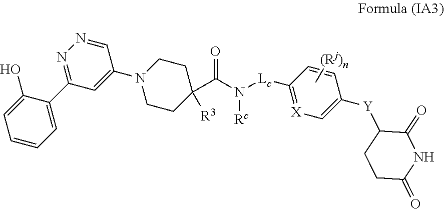

wherein,
n is 0, 1, or 2;
Y is direct bond, —NHC(O)—, or —NH—;
X is N or CH;
R³ is C₆₋₁₂ aryl, C₃₋₁₂ cycloalkyl, 5-12 membered heteroaryl, each being optionally substituted with 1 to 3 R^{d}, or —O—R^{e};
R^{c} is hydrogen, C₁₋₃alkyl, or C₃₋₆ cycloalkyl;
each R^{d} is independently halo, —CN, —O—R^{e}, C₁₋₆ alkyl, C₆₋₁₂ aryl or C₁₋₆ haloalkyl; R^{e} is C₁₋₆ alkyl, C₃₋₁₂ cycloalkyl, C₆₋₁₂ aryl, 5-12 membered heteroaryl, each being optionally substituted with C₁₋₆ alkyl or halo; R^{j} is C₁₋₃alkyl or halo; and
L_{c} is

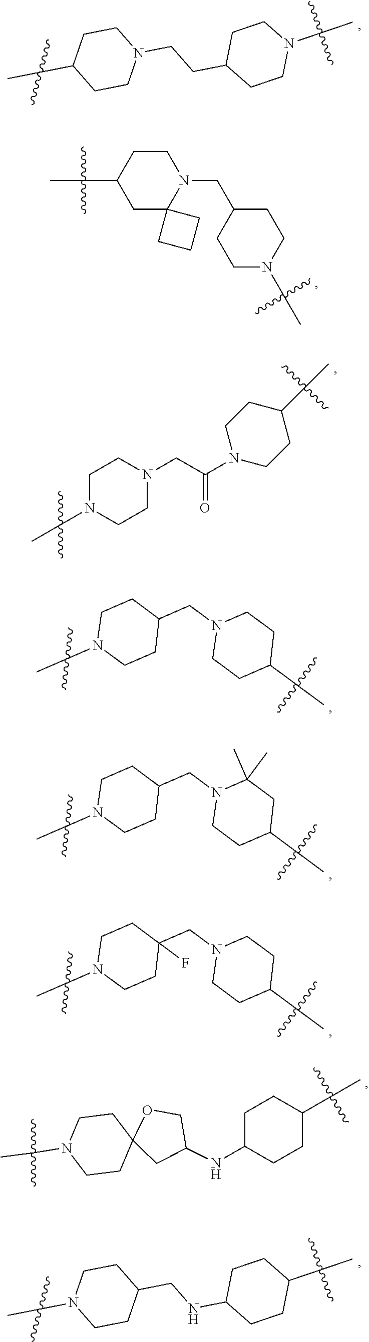

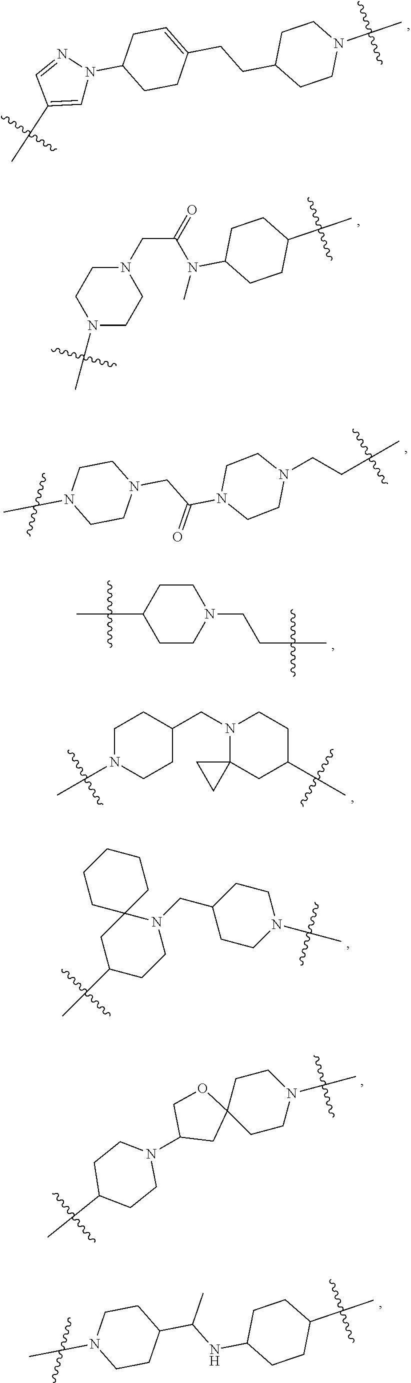

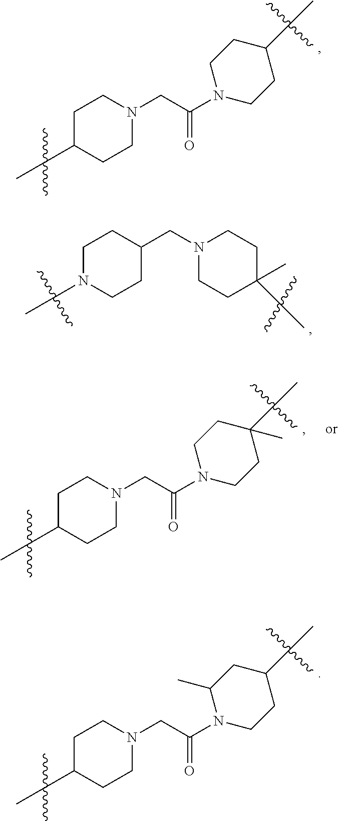 [SMILES: *C1CCN(CC(=O)N2CCC(*)CC2)CC1 ; *N1CCC(CN2CCC(*)(C)CC2)CC1 ; *C1CCN(CC(=O)N2CCC(*)(C)CC2)CC1 ; *C1CCN(CC(=O)N2CCC(*)CC2C)CC1]

In certain embodiments, R¹ and R² are hydrogen, W is CH, Y is direct bond, R³ is phenyl, and the compound has a structure represented by Formula (IA4):

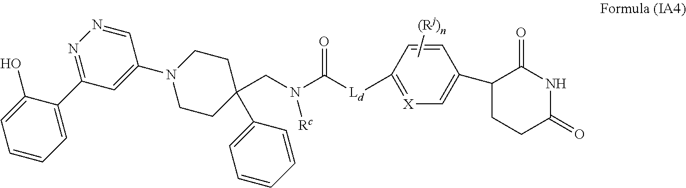

wherein,
n is 0, 1, or 2;
X is N or CH;
R^{e} is hydrogen or C₁₋₃alkyl optionally substituted with C₁₋₃ alkoxy;
R^{j} is C₁₋₃alkyl or halo; and
L_{d} is

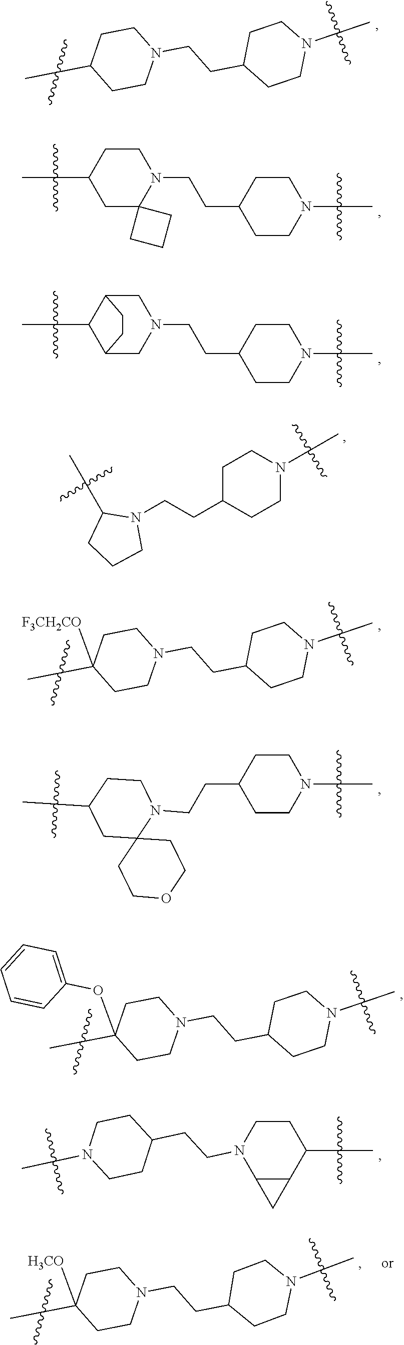

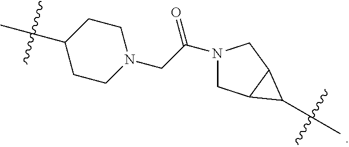 [SMILES: *C1CCN(CC(=O)N2CC3C(*)C3C2)CC1]

In certain embodiments, R¹ and R² are hydrogen, W is CH, Y is direct bond, R³ is phenyl, and the compound has a structure represented by Formula (IA5)

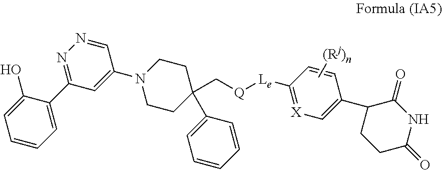

wherein,
n is 0, 1, or 2;
X is N or CH;
Q is direct bond, —N(R^{c})— or —NHS(O)₂—,
R^{c} is hydrogen or C₁₋₃alkyl;
R^{j} is C₁₋₃alkyl or halo; and
Lₑ is

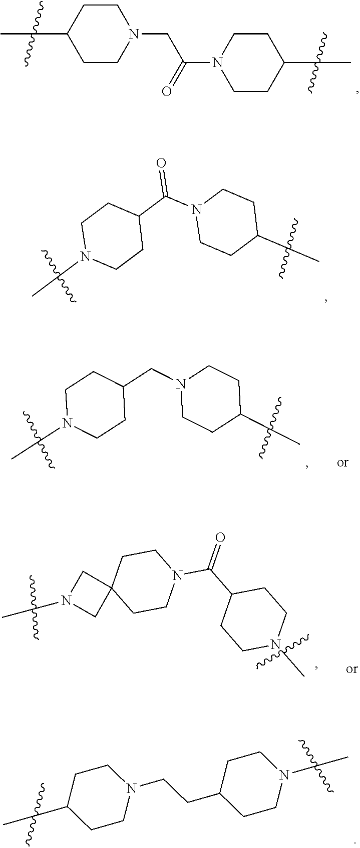 [SMILES: *C1CCN(CC(=O)N2CCC(*)CC2)CC1 ; *C1CCN(C(=O)C2CCN(*)CC2)CC1 ; *C1CCN(CC2CCN(*)CC2)CC1 ; *N1CCC(C(=O)N2CCC3(CC2)CN(*)C3)CC1 ; *C1CCN(CCC2CCN(*)CC2)CC1]

In certain embodiments, R¹ and R² are hydrogen, W is CH, Y is direct bond, and the compound has a structure represented by Formula (IA6):

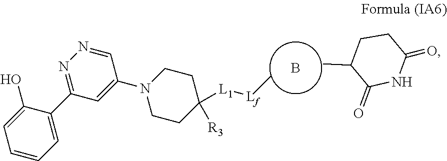

B is

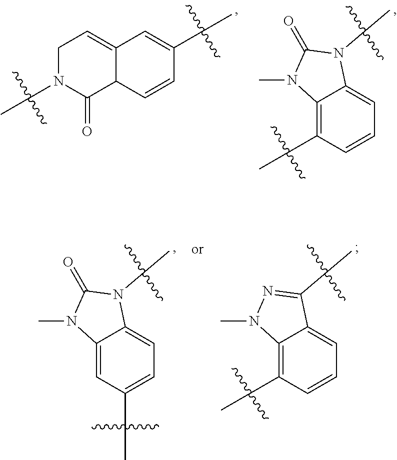 [SMILES: *C1=CC2=CCN(*)C(=O)C2C=C1 ; *c1cccc2c1n(C)c(=O)n2* ; *c1ccc2c(c1)n(C)c(=O)n2* ; *c1nn(C)c2c(*)cccc12]

R³ is C₆₋₁₂ aryl, C₃₋₁₂ cycloalkyl, 5-12 membered heteroaryl, each being optionally substituted with 1 to 3 R^{d}, or —O—R^{e};
R^{c} is hydrogen or C₁₋₆ alkyl;
each R^{d} is independently halo, —CN, —O—R^{c}, C₁₋₆ alkyl, C₆₋₁₂ aryl or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl or halo;
L₁ is —C(O)—, —C(O)—N(R^{e})—(CH₂)—, or —C(O)—N(R^{e})—; and
L_{f} is

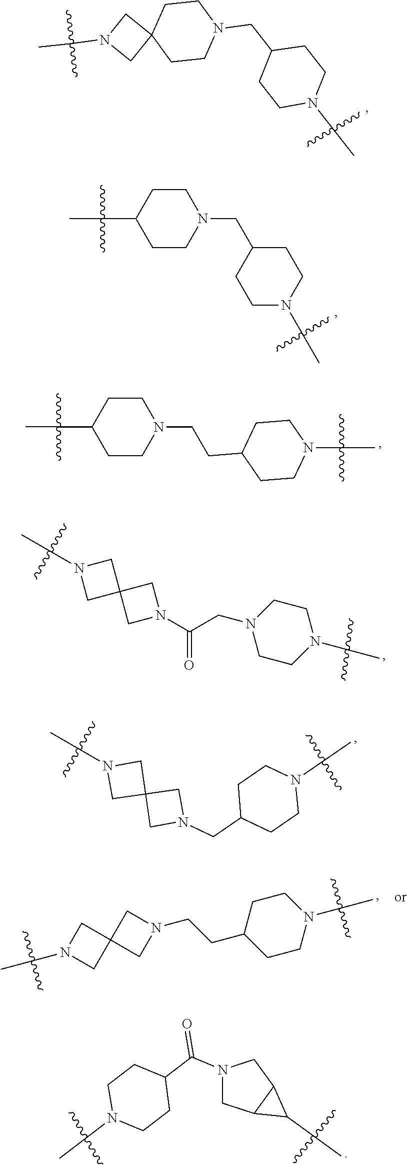

### SMARCA2 Binders

Each bifunctional compound of Formulae (I) and (IA) comprises a selective SMARCA1 binder moiety, depicted as Formula (A):

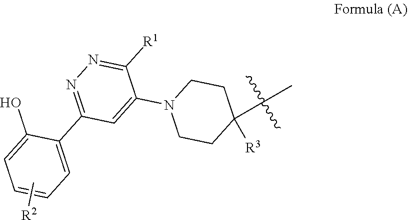

wherein,

In more specific embodiments, R¹ and R² are each hydrogen.

In other more specific embodiments, R¹ is C₁₋₃ alkyl optionally substituted with 1 to 3 halo or hydroxy. In more specific embodiments, R¹ is —CF₂H or —C(CH₃)₂OH, and R² is hydrogen.

In various embodiments, R³ is phenyl, pyridinyl, pyrazolyl, cyclopropyl, or C₁₋₃ alkyl, each being optionally substituted with 1 to 3 R^{d}, wherein R^{d} is C₁₋₃ alkyl, halo, or phenyl.

In more specific embodiments, R³ is phenyl, benzyl, 2-pyridinyl, 3-pyridinyl, —CN, 1-methyl-1H-pyrazol-3yl, 3-methylphenyl, 2-methylphenyl, 3-chlorophenyl, or 3,4-dichlorophenyl.

In various embodiments, R³ is phenyl optionally substituted with one or more C₁₋₆ alkyl, C₁₋₆ alkoxy, or halo; benzyl optionally substituted with one or more C₁₋₆ alkyl or halo; pyridinyl optionally substituted with one or more C₁₋₆ alkyl; CN; C₃₋₆ cyclopropyl; pyrazolyl optionally substituted with one or more C₁₋₆ alkyl; tetrahydropyranyl; 1,2-oxazolyl optionally substituted with one or more C₁₋₆ alkyl; pyrazolo[1,5-a]pyridinyl; or —O—R^{e}, wherein R^{e} is phenyl optionally substituted with C₁₋₆ alkyl or halo, C₃₋₆ cycloalkyl, C₁₋₆ alkyl, or pyrazolyl optionally substituted with C₁₋₆ alkyl.

In even more specific embodiments, R³ is phenyl, benzyl, 2-pyridinyl, 3-pyridinyl, 4-pyridinyl, CN, cyclopropyl, 1-methyl-1H-pyrazol-3yl, 3-methylphenyl, 2-methylphenyl, 3-chlorophenyl, 3,4-dichlorophenyl, 2-methoxyphenyl, 3-methoxyphenyl, 2-fluorophenyl, 4-fluorophenyl, tetrahydropyran-4yl, 3-methyl-1H-pyrazol-1yl, 4-methyl-1H-pyrazol-1yl, 5-methyl-1H-pyrazol-1yl, 1-methyl-1H-pyrazol-3yl, 1-methyl-1H-pyrazol-4yl, 1-methyl-1H-pyrazol-5yl, pyrazolo[1,5-a]pyridin-2-yl, 5-methyl-1,2-oxazol-3-yl, 5-isopropyl-1,2-oxazol-3-yl, 3-methyl-1,2-oxazol-5-yl, or —O—R^{e}, wherein R^{e} is phenyl, 2-chlorophenyl, 2-methylphenyl, cyclopropyl, cyclohexyl, methyl, or 1-methyl-1H-pyrazol-3yl.

In a specific embodiment, R¹ and R² are each hydrogen, R³ is phenyl, and the SMARCA2 binder moiety has the structure of Formula (A1):

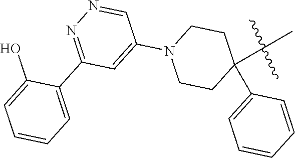 [SMILES: *C1(c2ccccc2)CCN(c2cnnc(-c3ccccc3O)c2)CC1]

In another specific embodiment, R¹ and R² are each hydrogen, R³ is —O—R^{e}, as defined herein.

### Ligase Harness Moieties (LHM)

The LHM moiety of the bifunctional compound of Formula (I) targets CRBN of E3 ligases. Once harnessed by the bifunctional compounds, the E3 ligases are capable of inducing ubiquitination and subsequent proteasomal degradation of SMARCA2.

The LHM moiety of Formula (I) typically comprises a glutarimide or dihydrouracil moiety coupled to a ring structure, as represented by Formula (B):

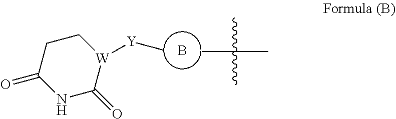

wherein,
W is —C(R^{g})— or —N—;
Y is direct bond, C₁₋₄ alkylene chain, —C(O)—, —C(O)O—, —O—, —N(R^{g})—, —S—
—C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R^{j};
each R^{j} is independently halo, —CN, oxo, —O—R^{e}, C₁₋₆ alkyl, or C₁₋₆ haloalkyl; and
R^{g} is hydrogen or C₁₋₆ alkyl.

In more specific embodiments, W is —CH—, Y is direct bond; and B ring is

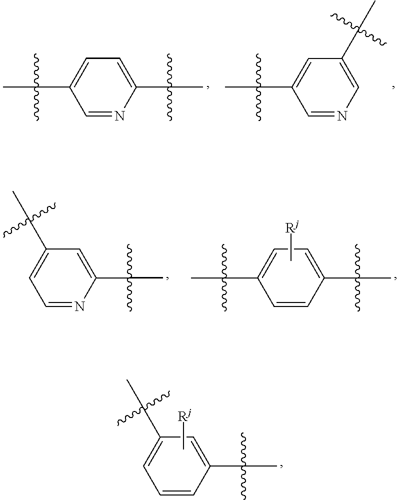

wherein R^{j} is H, halo, or C₁₋₃alkyl.

In yet other embodiments W is —CH—, Y is —NHC(O)— or —NH—, and B ring is

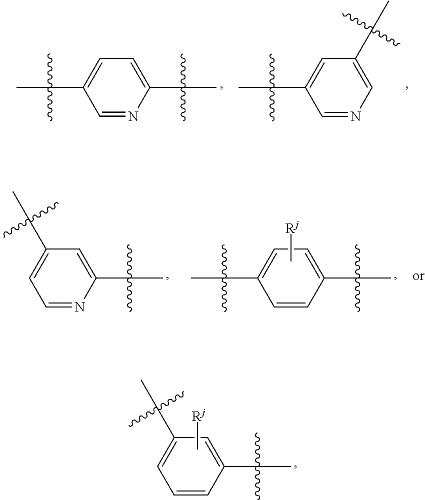

wherein R^{j} is H, halo, or C₁₋₃alkyl.

In other embodiments, W is —N—, Y is direct bond; and B ring is

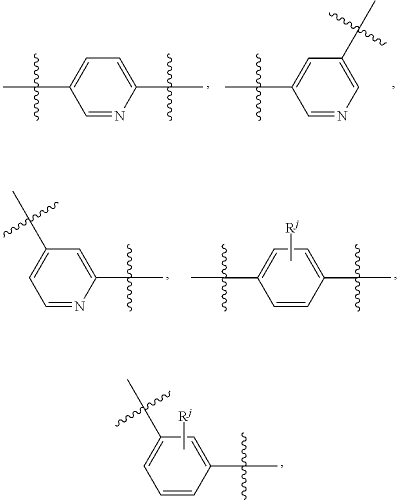

wherein R^{j} is H or C₁₋₃alkyl.

In more specific embodiments, the LHM moiety is:

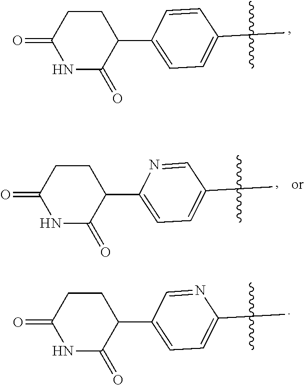

In other more specific embodiments, the LHM moiety is:

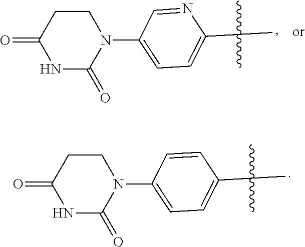 [SMILES: *c1ccc(N2CCC(=O)NC2=O)cn1 ; *c1ccc(N2CCC(=O)NC2=O)cc1]

In other more specific embodiments, the LHM moiety is:

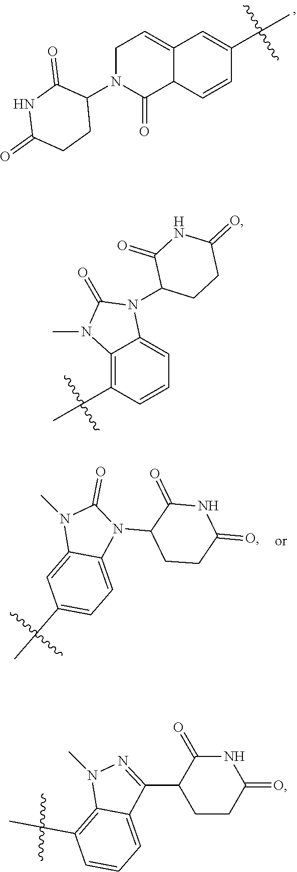 [SMILES: *C1=CC2=CCN(C3CCC(=O)NC3=O)C(=O)C2C=C1 ; *c1cccc2c1n(C)c(=O)n2C1CCC(=O)NC1=O ; *c1ccc2c(c1)n(C)c(=O)n2C1CCC(=O)NC1=O ; *c1cccc2c(C3CCC(=O)NC3=O)nn(C)c12]

### Linker

Each bifunctional compound of Formula (I) comprises a Linker (L), which is a bivalent moiety that couples the SMARCA2 Binder moiety to the LHM. The structure (e.g., length or rigidity) of the linker moiety may impact the efficiency or selectivity of the degradation process.

The Linker comprises a continuous sequence of covalent bonds between the respective attachment points to the SMARCA2 Binder moiety and the LHM, inclusive of the bond indicated by a wavy line of Formulae (A) and (B) and their respective substructures.

Typically, the linker moiety comprises multiple bivalent segments (i.e., -L₁-L₂-L₃-L₄-L₅-L₆-L₇-L₈-), which collectively contribute to the overall length and rigidity of the Linker, in addition to providing the respective attachment points to the SMARCA2 Binder moiety and the LHM. In various embodiments, the linker segments L₁, L₂, L₃, L₄, L₅, L₆, L₇, L₈ are each independently:
i) C₃₋₁₂ cycloalkyl optionally substituted with 1-3 R^{b};
ii) C₆₋₁₂ aryl optionally substituted with 1-3 R^{b};
iii) 4-12 membered heterocyclyl optionally substituted with 1-3 R^{b};
iv) 5-12 membered heteroaryl optionally substituted with 1-3 R^{b};
v) direct bond;
vi) C₁₋₁₂ alkylene chain optionally substituted with 1-3 R^{d}; or
vii) —(CH₂)ₘ—C(O)—, —(CH₂)ₘ—C(O)O—, —(CH₂)ₘ—O—, —(CH₂)ₘ—N(R^{c})—,
—(CH₂)ₘ—S—, —(CH₂)ₘ—C(S)—, —(CH₂)ₘ—C(S)—O—, —(CH₂)ₘ—S(O)₂—, —(CH₂)ₘ—S(O)═N—
—(CH₂)ₘ—S(O)₂NH—, —(CH₂)ₘ—C(O)—N(R^{c})—, —C(O)—N(R^{c})—(CH₂)ₘ—, —CH₂)ₘ—O—C(O)—N(R^{c})—,
—(CH₂)ₘ—O—C(O)—O—, or —NH—(CH₂)ₘ—C(O)—, wherein m is 0, 1, 2, 3, 4, 5 or 6;
wherein R^{d}, R^{c} and R^{d} are as defined herein.

It is to be understood that, unless otherwise specified and provided that the valence is satisfied, the bivalent linker segments described herein (e.g., L¹ or L²) are not limited to the direction in which they are expressed. For instance, for a given linker segment, e.g., —C(O)—NH—, the manner in which it is connected to the remainder of the molecule may be either direction: i.e., —C(O)—NH— or —NH—C(O)—, provided that the connection does not violate valence rules.

Unless other specified, it is further to be understood that the first linker segment L¹ is directly coupled to the SMARCA2 Binder moiety, whereas the last linker segment L⁸ is directly coupled to the LHM.

One or more linker segments may be direct bonds. To illustrate, in a sequence of linker segments represented by -L₂-L₃-L₄-, when L₃ is a direct bond, it is effectively absent because L₂ and L₄ are coupled directly to each other.

In another embodiment, the Linker has one or more rings, which tend to increase the linker rigidity. A combination of chain bonds and ring(s) may be used to tune the relative orientations of the bifunctional groups or the distance therebetween.

In various specific embodiments, the linker (L) of compounds of Formulae (I), (II), (11a), (IIb), (IIc) and (IId) is presented by -L₁-L₂-L₃-L₄-L₅-L₆-L₇-L₈-, each L₁, L₂, L₃, L₄, L₅, L₆, L₇, or L₈, being independently:
i)

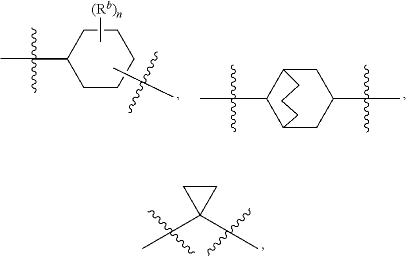

ii)

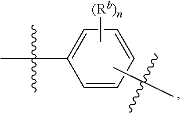

iii)

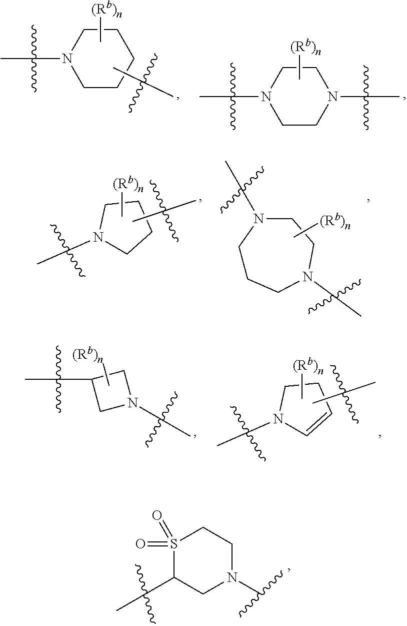

iv)

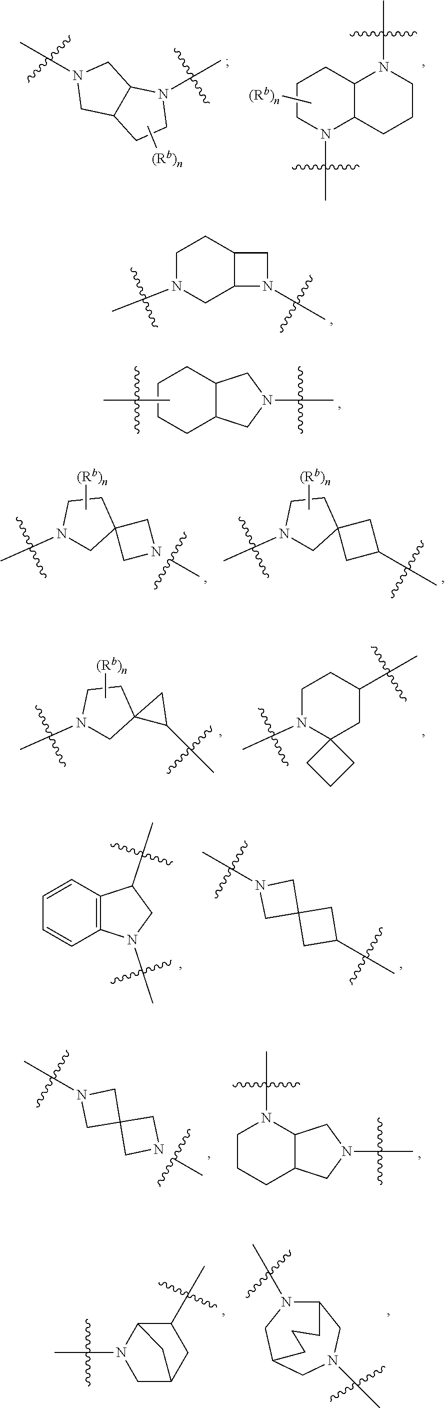

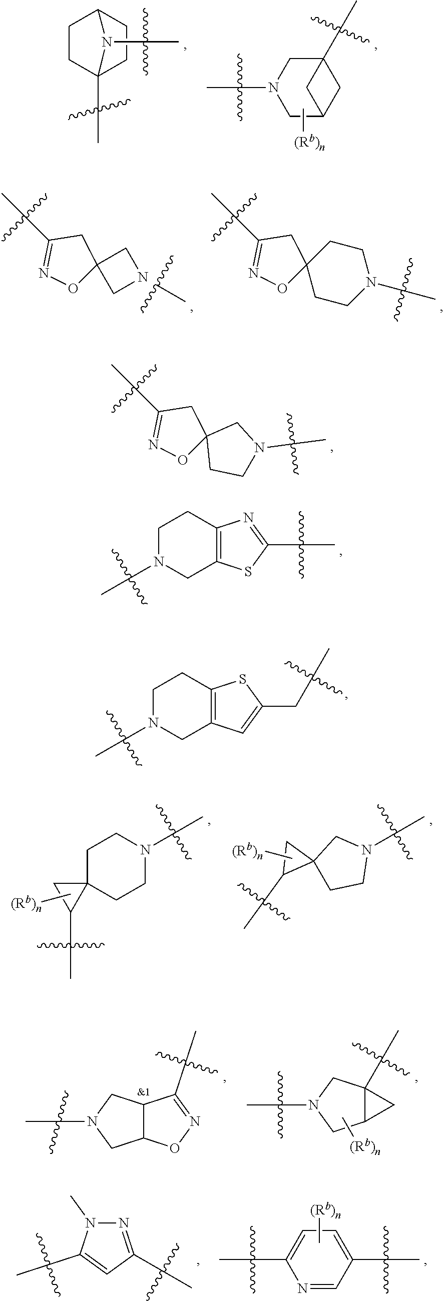

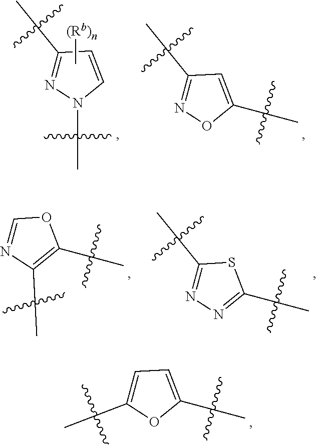

iv)

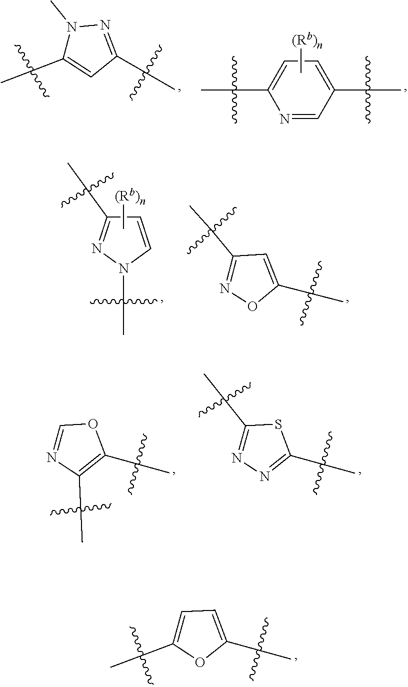

v) direct bond;
vi) C₁₋₆ alkylene chain; or
vii) —C(O)—, —O—, —C(O)—N(R′)—, —(CH₂)ₘ—C(O)—, or —NH—(CH₂)ₘ—C(O)—, wherein m is 0, 1, 2 or 3;
wherein,
n is 0, 1, or 2;
R^{b} is halo, —CN, C₁₋₃ alkyl, or C₁₋₃ haloalkyl; and
R^{c} is hydrogen or C₁₋₃ alkyl.

In more specific embodiments, one or more of the linker segments form a diamine linker moiety having one of the following structures:

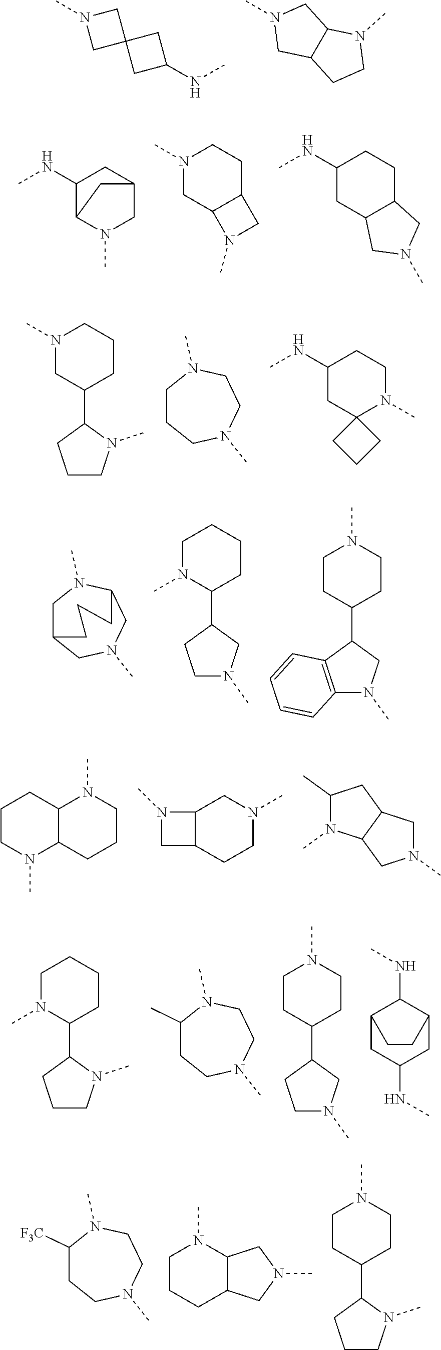

In other specific embodiments, the linker (L′) of compounds of Formula (III) and its substructures Formulae (IIIa), (IIIb), (IIIc) and (IIId) may have one of the following structures:

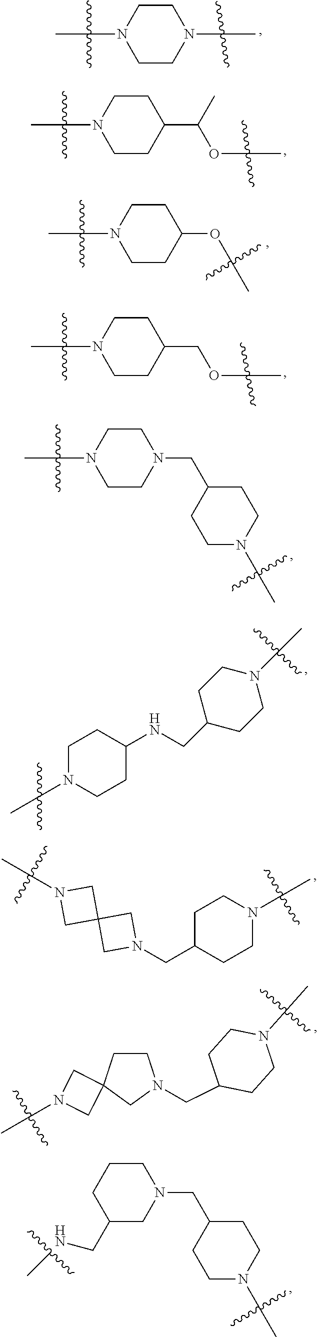

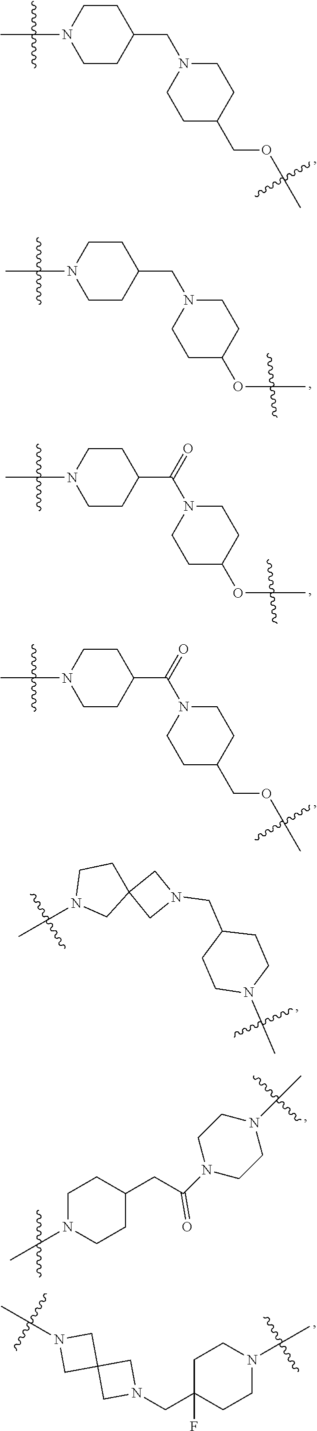

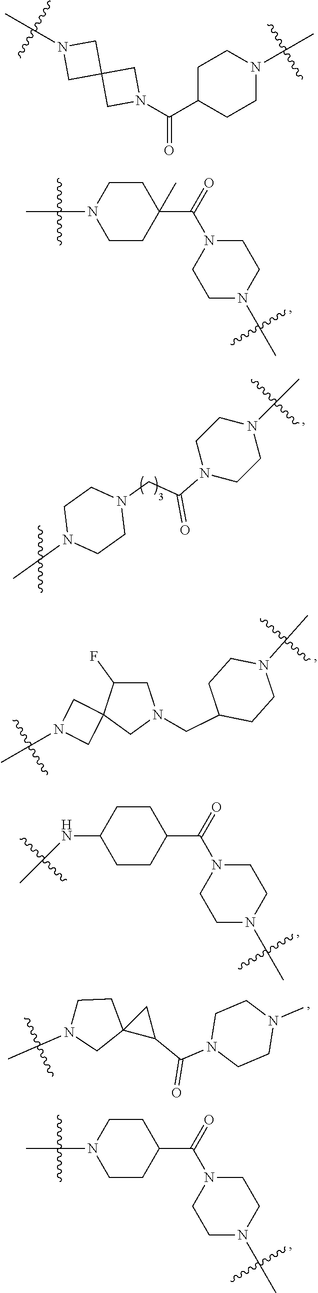

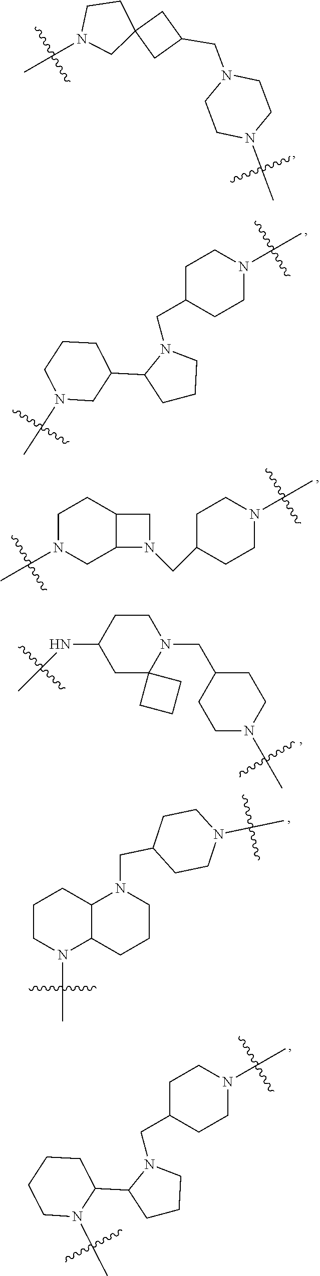 [SMILES: *N1CCN(CC2CC3(CCN(*)C3)C2)CC1 ; *N1CCC(CN2CCCC2C2CCCN(*)C2)CC1 ; *N1CCC(CN2CC3CCN(*)CC32)CC1 ; *NC1CCN(CC2CCN(*)CC2)C2(CCC2)C1 ; *N1CCC(CN2CCCC3C2CCCN3*)CC1 ; *N1CCC(CN2CCCC2C2CCCCN2*)CC1]

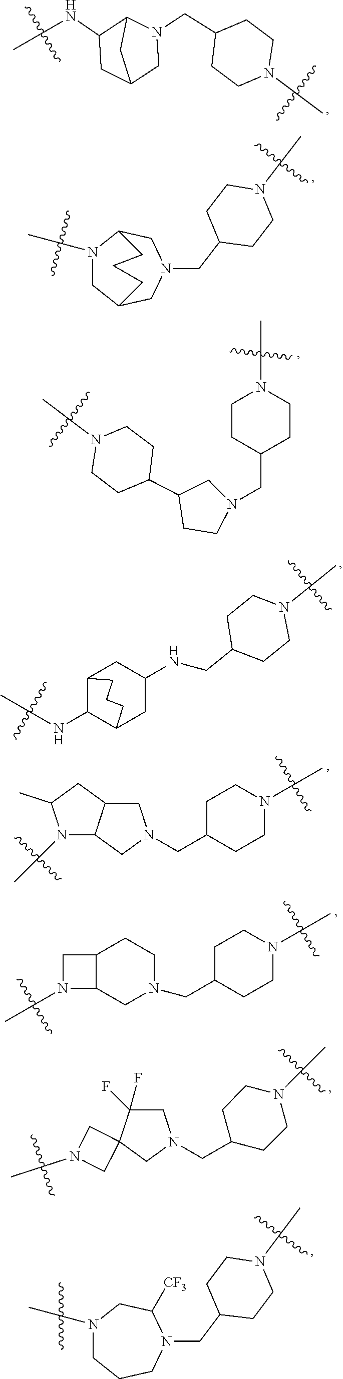

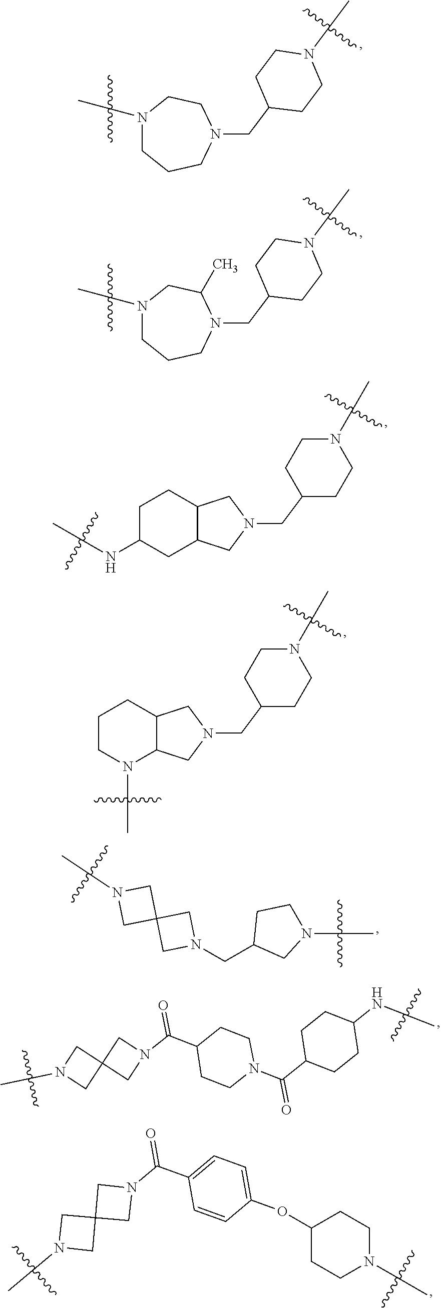

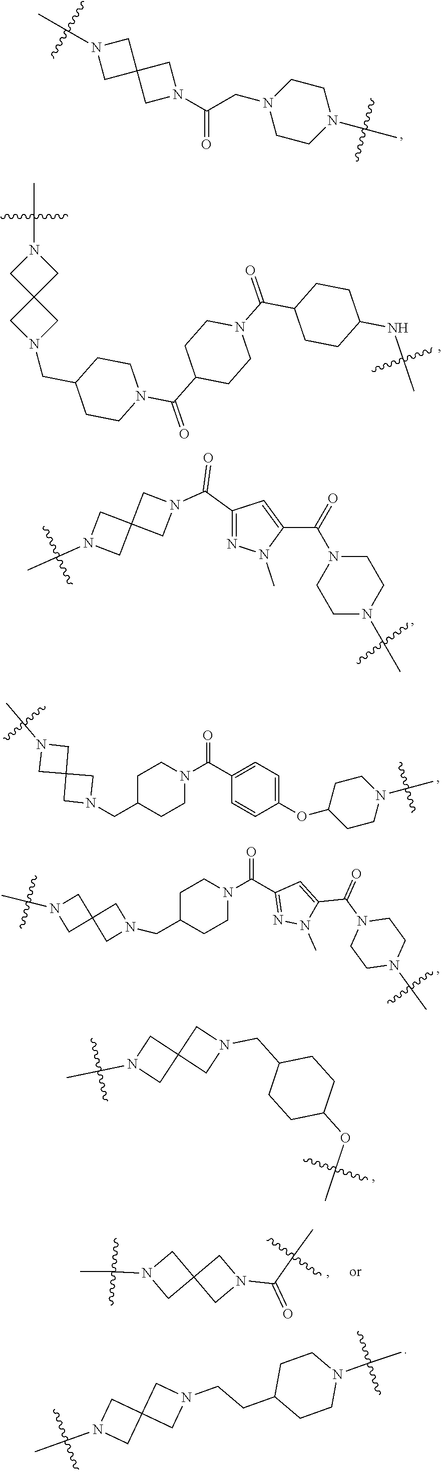

In yet other specific embodiments, the linker (L″) of compounds of Formulae (IV), (IVa) and (IVb) and their respective substructures, may have one of the following structures:

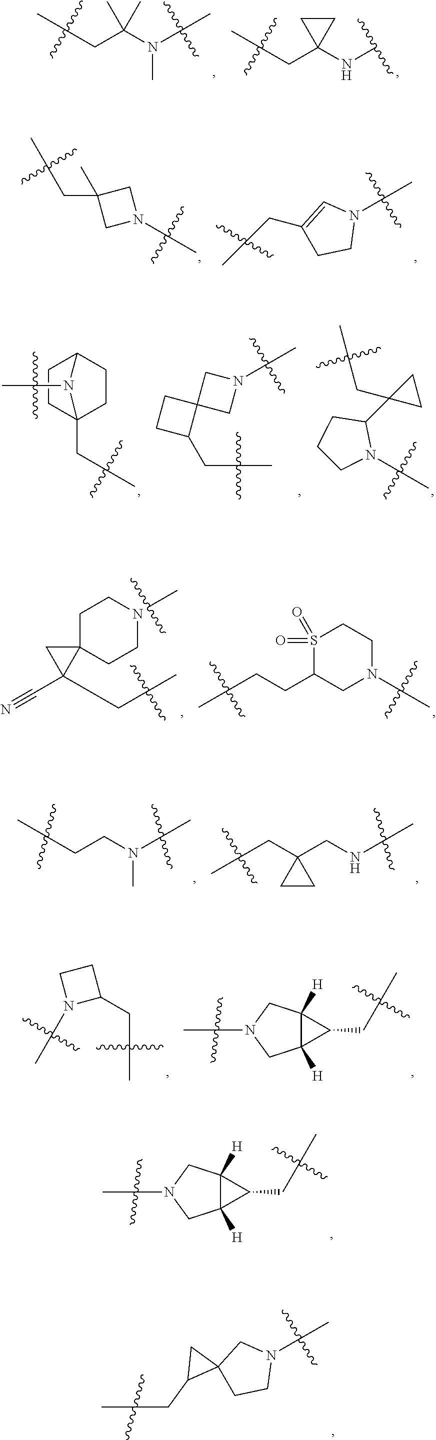

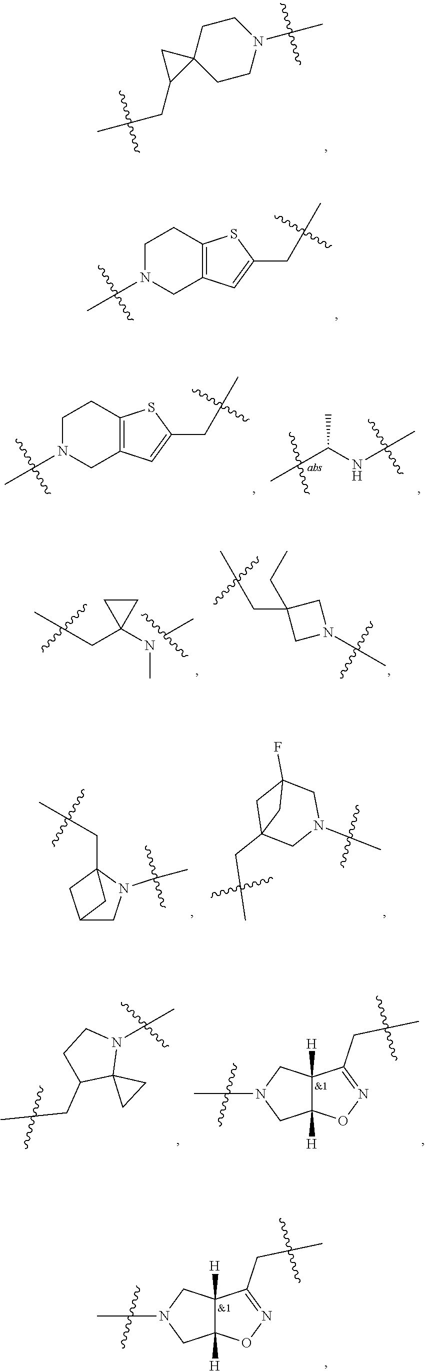

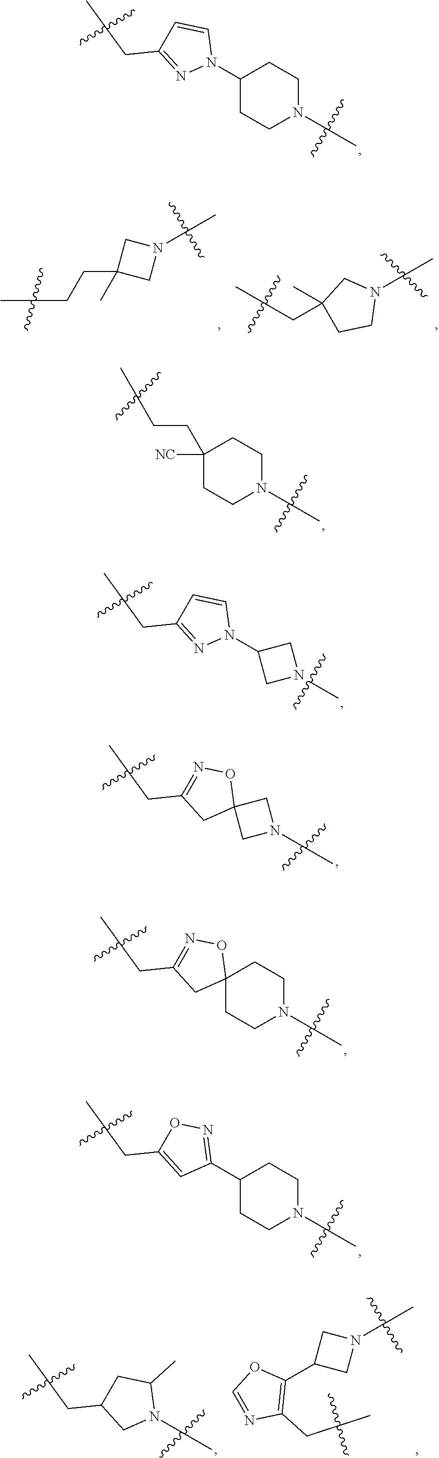

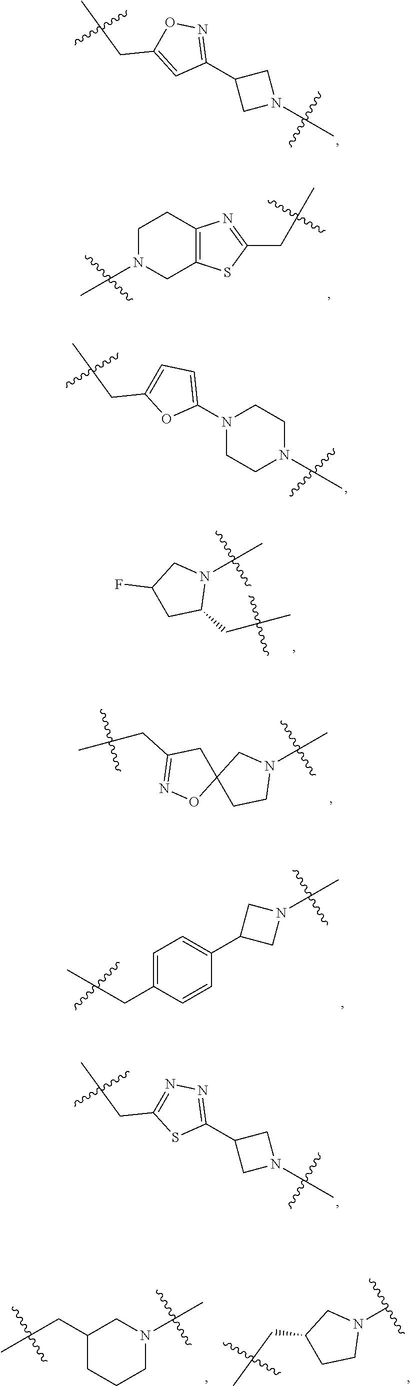

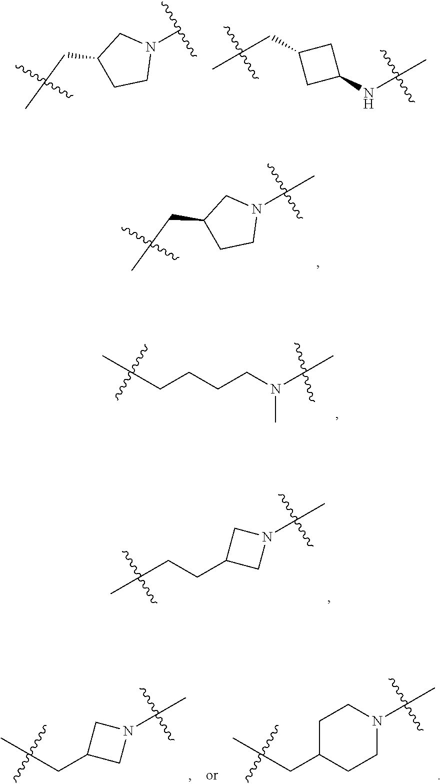

### Definitions

The following description sets forth exemplary methods, parameters and the like. It should be recognized, however, that such description is not intended as a limitation on the scope of the present disclosure but is instead provided as a description of exemplary embodiments.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain containing no unsaturation. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

“Alkylene” or “alkylene chain” refers to a unbranched or branched divalent hydrocarbon chain, linking the rest of the molecule to a radical group, containing no unsaturation and having from 1 to 20 carbon atoms, or more typically 1 to 12 carbon atoms (C₁₋₁₂ alkylene), or 1 to 8 carbon atoms (C₁₋₈ alkylene), or 1 to 3 carbon atoms (C₁₋₃ alkylene) e.g., methylene, ethylene, propylene, n-butylene, and the like. The alkylene chain may be attached to the rest of the molecule and to the radical group through one carbon within the chain or through any two carbons within the chain.

“Alkenyl” refers to an alkyl group containing at least one carbon-carbon double bond and having from 2 to 20 carbon atoms (i.e., C₂₋₂₀ alkenyl), or more typically 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkenylene” and “alkenylene chain” refer to a unbranched or branched divalent hydrocarbon chain linking the rest of the molecule to a radical group, containing at least one double bond and having from 2 to 20 carbon atoms, or more typically 2 to 12 carbon atoms, or 2 to 8 carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain.

“Alkynyl” refers to an alkyl group containing at least one carbon-carbon triple bond and having from 2 to 20 carbon atoms (i.e., C₂₋₂₀ alkynyl), or more typically 2 to 12 carbon atoms (i.e., C₂₋₁₂ alkynyl), or more typically 2 to 8 carbon atoms (i.e., C₂₋₈ alkynyl), 2 to 6 carbon atoms (i.e., C₂₋₆alkynyl), or 2 to 4 carbon atoms (i.e., C₂₋₄ alkynyl). The term “alkynyl” also includes those groups having one triple bond and one double bond.

“Alkynylene” and “alkynylene chain” refer to a unbranched or branched divalent hydrocarbon chain linking the rest of the molecule to a radical group, containing at least one triple bond and having from 2 to 20 carbon atoms, or more typically 2 to 12 carbon atoms, or 2 to 8 carbon atoms. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy.

“Haloalkoxy” refers to an alkoxy group as defined above, wherein one or more hydrogen atoms are replaced by a halogen.

“Alkylthio” refers to the group “alkyl-S—”.

“Amino” refers to the group —NR^{y}R^{y} wherein each R^{y} is independently selected from the group consisting of hydrogen, alkyl, alkenyl, alkynyl, aryl, heterocyclyl, cycloalkyl or heteroaryl, each of which is optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 15 carbon ring atoms (i.e., C₆₋₁₅ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cyano” refers to the group —CN.

“Keto” or “oxo” refers to a group ═O.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently selected from the group consisting of hydrogen, alkyl, aryl, haloalkyl, or heteroaryl; each of which may be optionally substituted.

“Carboxyl” or “carboxylic acid” refers to —C(O)OH.

“Ester” refers to both —OC(O)R and —C(O)OR, wherein R is a substituent; each of which may be optionally substituted, as defined herein.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond). As used herein, cycloalkyl has from 3 to 15 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, cyclohexyl, and bicyclo[2.2.2]octan-1-yl. Cycloalkyl may be attached to the remainder of a molecule by a single ring atom (e.g., as a substituent) or by two ring atoms (e.g., as a linker).

“Ethylene glycol unit” refers to a bivalent monomer having the structure of —CH₂CH₂O—, which may be repeated and extended into a longer chain. A linker segment may have up to 12 ethylene glycol units, or more typically up to 6 ethylene glycol units.

“Propylene glycol unit” refers to a bivalent monomer having the structure of —CH(CH₃)—CH₂O—, which may be repeated and extended into a longer chain. A linker segment may have up to 12 propylene glycol units, or more typically up to 6 propylene glycol units.

“Halogen” or “halo” includes fluoro, chloro, bromo, and iodo.

“Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Heteroalkyl” refers to an alkyl group in which one or more of the carbon atoms (and any associated hydrogen atoms) are each independently replaced with the same or different heteroatoms such as N, O, S, and the likes. The term “heteroalkyl” includes unbranched or branched saturated chain having carbon and heteroatoms. By way of example, 1, 2 or 3 carbon atoms may be independently replaced with the same or different heteroatoms. Heteroatomic groups include, but are not limited to, —N(R)—, —O—, —S—, —S(O)—, —S(O)₂—, and the like, where R is H, alkyl, aryl, cycloalkyl, heteroalkyl, heteroaryl or heterocyclyl, each of which may be optionally substituted. Examples of heteroalkyl groups include —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl include 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to a 5-15 membered, or more typically, 5-12 membered aromatic group having a single ring, multiple rings, or multiple fused rings, with 1-3 ring heteroatoms independently selected from nitrogen, oxygen, and sulfur. As used herein, heteroaryl includes 3 to 12 ring carbon atoms (i.e., C₃₋₁₂ heteroaryl), or 3 to 8 carbon ring atoms (i.e., C₃₋₈ heteroaryl); and 1 to 5 heteroatoms, 1 to 4 heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, and pyrazolyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl (which has no heteroatom) or heterocyclyl (which has at least one non-aromatic ring). Heteroaryl may be attached to the remainder of a molecule by a single ring atom (e.g., as a substituent) or by two ring atoms (e.g., as a linker).

“Heterocyclyl” refers to a 3-15 membered, or more typically, 5-12 membered, saturated or unsaturated cyclic alkyl group, with 1-3 ring heteroatoms independently selected from nitrogen, oxygen and sulfur. The term “heterocyclyl” includes heterocycloalkenyl groups (i.e., the heterocyclyl group having at least one double bond), bicyclic heterocyclyl groups, bridged-heterocyclyl groups, fused-heterocyclyl groups, and spiro-heterocyclyl groups. A heterocyclyl may be a single ring or multiple rings wherein the multiple rings may be fused, bridged, or spiro. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 3 to 15 ring atoms (e.g., 3-15 membered heterocyclyl, 3-12 membered heterocyclyl, 4 to 10 membered heterocyclyl, 4-8 membered heterocyclyl or 4-6 membered heterocyclyl; having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. A heterocyclyl may contain one or more oxo and/or thioxo groups. Examples of heterocyclyl groups include pyrrolidinyl, piperidinyl, piperazinyl, oxetanyl, dioxolanyl, azetidinyl, azetidinyl, morpholinyl, thiomorpholinyl, 4-7 membered sultam, 4-7 membered cyclic carbamate, 4-7 membered cyclic carbonate, 4-7 membered cyclic sulfide and morpholinyl. As used herein, heterocyclyl may include a bridged structure (i.e., “bridged heterocyclyl), in which a four- to ten-membered cyclic moiety connected at two non-adjacent atoms of the heterocyclyl with one or more (e.g., 1 or 2) four- to ten-membered cyclic moiety having at least one heteroatom where each heteroatom is independently selected from nitrogen, oxygen, and sulfur. As used herein, bridged-heterocyclyl includes bicyclic and tricyclic ring systems. Also used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Examples of the spiro-heterocyclyl rings include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 1-oxo-1,2,3,4-tetrahydroisoquinolinyl, 1-oxo-1,2-dihydroisoquinolinyl, 4,5,6,7-tetrahydrothieno[2,3-c]pyridinyl, indolinyl, and isoindolinyl, where the heterocyclyl can be bound via either ring of the fused system. As used herein, a bicyclic heterocyclyl group is a heterocyclyl group attached at two points to another cyclic group, wherein the other cyclic group may itself be a heterocyclic group, or a carbocyclic group. Heteroaryl may be attached to the remainder of a molecule by a single ring atom (e.g., as a substituent) or by two ring atoms (e.g., as a linker).

“Fused” refers to a ring which is joint to an adjacent ring and share two adjacent ring atoms that form a covalent bond.

“Bridged” refers to a ring fusion wherein non-adjacent atoms on a ring are joined by a divalent substituent, such as alkylenyl group, an alkylenyl group containing one or two heteroatoms, or a single heteroatom. Quinuclidinyl and admantanyl are examples of bridged ring systems.

“Spiro” refers to a ring substituent which is joined by two bonds at the same carbon atom. Examples of spiro groups include 1,1-diethylcyclopentane, dimethyl-dioxolane, and 4-benzyl-4-methylpiperidine, wherein the cyclopentane and piperidine, respectively, are the spiro substituents.

“Hydroxy” or “hydroxyl” refers to the group —OH. “Hydroxyalkyl” refers to an unbranched or branched alkyl group as defined above, wherein one or more hydrogen atoms are replaced by a hydroxyl.

“Nitro” refers to the group —NO₂.

“Imino” refers to a group that contains a C═N double bond, such as C═N—R^{y}, or ═N—C(O)R^{y}, wherein R^{y} is selected from the group consisting of hydrogen, alkyl, aryl, cyano, haloalkyl, or heteroaryl; each of which may be optionally substituted. Imino may be a linker segment by attaching to the remainder molecule at the carbon and nitrogen respectively.

“Sulfonyl” refers to the group —S(O)₂R, where R is a substituent, or a defined group.

“Alkylsulfonyl” refers to the group —S(O)₂R, where R is a substituent, or a defined group.

“Alkylsulfinyl” refers to the group —S(O)R, where R is a substituent, or a defined group.

“Thiocyanate” —SCN.

“Thiol” refers to the group —SR, where R is a substituent, or a defined group.

“Thioxo” or “thione” refer to the group (═S) or (S).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl, the last mentioned group contains the atom by which the moiety is attached to the rest of the molecule.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen. “Optionally substituted” may be zero to the maximum number of possible substitutions, and each occurrence is independent. When the term “substituted” is used, then that substitution is required to be made at a substitutable hydrogen atom of the indicated substituent. An optional substitution may be the same or different from a (required) substitution.

When a moiety is “optionally substituted,” and reference is made to a general term, such as any “alkyl,” “alkenyl,” “alkynyl,” “haloalkyl,” “cycloalkyl,” “aryl” or “heteroaryl,” then the general term can refer to any antecedent specifically recited term, such as (C₁₋₃ alkyl), (C₄₋₆ alkyl), —O(C₁₋₄ alkyl), (C₃₋₁₀ cycloalkyl), O—(C₃₋₁₀ cycloalkyl) and the like. For example, “any aryl” includes both “aryl” and “—O(aryl) as well as examples of aryl, such as phenyl or naphthyl and the like. Also, the term “any heterocyclyl” includes both the terms “heterocyclyl” and O-(heterocyclyl),” as well as examples of heterocyclyls, such as oxetanyl, tetrahydropyranyl, morpholino, piperidinyl and the like. In the same manner, the term “any heteroaryl” includes the terms “heteroaryl” and “O-(heteroryl),” as well as specific heteroaryls, such as pyridine and the like.

Some compounds of Formula (I) may exist as a “stereoisomer” or a mixture of stereoisomers. Stereoisomer refers to a compound made up of the same atoms bonded by the same bonds but having different three-dimensional structures, which are not interchangeable. The compounds of the disclosure, or their pharmaceutically acceptable salts may contain one or more asymmetric centers and may thus give rise to enantiomers (two stereoisomers whose molecules are non-superimposable mirror images of one another), diastereomers, and other stereoisomeric forms that may be defined, in terms of absolute stereochemistry, as (R)- or (S)-. The present disclosure is meant to include all such possible isomers, as well as their racemic mixture (i.e., equal amounts of (R) and (S) enantiomers) and optically pure forms. Optically active (+) and (−), (R)- and (S)-isomers may be prepared using chiral synthons or chiral reagents, or resolved using conventional techniques, such as HPLC or SFC using a chiral column.

“& 1” means that a compound including the “& 1” notation at a particular chemical element or atom (e.g., carbon) within the compound was prepared as a mixture of two stereoisomers at the noted chemical element or atom (e.g., a diastereomeric mixture having a de or % de as described above). “&2,” if presents, denotes a second set of isomers.

The disclosure also includes “deuterated analogues” of compounds of Formula (I) in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula (I) when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula (I).

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts, hydrates, or solvates of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical uses.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, haloalkoxy, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted One skilled in the art will recognize that substituents and other moieties of the compounds of the generic formula herein should be selected in order to provide a compound which is sufficiently stable to provide a pharmaceutically useful compound which can be formulated into an acceptably stable pharmaceutical composition. Compounds which have such stability are contemplated as falling within the scope of the present invention. It should be understood by one skilled in the art that any combination of the definitions and substituents described above should not result in an inoperable species or compound.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

### Targeted SMARCA2 Degradation

The compounds of the present disclosure are demonstrated by cell-based profiling to selectively degrade SMARCA2 while sparing SMARCA4.

Additional biological activities, including selectivity data of compounds of Formula (I) are summarized in Table 1 herein.

### Pharmaceutical Composition and Use of the Bifunctional Compounds of Formula (I)

The bifunctional compounds of Formula (I) are demonstrated to selectively degrade SMARCA2 and are therefore particularly useful for treating SMARCA4-deficient cancers.

Various embodiments provide pharmaceutical compositions of a compound of Formula (I), or any one of the substructures or specific compounds of Examples 1-58, and a pharmaceutically acceptable carrier.

Further embodiments provide methods for treating SMARCA2-mediated disease or disorder, including, for increasing T-cell activation, for treating cancer, for inhibiting the growth or proliferation of cancer cells, the method comprising administering to a subject in need thereof a therapeutically effective amount of a compound of Formula (I), any one of the substructures or compounds of Examples 1-58.

SMARCA2-mediated diseases may be cancers selected from the group consisting of acoustic neuroma, acute leukemiat, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, liver cancer, lung cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In some embodiments, a compound of Formula (I), or any one of the substructures or a compound of Examples 1-58 may be co-administered with a therapeutically effective amount of one or more additional therapeutic agents, or a pharmaceutically acceptable salt thereof. The additional therapeutic agents include, for example, chemotherapeutic agents disclosed in WO WO2021083949.

### Construction of Compounds of Formula (I)

The synthesis or construction of the compounds of Formula (I) can be carried out in multiple steps, typically involving separately preparing building blocks of the SMARCA2 binder and the LHM moiety, followed by joining the respective building blocks through covalent bond formation. Generally speaking, either or both building blocks may be prepared with one or more linker precursors. A linker precursor comprises one or more linker segments (Lₛ) and has a terminal reactive group for further coupling. The two building blocks can be finally coupled (via formation of a further linker segment) to afford a compound of Formula (I).

The following schemes demonstrate the general approaches of preparing building blocks. Specific examples (Examples 1-58) were synthesized and characterized by their respective physiochemical properties according to the general schemes described herein.

### INTERMEDIATES

### Intermediate 1

### 1-[6-(2-hydroxyphenyl)pyridazin-4-yl]-4-(5-methyl-isoxazol-3-yl)piperidine-4-carboxylic acid

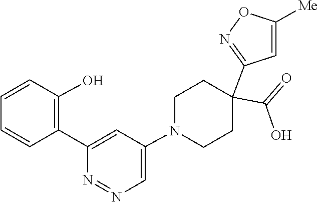 [SMILES: Cc1cc(C2(C(=O)O)CCN(c3cnnc(-c4ccccc4O)c3)CC2)no1]

### Step 1: Synthesis of 1-(tert-butyl) 4-methyl 4-(5-methylisoxazol-3-yl)piperidine-1,4-dicarboxylate

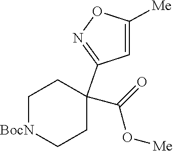 [SMILES: COC(=O)C1(c2cc(C)on2)CCN(C(=O)OC(C)(C)C)CC1]

To a solution of methyl 2-(5-methylisoxazol-3-yl)acetate (28.0 g, 180 mmol, 1.00 eq) in DMF (280 mL) was added NaH (18.0 g, 451 mmol, 60% purity, 2.50 eq) at 0-5° C. under N₂, the mixture was stirred at 0-5° C. for 1 hr. Then, tert-butyl bis(2-chloroethyl)carbamate (48.0 g, 198 mmol, 1.10 eq) was added at 0-5° C., the mixture was heated to 60° C. and stirred at 60° C. for 15 hrs. The mixture was cooled down to 25° C., then poured into saturated NH₄Cl (500 mL) aqueous solution, then extracted with EtOAc (250 mL*2). The combined organic layers were washed with brine (250 mL*2), dried over Na₂SO₄, filtered and concentrated to give the product as a black brown liquid (57.8 g, crude). The crude product was directly used in the next step. LCMS: C₁₆H₂₄N₂O₅ requires 324.4. found: m/z=325.2 [M+H]⁺.

### Step 2: Synthesis of methyl 4-(5-methylisoxazol-3-yl)piperidine-4-carboxylate

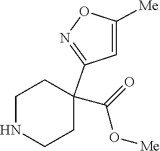 [SMILES: COC(=O)C1(c2cc(C)on2)CCNCC1]

A solution of 1-(tert-butyl) 4-methyl 4-(5-methylisoxazol-3-yl)piperidine-1,4-dicarboxylate (57.8 g, 178 mmol, 1.00 eq) in HCl/EtOAc (2 M, 578 mL, 6.49 eq) was stirred at 20-25° C. for 2 hrs. The reaction mixture was filtered and filter cake was dried under vacuum. The crude product was directly used in the next step. Methyl 4-(5-methylisoxazol-3-yl)piperidine-4-carboxylate (49.7 g, crude, HCl) was obtained as black brown liquid. LCMS: C₁₁H₁₆N₂O₃ requires 224.1. found: m/z=225.1 [M+H]⁺.

### Step 3: Synthesis of methyl 1-(6-chloropyridazin-4-yl)-4-(5-methylisoxazol-3-yl)piperidine-4-carboxylate

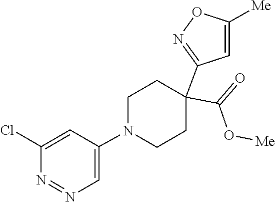 [SMILES: COC(=O)C1(c2cc(C)on2)CCN(c2cnnc(Cl)c2)CC1]

To a solution of methyl 4-(5-methylisoxazol-3-yl)piperidine-4-carboxylate hydrochloride (47.7 g, 183 mmol, 1.00 eq, HCl) in IPA (480 mL) was added DIEA (94.6 g, 732 mmol, 127 mL, 4.00 eq) at 20° C., then was added 3,5-dichloropyridazine (30.0 g, 201 mmol, 1.10 eq) at 20° C. The reaction mixture was stirred at 70° C. for 12 hrs. The reaction mixture was quenched with H₂O (1.00 L), then extracted with DCM (500 mL*3). The organic layer was washed with brine (500 mL*2), dried over Na₂SO₄, filtered and concentrated to give the product. The residue was purified by column chromatography (SiO₂) with a gradient 30-100% ethyl acetate in petroleum ether. Methyl 1-(6-chloropyridazin-4-yl)-4-(5-methylisoxazol-3-yl)piperidine-4-carboxylate (5.10 g, 7.92 mmol, 4.16% yield, 52.3% purity) was obtained as a brown solid. LCMS: C₁₅H₁₇ClN₄O₃ requires 336.1, found: m/z=337.1 [M+H]⁺.

### Step 4: Synthesis of methyl 1-(6-(2-methoxyphenyl)43yridazine-4-yl)-4-(5-methylisoxazol-3-yl)piperidine-4-carboxylate

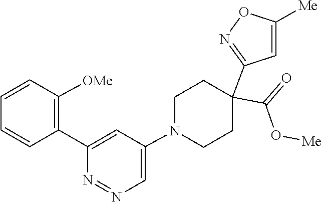 [SMILES: COC(=O)C1(c2cc(C)on2)CCN(c2cnnc(-c3ccccc3OC)c2)CC1]

A mixture of methyl 1-(6-chloropyridazin-4-yl)-4-(5-methylisoxazol-3-yl)piperidine-4-carboxylate (2.54 g, 7.54 mmol, 1.00 eq), (2-methoxyphenyl)boronic acid (2.29 g, 15.0 mmol, 2.00 eq), K₂CO₃ (3.13 g, 22.6 mmol, 3.00 eq), RuPhos Pd G3 (630 mg, 754 μmol, 0.10 eq) in dioxane (127 mL) and H₂O (12.7 mL) was degassed and purged with N₂ for 3 times at 25° C., and then the mixture was stirred at 90° C. for 12 hrs under N₂ atmosphere. Then the mixture was poured into H₂O (150 mL), extracted with ethyl acetate (150 mL*3). The combined organic phase was washed with brine (150 mL), dried over Na₂SO₄, filtered and concentrated under vacuum. The residue was purified by RP-FC with a gradient of 25-55% MeCN in H₂O. The eluent was concentrated in vacuum. The aqueous phase was lyophilized to give the product. Methyl 1-(6-(2-methoxyphenyl)pyridazine-4-yl)-4-(5-methylisoxazol-3-yl)piperidine-4-carboxylate (1.90 g, 4.50 mmol, 35.9% yield, 96.6% purity) was obtained as a light yellow solid. LCMS: C₂₂H₂₄N₄O₄ requires 408.2. found: m/z=409.1 [M+H]⁺.

### Step 5: Synthesis of Title Compound

To a solution of methyl 1-(6-(2-methoxyphenyl)pyridazine-4-yl)-4-(5-methylisoxazol-3-yl)piperidine-4-carboxylate (1.60 g, 3.79 mmol, 1.00 eq) in DCM (50.0 mL) was added BBr₃ (2 M, 3.79 mL, 2.00 eq) at 0° C. The reaction was stirred at 15° C. for 3 hrs. The resulting mixture was quenched by addition of HCl solution (1 N, 5.00 mL). The resulting mixture was extracted with EtOAc (10.0 mL*3), dried over Na₂SO₄, concentrated under reduced pressure to give methyl 1-(6-(2-hydroxyphenyl)pyridazine-4-yl)-4-(5-methylisoxazol-3-yl)piperidine-4-carboxylate (3.80 g, crude) was as a light yellow solid. Then, to a solution of methyl 1-(6-(2-hydroxyphenyl)pyridazine-4-yl)-4-(5-methylisoxazol-3-yl)piperidine-4-carboxylate (3.10 g, 2.33 mmol, 1.00 eq) in THF (30.0 mL) and H₂O (30.0 mL) was added LiOH·H₂O (977 mg, 23.3 mmol, 10.0 eq), and the mixture was stirred at 15° C. for 4 hrs. The resulting mixture was quenched by addition of HCl solution (1 N, 30.0 mL), then concentrated under vacuum. This was purified by RP-FC with a gradient of 1-31% MeCN in H₂O. The residue was dissolved with CAN (5.00 mL), H₂O (5.00 mL), concentrated under vacuum to removed CAN, then concentrated under lyophilization. 1-(6-(2-hydroxyphenyl)pyridazine-4-yl)-4-(5-methylisoxazol-3-yl)piperidine-4-carboxylic acid (444 mg, 1.13 mmol, 30.5% yield, 96.5% purity) was obtained as a white solid. LCMS: C₂₀H₂₀N₄O₄ requires 380.1. found: m/z=381.1 [M+H]⁺. ¹H NMR: (400 MHz, DMSO-d₆) δ 9.01-8.95 (m, 1H), 7.98-7.81 (m, 1H), 7.56 (d, J=2.8 Hz, 1H), 7.42-7.33 (m, 1H), 7.02-6.94 (m, 2H), 6.45 (s, 1H), 3.79-3.67 (m, 4H), 2.39-2.31 (m, 2H), 2.24-2.15 (m, 5H).

### Intermediate 2

### 1-(6-(2-hydroxyphenyl)pyridazin-4-yl)-4-(2-methoxyphenyl)piperidine-4-carboxylic acid

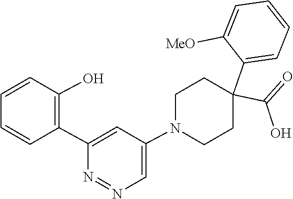 [SMILES: COc1ccccc1C1(C(=O)O)CCN(c2cnnc(-c3ccccc3O)c2)CC1]

### Step 1: Synthesis of 1-(tert-butyl) 4-methyl 4-(2-methoxyphenyl)piperidine-1,4-dicarboxylate

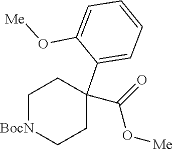 [SMILES: COC(=O)C1(c2ccccc2OC)CCN(C(=O)OC(C)(C)C)CC1]

This intermediate was synthesized according to Step 1 of Intermediate 1 using methyl 2-(2-methoxyphenyl)acetate. 1-(tert-butyl) 4-methyl 4-(2-methoxyphenyl)piperidine-1,4-dicarboxylate (2.90 g, 5.69 mmol, 4.60% yield, 68.6% purity) was a yellow solid. LCMS: C₁₉H₂₇NO₅ requires 349.2. found: m/z=250.1 [M−100+H]⁺.

### Step 2: Synthesis of methyl 4-(2-methoxyphenyl)piperidine-4-carboxylate

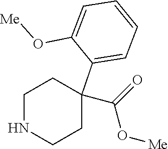 [SMILES: COC(=O)C1(c2ccccc2OC)CCNCC1]

This intermediate was synthesized according to Step 2 of Intermediate 1. Methyl 4-(2-methoxyphenyl)piperidine-4-carboxylate (2.52 g, crude, HCl) was obtained as brown oil. LCMS: C₁₄H₁₉NO₃ requires 249.1. found: m/z=250.1 [M+H]⁺.

### Step 3: Synthesis of methyl 1-(6-chloropyridazin-4-yl)-4-(2-methoxyphenyl)piperidine-4-carboxylate

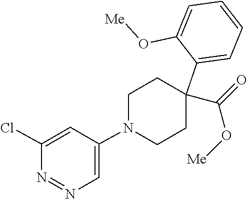 [SMILES: COC(=O)C1(c2ccccc2OC)CCN(c2cnnc(Cl)c2)CC1]

This intermediate was synthesized according to Step 3 of Intermediate 1 using methyl 4-(2-methoxyphenyl)piperidine-4-carboxylate. Methyl 1-(6-chloropyridazin-4-yl)-4-(2-methoxyphenyl)piperidine-4-carboxylate (2.05 g, 5.51 mmol, 84.5% yield, 97.3% purity) was obtained as yellow oil. LCMS: C₁₈H₂₀ClN₃O₃ requires 361.1. found: m/z=362.2 [M+H]⁺.

### Step 4: Synthesis of methyl 1-(6-(2-hydroxyphenyl)pyridazin-4-yl)-4-(2-methoxyphenyl)piperidine-4-carboxylate

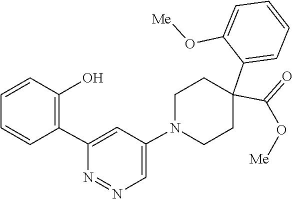 [SMILES: COC(=O)C1(c2ccccc2OC)CCN(c2cnnc(-c3ccccc3O)c2)CC1]

This intermediate was synthesized (2.5 g, crude) according to Step 4 of Intermediate 1 using methyl 1-(6-chloropyridazin-4-yl)-4-(2-methoxyphenyl)piperidine-4-carboxylate and (2-hydroxyphenyl)boronic acid. LCMS: C₂₄H₂₅N₃O₄ requires 419.2. found: m/z=420.2 [M+H]⁺.

Following basic hydrolysis, the title compound was obtained as an off-white solid (1.44 g, 3.52 mmol, 73.9% yield, 99.2% purity). ¹H NMR: (400 MHz, DMSO-d₆) δ 8.94 (d, J=2.8 Hz, 1H), 8.10 (dd, J=1.6, 8.4 Hz, 1H), 7.54 (d, J=2.8 Hz, 1H), 7.36-7.30 (m, 2H), 7.29-7.23 (m, 1H), 7.01 (d, J=7.6 Hz, 1H), 6.97-6.89 (m, 3H), 3.99-3.91 (m, 2H), 3.74 (s, 3H), 3.61-3.51 (m, 2H), 2.38 (br d, J=14.0 Hz, 2H), 2.10-2.00 (m, 2H).

### Intermediate 4

### 1-(6-(2-hydroxyphenyl)pyridazin-4-yl)-4-(tetrahydro-2H-pyran-4-yl)piperidine-4-carboxylic acid

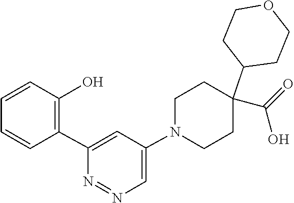 [SMILES: O=C(O)C1(C2CCOCC2)CCN(c2cnnc(-c3ccccc3O)c2)CC1]

### Step 1: Synthesis of ethyl 4-(tetrahydro-2H-pyran-4-yl)piperidine-4-carboxylate

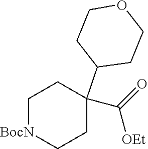 [SMILES: CCOC(=O)C1(C2CCOCC2)CCN(C(=O)OC(C)(C)C)CC1]

To a solution of DIPA (4.71 g, 46.6 mmol, 6.58 mL, 1.20 eq) in THF (50.0 mL) was added n-BuLi (2.50 M, 18.7 mL, 1.20 eq) at −10° C., and stirred at −10° C. for 0.5 hr. Then, ethyl piperidine-4-carboxylate (10.0 g, 38.9 mmol, 1.00 eq) in THF (10.0 mL) was added drop wise to the reaction mixture at −10° C., and stirred at −10° C. for 2 hr. A solution of 4-iodotetrahydro-2H-pyran (9.06 g, 42.8 mmol, 1.10 eq) in THF (10.0 mL) was added to the mixture at −10° C. and stirred at −10° C. for 1 hr. The mixture was warmed to 25° C. and stirred at 25° C. for 12 hrs. The reaction mixture was poured into saturated ice NH₄Cl solution (500 mL) at 0° C., the aqueous phase was extracted with ethyl acetate (500 mL*2). The combined organic phase was washed with H₂O (500 mL), brine (500 mL), dried with anhydrous Na₂SO₄, filtered and concentrated in vacuum to give a crude product.

The residue was purified by column chromatography (SiO₂) with a gradient of 1-20% ethyl acetate in petroleum ether. Ethyl 4-(tetrahydro-2H-pyran-4-yl)piperidine-4-carboxylate (8.90 g, crude) was obtained as a yellow oil. ¹H NMR: (400 MHz, MeOD) δ 4.21 (q, J=7.2 Hz, 2H), 4.06-3.88 (m, 4H), 3.41-3.32 (m, 2H), 2.74 (br s, 2H), 2.14 (br d, J=12.4 Hz, 2H), 1.70-1.62 (m, 1H), 1.61-1.55 (m, 2H), 1.46-1.44 (m, 9H), 1.43-1.32 (m, 4H), 1.29 (t, J=7.2 Hz, 3H)

### Step 2: Synthesis of ethyl 4-(tetrahydro-2H-pyran-4-yl)piperidine-4-carboxylate

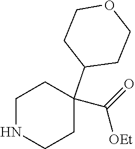 [SMILES: CCOC(=O)C1(C2CCOCC2)CCNCC1]

This intermediate was synthesized according to Step 2 of Intermediate 1. Ethyl 4-(tetrahydro-2H-pyran-4-yl)piperidine-4-carboxylate (6.00 g, 21.6 mmol, 82.9% yield, 100% purity, HCl) was obtained as a light yellow solid. LCMS: C₁₃H₂₃NO₃ requires 241.2. found: m/z=242.2 [M+H]⁺.

### Step 3: Synthesis of ethyl 1-(6-chloropyridazin-4-yl)-4-(tetrahydro-2H-pyran-4-yl)piperidine-4-carboxylate

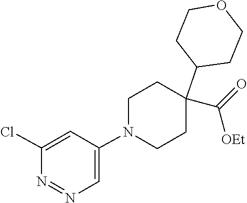 [SMILES: CCOC(=O)C1(C2CCOCC2)CCN(c2cnnc(Cl)c2)CC1]

This intermediate was synthesized (5.40 g, 15.0 mmol, 69.4% yield, 98.2% purity) according to Step 3 of Intermediate 1 using ethyl 4-(tetrahydro-2H-pyran-4-yl)piperidine-4-carboxylate. LCMS: C₁₇H₂₄ClN₃O₃ requires 353.2. found: m/z=354.1 [M+H]⁺.

### Step 4: Synthesis of methyl 1-(6-(2-hydroxyphenyl)pyridazin-4-yl)-4-(2-methoxyphenyl)piperidine-4-carboxylate

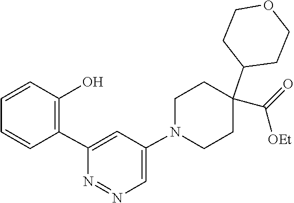 [SMILES: CCOC(=O)C1(C2CCOCC2)CCN(c2cnnc(-c3ccccc3O)c2)CC1]

This intermediate was synthesized (4.50 g, 10.6 mmol, 78.1% yield, 97.1% purity) according to Step 4 of Intermediate 1 using ethyl 1-(6-(2-hydroxyphenyl)pyridazin-4-yl)-4-(tetrahydro-2H-pyran-4-yl)piperidine-4-carboxylate and (2-hydroxyphenyl)boronic acid. LCMS: C₂₃H₂₉N₃O₄ requires 411.2. found: m/z=412.2 [M+H]⁺.

Following basic hydrolysis, the title compound was obtained as a beige solid (2.43 g, 6.28 mmol, 88.7% yield, 99.1% purity). LCMS: C₂₁H₂₅N₃O₄ requires 383.2. found: m/z=384.1 [M+H]⁺. ¹H NMR: (400 MHz, DMSO-d₆) δ 13.96-13.48 (m, 1H), 8.93 (d, J=2.8 Hz, 1H), 8.10 (d, J=8.0 Hz, 1H), 7.53 (d, J=2.8 Hz, 1H), 7.32 (t, J=7.6 Hz, 1H), 6.96-6.87 (m, 2H), 4.22 (br d, J=13.6 Hz, 2H), 3.87 (br dd, J=3.2, 10.8 Hz, 2H), 3.22 (br t, J=11.2 Hz, 2H), 2.96 (br t, J=12.4 Hz, 2H), 2.10 (br d, J=13.2 Hz, 2H), 1.68-1.59 (m, 1H), 1.55-1.42 (m, 4H), 1.36-1.22 (m, 2H)

### Intermediate 5

### 1-(6-(2-hydroxyphenyl)pyridazin-4-yl)-4-(3-methyl-1H-pyrazol-1-yl)piperidine-4-carboxylic acid

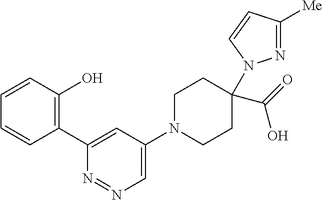 [SMILES: Cc1ccn(C2(C(=O)O)CCN(c3cnnc(-c4ccccc4O)c3)CC2)n1]

### Step 1: Synthesis of di-tert-butyl 1-(1-benzyl-4-(ethoxycarbonyl)piperidin-4-yl)hydrazine-1,2-dicarboxylate

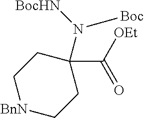 [SMILES: CCOC(=O)C1(N(NC(=O)OC(C)(C)C)C(=O)OC(C)(C)C)CCN(Cc2ccccc2)CC1]

To the solution of DIPA (15.5 g, 153 mmol, 21.6 mL, 1.40 eq) in THF (270 mL) was added n-BuLi (2.5 M, 60.9 mL, 1.39 eq) at −65° C. under N₂. The reaction mixture was stirred at −65° C. for 1 hr. Then ethyl 1-benzylpiperidine-4-carboxylate (27.0 g, 109 mmol, 1.00 eq) was added drop wise to the reaction mixture at −65° C. and stirred at −65° C. for 1 hr. 1,2-Bis(1,1-dimethylethyl)-(1E)-1,2-diazenedicarboxylate (27.5 g, 120 mmol, 1.10 eq) in THF (50.0 mL) was added to the mixture at −65° C. The reaction mixture was warmed to 25° C. and stirred at 25° C. for 12 hrs under N₂. The reaction mixture was poured into saturated NH₄Cl solution (1.50 L) and extracted with ethyl acetate (600 mL*3). The combined organic phase was washed with brine (1.50 L), dried with anhydrous Na₂SO₄, filtered and concentrated in vacuum. The residue was purified by RP-FC and purified with a gradient of 25-46% MeCN in H₂O. The eluent was concentrated in vacuum to remove acetonitrile and adjusted to pH=7 with saturated NaHCO₃ solution. The aqueous phase was extracted with ethyl acetate (1.00 L*3), dried with anhydrous Na₂SO₄, filtered and concentrated in vacuum to give di-tert-butyl 1-(1-benzyl-4-(ethoxycarbonyl)piperidin-4-yl)hydrazine-1,2-dicarboxylate (48.8 g, 97.1 mmol, 88.9% yield) as a yellow solid. LCMS: C₂₅H₃₉N₃O₆ requires 477.3. found: m/z=478.1 [M+H]⁺.

### Step 2: Synthesis of ethyl 1-benzyl-4-hydrazineylpiperidine-4-carboxylate

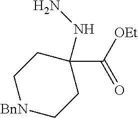 [SMILES: CCOC(=O)C1(NN)CCN(Cc2ccccc2)CC1]

To a solution of di-tert-butyl 1-(1-benzyl-4-(ethoxycarbonyl)piperidin-4-yl)hydrazine-1,2-dicarboxylate (48.8 g, 102 mmol, 1.00 eq) in EtOAc (50.0 mL) was added HCl/EtOAc (2 M, 500 mL, 9.79 eq) at 25° C. The mixture was heated to 40° C. and stirred at 40° C. for 12 hrs. The reaction mixture was concentrated under vacuum to give ethyl 1-benzyl-4-hydrazineylpiperidine-4-carboxylate (36.0 g, crude, HCl) as a white solid. LCMS: C₁₅H₂₃N₃O₂ requires 277.2. found: m/z=278.1 [M+H]⁺.

### Step 3: Synthesis of ethyl 1-benzyl-4-(3-methyl-1H-pyrazol-1-yl)piperidine-4-carboxylate

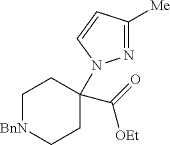 [SMILES: CCOC(=O)C1(n2ccc(C)n2)CCN(Cc2ccccc2)CC1]

A mixture of ethyl 1-benzyl-4-hydrazineylpiperidine-4-carboxylate (39.0 g, 141 mmol, 1.00 eq) and 4,4-dimethoxybutan-2-one (18.6 g, 141 mmol, 18.7 mL, 1.00 eq) in EtOH (195 mL) and AcOH (19.5 mL) was degassed and purged with N₂ for 3 times, and then the mixture was stirred at 50° C. for 2 hrs under N₂. The reaction mixture was concentrated under vacuum. The residue was purified by RP-FC with a gradient of 10-40% MeCN in H₂O. The eluent was concentrated in vacuum to remove acetonitrile and H₂O, the residual aqueous was lyophilized to give ethyl 1-benzyl-4-(3-methyl-1H-pyrazol-1-yl)piperidine-4-carboxylate (22.0 g, 64.4 mmol, 45.8% yield) as a yellow solid. LCMS: C₁₉H₂₅N₃O₂ requires 327.2. found: m/z=328.1 [M+H]⁺.

### Step 4: Synthesis of ethyl 4-(3-methyl-1H-pyrazol-1-yl)piperidine-4-carboxylate

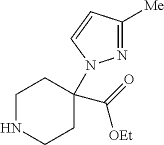 [SMILES: CCOC(=O)C1(n2ccc(C)n2)CCNCC1]

To a solution of ethyl 1-benzyl-4-(3-methyl-1H-pyrazol-1-yl)piperidine-4-carboxylate (22.0 g, 67.2 mmol, 1.00 eq) in MeOH (110 mL) was added Pd/C (2.22 g, 2.08 mmol, 10% purity, 0.031 eq) under N₂. The suspension was degassed under vacuum and purged with H₂ three times. The mixture was stirred under H₂ (50 psi) at 25° C. for 12 hrs. The reaction mixture was filtered and the filter was concentrated to give ethyl 4-(3-methyl-1H-pyrazol-1-yl)piperidine-4-carboxylate (15.6 g, 64.8 mmol, 96.5% yield) as a yellow solid. ¹H NMR: (400 MHz, DMSO-d₆) δ 8.69 (s, 1H), 7.90 (d, J=2.4 Hz, 1H), 6.18 (d, J=2.4 Hz, 1H), 4.08 (q, J=7.2 Hz, 2H), 3.24 (d, J=13.2 Hz, 2H), 2.87 (s, 2H), 2.68 (d, J=15.2 Hz, 2H), 2.49-2.40 (m, 2H), 2.17 (s, 3H), 1.10 (t, J=7.2 Hz, 3H).

### Step 5: Synthesis of ethyl 1-(6-chloropyridazin-4-yl)-4-(3-methyl-1H-pyrazol-1-yl)piperidine-4-carboxylate

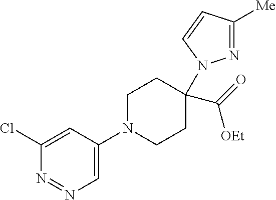 [SMILES: CCOC(=O)C1(n2ccc(C)n2)CCN(c2cnnc(Cl)c2)CC1]

This intermediate was synthesized (12.5 g, 35.5 mmol, 53.9% yield) according to Step 3 of Intermediate 1 using ethyl 4-(3-methyl-1H-pyrazol-1-yl)piperidine-4-carboxylate. LCMS: C₁₆H₂₀ClN₅O₂ requires 349.1. found: m/z=350.0 [M+H]⁺.

### Step 6: Synthesis of ethyl 1-(6-(2-hydroxyphenyl)pyridazin-4-yl)-4-(3-methyl-1H-pyrazol-1-yl)piperidine-4-carboxylate

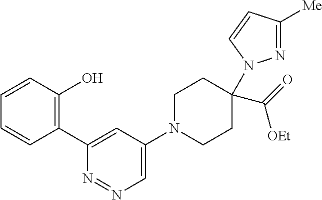 [SMILES: CCOC(=O)C1(n2ccc(C)n2)CCN(c2cnnc(-c3ccccc3O)c2)CC1]

This intermediate was synthesized (8.50 g, 16.7 mmol, 83.4% yield) according to Step 4 of Intermediate 1 using ethyl 1-(6-chloropyridazin-4-yl)-4-(3-methyl-1H-pyrazol-1-yl)piperidine-4-carboxylate and (2-hydroxyphenyl)boronic acid. LCMS: C₂₂H₂₅N₅O₃ requires 407.2. found: m/z=408.2 [M+H]⁺.

Following basic hydrolysis, the title compound was obtained as a light brown solid (1.07 g, 2.75 mmol, 37.3% yield, 97.4% purity). LCMS: C₂₀H₂₁N₅O₃ requires 379.4. found: m/z=380.1 [M+H]⁺. ¹H NMR: (400 MHz, MeOD) δ 8.84 (d, J=2.8 Hz, 1H), 7.81-7.70 (m, 2H), 7.50 (d, J=3.2 Hz, 1H), 7.43-7.35 (m, 1H), 7.07-6.94 (m, 2H), 6.13 (d, J=2.2 Hz, 1H), 3.98-3.85 (m, 2H), 3.77-3.62 (m, 2H), 2.66-2.55 (m, 4H), 2.25 (s, 3H)

### Intermediate 6

### 1-(6-(2-hydroxyphenyl)pyridazin-4-yl)-4-(pyridin-4-yl)piperidine-4-carboxylic acid

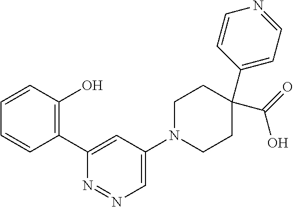 [SMILES: O=C(O)C1(c2ccncc2)CCN(c2cnnc(-c3ccccc3O)c2)CC1]

### Step 1: Synthesis of 1-(tert-butyl) 4-methyl 4-(pyridin-4-yl)piperidine-1,4-dicarboxylate

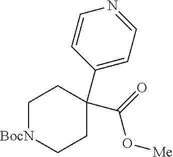 [SMILES: COC(=O)C1(c2ccncc2)CCN(C(=O)OC(C)(C)C)CC1]

This intermediate was synthesized (9.20 g, 25.4 mmol, 9.84% yield, 88.4% purity) according to Step 1 of Intermediate 1 using methyl 2-(4-fluorophenyl)acetate. LCMS: C₁₇H₂₄N₂O₄ requires 320.2. found: m/z=321.3 [M+H]⁺.

### Step 2: Synthesis of methyl 4-(pyridin-4-yl)piperidine-4-carboxylate

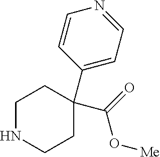 [SMILES: COC(=O)C1(c2ccncc2)CCNCC1]

This intermediate was synthesized (9.20 g, crude, HCl) according to Step 2 of Intermediate 1. LCMS: C₁₂H₁₆N₂O₂ requires 220.1. found: m/z=221.1 [M+H]⁺.

### Step 3: Synthesis of methyl 1-(6-chloropyridazin-4-yl)-4-(pyridin-4-yl)piperidine-4-carboxylate

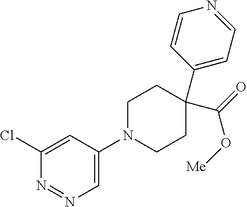 [SMILES: COC(=O)C1(c2ccncc2)CCN(c2cnnc(Cl)c2)CC1]

This intermediate was synthesized (6.67 g, 19.8 mmol, 62.0% yield, 98.8% purity) according to Step 3 of Intermediate 1 using methyl 4-(pyridin-4-yl)piperidine-4-carboxylate. LCMS: C₁₆H₁₇ClN₄O₂ requires 332.1. found: m/z=333.2 [M+H]⁺.

### Step 4: Synthesis of methyl 1-(6-(2-hydroxyphenyl)pyridazin-4-yl)-4-(pyridin-4-yl)piperidine-4-carboxylate

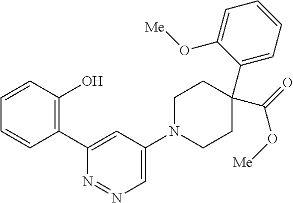 [SMILES: COC(=O)C1(c2ccccc2OC)CCN(c2cnnc(-c3ccccc3O)c2)CC1]

This intermediate was synthesized (4.20 g, 10.6 mmol, 63.9% yield, 98.2% purity) according to Step 4 of Intermediate 1 using methyl 1-(6-chloropyridazin-4-yl)-4-(pyridin-4-yl)piperidine-4-carboxylate and (2-hydroxyphenyl)boronic acid. LCMS: C₂₂H₂₂N₄O₃ requires 390.2. found: m/z=391.2 [M+H]⁺.

Following basic hydrolysis, the title compound (1.88 g, 4.58 mmol, 96.6% yield, 96.2% purity, Li salt) was obtained as a brown solid. LCMS: C₂₁H₂₀N₄O₃ requires 376.2. found: m/z=377.2. ¹H NMR: (400 MHz, D₂O)δ 8.56 (d, J=3.2 Hz, 1H), 8.32 (d, J=6.4 Hz, 2H), 7.31-7.25 (m, 4H), 7.19-7.10 (m, 1H), 6.68 (d, J=8.0 Hz, 1H), 6.57 (t, J=7.2 Hz, 1H), 3.68-3.59 (m, 2H), 3.25-3.14 (m, 2H), 2.32 (d, J=13.6 Hz, 2H), 1.68-1.78 (m, 2H).

### Intermediate 7

### 1-(6-(2-hydroxyphenyl)pyridazin-4-yl)-4-(3-methoxyphenyl)piperidine-4-carboxylic acid

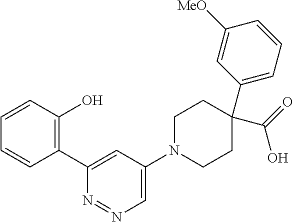 [SMILES: COc1cccc(C2(C(=O)O)CCN(c3cnnc(-c4ccccc4O)c3)CC2)c1]

### Step 1: Synthesis of 1-(tert-butyl) 4-methyl 4-(3-methoxyphenyl)piperidine-1,4-dicarboxylate

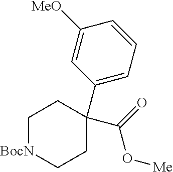 [SMILES: COC(=O)C1(c2cccc(OC)c2)CCN(C(=O)OC(C)(C)C)CC1]

This intermediate was synthesized (7.70 g, 11.0 mmol, 6.62% yield, 50.0% purity) according to Step 1 of Intermediate 1 using methyl 2-(3-methoxyphenyl)acetate. LCMS: C₁₉H₂₇NO₅ requires 349.2. found: m/z=250.1 [M−100+H]⁺.

### Step 2: Synthesis of methyl 4-(3-methoxyphenyl)piperidine-4-carboxylate

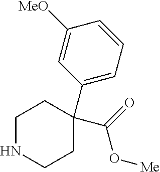 [SMILES: COC(=O)C1(c2cccc(OC)c2)CCNCC1]

This intermediate was synthesized (7.40 g, crude, HCl) according to Step 2 of Intermediate 1. LCMS: C₁₄H₁₉NO₃ requires 249.1. found: m/z=250.1 [M+H]⁺.

### Step 3: Synthesis of methyl 1-(6-chloropyridazin-4-yl)-4-(3-methoxyphenyl)piperidine-4-carboxylate

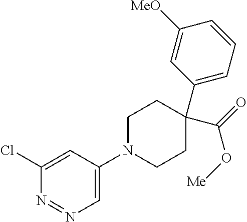 [SMILES: COC(=O)C1(c2cccc(OC)c2)CCN(c2cnnc(Cl)c2)CC1]

This intermediate was synthesized (5.90 g, 16.0 mmol, 57.3% yield, 98.3% purity) according to Step 3 of Intermediate 1 using methyl 4-(3-methoxyphenyl)piperidine-4-carboxylate. LCMS: C₁₈H₂₀ClN₃O₃ requires 361.1. found: m/z=362.2 [M+H]⁺.

### Step 4: Synthesis of methyl 1-(6-(2-hydroxyphenyl)pyridazin-4-yl)-4-(3-methoxyphenyl)piperidine-4-carboxylate

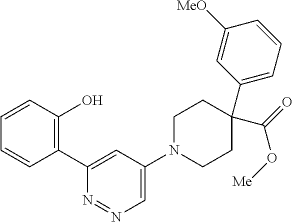 [SMILES: COC(=O)C1(c2cccc(OC)c2)CCN(c2cnnc(-c3ccccc3O)c2)CC1]

This intermediate was synthesized (3.20 g, 7.40 mmol, 59.5% yield, 97% purity) according to Step 4 of Intermediate 8 using methyl 1-(6-chloropyridazin-4-yl)-4-(3-methoxyphenyl)piperidine-4-carboxylate and (2-hydroxyphenyl)boronic acid. LCMS: C₂₄H₂₅N₃O₄ requires 419.2. found: m/z=420.1 [M+H]⁺.

Following basic hydrolysis, the title compound (1.04 g, 2.56 mmol, 39.8% yield, 99.9% purity) was obtained as an off-white solid. LCMS: C₂₃H₂₃N₃O₄ requires 405.2. found: m/z=406.2 [M+H]⁺. ¹H NMR: (400 MHz, DMSO-d₆) δ 8.86 (d, J=3.20 Hz, 1H), 7.58 (m, 1H), 7.43-7.51 (m, 2H), 7.30 (t, J=8.00 Hz, 1H), 6.98-7.10 (m, 4H), 6.87 (dd, J=8.40, 2.40 Hz, 1H), 4.19-4.43 (m, 2H), 3.80 (s, 3H), 3.60 (s, 2H), 2.73 (d, J=13.2 Hz, 2H), 2.06-2.20 (m, 2H).

### Intermediate 8

### 4-(4-fluorophenyl)-1-(6-(2-hydroxyphenyl)pyridazin-4-yl)piperidine-4-carboxylic acid

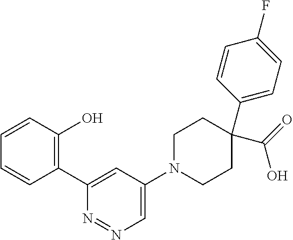 [SMILES: O=C(O)C1(c2ccc(F)cc2)CCN(c2cnnc(-c3ccccc3O)c2)CC1]

### Step 1: Synthesis of 1-(tert-butyl) 4-methyl 4-(4-fluorophenyl)piperidine-1,4-dicarboxylate

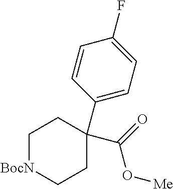 [SMILES: COC(=O)C1(c2ccc(F)cc2)CCN(C(=O)OC(C)(C)C)CC1]

This intermediate was synthesized (8.36 g, crude) according to Step 1 of Intermediate 1 using methyl 2-(4-fluorophenyl)acetate. LCMS: C₁₈H₂₄FNO₄ requires 337.2. found: m/z=282.2 [M−56+H]⁺.

### Step 2: Synthesis of methyl 4-(4-fluorophenyl)piperidine-4-carboxylate

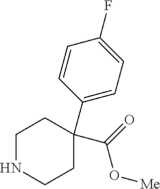 [SMILES: COC(=O)C1(c2ccc(F)cc2)CCNCC1]

This intermediate was synthesized (7.64 g, crude, HCl) according to Step 2 of Intermediate 1. LCMS: C₁₃H₁₆FNO₂ requires 237.1. found: m/z=238.2 [M+H]⁺.

### Step 3: Synthesis of methyl 1-(6-chloropyridazin-4-yl)-4-(4-fluorophenyl)piperidine-4-carboxylate

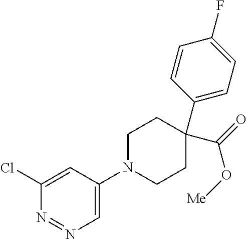 [SMILES: COC(=O)C1(c2ccc(F)cc2)CCN(c2cnnc(Cl)c2)CC1]

This intermediate was synthesized (2.61 g, 7.31 mmol, 41.0% yield, 98.0% purity) according to Step 3 of Intermediate 1 using methyl 4-(4-fluorophenyl)piperidine-4-carboxylate. LCMS: C₁₇H₁₇C₁FN₃O₂ requires 349.1. found: m/z=350.2 [M+H]⁺.

### Step 4: Synthesis of methyl 4-(4-fluorophenyl)-1-(6-(2-hydroxyphenyl)pyridazin-4-yl)piperidine-4-carboxylate

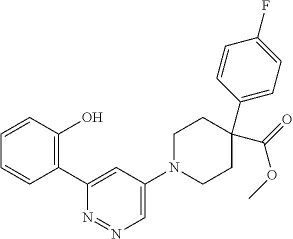 [SMILES: COC(=O)C1(c2ccc(F)cc2)CCN(c2cnnc(-c3ccccc3O)c2)CC1]

To a solution of methyl 1-(6-chloropyridazin-4-yl)-4-(4-fluorophenyl)piperidine-4-carboxylate (2.41 g, 6.75 mmol, 1.00 eq), (2-hydroxyphenyl)boronic acid (1.86 g, 13.5 mmol, 2.00 eq) and K₂CO₃ (2.80 g, 20.3 mmol, 3.00 eq) in dioxane (24.1 mL) and H₂O (4.82 mL) was added Pd(dppf)Cl₂ (494 mg, 675 μmol, 0.10 eq) under N₂ atmosphere. The mixture was stirred at 110° C. for 12 hrs. The mixture was poured into H₂O (100 mL) and extracted with ethyl acetate (100 mL*3). The combined organic phase was washed with brine (100 mL), dried over Na₂SO₄, filtered and concentrated under vacuum. The crude product was purified by MPLC (SiO2) with a gradient of 10-50% Petroleum ether/Ethyl acetate (751 mg, 1.80 mmol, 26.6% yield, 97.6% purity). LCMS: C₂₃H₂₂FN₃O₃ requires 407.2. found: m/z=408.2 [M+H]⁺.

Following basic hydrolysis, the title compound (1.14 g, 2.62 mmol, 79.2% yield, 98.8% purity, HCl) was obtained as a white solid. LCMS: C₂₂H₂₀FN₃O₃ requires 393.1. found: m/z=394.2 [M+H]⁺. ¹H NMR: (400 MHz, DMSO-d₆) δ 13.43-12.64 (m, 1H), 9.01 (d, J=3.2 Hz, 1H), 7.57-7.52 (m, 2H), 7.49-7.43 (m, 3H), 7.24-7.19 (m, 2H), 7.13 (d, J=8.0 Hz, 1H), 7.05-6.99 (m, 1H), 4.45-4.21 (m, 2H), 4.04-4.00 (m, 1H), 3.51-3.43 (m, 2H), 2.57 (br d, J=13.6 Hz, 2H), 2.05-1.96 (m, 2H). ¹⁹F NMR: (400 MHz, DMSO-d₆) δ −115.561.

### Intermediate 9

### 4-(2-fluorophenyl)-1-(6-(2-hydroxyphenyl)pyridazin-4-yl)piperidine-4-carboxylic acid

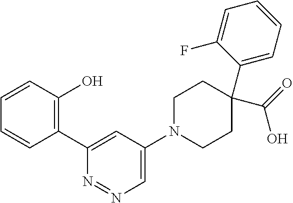 [SMILES: O=C(O)C1(c2ccccc2F)CCN(c2cnnc(-c3ccccc3O)c2)CC1]

### Step 1: Synthesis of 1-(tert-butyl) 4-methyl 4-(2-fluorophenyl)piperidine-1,4-dicarboxylate

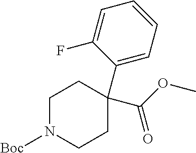 [SMILES: COC(=O)C1(c2ccccc2F)CCN(C(=O)OC(C)(C)C)CC1]

To a solution of methyl 2-(2-fluorophenyl)acetate (28.0 g, 164 mmol) in DMF (560 mL) was added NaH (16.4 g, 411 mmol, 60.0% in mineral oil) at 0-5° C. under N₂, the mixture was stirred at 0-5° C. for 1 hr. Then, tert-butyl bis(2-chloroethyl)carbamate (51.8 g, 214 mmol) was added, the mixture was heated to 60° C. and stirred until complete as judged by LCMS. Once complete, the reaction mixture was quenched with saturated NH₄Cl solution (300 mL) at 0° C. The mixture was poured into H₂O (500 mL) and extracted with EtOAc (500 mL×3). The combined organic phase was washed with H₂O (500 mL×3), dried over anhydrous Na₂SO₄, filtered, and concentrated under vacuum to afford the crude product (54.0 g) as a yellow oil which was taken forward without any further purification. LCMS C₉H₉FO₂ requires 337.2, found 282.2 [M−56+H]⁺.

### Step 2: Synthesis of methyl 4-(2-fluorophenyl)piperidine-4-carboxylate

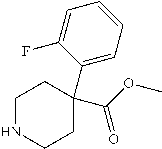 [SMILES: COC(=O)C1(c2ccccc2F)CCNCC1]

To a solution of 1-(tert-butyl) 4-methyl 4-(2-fluorophenyl)piperidine-1,4-dicarboxylate (54.0 g, 160 mmol) in EtOAc (200 mL) was added a 1:1 mixture of cone HCl in EtOAc (300 mL). The mixture was stirred at 25° C. until complete as judged by LCMS. Once complete, the reaction mixture was filtered and concentrated under reduced pressure to give a residue. The residue was adjusted to pH=8 with saturated sodium bicarbonate solution and extracted with DCM (100 mL). The aqueous phase was adjusted to pH=10 with saturated sodium carbonate solution and extracted with DCM (200 mL×3). The combined organic layers were dried over anhydrous Na₂SO₄, filtered, and concentrated under reduced pressure to give the crude product (16.0 g, HCl salt) as a yellow oil which was taken forward without any further purification. LCMS C₁₇H₁₇C₁FN₃O₂ requires 237.1, found m/z=238.2 [M+H]⁺.

### Step 3: Synthesis of methyl 1-(6-chloropyridazin-4-yl)-4-(2-fluorophenyl)piperidine-4-carboxylate

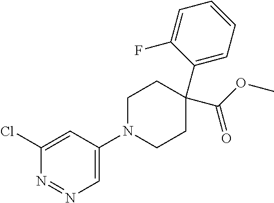 [SMILES: COC(=O)C1(c2ccccc2F)CCN(c2cnnc(Cl)c2)CC1]

To a solution of methyl 4-(2-fluorophenyl)piperidine-4-carboxylate (16.0 g, 67.4 mmol) in IPA (160 mL) was added DIPEA (46.9 mL, 269 mmol) and 3,5-dichloropyridazine (12.0 g, 80.9 mmol). The mixture was stirred at 25° C. until complete as judged by LCMS. Once complete, the reaction mixture was filtered and concentrated under reduced pressure to give a crude residue. The residue was dissolved in minimal DCM, poured into H₂O (200 mL) and then extracted with DCM (200 mL×3). The combined organic layer was dried over anhydrous Na₂SO₄, filtered, and concentrated to give the crude product. The crude residue was purified by flash column chromatography (petroleum ether in ethyl acetate: 15% to 50%) to afford the desired product as a yellow solid (9.45 g, 39% yield). LCMS C₁₇H₁₇C₁FN₃O₂ requires 349.1, found m/z=350.2 [M+H]⁺.

### Step 4: Synthesis of methyl 4-(2-fluorophenyl)-1-(6-(2-hydroxyphenyl)pyridazin-4-yl)piperidine-4-carboxylate

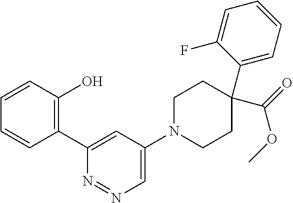 [SMILES: COC(=O)C1(c2ccccc2F)CCN(c2cnnc(-c3ccccc3O)c2)CC1]

To a solution of methyl 1-(6-chloropyridazin-4-yl)-4-(2-fluorophenyl)piperidine-4-carboxylate (7.40 g, 20.3 mmol) in 1,4-dioxane (75 mL) and H₂O (15 mL) was added K₂CO₃ (8.43 g, 60.9 mmol), Pd(dppf)Cl₂ (2.23 g, 3.05 mmol) and (2-hydroxyphenyl)boronic acid (5.61 g, 40.6 mmol) under N₂ atmosphere at 25° C. The mixture was then heated to 90° C. and allowed to stir until complete as judged by LCMS. Once complete, the reaction mixture was filtered through Celite (with EtOAc washing) and concentrated under reduced pressure to minimal solvent. The residue was poured into H₂O (200 mL) and was extracted with EtOAc (300 mL×3). The combined organic layer was dried over Na₂SO₄, filtered and concentrated to give a crude residue. The crude product was purified by flash column chromatography to afford the desired product as a yellow solid (2.75 g, 30.6% yield). LCMS C₂₃H₂₂FN₃O₃ requires m/z=407.2, found 408.3 [M+H]⁺.

Following basic hydrolysis, the title compound was obtained as a light-yellow solid (1.97 g, 71% yield). LCMS C₂₂H₂₀FN₃O₃ requires 393.2, found m/z=394.2 [M+H]⁺.

### Intermediate 10

### 1-(6-(2-hydroxyphenyl)pyridazin-4-yl)-4-(5-isopropylisoxazol-3-yl)piperidine-4-carboxylic acid

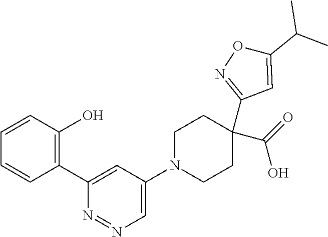 [SMILES: CC(C)c1cc(C2(C(=O)O)CCN(c3cnnc(-c4ccccc4O)c3)CC2)no1]

### Step 1: Synthesis of (5-isopropylisoxazol-3-yl)methanol

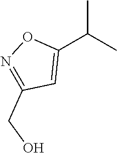

To a solution of methyl 5-isopropylisoxazole-3-carboxylate (25.0 g, 148 mmol) in THF (125 mL) was added LAH (2.5 M in THF, 118 mL) at 0° C. under N₂. The mixture was then allowed to warm and stirred at 25° C. under N₂ until complete as judged by LCMS. Once complete, the mixture was cooled to 0° C., followed by slow addition of 1 M HCl (800 mL) drop-wise under N₂ with stirring for 30 mins. The mixture was then extracted with MTBE (200 mL×3), washed with saturated NaHCO₃ aqueous solution (50 mL), dried over Na₂SO₄, filtered and concentrated under vacuum to afford the crude product as a yellow oil which was taken forward without any further purification (13.0 g). LCMS C₇H₁₁NO₂ requires 141.1, found 142.6 [M+H]⁺.

### Step 2: Synthesis of (5-isopropylisoxazol-3-yl)methyl methanesulfonate

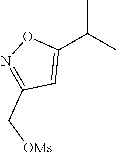

To a solution of (5-isopropylisoxazol-3-yl)methanol (20.1 g, 142 mmol) and TEA (39.6 mL, 285 mmol) in DCM (201 mL) was added MsCl (16.6 mL, 215 mmol) drop-wise at 0° C. The mixture was allowed to warm and stirred at 25° C. until complete as judged by LCMS. Once complete, the mixture was poured into ice-cold water (500 mL) and extracted with DCM (250 mL×3). The combined organic layers were washed with brine (100 mL), dried over Na₂SO₄, filtered and concentrated under vacuum to afford the crude product as a yellow oil which was taken forward without any further purification (28.1 g). LCMS C₈H₁₃NO₄S requires 219.1, found m/z=220.4 [M+H]⁺.

### Step 3: Synthesis of 2-(5-isopropylisoxazol-3-yl)acetonitrile

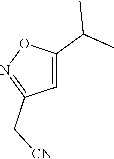

To a solution of (5-isopropylisoxazol-3-yl)methyl methanesulfonate (29.6 g, 135 mmol) in DMF (150 mL) was added NaCN (10.2 g, 208 mmol). The mixture was then heated to 60° C. and allowed to stir until complete as judged by LCMS. Once complete, the reaction mixture was diluted with saturated aqueous Na₂CO₃ solution (750 mL) and extracted with MTBE (250 mL×3). The organic layer was washed with brine (100 mL×2), dried over anhydrous Na₂SO₄, filtered and concentrated under vacuum to give a crude residue. The crude residue was purified by flash column chromatography (petroleum ether in ethyl acetate—5% to 20%) afford the desired product as a yellow oil (13.9 g, 69% yield). LCMS C₈H₁₀N₂O requires 150.1, found 151.2 [M+H]⁺.

### Step 4: tert-butyl 4-cyano-4-(5-isopropylisoxazol-3-yl)piperidine-1-carboxylate

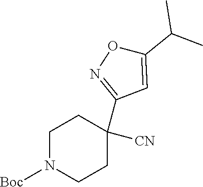 [SMILES: CC(C)c1cc(C2(C#N)CCN(C(=O)OC(C)(C)C)CC2)no1]

This intermediate was synthesized as described for Step 1 for Intermediate 9, using 2-(5-isopropylisoxazol-3-yl)acetonitrile in place of methyl 2-(2-fluorophenyl)acetate. LCMS C₁₇H₂₅N₃O₃ requires 319.2, found m/z=220.1 [M−Boc+H]⁺.

### Step 5: Synthesis of 4-(5-isopropylisoxazol-3-yl)piperidine-4-carbonitrile

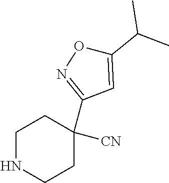 [SMILES: CC(C)c1cc(C2(C#N)CCNCC2)no1]

This intermediate was prepared as described in Step 2 for Intermediate 9, using tert-butyl 4-cyano-4-(5-isopropylisoxazol-3-yl)piperidine-1-carboxylate (Step 7) in place of 1-(tert-butyl) 4-methyl 4-(2-fluorophenyl)piperidine-1,4-dicarboxylate (9.65 g). LCMS C₁₂H₁₇N₃O requires 219.1, found m/z=220.1 [M+H]⁺.

### Step 6: Synthesis of 1-(6-chloropyridazin-4-yl)-4-(5-isopropylisoxazol-3-yl)piperidine-4-carbonitrile

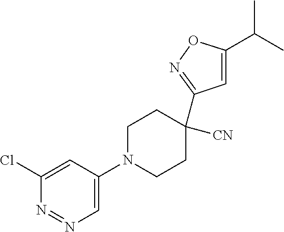 [SMILES: CC(C)c1cc(C2(C#N)CCN(c3cnnc(Cl)c3)CC2)no1]

This intermediate was prepared as described in Step 3 for Intermediate 9, using 4-(5-isopropylisoxazol-3-yl)piperidine-4-carbonitrile in place of 4-(2-fluorophenyl)piperidine-4-carboxylate (14.5 g). LCMS C₁₆H₁₈ClN₅O requires 331.1, found m/z=332.1 [M+H]⁺.

### Step 7: Synthesis of 1-(6-(2-hydroxyphenyl)pyridazin-4-yl)-4-(5-isopropylisoxazol-3-yl)piperidine-4-carbonitrile

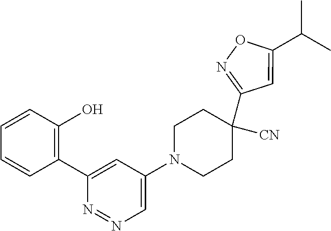 [SMILES: CC(C)c1cc(C2(C#N)CCN(c3cnnc(-c4ccccc4O)c3)CC2)no1]

To a solution of 1-(6-chloropyridazin-4-yl)-4-(5-isopropylisoxazol-3-yl)piperidine-4-carbonitrile (4.00 g, 12.1 mmol), (2-hydroxyphenyl)boronic acid (3.33 g, 24.1 mmol) in 1,4-dioxane (40 mL) and H₂O (4 mL) was added K₂CO₃ (5.00 g, 36.2 mmol) and RuPhos Pd G₃ (504 mg, 603 μmol) at 25° C., the mixture was degassed and purged with N₂ three times. The mixture was then heated to 90° C. and stirred until complete as judged by LCMS. Once complete, the residue was diluted with H₂O (50.0 mL) and extracted with EtOAc (50.0 mL×3). The combined organic layers were washed with brine (50.0 mL×3), dried over anhydrous Na₂SO₄, filtered, and concentrated under reduced pressure to give a crude residue. The residue was purified by flash column chromatography (petroleum ether in ethyl acetate—5 to 100%) to afford the desired product as a brown solid. (2.00 g, 42% yield). LCMS C₂₂H₂₃N₅O₂ requires 389.2, found m/z=390.3 [M+H]⁺.

### Step 8: Synthesis of Title Compound

To a solution of 1-(6-(2-hydroxyphenyl)pyridazin-4-yl)-4-(5-isopropylisoxazol-3-yl)piperidine-4-carbonitrile (2.00 g, 5.14 mmol) in THF (6 mL), MeOH (6 mL) and H₂O (6 mL) was added NaOH (2.05 g, 51.4 mmol) at 25° C., the mixture was heated to 80° C. and stirred until complete as judged by LCMS. Once complete, the reaction mixture was diluted with H₂O (20 mL) and extracted with EtOAc (20 mL×3). The combined organic layers were washed with brine (20 mL×2), dried over anhydrous Na₂SO₄, filtered, and concentrated under reduced pressure to give a crude residue. The crude residue was purified by RP-FC (26.0%-46.0%). The desired eluent was concentrated under reduced pressure to half volume, frozen at −78° C. and lyophilized to dryness to afford to desired compound as a yellow solid (1.14 g, 58% yield). LCMS C₂₂H₂₄N₄O₄ requires 408.2, found m/z=409.1 [M+H]⁺.

### Intermediate 11

### 1-(6-(2-hydroxyphenyl)pyridazin-4-yl)-4-(pyrazolo[1,5-a]pyridin-2-yl)piperidine-4-carboxylic acid

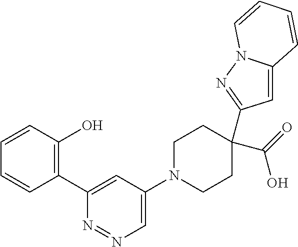 [SMILES: O=C(O)C1(c2cc3ccccn3n2)CCN(c2cnnc(-c3ccccc3O)c2)CC1]

### Step 1: Synthesis of ethyl 2-(pyrazolo[1,5-a]pyridin-2-yl)acetate

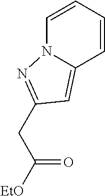 [SMILES: CCOC(=O)Cc1cc2ccccn2n1]

To a solution of 2-(pyrazolo[1,5-a]pyridin-2-yl)acetic acid (23.0 g, 130 mmol) in EtOH (115 mL) was added H₂SO₄ (6.40 g, 65.2 mmol, 3.48 mL) at 25° C., the reaction was then heated to 80° C. and allowed to stir until complete as judged by LCMS. Once complete, the reaction mixture was concentrated under vacuum to give a crude residue. The residue was quenched with ice-water (175 mL), followed by extraction with DCM (175 mL×3). The combined organic layers were dried over anhydrous Na₂SO₄, filtered, and concentrated under vacuum to afford the title compound as black-brown liquid (25.4 g). LCMS C₁₁H₁₂N₂O₂ requires 204.1, found m/z=205.1 [M+H]⁺.

### Step 2: Synthesis of 1-(tert-butyl) 4-ethyl 4-(pyrazolo[1,5-a]pyridin-2-yl)piperidine-1,4-dicarboxylate

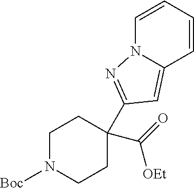 [SMILES: CCOC(=O)C1(c2cc3ccccn3n2)CCN(C(=O)OC(C)(C)C)CC1]

This intermediate was synthesized as described in Step 1 for Intermediate 9, using ethyl 2-(pyrazolo[1,5-a]pyridin-2-yl)acetate in place of methyl 2-(2-fluorophenyl)acetate (34.5 g). LCMS C₂₀H₂₇N₃O₄ requires 373.2, found m/z=318.2 [M−56+H]⁺.

### Step 3: ethyl 4-(pyrazolo[1,5-a]pyridin-2-yl)piperidine-4-carboxylate

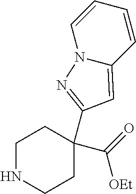 [SMILES: CCOC(=O)C1(c2cc3ccccn3n2)CCNCC1]

This intermediate was synthesized as described in Step 2 for Intermediate 9, using 1-(tert-butyl) 4-ethyl 4-(pyrazolo[1,5-a]pyridin-2-yl)piperidine-1,4-dicarboxylate (Step 13) in place of 1-(tert-butyl) 4-methyl 4-(2-fluorophenyl)piperidine-1,4-dicarboxylate (42.9 g). LCMS C₁₉H₂₀ClN₅O₂ requires 273.2, found m/z=274.2 [M+H]⁺.

### Step 4: Synthesis of ethyl 1-(6-chloropyridazin-4-yl)-4-(pyrazolo[1,5-a]pyridin-2-yl)piperidine-4-carboxylate

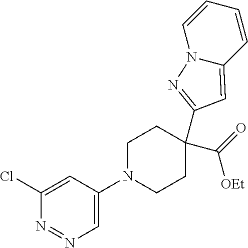 [SMILES: CCOC(=O)C1(c2cc3ccccn3n2)CCN(c2cnnc(Cl)c2)CC1]

This intermediate was synthesized as described for Intermediate 9 Step 3, using ethyl 4-(pyrazolo[1,5-a]pyridin-2-yl)piperidine-4-carboxylate in place of 4-(2-fluorophenyl)piperidine-4-carboxylate (11.0 g). LCMS C₁₉H₂₀ClN₅O₂ requires 385.1, found m/z=386.2 [M+H]⁺.

### Step 5: Synthesis of ethyl 1-(6-(2-hydroxyphenyl)pyridazin-4-yl)-4-(pyrazolo[1,5-a]pyridin-2-yl)piperidine-4-carboxylate

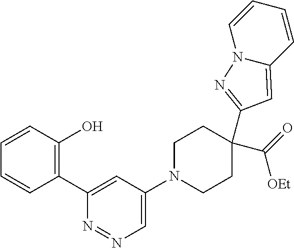 [SMILES: CCOC(=O)C1(c2cc3ccccn3n2)CCN(c2cnnc(-c3ccccc3O)c2)CC1]

This intermediate was prepared as described in Step 4 for Intermediate 9, using ethyl 1-(6-(2-hydroxyphenyl)pyridazin-4-yl)-4-(pyrazolo[1,5-a]pyridin-2-yl)piperidine-4-carboxylate in place of methyl 1-(6-chloropyridazin-4-yl)-4-(2-fluorophenyl)piperidine-4-carboxylate (42% yield over four steps). LCMS C₂₅H₂₅N₅O₃ requires 443.2, found m/z=444.2 [M+H]⁺.

Following basic hydrolysis, the title compound was obtained (2.50 g). LCMS C₂₃H₂₁N₅O₃ requires 415.2, found 416.2 [M+H]⁺.

### Intermediate 12

### 1-(6-(2-hydroxyphenyl)pyridazin-4-yl)-4-(1-methyl-1H-pyrazol-4-yl)piperidine-4-carboxylic acid

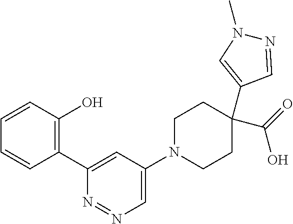 [SMILES: Cn1cc(C2(C(=O)O)CCN(c3cnnc(-c4ccccc4O)c3)CC2)cn1]

### Step 1: Synthesis of 1-(tert-butyl) 4-methyl 4-(1-methyl-1H-pyrazol-4-yl)piperidine-1,4-dicarboxylate

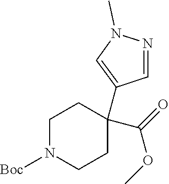 [SMILES: COC(=O)C1(c2cnn(C)c2)CCN(C(=O)OC(C)(C)C)CC1]

This intermediate was synthesized as described in Step 1 for Intermediate 9, using methyl 2-(1-methyl-1H-pyrazol-4-yl)acetate in place of methyl 2-(2-fluorophenyl)acetate (80.8 g). LCMS C₁₆H₂₅N₃O₄ requires 323.2, found m/z=268.2 [M−56+H]⁺.

### Step 2: Synthesis of methyl 4-(1-methyl-1H-pyrazol-4-yl)piperidine-4-carboxylate

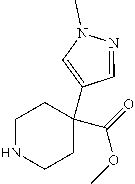 [SMILES: COC(=O)C1(c2cnn(C)c2)CCNCC1]

This intermediate was synthesized as described in Step 2 for Intermediate 9, using 1-(tert-butyl) 4-methyl 4-(1-methyl-1H-pyrazol-4-yl)piperidine-1,4-dicarboxylate in place of 1-(tert-butyl) 4-methyl 4-(2-fluorophenyl)piperidine-1,4-dicarboxylate (82.5 g). LCMS C₁₁H₁₇N₃O₂ requires 223.1, found m/z=224.3 [M+H]⁺.

### Step 3: Synthesis of methyl 1-(6-chloropyridazin-4-yl)-4-(1-methyl-1H-pyrazol-4-yl)piperidine-4-carboxylate

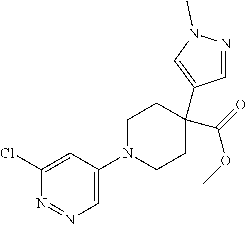 [SMILES: COC(=O)C1(c2cnn(C)c2)CCN(c2cnnc(Cl)c2)CC1]

This intermediate was synthesized as in Step 3 for Intermediate 9, using methyl 4-(1-methyl-1H-pyrazol-4-yl)piperidine-4-carboxylate in place of 4-(2-fluorophenyl)piperidine-4-carboxylate (7.8 g) LCMS C₁₅H₁₈ClN₅O₂ requires 335.1, found m/z=336.2 [M+H]⁺.

### Step 4: Synthesis of methyl 1-(6-(2-hydroxyphenyl)pyridazin-4-yl)-4-(1-methyl-1H-pyrazol-4-yl)piperidine-4-carboxylate

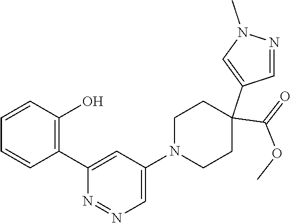 [SMILES: COC(=O)C1(c2cnn(C)c2)CCN(c2cnnc(-c3ccccc3O)c2)CC1]

This intermediate was synthesized as described in Step 4 for Intermediate 9, using methyl 1-(6-chloropyridazin-4-yl)-4-(1-methyl-1H-pyrazol-4-yl)piperidine-4-carboxylate in place of methyl 1-(6-chloropyridazin-4-yl)-4-(2-fluorophenyl)piperidine-4-carboxylate (4.1 g, 44% yield over four steps). LCMS C₂₁H₂₃N₅O₃ requires 393.2, found m/z=394.2 [M+H]⁺.

Following basic hydrolysis, the title compound was obtained (2.2 g, 79% yield). LCMS C₂₀H₂₁N₅O₃ requires 379.2, found 380.2 [M+H]⁺.

### Intermediate 13

### 1-(6-(2-hydroxyphenyl)pyridazin-4-yl)-4-(3-methylisoxazol-5-yl)piperidine-4-carboxylic acid

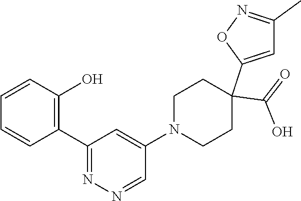 [SMILES: Cc1cc(C2(C(=O)O)CCN(c3cnnc(-c4ccccc4O)c3)CC2)on1]

### Step 1: Synthesis of 1-(tert-butyl) 4-methyl 4-(3-methylisoxazol-5-yl)piperidine-1,4-dicarboxylate

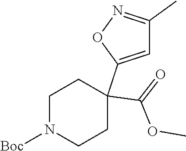 [SMILES: COC(=O)C1(c2cc(C)no2)CCN(C(=O)OC(C)(C)C)CC1]

This intermediate was prepared as described in Step 1 for Intermediate 9, using methyl 2-(3-methylisoxazol-5-yl)acetate in place of methyl 2-(2-fluorophenyl)acetate (57.8 g). LCMS C₁₆H₂₄N₂O₅ requires 324.2, found m/z=325.2 [M+H]⁺.

### Step 2: Synthesis of methyl 4-(3-methylisoxazol-5-yl)piperidine-4-carboxylate

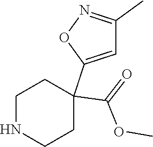 [SMILES: COC(=O)C1(c2cc(C)no2)CCNCC1]

This intermediate was synthesized as described in Step 2 for Intermediate 9, using 1-(tert-butyl) 4-methyl 4-(3-methylisoxazol-5-yl)piperidine-1,4-dicarboxylate in place of 1-(tert-butyl) 4-methyl 4-(2-fluorophenyl)piperidine-1,4-dicarboxylate (49.7 g). LCMS C₁₁H₁₆N₂O₃ requires 224.1, found m/z=225.1 [M+H]⁺.

### Step 3: Synthesis of methyl 1-(6-chloropyridazin-4-yl)-4-(3-methylisoxazol-5-yl)piperidine-4-carboxylate

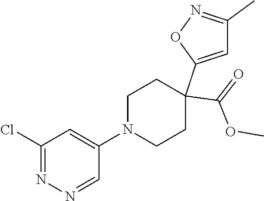 [SMILES: COC(=O)C1(c2cc(C)no2)CCN(c2cnnc(Cl)c2)CC1]

This intermediate was synthesized as described in Step 3 for Intermediate 9, using methyl 4-(3-methylisoxazol-5-yl)piperidine-4-carboxylate in place of methyl 4-(2-fluorophenyl)piperidine-4-carboxylate (5.1 g). LCMS C₁₅H₁₇ClN₄O₃ requires 336.1, found m/z=337.1 [M+H]⁺.

### Step 4: Synthesis of methyl 1-(6-(2-methoxyphenyl)pyridazin-4-yl)-4-(3-methylisoxazol-5-yl)piperidine-4-carboxylate

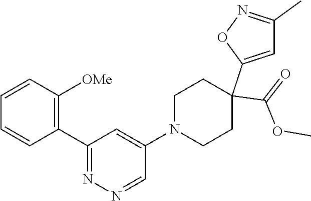 [SMILES: COC(=O)C1(c2cc(C)no2)CCN(c2cnnc(-c3ccccc3OC)c2)CC1]

This intermediate was synthesized as described in Step 7 for Intermediate 10, using methyl 1-(6-chloropyridazin-4-yl)-4-(3-methylisoxazol-5-yl)piperidine-4-carboxylate in place of 1-(6-chloropyridazin-4-yl)-4-(5-isopropylisoxazol-3-yl)piperidine-4-carbonitrile and (2-methoxyphenyl)boronic acid in place of (2-hydroxyphenyl)boronic acid (1.90 g, 36% yield over four steps). LCMS C₂₂H₂₄N₄O₄ requires 408.2, found m/z=409.1 [M+H]⁺.

### Step 5: Synthesis of methyl 1-(6-(2-hydroxyphenyl)pyridazin-4-yl)-4-(3-methylisoxazol-5-yl)piperidine-4-carboxylate

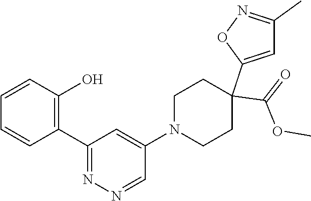 [SMILES: COC(=O)C1(c2cc(C)no2)CCN(c2cnnc(-c3ccccc3O)c2)CC1]

To a solution of methyl 1-(6-(2-methoxyphenyl)pyridazin-4-yl)-4-(3-methylisoxazol-5-yl)piperidine-4-carboxylate (1.60 g, 3.79 mmol) in DCM (50 mL) was added BBr₃ (2 M, 3.79 mL) at 0° C. The reaction was then warmed to 25° C. and allowed to stir until complete as judged by LCMS. Once complete, the reaction mixture was quenched by addition of HCl solution (1 N, 5.0 mL). The resulting mixture was extracted with EtOAc (10 mL×3), dried over anhydrous Na₂SO₄, filtered, and concentrated under reduced pressure to give a crude light-yellow solid which was taken forward without any further purification (3.80 g). LCMS C₂₁H₂₂N₄O₄ requires 394.2, found m/z=395.2 [M+H]⁺.

Following basic hydrolysis, the title compound was obtained (444 mgs, 31% yield). LCMS C₂₀H₂₀N₄04 requires 380.2, found m/z=381.1 [M+H]⁺.

### Intermediate 14

### 1-(6-(2-hydroxyphenyl)pyridazin-4-yl)-4-(5-methyl-1H-pyrazol-1-yl)piperidine-4-carboxylic acid

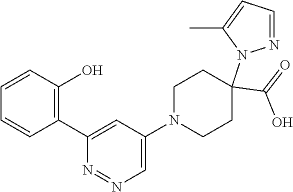 [SMILES: Cc1ccnn1C1(C(=O)O)CCN(c2cnnc(-c3ccccc3O)c2)CC1]

### Step 1: Synthesis of di-tert-butyl 1-(1-benzyl-4-(ethoxycarbonyl)piperidin-4-yl)hydrazine-1,2-dicarboxylate

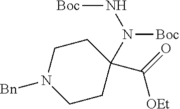 [SMILES: CCOC(=O)C1(N(NC(=O)OC(C)(C)C)C(=O)OC(C)(C)C)CCN(Cc2ccccc2)CC1]

To a solution of LDA (2 M in THF, 44.4 mL) in THF (120 mL) was added ethyl 1-benzylpiperidine-4-carboxylate (20.0 g, 80.8 mmol) dropwise at −70° C. and the mixture was stirred at −70° C. for 0.5 hr. Then to the mixture was added a solution of di-tert-butyl (E)-diazene-1,2-dicarboxylate (19.5 g, 84.9 mmol) in THF (40.0 mL) at −70° C. The mixture was warmed to 25° C. and allowed to stir until complete as judged by LCMS. Once complete, the reaction mixture was poured into saturated NH₄Cl solution (200 mL) and extracted with MTBE (100 mL×3). The organic layer was washed with brine (50.0 mL), dried over anhydrous Na₂SO₄, filtered, and concentrated under vacuum to afford a crude residue. The crude product was purified by flash column chromatography (petroleum ether in EtOAc—10% to 50%) to afford the title compound as a yellow solid (15.0 g, 39% yield). LCMS C₁₅H₂₁NO₂ requires 477.3, found m/z=478.4 [M+H]⁺.

### Step 2: Synthesis of ethyl 1-benzyl-4-hydrazineylpiperidine-4-carboxylate

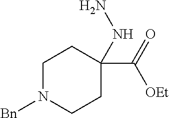 [SMILES: CCOC(=O)C1(NN)CCN(Cc2ccccc2)CC1]

This intermediate was synthesized as described in Step 2 for Intermediate 9, using di-tert-butyl 1-(1-benzyl-4-(ethoxycarbonyl)piperidin-4-yl)hydrazine-1,2-dicarboxylate in place of 1-(tert-butyl) 4-methyl 4-(2-fluorophenyl)piperidine-1,4-dicarboxylate (12.0 g, bis-HCl salt). LCMS C₁₅H₂₃N₃O₂ requires 277.2, found m/z=278.5 [M+H]⁺.

### Step 3: Synthesis of ethyl 1-benzyl-4-(5-methyl-1H-pyrazol-1-yl)piperidine-4-carboxylate

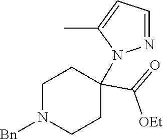 [SMILES: CCOC(=O)C1(n2nccc2C)CCN(Cc2ccccc2)CC1]

A solution of 4,4-dimethoxybutan-2-one (1.60 mL, 12.0 mmol) in AcOH (75.0 mL) was stirred at 120° C. for 1 hr. The mixture was cooled to 60° C. and to the mixture was added ethyl 1-benzyl-4-hydrazineylpiperidine-4-carboxylate (4.66 g, 12.04 mmol) at 60° C. The mixture was then heated and stirred at 120° C. until complete as judged by LCMS. Once complete, the reaction mixture adjusted pH to 10 with Na₂CO₃ solution, then extracted with EtOAc (60.0 mL×3). The combined organic layers were dried with anhydrous Na₂SO₄, filtered, and concentrated under vacuum to afford the crude product as a black/brown oil which was taken forward without any further purification (4.0 g). LCMS C₁₉H₂₅N₃O₂ requires 327.2, found m/z=328.4 [M=H]⁺.

### Step 4: Synthesis of ethyl 4-(5-methyl-1H-pyrazol-1-yl)piperidine-4-carboxylate

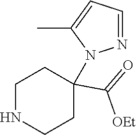 [SMILES: CCOC(=O)C1(n2nccc2C)CCNCC1]

To a solution of ethyl 1-benzyl-4-(5-methyl-1H-pyrazol-1-yl)piperidine-4-carboxylate (5.13 g, 15.6 mmol) in EtOH (40.0 mL) was added Pd/C (516 mg, 485 μmol, 10% purity) under N₂ atmosphere. The suspension was degassed and charged with H₂ three times. The mixture was charged with H₂ (50 psi) and allowed to stir at 25° C. until complete as judged by LCMS. Once complete, the reaction mixture was filtered through celite with EtOH washing, and the resulting filtrate was concentrated under vacuum to obtain a colorless oil which was taken forward with any further purification (3.4 g). LCMS C₁₂H₁₉N₃O₂ requires 237.2, found 238.5 [M+H]⁺.

### Step 5: Synthesis of ethyl 1-(6-chloropyridazin-4-yl)-4-(5-methyl-1H-pyrazol-1-yl)piperidine-4-carboxylate

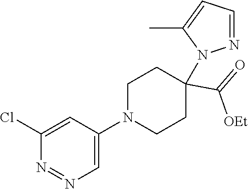 [SMILES: CCOC(=O)C1(n2nccc2C)CCN(c2cnnc(Cl)c2)CC1]

This intermediate was synthesized as in Step 3 for Intermediate 9, using ethyl 4-(5-methyl-1H-pyrazol-1-yl)piperidine-4-carboxylate in place of methyl 4-(2-fluorophenyl)piperidine-4-carboxylate (2.9 g). LCMS C₁₆H₂₀ClN₅O₂ requires 349.1, found m/z=350.3 [M+H]⁺.

### Step 6: Synthesis of ethyl 1-(6-(2-hydroxyphenyl)pyridazin-4-yl)-4-(5-methyl-1H-pyrazol-1-yl)piperidine-4-carboxylate

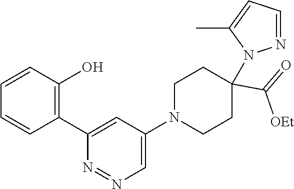 [SMILES: CCOC(=O)C1(n2nccc2C)CCN(c2cnnc(-c3ccccc3O)c2)CC1]

This intermediate was synthesized as described in Step 7 for Intermediate 10, using ethyl 1-(6-chloropyridazin-4-yl)-4-(5-methyl-1H-pyrazol-1-yl)piperidine-4-carboxylate in place of 1-(6-chloropyridazin-4-yl)-4-(5-isopropylisoxazol-3-yl)piperidine-4-carbonitrile (3.0 g). LCMS C₂₂H₂₅N₅O₃ requires 407.2, found m/z=408.3 [M+H]⁺.

Following Step 8 for Intermediate 10, the title compound was obtained (1.14 g, 10% yield over six steps). LCMS C₂₀H₂₁N₅O₃ requires 379.2, found 380.3 [M+H]⁺.

### Intermediate 15

### Synthesis of 2-(5-(4-(aminomethyl)-4-phenylpiperidin-1-yl)pyridazin-3-yl)phenol

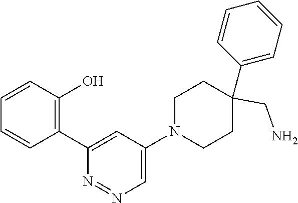 [SMILES: NCC1(c2ccccc2)CCN(c2cnnc(-c3ccccc3O)c2)CC1]

### Step 1: Synthesis of tert-butyl ((1-(6-chloropyridazin-4-yl)-4-phenylpiperidin-4-yl)methyl)carbamate

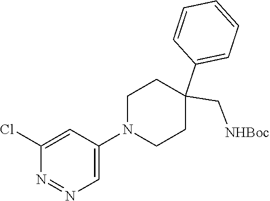 [SMILES: CC(C)(C)OC(=O)NCC1(c2ccccc2)CCN(c2cnnc(Cl)c2)CC1]

This intermediate was synthesized as described in Step 3 for Intermediate 9 using tert-butyl ((4-phenylpiperidin-4-yl)methyl)carbamate in place of methyl 4-(2-fluorophenyl)piperidine-4-carboxylate. LCMS C₂₁H₂₇ClN₄O₂ requires 402.2, found 403.2 [M+H]⁺.

### Step 2: Synthesis of tert-butyl ((1-(6-(2-hydroxyphenyl)pyridazin-4-yl)-4-phenylpiperidin-4-yl)methyl)carbamate

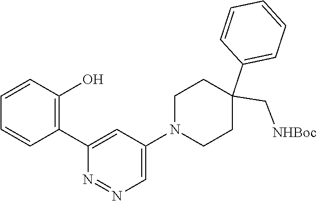 [SMILES: CC(C)(C)OC(=O)NCC1(c2ccccc2)CCN(c2cnnc(-c3ccccc3O)c2)CC1]

This intermediate was synthesized as described in Step 4 for Intermediate 9, using tert-butyl ((1-(6-chloropyridazin-4-yl)-4-phenylpiperidin-4-yl)methyl)carbamate in place of methyl 1-(6-chloropyridazin-4-yl)-4-(2-fluorophenyl)piperidine-4-carboxylate to afford the title compound as a yellow solid (1.13 g, 49% yield over two steps). LCMS C₂₇H₃₂N₄O₃ requires 460.3, found 461.3 [M+H]⁺.

Following basic hydrolysis, the title compound was obtained. LCMS C₂₂H₂₄N₄O requires 360.2, found m/z=361.2 [M+H]⁺.

### Intermediate 16

### Methyl 1-[6-(2-hydroxyphenyl)pyridazin-4-yl]-4-(2-methylphenoxy)piperidine-4-carboxylate

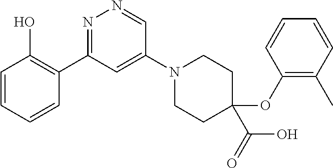 [SMILES: Cc1ccccc1OC1(C(=O)O)CCN(c2cnnc(-c3ccccc3O)c2)CC1]

Step 1: Synthesis of 1-[(tert-butoxy)carbonyl]-4-(2-methylphenoxy)piperidine-4-carboxylic acid

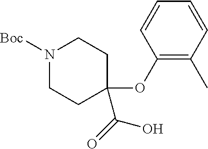 [SMILES: Cc1ccccc1OC1(C(=O)O)CCN(C(=O)OC(C)(C)C)CC1]

o-cresol (2.500 g, 23.11 mmol, 1.0 Equiv.) was dissolved in anhydrous THF (116 ml, 0.2 M). Powdered NaOH (4.624 g, 115.591 mmol, 5.0 eq) and 1-Boc-piperidin-4-one (13.819 g, 69.355 mmol, 3.0 Equiv.) were added to the solution at 0° C. Chloroform (9.26 ml, 115.591 mmol, 5.0 Equiv.) was added dropwise and the reaction mixture was stirred for 1 h at 0° C., followed by overnight stirring at RT. The reaction mixture was diluted with water and ethyl acetate. Layers were separated and aqueous layer was extracted twice with ethyl acetate. The aqueous layer was acidified to pH=2 (using 1 M HCl) and extracted three times with ethyl acetate. The organic phase extracted from the acidic aqueous phase was dried over MgSO₄, filtered and solvents were evaporated to give 1-[(tert-butoxy)carbonyl]-4-(2-methylphenoxy)piperidine-4-carboxylic acid (0.612 g, 1.825 mmol, 8%) as a yellow solid. ¹H NMR (DMSO-d₆): 12.86 (s, 1H), 7.18 (dd, J=7.6, 1.8 Hz, 1H), 7.08 (td, J=7.9, 1.8 Hz, 1H), 6.85 (td, J=7.4, 1.0 Hz, 1H), 6.59 (d, J=8.2 Hz, 1H), 3.77 (d, J=13.3 Hz, 2H), 3.15-2.86 (m, 2H), 2.23 (s, 3H), 2.11 (d, J=13.5 Hz, 2H), 2.01-1.74 (m, 2H), 1.42 (d, J=10.3 Hz, 9H).

### Step 2: Synthesis of methyl 4-(2-methylphenoxy)piperidine-4-carboxylate hydrochloride

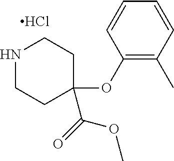 [SMILES: COC(=O)C1(Oc2ccccc2C)CCNCC1 ; Cl]

Thionyl chloride (0.293 ml, 4.014 mmol, 2.2 eq) was added carefully to a stirred solution of 1-[(tert-butoxy)carbonyl]-4-(2-methylphenoxy)piperidine-4-carboxylic acid (0.612 g, 1,825 mmol, 1 eq) in anhydrous methanol (6.08 ml, 0.3 M). The resulting solution was stirred at reflux overnight. Next, it was cooled down to RT and volatiles were evaporated to dryness. The residue was triturated with diethyl ether, filtered, washed with diethyl ether, collected and dried under reduced pressure to give methyl 4-(2-methylphenoxy)piperidine-4-carboxylate hydrochloride (0.494 g, 1.711 mmol, 94%) as a brown solid. ¹H NMR (DMSO-d₆): 9.01 (s, 2H), 7.23 (dd, J=7.6, 1.8 Hz, 1H), 7.11 (td, J=7.8, 1.8 Hz, 1H), 6.91 (td, J=7.4, 1.0 Hz, 1H), 6.48 (dd, J=8.2, 1.0 Hz, 1H), 3.75 (s, 3H), 3.23 (d, J=13.0 Hz, 2H), 2.95 (s, 2H), 2.30 (t, J=4.7 Hz, 4H), 2.27 (s, 3H).

### Step 3: Synthesis of methyl 1-(6-chloropyridazin-4-yl)-4-(2-methylphenoxy)piperidine-4-carboxylate

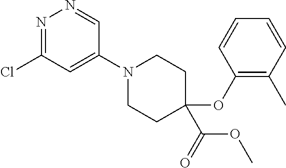 [SMILES: COC(=O)C1(Oc2ccccc2C)CCN(c2cnnc(Cl)c2)CC1]

Mixture of 3,5-Dichloropyridazine (0.255 g, 1.711 mmol, 1.0 eq), methyl 4-(2-methylphenoxy)piperidine-4-carboxylate hydrochloride (0.494 g, 1.711 mmol, 1.0 eq) in anhydrous NMP (3.42 ml, 0.5 M) and N,N-Diisopropylethylamine (0.894 ml, 5.134 mmol, 3.0 eq) was stirred under argon in closed vial at 100° C. for 1 h. Reaction was pouring it into cold water with vigorous stirring, The formed precipitate was filtered, washed with water, collected and dried under reduced pressure to give methyl 1-(6-chloropyridazin-4-yl)-4-(2-methylphenoxy)piperidine-4-carboxylate (0.47 g, 1.234 mmol, 72%) as a off-white solid. ¹H NMR (DMSO-d₆): 8.99 (d, J=2.7 Hz, 1H), 7.21 (d, J=7.3 Hz, 1H), 7.16-7.05 (m, 2H), 6.89 (t, J=7.4 Hz, 1H), 6.50 (d, J=8.1 Hz, 1H), 3.93 (d, J=13.6 Hz, 2H), 3.74 (s, 3H), 3.22 (td, J=13.4, 12.2, 3.4 Hz, 2H), 2.24 (s, 3H), 2.21-2.05 (m, 4H).

### Step 4: Synthesis of methyl 1-[6-(2-hydroxyphenyl)pyridazin-4-yl]-4-(2-methylphenoxy)piperidine-4-carboxylate

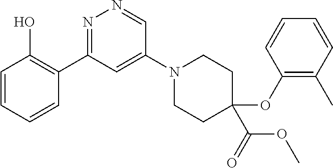 [SMILES: COC(=O)C1(Oc2ccccc2C)CCN(c2cnnc(-c3ccccc3O)c2)CC1]

methyl 1-(6-chloropyridazin-4-yl)-4-(2-methylphenoxy)piperidine-4-carboxylate (0.47 g, 1.234 mmol, 1.0 eq) was placed in a pressure vessel with K₂CO₃ (0.512 g, 3.70 mmol, 3.0 eq), 2-Hydroxyphenylboronic acid (0.255 g, 1.851 mmol, 1.5 eq) and Pd(PPh₃)₄ (0.143 g, 0.1234 mmol, 0.1 eq), followed by addition of dioxane (6.17 ml, 0.2 M) and water (1.23 ml, 1.0 M). Argon was bubbled through the mixture was 15 min. After that reaction mixture was stirring for about overnight at 100° C. After cooling down to RT, Celite was added and the mixture was evaporated under reduced pressure to give a dry-load for FC purification on silica gel (DCM-AcOEt 100:0 to 30:70) to give methyl 1-[6-(2-hydroxyphenyl)pyridazin-4-yl]-4-(2-methylphenoxy)piperidine-4-carboxylate (0.292 g, 0.696 mmol, 56%) as a off-white solid. ¹H NMR (DMSO-d₆): 14.54 (s, 1H), 8.98 (d, J=2.9 Hz, 1H), 8.10 (dd, J=8.4, 1.6 Hz, 1H), 7.59 (d, J=2.9 Hz, 1H), 7.34 (td, J=7.6, 1.5 Hz, 1H), 7.24-7.18 (m, 1H), 7.13 (t, J=7.7 Hz, 1H), 6.98-6.85 (m, 3H), 6.53 (d, J=8.2 Hz, 1H), 4.07 (t, J=15.0 Hz, 2H), 3.75 (s, 3H), 3.26 (s, 2H), 2.26 (s, 3H), 2.16 (dd, J=14.4, 4.2 Hz, 4H).

Following basic hydrolysis, the title compound was obtained (0.185 g, 0.443 mmol, 62%) as a white solid. ¹H NMR (DMSO-d₆): 14.00 (s, 2H), 8.98 (d, J=2.8 Hz, 1H), 8.11 (dd, J=8.4, 1.7 Hz, 1H), 7.59 (d, J=2.9 Hz, 1H), 7.34 (td, J=7.5, 1.6 Hz, 1H), 7.21 (d, 1H), 7.14 (td, J=7.8, 1.7 Hz, 1H), 6.93-6.83 (m, 3H), 6.66 (d, J=8.1 Hz, 1H), 4.10 (d, J=13.6 Hz, 2H), 3.33 (s, 2H), 2.20-2.08 (m, 7H).

### Intermediate 17

### 1-{6-[2-(methoxymethoxy)phenyl]pyridazin-4-yl}-4-phenylpiperidine-4-carboxylic acid

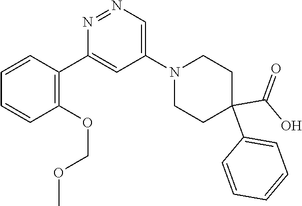 [SMILES: COCOc1ccccc1-c1cc(N2CCC(C(=O)O)(c3ccccc3)CC2)cnn1]

### Step 1: Synthesis of methyl 4-phenylpiperidine-4-carboxylate hydrochloride

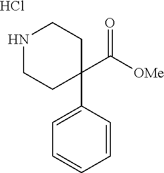 [SMILES: Cl ; COC(=O)C1(c2ccccc2)CCNCC1]

Thionyl chloride (5.402 ml, 74.466 mmol, 1.8 eq) was added carefully at 0 C to the stirred solution of 4-phenylpiperidine-4-carboxylic acid hydrochloride (10.0 g, 41.37 mmol, 1.0 eq) in anhydrous methanol (51.71 ml, 0.8 M). The resulting solution was stirred at reflux overnight. Next, it was cooled down to RT and volatiles were evaporated. The obtained solid was triturated with MTBE and filtered. The solid was collected and dried under reduced pressure which afforded methyl 4-phenylpiperidine-4-carboxylate hydrochloride (10.5 g, 39.004 mmol, 94%) as brown solid. LCMS (m/z): [M+H]⁺ calculated for C₁₃H₁₉NO₂+: 220.13. found: 220.65. ¹H NMR (300 MHz, DMSO-d₆) δ 9.22 (d, J=27.4 Hz, 2H), 7.46-7.28 (m, 5H), 3.64 (s, 3H), 3.23 (d, J=13.0 Hz, 2H), 2.92 (q, J=11.2 Hz, 2H), 2.55 (d, J=13.8 Hz, 2H), 2.19 (ddd, J=14.8, 11.5, 3.9 Hz, 2H).

### Step 2: Synthesis of methyl 1-(6-chloropyridazin-4-yl)-4-phenylpiperidine-4-carboxylate

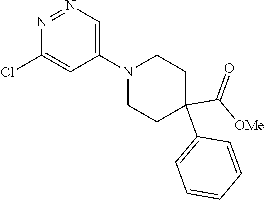 [SMILES: COC(=O)C1(c2ccccc2)CCN(c2cnnc(Cl)c2)CC1]

A 500 mL pressure vial equipped with a magnetic stirrer was charged with 3,5-dichloropyridazine (4.648 g, 31.203 mmol, 1.2 eq) and methyl 4-phenylpiperidine-4-carboxylate hydrochloride (7.0 g, 26.003 mmol, 1.0 eq). To this, dimethyl sulfoxide (52.01 ml, 0.5 M) and N,N-diisopropylethylamine (DIPEA) (27.176 ml, 156.018 mmol, 6.0 eq) were added. The reaction mixture was stirred at 100° C. overnight. DIPEA was evaporated and compound was precipitated in water followed by sonication, filtration, and drying which afforded methyl 1-(6-chloropyridazin-4-yl)-4-phenylpiperidine-4-carboxylate (8.5 g, 23.056 mmol, 89%) as brown solid. LCMS (m/z): [M+H]⁺ calculated for C₁₇H₁₉ClN₃O_{2+: 332.12}. found: 333.40. ¹H NMR (300 MHz, DMSO-d₆) δ 8.99 (d, J=2.7 Hz, 1H), 7.44-7.28 (m, 5H), 7.12 (d, J=2.7 Hz, 1H), 4.00 (dt, J=14.1, 4.0 Hz, 2H), 3.65 (s, 3H), 3.23-3.07 (m, 4H), 1.94 (ddd, J=13.6, 11.4, 4.0 Hz, 2H).

### Step 3: Synthesis of methyl 1-{6-[2-(methoxymethoxy)phenyl]pyridazin-4-yl}-4-phenylpiperidine-4-carboxylate

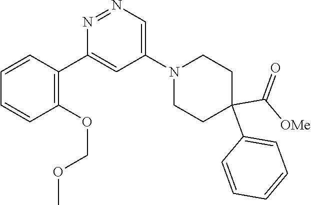 [SMILES: COCOc1ccccc1-c1cc(N2CCC(C(=O)OC)(c3ccccc3)CC2)cnn1]

A flame dried 500 mL pressure vial equipped with magnetic stirrer and argon balloon was charged with 2-(methoxymethoxy)phenylboronic acid (1.54 g, 8.463 mmol, 1.2 eq), and methyl 1-(6-chloropyridazin-4-yl)-4-phenylpiperidine-4-carboxylate (2.6 g, 7.052 mmol, 1.0 eq). To this dioxane (70.52 ml, 0.1 M) and potassium carbonate (2.924 g, 21.157 mmol, 3.0 eq) solution in water (14.1 ml, 0.5 M) were added. Argon was bubbled through the mixture for 20 min and then tetrakis(triphenylphosphine)palladium (0.815 g, 0.705 mmol, 0.1 eq) was added. The reaction vial was purged through argon before closing the cap and reaction mixture was stirred at 100° C. for 7 h. The compound was purified by FC using MTBE/DCM (0 to 70%) and methyl 1-{6-[2-(methoxymethoxy)phenyl]pyridazin-4-yl}-4-phenylpiperidine-4-carboxylate (2.56 g, 5.787 mmol, 82%) was obtained as yellow solid. LCMS (m/z): [M+H]⁺ calculated for C₂₅H₂₈ClN₃O₄+: 434.21, found: 434.90. ¹H NMR (300 MHz, DMSO-d₆) δ 8.98 (d, J=3.1 Hz, 1H), 7.61 (dd, J=7.6, 1.8 Hz, 1H), 7.48-7.33 (m, 5H), 7.33-7.18 (m, 3H), 7.12 (td, J=7.4, 1.1 Hz, 1H), 5.20 (s, 2H), 3.96 (d, J=13.7 Hz, 2H), 3.64 (s, 3H), 3.22-3.03 (m, 2H), 2.55 (s, 2H), 2.05-1.86 (m, 2H).

Following basic hydrolysis, the title compound was obtained (2.3 g, 5.318 mmol, 92%) as off-white solid. LCMS (m/z): [M+H]⁺ calculated for C₂₄H₂₆N₃O₄+: 420.19. found: 420.22. ¹H NMR (300 MHz, DMSO-d₆) δ 12.80 (s, 1H), 8.98 (d, J=3.1 Hz, 1H), 7.61 (dd, J=7.6, 1.8 Hz, 1H), 7.45-7.32 (m, 5H), 7.31-7.21 (m, 3H), 7.12 (td, J=7.4, 1.1 Hz, 1H), 5.20 (s, 2H), 3.97 (d, J=13.6 Hz, 2H), 3.57 (s, 5H), 3.15 (t, J=12.1 Hz, 2H), 1.97-1.82 (m, 2H).

### Intermediate 20

### 1-[6-(2-hydroxyphenyl)pyridazin-4-yl]-4-phenoxypiperidine-4-carboxylic acid

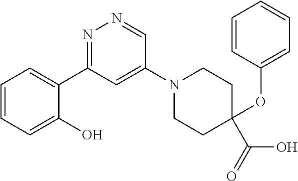 [SMILES: O=C(O)C1(Oc2ccccc2)CCN(c2cnnc(-c3ccccc3O)c2)CC1]

### Step 1: Synthesis of 1-[(tert-butoxy)carbonyl]-4-phenoxypiperidine-4-carboxylic acid

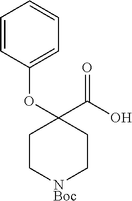 [SMILES: CC(C)(C)OC(=O)N1CCC(Oc2ccccc2)(C(=O)O)CC1]

Phenol (1.50 g, 15.938 mmol) was dissolved in anhydrous tetrahydrofuran (31.88 ml, 0.5 M) and cooled to 0° C. Powdered NaOH (3.19 g, 79.69 mmol) and 1-Boc-piperidin-4-one (9.53 g, 47.82 mmol) were added followed by addition of chloroform (6.38 ml, 79.69 mmol) dropwise and the resulting mixture was stirred for 1 h at 0° C., followed by overnight stirring at room temperature. Once complete, the reaction mixture was diluted with water and extracted twice with ethyl acetate. The aqueous layer was acidified to pH=2 (using 0.5 M HCl) and extracted with ethyl acetate (×2). The combined organic extracts from the acidic wash were dried over magnesium sulfate, filtered, and evaporated. The crude product was obtained as a yellow oil (2.11 g, 37%). LCMS: C₁₇H₂₃NO₅ requires 321.3. found: m/z=322.1 [M+H]⁺.

¹H NMR (300 MHz, DMSO-d₆) δ 12.99 (s, 1H), 7.32-7.25 (m, 2H), 7.02-6.94 (m, 1H), 6.90-6.84 (m, 2H), 3.75-3.65 (m, 2H), 3.14-2.99 (m, 2H), 2.04 (d, J=13.9 Hz, 2H), 1.93-1.86 (m, 2H), 1.39 (s, 9H).

### Step 2: Synthesis of 4-phenoxypiperidine-4-carboxylic acid hydrochloride

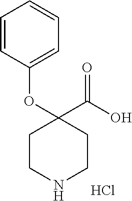 [SMILES: O=C(O)C1(Oc2ccccc2)CCNCC1 ; Cl]

1-[(tert-butoxy)carbonyl]-4-phenoxypiperidine-4-carboxylic acid (2.10 g, 5.89 mmol) was dissolved in anhydrous dichloromethane (58.95 mL, 0.1 M) and a solution of 4 M HCl in dioxane (73.69 mL, 294.7 mmol) was added and the resulting solution was stirred at room temperature for 72 h. Once complete, the solvent was removed under reduce pressure, to afford 4-phenoxypiperidine-4-carboxylic acid hydrochloride (1.59 g, 92%) as a tan solid. LCMS: C₁₂H₁₅NO₃ requires 221.7, found: m/z=222.1 [M+H]+.

NMR (300 MHz, DMSO-d6) δ 13.65 (s, 1H), 9.13 (d, J=21.6 Hz, 2H), 7.36-7.27 (m, 2H), 7.05-6.98 (m, 1H), 6.93-6.86 (m, 2H), 3.19 (d, J=12.4 Hz, 2H), 3.06-2.93 (m, 2H), 2.27-2.18 (m, 4H).

### Step 3: Synthesis of 1-(6-chloropyridazin-4-yl)-4-phenoxypiperidine-4-carboxylic acid

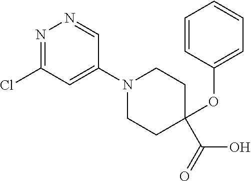 [SMILES: O=C(O)C1(Oc2ccccc2)CCN(c2cnnc(Cl)c2)CC1]

To a solution of 3,5-dichloropyridazine (1.72 g, 11.59 mmol) in anhydrous dimethylformamide (23.19 ml, 0.2 M) was added 4-phenoxypiperidine-4-carboxylic acid hydrochloride (1.44 g, 4.638 mmol) and DIPEA (4.85 ml, 23.83 mmol). The resulting mixture was stirred at 100° C. for 3 h. Once complete, the reaction was quenched citric acid to pH=−5 and extracted with dichloromethane 3×. The organic phase was dried over magnesium sulfate, filtered, and concentrated in vacuo. The residue was purified by flash-chromatography (DCM/MeOH, 100:0 to 90:10) to afford 1-(6-chloropyridazin-4-yl)-4-phenoxypiperidine-4-carboxylic acid (824 mg, 48%) as a tan powder. LCMS: C₁₆H₁₆ClN₃O₃ requires 333.7. found: m/z=333.8 [M+H]⁺.

1H NMR (300 MHz, DMSO-d6) δ 13.43 (s, 1H), 8.97 (d, J=2.7 Hz, 1H), 7.30 (tt, J=7.4, 2.4 Hz, 2H), 7.11 (d, J=2.7 Hz, 1H), 7.04-6.97 (m, 1H), 6.93-6.86 (m, 2H), 3.86 (d, J=13.9 Hz, 2H), 3.32-3.24 (m, 2H), 2.16-2.04 (m, 4H).

### Step 4: Synthesis of 1-{6-[2-(methoxymethoxy)phenyl]pyridazin-4-yl}-4 phenoxypiperidine-4-carboxylic acid

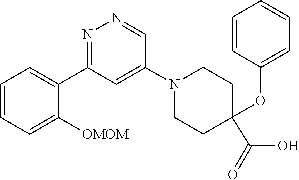

2-(Methoxymethoxy)phenylboronic acid (0.565 g, 3.11 mmol), 1-(6-chloropyridazin-4-yl)-4-phenoxypiperidine-4-carboxylic acid (0.768 g, 2.07 mmol), anhydrous K₂CO₃ (0.859 g, 6.21 mmol) and tetrakis(triphenylphosphine)palladium (0.239 g, 0.207 mmol) were added to a sealed vessel and under argon atmosphere. Dioxane (20.71 mL) and H₂O (4.14 mL) were added sequentially, and the resulting solution was degassed with an argon balloon for 5 minutes. The mixture was stirred overnight at 100° C. Once complete, the solvent was removed under reduced pressure and the resulting residue was purified by flash-chromatography (DCM/MeOH, 98:02 to 85:15) to afford 1-{6-[2-(methoxymethoxy)phenyl]pyridazin-4-yl}-4 phenoxypiperidine-4-carboxylic acid (503 mg, 54%). LCMS: C₂₄H₂₅N₃O₅ requires 435.4. found: m/z=436.5 [M+H]⁺.

¹H NMR (300 MHz, DMSO-d₆) δ 13.68 (s, 1H), 8.97 (d, J=3.0 Hz, 1H), 7.62 (dd, J=7.6, 1.8 Hz, 1H), 7.41 (ddd, J=9.0, 7.3, 1.8 Hz, 1H), 7.33-7.21 (m, 4H), 7.12 (td, J=7.4, 1.1 Hz, 1H), 6.98 (t, J=7.4 Hz, 1H), 6.91 (d, J=7.9 Hz, 2H), 5.20 (s, 2H), 3.84 (d, J=13.4 Hz, 2H), 3.31 (s, 5H), 2.16-2.02 (m, 4H).

### Step 5: Synthesis of Title Compound

1-{6-[2-(methoxymethoxy)phenyl]pyridazin-4-yl}-4-phenoxypiperidine-4-carboxylic acid (0.346 g, 0.763 mmol) was dissolved in anhydrous dichloromethane (7.63 mL, 0.1 M) and trifluoroacetic acid (2.92 mL, 38.14 mmol) was added dropwise. The resulting solution was stirred at room temperature overnight. Once complete, the solvent was removed under reduced pressure and the residue was purified via RP-FC to afford the title compound (120 mg, 29%) as a trifluoroacetate salt.

¹H NMR (300 MHz, DMSO-d₆) δ 13.63 (s, 2H), 8.98 (d, J=2.9 Hz, 1H), 7.86 (d, J=7.9 Hz, 1H), 7.56 (d, J=3.0 Hz, 1H), 7.42-7.29 (m, 3H), 7.05-6.90 (m, 5H), 4.11 (d, J=13.6 Hz, 2H), 3.52-3.41 (m, 2H), 2.23-2.12 (m, 4H).

### Intermediate 21

### 1-[6-(2-hydroxyphenyl)pyridazin-4-yl]-4-[(1-methyl-1H-pyrazol-3-yl)oxy]piperidine-4-carboxylic acid

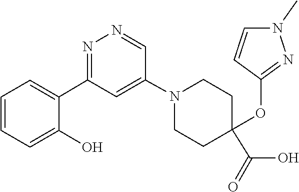 [SMILES: Cn1ccc(OC2(C(=O)O)CCN(c3cnnc(-c4ccccc4O)c3)CC2)n1]

### Step 1: Synthesis of 1-[(tert-butoxy)carbonyl]-4-[(1-methyl-1H-pyrazol-3-yl)oxy]piperidine-4-carboxylic acid

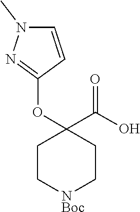 [SMILES: Cn1ccc(OC2(C(=O)O)CCN(C(=O)OC(C)(C)C)CC2)n1]

A solution of 2-methyl-1H-pyrazol-5-one (3.0 g, 30.58 mmol) in anhydrous tetrahydrofuran (152.9 ml, 0.2 M) was cooled to 0° C. and powdered NaOH (6.116 g, 152.89 mmol) and 1-Boc-piperidin-4-one (18.279 g, 91.738 mmol, 3.0 eq) were added sequentially. Chloroform anhydrous (12.249 ml, 152.897 mmol, 5.0 eq) was added dropwise to the solution and the resulting mixture was stirred overnight at room temperature. Once complete, the reaction mixture was diluted with water and extracted twice with ethyl acetate. The aqueous layer was acidified to pH=2 (using 0.5 M HCl) and extracted with ethyl acetate (×2). The combined organic extracts from the acidic wash were dried over magnesium sulfate, filtered, and evaporated. The crude product was obtained as a yellow oil (3.60 g, 36%). LCMS: C₁₅H₂₃N₃O₅ requires 325.1. found: m/z=326.4 [M+H]⁺.

1H NMR (300 MHz, DMSO-d6) δ 12.83 (s, 1H), 7.45 (d, J=2.3 Hz, 1H), 5.62 (d, J=2.3 Hz, 1H), 3.73 (dt, J=13.6, 3.7 Hz, 2H), 3.63 (s, 3H), 3.05 (s, 2H), 2.13 (d, J=13.9 Hz, 2H), 1.83 (ddd, J=14.0, 11.6, 4.7 Hz, 2H), 1.41 (s, 9H).

### Step 2: Synthesis of methyl 4-[(1-methyl-1H-pyrazol-3-yl)oxy]piperidine-4-carboxylate hydrochloride

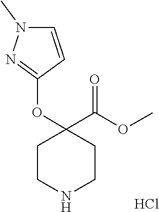

A solution of 1-[(tert-butoxy)carbonyl]-4-[(1-methyl-1H-pyrazol-3-yl)oxy]piperidine-4-carboxylic acid (3.6 g, 11.06 mmol) in MeOH (110.64 ml, 0.1 M) was cooled to 0° C. and SOCl₂ (2.40 mL, 33.19 mmol) was added dropwise. The resulting solution was stirred at reflux overnight. Once complete, the reaction was cooled to room temperature and volatiles were evaporated. The obtained solid was triturated with MTBE and filtered. The solid was collected and dried under reduced pressure to afford methyl 4-[(1-methyl-1H-pyrazol-3-yl)oxy]piperidine-4-carboxylate hydrochloride (2.88 g, 94%). LCMS: C₁₁H₁₇N₃O₃ requires 239.1. found: m/z=240.2 [M+H]⁺.

1H NMR (300 MHz, DMSO-d6) δ 9.53 (s, 2H), 7.49 (d, J=2.3 Hz, 1H), 5.65 (d, J=2.3 Hz, 1H), 3.66 (s, 3H), 3.62 (s, 3H), 3.23-3.14 (m, 2H), 3.08-2.89 (m, 2H), 2.35-2.15 (m, 4H).

### Step 3: Synthesis of methyl 1-(6-chloropyridazin-4-yl)-4-[(1-methyl-1H-pyrazol-3-yl)oxy]piperidine-4-carboxylate

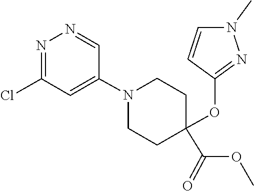 [SMILES: COC(=O)C1(Oc2ccn(C)n2)CCN(c2cnnc(Cl)c2)CC1]

To a stirred solution of methyl 4-[(1-methyl-1H-pyrazol-3-yl)oxy]piperidine-4-carboxylate hydrochloride (2.88 g, 10.44 mmol) in DMSO (52.23 ml, 0.2 M) was added N,N-diisopropylethylamine (10.9 ml, 62.67 mmol), followed by 3,5-Dichloropyridazine (1.71 g, 11.49 mmol). The reaction mixture was stirred at 100° C. overnight. Once complete, the excess of DIPEA was removed under reduced pressure. Cold water was added to the solution and mixture was sonicated for 15 minutes. The resulting solid was filtered to afford methyl 1-(6-chloropyridazin-4-yl)-4-[(1-methyl-1H-pyrazol-3-yl)oxy]piperidine-4-carboxylate (3.63 g, 10.319 mmol). LCMS: C₁₅H₁₈ClN₅O₃ requires 351.1. found: m/z=352.3 [M+H]⁺.

1H NMR (300 MHz, DMSO-d6) δ 8.98 (d, J=2.7 Hz, 1H), 7.48 (d, J=2.3 Hz, 1H), 7.12 (d, J=2.7 Hz, 1H), 5.64 (d, J=2.3 Hz, 1H), 3.91 (d, J=13.5 Hz, 2H), 3.65 (s, 3H), 3.64 (s, 3H), 3.26 (d, J=11.1 Hz, 2H), 2.22 (d, J=14.1 Hz, 2H), 2.10-1.94 (m, 2H).

### Step 4: Synthesis of methyl 1-[6-(2-hydroxyphenyl)pyridazin-4-yl]-4-[(1-methyl-1H-pyrazol-3-yl)oxy]piperidine-4-carboxylate

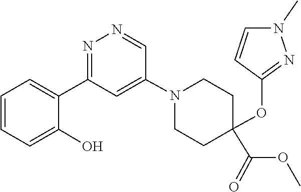 [SMILES: COC(=O)C1(Oc2ccn(C)n2)CCN(c2cnnc(-c3ccccc3O)c2)CC1]

Methyl 1-(6-chloropyridazin-4-yl)-4-[(1-methyl-1H-pyrazol-3-yl)oxy]piperidine-4-carboxylate (2.0 g, 5.685 mmol, 1.0 eq), K₂CO₃ (2.357 g, 17.056 mmol, 3.0 eq) and 2-Hydroxyphenylboronic acid (0.863 g, 6.254 mmol, 1.1 eq) were added to a sealed vessel under argon atmosphere. Dioxane (28.43 ml, 0.2 M) and Water (5.69 ml, 1.0 M) were sequentially added and the resulting solution was degassed with an argon balloon for 15 mins. Tetrakis(triphenylphosphine)palladium (0.657 g, 0.569 mmol, 0.1 eq) was then added to the mixture and resulting solution was stirred at 100° C. for 16 h. Once complete, the solvents were evaporated and the crude mixture was purified by flash chromatography (DCM/MeOH, 98:02 to 85:15) to afford methyl 1-[6-(2-hydroxyphenyl)pyridazin-4-yl]-4-[(1-methyl-1H-pyrazol-3-yl)oxy]piperidine-4-carboxylate (1.1 g, 42%) as yellow solid. LCMS: C₂₁H₂₃N₅O₄ requires 409.4. found: m/z=410.4 [M+H]+.

1H NMR (300 MHz, DMSO-d6) 1H NMR δ 14.57 (s, 1H), 8.98 (d, J=2.8 Hz, 1H), 8.12 (dd, J=8.4, 1.7 Hz, 1H), 7.70-7.53 (m, 4H), 7.50 (d, J=2.3 Hz, 1H), 7.34 (ddd, J=8.4, 7.3, 1.6 Hz, 1H), 6.98-6.87 (m, 2H), 5.67 (d, J=2.3 Hz, 1H), 4.16-3.97 (m, 2H), 3.67 (s, 3H), 3.45-3.29 (m, 3H), 2.28 (d, J=14.0 Hz, 2H), 2.08 (ddd, J=14.4, 11.2, 4.3 Hz, 2H).

Following basic hydrolysis, the title compound was obtained as an off-white solid (0.116 g, 12%). LCMS: C₂₀H₂₁N₅O₄ requires 395.4. found: m/z=396.2 [M+H]+.

1H NMR (300 MHz, DMSO-d6) δ 13.76 (s, 1H), 8.98 (d, J=2.8 Hz, 1H), 8.12 (dd, J=8.4, 1.7 Hz, 1H), 7.59 (d, J=2.9 Hz, 1H), 7.49 (d, J=2.3 Hz, 1H), 7.34 (td, J=7.6, 1.6 Hz, 1H), 6.99-6.87 (m, 2H), 5.67 (d, J=2.3 Hz, 1H), 4.14-4.03 (m, 2H), 3.66 (s, 3H), 3.43-3.34 (m, 2H), 2.34-2.23 (m, 2H), 2.07 (ddd, J=15.1, 11.4, 4.3 Hz, 2H).

### Intermediate 22

### 4-cyclopropoxy-1-[6-(2-hydroxyphenyl)pyridazin-4-yl]piperidine-4-carboxylic acid

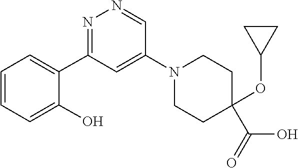 [SMILES: O=C(O)C1(OC2CC2)CCN(c2cnnc(-c3ccccc3O)c2)CC1]

### Step 1: Synthesis of benzyl 4-cyclopropoxy-4-(hydroxymethyl)piperidine-1-carboxylate

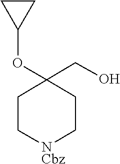 [SMILES: O=C(OCc1ccccc1)N1CCC(CO)(OC2CC2)CC1]

A flask was charged with benzyl 1-oxa-6-azaspiro[2.5]octane-6-carboxylate (5.41 g, 21.86 mmol), and cyclopropanol (28.14 g, 484 mmol) under argon atmosphere. The mixture was cooled down to 15° C. using an ice water bath. Boron trifluoride diethyl etherate (6.887 g, 48.53 mmol) was then added dropwise while maintaining the temperature of the bath at 15° C. After the addition was complete, the cooling bath was removed and stirring was continued for 1 h. DCM was added, followed by slow addition of water. The contents were transferred to a separatory funnel and the layers were separated. The aqueous layer was washed with DCM 3× and the combined organic layers were dried over Na₂SO₄, filtered and concentrated under reduced pressure. The resulting residue was purified via RP-FC column to afford benzyl 4-cyclopropoxy-4-(hydroxymethyl)piperidine-1-carboxylate (3.07, 46%) as a colorless oil. LCMS: C₁₇H₂₃NO₄ requires 305.3. found: m/z=306.6 [M+H]+.

¹H NMR (300 MHz, DMSO-d₆) 7.42-7.29 (m, 5H), 5.07 (s, 2H), 4.63 (t, J=5.5 Hz, 1H), 3.71 (dt, J=13.2, 4.2 Hz, 2H), 3.48 (d, J=5.5 Hz, 2H), 3.27 (tt, J=6.2, 3.1 Hz, 1H), 3.09 (s, 2H), 1.75-1.63 (m, 2H), 1.47 (ddd, J=13.8, 11.4, 4.6 Hz, 2H), 0.52-0.36 (m, 4H).

### Step 2: Synthesis of benzyl 4-cyclopropoxy-4-formylpiperidine-1-carboxylate

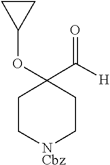 [SMILES: O=CC1(OC2CC2)CCN(C(=O)OCc2ccccc2)CC1]

Dess-Martin periodinane, 95% (5.543 g, 13.069 mmol) was added to a stirring solution of benzyl 4-cyclopropoxy-4-(hydroxymethyl)piperidine-1-carboxylate (3.07 g, 10.05 mmol) in dichloromethane (50.27 ml, 0.2 M) and the resulting mixture was stirred at room temperature for 2 h.

Once complete, DCM (50 mL) and water (50 M) were added, followed by sat. aq. Na₂S₂O₃ (50 ml) and the mixture was stirred for 15 min. The contents were transferred to a separatory funnel and the layers were separated. The aqueous layer was washed with DCM 2× and the combined organic layers were washed with brine, collected, dried over Na₂SO₄, filtered and concentrated in vacuo to afford benzyl 4-cyclopropoxy-4-formylpiperidine-1-carboxylate (2.73 g, 81%) as a colorless oil. LCMS: C₁₇H₂₁NO₄ requires 303.3. found: m/z=304.2 [M+H]⁺.

1H NMR (300 MHz, DMSO-d6) 9.58 (s, 1H), 7.42-7.32 (m, 5H), 5.08 (s, 2H), 3.69 (dt, J=13.5, 4.6 Hz, 2H), 3.32-3.14 (m, 3H), 1.87-1.76 (m, 2H), 1.67 (ddd, J=14.3, 10.4, 4.5 Hz, 2H), 0.58-0.44 (m, 4H).

### Step 3: Synthesis of 1-[(benzyloxy)carbonyl]-4-cyclopropoxypiperidine-4-carboxylic acid

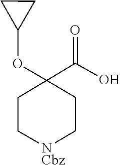 [SMILES: O=C(OCc1ccccc1)N1CCC(OC2CC2)(C(=O)O)CC1]

A flask was charged with benzyl 4-cyclopropoxy-4-formylpiperidine-1-carboxylate (2.73 g, 8.09 mmol) and potassium phosphate monobasic (3.30 g, 24.29 mmol). T-Butanol (80.99 ml), Water (20.25 ml) and 2-Methyl-2-butene (10.297 ml, 97.19 mmol) were then added sequentially. Sodium chlorite (2.75 g, 24.29 mmol) was added and the reaction mixture was stirred at room temperature for 2 h. Once complete by LCMS, the reaction was diluted with DCM and the pH was adjusted to ˜12 using 1M NaOH. The contents were transferred to a separatory funnel and the layers were separated. The aqueous layer was then acidified with 1 M HCl to pH˜2 and extracted with EtOAc 3×. The combined organic layers were dried over Na₂SO₄, filtered and evaporated to afford 1-[(benzyloxy)carbonyl]-4-cyclopropoxypiperidine-4-carboxylic acid (1.76 g, 65% yield) as a colorless oil. LCMS: C₁₇H₂₁NO₅ requires 318.3. found: m/z=319.3 [M+H]+.

¹H NMR (300 MHz, DMSO-d₆) 12.80 (s, 1H), 7.41-7.29 (m, 5H), 5.08 (s, 2H), 3.60 (dt, J=13.4, 4.6 Hz, 2H), 3.30-3.16 (m, 3H), 1.91-1.79 (m, 4H), 0.59-0.37 (m, 4H).

### Step 4: 1-benzyl 4-methyl 4-cyclopropoxypiperidine-1,4-dicarboxylate

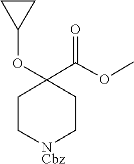 [SMILES: COC(=O)C1(OC2CC2)CCN(C(=O)OCc2ccccc2)CC1]

(Trimethylsilyl)diazomethane, (2M in hexanes) (17.02 ml, 34.03 mmol) was added dropwise to a cooled (10° C.) solution of 1-[(benzyloxy)carbonyl]-4-cyclopropoxypiperidine-4-carboxylic acid (1.76 g, 5.24 mmol) in methanol (20.94 ml, 0.25 M). Addition was continued until the light yellow color persisted in the solution. The cooling bath was then removed and the reaction was stirred at room temperature for 1 h. Once complete, acetic acid was added dropwise until the yellow color disappeared and the solution became colorless. Residual solvents were evaporated and the residue was purified by chromatography (hexane/MTBE, 100:0 to 75:25) to afford 1-benzyl 4-methyl 4-cyclopropoxypiperidine-1,4-dicarboxylate (1.39 g, 79%) as a colorless oil. LCMS: C₁₈H₂₃NO₅ requires 333.3. found: m/z=334.2 [M+H]+.

¹H NMR (300 MHz, DMSO-d₆) 7.42-7.29 (m, 5H), 5.08 (s, 2H), 3.70 (s, 3H), 3.64-3.53 (m, 2H), 3.30-3.19 (m, 3H), 1.96-1.79 (m, 4H), 0.52-0.38 (m, 4H).

### Step 5: Synthesis of methyl 4-cyclopropoxypiperidine-4-carboxylate

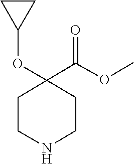

1-benzyl-4-methyl-4-cyclopropoxypiperidine-1,4-dicarboxylate (1.29 g, 3.90 mmol) was dissolved in THF (37 ml, 0.02 M) in a 250 ml three-neck-RBF equipped with argon balloon and hydrogen balloon. The reaction mixture was degassed with argon and Pd/C (10% by weight; 0.62 g, 5.8 mmol) was added followed again by degassing with argon and filling with hydrogen. The reaction was stirred at room temperature overnight. The reaction mixture was filtered through a celite pad and evaporated to dryness to provide methyl 4-cyclopropoxypiperidine-4-carboxylate (0.78 g, 96%) as a gray solid.

¹H NMR (300 MHz, DMSO-d₆) δ 3.68 (s, 3H), 3.22-3.17 (m, 1H), 2.81-2.68 (m, 2H), 2.63-2.53 (m, 2H), 1.92-1.81 (m, 2H), 1.76-1.62 (m, 2H), 0.51-0.33 (m, 4H).

### Step 6: Synthesis of methyl 1-(6-chloropyridazin-4-yl)-4-cyclopropoxypiperidine-4-carboxylate

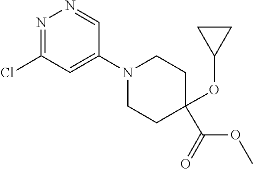 [SMILES: COC(=O)C1(OC2CC2)CCN(c2cnnc(Cl)c2)CC1]

A mixture of 3,5-Dichloropyridazine (0.55 g, 3.72 mmol), methyl 4-cyclopropoxypiperidine-4-carboxylate (0.78 g, 3.72 mmol) in N-methyl-2-pyrrolidone (7.44 ml, 0.5 M) and N,N-diisopropylethylamine (1.9 ml, 11.16 mmol) was stirred under argon atmosphere in closed vial at 100° C. for 1 h. Once complete, the reaction was quenched with cold water and extracted with ethyl acetate 3×. The combined organic extracts were dried over Na₂SO₄, filtered and concentrated in vacuo to afford methyl 1-(6-chloropyridazin-4-yl)-4-cyclopropoxypiperidine-4-carboxylate (1.4 g, 87%) as a sticky brown solid that was used in next step without additional purification. LCMS: C₁₄H₁₈ClN₃O₃ requires 311.7. found: m/z=313.0 [M+H]⁺.

¹H NMR (300 MHz, DMSO-d₆) δ 8.97 (d, J=2.7 Hz, 1H), 7.11 (d, J=2.7 Hz, 1H), 3.71 (s, 3H), 3.45-3.36 (m, 1H), 2.22-2.14 (m, 2H), 1.99-1.86 (m, 6H), 0.53-0.38 (m, 4H).

### Step 7: Synthesis of methyl 4-cyclopropoxy-1-[6-(2-hydroxyphenyl)pyridazin-4-yl]piperidine-4-carboxylate

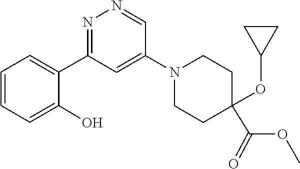 [SMILES: COC(=O)C1(OC2CC2)CCN(c2cnnc(-c3ccccc3O)c2)CC1]

Methyl-1-(6-chloropyridazin-4-yl)-4-cyclopropoxypiperidine-4-carboxylate (1.4 g, 3.59 mmol) was added to a pressure vessel with K₂CO₃ (1.49 g, 10.78 mmol) and 2-Hydroxyphenylboronic acid (0.74 g, 5.39 mmol). Dioxane (18.0 ml, 0.2 M) and H₂O (3.6 ml, 1.0 M) were added and the resulting mixture was degassed with an argon balloon for 15 mins. Tetrakis(triphenylphosphine)palladium (0.415 g, 0.359 mmol, 0.1 eq) was added to the reaction mixture and the solution was stirred at 100° C. for 18 h. Once complete, the reaction was concentrated in vacuo and the resulting residue was purfied by chromatography (Hexane/EtOAc, 0-100%) to provide methyl 4-cyclopropoxy-1-[6-(2-hydroxyphenyl)pyridazin-4-yl]piperidine-4-carboxylate (0.58 g, 44% yield) as a yellow solid. LCMS: C₂₀H₂₃N₃O₄ requires 369.4. found: m/z=370.7 [M+H]+.

¹H NMR (300 MHz, DMSO-d6) δ 14.58 (s, 1H), 8.97 (d, J=2.8 Hz, 1H), 8.11 (dd, J=8.4, 1.7 Hz, 1H), 7.58 (d, J=2.9 Hz, 1H), 7.43-7.18 (m, 1H), 7.01-6.84 (m, 2H), 3.91-3.78 (m, 2H), 3.72 (s, 3H), 3.56-3.43 (m, 2H), 3.31-3.26 (m, 1H), 2.02 (t, J=5.6 Hz, 4H), 0.57-0.41 (m, 4H).

Following basic hydrolysis, the title compound was obtained (468.4 mg, 82%) as a off-white solid. LCMS: C₁₉H₂₁N₃O₄ requires 355.3. found: m/z=356.3 [M+H]⁺.

¹H NMR (300 MHz, DMSO-d6) δ 13.57 (s, 2H), 8.96 (d, J=2.8 Hz, 1H), 8.11 (dd, J=8.4, 1.7 Hz, 1H), 7.57 (d, J=2.9 Hz, 1H), 7.34 (ddd, J=8.5, 7.2, 1.6 Hz, 1H), 6.99-6.84 (m, 2H), 3.90-3.77 (m, 2H), 3.54-3.43 (m, 2H), 2.00 (t, J=5.6 Hz, 4H), 0.61-0.40 (m, 4H).

### Intermediate 23

### 4-(cyclohexyloxy)-1-[6-(2-hydroxyphenyl)pyridazin-4-yl]piperidine-4-carboxylic acid

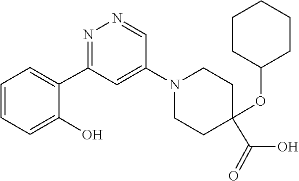 [SMILES: O=C(O)C1(OC2CCCCC2)CCN(c2cnnc(-c3ccccc3O)c2)CC1]

### Step 1: Synthesis of benzyl 4-(cyclohexyloxy)-4-(hydroxymethyl)piperidine-1-carboxylate

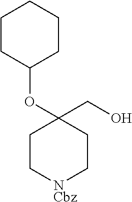 [SMILES: O=C(OCc1ccccc1)N1CCC(CO)(OC2CCCCC2)CC1]

A solution of benzyl 1-oxa-6-azaspiro[2.5]octane-6-carboxylate (6.0 g, 24.26 mmol) in cyclohexanol (50.523 ml, 485.252 mmol, 20.0 eq) was cooled down to +15° C. followed by dropwise addition of boron trifluoride diethyl etherate (6.88 g, 48.525 mmol, 2.0 eq). After addition was complete, the cooling bath was removed and stirring was continued for 1 h. DCM was added, followed by slow addition of water. The contents were transferred to a separatory funnel and the layers were separated. The aqueous layer was washed with DCM 3×. The combined organic extracts were dried over Na₂SO₄, filtered and concentrated under reduced pressure. The resulting residue was purified by RP-FC column for purification (330 g column, 80 mL flow, water/MeCN+0.1% FA, gradient 95:5 to 35:65) to afford benzyl 4-(cyclohexyloxy)-4-(hydroxymethyl)piperidine-1-carboxylate (3.68 g, 44%) as a colorless oil. LCMS: C₂₀H₂₉NO₄ requires 347.2. found: m/z=349.0 [M+H]+.

¹H NMR (300 MHz, DMSO-d6) δ 7.44-7.26 (m, 6H), 5.07 (s, 2H), 4.62 (t, J=5.2 Hz, 1H), 3.77-3.67 (m, 2H), 3.55 (br s, 1H), 3.15 (br s, J=19.2 Hz, 2H), 1.75-1.61 (m, 4H), 1.61-1.38 (m, 6H), 1.28-1.17 (m, 4H).

### Step 2: Synthesis of benzyl 4-(cyclohexyloxy)-4-formylpiperidine-1-carboxylate

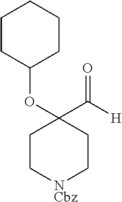 [SMILES: O=CC1(OC2CCCCC2)CCN(C(=O)OCc2ccccc2)CC1]

Dess-Martin periodinane, 95% (5.84 g, 13.76 mmol) was added to a stirring solution of benzyl 4-(cyclohexyloxy)-4-(hydroxymethyl)piperidine-1-carboxylate (3.68 g, 10.59 mmol) in dichloromethane (106 ml, 0.1 M) and the resulting mixture was stirred at room temperature for 2 h. Once complete, DCM (50 mL) and water (50 M) were added, followed by sat. aq. Na₂S₂O₃ (50 ml) and the mixture was stirred for 15 min. The contents were transferred to a separatory funnel and the layers were separated. The aqueous layer was washed with DCM 2× and the combined organic layers were washed with brine, collected, dried over Na₂SO₄, filtered and concentrated in vacuo to afford benzyl 4-(cyclohexyloxy)-4-formylpiperidine-1-carboxylate (3.65 g, 100%) as a colorless oil. LCMS: C₂₀H₂₇NO₄ requires 345.1. found: m/z=346.2 [M+H]+.

¹H NMR (300 MHz, DMSO-d6) δ 9.57 (s, 1H), 7.42-7.27 (m, 5H), 5.08 (s, 2H), 3.71 (dt, J=13.5, 4.5 Hz, 2H), 3.20 (d, J=11.3 Hz, 2H), 1.81-1.54 (m, 8H), 1.52-1.42 (m, 1H), 1.36-1.09 (m, 5H).

### Step 3: Synthesis of 1-[(benzyloxy)carbonyl]-4-(cyclohexyloxy)piperidine-4-carboxylic acid

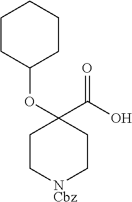 [SMILES: O=C(OCc1ccccc1)N1CCC(OC2CCCCC2)(C(=O)O)CC1]

A flask was charged with benzyl 4-(cyclohexyloxy)-4-formylpiperidine-1-carboxylate (3.65 g, 10.56 mmol) and potassium phosphate monobasic (4.31 g, 31.69 mmol). T-Butanol (106 ml), Water (26 ml) and 2-Methyl-2-butene (13.43 ml, 126.8 mmol) were then added sequentially. Sodium chlorite (3.58 g, 31.69 mmol) was added and the reaction mixture was stirred at room temperature for 2 h. Once complete by LCMS, the reaction was diluted with DCM and the pH was adjusted to ˜12 using 1M NaOH. The contents were transferred to a separatory funnel and the layers were separated. The aqueous layer was then acidified with 1 M HCl to pH˜2 and extracted with EtOAc 3×. The combined organic layers were dried over Na₂SO₄, filtered and evaporated to afford 1-[(benzyloxy)carbonyl]-4-(cyclohexyloxy)piperidine-4-carboxylic acid (0.77 g, 17%) containing 14 wt % t-butanol. LCMS: C₂₀H₂₇NO₅ requires 361.0. found: m/z=362.2 [M+H]+.

1H NMR (300 MHz, DMSO-d6) δ 12.81 (s, 1H), 7.44-7.24 (m, 5H), 5.07 (s, 2H), 3.67-3.56 (m, 2H), 1.77 (t, J=5.8 Hz, 6H), 1.65 (d, J=5.2 Hz, 2H), 1.50-1.38 (m, 1H), 1.35-1.14 (m, 6H), 1.12 (s, 9H).

### Step 4: Synthesis of 1-benzyl 4-methyl 4-(cyclohexyloxy)piperidine-1,4-dicarboxylate

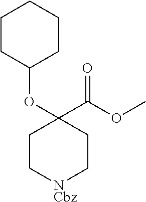 [SMILES: COC(=O)C1(OC2CCCCC2)CCN(C(=O)OCc2ccccc2)CC1]

(Trimethylsilyl)diazomethane, (2M in hexanes) (5.95 ml, 11.91 mmol) was added dropwise to a cooled (10° C.) solution of 1-[(benzyloxy)carbonyl]-4-(cyclohexyloxy)piperidine-4-carboxylic acid (0.77 g, 1.83 mmol) in methanol (7.33 ml, 0.25 M). Addition was continued until the light yellow color persisted in the solution. The cooling bath was then removed and the reaction was stirred at room temperature for 1 h. Once complete, acetic acid was added dropwise until the yellow color disappeared and the solution became colorless. Residual solvents were evaporated and the residue was purified by chromatography (hexane/MTBE, 100:0 to 75:25) to afford 1-benzyl 4-methyl 4-(cyclohexyloxy)piperidine-1,4-dicarboxylate (0.64 g, 93%) as a colorless oil.

¹H NMR (300 MHz, DMSO-d₆) δ 7.41-7.27 (m, 5H), 5.07 (s, 2H), 3.67 (s, 3H), 3.66-3.56 (m, 2H), 3.38 (m, 1H), 3.25 (br, 2H), 1.80 (t, J=4.5 Hz, 4H), 1.76-1.59 (m, 4H), 1.45 (d, J=10.7 Hz, 1H), 1.31-1.10 (m, 5H).

### Step 5: Synthesis of methyl 4-(cyclohexyloxy)piperidine-4-carboxylate

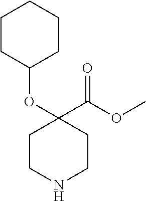 [SMILES: COC(=O)C1(OC2CCCCC2)CCNCC1]

1-benzyl 4-methyl 4-(cyclohexyloxy)piperidine-1,4-dicarboxylate (0.64 g, 1.70 mmol) was dissolved in THF (17 ml) in a 100 ml three-neck-RBF equipped with argon balloon and hydrogen balloon. The reaction mixture was degassed with argon and Pd/C (10% by weight; 0.18 g, 0.17 mmol) was added followed again by degassing with argon and filling with hydrogen. The reaction was stirred at room temperature overnight. The reaction mixture was filtered through a celite pad and evaporated to dryness to provide methyl 4-(cyclohexyloxy)piperidine-4-carboxylate (0.4 g, 92%) as a black solid.

¹H NMR (300 MHz, Chloroform-d) δ 4.04-3.89 (m, 1H), 3.75 (s, 3H), 3.38-3.16 (m, 4H), 2.26-2.17 (m, 2H), 2.01-1.92 (m, 2H), 1.73 (d, J=11.9 Hz, 5H), 1.27 (dd, J=24.7, 13.7 Hz, 6H).

### Step 6: Synthesis of methyl 1-(6-chloropyridazin-4-yl)-4-(cyclohexyloxy)piperidine-4-carboxylate

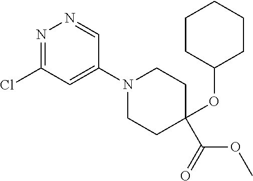 [SMILES: COC(=O)C1(OC2CCCCC2)CCN(c2cnnc(Cl)c2)CC1]

A mixture of 3,5-Dichloropyridazine (0.25 g, 1.73 mmol), methyl 4-(cyclohexyloxy)piperidine-4-carboxylate (0.40 g, 1.56 mmol) in DMSO (5.25 ml, 0.3 M) and N,N-diisopropylethylamine (1.65 ml, 9.45 mmol) was stirred under argon atmosphere in closed vial at 100° C. for 1 h. Once complete, the reaction was quenched with cold water and extracted with ethyl acetate 3×. The combined organic extracts were dried over Na₂SO₄, filtered and concentrated in vacuo to afford methyl 1-(6-chloropyridazin-4-yl)-4-(cyclohexyloxy)piperidine-4-carboxylate (0.50 g, 89%) as a sticky brown solid that was used in next step without additional purification. LCMS: C₁₇H₂₄ClN₃O₃ requires 353.1. found: m/z=354.2 [M+H]+.

¹H NMR (300 MHz, DMSO-d6) δ 8.96 (d, J=2.7 Hz, 1H), 7.10 (d, J=2.7 Hz, 1H), 3.72 (d, J=4.7 Hz, 1H), 3.68 (s, 4H), 3.39 (td, J=8.8, 4.2 Hz, 3H), 1.90 (q, J=4.5 Hz, 4H), 1.75-1.59 (m, 4H), 1.23 (q, J=11.5, 10.8 Hz, 6H).

### Step 7: Synthesis of methyl 4-(cyclohexyloxy)-1-{6-[2-(methoxymethoxy)phenyl]pyridazin-4-yl}piperidine-4-carboxylate

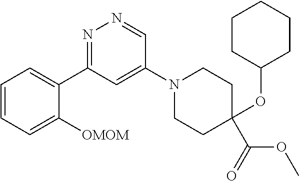

A sealed vial was charged with tetrakis(triphenylphosphine)palladium (0.162 g, 0.14 mmol), 2-(methoxymethoxy)phenylboronic acid (0.31 g, 1.67 mmol) and methyl 1-(6-chloropyridazin-4-yl)-4-(cyclohexyloxy)piperidine-4-carboxylate (0.5 g, 1.39 mmol) under argon atmosphere. 1,4-dioxane (13.99 ml, 0.1 M) was added followed by K₂CO₃ (0.112 g, 0.814 mmol) dissolved in H₂O (2.8 ml, 0.5 M). Argon was bubbled through the mixture for 20 min and the vial was sealed and stirred at 100° C. for 16 h. Once complete, the solvent was evaporated and the residue was purified by chromatography (EtOAc/hexane, 0 to 100%) to afford methyl 4-(cyclohexyloxy)-1-{6-[2-(methoxymethoxy)phenyl]pyridazin-4-yl}piperidine-4-carboxylate (0.52 g, 79%) as light yellow solid. LCMS: C₂₅H₃₃N₃O₅ requires 455.2. found: m/z=456.9 [M+H]⁺.

¹H NMR (300 MHz, DMSO-d₆) δ 8.96 (d, J=3.1 Hz, 1H), 7.62 (dd, J=7.6, 1.8 Hz, 1H), 7.42 (ddd, J=9.0, 7.4, 1.8 Hz, 1H), 7.27-7.19 (m, 2H), 7.12 (td, J=7.5, 1.1 Hz, 1H), 5.20 (s, 2H), 3.67 (s, 5H), 3.40 (d, J=9.5 Hz, 3H), 1.99-1.81 (m, 4H), 1.68 (d, J=11.5 Hz, 4H), 1.45 (d, J=11.2 Hz, 1H), 1.21 (d, J=20.7 Hz, 6H).

### Step 8: Synthesis of 4-(cyclohexyloxy)-1-{6-[2-(methoxymethoxy)phenyl]pyridazin-4-yl}piperidine-4-carboxylic acid

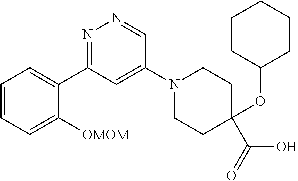

Methyl 4-(cyclohexyloxy)-1-{6-[2-(methoxymethoxy)phenyl]pyridazin-4-yl}piperidine-4-carboxylate (0.52 g, 1.10 mmol) was dissolved in dioxane (11.07 ml) and H₂O (4.43 mL) and lithium hydroxide monohydrate (0.232 g, 5.536 mmol, 5.0 eq) was added in one portion. The reaction mixture was stirred at room temperature for 16 h. Once complete, the mixture was neutralized with 4M HCl and evaporated in vacuo. The residue was purified using RP-FC (ACN/water) to afford 4-(cyclohexyloxy)-1-{6-[2-(methoxymethoxy)phenyl]pyridazin-4-yl}piperidine-4-carboxylic acid (0.45 g, 87%) as yellow solid.

LCMS: C₂₄H₃₁N₃O₅ requires 441.2. found: m/z=442.1 [M+H]+.

¹H NMR (300 MHz, DMSO-d6) δ 12.95 (br, 1H), 8.99 (d, J=3.1 Hz, 1H), 7.60 (dd, J=7.6, 1.7 Hz, 1H), 7.53 (t, J=7.9 Hz, 1H), 7.40 (s, 1H), 7.30 (d, J=8.4 Hz, 1H), 7.19 (t, J=7.5 Hz, 1H), 5.23 (s, 2H), 3.88 (s, 2H), 3.33 (s, 3H), 1.95 (d, J=13.3 Hz, 4H), 1.77 (s, 2H), 1.67 (s, 2H), 1.46 (s, 2H), 1.35-1.14 (m, 6H).

### Synthesis of Title Compound

4-(cyclohexyloxy)-1-{6-[2-(methoxymethoxy)phenyl]pyridazin-4-yl}piperidine-4-carboxylic acid (0.45 g, 0.968 mmol) was dissolved in dioxane (9.68 ml, 0.1 M) and hydrogen chloride (4.0M in dioxane) (7.26 ml, 29.05 mmol) was added in one portion. The reaction mixture was stirred at room temperature for 16 h. Once complete, the volatiles were evaporated and the residue was purified by RP-FC (ACN/water in 0.1% formic acid, 5%/95% to 75%/25%) to afford 4-(cyclohexyloxy)-1-[6-(2-hydroxyphenyl)pyridazin-4-yl]piperidine-4-carboxylic acid (0.25 g, 66%) as yellow solid. LCMS: C₂₂H₂₇N₃O₄ requires 397.2. found: m/z=398.2 [M+H]+.

¹H NMR (300 MHz, DMSO-d6) δ 13.71 (s, 1H), 8.95 (d, J=2.8 Hz, 1H), 8.18-8.04 (m, 1H), 7.57 (d, J=2.9 Hz, 1H), 7.33 (ddd, J=8.5, 7.2, 1.6 Hz, 1H), 7.01-6.84 (m, 2H), 3.95-3.76 (m, 2H), 3.48 (ddt, J=13.6, 8.6, 4.1 Hz, 3H), 2.02-1.61 (m, 8H), 1.26 (dt, J=21.9, 10.6 Hz, 6H).

### Intermediate 24

### 1-[6-(2-hydroxyphenyl)pyridazin-4-yl]-4-methoxypiperidine-4-carboxylic acid

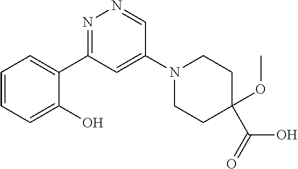 [SMILES: COC1(C(=O)O)CCN(c2cnnc(-c3ccccc3O)c2)CC1]

### Step 1: Synthesis of methyl 1-(6-chloropyridazin-4-yl)-4-methoxypiperidine-4-carboxylate

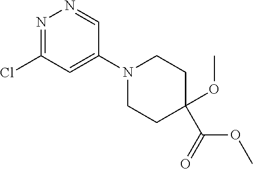 [SMILES: COC(=O)C1(OC)CCN(c2cnnc(Cl)c2)CC1]

A mixture of 3,5-Dichloropyridazine (0.71 g, 4.76 mmol), methyl-4-methoxypiperidine-4-carboxylate hydrochloride (1.00 g, 4.769 mmol) in N-methyl-2-pyrrolidone (9.54 ml, 0.5 M) and N,N-diisopropylethylamine (2.49 ml, 14.31 mmol) was stirred under argon atmosphere in closed vial at 100° C. for 1 h. Once complete, the reaction was quenched with cold water and then extracted with EtOAc 3×. The combined organic extracts were dried over Na₂SO₄, filtered and concentrated in vacuo to afford methyl 1-(6-chloropyridazin-4-yl)-4-methoxypiperidine-4-carboxylate (1.09 g, 63%) as a sticky brown solid that was used in next step without additional purification. LCMS: C₁₂H₁₆ClN₃O₃ requires 285.0. found: m/z=287.1 [M+H]+.

¹H NMR (300 MHz, DMSO-d6) δ 8.98 (d, J=2.8 Hz, 1H), 7.11 (d, J=2.7 Hz, 1H), 3.80 (dt, J=13.6, 4.2 Hz, 2H), 3.70 (s, 3H), 3.26 (m, 2H), 3.18 (s, 3H), 2.05-1.81 (m, 4H).

### Step 2: Synthesis of methyl 1-[6-(2-hydroxyphenyl)pyridazin-4-yl]-4-methoxypiperidine-4-carboxylate

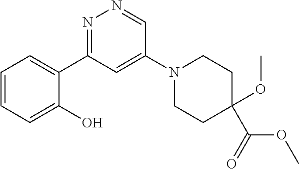 [SMILES: COC(=O)C1(OC)CCN(c2cnnc(-c3ccccc3O)c2)CC1]

Methyl 1-(6-chloropyridazin-4-yl)-4-methoxypiperidine-4-carboxylate (1.09 g, 3.02 mmol), K₂CO₃ (1.25 g, 9.06 mmol) and 2-hydroxyphenylboronic acid (0.625 g, 4.53 mmol) were dissolved in 1,4-dioxane (15.1 ml) and H₂O (3.02 ml) in a pressure vessel. The prepared solution was degassed with argon for 5 mins and tetrakis(triphenylphosphine)palladium (0.349 g, 0.302 mmol) was added in one portion. The reaction mixture was stirred for 18 h at 100° C. Once complete, the solvents were evaporated and the residue was purified by flash chromatography (Hexane:EtOAc; 0-100%) to provide methyl 1-[6-(2-hydroxyphenyl)pyridazin-4-yl]-4-methoxypiperidine-4-carboxylate (0.64 g, 54%) as a pale yellow solid. LCMS: C₁₈H₂₁N₃O₄ requires 343.1. found: m/z=344.6 [M+H]+.

¹H NMR (300 MHz, DMSO-d₆) δ 14.57 (s, 1H), 8.97 (d, J=2.9 Hz, 1H), 8.12 (dd, J=8.3, 1.6 Hz, 1H), 7.73-7.50 (m, 1H), 7.44-7.28 (m, 1H), 6.99-6.87 (m, 2H), 3.98 (dt, J=13.6, 4.2 Hz, 2H), 3.71 (s, 3H), 3.43-3.28 (m, 2H), 3.23 (s, 3H), 1.97 (p, J=4.2 Hz, 4H).

Following basic hydrolysis, the title compound was obtained (0.30 g, 55% yield) as a pale yellow solid. LCMS: C₁₇H₁₉N₃O₄ requires 329.1. found: m/z=330.1 [M+H]+.

¹H NMR (300 MHz, DMSO-d6) δ 13.67 (s, 2H), 8.97 (d, J=2.9 Hz, 1H), 8.17-8.07 (m, 1H), 7.58 (d, J=3.0 Hz, 1H), 7.34 (ddd, J=8.5, 7.2, 1.6 Hz, 1H), 6.99-6.87 (m, 2H), 3.97 (d, J=13.5 Hz, 2H), 3.32 (s, 2H), 3.25 (s, 3H), 1.99-1.89 (m, 4H).

### Intermediate 25

### 4-(2-chlorophenoxy)-1-[6-(2-hydroxyphenyl)pyridazin-4-yl]piperidine-4-carboxylic acid

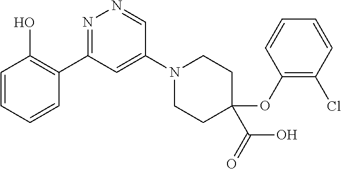 [SMILES: O=C(O)C1(Oc2ccccc2Cl)CCN(c2cnnc(-c3ccccc3O)c2)CC1]

### Step 1: 1-[(tert-butoxy)carbonyl]-4-(2-chlorophenoxy)piperidine-4-carboxylic acid

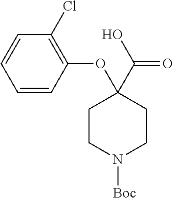 [SMILES: CC(C)(C)OC(=O)N1CCC(Oc2ccccc2Cl)(C(=O)O)CC1]

o-chlorophenol (2 g, 15.556 mmol, 1.0 equiv.) was dissolved in tetrahydrofuran anhydrous (27 mL, 0.5 M), cooled to 0° C. To this powdered NaOH (3.11 g, 77.784 mmol, 5.0 eq) and t-butyl 3-oxoazetidine-1-carboxylate (9.299 g, 46.670 mmol, 3.0 equiv.) were added to the solution at 0° C. Then chloroform (6.23 ml, 77.78 mmol, 5.0 equiv.) was added dropwise over the period 15 min and the reaction mixture was stirred for 1 h at 0° C., followed by overnight stirring at room temperature. The RM was diluted with water and extracted with diethyl ether (30 mL×3). The aqueous layer was acidified to pH=2 (using 2 M HCl) gave yellow precipitate. The yellow precipitate was dissolved in EtOAc and aqueous layer was extracted with EtOAc thrice. The organic phase was dried over MgSO₄, filtered and evaporated on rotavapor which afforded 1-[(tert-butoxy)carbonyl]-4-(2-chlorophenoxy)piperidine-4-carboxylic acid as yellow solid (5.1 g, 83%). LCMS (m/z): [M−H]⁻ calculated for C₁₇H₂₁ClNO₅⁻: 354.11. found: 354.95. ¹H NMR (300 MHz, DMSO-d₆) δ 13.49 (s, 1H), 7.47 (dd, J=7.9, 1.6 Hz, 1H), 7.26 (ddd, J=8.2, 7.4, 1.7 Hz, 1H), 7.00 (td, J=7.7, 1.3 Hz, 1H), 6.80 (dd, J=8.3, 1.4 Hz, 1H), 3.77 (d, J=13.3 Hz, 2H), 3.02 (s, 2H), 2.09 (d, J=14.0 Hz, 2H), 1.91 (ddd, J=14.0, 11.7, 4.6 Hz, 2H), 1.39 (s, 9H).

### Step 2: methyl 4-(2-chlorophenoxy)piperidine-4-carboxylate hydrochloride

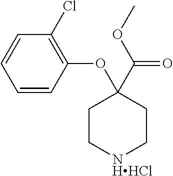

Thionyl chloride (1.684 ml, 23.22 mmol, 1.8 eq) was added carefully to a stirred solution of 232800123-VVI01-024 (5.1 g, 12.9 mmol, 1.0 eq) in Methanol anhydrous (16.12 ml, 0.8 M) at 0 C.

The resulting solution was stirred at reflux overnight. Next, it was cooled down to RT and volatiles were evaporated. The obtained solid was triturated with MTBE and filtered. The solid was collected and dried under reduced pressure and methyl 4-(2-chlorophenoxy)piperidine-4-carboxylate hydrochloride (4.0 g, 11.105 mmol, 86%) was obtained as an off white solid. LCMS (m/z): [M+H]+ calculated for C₁₃H₁₇ClNO₃⁺: 270.09. found: 270.50. ¹H NMR (300 MHz, DMSO-d₆) δ 9.18 (d, J=33.6 Hz, 2H), 7.53 (dd, J=8.0, 1.7 Hz, 1H), 7.29 (td, J=7.9, 1.7 Hz, 1H), 7.07 (td, J=7.7, 1.3 Hz, 1H), 6.75 (dd, J=8.3, 1.3 Hz, 1H), 3.79 (s, 3H), 3.25 (d, J=13.3 Hz, 2H), 2.96 (d, J=11.3 Hz, 2H), 2.31 (dq, J=10.7, 6.8, 5.4 Hz, 4H).

### Step 3: methyl 4-(2-chlorophenoxy)-1-(6-chloropyridazin-4-yl)piperidine-4-carboxylate

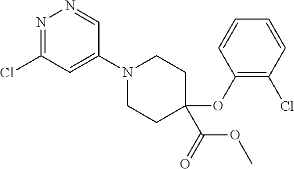 [SMILES: COC(=O)C1(Oc2ccccc2Cl)CCN(c2cnnc(Cl)c2)CC1]

A 150 mL pressure vial equipped with a magnetic stirrer was charged with 3,5-dichloropyridazine (0.993 g, 6.663 mmol, 1.2 eq) and methyl 4-(2-chlorophenoxy)piperidine-4-carboxylate hydrochloride (2.0 g, 5.552 mmol, 1.0 eq). To this dimethyl sulfoxide (11.1 ml, 0.5 M) and N,N-diisopropylethylamine (DIPEA) (5.803 ml, 33.314 mmol, 6.0 eq) were added. The reaction mixture was stirred at 100° C. overnight. UPLC indicated full conversion. DIPEA was evaporated on rotavapor, water was added and precipitate was obtained, sonicated and then brown solid was filtered off which was DP methyl 4-(2-chlorophenoxy)-1-(6-chloropyridazin-4-yl)piperidine-4-carboxylate (1.8 g, 3.814 mmol, 69%). LCMS (m/z): [M+H]⁺ calculated for C₁₇H₁₈Cl₂N₃O₃⁺: 382.07. found: 383.40. ¹H NMR (300 MHz, DMSO-d6) δ 8.98 (d, J=2.7 Hz, 1H), 7.50 (dd, J=7.9, 1.7 Hz, 1H), 7.28 (ddd, J=8.3, 7.4, 1.7 Hz, 1H), 7.18-6.98 (m, 2H), 6.77 (dd, J=8.3, 1.4 Hz, 1H), 3.93 (d, J=13.7 Hz, 2H), 3.77 (s, 3H), 3.27-3.11 (m, 2H), 2.25-2.03 (m, 4H).

### Step 4: methyl 4-(2-chlorophenoxy)-1-[6-(2-hydroxyphenyl)pyridazin-4-yl]piperidine-4-carboxylate

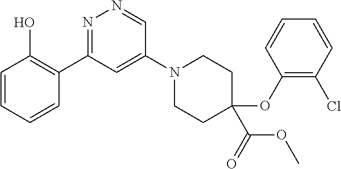 [SMILES: COC(=O)C1(Oc2ccccc2Cl)CCN(c2cnnc(-c3ccccc3O)c2)CC1]

In a pressure vial, methyl 4-(2-chlorophenoxy)-1-(6-chloropyridazin-4-yl)piperidine-4-carboxylate (1.7 g, 3.602 mmol, 1.0 eq), 2-hydroxyphenylboronic acid (0.547 g, 3.963 mmol, 1.1 eq), potassium carbonate anhydrous (1.494 g, 10.807 mmol, 3.0 eq), dioxane (36.02 ml, 0.1 M) and water (7.2 ml, 0.5 M) were added. The resulting reaction mixture was degassed with Ar for 30 min and then tetrakis(triphenylphosphine)palladium (0.416 g, 0.36 mmol, 0.1 eq) was added, stirred the reaction mixture at 100° C. for 10 h. UPLC showed full conversion. The crude mixture was purified by FC using eluent EtOAc/Cyc (0-60%) and methyl 4-(2-chlorophenoxy)-1-[6-(2-hydroxyphenyl)pyridazin-4-yl]piperidine-4-carboxylate (0.985 g, 2.15 mmol, 60%) was isolated as yellow solid. LCMS (m/z): [M+H]⁺ calculated for C₂₃H₂₃ClN₃O₄⁺: 440.14. found: 440.95. 1H NMR (300 MHz, DMSO-d₆) δ 14.50 (s, 1H), 8.98 (d, J=2.8 Hz, 1H), 8.09 (dd, J=8.5, 1.7 Hz, 1H), 7.59 (d, J=2.9 Hz, 1H), 7.51 (dd, J=8.0, 1.7 Hz, 1H), 7.37-7.25 (m, 2H), 7.06 (td, J=7.7, 1.3 Hz, 1H), 6.96-6.88 (m, 2H), 6.80 (dd, J=8.3, 1.4 Hz, 1H), 4.10 (d, J=13.6 Hz, 2H), 3.78 (s, 3H), 3.38 (d, J=4.5 Hz, 2H), 2.29-2.12 (m, 4H).

Following basic hydrolysis, the title compound was obtained (0.425 g, 0.979 mmol, 46%). LCMS (m/z): [M+H]⁺ calculated for C₂₂H₂₁ClN₃O₄⁺: 426.12. found: 426.19. ¹H NMR (300 MHz, DMSO-d₆) δ 14.02 (s, 2H), 8.98 (d, J=2.8 Hz, 1H), 8.11-8.02 (m, 1H), 7.59 (d, J=2.9 Hz, 1H), 7.50 (dd, J=7.9, 1.7 Hz, 1H), 7.32 (qd, J=7.6, 1.6 Hz, 2H), 7.03 (td, J=7.7, 1.3 Hz, 1H), 6.98-6.82 (m, 3H), 4.12 (d, J=13.4 Hz, 2H), 3.28 (d, J=4.2 Hz, 2H), 2.30-2.12 (m, 4H).

### Intermediate 26

### Synthesis of 1-[6-(2-hydroxyphenyl)pyridazin-4-yl]-4-phenyl-N-(piperidin-4-yl)-N-propylpiperidine-4-carboxamide

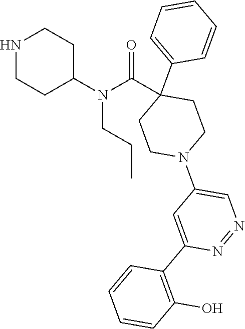 [SMILES: CCCN(C(=O)C1(c2ccccc2)CCN(c2cnnc(-c3ccccc3O)c2)CC1)C1CCNCC1]

Step 1: tert-butyl 4-(N-propyl1-{6-[2-(methoxymethoxy)phenyl]pyridazin-4-yl}-4-phenylpiperidine-4-amido)piperidine-1-carboxylate

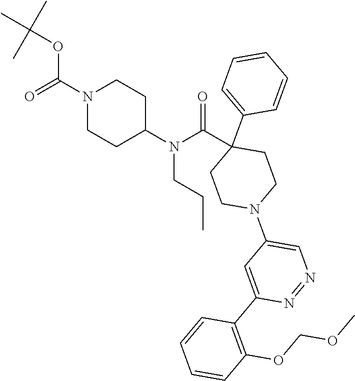 [SMILES: CCCN(C(=O)C1(c2ccccc2)CCN(c2cnnc(-c3ccccc3OCOC)c2)CC1)C1CCN(C(=O)OC(C)(C)C)CC1]

To a solution of 1-{6-[2-(methoxymethoxy)phenyl]pyridazin-4-yl}-4-phenylpiperidine-4-carboxylic acid (Intermediate 11) (0.6 g, 1.044 mmol, 1.0 eq) in dimethylacetamide (3.93 ml, 0.266 M) were added N,N-Diisopropylethylamine (DIPEA) (0.909 ml, 5.221 mmol, 5.0 eq), HATU (1.191 g, 3.132 mmol, 3.0 eq) and 1-Boc-4-propylaminopiperidine (1.518 g, 6.265 mmol, 6.0 eq). Mixture was stirred at 50° C. for 3 days. Then reaction mixture was diluted with DCM (50 mL) and extracted with NaHCO3 (50 mL). Basic phase was then extracted once again with DCM (50 mL). Combined organic layers were extracted with water (50 mL). Organic layer was dried over MgSO₄ and solvents were evaporated. Crude was purified by flash chromatography eluted by Hexane: EtOAc (0-70%) and then flushing with EtOAc:MeOH (9:1). Received impure product was purified by RP-FC to provide 0.36 g (0.531 mmol, 51% yield) of title compound. LCMS (m/z): [M+H]⁺ calculated for C₇H₄₉N₅O₅: 643.83. found: 644.84. ¹H NMR (300 MHz, DMSO-d₆) δ 8.92 (d, J=3.0 Hz, 1H), 7.61 (dd, J=7.6, 1.8 Hz, 1H), 7.46-7.36 (m, 3H), 7.33 (d, J=7.5 Hz, 2H), 7.28-7.20 (m, 2H), 7.17 (d, J=3.0 Hz, 1H), 7.12 (td, J=7.4, 1.1 Hz, 1H), 5.20 (s, 2H), 3.89 (d, J=13.1 Hz, 2H), 3.75 (d, J=12.4 Hz, 2H), 3.46 (s, 1H), 3.30 (s, 3H), 2.96 (d, J=8.3 Hz, 2H), 2.35 (d, J=13.6 Hz, 2H), 2.18 (t, J=12.5 Hz, 2H), 2.02 (s, 2H), 1.49-1.35 (m, 4H), 1.33 (s, 9H), 0.91-0.74 (m, 4H).

To the tert-butyl 4-(N-propyl1-{6-[2-(methoxymethoxy)phenyl]pyridazin-4-yl}-4-phenylpiperidine-4-amido)piperidine-1-carboxylate (0.36 g, 0.531 mmol, 1.0 eq) as a solution in DCM anh. (2.12 ml, 0.25 M) was added TFA (1.42 ml, 18.592 mmol, 35.0 eq). Reaction mixture was stirred for 2 h at room temperature. UPLC showed full conversion of starting material to desired product. Solvents were evaporated and received crude was triturated with diethyl ether twice. After drying 0.391 g of title compound was obtained (0.484 mmol, 91% yield). LCMS (m/z): [M+H]+ calculated for C₃₀H₃₇N₅O₂: 499.66. found: 500.71. ¹H NMR (300 MHz, DMSO-d₆) δ 8.95 (d, J=3.0 Hz, 1H), 8.66 (s, 1H), 8.31 (s, 1H), 7.59 (dd, J=7.9, 1.6 Hz, 1H), 7.46 (td, J=8.0, 7.5, 1.8 Hz, 3H), 7.41 (d, J=3.5 Hz, 2H), 7.33 (q, J=8.9, 6.9 Hz, 2H), 7.09 (d, J=8.2 Hz, 1H), 7.06-6.98 (m, 1H), 4.14 (s, 3H), 3.68-3.56 (m, 4H), 3.19-3.08 (m, 2H), 2.99 (t, J=7.9 Hz, 2H), 2.44 (s, 3H), 2.21 (s, 2H), 1.87-1.61 (m, 2H), 1.57-1.39 (m, 2H), 0.97-0.89 (m, 2H), 0.84 (t, J=7.3 Hz, 2H).

### Intermediate 27

### Synthesis of N-cyclopropyl-1-[6-(2-hydroxyphenyl)pyridazin-4-yl]-4-phenyl-N-(piperidin-4-yl)piperidine-4-carboxamide

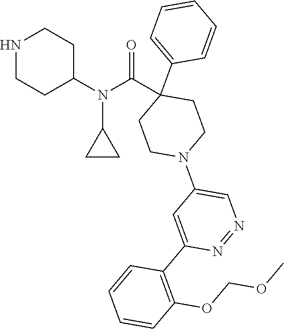 [SMILES: COCOc1ccccc1-c1cc(N2CCC(C(=O)N(C3CCNCC3)C3CC3)(c3ccccc3)CC2)cnn1]

The Title compounds was made in analogous fashion to Synthesis of 1-[6-(2-hydroxyphenyl)pyridazin-4-yl]-4-phenyl-N-(piperidin-4-yl)-N-propylpiperidine-4-carboxamide (Intermediate 26) by replacing 1-Boc-4-propylaminopiperidine with 1-Boc-4-cyclopropylaminopiperidine. (0.118 g 94% yield) of title compound was obtained. LCMS (m/z): [M+H]⁺ calculated for C₃₀H₃₅N₅O₂: 497.64. found: 498.65. 1H NMR (300 MHz, DMSO-d6) δ 8.70 (d, J=3.1 Hz, 1H), 7.55 (ddd, J=7.8, 5.4, 1.7 Hz, 2H), 7.51-7.45 (m, 2H), 7.45-7.36 (m, 3H), 7.34 (d, J=3.2 Hz, 1H), 7.18-7.08 (m, 2H), 4.10 (d, J=69.1 Hz, 2H), 3.89-3.48 (m, 3H), 3.33 (d, J=27.4 Hz, 2H), 2.75-2.50 (m, 2H), 2.36 (s, 5H), 2.12 (s, 2H), 1.56-1.24 (m, 2H), 0.91 (s, 2H), 0.64 (s, 2H).

### Intermediate 28

### Synthesis of rac-2-(1-(4-(2,6-dioxopiperidin-3-yl)phenyl)piperidin-4-yl)acetaldehyde

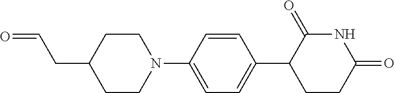 [SMILES: O=CCC1CCN(c2ccc(C3CCC(=O)NC3=O)cc2)CC1]

### Step 1: Synthesis of 2,6-bis(benzyloxy)-3-(4-bromophenyl)pyridine

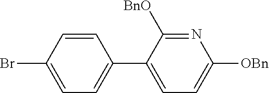 [SMILES: Brc1ccc(-c2ccc(OCc3ccccc3)nc2OCc2ccccc2)cc1]

To a solution 2,6-bis(benzyloxy)-3-(4,4,5,5-tetramethyl-1,3,2-dioxaborolan-2-yl)pyridine (65.0 g, 155 mmol), 1-bromo-4-iodobenzene (52.8 g, 186 mmol) in 1,4-dioxane (650 mL) and H₂O (165 mL) was added in K₂CO₃ (43.0 g, 311 mmol) and Pd(dppf)Cl₂ (11.4 g, 15.5 mmol) at 20° C. and purged with N₂ for 3 times. The mixture was heated and stirred at 80° C. until complete as judged by LCMS. Once complete, the reaction mixture was cooled down to room temperature and filtered through celite, with EtOAc rinsing. The collected filtrate was extracted with EtOAc (100 mL×3). The combined organic layers were washed with brine (500 mL), dried over anhydrous Na₂SO₄, filtered and concentrated to give a crude residue. The crude residue was purified by flash-column chromatography (petroleum ether in ethyl acetate=0 to 100%) to afford desired product, which was then triturated by petroleum ether in ethyl acetate (50:1, 500 mL) at 25° C. for 15 mins to obtain the desired compound as a white solid. (163 g, 80% yield). LCMS C₂₅H₂₀BrNO₂ requires 447.1, found m/z=448.2 [M+H]⁺.

### Step 2: Synthesis of ethyl 2-(1-(4-(2,6-bis(benzyloxy)pyridin-3-yl)phenyl)piperidin-4-yl)acetate

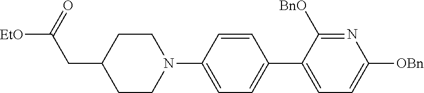 [SMILES: CCOC(=O)CC1CCN(c2ccc(-c3ccc(OCc4ccccc4)nc3OCc3ccccc3)cc2)CC1]

To a solution of 2,6-bis(benzyloxy)-3-(4-bromophenyl)pyridine (67.0 g, 146 mmol), ethyl 2-(piperidin-4-yl)acetate (37.6 g, 219 mmol) in 2-MeTHF (670 mL) and H₂O (67 mL) was added in DavePhos (11.5 g, 29.3 mmol), Cs₂CO₃ (143 g, 439 mmol) and Pd₂(dba)₃ (13.4 g, 14.6 mmol) and purged with N₂ (3×). The mixture was stirred at 100° C. until complete as judged by LCMS. Once complete, the mixture was poured into H₂O (1000 mL) and extracted with 2-MeTHF (800 mL×3). The combined organic layers were washed with brine, dried with anhydrous Na₂SO₄, filtered, and concentrated to afford a crude residue. The residue was purified by flash column chromatography (petroleum ether in ethyl acetate=0 to 100%) to afford purified material, which was triturated with MTBE (200 mL) at 20° C. for 30 mins to afford the desired product as a light-yellow solid was obtained as a light-yellow solid (76.3 g, 45% yield). LCMS C₃₄H₃₆N₂O₄ requires 536.3, found m/z=537.4 [M+H]⁺.

### Step 3: Synthesis of 2-(1-(4-(2,6-bis(benzyloxy)pyridin-3-yl)phenyl)piperidin-4-yl)ethan-1-ol

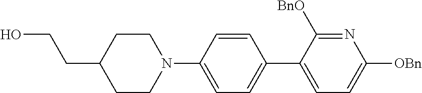 [SMILES: OCCC1CCN(c2ccc(-c3ccc(OCc4ccccc4)nc3OCc3ccccc3)cc2)CC1]

Flow Procedure: Solution 1: ethyl 2-(1-(4-(2,6-bis(benzyloxy)pyridin-3-yl)phenyl)piperidin-4-yl)acetate (1.00 eq, 76.0 g in THF, 760 mL). Solution 2: LiAlH₄ (2.00 eq, 0.707 g, 114 mL, 2.50 M). The whole flow-reaction process is performed under an inert atmosphere of N₂. The volume of flow reactor 1⅛″ PFA coil was 60 mL. The residence time of flow reactor 1 was 2 min. Set the bath at 20° C. for flow reactor 1. The flow rate of Pump 1 was adjusted to 26.1 mL/min for solution 1. The flow rate of Pump 2 was adjusted to 3.9 mL/min for solution 2. The mixture was collected with a bottle quenched by Na₂SO₄-10H₂O. The Pump 1 and Pump 2 was started and the reaction mixture was collected after running 5 mins. Once the reaction was complete as judged by LCMS, the reaction mixture was filtered through celite to remove Na₂SO₄ with THF (500 mL) and DCM (500 mL) rinsing. The crude filtrate was concentrated by vacuum to afford desired product as a gray solid, which was taken forward without any further purification (67.7 g). LCMS C₃₂H₃₄N₂O₃ requires 494.3, found m/z=495.3 [M+H]⁺.

### Step 4: Synthesis of rac-3-(4-(4-(2-hydroxyethyl)piperidin-1-yl)phenyl)piperidine-2,6-dione

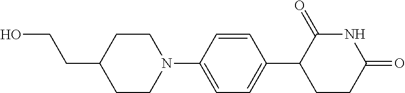 [SMILES: O=C1CCC(c2ccc(N3CCC(CCO)CC3)cc2)C(=O)N1]

An oven-dried flask was purged under vacuum and by backfilled with N₂ (3×), followed by addition of Pd/C (14.2 g, 66.7 mmol, 50% purity), Pd(OH)₂ (14.2 g, 50.5 mmol, 50% purity), 2-(1-(4-(2,6-bis(benzyloxy)pyridin-3-yl)phenyl)piperidin-4-yl)ethan-1-ol (74.0 g, 149 mmol) and THF (740 mL) was added in the bottle. The suspension was purged of N₂ and charged with H₂ (3×). The reaction mixture was stirred under H₂ (50 psi) at 25° C. until complete as judged by LCMS. Once complete, the mixture was filtered with THF rinsing (2.00 L). The collected filtrate was refluxed at 80° C. for 15 minutes and then filtered while hot to obtain the filtrate. The collected filtrate was concentrated give a crude residue that was triturated by MTBE (60 mL) to afford the desired compound as white solid, which was taken forward without any further purification (40.0 g). LCMS C₁₈H₂₄N₂O₃ requires 316.2, found m/z=317.2 [M+H]⁺.

### Step 5: Synthesis of Title Compound

An oven dried vial was purged and then backfilled with N₂ (3×), followed by addition of a solution of rac-(R)-3-(4-(4-(2-hydroxyethyl)piperidin-1-yl)phenyl)piperidine-2,6-dione (30.0 g, 94.8 mmol) in DMSO (300 mL) at 25° C. IBX (39.0 g, 139 mmol, 1.47 eq) was then added portion-wise, and the mixture was allowed to stir at 25° C. until complete as judged by LCMS. Once complete, the mixture was cooled to 15° C. and 2-MeTHF (300 mL) was added to prevent the precipitation of products during quenching of the reaction. H₂O (500 mL) was added to quench the reaction, and the reaction mixture was then extracted by 2-MeTHF (300 mL×3) and DCM (800 mL×10). The combined organic layers were washed by brine (500 mL) and saturated NaHCO₃ solution (500 mL), dried via anhydrous Na₂SO₄, filtered and concentrated to give a crude residue. The residue was purified by flash column chromatography (Petroleum ether/Ethyl acetate (w/10% DCM)=0-33%) to afford the desired product as a green solid (26.0 g, 84% yield). LCMS C₁₈H₂₂N₂O₃ requires 314.2, found m/z=315.3 [M+H]⁺.

### Intermediate 29

### (S)-2-(1-(4-(2,6-dioxopiperidin-3-yl)phenyl)piperidin-4-yl)acetaldehyde

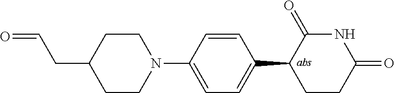 [SMILES: O=CCC1CCN(c2ccc([C@@H]3CCC(=O)NC3=O)cc2)CC1]

rac-2-(1-(4-(2,6-dioxopiperidin-3-yl)phenyl)piperidin-4-yl)acetaldehyde (24.9 g, 79.4 mmol) was separated by SFC (column: DAICEL CHIRALPAK AS (250 mm*30 mm, 10 um); mobile phase: [CO₂-i-PrOH/ACN]; B %: 70%, isocratic elution mode). The title compound (first-eluting isomer) was obtained as a yellow solid (7.00 g, 44.6% yield). LCMS C₁₈H₂₂N₂O₃ requires 314.2, found m/z=315.3 [M+H]⁺.

### Intermediate 30

### rac-3-((4-(piperidin-4-yl)phenyl)amino)piperidine-2,6-dione

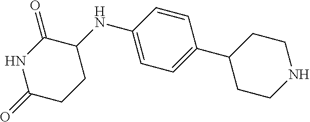 [SMILES: O=C1CCC(Nc2ccc(C3CCNCC3)cc2)C(=O)N1]

### Step 1: Synthesis of rac-tert-butyl (R)-4-(4-((2,6-dioxopiperidin-3-yl)amino)phenyl)piperidine-1-carboxylate

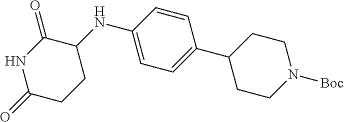 [SMILES: CC(C)(C)OC(=O)N1CCC(c2ccc(NC3CCC(=O)NC3=O)cc2)CC1]

To a solution of tert-butyl 4-(4-aminophenyl)piperidine-1-carboxylate (23.5 g, 85.0 mmol, 1.0 eq) and NaHCO₃ (21.4 g, 255.0 mmol, 3.0 eq) in DMF (250 mL) was added rac-3-bromopiperidine-2,6-dione (24.5 g, 127.5 mmol, 1.5 eq) at room temperature and the reaction mixture was stirred at 65° C. until complete as judged by LCMS. Once complete, the mixture was quenched with ice water (500 mL) and stirred at room temperature for 15 min. The reaction mixture was filtered and concentrated to give crude product. The crude product was triturated with MTBE (150 mL) to give desired product as a gray solid which was taken forward without any further purification (26 g). LCMS C₂₁H₂₉N₃O₄ requires 387.2, found 386.1 [M−H]⁻.

### Step 2: Synthesis of Title Compound

To a solution of rac-tert-butyl-4-(4-((2,6-dioxopiperidin-3-yl)amino)phenyl)piperidine-1-carboxylate (22.5 g, 58.1 mmol) in DCM (66 mL) was added 4N HCl in dioxane (43.6 mL, 174.3 mmol) at 0° C. and the reaction mixture was stirred at room temperature until complete as judged by LCMS. Once complete, the mixture was concentrated to give crude product. The crude product was triturated with MTBE (100 mL) to give the desired product as a grey solid, which was taken forward without any further purification (16.0 g, HCl salt). LCMS C₁₆H₂₁N₃O₂ requires 287.2, found 288.2 [M+H]⁺.

### Intermediate 31

### (1R,4R)-4-(4-((RS)-2,6-dioxopiperidin-3-yl)phenoxy)cyclohexane-1-carbaldehyde

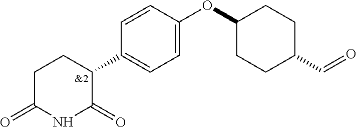 [SMILES: O=C[C@H]1CC[C@H](Oc2ccc([C@H]3CCC(=O)NC3=O)cc2)CC1]

### Step 1: Synthesis of methyl (1r,4r)-4-(4-bromophenoxy)cyclohexane-1-carboxylate

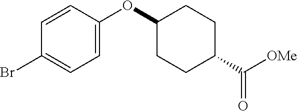 [SMILES: COC(=O)[C@H]1CC[C@H](Oc2ccc(Br)cc2)CC1]

To a solution of methyl (1r,4r)-4-hydroxycyclohexane-1-carboxylate (20.0 g, 126 mmol) in toluene (400 mL) was added PPh₃ (36.4 g, 139 mmol) and 4-bromophenol (24.0 g, 139 mmol). The mixture was then cooled to 0° C. and to the mixture was added DEAD (27.5 mL, 151 mmol). The reaction mixture was then allowed to warm to 25° C. and react until complete as judged by LCMS. Once complete, the reaction mixture was poured into petroleum ether (1000 mL) where a precipitate formed. The mixture was filtered, and the filter cake was washed with petroleum ether (200 mL×2).

The combined filtrate was washed with brine (500 mL×3), dried over anhydrous Na2SO4, filtered, and concentrated under vacuum to afford a crude residue. The residue was purified by column chromatography (petroleum ether in ethyl acetate=0 to 100%) to afford the desired compound as a white solid (23.0 g, 58% yield over two steps). LCMS C₁₄H₁₇BrO₃ requires 312.1, found m/z=313.2 [M+H]⁺.

### Step 2: Synthesis of methyl (1r,4r)-4-(4-(2,6-bis(benzyloxy)pyridin-3-yl)phenoxy)cyclohexane-1-carboxylate

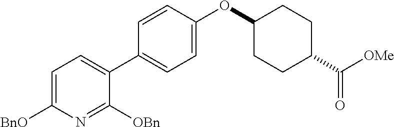 [SMILES: COC(=O)[C@H]1CC[C@H](Oc2ccc(-c3ccc(OCc4ccccc4)nc3OCc3ccccc3)cc2)CC1]

This intermediate was prepared as described for intermediate 28 Step 1, using methyl (1r,4r)-4-(4-bromophenoxy)cyclohexane-1-carboxylate in place of 1-bromo-4-iodobenzene (12.0 g, 72% yield). LCMS C₃₃H₃₃NO₅ requires 523.2, found m/z=524.2 [M+H]⁺.

### Step 3: Synthesis of ((1r,4r)-4-(4-(2,6-bis(benzyloxy)pyridin-3-yl)phenoxy)cyclohexyl)methanol

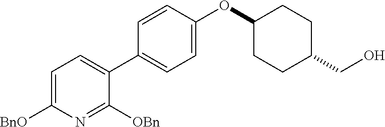 [SMILES: OC[C@H]1CC[C@H](Oc2ccc(-c3ccc(OCc4ccccc4)nc3OCc3ccccc3)cc2)CC1]

To a solution of (1r,4r)-4-(4-(2,6-bis(benzyloxy)pyridin-3-yl)phenoxy)cyclohexane-1-carboxylate (6.00 g, 11.4 mmol) in THF (60 mL) was added LiBH₄ (2.00 M in THF, 236 mL) at 0° C. under N₂. The mixture was warmed to 25° C. and allowed to react until complete as judged by LCMS. Once complete, the reaction mixture was cooled down to 0° C. and to the mixture was added a 5% solution of NaHCO₃ (100 mL) at 0° C., slowly. The mixture was extracted with ethyl acetate (150 mL×3). The combined organic layers were washed with brine (150 mL×2), dried over anhydrous Na₂SO₄, filtered, and concentrated to give the desired compound as a light-yellow solid which was taken forward without any further purification (5.0 g, 87% yield). LCMS C₃₂H₃₃NO₄ requires 495.2, found m/z=496.3 [M+H]⁺.

### Step 4: Synthesis of (RS)-3-(4-(((1r,4R)-4-(hydroxymethyl)cyclohexyl)oxy)phenyl)piperidine-2,6-dione

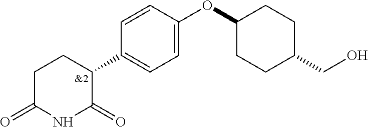 [SMILES: O=C1CC[C@H](c2ccc(O[C@H]3CC[C@H](CO)CC3)cc2)C(=O)N1]

To a solution of ((1r,4r)-4-(4-(2,6-bis(benzyloxy)pyridin-3-yl)phenoxy)cyclohexyl)methanol (3.00 g, 5.95 mmol) in THF (30 mL) was added Pd/C (600 mg, 10% purity), Pd(OH)₂ (600 mg, 20% purity) under N₂ atmosphere. The suspension was degassed and purged with H₂ 3 times. The mixture was stirred under H₂ (15 Psi) at 25° C. until complete as judged by LCMS. Once complete, the reaction mixture was filtered through a layer of Celite, with THF washing. The collected filtrate was concentrated under vacuum to give the desired product as yellow solid which was taken forward without any further purification (2.50 g). LCMS C₁₈H₂₃NO₄ requires 317.2, found m/z=318.3 [M+H]⁺.

### Step 5: Synthesis of Title Compound

To a solution of (RS)-3-(4-(((1r,4R)-4-(hydroxymethyl)cyclohexyl)oxy)phenyl)piperidine-2,6-dione (1.0 g, 3.04 mmol) in DMSO (10.0 mL) was slowly added DMP (2.58 g, 6.09 mmol) at 25° C. The reaction was stirred at 25° C. until complete as judged by LCMS. Once complete, the mixture was adjusted with saturated aqueous Na₂CO₃ to pH=10 and the aqueous layer was extracted with ethyl acetate (50 mL×3). The combined organic layers were washed with half-saturated Na₂S₂O₃ solution (50 mL), brine (50 mL), dried over anhydrous Na₂SO₄, filtered, and concentrated under vacuum to afford a crude residue. The crude product was triturated with Petroleum ether/ethyl acetate=(3:1, 5 mL) at 25° C. for 15 mins to give to give the desired product as a white solid, which was taken forward without any further purification (0.66 g). LCMS C₁₈H₂₁NO₄ requires 315.2, found m/z=316.2 [M+H]⁺.

### Intermediate 32

### rac-(R)-2-(4-(4-(2,6-dioxopiperidin-3-yl)phenyl)-1-oxa-4,9-diazaspiro[5.5]undecan-9-yl)acetic acid

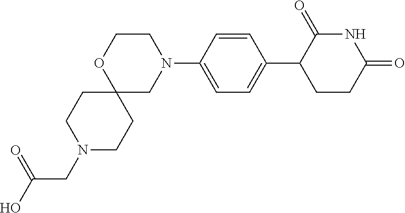 [SMILES: O=C(O)CN1CCC2(CC1)CN(c1ccc(C3CCC(=O)NC3=O)cc1)CCO2]

### Step 1: Synthesis of tert-butyl 4-(4-(2,6-bis(benzyloxy)pyridin-3-yl)phenyl)-1-oxa-4,9-diazaspiro[5.5]undecane-9-carboxylate

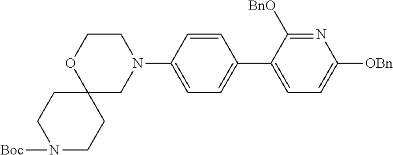 [SMILES: CC(C)(C)OC(=O)N1CCC2(CC1)CN(c1ccc(-c3ccc(OCc4ccccc4)nc3OCc3ccccc3)cc1)CCO2]

To a stirred solution of 2,6-bis(benzyloxy)-3-(4-bromophenyl)pyridine (5 g, 11.20 mmol) and tert-butyl 1-oxa-4,9-diazaspiro[5.5]undecane-9-carboxylate (2.87 g, 11.20 mmol) in 1,4 dioxane (50 mL) was added cesium carbonate (7.30 g, 22.40 mmol) at room temperature. The resulting mixture was purged with N₂ for 10 min. Then Pd₂(dba)₃ (0.513 g, 0.560 mmol) and RuPhos (0.523 g, 1.120 mmol) were added. The resulting mixture was purged with N₂ for 5 min and heated to 100° C. for 16 h. Upon completion of reaction, the reaction mixture was cooled to RT and filtered through Celite, with ethyl acetate rinsing (300 mL). The filtrate was diluted with water (200 mL) and extracted with ethyl acetate (2×200 mL). The combined organic layer was dried over anhydrous sodium sulphate and concentrated under vacuum to afford the crude compound as a brown liquid. The crude compound was purified by flash-column chromatography using ethyl acetate/pet-ether (0-30%) as the eluent to afford the desired compound as pale yellow solid. (5.6 g, 77% yield). LCMS C₃₈H₄₃N₃O₅ requires 621.3, found m/z=622.2 [M+H]⁺.

### Step 2: Synthesis of 4-(4-(2,6-bis(benzyloxy)pyridin-3-yl)phenyl)-1-oxa-4,9-diazaspiro[5.5]undecane

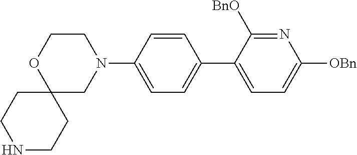 [SMILES: c1ccc(COc2ccc(-c3ccc(N4CCOC5(CCNCC5)C4)cc3)c(OCc3ccccc3)n2)cc1]

This intermediate was prepared as described for the synthesis of intermediate 30 Step 2, using tert-butyl 4-(4-(2,6-bis(benzyloxy)pyridin-3-yl)phenyl)-1-oxa-4,9-diazaspiro[5.5]undecane-9-carboxylate in place of rac-tert-butyl (R)-4-(4-((2,6-dioxopiperidin-3-yl)amino)phenyl)piperidine-1-carboxylate (4.5 g, HCl salt). LCMS C₃₃H₃₅N₃O₃ requires 521.3, found 522.4 [M+H]⁺.

### Step 3: Synthesis of tert-butyl 2-(4-(4-(2,6-bis(benzyloxy)pyridin-3-yl)phenyl)-1-oxa-4,9-diazaspiro[5.5]undecan-9-yl)acetate

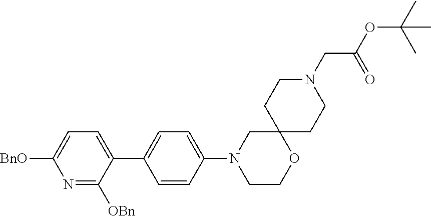 [SMILES: CC(C)(C)OC(=O)CN1CCC2(CC1)CN(c1ccc(-c3ccc(OCc4ccccc4)nc3OCc3ccccc3)cc1)CCO2]

To a solution of (4-(4-(2,6-bis(benzyloxy)pyridin-3-yl)phenyl)-1-oxa-4,9-diazaspiro[5.5]undecane (4.5 g, 7.33 mmol) in MeCN (50 mL) at RT was added TEA (5.11 ml, 36.7 mmol) and tert-butyl 2-chloroacetate (3.15 mL, 22.00 mmol). The reaction mixture was stirred at 60° C. until complete as judged by LCMS. Upon completion of reaction, the reaction mixture was cooled to room temperature. Once cooled, water (100 mL) was added, and the mixture was extracted with EtOAc (2×250 mL). The combined organic layers were washed with water (200 mL), brine (100 mL), dried over anhydrous Na₂SO₄ and concentrated under reduced pressure to give a crude product. The crude product was purified by FCC using 30% ethyl acetate/pet-ether as eluent (isocratic elution) to afford the desired product as an off-white solid (3.0 g, 61% yield). LCMS C₃₉H₄₅N₃O₅ requires 636.3, found m/z=637.4 [M+H]⁺.

### Step 4: Synthesis of rac-tert-butyl (R)-2-(4-(4-(2,6-dioxopiperidin-3-yl)phenyl)-1-oxa-4,9-diazaspiro[5.5]undecan-9-yl)acetate

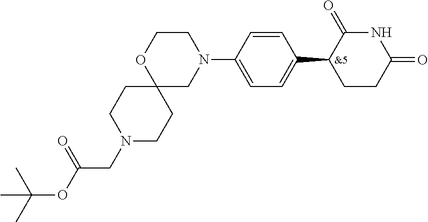 [SMILES: CC(C)(C)OC(=O)CN1CCC2(CC1)CN(c1ccc([C@@H]3CCC(=O)NC3=O)cc1)CCO2]

To a stirred solution of tert-butyl 2-(4-(4-(2,6-bis(benzyloxy)pyridin-3-yl)phenyl)-1-oxa-4,9-diazaspiro[5.5]undecan-9-yl)acetate (3 g, 4.72 mmol) in 1,4-dioxane (30 mL) was added Pd(OAc)₂ (0.4 g, 1.782 mmol) and 10% Pd/C (0.5 g, 4.70 mmol) under N₂ atmosphere at RT. The resulting mixture was stirred under H₂ pressure (1 atm) at RT until complete as judged by LCMS. Once complete, the reaction mixture was filtered through celite with ethyl acetate washing (100 mL). The eluent was concentrated under reduced pressure to afford crude compound, which was washed with toluene (2×50 mL), MTBE (2×30 mL) and concentrated under reduced pressure to afford the desired compound as an off-white solid which was taken forward without any further purification (1.8 g). LCMS C₂₅H₃₅N₃O₅ requires 457.3, found 458.6 [M+H]⁺.

### Step 5: Synthesis of Title Compound

This intermediate was prepared as described for the synthesis of Intermediate 30 step 2, using rac-tert-butyl (R)-2-(4-(4-(2,6-dioxopiperidin-3-yl)phenyl)-1-oxa-4,9-diazaspiro[5.5]undecan-9-yl)acetate in place of rac-tert-butyl (R)-4-(4-((2,6-dioxopiperidin-3-yl)amino)phenyl)piperidine-1-carboxylate (0.91 g, HCl salt). LCMS C₂₁H₂₇N₃O₅ requires 401.2, found 402.2 [M+H]⁺.

### Intermediate 33

### 2-((3ar,6ar)-5-(4-(2,6-dioxopiperidin-3-yl)phenyl)hexahydropyrrolo[3,4-c]pyrrol-2(1h)-yl)acetic acid

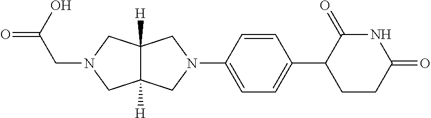 [SMILES: O=C(O)CN1C[C@@H]2CN(c3ccc(C4CCC(=O)NC4=O)cc3)C[C@H]2C1]

### Step 1: Synthesis of tert-butyl (3aS,6aS)-5-(4-(2,6-bis(benzyloxy)pyridin-3-yl)phenyl)hexahydropyrrolo[3,4-c]pyrrole-2(1H)-carboxylate

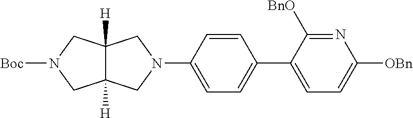 [SMILES: CC(C)(C)OC(=O)N1C[C@@H]2CN(c3ccc(-c4ccc(OCc5ccccc5)nc4OCc4ccccc4)cc3)C[C@H]2C1]

To a stirred solution of 2,6-bis(benzyloxy)-3-(4-bromophenyl)pyridine (5 g, 11.20 mmol) and tert-butyl (3aS,6aS)-hexahydropyrrolo[3,4-c]pyrrole-2(1H)-carboxylate (2.378 g, 11.20 mmol) in dioxane (60 mL) was added cesium carbonate (7.30 g, 22.40 mmol) at room temperature. The resulting mixture was purged with N₂ for 10 min. Then Pd₂(dba)₃ (0.513 g, 0.560 mmol) and RuPhos (0.523 g, 1.120 mmol) were added. The resulting mixture was purged with N₂ for 5 min and heated to 100° C. for 16 h. Upon completion of reaction, as confirmed by LCMS, the reaction mixture was cooled to RT and filtered through Celite pad and washed with ethyl acetate (300 mL). The filtrate was diluted with water (200 mL) and extracted with ethyl acetate (2×200 mL). The combined organic layer was dried over sodium sulphate and concentrated under reduced pressure to afford the crude product as brown liquid. It was purified by flash chromatography with a gradient of 0-30% ethyl acetate/pet-ether to afford tert-butyl (3aS,6aS)-5-(4-(2,6-bis(benzyloxy)pyridin-3-yl)phenyl)hexahydropyrrolo[3,4-c]pyrrole-2(1H)-carboxylate (4.5 g, 66% yield) as pale-yellow solid. LCMS: C₃₆H₃₉N₃O₄ requires 577.3. found: m/z=578.2 [M+H]⁺.

### Step 2: Synthesis of (3aR,6aR)-2-(4-(2,6-bis(benzyloxy)pyridin-3-yl)phenyl)octahydropyrrolo[3,4-c]pyrrole

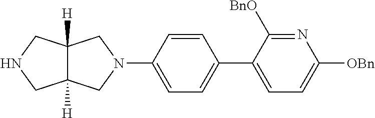 [SMILES: c1ccc(COc2ccc(-c3ccc(N4C[C@H]5CNC[C@@H]5C4)cc3)c(OCc3ccccc3)n2)cc1]

To a stirred solution of tert-butyl (3aS,6aS)-5-(4-(2,6-bis(benzyloxy)pyridin-3-yl)phenyl)hexahydropyrrolo[3,4-c]pyrrole-2(1H)-carboxylate (4.5 g, 7.79 mmol) in DCM (20 mL) was added 4M HCl in dioxane (9.74 ml, 38.9 mmol) at 0° C. The resulting reaction mixture was stirred at RT for 2 h. Upon completion of reaction, as confirmed by LCMS, the reaction mixture was concentrated under reduced pressure and washed with n-hexane (3×100 mL) and dried to afford (3aR,6aR)-2-(4-(2,6-bis(benzyloxy)pyridin-3-yl)phenyl)octahydropyrrolo[3,4-c]pyrrole (3.5 g, 70% yield) as off-white solid. LCMS: C₃₁H₃₁N₃O₂ requires 477.2. found: m/z=478.2 [M+H]⁺.

### Step 3: Synthesis of tert-butyl 2-((3aR,6aR)-5-(4-(2,6-bis(benzyloxy)pyridin-3-yl)phenyl)hexahydropyrrolo[3,4-c]pyrrol-2(1H)-yl)acetate

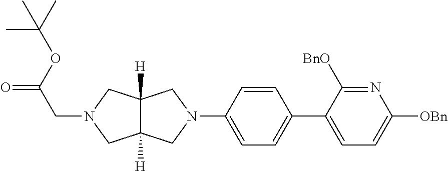 [SMILES: CC(C)(C)OC(=O)CN1C[C@@H]2CN(c3ccc(-c4ccc(OCc5ccccc5)nc4OCc4ccccc4)cc3)C[C@H]2C1]

To a solution of (3aR,6aR)-2-(4-(2,6-bis(benzyloxy)pyridin-3-yl)phenyl)octahydropyrrolo[3,4-c]pyrrole (3.5 g, 5.13 mmol) in MeCN (20 ml) at RT were added triethylamine (1.557 g, 15.39 mmol) and tert-butyl 2-chloroacetate (2.201 ml, 15.39 mmol). The reaction mixture was stirred at 60° C. for 2 h. Upon completion of reaction, as confirmed by LCMS, the reaction mixture was cooled to room temperature. Then water (100 mL) was added and extracted with EtOAc (2×250 mL), the combined organic layer was washed with water (200 mL), brine (100 mL), dried over sodium sulphate and concentrated under reduced pressure to get the crude product. The crude product was purified by flash chromatography using 30% of ethyl acetate/pet-ether as eluent to afford tert-butyl 2-((3aR,6aR)-5-(4-(2,6-bis(benzyloxy)pyridin-3-yl)phenyl)hexahydropyrrolo[3,4-c]pyrrol-2(1H)-yl)acetate (3.5 g, 70% yield) as off white solid. LCMS: C₃₇H₄₁N₃O₄ requires 591.3. found: m/z=592.2 [M+H]⁺.

### Step 4: Synthesis of tert-butyl 2-((3aR,6aR)-5-(4-(2,6-dioxopiperidin-3-yl)phenyl)hexahydropyrrolo[3,4-c]pyrrol-2(1H)-yl)acetate

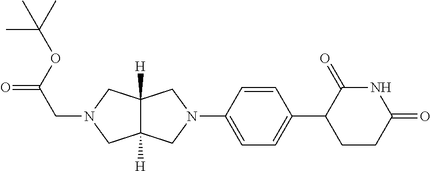 [SMILES: CC(C)(C)OC(=O)CN1C[C@@H]2CN(c3ccc(C4CCC(=O)NC4=O)cc3)C[C@H]2C1]

To a stirred solution of tert-butyl 2-((3aR,6aR)-5-(4-(2,6-bis(benzyloxy)pyridin-3-yl)phenyl)hexahydropyrrolo[3,4-c]pyrrol-2(1H)-yl)acetate (1.3 g, 2.197 mmol) in 1,4-dioxane (30 mL) was added Pd(OAc)₂ (0.200 g, 0.891 mmol) and 10% Pd/C (0.300 g, 2.82 mmol) under N₂ atmosphere at RT. The resulting mixture was stirred under H₂ (1 atm pressure) at RT for 16 h. Upon completion of reaction, as confirmed by LCMS, the reaction mixture was filtered through Celite bed washed with ethyl acetate (100 mL) and concentrated under reduced pressure. The crude product was washed with toluene (2×50 mL), MTBE (2×30 mL) and concentrated under reduced pressure to afford tert-butyl 2-((3aR,6aR)-5-(4-(2,6-dioxopiperidin-3-yl)phenyl)hexahydropyrrolo[3,4-c]pyrrol-2(1H)-yl)acetate (0.7 g, 73% yield) as off-white solid. LCMS: C₂₃H₃₁N₃O₄ requires 413.2, found: m/z=414.1 [M+H]⁺.

### Step 5: Synthesis of the Title Compound

To a stirred solution tert-butyl 2-((3aR,6aR)-5-(4-(2,6-dioxopiperidin-3-yl)phenyl)hexahydropyrrolo[3,4-c]pyrrol-2(1H)-yl)acetate (650 mg, 1.572 mmol) in DCM (6.5 mL) was cooled to 0° C. and TFA (1.203 ml, 15.72 mmol) was added. The reaction mixture was stirred at RT for 6 h. Upon completion of reaction, as confirmed by LCMS, n-hexane (50 mL) was added, stirred for 10 min. The clear solvent layer was decanted and repeated twice. The resulting residue was concentrated, dried under reduced pressure to get the crude product. The crude product was purified by RP-FC with MeCN in H₂O and the collected fraction lyophilized to afford the title compound (0.37 g, 64% yield, TFA salt) as off-white solid. LCMS: C₁₉H₂₃N₃O₄ requires 357.2, found: m/z=358.2 [M+H]⁺. ¹H NMR (400 MHz, DMSO-d₆) δ (ppm): 10.77-10.69 (m, 1H), 7.01 (d, J=8.5 Hz, 2H), 6.46 (d, J=8.8 Hz, 2H), 4.32 (s, 2H), 3.72-3.68 (m, 1H), 3.56-3.50 (m, 3H), 3.42 (br d, J=7.4 Hz, 3H), 3.10 (t, J=9.4 Hz, 2H), 2.68-2.58 (m, 2H), 2.50-2.48 (m, 2H), 2.18-2.08 (m, 1H), 2.03-1.94 (m, 1H).

### Intermediate 34

### 7-(4-(2,6-dioxopiperidin-3-yl)phenyl)-7-azaspiro[3.5]nonane-2-carbaldehyde

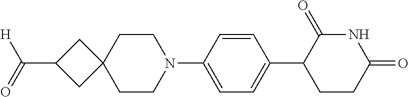 [SMILES: O=CC1CC2(CCN(c3ccc(C4CCC(=O)NC4=O)cc3)CC2)C1]

### Step 1: Synthesis of 7-(tert-butyl) 2-methyl 7-azaspiro[3.5]nonane-2,7-dicarboxylate

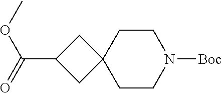 [SMILES: COC(=O)C1CC2(CCN(C(=O)OC(C)(C)C)CC2)C1]

To a stirred solution of 7-(tert-butoxycarbonyl)-7-azaspiro[3.5]nonane-2-carboxylic acid (4.5 g, 16.71 mmol) in DMF (40 mL) were added cesium carbonate (8.17 g, 25.06 mmol) followed by methyl iodide (2.85 g, 20.05 mmol). The mixture was stirred at RT for 16 h. Upon completion of reaction, as confirmed by LCMS, the reaction mixture was poured to ice cold water (100 mL) and extracted with ethyl acetate (2×200 mL). The organic layer was washed with brine solution (20 mL), dried over sodium sulphate and concentrated under reduced pressure to afford 7-(tert-butyl) 2-methyl 7-azaspiro[3.5]nonane-2,7-dicarboxylate (4 g, 80% yield) as yellow oil. LCMS: C₁₅H₂₅NO₄ requires 283.2. found: m/z=184.2 [M−Boc]⁺.

### Step 2: Synthesis of methyl 7-azaspiro[3.5]nonane-2-carboxylate

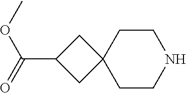

To a stirred solution of 7-(tert-butyl) 2-methyl 7-azaspiro[3.5]nonane-2,7-dicarboxylate (4 g, 14.12 mmol) in DCM (20 mL) was added 4M HCl in dioxane (35.3 mL, 141 mmol) at 0° C. The resulting mixture stirred at RT for 5 h. Upon completion of reaction, as confirmed by LCMS, the reaction mixture was concentrated under reduced pressure and washed with n-hexane (3×100 mL) and dried to afford methyl 7-azaspiro[3.5]nonane-2-carboxylate·HCl (3 g, 95%) as yellow solid. LCMS: C₁₀H₁₇NO₂ requires 183.2. found: m/z=184.2 [M+H]⁺.

### Step 3: Synthesis of methyl 7-(4-(2,6-bis(benzyloxy)pyridin-3-yl)phenyl)-7-azaspiro[3.5]nonane-2-carboxylate

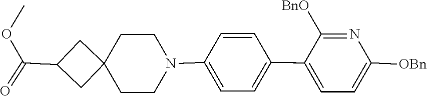 [SMILES: COC(=O)C1CC2(CCN(c3ccc(-c4ccc(OCc5ccccc5)nc4OCc4ccccc4)cc3)CC2)C1]

To a stirred solution of methyl 7-azaspiro[3.5]nonane-2-carboxylate·HCl (3.10 g, 14.11 mmol) and 2,6-bis(benzyloxy)-3-(4-bromophenyl)pyridine (6.3 g, 14.11 mmol) in dioxane (100 mL) was added, cesium carbonate (22.99 g, 70.6 mmol) at room temperature. The resulting mixture was purged with N₂ for 10 min. Then Pd₂(dba)₃ (0.646 g, 0.706 mmol) and RuPhos (0.659 g, 1.411 mmol) were added. The resulting reaction mixture was purged with N₂ for 5 min and heated the reaction mixture to 100° C. for 16 h. Upon completion of reaction, as confirmed by LCMS, the reaction mixture was cooled to RT and filtered through Celite pad and washed with ethyl acetate (300 mL). The filtrate was diluted with water (200 mL) and extracted with ethyl acetate (2×200 mL). The combined organic layer was dried over sodium sulphate and concentrated under vacuum to afford the crude product as brown liquid. The crude product was purified by flash chromatography using a gradient of 0-25% ethyl acetate/pet-ether to afford methyl 7-(4-(2,6-bis(benzyloxy)pyridin-3-yl)phenyl)spiro[3.5]nonane-2-carboxylate (5.3 g, 52% yield) as pale yellow solid. LCMS: C₃₅H₃₆N₂O₄ requires 548.3. found: m/z=549.2 [M+H]⁺.

### Step 4: Synthesis of (7-(4-(2,6-bis(benzyloxy)pyridin-3-yl)phenyl)-7-azaspiro[3.5]nonan-2-yl)methanol

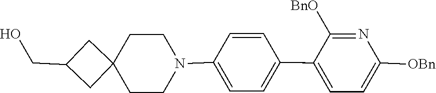 [SMILES: OCC1CC2(CCN(c3ccc(-c4ccc(OCc5ccccc5)nc4OCc4ccccc4)cc3)CC2)C1]

To a stirred solution of methyl 7-(4-(2,6-bis(benzyloxy)pyridin-3-yl)phenyl)spiro[3.5]nonane-2-carboxylate (5.3 g, 9.68 mmol) in THF (50 mL), LiAlH₄ (1 M soln. in THF, 9.68 mL, 19.35 mmol) was added dropwise at 0° C. The reaction mixture was stirred at RT for 2 h. Upon completion of reaction, as confirmed by LCMS, the mixture was quenched with ethyl acetate (25 mL), followed by saturated solution of sodium sulphate (20 mL). The reaction mixture was filtered through Celite pad, the filtrate was diluted with ethyl acetate (200 mL) and washed with water (2×100 mL), brine (50 mL), the organic layer was dried over sodium sulphate and concentrated under reduced pressure to get the crude product (5 g). The crude product was triturated with a mixture of DCM (50 mL) and hexane (40 mL) to afford (7-(4-(2,6-bis(benzyloxy)pyridin-3-yl)phenyl)spiro[3.5]nonan-2-yl)methanol (4.3 g, 80% yield) as green gum. LCMS: C₃₄H₃₆N₂O₃ requires 520.3. found: m/z=521.3 [M+H]⁺.

### Step 5: Synthesis of 3-(4-(2-(hydroxymethyl)-7-azaspiro[3.5]nonan-7-yl)phenyl)piperidine-2,6-dione

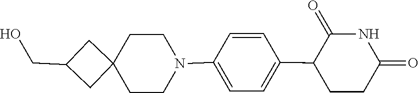 [SMILES: O=C1CCC(c2ccc(N3CCC4(CC3)CC(CO)C4)cc2)C(=O)N1]

To a stirred solution of (7-(4-(2,6-bis(benzyloxy)pyridin-3-yl)phenyl)spiro[3.5]nonan-2-yl)methanol (4.3 g, 8.27 mmol) in 1,4-dioxane (40 mL) were added Pd(OAc)₂ (0.4 g, 1.782 mmol) and 10% Pd/C (0.8 g, 7.52 mmol) under N₂ atmosphere at RT. The resulting reaction mixture stirred under H₂ pressure (1 atm) at RT for 16 h. Upon completion of reaction, as confirmed by LCMS, the reaction mixture was filtered through Celite bed washed with ethyl acetate (100 mL) and concentrated under reduced pressure. The crude was washed with toluene (2×50 mL), MTBE (2×30 mL) and concentrated under reduced pressure to afford 3-(4-(2-(hydroxymethyl)-7-azaspiro[3.5]nonan-7-yl)phenyl)piperidine-2,6-dione (2.5 g, 83% yield) as off-white solid. LCMS: C₂₀H₂₆N₂O₃ requires 342.2. found: m/z=343.2 [M+H]⁺.

### Step 6: Synthesis of the Title Compound

To a stirred solution of 3-(4-(2-(hydroxymethyl)-7-azaspiro[3.5]nonan-7-yl)phenyl)piperidine-2,6-dione (2.5 g, 7.30 mmol) in DMSO (5 mL) was added Dess-Martin periodinane (4.64 g, 10.95 mmol) under N₂ atmosphere at 0° C. The resulting reaction mixture was stirred at RT for 2 h. Upon completion of reaction, as confirmed by LCMS, the reaction mixture was quenched by sodium thiosulphate solution (20 mL) and extracted with ethyl acetate (2×50 mL). The organic layer was washed with sodium bicarbonate (20 mL), brine (20 mL), dried over sodium sulphate and concentrated under reduced pressure to get the crude product. The crude product was purified by RP-FC with MeCN in H₂O. The collected fraction was lyophilized to afford the title compound (0.903 g, 25% yield, as TFA·salt) as off-white solid. LCMS: C₂₀H₂₄N₂O₃ requires 340.2, found: m/z=341.3 [M+H]⁺. ¹H NMR (400 MHz, DMSO-d₆) δ (ppm): 10.82 (s, 1H), 9.75-9.66 (m, 1H), 7.33-7.04 (m, 4H), 3.81 (dd, J=4.0, 10.5 Hz, 1H), 3.30-3.22 (m, 3H), 3.19 (d, J=7.8 Hz, 2H), 2.71-2.62 (m, 1H), 2.46 (t, J=4.2 Hz, 1H), 2.28-2.07 (m, 2H), 2.02 (d, J=7.5 Hz, 4H), 1.83 (br s, 2H), 1.65 (br s, 2H).

### Intermediate 35

### Synthesis of 2-(1-(4-(2,6-dioxopiperidin-3-yl)phenyl)-4-methylpiperidin-4-yl)acetaldehyde

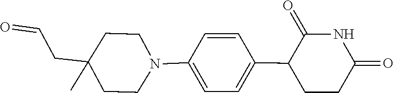 [SMILES: CC1(CC=O)CCN(c2ccc(C3CCC(=O)NC3=O)cc2)CC1]

### Step 1: Synthesis of 2-(4-methylpiperidin-4-yl)ethan-1-ol

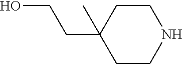

To a stirred solution of tert-butyl 4-(2-hydroxyethyl)-4-methylpiperidine-1-carboxylate (1 g, 4.11 mmol) in DCM (10 mL) was added 4M HCl in dioxane (0.027 mL, 4.11 mmol) at 0° C. The resulting mixture stirred at RT for 5 h. Upon completion of reaction, as confirmed by LCMS, the reaction mixture was concentrated under reduced pressure and washed with n-hexane (3×100 mL) and dried to afford 2-(4-methylpiperidin-4-yl)ethan-1-ol (HCl salt) as colorless gum. LCMS: C₈H₁₇NO requires 143.1. found: m/z=144.2 [M+H]⁺.

### Step 2: Synthesis of 2-(1-(4-(2,6-bis(benzyloxy)pyridin-3-yl)phenyl)-4-methylpiperidin-4-yl)ethan-1-ol

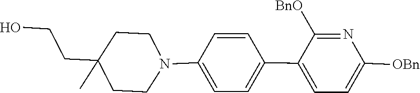 [SMILES: CC1(CCO)CCN(c2ccc(-c3ccc(OCc4ccccc4)nc3OCc3ccccc3)cc2)CC1]

To a stirred solution of 2,6-bis(benzyloxy)-3-(4-bromophenyl)pyridine (1.5 g, 3.36 mmol) and 2-(4-methylpiperidin-4-yl)ethan-1-ol HCl (0.604 g, 3.36 mmol) in 1,4 dioxane (50 mL) was added, cesium carbonate (5.47 g, 16.80 mmol) at room temperature. The resulting mixture was purged with N₂ for 10 min. Then Pd₂(dba)₃ (0.154 g, 0.168 mmol) and RuPhos (0.157 g, 0.336 mmol) were added. The resulting mixture was purged with N₂ for 5 min and heated the reaction mixture to 100° C. for 16 h. Upon completion of reaction, as confirmed by LCMS, the reaction mixture was cooled to RT and filtered through Celite pad and washed with ethyl acetate (300 mL).

The filtrate was diluted with water (200 mL) and extracted with ethyl acetate (2×200 mL). The combined organic layer was dried over sodium sulphate and concentrated under vacuum to afford crude compound as brown liquid. Similarly, an additional 1.5 g batch was performed to get 0.5 g of the crude product. The crude product from both batches were mixed and purified by flash chromatography with a gradient of 0-30% ethyl acetate/pet-ether to afford 2-(1-(4-(2,6-bis(benzyloxy)pyridin-3-yl)phenyl)-4-methylpiperidin-4-yl)ethan-1-ol (1 g, 30%) as pale yellow solid. LCMS: C₃₃H₃₆N₂O₃ requires 508.3. found: m/z=509.4 [M+H]⁺.

### Step 3: Synthesis of 3-(4-(4-(2-hydroxyethyl)-4-methylpiperidin-1-yl)phenyl)piperidine-2,6-dione

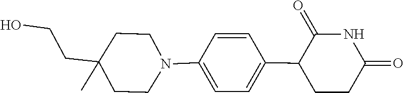 [SMILES: CC1(CCO)CCN(c2ccc(C3CCC(=O)NC3=O)cc2)CC1]

To a stirred solution of 2-(1-(4-(2,6-bis(benzyloxy)pyridin-3-yl)phenyl)-4-methylpiperidin-4-yl)ethan-1-ol (1 g, 1.966 mmol) in 1,4-dioxane (10 mL) were added Pd(OAc)₂ (0.1 g, 0.445 mmol) and 10% Pd/C (0.2 g, 1.879 mmol) under N₂ atmosphere at RT. The resulting mixture stirred under H₂ bladder pressure at RT for 16 h. Upon completion of reaction, as confirmed by LCMS, the reaction mixture was filtered through Celite bed washed with ethyl acetate (100 mL) and concentrated under reduced pressure. The crude was washed with toluene (2×50 mL), MTBE (2×30 mL) and concentrated under reduced pressure to afford 3-(4-(4-(2-hydroxyethyl)-4-methylpiperidin-1-yl)phenyl)piperidine-2,6-dione (0.5 g, 74%) as off-white solid. LCMS: C₁₉H₂₆N₂O₃ requires 330.2. found: m/z=331.2 [M+H]⁺.

### Step 4: Synthesis of the Title Compound

To a stirred solution of 3-(4-(4-(2-hydroxyethyl)-4-methylpiperidin-1-yl)phenyl)piperidine-2,6-dione (0.45 g, 1.362 mmol) in DMSO (5 mL) was added Dess-Martin periodinane (0.866 g, 2.043 mmol) under N₂ atmosphere at 0° C. The resulting reaction mixture was stirred at RT for 2 h. Upon completion of reaction, as confirmed by LCMS, the reaction mixture was quenched by sodium thiosulphate solution (20 mL) and extracted with ethyl acetate (2×50 mL). The organic layer was washed with sodium bicarbonate (20 mL), brine solution (20 mL), dried over sodium sulphate and concentrated under reduced pressure to get the crude product. The crude product was purified by RP-FC with MeCN in H₂O. The collected fraction was lyophilized to afford 2-(1-(4-(2,6-dioxopiperidin-3-yl)phenyl)-4-methylpiperidin-4-yl)acetaldehyde (0.11 g, 20% yield, TFA salt) as off-white solid. LCMS: C₁₉H₂₄N₂O₃ requires 328.2. found: m/z=329.2 [M+H]⁺. ¹H NMR (400 MHz, DMSO-d₆) δ (ppm): 10.89-10.79 (m, 1H), 9.86-9.73 (m, 1H), 7.38-6.81 (m, 4H), 3.73-3.62 (m, 1H), 3.41-3.31 (m, 2H), 3.28-3.17 (m, 2H), 2.70-2.63 (m, 1H), 2.50-2.45 (m, 3H), 2.25-1.99 (m, 2H), 1.82-1.69 (m, 2H), 1.67-1.56 (m, 2H), 1.14 (s, 3H)

### Intermediate 36

### 3-(4-(2-oxo-6-azaspiro[3.4]octan-6-yl)phenyl)piperidine-2,6-dione

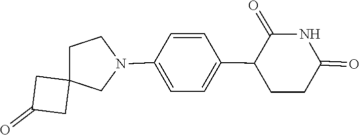 [SMILES: O=C1CC2(CCN(c3ccc(C4CCC(=O)NC4=O)cc3)C2)C1]

### Step 1: Synthesis of 6-azaspiro[3.4]octan-2-one

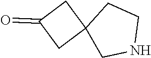

To a stirred solution of tert-butyl 2-oxo-6-azaspiro[3.4]octane-6-carboxylate (2.5 g, 11.10 mmol) in DCM (50 mL) was added 4M HCl in dioxane (27.7 mL, 111 mmol) at 0° C. The resulting reaction mixture was stirred at RT for 2 h. Upon completion of the reaction, as confirmed by LCMS, the reaction mixture was concentrated under reduced pressure and washed with n-hexane (3×100 mL) and dried to afford 6-azaspiro[3.4]octan-2-one (2.89 g, 94% yield, HCl salt) as brown gum. LCMS: C₇H₁₁NO requires 125.1. found: m/z=126.2 [M+H]⁺.

### Step 2: Synthesis of 6-(4-(2,6-bis(benzyloxy)pyridin-3-yl)phenyl)-6-azaspiro[3.4]octan-2-one

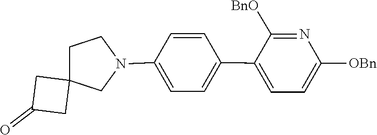 [SMILES: O=C1CC2(CCN(c3ccc(-c4ccc(OCc5ccccc5)nc4OCc4ccccc4)cc3)C2)C1]

To a stirred solution of 2,6-bis(benzyloxy)-3-(4-bromophenyl)pyridine (4.3 g, 9.641 mmol) and 6-azaspiro[3.4]octan-2-one HCl (1.55 g, 9.641 mmol) in 1,4 dioxane (50 mL) was added cesium carbonate (14.02 g, 43.0 mmol) at room temperature. The resulting mixture was purged with N₂ for 10 min. Then, Pd₂(dba)₃ (0.492 g, 0.538 mmol) and RuPhos (0.502 g, 1.075 mmol) were added. The resulting mixture was purged with N₂ for 5 min and heated to 100° C. for 16 h. Upon completion of reaction, as confirmed by LCMS, the reaction mixture was cooled to RT and filtered through Celite pad and washed with ethyl acetate (300 mL). The filtrate was diluted with water (200 mL) and extracted with ethyl acetate (2×200 mL). The combined organic layer was dried over sodium sulphate and concentrated under vacuum to afford crude compound as brown liquid. The crude compound was purified by flash chromatography with a gradient of 0-30% ethyl acetate/pet-ether to afford 6-(4-(2,6-bis(benzyloxy)pyridin-3-yl)phenyl)-6-azaspiro[3.4]octan-2-one (1 g, 75% purity) as pale yellow solid. LCMS: C₃₂H₃₀N₂O₃ requires 490.2. found: m/z=491.1 [M+H]⁺.

### Step 3: Synthesis of the Title Compound

To a stirred solution of 6-(4-(2,6-bis(benzyloxy)pyridin-3-yl)phenyl)-6-azaspiro[3.4]octan-2-one (1 g, 2.038 mmol) in 1,4-dioxane (10 mL) was added Pd(OAc)₂ (0.1 g, 0.445 mmol) and 10% Pd/C (0.2 g, 1.879 mmol)) under N₂ atmosphere at RT. The resulting reaction mixture was stirred under H₂ pressure (1 atm) at RT for 16 h. Upon completion of reaction, as confirmed by LCMS, the reaction mixture was filtered through Celite bed washed with ethyl acetate (100 mL) and concentrated under reduced pressure. The crude was washed with toluene (2×50 mL), MTBE (2×30 mL) and concentrated under reduced pressure The crude product was purified by RP-FC with MeCN in H₂O. The collected fraction was lyophilized to afford 3-(4-(2-oxo-6-azaspiro[3.4]octan-6-yl)phenyl)piperidine-2,6-dione (0.104 g, 11.4% yield, TFA salt) as off-white solid. LCMS: C₁₈H₂₀N₂O₃ requires 312.1. found: m/z=313.3 [M+H]⁺. ¹H NMR (400 MHz, DMSO-d₆) δ (ppm): 10.79-10.71 (m, 1H), 7.01 (d, J=8.6 Hz, 2H), 6.49 (d, J=8.8 Hz, 2H), 3.69 (d, J=5.8 Hz, 1H), 3.33 (t, J=6.8 Hz, 2H), 3.18-3.01 (m, 4H), 2.69-2.54 (m, 2H), 2.49-2.31 (m, 2H), 2.17 (t, J=6.7 Hz, 2H), 2.10-1.94 (m, 2H).

### Intermediate 37

### 8-(4-(2,6-dioxopiperidin-3-yl)phenyl)-2-oxa-8-azaspiro[4.5]decane-3-carbaldehyde

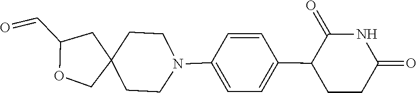 [SMILES: O=CC1CC2(CCN(c3ccc(C4CCC(=O)NC4=O)cc3)CC2)CO1]

### Step 1: Synthesis of tert-butyl 4-allyl-4-formylpiperidine-1-carboxylate

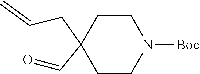 [SMILES: C=CCC1(C=O)CCN(C(=O)OC(C)(C)C)CC1]

To stirred solution of tert-butyl 4-formylpiperidine-1-carboxylate (5 g, 23.44 mmol) in THF (50 ml) at −25° C. was added 3-bromoprop-1-ene (2.431 ml, 28.1 mmol) and t-BuOK (3.16 g, 28.1 mmol) portion wise. The reaction mixture was stirred between −25° C. to −15° C. for 1 h. Upon completion of reaction, as confirmed by LCMS, the reaction mixture was cooled to RT. The reaction mixture was quenched by saturated ammonium chloride solution (200 mL) and extracted with ethyl acetate (2×200 mL). The organic layer was washed by brine (50 mL), dried over sodium sulphate, and concentrated to get the crude product. The crude compound was purified by flash chromatography with a gradient of 0-25% ethyl acetate/pet-ether to afford tert-butyl 4-allyl-4-formylpiperidine-1-carboxylate (3 g, 50% yield) as colorless oil. LCMS: C₁₄H₂₃NO₃ requires 253.2, found: m/z=153.1 [M−Boc]⁻.

### Step 2: Synthesis of tert-butyl 4-allyl-4-(hydroxymethyl)piperidine-1-carboxylate

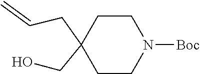 [SMILES: C=CCC1(CO)CCN(C(=O)OC(C)(C)C)CC1]

To stirred solution of tert-butyl 4-allyl-4-formylpiperidine-1-carboxylate (3 g, 11.84 mmol) in THF (30 mL) at 0° C. was added NaBH₄ (0.896 g, 23.68 mmol) portion wise. The reaction mixture was stirred at RT for 2 h. Upon completion of reaction, as confirmed by TLC, the reaction mixture was cooled to room temperature. Then water (100 mL) was added and extracted with EtOAc (2×250 mL), the combined organic layer was washed with brine (100 mL), dried over sodium sulphate and concentrated under reduced pressure to afford tert-butyl 4-allyl-4-(hydroxymethyl)piperidine-1-carboxylate (3 g, 90% yield) as colorless oil. LCMS: C₁₄H₂₅NO₃ requires 255.2. found: m/z=156.2 [M−Boc]⁻.

### Step 3: Synthesis of tert-butyl 3-(hydroxymethyl)-2-oxa-8-azaspiro[4.5]decane-8-carboxylate

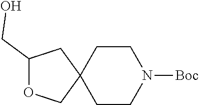 [SMILES: CC(C)(C)OC(=O)N1CCC2(CC1)COC(CO)C2]

To stirred solution of tert-butyl 4-allyl-4-(hydroxymethyl)piperidine-1-carboxylate (3 g, 11.75 mmol) in DCM (30 mL) was added m-CPBA (8.11 g, 47.0 mmol) at 0° C., the reaction mixture was stirred at RT for 16 h. Upon completion of reaction, as confirmed by TLC, the reaction mixture was cooled to room temperature. Then reaction mixture was quenched with sodium sulfite (100 mL), extracted with EtOAc (2×250 mL) and the combined organic layer was washed with sodium bicarbonate (100 mL), brine (100 mL), dried over sodium sulphate and concentrated under reduced pressure to get the crude product. The crude product was purified by flash chromatography with 20% of ethyl acetate in pet-ether to afford tert-butyl 3-(hydroxymethyl)-2-oxa-8-azaspiro[4.5]decane-8-carboxylate (2 g, 62% yield) as a colorless oil. LCMS: C₁₄H₂₅NO₄ requires 271.2. found: m/z=172.1 [M−Boc]⁻.

### Step 4: Synthesis of (2-oxa-8-azaspiro[4.5]decan-3-yl)methanol

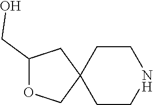 [SMILES: OCC1CC2(CCNCC2)CO1]

To a stirred solution of tert-butyl 3-(hydroxymethyl)-2-oxa-8-azaspiro[4.5]decane-8-carboxylate (2 g, 7.37 mmol) in DCM (20 mL) was added 4M HCl in dioxane (18.43 mL, 73.7 mmol) at 0° C. The resulting mixture stirred at RT for 2 h. Upon completion of reaction, as confirmed by LCMS, the reaction mixture was concentrated under reduced pressure and washed with n-hexane (3×100 mL), dried and lyophilized to afford (2-oxa-8-azaspiro[4.5]decan-3-yl)methanol (1.7 g, 97% yield, HCl salt) as off-white gummy solid. LCMS: C₉H₁₇NO₂ requires 171.1. found: m/z=172.2 [M+H]⁺.

### Step 5: Synthesis of (8-(4-(2,6-bis(benzyloxy)pyridin-3-yl)phenyl)-2-oxa-8-azaspiro[4.5]decan-3-yl)methanol

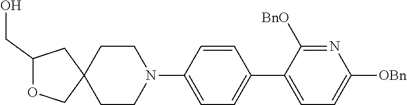 [SMILES: OCC1CC2(CCN(c3ccc(-c4ccc(OCc5ccccc5)nc4OCc4ccccc4)cc3)CC2)CO1]

To a stirred solution of (2-oxa-8-azaspiro[4.5]decan-3-yl)methanol HCl (0.931 g, 4.48 mmol) and 2,6-bis(benzyloxy)-3-(4-bromophenyl)pyridine (2 g, 4.48 mmol) in dioxane (30 mL) was added cesium carbonate (7.30 g, 22.40 mmol) at room temperature. The resulting mixture was purged with N₂ for 10 min. Then Pd₂(dba)₃ (0.041 g, 0.045 mmol) and RuPhos (0.042 g, 0.090 mmol) were added. The resulting reaction mixture was purged with N₂ for 5 min and heated the reaction mixture to 100° C. for 16 h. Upon completion of reaction, as confirmed by LCMS, the reaction mixture was cooled to RT and filtered through Celite pad and washed with ethyl acetate (300 mL). The filtrate was diluted with water (200 mL) and extracted with ethyl acetate (2×200 mL). The combined organic layer was dried over sodium sulphate and concentrated under vacuum to afford crude compound as brown liquid. The crude compound was purified by flash chromatography with a gradient of 0-30% ethyl acetate/pet-ether to afford (8-(4-(2,6-bis(benzyloxy)pyridin-3-yl)phenyl)-2-oxa-8-azaspiro[4.5]decan-3-yl)methanol (0.6 g, 50% yield) as pale yellow solid. LCMS: C₃₄H₃₆N₂O₄ requires 536.3. found: m/z=537.3 [M+H]⁺.

### Step 6: Synthesis of 3-(4-(3-(hydroxymethyl)-2-oxa-8-azaspiro[4.5]decan-8-yl)phenyl)piperidine-2,6-dione

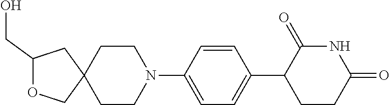 [SMILES: O=C1CCC(c2ccc(N3CCC4(CC3)COC(CO)C4)cc2)C(=O)N1]

To a stirred solution of (8-(4-(2,6-bis(benzyloxy)pyridin-3-yl)phenyl)-2-oxa-8-azaspiro[4.5]decan-3-yl)methanol (0.6 g, 1.118 mmol) in 1,4-dioxane (10 mL) were added Pd(OAc)₂ (0.05 g, 0.223 mmol) and 10% Pd/C (0.1 g, 0.940 mmol) under N₂ atmosphere at RT. The resulting mixture stirred under H₂ pressure (1 atm) at RT for 16 h. Upon completion of reaction, as confirmed by LCMS, the reaction mixture was filtered through Celite bed washed with ethyl acetate (100 mL) and concentrated under reduced pressure. The crude was washed with toluene (2×50 mL), MTBE (2×30 mL) and concentrated under reduced pressure to afford 3-(4-(3-(hydroxymethyl)-2-oxa-8-azaspiro[4.5]decan-8-yl)phenyl)piperidine-2,6-dione (0.4 g, 90% yield) as off-white solid. LCMS: C₂₀H₂₆N₂O₄ requires 358.2. found: m/z=359.1 [M+H]⁺.

### Step 7: Synthesis of the Title Compound

To a stirred solution of 3-(4-(3-(hydroxymethyl)-2-oxa-8-azaspiro[4.5]decan-8-yl)phenyl)piperidine-2,6-dione (0.4 g, 1.116 mmol) in DMSO (5 mL) was added Dess-Martin periodinane (0.710 g, 1.674 mmol) under N₂ atmosphere at 0° C. The resulting mixture was stirred at RT for 4 h. Upon completion of reaction, as confirmed by LCMS, the reaction mixture was quenched with sodium thiosulphate solution (20 mL) and extracted with ethyl acetate (2×50 mL). The organic layer was washed with sodium bicarbonate (20 mL), brine solution (20 mL) dried over sodium sulphate concentrated under reduced pressure. The crude product was purified by RP-FC with MeCN in H₂O. The collected fraction was lyophilized to afford 8-(4-(2,6-dioxopiperidin-3-yl)phenyl)-2-oxa-8-azaspiro[4.5]decane-3-carbaldehyde (0.124 g, TFA salt) as off-white solid. LCMS: C₂₀H₂₄N₂O₄ requires 356.2. found: m/z=357.2 [M+H]⁺. ¹H NMR (400 MHz, DMSO-d₆) δ (ppm): 10.88-10.80 (m, 1H), 9.64 (d, J=1.5 Hz, 1H), 7.24-6.98 (m, 4H), 4.43 (dd, J=1.4, 7.2 Hz, 1H), 3.84-3.69 (m, 2H), 3.30 (br d, J=6.3 Hz, 2H), 2.73-2.52 (m, 4H), 2.46 (br t, J=3.8 Hz, 2H), 2.37-1.94 (m, 4H), 1.92-1.77 (m, 2H), 1.75-1.57 (m, 4H).

### Intermediate 38

### rac-(4-{4-[(3R)-2,6-dioxopiperidin-3-yl]phenyl}piperazin-1-yl)acetic acid

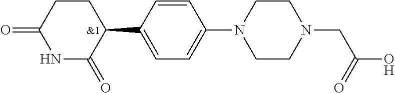 [SMILES: O=C(O)CN1CCN(c2ccc([C@@H]3CCC(=O)NC3=O)cc2)CC1]

Step-1: Synthesis of tert-butyl 2-[4-(4-bromophenyl)piperazin-1-yl]acetate

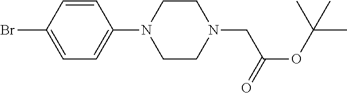 [SMILES: CC(C)(C)OC(=O)CN1CCN(c2ccc(Br)cc2)CC1]

To a mixture of tert-butyl 2-(piperazin-1-yl)acetate (500 mg, 2.496 mmol, 1 equiv) in toluene (5 mL) was added dibromobenzene (588 mg, 2.496 mmol, 1 equiv), Pd₂(dba)₃ (228 mg, 0.250 mmol, 0.1 equiv), BINAP (310 mg, 0.499 mmol, 0.2 equiv) and Cs₂CO₃ (2440 mg, 7.488 mmol, 3 equiv) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1/1) to afford tert-butyl 2-[4-(4-bromophenyl)piperazin-1-yl]acetate (300 mg, 33.82%) as a yellow oil. LCMS: (C₁₆H₂₃BrN₂O₂) desired mass=355.1; observed mass=355.1 [M+H]⁺.

### Step-2: Synthesis of tert-butyl 2-(4-{4-[2,6-bis(benzyloxy)pyridin-3-yl]phenyl}piperazin-1-yl)acetate

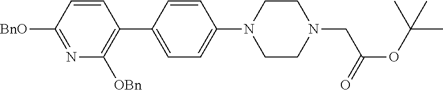 [SMILES: CC(C)(C)OC(=O)CN1CCN(c2ccc(-c3ccc(OCc4ccccc4)nc3OCc3ccccc3)cc2)CC1]

To a mixture of tert-butyl 2-[4-(4-bromophenyl)piperazin-1-yl]acetate (1 g, 2.815 mmol, 1 equiv) in dioxane (50 mL) was added 2,6-bis(benzyloxy)-3-(4,4,5,5-tetramethyl-1,3,2-dioxaborolan-2-yl)pyridine (1.76 g, 4.223 mmol, 1.5 equiv), XPhos Pd G2 (0.44 g, 0.563 mmol, 0.2 equiv), K₃PO₄ (1.79 g, 8.445 mmol, 3 equiv) in H₂O (10 mL). The resulting mixture was stirred for 2 h at 60° C. under nitrogen atmosphere. The resulting mixture was concentrated under reduced pressure. The residue was purified by silica gel column chromatography, eluted with PE/EA (1/2) to afford tert-butyl 2-(4-{4-[2,6-bis(benzyloxy)pyridin-3-yl]phenyl}piperazin-1-yl)acetate (650 mg, 36.74%) as a yellow solid. LCMS: (C₃₅H₃₉N₃O₄) desired mass=566.3; observed mass=566.3 [M+H]⁺.

### Step-3: Synthesis of tert-butyl 2-{4-[4-(2,6-dioxopiperidin-3-yl)phenyl]piperazin-1-yl}acetate

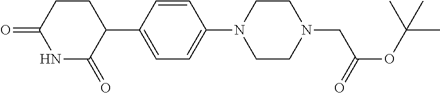 [SMILES: CC(C)(C)OC(=O)CN1CCN(c2ccc(C3CCC(=O)NC3=O)cc2)CC1]

To mixture of tert-butyl 2-(4-{4-[2,6-bis(benzyloxy)pyridin-3-yl]phenyl}piperazin-1-yl)acetate (650 mg, 1.149 mmol, 1 equiv) in THF (25 mL) was added Pd/C (650 mg) at room temperature. The resulting mixture was stirred overnight at room temperature under hydrogen atmosphere. The resulting mixture was filtered. The filtrate was concentrated under reduced pressure. This resulted in tert-butyl 2-{4-[4-(2,6-dioxopiperidin-3-yl)phenyl]piperazin-1-yl}acetate (400 mg, crude) as a light yellow solid. LCMS: (C₂₁H₂₉N₃O₄) desired mass=388.2; observed mass=388.2 [M+H]⁺.

### Step 4: Synthesis of rac-(4-{4-[(3R)-2,6-dioxopiperidin-3-yl]phenyl}piperazin-1-yl)acetic acid

A mixture of tert-butyl 2-{4-[4-(2,6-dioxopiperidin-3-yl)phenyl]piperazin-1-yl}acetate (2 g, 5.162 mmol, 1 equiv) in HCl (gas)/1,4-dioxane (20 mL, 4M) was stirred for 2 h at room temperature. The resulting mixture was concentrated under vacuum and further lyophilized. The crude product was purified by trituration with n-hexane. This result in rac-(4-{4-[(3R)-2,6-dioxopiperidin-3-yl]phenyl}piperazin-1-yl)acetic acid (1.5316 g, 81.58%) as an off-white solid. LCMS: (C₁₇H₂₁N₃O₄) desired mass=332.2; observed mass=332.2 [M+H]⁺.

¹H NMR (400 MHz, DMSO-d₆) δ 10.79 (s, 1H), 7.12 (d, J=8.0 Hz, 2H), 6.97 (d, J=8.4 Hz, 2H), 4.22 (s, 2H), 4.07-2.88 (m, 9H), 2.72-2.59 (m, 1H), 2.48-2.40 (m, 1H), 2.22-2.06 (m, 1H), 2.06-1.94 (m, 1H).

### Intermediate 39

### 3-(4-(3-oxo-1-oxa-8-azaspiro[4.5]decan-8-yl)phenyl)piperidine-2,6-dione

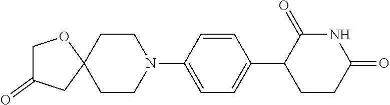 [SMILES: O=C1COC2(CCN(c3ccc(C4CCC(=O)NC4=O)cc3)CC2)C1]

### Step 1: Synthesis of 8-(4-(2,6-bis(benzyloxy)pyridin-3-yl)phenyl)-1-oxa-8-azaspiro[4.5]decan-3-one

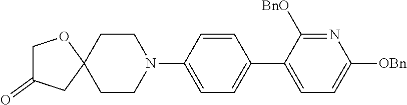 [SMILES: O=C1COC2(CCN(c3ccc(-c4ccc(OCc5ccccc5)nc4OCc4ccccc4)cc3)CC2)C1]

To a stirred solution of 2,6-bis(benzyloxy)-3-(4-bromophenyl)pyridine 1 (5 g, 11.2 mmol), 1-oxa-8-azaspiro[4.5]decan-3-one·HCl 2 (2.141 g, 11.2 mmol) in dioxane (60 mL) was added cesium carbonate (18.2 g, 56 mmol) at room temperature. The resulting mixture was purged with N₂ for 10 min. Then Pd2(dba)3 (0.513 g, 0.560 mmol) and RuPhos (0.523 g, 1.120 mmol) were added. The resulting mixture was purged with N₂ for 5 min and heated the reaction mixture to 100° C. for 16 h. Upon completion of reaction, as confirmed by LCMS, the reaction mixture was cooled to RT and filtered through Celite pad and washed with ethyl acetate (300 mL). The filtrate was diluted with water (200 mL) and extracted with ethyl acetate (2×200 mL). The combined organic layer was dried over sodium sulphate and concentrated under vacuum to afford the crude compound as brown liquid. The crude compound was purified by Biotage-Isolera (silica-gel: 230-400 mesh) using ethyl acetate/pet-ether (0-30%) as eluent to get the crude product (2 g) as yellow solid. The crude product was purified by prep-HPLC purification using—Column: Xselect C18 250 mm, Method: 0.1% TFA in water/MeCN, Flow rate: 12 mL/min. The collected fraction was lyophilized to afford 8-(4-(2,6-bis(benzyloxy)pyridin-3-yl)phenyl)-1-oxa-8-azaspiro[4.5]decan-3-one (1.1 g 13% yield) as pale yellow solid: LCMS: m/z=522.1 [M+H]⁺.

1H NMR (400 MHz, DMSO-d6) δ (ppm): 7.75-7.69 (m, 1H), 7.51-7.30 (m, 12H), 7.14-7.03 (m, 2H), 6.54-6.47 (m, 1H), 5.41 (s, 2H), 5.37 (s, 2H), 4.03 (s, 2H), 3.43-3.27 (m, 4H), 2.51 (s, 2H), 1.86 (d, J=4.5 Hz, 4H).

### Step 2: Synthesis of Title Compound

To a stirred solution of 8-(4-(2,6-bis(benzyloxy)pyridin-3-yl)phenyl)-1-oxa-8-azaspiro[4.5]decan-3-one 3 (1.1 g, 2.113 mmol in 1,4-dioxane (30 mL) was added Pd(OAc)₂ (0.150 g, 0.668 mmol) and 10% Pd/C (0.200 g, 1.879 mmol) under N₂ atmosphere at RT. The resulting mixture was stirred under H₂ pressure (1 atm) at RT for 16 h. Upon completion of the reaction, as confirmed by LCMS, the reaction mixture was filtered through Celite bed washed with ethyl acetate (100 mL) and concentrated under reduced pressure. The crude compound was washed with toluene (2×50 mL), MTBE (2×30 mL), the resulting residue was concentrated under reduced pressure and lyophilized to afford the title compound (0.504 g, 52% yield, TFA salt) as off-white solid. LCMS: m/z=343.2 [M+H]⁺.

¹H NMR (400 MHz, DMSO-d6) δ (ppm): 10.85-10.79 (m, 1H), 7.10 (br s, 2H), 7.03-7.01 (m, 2H), 4.07-4.06 (m, 2H), 3.76 (br s, 1H), 3.28 (br s, 4H), 2.64 (d, J=5.5 Hz, 2H), 2.11-2.04 (m, 4H), 2.51 (s, 2H), 1.85-1.84 (m, 4H).

### Intermediate 40

### rac-(R)-1-(4-(2,6-dioxopiperidin-3-yl)-2,3-difluorophenyl)piperidine-4-carbaldehyde

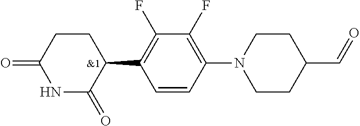 [SMILES: O=CC1CCN(c2ccc([C@@H]3CCC(=O)NC3=O)c(F)c2F)CC1]

### Step 1: Synthesis of 1-(4-bromo-2,3-difluorophenyl)-4-(dimethoxymethyl)piperidine

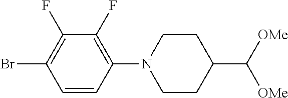 [SMILES: COC(OC)C1CCN(c2ccc(Br)c(F)c2F)CC1]

To a solution of 1-bromo-2,3-difluoro-4-iodobenzene (20.0 g, 62.7 mmol, 1.00 eq), 4-(dimethoxymethyl)piperidine (10.9 g, 68.9 mmol, 1.10 eq), BINAP (1.56 g, 2.51 mmol, 0.04 eq), t-BuONa (12.0 g, 125 mmol, 2.00 eq) and Pd₂(dba)₃ (1.15 g, 1.25 mmol, 0.02 eq) was added in toluene (200 mL) at 20° C. and purged with N₂ for 3 times. The mixture was stirred at 100° C. for 16 hrs. LCMS showed 28.9% of the desired MS was detected. The mixture was concentrated directly by vacuum. The residue was purified by column chromatography (SiO₂, Ethyl acetate/Petroleum ether=0/1 to 1/20, Ethyl acetate/Petroleum ether=1/10, Rf=0.30). Then the fraction was concentrated under vacuum to give the title compound (14.0 g, 31.5 mmol, 50.3% yield, 79.0% purity) was obtained as a yellow solid. δ 7.21-7.12 (m, 1H), 6.67-6.55 (m, 1H), 4.10 (d, J=7.1 Hz, 1H), 3.46 (br d, J=12.0 Hz, 2H), 3.38 (s, 6H), 2.73-2.60 (td, 2H), 1.85 (br d, J=13.9 Hz, 2H), 1.56-1.43 (m, 2H), 1.26 (t, 1H)

### Step 2: Synthesis of 2,6-bis(benzyloxy)-3-(4-(4-(dimethoxymethyl)piperidin-1-yl)-2,3-difluorophenyl)pyridine

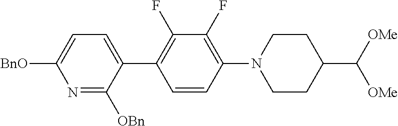 [SMILES: COC(OC)C1CCN(c2ccc(-c3ccc(OCc4ccccc4)nc3OCc3ccccc3)c(F)c2F)CC1]

To a solution of 1-(4-bromo-2,3-difluorophenyl)-4-(dimethoxymethyl)piperidine (13.0 g, 29.7 mmol, 1.00 eq), 2,6-bis(benzyloxy)-3-(4,4,5,5-tetramethyl-1,3,2-dioxaborolan-2-yl)pyridine (16.1 g, 38.6 mmol, 1.30 eq), Cs₂CO₃ (24.1 g, 74.2 mmol, 2.50 eq) and Pd(dppf)Cl₂·CH₂Cl₂ (1.21 g, 1.48 mmol, 0.05 eq) was added in dioxane (260 mL) and H₂O (52.0 mL) at 20° C. and purged with N₂ for 3 times. The mixture was stirred at 100° C. for 16 hrs under N₂ atmosphere. The residue was added to water (200 mL) and extracted with ethyl acetate (60.0 mL×3). The combined organic layers were washed with brine (100 mL×1), dried over Na₂SO₄, filtered and concentrated. The residue was purified by column chromatography (SiO₂, Ethyl acetate/Petroleum ether=0/1 to 1/20, Ethyl acetate/Petroleum ether=1/10, Rf=0.25). Then the fraction was concentrated under vacuum.

The desired product 2,6-bis(benzyloxy)-3-(4-(4-(dimethoxymethyl)piperidin-1-yl)-2,3-difluorophenyl)pyridine (8.80 g, 13.8 mmol, 46.5% yield, 88.0% purity) was obtained as a yellow solid. NMR: (400 MHz, CDCl₃) δ 7.56-7.29 (m, 11H), 7.10-6.95 (m, 1H), 6.46 (d, J=8.1 Hz, 1H), 5.46-5.23 (m, 4H), 4.12 (d, J=7.2 Hz, 1H), 3.54 (br d, J=11.9 Hz, 2H), 3.40 (s, 6H), 2.71 (t, J=11.4 Hz, 2H), 1.87 (br d, J=12.8 Hz, 2H), 1.81-1.71 (m, 1H), 1.64-1.48 (m, 3H)

### Step 3: Synthesis of rac-(R)-3-(4-(4-(dimethoxymethyl)piperidin-1-yl)-2,3-difluorophenyl)piperidine-2,6-dione

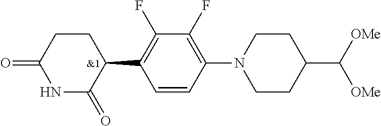 [SMILES: COC(OC)C1CCN(c2ccc([C@@H]3CCC(=O)NC3=O)c(F)c2F)CC1]

To a solution of rac-(R)-3-(4-(4-(dimethoxymethyl)piperidin-1-yl)-2,3-difluorophenyl)piperidine-2,6-dione (43.0 g, 76.7 mmol, 1.00 eq) in THF (430 mL) was added Pd/C (12.9 g, 12.1 mmol, 10% purity) and Pd(OH)₂ (12.9 g, 27.5 mmol, 30% purity) under N₂ atmosphere. The suspension was degassed and purged with H₂ for 3 times. The mixture was stirred under H₂ (50 psi) at 25° C. for 6 hrs at which time the mixture was filtered and concentrated. The crude was triturated by MTBE (20.0 mL) at 25° C. for 30 mins, and filtered and the cake was dried under reduced pressure to give rac-(R)-3-(4-(4-(dimethoxymethyl)piperidin-1-yl)-2,3-difluorophenyl)piperidine-2,6-dione (8.00 g, 20.9 mmol, 88.8% yield) was obtained as a white solid.

¹H NMR: (400 MHz, DMSO-d₆) δ 10.92 (s, 1H), 7.18-6.69 (m, 2H), 4.16 (d, J=6.5 Hz, 1H), 4.11-4.00 (m, 1H), 3.47-3.38 (m, 3H), 3.32 (s, 6H), 2.85-2.64 (m, 3H), 2.30-2.13 (m, 1H), 2.10-1.95 (m, 1H), 1.84-1.68 (m, 3H), 1.52-1.35 (m, 2H)

### Step 4: Synthesis of Title Compound

To a solution of rac-(R)-3-(4-(4-(dimethoxymethyl)piperidin-1-yl)-2,3-difluorophenyl)piperidine-2,6-dione (4.00 g, 10.4 mmol, 1.00 eq) in THF (93.0 mL) was added dropwise slowly in HCl (2.00 M, 93.1 mL, 17.8 eq). After addition, the reaction solution was stirred at 70° C. for 1 hr. To the reaction mixture was then added saturated NaHCO₃ solution until pH=7, then extracted with EtOAc (30.0 mL×3), dried over Na₂SO₄, filtered and concentrated to get the crude. The crude was triturated with EtOAc (10.0 mL) at 25° C. for 30 mins to give the title compound (2.00 g, 5.86 mmol, 56.0% yield, 98.4% purity) as a white solid. LCMS: m/z=337.2 (M+H)+¹H NMR: (400 MHz, DMSO-d₆) δ 10.88 (s, 1H), 9.64 (s, 1H), 6.99 (br t, J=7.5 Hz, 1H), 6.82 (br t, J=7.9 Hz, 1H), 4.12-3.94 (m, 1H), 3.35-3.26 (m, 2H), 2.88-2.67 (m, 3H), 2.55 (br d, J=3.1 Hz, 1H), 2.49-2.43 (m, 1H), 2.26-2.09 (m, 1H), 2.05-1.88 (m, 3H), 1.73-1.56 (m, 2H)

### Intermediate 41

### Synthesis of rac-(R)-2-(1-(4-(2,6-dioxopiperidin-3-yl)phenyl)-4-fluoropiperidin-4-yl)acetaldehyde

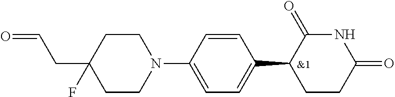 [SMILES: O=CCC1(F)CCN(c2ccc([C@@H]3CCC(=O)NC3=O)cc2)CC1]

### Step 1: Synthesis of tert-butyl 4-fluoro-4-(2-hydroxyethyl)piperidine-1-carboxylate

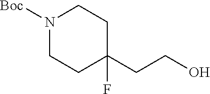 [SMILES: CC(C)(C)OC(=O)N1CCC(F)(CCO)CC1]

A mixture of tert-butyl 4-(2-ethoxy-2-oxoethyl)-4-fluoropiperidine-1-carboxylate (10.5 g, 36.3 mmol, 1.00 eq) in THF (105 mL) was degassed under vacuum and purged with N₂ several times. The mixture was cooled to −20˜0° C., then LiAlH₄ (2.50 M, 29.0 mL, 2.00 eq) was added to the mixture dropwise at −20˜0° C. under N₂. The resulting mixture was stirred at −20˜0° C. for 2 hrs under N₂. H₂O (5.00 mL) was added to the reaction mixture dropwise at −20˜0° C. to quench the reaction under N₂ flow, then the mixture was dried with Na₂SO₄, filtered and concentrated under vacuum. The crude product tert-butyl 4-fluoro-4-(2-hydroxyethyl)piperidine-1-carboxylate (9.00 g, crude) as a colorless oil was used into the next step without further purification. ¹H NMR (400 MHz, CDCl₃) δ 4.00-3.79 (m, 4H), 3.08 (br t, J=12.0 Hz, 2H), 1.95-1.83 (m, 5H), 1.70-1.50 (m, 2H), 1.45 (s, 9H)

### Step 2: Synthesis of 2-(4-fluoropiperidin-4-yl)ethan-1-ol hydrochloride

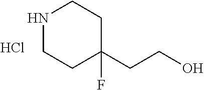

To mixture of tert-butyl 4-fluoro-4-(2-hydroxyethyl)piperidine-1-carboxylate (10.6 g, 43.1 mmol, 1.00 eq) in dioxane (11.0 mL) was added HCl/dioxane (2.00 M, 53.2 mL, 2.47 eq) at 10-20° C. The resulting mixture was stirred at 10-20° C. for 12 hrs. The mixture was concentrated under vacuum. The crude 2-(4-fluoropiperidin-4-yl)ethan-1-ol hydrochloride (7.92 g, 43.1 mmol, 100% yield, HCl) as a white solid was used into the next step without further purification. ¹H NMR (400 MHz, DMSO) δ 9.20 (br s, 2H), 4.74-4.01 (m, 1H), 3.61-3.49 (m, 2H), 3.16 (br d, J=12.4 Hz, 2H), 3.02-2.85 (m, 2H), 2.10-1.87 (m, 4H), 1.86-1.71 (m, 2H)

### Step 3: Synthesis of 2-(1-(4-bromophenyl)-4-fluoropiperidin-4-yl)ethan-1-ol

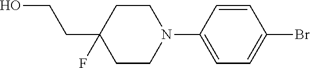 [SMILES: OCCC1(F)CCN(c2ccc(Br)cc2)CC1]

To a mixture of 1-bromo-4-iodobenzene (11.7 g, 41.4 mmol, 1.10 eq), 2-(4-fluoropiperidin-4-yl)ethan-1-ol hydrochloride (6.92 g, 37.7 mmol, 1.00 eq, HCl) in DMSO (82.0 mL) was added K₂CO₃ (15.6 g, 113 mmol, 3.00 eq), CuI (1.44 g, 7.54 mmol, 0.20 eq), L-PROLINE (1.74 g, 15.0 mmol, 0.40 eq) under N₂ at 15-25° C. The resulting mixture was stirred at 75-80° C. for 16 hrs under N₂. The mixture was poured into H₂O (200 mL), extracted with ethyl acetate (80.0 mL*3), the combined organic layers were washed with 3.00% NH₃—H₂O solution (50.0 mL), brine (50.0 mL), dried with Na₂SO₄, filtered and concentrated in vacuum. The residue was purified by column chromatography (SiO₂, Petroleum ether/Ethyl acetate=10/1 to 3/1, Petroleum ether/Ethyl acetate=2/1, R_{f}=0.21), concentrated in vacuum. The crude compound 2-(1-(4-bromophenyl)-4-fluoropiperidin-4-yl)ethan-1-ol (5.70 g, 18.2 mmol, 48.4% yield, 96.7% purity) was obtained as a yellow solid. LCMS: C₁₃H₁₇BrFNO requires: 301.0. found: m/z=304.1 (M+H)+.

### Step 4: Synthesis of 2-(1-(4-(2,6-bis(benzyloxy)pyridin-3-yl)phenyl)-4-fluoropiperidin-4-yl)ethan-1-ol

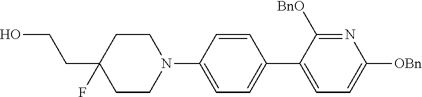 [SMILES: OCCC1(F)CCN(c2ccc(-c3ccc(OCc4ccccc4)nc3OCc3ccccc3)cc2)CC1]

To a mixture of 2-(1-(4-bromophenyl)-4-fluoropiperidin-4-yl)ethan-1-ol (5.70 g, 18.8 mmol, 1.00 eq) in dioxane (114 mL) and H₂O (22.8 mL) was added 2,6-bis(benzyloxy)-3-(4,4,5,5-tetramethyl-1,3,2-dioxaborolan-2-yl)pyridine (10.2 g, 24.5 mmol, 1.30 eq), Cs₂CO₃ (15.4 g, 47.2 mmol, 2.50 eq), the suspension was degassed under vacuum and purged with N₂ several times. Then Pd(dppf)Cl₂·CH₂Cl₂ (1.54 g, 1.89 mmol, 0.10 eq) was added to the mixture under N₂. The resulting mixture was stirred at 100° C. for 16 hrs. The reaction mixture was poured into water (400 mL) and extracted with ethyl acetate (200 mL*3), the organic phase was washed with brine (200 mL), dried over Na₂SO₄, concentrated under vacuum. The residue was purified by column chromatography (SiO₂, Petroleum ether/Ethyl acetate=10/1 to 3/1, Petroleum ether/Ethyl acetate=2/1, R_{f}=0.21), concentrated in vacuum. 2-(1-(4-(2,6-bis(benzyloxy)pyridin-3-yl)phenyl)-4-fluoropiperidin-4-yl)ethan-1-ol (7.10 g, 13.7 mmol, 72.8% yield, 99.1% purity) was obtained as a yellow solid. LCMS: C₃₂H₃₃FN₂O₃ requires: 512.2. found: m/z=513.3 (M+H)+¹H NMR (400 MHz, CDCl₃) δ 7.60 (d, J=8.0 Hz, 1H), 7.51 (d, J=8.8 Hz, 2H), 7.48-7.28 (m, 10H), 6.99 (br d, J=8.4 Hz, 2H), 6.47 (d, J=8.2 Hz, 1H), 5.52-5.31 (m, 4H), 3.98-3.83 (m, 2H), 3.55 (br d, J=12.4 Hz, 2H), 3.15 (dt, J=2.0, 12.2 Hz, 2H), 2.09-1.78 (m, 6H), 1.65 (br d, J=4.4 Hz, 1H)

### Step 5: Synthesis of rac-(R)-3-(4-(4-fluoro-4-(2-hydroxyethyl)piperidin-1-yl)phenyl)piperidine-2,6-dione

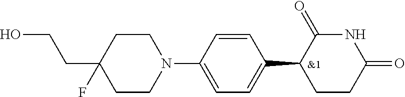 [SMILES: O=C1CC[C@@H](c2ccc(N3CCC(F)(CCO)CC3)cc2)C(=O)N1]

To a mixture of 2-(1-(4-(2,6-bis(benzyloxy)pyridin-3-yl)phenyl)-4-fluoropiperidin-4-yl)ethan-1-ol (6.60 g, 12.9 mmol, 1.00 eq) in THF (200 mL) was added Pd/C (1.98 g, 1.86 mmol, 10% purity, 0.145 eq), then Pd(OH)₂ (1.98 g, 2.82 mmol, 20% purity, 0.219 eq) under N₂. The suspension was degassed and purged with H₂ for 3 times. The mixture was stirred under H₂ (50 Psi) at 25° C. for 14 hrs. The mixture was filtered through a pad of celite to get the filtrate. The filter cake was washed with THF (200 mL*3), the combined filtrate was concentrated in vacuum. The residue was triturated with MTBE (30.0 mL) at 20-25° C. for 30 mins, filtered, the filter cake was washed with MTBE (8.00 mL*3), dried in vacuum. rac-(R)-3-(4-(4-fluoro-4-(2-hydroxyethyl)piperidin-1-yl)phenyl)piperidine-2,6-dione (3.93 g, 11.0 mmol, 85.6% yield, 93.8% purity) was obtained as a white solid. LCMS: C₁₈H₂₃FN₂O₃ requires: 334.2. found: m/z=335.2 (M+H)^{+ 1}H NMR (400 MHz, DMSO) δ 10.77 (s, 1H), 7.04 (br d, J=8.4 Hz, 2H), 6.92 (br d, J=8.4 Hz, 2H), 4.48 (br t, J=5.2 Hz, 1H), 3.72 (br dd, J=4.8, 10.8 Hz, 1H), 3.63-3.52 (m, 2H), 3.46 (br d, J=12.0 Hz, 2H), 2.92 (br t, J=12.0 Hz, 2H), 2.71-2.56 (m, 1H), 2.48-2.34 (m, 1H), 2.22-1.94 (m, 2H), 1.92-1.65 (m, 6H)

### Step 6: Synthesis of Title Compound

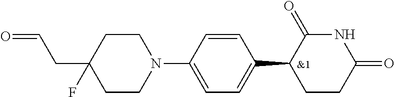 [SMILES: O=CCC1(F)CCN(c2ccc([C@@H]3CCC(=O)NC3=O)cc2)CC1]

To a mixture of rac-(R)-3-(4-(4-fluoro-4-(2-hydroxyethyl)piperidin-1-yl)phenyl)piperidine-2,6-dione (3.40 g, 10.17 mmol, 1.00 eq) in DMSO (34.0 mL) was added IBX (4.27 g, 15.2 mmol, 1.50 eq) at 20-25° C. and the resulting mixture was stirred at 20-25° C. for 4 hrs. The mixture was poured into H₂O (150 mL), extracted with ethyl acetate (100 mL*4), the combined organic layers were washed with sat.NaHCO₃ (50.0 mL), brine (150 mL), dried with Na₂SO₄, filtered and concentrated in vacuum. The crude product was triturated with ethyl acetate (50.0 mL) at 25° C. for 30 mins, filtered, the filter cake was washed with ethyl acetate (5.00 mL*3), dried in vacuum. rac-(R)-2-(1-(4-(2,6-dioxopiperidin-3-yl)phenyl)-4-fluoropiperidin-4-yl)acetaldehyde (2.65 g, 7.75 mmol, 76.2% yield, 97.2% purity) was obtained as a yellow solid. LCMS: C₁₈H₂₁FN₂O₃ requires: 332.2. found: m/z=333.2 (M+H)+.

¹H NMR (400 MHz, DMSO) δ 10.77 (s, 1H), 9.79 (br d, J=1.2 Hz, 1H), 7.06 (d, J=8.8 Hz, 2H), 6.93 (d, J=8.8 Hz, 2H), 3.73 (dd, J=4.8, 11.0 Hz, 1H), 3.58-3.45 (m, 2H), 3.08-2.91 (m, 2H), 2.87-2.75 (m, 2H), 2.69-2.57 (m, 1H), 2.49-2.41 (m, 1H), 2.20-2.06 (m, 1H), 2.05-1.80 (m, 5H)

### Intermediate 42

### rel-(R)-1-(4-(2,6-dioxopiperidin-3-yl)phenyl)piperidine-4-carboxylic acid

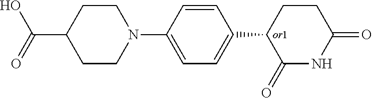 [SMILES: O=C1CC[C@@H](c2ccc(N3CCC(C(=O)O)CC3)cc2)C(=O)N1]

### Step1: Synthesis of tert-butyl 1-(4-(2,6-bis(benzyloxy)pyridin-3-yl)phenyl)piperidine-4-carboxylate

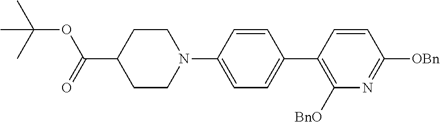 [SMILES: CC(C)(C)OC(=O)C1CCN(c2ccc(-c3ccc(OCc4ccccc4)nc3OCc3ccccc3)cc2)CC1]

A mixture of 2,6-bis(benzyloxy)-3-(4-bromophenyl)pyridine (170 g, 380 mmol), tert-butyl piperidine-4-carboxylate (84.6 g, 457 mmol), K₃PO₄ (242 g, 1.14 mol) and XPhos Pd G₃ (32.2 g, 38.0 mmol) in DMF (1000 mL) was stirred at 100° C. until complete as judged by LCMS. Once complete, to the mixture was added H₂O (2.00 L) and extracted with ethyl acetate (700 mL×2). The organic layers were washed with brine (700 mL×2), dried over anhydrous Na₂SO₄, filtered, and concentrated to give a crude residue. The residue was purified by column chromatography (Petroleum ether in Ethyl acetate=100:1 to 20:1) to give the desired compound as a yellow solid (160 g, mmol, 71% yield). LCMS C₃₅H₃₈N₂O₄ requires 550.3, found m/z=551.3 [M+H]⁺.

### Step 2: Synthesis of rac-tert-butyl (R)-1-(4-(2,6-dioxopiperidin-3-yl)phenyl)piperidine-4-carboxylate

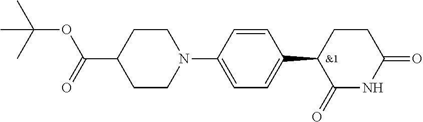 [SMILES: CC(C)(C)OC(=O)C1CCN(c2ccc([C@H]3CCC(=O)NC3=O)cc2)CC1]

A mixture of tert-butyl 1-(4-(2,6-bis(benzyloxy)pyridin-3-yl)phenyl)piperidine-4-carboxylate (75.0 g, 136.1 mmol) and Pd/C (15.0 g, 14.1 mmol, 10% purity), and Pd(OH)₂ (15.0 g, 21.3 mmol, 20% purity) in THF (750 mL) was purged of air and back filled with N₂ (3×). The vessel was then purged of N₂ and backfilled with H₂ (3×), then stirred at 50° C. until complete as judged by LCMS. Once complete, the mixture was filtered through celite, and the filtrate was concentrated under vacuum afford the desired compound, which was taken forward without any further purification (96 g). LCMS C₂₁H₂₈N₂O₄ requires 372.2, found m/z=373.3 [M+H]⁺.

### Step 3: Synthesis of rac-(R)-1-(4-(2,6-dioxopiperidin-3-yl)phenyl)piperidine-4-carboxylic acid

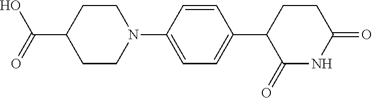 [SMILES: O=C1CCC(c2ccc(N3CCC(C(=O)O)CC3)cc2)C(=O)N1]

To a solution of rac-tert-butyl (R)-1-(4-(2,6-dioxopiperidin-3-yl)phenyl)piperidine-4-carboxylate (45.0 g, 120 mmol) in DCM (500 mL) was slowly added TFA (179 mL, 2.42 mol) at 0° C., and the mixture was stirred at 30° C. until complete as judged by LCMS. Once complete, the mixture was concentrated to give a crude residue. The residue was purified by RP-FC (5-95%) to afford the desired compound as an off-white solid (40.3 g, 75% yield over two steps). LCMS C₁₇H₂₀N₂O₄ requires 316.2, found m/z=317.1 [M+H]⁺.

### Step 4: Synthesis of rel-tert-butyl (R)-1-(4-(2,6-dioxopiperidin-3-yl)phenyl)piperidine-4-carboxylate

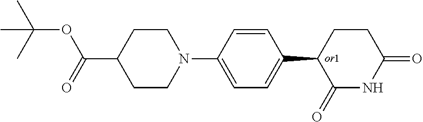 [SMILES: CC(C)(C)OC(=O)C1CCN(c2ccc([C@H]3CCC(=O)NC3=O)cc2)CC1]

rac-tert-butyl (R)-1-(4-(2,6-dioxopiperidin-3-yl)phenyl)piperidine-4-carboxylate was purified by SFC (DAICEL CHIRALPAK AS (250 mm*30 mm, 10 um); mobile phase: [CO2-i-PrOH/ACN]; B %: 35%, isocratic elution mode). The desired compound was obtained as a white solid (first-eluting isomer, 20.0 g, 40% yield). LCMS C₂₁H₂₈N₂O₄ requires 372.2, found 373.4 [M+H]⁺.

### Step 5: Synthesis of rel-tert-butyl (R)-1-(4-(2,6-dioxopiperidin-3-yl)phenyl)piperidine-4-carboxylate

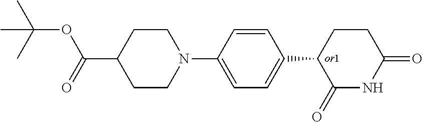 [SMILES: CC(C)(C)OC(=O)C1CCN(c2ccc([C@@H]3CCC(=O)NC3=O)cc2)CC1]

rac-tert-butyl (R)-1-(4-(2,6-dioxopiperidin-3-yl)phenyl)piperidine-4-carboxylate was purified by SFC (DAICEL CHIRALPAK AS (250 mm*30 mm, 10 um); mobile phase: [CO2-i-PrOH/ACN]; B %: 35%, isocratic elution mode). The desired compound was obtained as a white solid (second-eluting isomer, 19.0 g, 37% yield). LCMS C₂₁H₂₈N₂O₄ requires 372.2, found 373.4 [M+H]⁺.

### Step 6: Synthesis of rel-(R)-1-(4-(2,6-dioxopiperidin-3-yl)phenyl)piperidine-4-carboxylic acid

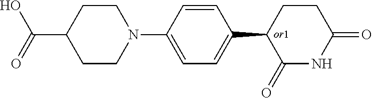 [SMILES: O=C1CC[C@H](c2ccc(N3CCC(C(=O)O)CC3)cc2)C(=O)N1]

This compound was prepared as described in Step 3 for Intermediate 41, using rel-tert-butyl (R)-1-(4-(2,6-dioxopiperidin-3-yl)phenyl)piperidine-4-carboxylate in place of rac-tert-butyl (R)-1-(4-(2,6-dioxopiperidin-3-yl)phenyl)piperidine-4-carboxylate (22 g, 93% yield). LCMS C₁₇H₂₀N₂O₄ requires 316.1, found m/z=317.2 [M+H]⁺.

### Synthesis of Title Compound

This compound was prepared as described in Step 3 for Intermediate 41, using rec-tert-butyl (R)-1-(4-(2,6-dioxopiperidin-3-yl)phenyl)piperidine-4-carboxylate in place of rac-tert-butyl (R)-1-(4-(2,6-dioxopiperidin-3-yl)phenyl)piperidine-4-carboxylate (14.2 g, 94% yield). LCMS C₁₇H₂₀N₂O₄ requires 316.1, found m/z=317.2 [M+H]⁺.

### EXAMPLES

### Purification Procedures

Preparative-scale HPLC was performed using columns such as SunFire Prep C18 OBD, XBridge Prep OBD C18 and Xbridge Shield RP18 OBD, using solvent systems such as (water-0.1% formic acid)/acetonitrile, (water-10 mmol/L NH₄HCO₃)/acetonitrile, or (water-10 mmol/L NH₄HCO₃)/acetonitrile. Chromatography A refers to purification over silica gel, typically in pre-packed cartridges, eluting with mixtures of EtOAc in hexanes or petroleum ether; Chromatography B refers to elution with mixtures of MeOH in DCM; Chromatography C refers to use of C18 reverse-phase silica gel, eluting with mixtures of acetonitrile in water. Compounds drawn without stereochemistry were tested as racemic or diasteromeric mixtures in the Biological Examples.

Abbreviations Abbreviations used in the Examples include the following: BOP (Benzotriazol-1-yloxy)tris(dimethylamino)phosphonium hexafluorophosphate; BINAP (2,2-bis(diphenylphosphino)-1,1-binaphthyl); Bn (benzyl), Boc (tert-butoxycarbonyl); CBz: (benzyloxycarbonyl), HATU (N-[(Dimethylamino)-1H-1,2,3-triazolo-[4,5-b]pyridin-1-ylmethylene]-N-methylmethanaminium hexafluorophosphate N-oxide); DMSO (dimethyl sulfoxide); THF (tetrahydrofuran); EtOAc (ethyl acetate); ACN (acetonitrile); Et₂O (diethyl ether); DCM (dichloromethane); MeOH (methanol); EtOH (ethanol); DCE (1,2-dichloroethane); TEA (trimethylamine); TFA (trifluoroacetic acid); DIEA (N,N-diisopropylethlamine); DIPEA (N,N-Diisopropylethylamine); DMF (N,N-dimethylformamide); NMP (N-methyl-2-pyrrolidone); N,N-dimethylacetamide (DMA); EDCI (1-ethyl-3-(3-dimethylaminopropyl)carbodiimide); HFIP (hexafluoroisopropanol); HOBT (hydroxybenzotriazole); STAB (sodium triacetoxyborohydride); Pd₂(dba)₃ (tris(dibenzylideneacetone)dipalladium); Pd(dppf)Cl₂ ([1,1′-bis(diphenylphosphino)ferrocene]dichloropalladium(II)); PE (petroleum ether); RuPhos (palladacycle Gen.3: Methanesulfonato(2-dicyclohexylphosphino-2′,6′-bis(dimethylamino)-1,1′-biphenyl)(2′-amino-1,1′-biphenyl-2-yl)palladium(II)); SFC (supercritical fluid chromatography); T₃P (propanephosphonic acid cyclic anhydride); XantPhos (9,9-dimethyl-4,5-bis(diphenylphosphino)xanthene); TFA (trifluoroacetic acid); rt or RT (room temperature); anh (anhydrous); eq. or equiv. (equivalent), FC (flash chromatography).

### Example 1

### rac-N-[(3r)-2,6-dioxopiperidin-3-yl]-4-(4-{1-[6-(2-hydroxyphenyl)pyridazin-4-yl]-4-phenylpiperidine-4-carbonyl}piperazin-1-yl)pyridine-2-carboxamide

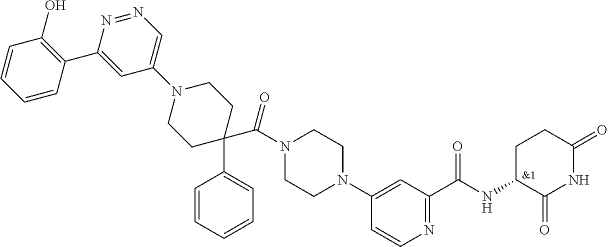 [SMILES: O=C1CC[C@@H](NC(=O)c2cc(N3CCN(C(=O)C4(c5ccccc5)CCN(c5cnnc(-c6ccccc6O)c5)CC4)CC3)ccn2)C(=O)N1]

### Step 1: Synthesis of methyl 1-(6-chloropyridazin-4-yl)-4-phenylpiperidine-4-carboxylate

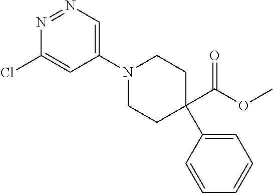 [SMILES: COC(=O)C1(c2ccccc2)CCN(c2cnnc(Cl)c2)CC1]

A vial was charged with 5-bromo-3-chloropyridazine (0.50 g, 2.5849 mmol), methyl 4-phenylpiperidine-4-carboxylate (0.57 g, 2.5849 mmol), and N,N-diisopropylethylamine (1.81 mL, 1.34 g, 10.3397 mmol) in DMSO. This solution was heated to 120 C overnight and then purified directly via RP-FC to yield the title compound (750 mg, 86%). LCMS: C₁₇H₁₈ClN₃O₂ requires: 331.2. found: m/z=332.4 [M+H]⁺.

### Step 2: Synthesis of methyl 1-(6-(2-hydroxyphenyl)pyridazin-4-yl)-4-phenylpiperidine-4-carboxylate

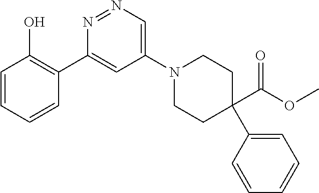 [SMILES: COC(=O)C1(c2ccccc2)CCN(c2cnnc(-c3ccccc3O)c2)CC1]

To a flask equipped with a stir bar was added methyl 1-(6-chloropyridazin-4-yl)-4-phenylpiperidine-4-carboxylate (500.00 mg, 1.5069 mmol), tetrakis(triphenylphosphine)palladium(0) (0.17 g, 0.1507 mmol), potassium carbonate (0.83 g, 6.0277 mmol), and 2-hydroxyphenylboronic acid (207.85 mg, 1.5069 mmol). To these dry powders was added 12 mL of dry and pre-sparged 1,4-dioxane. The vial was sealed and allowed to heat to 95 C overnight. Upon completion, the crude mixture was filtered through celite and purified by RP-FC to furnish the title compound (70 mg, 12%). LCMS: C₂₃H₂₃N₃O₃ requires: 389.5. found: m/z=390.5 [M+H]⁺.

### Step 3: Synthesis of 1-(6-(2-hydroxyphenyl)pyridazin-4-yl)-4-phenylpiperidine-4-carboxylic acid

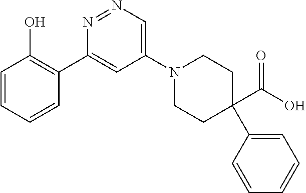 [SMILES: O=C(O)C1(c2ccccc2)CCN(c2cnnc(-c3ccccc3O)c2)CC1]

Dissolve methyl 1-(6-(2-hydroxyphenyl)pyridazin-4-yl)-4-phenylpiperidine-4-carboxylate (200 mg, 0.5135 mmol) in 2 mL of 1:1 Dioxane: 1M NaOH. Stir until complete, about 3 hours, then filter and purify directly via RP-FC to furnish the title compound in quantitative yield. LCMS: C₂₂H₂₁N₃O₃ requires: 375.4. found: m/z=376.4 [M+H]⁺.

### Step 4: Synthesis of 4-fluoropicolinic acid

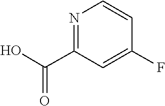

To a solution of methyl 4-fluoropicolinate (10.0 g, 64.4 mmol, 1.00 eq) in THF (100 mL) and H₂O (50.0 mL) was added NaOH (3.87 g, 96.7 mmol, 1.50 eq) at 25° C. Then the mixture was stirred at 25° C. for 2 hrs. The mixture was adjusted to pH=2-3 with 1N HCl and lyophilized without purification. 4-fluoropicolinic acid (18.0 g, 122 mmol, 94.8% yield, 95.8% purity) was obtained as a white solid. LCMS C₆H₄FNO₂ requires: 141.0. found: m/z=141.1 [M+H]⁺.

¹H NMR: (400 MHz, DMSO) δ 8.70 (dd, J=8.4, 5.6 Hz, 1H), 7.83 (dd, J=9.6, 2.4 Hz, 1H), 7.54 (ddd, J=8.8, 5.6, 2.4 Hz, 1H).

### Step 5: Synthesis of rac-(R)-N-(2,6-dioxopiperidin-3-yl)-4-fluoropicolinamide

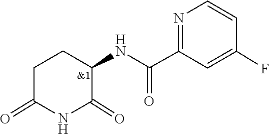 [SMILES: O=C1CC[C@@H](NC(=O)c2cc(F)ccn2)C(=O)N1]

A solution of 4-fluoropicolinic acid (9.00 g, 61.1 mmol, 95.8% purity, 1.00 eq) in SOCl₂ (147 g, 1.24 mol, 90.0 mL, 20.3 eq) was stirred at 90° C. for 1 hr. The mixture was concentrated to remove SOCl₂. The residue was dissolved in DCM (200 mL). The mixture was added dropwise to a solution of 3-aminopiperidine-2,6-dione (10.1 g, 61.1 mmol, 1.00 eq, HCl) and NEt₃ (30.9 g, 305 mmol, 42.5 mL, 5.00 eq) in DCM (200 mL) at 0° C. Then the mixture was stirred at 25° C. for 12 hrs. The mixture was concentrated under vacuum to obtain the crude product without purification. rac-(R)-N-(2,6-dioxopiperidin-3-yl)-4-fluoropicolinamide (19.0 g, 58.1 mmol, 95.1% yield, 76.8% purity) was obtained as a blue solid. LCMS C₁₁H₁₀FN₃O₃ requires: 251.1. found: m/z=252.0 [M+H]⁺. ¹H NMR (400 MHz, DMSO) δ 10.89 (br s, 1H), 9.16 (d, J=8.5 Hz, 1H), 8.67-8.82 (m, 1H), 7.81-7.91 (m, 1H) 7.60 (ddd, J=8.8, 5.6, 2.8 Hz, 1H), 4.69-4.89 (m, 1H), 3.38 (br d, J=7.2 Hz, 1H), 2.74-2.87 (m, 1H), 2.53-2.59 (m, 1H), 2.23 (qd, J=13.2, 4.4 Hz, 1H), 2.00 (dtd, J=12.8, 5.2, 5.2, 2.4 Hz, 1H).

### Step 6: Synthesis of rac-tert-butyl (R)-4-(2-((2,6-dioxopiperidin-3-yl)carbamoyl)pyridin-4-yl)piperazine-1-carboxylate

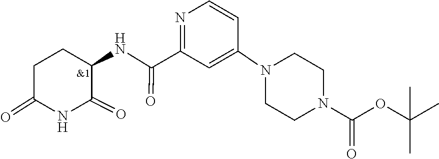 [SMILES: CC(C)(C)OC(=O)N1CCN(c2ccnc(C(=O)N[C@@H]3CCC(=O)NC3=O)c2)CC1]

To a solution of rac-(R)-N-(2,6-dioxopiperidin-3-yl)-4-fluoropicolinamide (17.0 g, 51.9 mmol, 76.8% purity, 1.00 eq) and tert-butyl piperazine-1-carboxylate (9.68 g, 51.9 mmol, 1.00 eq) in DMF (170 mL) was added DIEA (26.8 g, 207 mmol, 36.2 mL, 4.00 eq). Then the mixture was stirred at 100° C. for 12 hrs. The mixture was diluted with water (500 mL) and extracted with ethyl acetate (3×250 mL). The combined organic layer was washed with brine (100 mL), dried over Na₂SO₄, filtered, and concentrated under vacuum to get the crude product. The crude product was purified by column chromatography (SiO₂, petroleum ether/ethyl acetate=100/1 to 1/1). rac-tert-butyl (R)-4-(2-((2,6-dioxopiperidin-3-yl)carbamoyl)pyridin-4-yl)piperazine-1-carboxylate (1.86 g, 3.98 mmol, 7.66% yield, 89.3% purity) was obtained as a yellow solid. LCMS C₂₀H₂₇N₅O₅ requires: 417.2. found: m/z=418.3 [M+H]⁺.

¹H NMR (400 MHz, DMSO) δ 10.85 (s, 1H), 8.96 (d, J=8.4 Hz, 1H), 8.24 (d, J=6.0 Hz, 1H), 7.46 (d, J=2.8 Hz, 1H), 7.00 (dd, J=6.0, 2.8 Hz, 1H), 4.69-4.80 (m, 1H), 3.44 (br dd, J=14.8, 5.6 Hz, 8H), 2.74-2.85 (m, 1H), 2.13-2.24 (m, 1H), 1.96-2.04 (m, 1H), 1.42 (s, 9H)

### Step 7: Synthesis of rac-(R)-N-(2,6-dioxopiperidin-3-yl)-4-(piperazin-1-yl)picolinamide

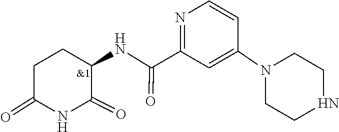 [SMILES: O=C1CC[C@@H](NC(=O)c2cc(N3CCNCC3)ccn2)C(=O)N1]

To a solution of rac-tert-butyl (R)-4-(2-((2,6-dioxopiperidin-3-yl)carbamoyl)pyridin-4-yl)piperazine-1-carboxylate (1.86 g, 3.98 mmol, 89.3% purity, 1.00 eq) in DCM (18.0 mL) was added HCl/dioxane (4.00 M, 19.6 mL, 19.7 eq) at 25° C. Then the mixture was stirred at 25° C. for 12 hrs. The mixture was filtered and the cake was concentrated to get the crude product without purification. rac-(R)-N-(2,6-dioxopiperidin-3-yl)-4-(piperazin-1-yl)picolinamide (1.50 g, 3.83 mmol, 96.3% yield, 90.4% purity, HCl salt) was obtained as an off-white solid. LCMS C₁₅H₁₉N₅O₃ requires: 317.1. found: m/z=318.1 [M+H]⁺.

¹H NMR (400 MHz, D₂O) δ 8.25 (d, J=7.2 Hz, 1H), 7.73 (d, J=2.8 Hz, 1H), 7.25 (dd, J=7.2, 2.8 Hz, 1H), 4.89-5.00 (m, 1H), 3.93-4.13 (m, 4H), 3.72 (s, 1H), 3.40-3.51 (m, 4H), 2.72-2.93 (m, 2H), 2.17-2.37 (m, 2H).

### Step 8: Synthesis of the Title Compound

Stirred 1-[6-(2-hydroxyphenyl)pyridazin-4-yl]-4-phenylpiperidine-4-carboxylic acid (10.00 mg, 0.0266 mmol), [(dimethylamino)({[1,2,3]triazolo[4,5-b]pyridin-3-yloxy})methylidene]dimethylazanium; hexafluoro-lambda5-phosphanuide (20.26 mg, 0.0533 mmol), and N,N-diisopropylethylamine (13.77 mg, 0.1065 mmol) in DMF. Added rac-(R)-N-(2,6-dioxopiperidin-3-yl)-4-(piperazin-1-yl)picolinamide (8.45 mg, 0.0266 mmol) and let stir at r.t. until complete by LCMS. The solution was injected onto RP-FC and purified with a gradient of 0-70% MeCN in H₂O to furnish the title compound (2.7 mg, 15%). LCMS: C₃₇H₃₈N₅O₅ requires: 674.3, found: m/z=675.3 [M+H]⁺.

### Example 2

### (3RS)-3-{4-[(1S)-1-(1-{1-[6-(2-hydroxyphenyl)pyridazin-4-yl]-4-phenylpiperidine-4-carbonyl}piperidin-4-yl)ethoxy]phenyl}piperidine-2,6-dione

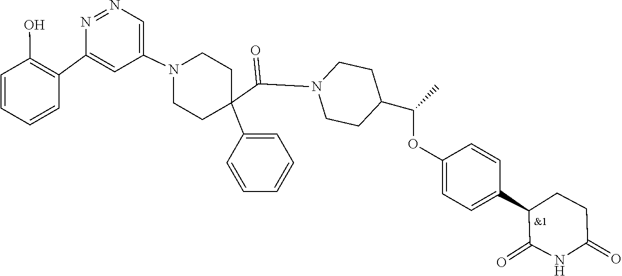 [SMILES: C[C@H](Oc1ccc([C@H]2CCC(=O)NC2=O)cc1)C1CCN(C(=O)C2(c3ccccc3)CCN(c3cnnc(-c4ccccc4O)c3)CC2)CC1]

### Step 1: Synthesis of tert-butyl (S)-4-(1-(4-(2,6-bis(benzyloxy)pyridin-3-yl)phenoxy)ethyl)piperidine-1-carboxylate

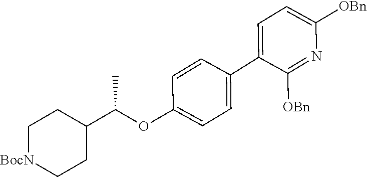 [SMILES: C[C@H](Oc1ccc(-c2ccc(OCc3ccccc3)nc2OCc2ccccc2)cc1)C1CCN(C(=O)OC(C)(C)C)CC1]

To a 20 mL vial was added 4-[2,6-bis(benzyloxy)pyridin-3-yl]phenol (500.00 mg, 1.3040 mmol) synthesized as described for compound 3 in PCT publication WO2023/018238, tert-butyl 4-[(1R)-1-hydroxyethyl]piperidine-1-carboxylate (897.07 mg, 3.9119 mmol), triphenylphosphine (1.03 g, 3.9119 mmol), and THF (10.00 mL). The reaction mixture was cooled to 0 C, then diisopropyl azodicarboxylate (0.77 mL, 0.79 g, 3.9119 mmol) was added in a dropwise fashion. The reaction mixture was stirred warming to RT for 16 h, then concentrated. The resulting residue was purified by FC (80 g silica, 0-25% EtOAc/hex) to yield the title compound as a colorless oil (415 mg, 54%). LCMS: C₃₇H₄₂N₂O₅ requires: 594.7. found: m/z=595.7 [M+H]⁺.

### Step 2: Synthesis of (3RS)-3-{4-[(1S)-1-(piperidin-4-yl)ethoxy]phenyl}piperidine-2,6-dione

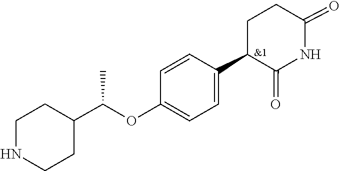 [SMILES: C[C@H](Oc1ccc([C@H]2CCC(=O)NC2=O)cc1)C1CCNCC1]

To a 20 mL vial was added tert-butyl 4-[(1S)-1-{4-[2,6-bis(benzyloxy)pyridin-3-yl]phenoxy}ethyl]piperidine-1-carboxylate (415.00 mg, 0.6978 mmol), Pd/C (400.00 mg, mmol), EtOH (5.00 mL), and THF (5.00 mL). The reaction mixture was sparged with H₂ for 10 min, then stirred under H₂ atmosphere (balloon) for 16 h. The reaction mixture was then filtered through a pad of celite, then concentrated. The crude material was dissolved in TFA/DCM 1:1 and stirred for 30 minutes, then concentrated and lyophilized to yield the title compound as a white solid (247 mg, 85%). LCMS: C₁₈H₂₄N₂O₃ requires: 316.2. found: m/z=317.5 [M+H]⁺.

### Step 3: Synthesis of the Title Compound

Stirred 1-[6-(2-hydroxyphenyl)pyridazin-4-yl]-4-phenylpiperidine-4-carboxylic acid (10.00 mg, 0.0266 mmol), [(dimethylamino)({[1,2,3]triazolo[4,5-b]pyridin-3-yloxy})methylidene]dimethylazanium; hexafluoro-lambda5-phosphanuide (20.26 mg, 0.0533 mmol), and N,N-diisopropylethylamine (13.77 mg, 0.1065 mmol) in DMF. Add rac-N-[(3R)-2,6-dioxopiperidin-3-yl]-4-(piperazin-1-yl)pyridine-2-carboxamide (8.45 mg, 0.0266 mmol) and let stir at r.t. until complete by LCMS. The solution was injected onto RP-FC and purified with a gradient of 0-70% MeCN in H₂O to furnish the title compound (6.5 mg, 33%). LCMS: C₄₀H₄₃N₅O₅ requires: 673.3. found: m/z=674.3 [M+H]⁺.

### Example 3

### (3RS)-3-{4-[(1R)-1-(1-{1-[6-(2-hydroxyphenyl)pyridazin-4-yl]-4-phenylpiperidine-4-carbonyl}piperidin-4-yl)ethoxy]phenyl}piperidine-2,6-dione

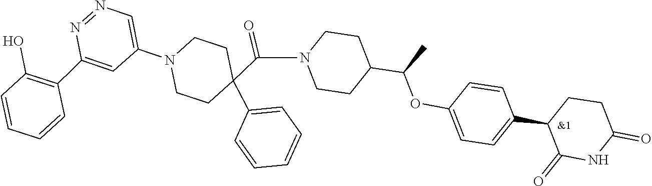 [SMILES: C[C@@H](Oc1ccc([C@H]2CCC(=O)NC2=O)cc1)C1CCN(C(=O)C2(c3ccccc3)CCN(c3cnnc(-c4ccccc4O)c3)CC2)CC1]

Stirred 1-[6-(2-hydroxyphenyl)pyridazin-4-yl]-4-phenylpiperidine-4-carboxylic acid (11.00 mg, 0.0293 mmol), [(dimethylamino)({[1,2,3]triazolo[4,5-b]pyridin-3-yloxy})methylidene]dimethylazanium; hexafluoro-lambda5-phosphanuide (22.28 mg, 0.0586 mmol), and N,N-diisopropylethylamine (20.47 μL, 15.15 mg, 0.1172 mmol) in DMF. Add (3RS)-3-{4-[(1R)-1-(piperidin-4-yl)ethoxy]phenyl}piperidine-2,6-dione (9.27 mg, 0.0293 mmol), prepared according to the inversed methyl diastereomer of Step 2, Example 2, and let stir at r.t. until complete by LCMS. The solution was injected onto RP-FC and purified with a gradient of 0-70% MeCN in H₂O to furnish the title compound (4.6 mg, 23%). LCMS: C₄₀H₄₃N₅O₅ requires: 673.3. found: m/z=674.3 [M+H]⁺.

### Example 4

### rac-(3R)-3-{4-[(1-{1-[6-(2-hydroxyphenyl)pyridazin-4-yl]-4-phenylpiperidine-4-carbonyl}piperidin-4-yl)oxy]phenyl}piperidine-2,6-dione

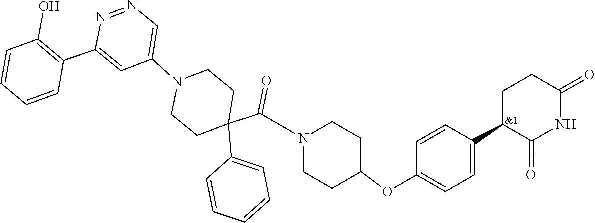 [SMILES: O=C1CC[C@H](c2ccc(OC3CCN(C(=O)C4(c5ccccc5)CCN(c5cnnc(-c6ccccc6O)c5)CC4)CC3)cc2)C(=O)N1]

### Step 1: Synthesis of 4-(2,6-bis(benzyloxy)pyridin-3-yl)phenol

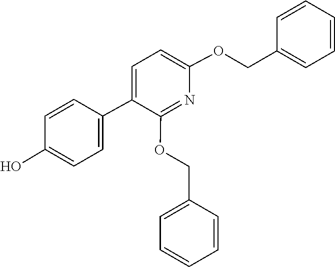 [SMILES: Oc1ccc(-c2ccc(OCc3ccccc3)nc2OCc2ccccc2)cc1]

Dissolved 2,6-bis(benzyloxy)-3-bromopyridine (290 g, 783 mmol, 1.00 eq) and (4-hydroxyphenyl)boronic acid (118 g, 861 mmol, 1.10 eq) in dioxane (2.90 L). Charged K₂CO₃ (216 g, 1.57 mol, 2.00 eq) and H₂O (580 mL) into the reactor under N₂. The suspension was degassed under vacuum and purged with N₂ several times. Charged Pd(dppf)Cl₂ (28.6 g, 39.1 mmol, 0.05 eq) into the reactor under N₂. The suspension was degassed under vacuum and purged with N₂ several times. Stirred for 12 hrs at 110° C. The reaction solution was concentrated under reduced pressure, then diluted with water (500 mL) and extracted with ethyl acetate (3×500 mL). The organic layer was washed twice with brine (500 mL), dried over Na₂SO₄, filtered, and concentrated under reduced pressure. The crude product was purified by column chromatography (SiO₂, N-heptane/ethyl acetate=100/1 to 10/1). 4-(2,6-bis(benzyloxy)pyridin-3-yl)phenol (240 g, 597 mmol, 76.3% yield, 95.5% purity) was obtained as an off-white solid.

¹H NMR (400 MHz, DMSO) δ ppm 9.45 (s, 1H) 7.65 (d, J=8.07 Hz, 1H) 7.20-7.46 (m, 12H) 6.77 (d, J=8.56 Hz, 2H) 6.51 (d, J=8.07 Hz, 1H) 5.37 (d, J=13.0 Hz, 4H).

### Step 2: Synthesis of benzyl 4-(4-(2,6-bis(benzyloxy)pyridin-3-yl)phenoxy)piperidine-1-carboxylate

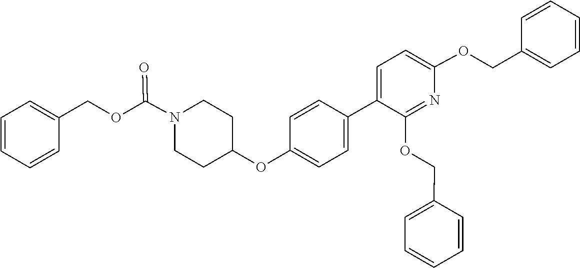 [SMILES: O=C(OCc1ccccc1)N1CCC(Oc2ccc(-c3ccc(OCc4ccccc4)nc3OCc3ccccc3)cc2)CC1]

Charged 4-(2,6-bis(benzyloxy)pyridin-3-yl)phenol (120 g, 313 mmol, 1.00 eq) into THF (840 mL) in a reactor R-1 (2.00 L bottle, in parallel) set up with an agitator. Charged benzyl 4-hydroxypiperidine-1-carboxylate (77.3 g, 328 mmol, 1.05 eq) into R-1. Charged PPh₃ (86.1 g, 328 mmol, 1.05 eq) into R-1. Added DEAD (57.2 g, 328 mmol, 59.7 mL, 1.05 eq) at 0° C. under nitrogen atmosphere. Stirred the mixture at 25° C. for 12 hrs under N₂. The reaction solution was concentrated under reduced pressure. The crude product was triturated with MTBE (500 mL) at 20° C. for 1 hr. The filtrate was concentrated under vacuum at 40-45° C. Then the crude product was purified by prep-HPLC (column: Phenomenex luna C18 (250×70 mm, 10 um); mobile phase: [water (HCl)-ACN]; B %: 100-100% 40 min). benzyl 4-(4-(2,6-bis(benzyloxy)pyridin-3-yl)phenoxy)piperidine-1-carboxylate (260 g, 427 mmol, crude) was obtained as a yellow oil. LCMS: C₃₈H₃₆N₂O₅ requires: 600.3. found: m/z=601.4 [M+H]⁺.

¹H NMR: (400 MHz, DMSO) δ ppm 7.69 (d, J=8.07 Hz, 1H) 7.24-7.50 (m, 17H) 6.99 (d, J=8.68 Hz, 2H) 6.52 (d, J=8.07 Hz, 1H) 5.38 (d, J=15.1 Hz, 4H) 5.09 (s, 2H) 4.60 (dt, J=7.55, 3.99 Hz, 1H) 3.69-3.79 (m, 2H) 3.30 (br d, J=5.99 Hz, 2H) 1.88-1.97 (m, 2H) 1.56 (ddt, J=12.6, 8.44, 4.29, 4.29 Hz, 2H).

### Step 3: Synthesis of 5-(4-(piperidin-4-yloxy)phenyl)pyridine-2,6(1H,3H)-dione

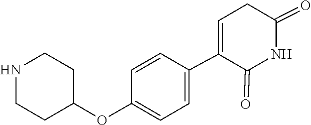 [SMILES: O=C1CC=C(c2ccc(OC3CCNCC3)cc2)C(=O)N1]

Charged benzyl 4-(4-(2,6-bis(benzyloxy)pyridin-3-yl)phenoxy)piperidine-1-carboxylate (100 g, 166 mmol, 1.00 eq) into DCM (500 mL) in a reactor R-1 (2.00 L bottle, in parallel) set up with an agitator. Charged HBr (521 g, 2.13 mol, 350 mL, 33% purity, 12.7 eq) at 20° C. into R-1. Stirred the mixture at 50° C. for 12 hrs under N₂. The reaction solution was concentrated under reduced pressure. 5-(4-(piperidin-4-yloxy)phenyl)pyridine-2,6(1H,3H)-dione (40.0 g, 139 mmol, 83.9% yield, 95.2% purity) was obtained as a yellow oil. LCMS: C₁₆H₁₈N₂O₃ requires: 286.1. found: m/z=286.9 [M+H]⁺.

### Step 4: Synthesis of rac-(R)-3-(4-(piperidin-4-yloxy)phenyl)piperidine-2,6-dione

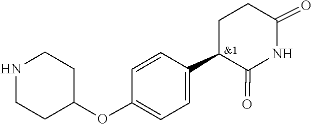 [SMILES: O=C1CC[C@H](c2ccc(OC3CCNCC3)cc2)C(=O)N1]

Set up a reactor R-1 (2.00 L bottle) with an agitator. Blew argon into the bottle for 2 min. Added Pd(OH)₂ (20.0 g, 14.2 mmol, 10% purity, 0.12 eq) and Pd/C (20.0 g, 18.7 mmol, 10% purity, 0.13 eq) soaked with MeOH (100 mL) into R-1. Charged 5-(4-(piperidin-4-yloxy)phenyl)pyridine-2,6(1H,3H)-dione (40.0 g, 139 mmol, 1.00 eq) with MeOH (700 mL) into R-1. Hydrogen was replaced three times and stirred at 50° C. for 12 hrs. The reaction was filtered and washed with MeOH (2.00 L). HCl/EA (120 mL) was added dropwise. rac-(R)-3-(4-(piperidin-4-yloxy)phenyl)piperidine-2,6-dione (30.0 g, 90.3 mmol, 64.6% yield, 97.8% purity, HCl salt) was obtained as a white solid. LCMS: C₁₆H₂₀N₂O₃ requires: 288.1. found: m/z=288.9 [M+H]⁺. ¹H NMR: (400 MHz, DMSO) δ ppm 10.7 (s, 1H) 9.18-9.39 (m, 2H) 7.14 (d, J=8.58 Hz, 2H) 6.95 (d, J=8.70 Hz, 2H) 4.63 (dt, J=7.12, 3.77 Hz, 1H) 3.79 (dd, J=11.5, 4.83 Hz, 1H) 3.12-3.28 (m, 2H) 2.98-3.11 (m, 2H) 2.60-2.72 (m, 1H) 2.45 (br t, J=3.99 Hz, 1H) 2.06-2.22 (m, 3H) 1.95-2.04 (m, 1H) 1.80-1.90 (m, 2H)

### Step 5: Synthesis of the Title Compound

Stirred 1-[6-(2-hydroxyphenyl)pyridazin-4-yl]-4-phenylpiperidine-4-carboxylic acid (5.00 mg, 0.0133 mmol), [(dimethylamino)({[1,2,3]triazolo[4,5-b]pyridin-3-yloxy})methylidene]dimethylazanium; hexafluoro-lambda5-phosphanuide (10.13 mg, 0.0266 mmol), and N,N-diisopropylethylamine (9.30 μL, 6.89 mg, 0.0533 mmol) in DMF. Added rac-(3R)-3-[4-(piperidin-4-yloxy)phenyl]piperidine-2,6-dione (3.84 mg, 0.0133 mmol) and let stir at r.t. until complete by LCMS. The solution was injected onto RP-FC and purified with a gradient of 0-70% MeCN in H₂O to furnish the title compound (2.0 mg, 14%). LCMS: C₃₈H₃₉N₅O₅ requires: 645.3, found: m/z=646.3 [M+H]⁺.

### Example 5

### rac-(3R)-3-{4-[(1-{1-[6-(2-hydroxyphenyl)pyridazin-4-yl]-4-phenylpiperidine-4-carbonyl}piperidin-4-yl)methoxy]phenyl}piperidine-2,6-dione

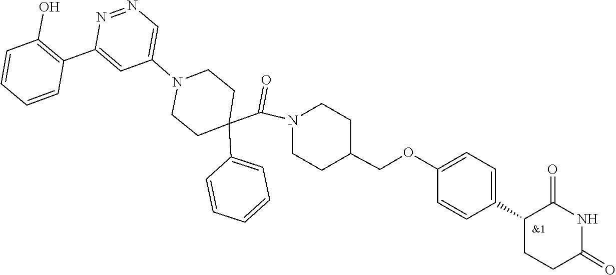 [SMILES: O=C1CC[C@H](c2ccc(OCC3CCN(C(=O)C4(c5ccccc5)CCN(c5cnnc(-c6ccccc6O)c5)CC4)CC3)cc2)C(=O)N1]

### Step 1: Synthesis of tert-butyl 4-((4-(2,6-bis(benzyloxy)pyridin-3-yl)phenoxy)methyl)piperidine-1-carboxylate

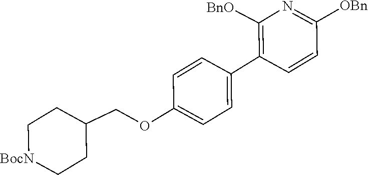 [SMILES: CC(C)(C)OC(=O)N1CCC(COc2ccc(-c3ccc(OCc4ccccc4)nc3OCc3ccccc3)cc2)CC1]

To a 20 mL vial was added 4-[2,6-bis(benzyloxy)pyridin-3-yl]phenol (200.00 mg, 0.5216 mmol), tert-butyl 4-(hydroxymethyl)piperidine-1-carboxylate (336.88 mg, 1.5648 mmol), triphenylphosphine (0.41 g, 1.5648 mmol), and THF (4.00 mL). The reaction mixture was cooled to 0 C, then diisopropyl azodicarboxylate (0.31 mL, 0.32 g, 1.5648 mmol) was added in a dropwise fashion. The reaction mixture was stirred warming to RT for 16 h, then concentrated. The resulting residue was purified by FC (40 g silica, 0-25% EtOAc/hex) to furnish the title compound as a colorless oil (277 mg, 92%): C₃₆H₄₀N₂O₅ requires: 580.6. found: m/z=581.5 [M+H]⁺.

### Step 2: Synthesis of rac-(R)-3-(4-(piperidin-4-ylmethoxy)phenyl)piperidine-2,6-dione

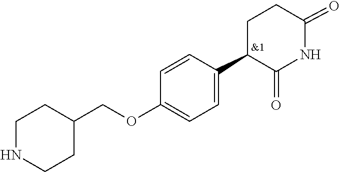 [SMILES: O=C1CC[C@H](c2ccc(OCC3CCNCC3)cc2)C(=O)N1]

To a 20 mL vial was added tert-butyl 4-{4-[2,6-bis(benzyloxy)pyridin-3-yl]phenoxymethyl}piperidine-1-carboxylate (277.00 mg, 0.4770 mmol), Pd/C (100.00 mg, mmol), EtOH (2.00 mL), and THF (2.00 mL). The reaction mixture was sparged with H₂ for 10 min, then stirred under H₂ atmosphere (balloon) for 16 h. The reaction mixture was then filtered through a pad of celite, then concentrated. To the crude product was added 4N hydrogen chloride in dioxane (1.00 mL, 0.15 g, 4.0000 mmol). The reaction mixture was stirred for 1 h, then concentrated. LCMS: C₁₇H₂₂N₂O₃ requires: 302.2. found: m/z=303.3 [M+H]⁺.

### Step 3: Synthesis of the Title Compound

Stirred 1-[6-(2-hydroxyphenyl)pyridazin-4-yl]-4-phenylpiperidine-4-carboxylic acid (5.00 mg, 0.0133 mmol), [(dimethylamino)({[1,2,3]triazolo[4,5-b]pyridin-3-yloxy})methylidene]dimethylazanium; hexafluoro-lambda5-phosphanuide (10.13 mg, 0.0266 mmol), and N,N-diisopropylethylamine (9.30 μL, 6.89 mg, 0.0533 mmol) in DMF. Add rac-(3R)-3-[4-(piperidin-4-ylmethoxy)phenyl]piperidine-2,6-dione (4.03 mg, 0.0133 mmol) and let stir at r.t. until complete by LCMS. The solution was injected onto RP-FC and purified with a gradient of 0-70% MeCN in H₂O to furnish the title compound (1.2 mg, 14%). LCMS: C₃₉H₄₁N₅O₅ requires: 659.3. found: m/z=660.3 [M+H]⁺.

### Example 6

### rac-(3R)-3-(4-{4-[(4-{1-[6-(2-hydroxyphenyl)pyridazin-4-yl]-4-phenylpiperidine-4-carbonyl}piperazin-1-yl)methyl]piperidin-1-yl}phenyl)piperidine-2,6-dione

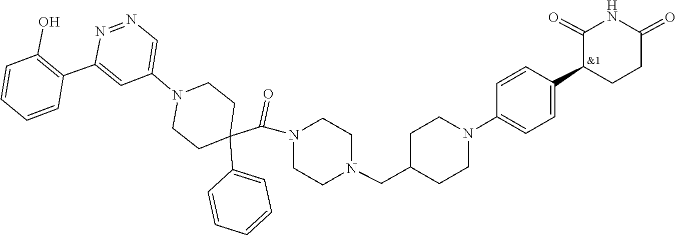 [SMILES: O=C1CC[C@@H](c2ccc(N3CCC(CN4CCN(C(=O)C5(c6ccccc6)CCN(c6cnnc(-c7ccccc7O)c6)CC5)CC4)CC3)cc2)C(=O)N1]

Stirred 1-[6-(2-hydroxyphenyl)pyridazin-4-yl]-4-phenylpiperidine-4-carboxylic acid (5.00 mg, 0.0133 mmol), [(dimethylamino)({[1,2,3]triazolo[4,5-b]pyridin-3-yloxy})methylidene]dimethylazanium; hexafluoro-lambda5-phosphanuide (10.13 mg, 0.0266 mmol), and N,N-diisopropylethylamine (9.30 μL, 6.89 mg, 0.0533 mmol) in DMF. Added rac-(3R)-3-{4-[4-(piperazin-1-ylmethyl)piperidin-1-yl]phenyl}piperidine-2,6-dione (5.43 mg, 0.0146 mmol), synthesized as described for compound HCB36 in Example 40 of PCT publication WO2022/235715, and let stir at r.t. until complete by LCMS. The solution was injected onto RP-FC and purified with a gradient of 0-70% MeCN in H₂O to furnish the title compound (1.0 mg, 9%). LCMS: C₄₃H₄₉N₇O₄ requires: 727.3. found: m/z=728.4 [M+H]⁺.

### Example 7

### rac-(3R)-3-[4-(4-{[(1-{1-[6-(2-hydroxyphenyl)pyridazin-4-yl]-4-phenylpiperidine-4-carbonyl}piperidin-4-yl)amino]methyl}piperidin-1-yl)phenyl]piperidine-2,6-dione

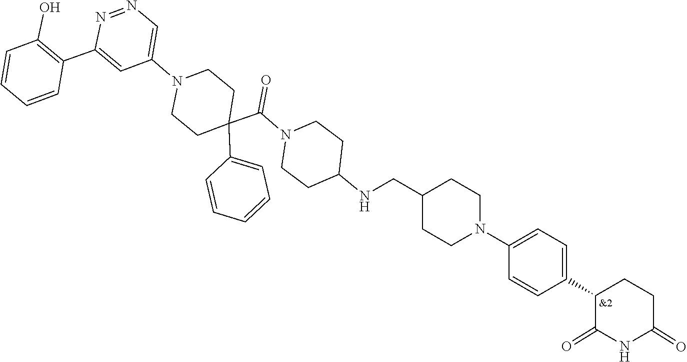 [SMILES: O=C1CC[C@@H](c2ccc(N3CCC(CNC4CCN(C(=O)C5(c6ccccc6)CCN(c6cnnc(-c7ccccc7O)c6)CC5)CC4)CC3)cc2)C(=O)N1]

### Step 1: Synthesis of rac-(R)-3-(4-(4-((piperidin-4-ylamino)methyl)piperidin-1-yl)phenyl)piperidine-2,6-dione

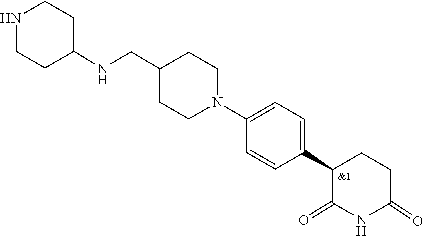 [SMILES: O=C1CC[C@H](c2ccc(N3CCC(CNC4CCNCC4)CC3)cc2)C(=O)N1]

rac-1-{4-[(3R)-2,6-dioxopiperidin-3-yl]phenyl}piperidine-4-carbaldehyde (100.00 mg, 0.3329 mmol), synthesized as described for compound HCB62 in Example 59 of PCT publication WO2022/235715, and tert-butyl 4-aminopiperidine-1-carboxylate (66.68 mg, 0.3329 mmol) were dissolved in DCE and N,N-diisopropylethylamine (297.61 μL, 226.18 mg, 1.7500 mmol) was added and the reaction stirred for 30 min. Added sodium triacetoxyborohydride (211.69 mg, 0.9988 mmol) and stirred overnight at room temperature. The reaction was concentrated and crude purified via RP-FC to yield tert-butyl rac-(R)-3-(4-(4-((piperidin-4-ylamino)methyl)piperidin-1-yl)phenyl)piperidine-2,6-dione (146 mg, 83%). The material was dissolved in 1:1 TFA:DCM, stirred for 1 h, then concentrated and purified by RP-FC to furnish the title compound, which was used crude without additional purification. LCMS: C₂₇H₄₀N₄O₄ requires: 384.5. found: m/z=385.6 [M+H]⁺.

### Step 2: Synthesis of the Title Compound

Stirred 1-[6-(2-hydroxyphenyl)pyridazin-4-yl]-4-phenylpiperidine-4-carboxylic acid (5.00 mg, 0.0133 mmol), [(dimethylamino)({[1,2,3]triazolo[4,5-b]pyridin-3-yloxy})methylidene]dimethylazanium; hexafluoro-lambda5-phosphanuide (10.13 mg, 0.0266 mmol), and N,N-diisopropylethylamine (9.30 μL, 6.89 mg, 0.0533 mmol) in DMF. Add rac-(3R)-3-(4-{4-[(piperidin-4-ylamino)methyl]piperidin-1-yl}phenyl)piperidine-2,6-dione (5.63 mg, 0.0146 mmol) and let stir at r.t. until complete by LCMS. The solution was injected onto RP-FC and purified with a gradient of 0-70% MeCN in H₂O to furnish the title compound (0.8 mg, 8%). LCMS: C₄₄H₅₁N₇O₄ requires: 741.4. found: m/z=742.4 [M+H]⁺.

### Example 8

### (3S)-3-(4-{4-[(6-{1-[6-(2-hydroxyphenyl)pyridazin-4-yl]-4-phenylpiperidine-4-carbonyl}-2,6-diazaspiro[3.3]heptan-2-yl)methyl]piperidin-1-yl}phenyl)piperidine-2,6-dione

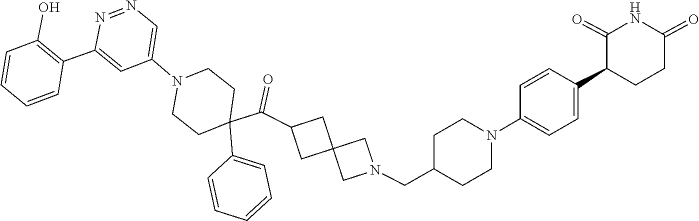 [SMILES: O=C1CC[C@@H](c2ccc(N3CCC(CN4CC5(CC(C(=O)C6(c7ccccc7)CCN(c7cnnc(-c8ccccc8O)c7)CC6)C5)C4)CC3)cc2)C(=O)N1]

### Step 1: Synthesis of (S)-3-(4-(4-((2,6-diazaspiro[3.3]heptan-2-yl)methyl)piperidin-1-yl)phenyl)piperidine-2,6-dione

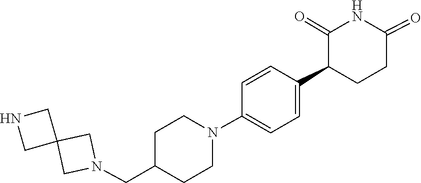 [SMILES: O=C1CC[C@@H](c2ccc(N3CCC(CN4CC5(CNC5)C4)CC3)cc2)C(=O)N1]

This intermediate was synthesized according to Step 1 of Example 7 using tert-butyl 2,6-diazaspiro[3.3]heptane-2-carboxylate. LCMS: C₂₂H₃₀N₄O₂ requires: 382.5. found: m/z=383.4 [M+H]⁺.

### Step 2: Synthesis of the Title Compound

Stirred 1-[6-(2-hydroxyphenyl)pyridazin-4-yl]-4-phenylpiperidine-4-carboxylic acid (30.00 mg, 0.0799 mmol), [(dimethylamino)({[1,2,3]triazolo[4,5-b]pyridin-3-yloxy})methylidene]dimethylazanium; hexafluoro-lambda5-phosphanuide (60.77 mg, 0.1598 mmol) and N,N-diisopropylethylamine (0.06 mL, 41.31 mg, 0.3196 mmol) in DMF. Add (3S)-3-[4-(4-{2,6-diazaspiro[3.3]heptan-2-ylmethyl}piperidin-1-yl)phenyl]piperidine-2,6-dione (33.62 mg, 0.0879 mmol) and let stir at r.t. until complete by LCMS. The solution was injected onto RP-FC and purified with a gradient of 0-70% MeCN in H₂O to furnish the title compound (10.1 mg, 16%). LCMS: C₄₄H₄₉N₇O₄ requires: 739.4. found: m/z=740.4 [M+H]⁺.

### Example 9

### (3S)-3-(4-{4-[(2-{1-[6-(2-hydroxyphenyl)pyridazin-4-yl]-4-phenylpiperidine-4-carbonyl}-2,6-diazaspiro[3.4]octan-6-yl)methyl]piperidin-1-yl}phenyl)piperidine-2,6-dione

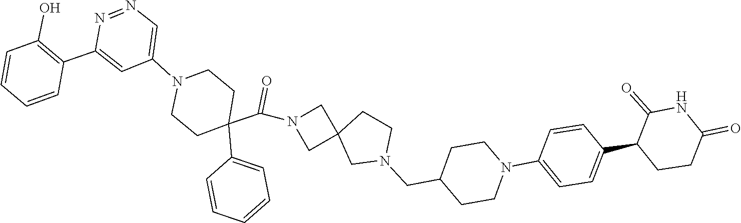 [SMILES: O=C1CC[C@@H](c2ccc(N3CCC(CN4CCC5(C4)CN(C(=O)C4(c6ccccc6)CCN(c6cnnc(-c7ccccc7O)c6)CC4)C5)CC3)cc2)C(=O)N1]

### Step 1: (S)-3-(4-(4-((2,6-diazaspiro[3.4]octan-6-yl)methyl)piperidin-1-yl)phenyl)piperidine-2,6-dione

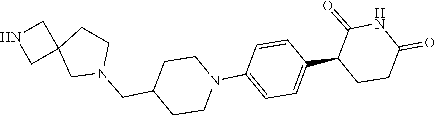 [SMILES: O=C1CC[C@@H](c2ccc(N3CCC(CN4CCC5(CNC5)C4)CC3)cc2)C(=O)N1]

This intermediate was synthesized according to Step 1 of Example 7 using tert-butyl 2,6-diazaspiro[3.4]octane-2-carboxylate. LCMS: C₂₃H₃₂N₄O₂ requires: 396.5. found: m/z=397.5 [M+H]⁺.

### Step 2: Synthesis of the Title Compound

Stir 1-[6-(2-hydroxyphenyl)pyridazin-4-yl]-4-phenylpiperidine-4-carboxylic acid (8.00 mg, 0.0213 mmol), [(dimethylamino)({[1,2,3]triazolo[4,5-b]pyridin-3-yloxy})methylidene]dimethylazanium; hexafluoro-lambda5-phosphanuide (16.20 mg, 0.0426 mmol), and N,N-diisopropylethylamine (14.89 μL, 11.02 mg, 0.0852 mmol) in DMF. Add (3S)-3-[4-(4-{2,6-diazaspiro[3.4]octan-6-ylmethyl}piperidin-1-yl)phenyl]piperidine-2,6-dione (9.29 mg, 0.0234 mmol) and let stir at r.t. until complete by LCMS. The solution was injected onto RP-FC and purified with a gradient of 0-70% MeCN in H₂O to furnish the title compound (3.4 mg, 21%). LCMS: C₄₅H₅₁N₇O₄ requires: 753.4. found: m/z=754.4[M+H]⁺.

### Example 10

### N-({1-[(1-{4-[(3S)-2,6-dioxopiperidin-3-yl]phenyl}piperidin-4-yl)methyl]piperidin-3-yl}methyl)-1-[6-(2-hydroxyphenyl)pyridazin-4-yl]-4-phenylpiperidine-4-carboxamide

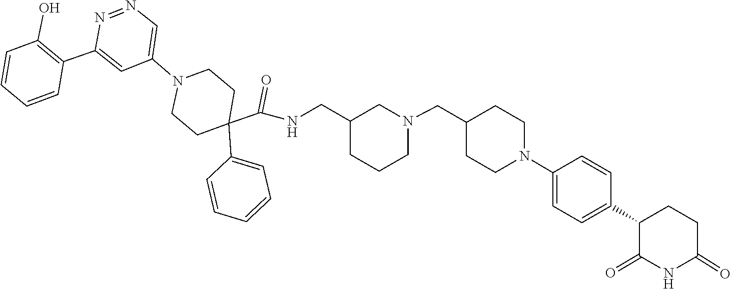 [SMILES: O=C1CC[C@@H](c2ccc(N3CCC(CN4CCCC(CNC(=O)C5(c6ccccc6)CCN(c6cnnc(-c7ccccc7O)c6)CC5)C4)CC3)cc2)C(=O)N1]

### Step 1: (3S)-3-(4-(4-((3-(aminomethyl)piperidin-1-yl)methyl)piperidin-1-yl)phenyl)piperidine-2,6-dione

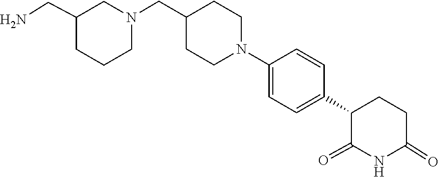 [SMILES: NCC1CCCN(CC2CCN(c3ccc([C@@H]4CCC(=O)NC4=O)cc3)CC2)C1]

This intermediate was synthesized according to Step 1 of Example 7 using tert-butyl (piperidin-3-ylmethyl)carbamate. LCMS: C₂₃H₃₄N₄O₂ requires: 398.5. found: m/z=399.5 [M+H]⁺.

### Step 2: Synthesis of the Title Compound

Stirred 1-[6-(2-hydroxyphenyl)pyridazin-4-yl]-4-phenylpiperidine-4-carboxylic acid (8.00 mg, 0.0213 mmol), [(dimethylamino)({[1,2,3]triazolo[4,5-b]pyridin-3-yloxy})methylidene]dimethylazanium; hexafluoro-lambda5-phosphanuide (16.20 mg, 0.0426 mmol), and N,N-diisopropylethylamine (14.89 μL, 11.02 mg, 0.0852 mmol) in DMF. Add (3S)-3-[4-(4-{[3-(aminomethyl)piperidin-1-yl]methyl}piperidin-1-yl)phenyl]piperidine-2,6-dione (9.34 mg, 0.0234 mmol) and let stir at r.t. until complete by LCMS. The solution was injected onto RP-FC and purified with a gradient of 0-70% MeCN in H₂O to furnish the title compound (0.8 mg, 5%). LCMS: C₄₅H₅₁N₇O₄ requires: 755.4. found: m/z=756.4 [M+H]⁺.

### Example 11

### rac-(3R)-3-[4-({1-[(1-{1-[6-(2-hydroxyphenyl)pyridazin-4-yl]-4-phenylpiperidine-4-carbonyl}piperidin-4-yl)methyl]piperidin-4-yl}methoxy)phenyl]piperidine-2,6-dione

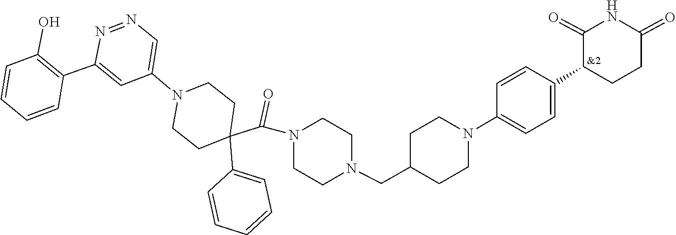 [SMILES: O=C1CC[C@H](c2ccc(N3CCC(CN4CCN(C(=O)C5(c6ccccc6)CCN(c6cnnc(-c7ccccc7O)c6)CC5)CC4)CC3)cc2)C(=O)N1]

### Step 1: Synthesis of rac-(R)-3-(4-(piperidin-4-ylmethoxy)phenyl)piperidine-2,6-dione

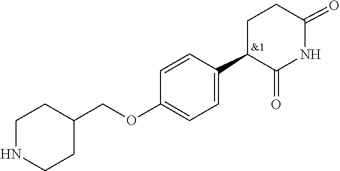 [SMILES: O=C1CC[C@H](c2ccc(OCC3CCNCC3)cc2)C(=O)N1]

To a 20 mL vial was added tert-butyl 4-{4-[2,6-bis(benzyloxy)pyridin-3-yl]phenoxymethyl}piperidine-1-carboxylate (277.00 mg, 0.4770 mmol), Pd/C (100.00 mg, mmol), EtOH (2.00 mL), and THF (2.00 mL). The reaction mixture was sparged with H₂ for 10 min, then stirred under H₂ atmosphere (balloon) for 16 h. The reaction mixture was then filtered through a pad of celite, then concentrated to furnish the Boc intermediate (162 mg, 84%) which was dissolved in 1:1 TFA:DCM for 1 h then concentrated and lyophilized to yield the title compound. LCMS: C₁₇H₂₂N₂O₃ requires: 302.4. found: m/z=303.3 [M+H]⁺.

### Step 2: Synthesis of rac-(R)-3-(4-((1-(piperidin-4-ylmethyl)piperidin-4-yl)methoxy)phenyl)piperidine-2,6-dione

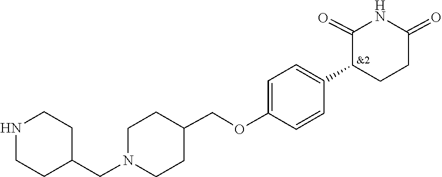 [SMILES: O=C1CC[C@H](c2ccc(OCC3CCN(CC4CCNCC4)CC3)cc2)C(=O)N1]

rac-(R)-3-(4-(piperidin-4-ylmethoxy)phenyl)piperidine-2,6-dione and tert-butyl 4-formylpiperidine-1-carboxylate were dissolved in dry DCE. N,N-diisopropylethylamine (72.20 μL, 53.43 mg, 0.4134 mmol) was added and reaction stirred for 30 min. Added sodium triacetoxyborohydride (52.57 mg, 0.2480 mmol). Stirred overnight at r.t. then and purified via RP-FC to furnish the Boc protected intermediate (30 mg, 66%). This material was dissolved in 1:1 TFA:DCM, stirred for 1 h at room temperature, then concentrated and used in the next step without additional purification. LCMS: C₂₃H₃₃N₃O₃ requires: 399.3. found: m/z=400.4 [M+H]⁺.

### Step 3: Synthesis of the title compound

Stir 1-[6-(2-hydroxyphenyl)pyridazin-4-yl]-4-phenylpiperidine-4-carboxylic acid (15.00 mg, 0.0400 mmol), [(dimethylamino)({[1,2,3]triazolo[4,5-b]pyridin-3-yloxy})methylidene]dimethylazanium; hexafluoro-lambda5-phosphanuide (30.38 mg, 0.0799 mmol), and N,N-diisopropylethylamine (27.91 μL, 20.66 mg, 0.1598 mmol) in DMF. Add rac-(3R)-3-(4-{[1-(piperidin-4-ylmethyl)piperidin-4-yl]methoxy}phenyl)piperidine-2,6-dione (17.56 mg, 0.0439 mmol) and let stir at r.t. until complete by LCMS. The solution was injected onto RP-FC and purified with a gradient of 0-70% MeCN in H₂O to furnish the title compound (1.2 mg, 4%). LCMS: C₄₅H₅₂N₆O₅ requires: 756.4. found: m/z=757.4 [M+H]⁺.

### Example 12

### rac-(3R)-3-[4-({1-[(1-{1-[6-(2-hydroxyphenyl)pyridazin-4-yl]-4-phenylpiperidine-4-carbonyl}piperidin-4-yl)methyl]piperidin-4-yl}oxy)phenyl]piperidine-2,6-dione

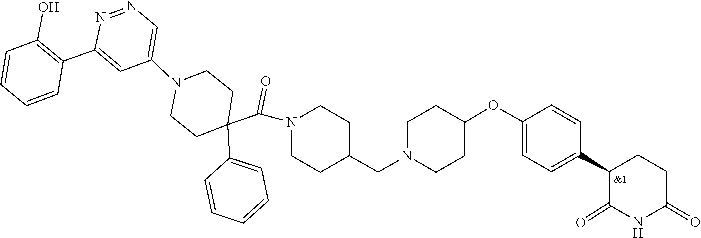 [SMILES: O=C1CC[C@H](c2ccc(OC3CCN(CC4CCN(C(=O)C5(c6ccccc6)CCN(c6cnnc(-c7ccccc7O)c6)CC5)CC4)CC3)cc2)C(=O)N1]

### Step 1: Synthesis of rac-(R)-3-(4-((1-(piperidin-4-ylmethyl)piperidin-4-yl)oxy)phenyl)piperidine-2,6-dione

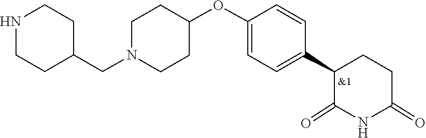 [SMILES: O=C1CC[C@H](c2ccc(OC3CCN(CC4CCNCC4)CC3)cc2)C(=O)N1]

rac-(3R)-3-[4-(piperidin-4-yloxy)phenyl]piperidine-2,6-dione (25.00 mg, 0.0827 mmol) and tert-butyl 4-formylpiperidine-1-carboxylate (17.63 mg, 0.0827 mmol) were dissolved in dry DCE. N,N-diisopropylethylamine (72.20 μL, 53.43 mg, 0.4134 mmol) was added and reaction stirred for 30 min. Added sodium triacetoxyborohydride (52.57 mg, 0.2480 mmol). Stirred overnight at r.t. then concentrated and purified via RP-FC to yield the Boc intermediate (30 mg, 69%) which was then dissolved in 1:1 TFA:DCM and stirred for 1 h, then concentrated and lyophilized. LCMS: C₂₇H₃₉N₃O₅ requires: 485.6. found: m/z=486.6 [M+H]⁺.

### Step 2: Synthesis of the Title Compound

Stir 1-[6-(2-hydroxyphenyl)pyridazin-4-yl]-4-phenylpiperidine-4-carboxylic acid (30.00 mg, 0.0799 mmol), [(dimethylamino)({[1,2,3]triazolo[4,5-b]pyridin-3-yloxy})methylidene]dimethylazanium; hexafluoro-lambda5-phosphanuide (60.77 mg, 0.1598 mmol), and N,N-diisopropylethylamine (55.83 μL, 41.31 mg, 0.3196 mmol) in DMF. Add rac-(3R)-3-(4-{[1-(piperidin-4-ylmethyl)piperidin-4-yl]oxy}phenyl)piperidine-2,6-dione (33.89 mg, 0.0879 mmol) and let stir at r.t. until complete by LCMS. The solution was injected onto RP-FC and purified with a gradient of 0-70% MeCN in H₂O to furnish the title compound (3.4 mg, 6%). LCMS: C₄₄H₅₀N₆O₅ requires: 742.4. found: m/z=743.4 [M+H]⁺.

### Example 13

### rac-(3R)-3-(4-{[1-(1-{1-[6-(2-hydroxyphenyl)pyridazin-4-yl]-4-phenylpiperidine-4-carbonyl}piperidine-4-carbonyl)piperidin-4-yl]oxy}phenyl)piperidine-2,6-dione

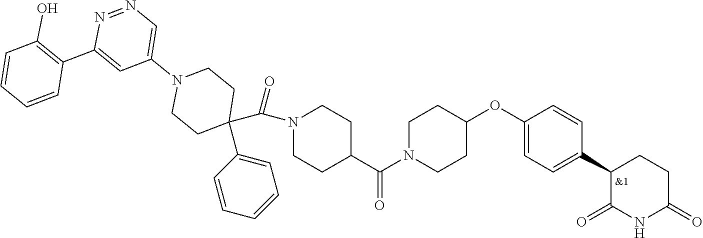 [SMILES: O=C1CC[C@H](c2ccc(OC3CCN(C(=O)C4CCN(C(=O)C5(c6ccccc6)CCN(c6cnnc(-c7ccccc7O)c6)CC5)CC4)CC3)cc2)C(=O)N1]

### Step 1: Synthesis of rac-(R)-3-(4-((1-(piperidine-4-carbonyl)piperidin-4-yl)oxy)phenyl)piperidine-2,6-dione

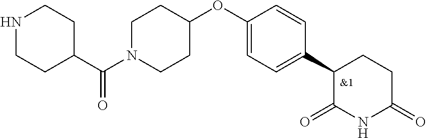 [SMILES: O=C1CC[C@H](c2ccc(OC3CCN(C(=O)C4CCNCC4)CC3)cc2)C(=O)N1]

Stir rac-(3R)-3-[4-(piperidin-4-yloxy)phenyl]piperidine-2,6-dione (45.00 mg, 0.1561 mmol), [(dimethylamino)({[1,2,3]triazolo[4,5-b]pyridin-3-yloxy})methylidene]dimethylazanium; hexafluoro-lambda5-phosphanuide (118.68 mg, 0.3121 mmol), and N,N-diisopropylethylamine (80.68 mg, 0.6242 mmol) in DMF. Add 1-(tert-butoxycarbonyl)piperidine-4-carboxylic acid (35.78 mg, 0.1561 mmol) and let stir at r.t. until complete by LCMS. Inject directly and purify using RP FC to yield the Boc protected intermediate (28 mg, 36%). This material was then dissolved in 1:1 TFA:DCM and stirred 1 h at r.t. then concentrated and used in the next step. LCMS: C₂₂H₂₉N₃O₄ requires: 399.5. found: m/z=400.5 [M+H]⁺.

### Step 2: Synthesis of the Title Compound

Stir 1-[6-(2-hydroxyphenyl)pyridazin-4-yl]-4-phenylpiperidine-4-carboxylic acid (22.00 mg, 0.0586 mmol), [(dimethylamino)({[1,2,3]triazolo[4,5-b]pyridin-3-yloxy})methylidene]dimethylazanium; hexafluoro-lambda5-phosphanuide (44.56 mg, 0.1172 mmol), and N,N-diisopropylethylamine (40.94 μL, 30.30 mg, 0.2344 mmol) in DMF. Add rac-(3R)-3-(4-{[1-(piperidine-4-carbonyl)piperidin-4-yl]oxy}phenyl)piperidine-2,6-dione (25.75 mg, 0.0645 mmol) and let stir at r.t. until complete by LCMS. The solution was injected onto RP-FC and purified with a gradient of 0-70% MeCN in H₂O to furnish the title compound (7.2 mg, 15%). LCMS: C₄₄H₄₈N₆O₅ requires: 756.4. found: m/z=757.2 [M+H]⁺.

### Example 14

### rac-(3R)-3-(4-{[1-(1-{1-[6-(2-hydroxyphenyl)pyridazin-4-yl]-4-phenylpiperidine-4-carbonyl}piperidine-4-carbonyl)piperidin-4-yl]methoxy}phenyl)piperidine-2,6-dione

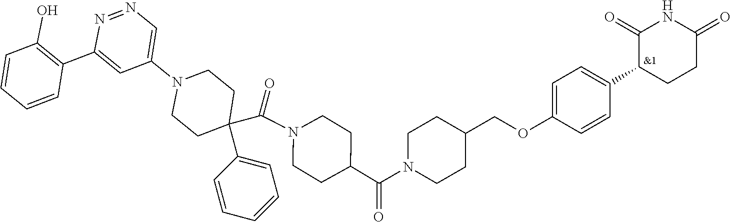 [SMILES: O=C1CC[C@H](c2ccc(OCC3CCN(C(=O)C4CCN(C(=O)C5(c6ccccc6)CCN(c6cnnc(-c7ccccc7O)c6)CC5)CC4)CC3)cc2)C(=O)N1]

### Step 1: Synthesis of rac-(R)-3-(4-((1-(piperidine-4-carbonyl)piperidin-4-yl)methoxy)phenyl)piperidine-2,6-dione

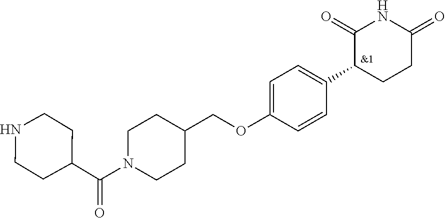 [SMILES: O=C1CC[C@H](c2ccc(OCC3CCN(C(=O)C4CCNCC4)CC3)cc2)C(=O)N1]

Stir rac-(3R)-3-[4-(piperidin-4-ylmethoxy)phenyl]piperidine-2,6-dione (45.00 mg, 0.1488 mmol), [(dimethylamino)({[1,2,3]triazolo[4,5-b]pyridin-3-yloxy})methylidene]dimethylazanium; hexafluoro-lambda5-phosphanuide (113.17 mg, 0.2976 mmol), and N,N-diisopropylethylamine (103.97 μL, 76.94 mg, 0.5953 mmol) in DMF. Add 1-(tert-butoxycarbonyl)piperidine-4-carboxylic acid (34.12 mg, 0.1488 mmol) and let stir at r.t. until complete by LCMS. Direct inject to RP-FC and isolate Boc protected intermediate (45 mg, 58%). Concentrate and dissolve in 1:1 TFA:DCM and stir for 1 h, then concentrate and lyophilize to use in next step. LCMS: C₂₃H₃₁N₃O₄ requires: 413.6, found: m/z=414.6 [M+H]⁺.

### Step 2: Synthesis of the Title Compound

Stirred 1-[6-(2-hydroxyphenyl)pyridazin-4-yl]-4-phenylpiperidine-4-carboxylic acid (22.00 mg, 0.0586 mmol), [(dimethylamino)({[1,2,3]triazolo[4,5-b]pyridin-3-yloxy})methylidene]dimethylazanium; hexafluoro-lambda5-phosphanuide (44.56 mg, 0.1172 mmol), and N,N-diisopropylethylamine (40.94 μL, 30.30 mg, 0.2344 mmol) in DMF. Add rac-(3R)-3-(4-{[1-(piperidine-4-carbonyl)piperidin-4-yl]methoxy}phenyl)piperidine-2,6-dione (26.66 mg, 0.0645 mmol) and let stir at r.t. until complete by LCMS. The solution was injected onto RP-FC and purified with a gradient of 0-70% MeCN in H₂O to furnish the title compound (2.4 mg, 5%). LCMS: C₄₅H₅₀N₆O₄ requires: 770.4. found: m/z=771.4 [M+H]⁺.

### Example 15

### (3R)-3-(4-{4-[(6-{1-[6-(2-hydroxyphenyl)pyridazin-4-yl]-4-phenylpiperidine-4-carbonyl}-2,6-diazaspiro[3.3]heptan-2-yl)methyl]piperidin-1-yl}phenyl)piperidine-2,6-dione

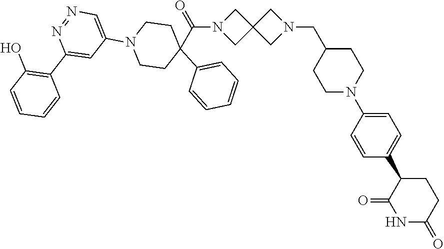 [SMILES: O=C1CC[C@H](c2ccc(N3CCC(CN4CC5(C4)CN(C(=O)C4(c6ccccc6)CCN(c6cnnc(-c7ccccc7O)c6)CC4)C5)CC3)cc2)C(=O)N1]

### Step 1: Synthesis of (1-(6-(2-hydroxyphenyl)pyridazin-4-yl)-4-phenylpiperidin-4-yl)(2,6-diazaspiro[3.3]heptan-2-yl)methanone

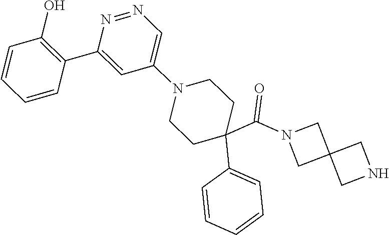 [SMILES: O=C(N1CC2(CNC2)C1)C1(c2ccccc2)CCN(c2cnnc(-c3ccccc3O)c2)CC1]

Stir 1-[6-(2-hydroxyphenyl)pyridazin-4-yl]-4-phenylpiperidine-4-carboxylic acid (10.00 mg, 0.0266 mmol), [(dimethylamino)({[1,2,3]triazolo[4,5-b]pyridin-3-yloxy})methylidene]dimethylazanium; hexafluoro-lambda5-phosphanuide (20.26 mg, 0.0533 mmol), and N,N-diisopropylethylamine (18.61 μL, 13.77 mg, 0.1065 mmol) in DMF. Add tert-butyl 2,6-diazaspiro[3.3]heptane-2-carboxylate (5.81 mg, 0.0293 mmol) and let stir at r.t. until complete by LCMS. Inject directly and purify via RP-FC to yield the Boc intermediate (6.2 mg, 37%). This material was dissolved in 1:1 TFA:DCM and stirred 1 h at r.t. then concentrated, lyophilized and used without further purification. LCMS: C₂₇H₂₉N₅O₂ requires: 455.3. found: m/z=456.5 [M+H]⁺.

### Step 2: Synthesis of the Title Compound

To a scintillation vial was added 2-[5-(4-{2,6-diazaspiro[3.3]heptane-2-carbonyl}-4-phenylpiperidin-1-yl)pyridazin-3-yl]phenol (42.00 mg, 0.0922 mmol), triethylamine (63.04 μL, 46.65 mg, 0.4610 mmol), 1-{4-[(3R)-2,6-dioxopiperidin-3-yl]phenyl}piperidine-4-carbaldehyde (33.23 mg, 0.1106 mmol) and DCE (3.00 mL) Stir at r.t. and add sodium triacetoxyborohydride (68.39 mg, 0.3227 mmol). Stir until complete consumption of starting material by MS. This reaction was complete and clean after 90 minutes. Quench the reaction by adding bicarbonate solution and extract with EtOAc. Dry the organic layer with brine and then over Na₂SO₄. Concentrated and the solution was injected onto RP-FC and purified with a gradient of 0-70% MeCN in H₂O to furnish the title compound (27.4 mg, 40%). LCMS: C₄₄H₄₉N₇O₄ requires: 739.4. found: m/z=740.4 [M+H]⁺.

¹H NMR (500 MHz, DMSO) δ 10.69 (d, J=2.5 Hz, 1H), 9.57 (d, J=0.9 Hz, 1H), 7.35 (t, J=7.2 Hz, 1H), 7.30-7.23 (m, 1H), 7.00-6.88 (m, 5H), 6.90-6.75 (m, 5H), 5.83 (d, J=7.6 Hz, 1H), 4.11 (dd, J=7.5, 5.9 Hz, 1H), 3.68-3.54 (m, 3H), 3.60 (s, 3H), 3.57-3.49 (m, 1H), 3.49 (t, J=4.1 Hz, 1H), 3.10 (d, J=4.8 Hz, 1H), 2.92 (d, J=13.3 Hz, 1H), 2.74 (ddd, J=12.4, 10.9, 2.9 Hz, 2H), 2.61-2.45 (m, 5H), 2.43-2.37 (m, 2H), 2.37 (tt, J=4.7, 2.3 Hz, 1H), 2.13-1.99 (m, 2H), 1.98-1.89 (m, 3H), 1.89-1.81 (m, 2H), 1.72-1.65 (m, 2H), 1.59-1.38 (m, 2H), 1.26 (dtdd, J=16.3, 12.6, 7.9, 3.6 Hz, 2H).

### Example 16

### (3R)-3-(4-{4-[(6-{1-[6-(2-hydroxyphenyl)pyridazin-4-yl]-4-phenylpiperidine-4-carbonyl}-2,6-diazaspiro[3.4]octan-2-yl)methyl]piperidin-1-yl}phenyl)piperidine-2,6-dione

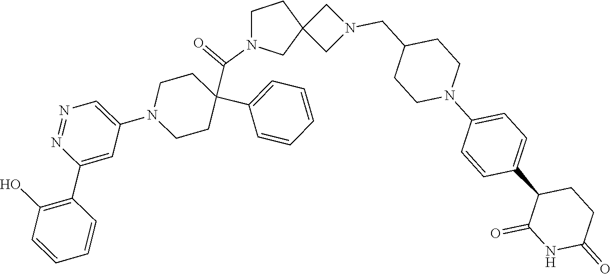 [SMILES: O=C1CC[C@H](c2ccc(N3CCC(CN4CC5(CCN(C(=O)C6(c7ccccc7)CCN(c7cnnc(-c8ccccc8O)c7)CC6)C5)C4)CC3)cc2)C(=O)N1]

### Step 1: Synthesis of (1-(6-(2-hydroxyphenyl)pyridazin-4-yl)-4-phenylpiperidin-4-yl)(2,6-diazaspiro[3.4]octan-6-yl)methanone

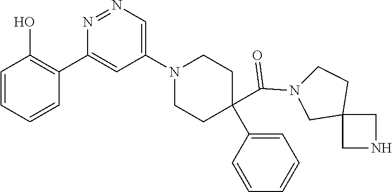 [SMILES: O=C(N1CCC2(CNC2)C1)C1(c2ccccc2)CCN(c2cnnc(-c3ccccc3O)c2)CC1]

Stir 1-[6-(2-hydroxyphenyl)pyridazin-4-yl]-4-phenylpiperidine-4-carboxylic acid (99.00 mg, 0.2637 mmol), [(dimethylamino)({[1,2,3]triazolo[4,5-b]pyridin-3-yloxy})methylidene]dimethylazanium; hexafluoro-lambda5-phosphanuide (250.67 mg, 0.6592 mmol), and N,N-diisopropylethylamine (230.29 μL, 170.41 mg, 1.3185 mmol) in DMF. Add tert-butyl 2,6-diazaspiro[3.4]octane-2-carboxylate (75.57 mg, 0.3560 mmol) and let stir at r.t. until complete by LCMS. Inject and purify directly via RP-FC to furnish the Boc intermediate (170 mg, 99%). Dissolve this material in 1:1 TFA:DCM and stir for 1 h at r.t. then concentrate and lyophilize to use in the next step. LCMS: C₂₈H₃₁N₅O₂ requires: 469.3. found: m/z=470.5 [M+H]⁺.

### Step 2: Synthesis of the Title Compound

To a scintillation vial was added 2-[5-(4-{2,6-diazaspiro[3.4]octane-6-carbonyl}-4-phenylpiperidin-1-yl)pyridazin-3-yl]phenol (42.00 mg, 0.0922 mmol), triethylamine (63.04 μL, 46.65 mg, 0.4610 mmol), 1-{4-[(3R)-2,6-dioxopiperidin-3-yl]phenyl}piperidine-4-carbaldehyde (33.23 mg, 0.1106 mmol) and DCE (3.00 mL) Stir at r.t. and add sodium triacetoxyborohydride (68.39 mg, 0.3227 mmol). Stir until complete consumption of starting material by MS. This reaction was complete and clean after 90 minutes. Quench the reaction by adding bicarbonate solution and extract with EtOAc. Dry the organic layer with brine and then over Na₂SO₄. Concentrated and the solution was injected onto RP-FC and purified with a gradient of 0-70% MeCN in H₂O to furnish the title compound (19.8 mg, 29%). LCMS: C₄₅H₅₁N₇O₄ requires: 753.4. found: m/z=754.4 [M+H]⁺.

¹H NMR (500 MHz, DMSO) δ 10.70 (s, 1H), 8.86 (d, J=2.8 Hz, 1H), 8.04 (dd, J=8.5, 1.7 Hz, 1H), 7.46 (d, J=3.2 Hz, 1H), 7.32 (d, J=7.5 Hz, 2H), 7.29-7.19 (m, 4H), 6.96 (d, J=8.2 Hz, 2H), 6.88-6.78 (m, 4H), 5.69 (s, 1H), 4.05 (d, J=14.5 Hz, 2H), 3.64 (dd, J=11.0, 4.9 Hz, 1H), 3.54 (d, J=12.0 Hz, 2H), 3.43 (s, 1H), 3.30 (q, J=9.0 Hz, 3H), 3.02 (s, 1H), 2.95 (s, 1H), 2.79 (s, 1H), 2.68-2.64 (m, 1H), 2.61-2.51 (m, 1H), 2.49 (s, 2H), 2.47 (d, J=2.2 Hz, 2H), 2.39 (dt, J=17.2, 4.6 Hz, 5H), 2.20 (s, 1H), 2.08-1.99 (m, 2H), 1.97-1.89 (m, 3H), 1.69 (t, J=7.3 Hz, 1H), 1.62 (s, 1H), 1.50 (d, J=12.3 Hz, 1H), 1.10 (s, 2H), 1.00 (dd, J=15.5, 5.1 Hz, 2H); LCMS: C₄₅H₅₁N₇O₄ requires: 753.4. found: m/z=754.8 [M+H]⁺.

### Example 17

### (3R)-3-(4-{4-[(2-{1-[6-(2-hydroxyphenyl)pyridazin-4-yl]-4-phenylpiperidine-4-carbonyl}-2,6-diazaspiro[3.4]octan-6-yl)methyl]piperidin-1-yl}phenyl)piperidine-2,6-dione

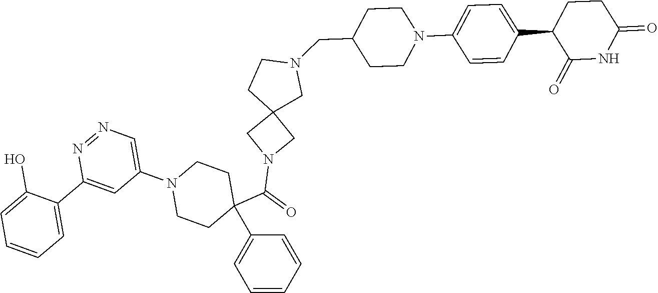 [SMILES: O=C1CC[C@H](c2ccc(N3CCC(CN4CCC5(C4)CN(C(=O)C4(c6ccccc6)CCN(c6cnnc(-c7ccccc7O)c6)CC4)C5)CC3)cc2)C(=O)N1]

### Step 1: Synthesis of (1-(6-(2-hydroxyphenyl)pyridazin-4-yl)-4-phenylpiperidin-4-yl)(2,6-diazaspiro[3.4]octan-2-yl)methanone

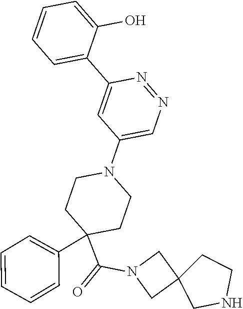 [SMILES: O=C(N1CC2(CCNC2)C1)C1(c2ccccc2)CCN(c2cnnc(-c3ccccc3O)c2)CC1]

Stir 1-[6-(2-hydroxyphenyl)pyridazin-4-yl]-4-phenylpiperidine-4-carboxylic acid (99.00 mg, 0.2637 mmol), [(dimethylamino)({[1,2,3]triazolo[4,5-b]pyridin-3-yloxy})methylidene]dimethylazanium; hexafluoro-lambda5-phosphanuide (250.67 mg, 0.6592 mmol), and N,N-diisopropylethylamine (230.29 μL, 170.41 mg, 1.3185 mmol) in DMF. Add tert-butyl 2,6-diazaspiro[3.4]octane-6-carboxylate (75.57 mg, 0.3560 mmol) and let stir at r.t. until complete by LCMS. Inject and purify directly via RP-FC to furnish the Boc intermediate (170 mg, 99%). Dissolve this material in 1:1 TFA:DCM and stir for 1 h at r.t. then concentrate and lyophilize to use in the next step. LCMS: C₂₈H₃₁N₅O₂ requires: 469.3. found: m/z=470.5 [M+H]⁺.

### Step 2: Synthesis of the Title Compound

To a scintillation vial was added 2-[5-(4-{2,6-diazaspiro[3.4]octane-2-carbonyl}-4-phenylpiperidin-1-yl)pyridazin-3-yl]phenol (42.00 mg, 0.0922 mmol), triethylamine (63.04 μL, 46.65 mg, 0.4610 mmol), 1-{4-[(3R)-2,6-dioxopiperidin-3-yl]phenyl}piperidine-4-carbaldehyde (33.23 mg, 0.1106 mmol) and DCE (3.00 mL) Stir at r.t. and add sodium triacetoxyborohydride (68.39 mg, 0.3227 mmol). Stir until complete consumption of starting material by MS. This reaction was complete and clean after 90 minutes. Quench the reaction by adding bicarbonate solution and extract with EtOAc. Dry the organic layer with brine and then over Na₂SO₄. Concentrated and the solution was injected onto RP-FC and purified with a gradient of 0-70% MeCN in H₂O to furnish the title compound (24.0 mg, 31%). LCMS: C₄₅H₅₁N₇O₄ requires: 753.4. found: m/z=754.4 [M+H]⁺.

¹H NMR (500 MHz, DMSO) δ 10.70 (d, J=3.6 Hz, 1H), 9.57 (s, 1H), 8.89 (d, J=2.9 Hz, 1H), 8.02 (dd, J=8.4, 1.7 Hz, 1H), 7.48 (d, J=3.0 Hz, 1H), 7.35 (dd, J=8.4, 7.0 Hz, 1H), 7.30-7.22 (m, 3H), 7.00-6.93 (m, 3H), 6.93-6.81 (m, 3H), 6.84-6.77 (m, 2H), 4.16-3.99 (m, 3H), 3.87 (s, 1H), 3.78 (s, 1H), 3.63 (ddd, J=16.4, 11.5, 4.7 Hz, 3H), 3.59 (d, J=4.8 Hz, 1H), 3.50 (dt, J=12.6, 4.1 Hz, 1H), 3.31 (t, J=11.7 Hz, 1H), 3.17 (s, 1H), 3.10 (d, J=4.7 Hz, 2H), 2.73 (ddd, J=12.3, 10.9, 2.9 Hz, 1H), 2.61-2.45 (m, 4H), 2.43-2.34 (m, 3H), 2.29 (d, J=15.9 Hz, 2H), 2.05 (tdd, J=13.7, 8.2, 3.2 Hz, 2H), 1.98-1.81 (m, 2H), 1.68 (dd, J=8.6, 3.8 Hz, 1H), 1.66 (s, 2H), 1.57-1.38 (m, 1H), 1.33-1.18 (m, 1H), 1.17 (s, 2H), 1.15 (d, J=11.2 Hz, 2H).

### Example 18

### rac-(3R)-3-(4-{4-[2-(1-{1-[6-(2-hydroxyphenyl)pyridazin-4-yl]-4-phenylpiperidine-4-carbonyl}piperidin-4-yl)acetyl]piperazin-1-yl}phenyl)piperidine-2,6-dione

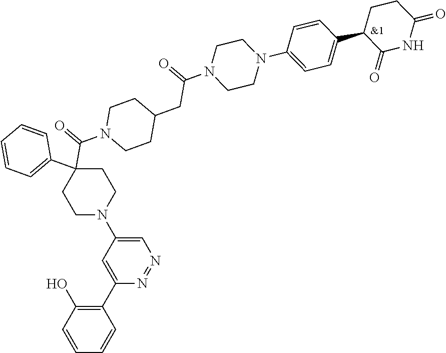 [SMILES: O=C1CC[C@H](c2ccc(N3CCN(C(=O)CC4CCN(C(=O)C5(c6ccccc6)CCN(c6cnnc(-c7ccccc7O)c6)CC5)CC4)CC3)cc2)C(=O)N1]

### Step 1: Synthesis of (1-{1-[6-(2-hydroxyphenyl)pyridazin-4-yl]-4-phenylpiperidine-4-carbonyl}piperidin-4-yl)acetic acid

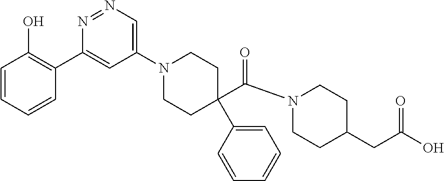 [SMILES: O=C(O)CC1CCN(C(=O)C2(c3ccccc3)CCN(c3cnnc(-c4ccccc4O)c3)CC2)CC1]

Stir 1-[6-(2-hydroxyphenyl)pyridazin-4-yl]-4-phenylpiperidine-4-carboxylic acid (70.00 mg, 0.1865 mmol), [(dimethylamino)({[1,2,3]triazolo[4,5-b]pyridin-3-yloxy})methylidene]dimethylazanium; hexafluoro-lambda5-phosphanuide (177.24 mg, 0.4661 mmol), and N,N-diisopropylethylamine (162.83 μL, 120.49 mg, 0.9323 mmol) in DMF. Add tert-butyl 2-(piperidin-4-yl)acetate (49.09 μL, 46.63 mg, 0.2517 mmol) and let stir at r.t. until complete by LCMS. Inject directly on RP-FC and purify to isolate the t-Bu protected intermediate (80 mg, 69%). Dissolve this material in 1:1 TFA:DCM and stir for 1 h at r.t. LCMS: C₂₉H₃₂N₄O₄ requires: 500.6. found: m/z=501.4 [M+H]⁺.

### Step 2: Synthesis of the Title Compound

Stirred (1-{1-[6-(2-hydroxyphenyl)pyridazin-4-yl]-4-phenylpiperidine-4-carbonyl}piperidin-4-yl)acetic acid (10.00 mg, 0.0200 mmol), [(dimethylamino)({[1,2,3]triazolo[4,5-b]pyridin-3-yloxy})methylidene]dimethylazanium; hexafluoro-lambda5-phosphanuide (18.99 mg, 0.0499 mmol), and N,N-diisopropylethylamine (17.44 μL, 12.91 mg, 0.0999 mmol) in DMF. Added rac-(3R)-3-[4-(piperazin-1-yl)phenyl]piperidine-2,6-dione (6.55 mg, 0.0240 mmol), synthesized as described for compound 5 in PCT publication WO2023/018238, and let stir at r.t. until complete by LCMS. The solution was injected onto RP-FC and purified with a gradient of 0-70% MeCN in H₂O to furnish the title compound (4.3 mg, 29%). LCMS: C₄₄H₄₉N₇O₅ requires: 755.4. found: m/z=756.7 [M+H]⁺.

### Example 19

### rac-(3R)-3-(6-{4-fluoro-4-[(6-{1-[6-(2-hydroxyphenyl)pyridazin-4-yl]-4-phenylpiperidine-4-carbonyl}-2,6-diazaspiro[3.3]heptan-2-yl)methyl]piperidin-1-yl}pyridin-3-yl)piperidine-2,6-dione

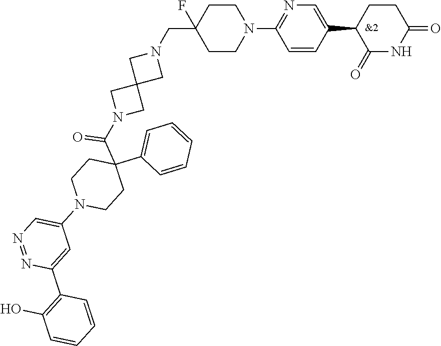 [SMILES: O=C1CC[C@H](c2ccc(N3CCC(F)(CN4CC5(C4)CN(C(=O)C4(c6ccccc6)CCN(c6cnnc(-c7ccccc7O)c6)CC4)C5)CC3)nc2)C(=O)N1]

### Step 1: Synthesis of (4-fluoro-1-(5-iodopyridin-2-yl)piperidin-4-yl)methanol

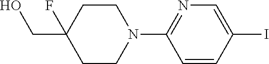 [SMILES: OCC1(F)CCN(c2ccc(I)cn2)CC1]

To a solution of 2-bromo-5-iodopyridine (5.92 g, 26.5 mmol, 1.00 eq) in DMF (45.0 mL) was added (4-fluoropiperidin-4-yl)methanol (4.50 g, 26.5 mmol, 1.00 eq, HCl) and K₂CO₃ (12.8 g, 92.9 mmol, 3.50 eq) at 20° C. The mixture was heated to 90° C. and stirred at 90° C. for 16 hrs. The reaction mixture was poured into water (225 mL), stirred at 20° C. for 5 mins, filtered, and the filter cake was concentrated under vacuum. The crude product was triturated with petroleum ether:ethyl acetate=3:1 (25.0 mL) at 25° C. for 2 hrs, filtered, and the filter cake was concentrated under vacuum. The filtrate was concentrated under vacuum, then purified by silica gel chromatography (SiO₂, petroleum ether:ethyl acetate=12:1 to 5:1). (4-fluoro-1-(5-iodopyridin-2-yl)piperidin-4-yl)methanol (4.04 g, crude) was obtained as a white solid. LCMS: C₁₁H₁₄FIN₂O requires: 336.0, found: m/z=337.0 [M+H]⁺. ¹H NMR (400 MHz, MeOD) δ 8.22 (d, J=2.0 Hz, 1H), 7.73 (dd, J=2.4, 9.2 Hz, 1H), 6.73 (d, J=8.8 Hz, 1H), 4.10-4.06 (m, 2H), 3.56 (d, J=19.6 Hz, 2H), 3.25-3.18 (m, 2H), 1.89-1.84 (m, 2H), 1.77-1.64 (m, 2H).

### Step 2: Synthesis of (1-(2′,6′-bis(benzyloxy)-[3,3′-bipyridin]-6-yl)-4-fluoropiperidin-4-yl)methanol

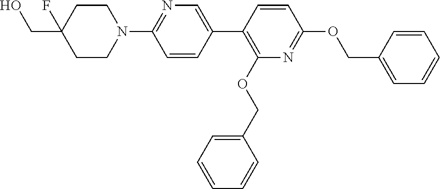 [SMILES: OCC1(F)CCN(c2ccc(-c3ccc(OCc4ccccc4)nc3OCc3ccccc3)cn2)CC1]

To a solution of (4-fluoro-1-(5-iodopyridin-2-yl)piperidin-4-yl)methanol (3.60 g, 10.7 mmol, 1.10 eq) and 2,6-bis(benzyloxy)-4-(4,4,5,5-tetramethyl-1,3,2-dioxaborolan-2-yl)pyridine (4.06 g, 9.74 mmol, 1.00 eq) in dioxane (32.4 mL) and H₂O (3.60 mL) was added XPhos Pd G3 (824 mg, 974 μmol, 0.10 eq). K₃PO₄ (5.17 g, 24.3 mmol, 2.50 eq) was added to the mixture at 20° C. under N₂, then the mixture was degassed and purged with N₂ 3 times, and then the mixture was stirred at 90° C. for 12 hrs under N₂ atmosphere. The reaction mixture was concentrated under vacuum. The residue was purified by column chromatography (SiO₂, petroleum ether:ethyl acetate=3:1 to 1:1). (1-(2′,6′-bis(benzyloxy)-[3,3′-bipyridin]-6-yl)-4-fluoropiperidin-4-yl)methanol (3.64 g, 7.25 mmol, 74.5% yield, 99.6% purity) was obtained as a white solid. LCMS: C₃₀H₃₀FN₃O₃ requires: 499.2. found: m/z=500.2 [M+H]⁺. ¹H NMR: (400 MHz, MeOD) δ 8.27 (d, J=2.4 Hz, 1H), 7.77 (dd, J=12, 11.2 Hz, 1H), 7.62 (d, J=8.0 Hz, 1H), 7.40-7.38 (m, 2H), 7.35-7.23 (m, 8H), 6.85 (d, J=8.8 Hz, 1H), 6.47 (d, J=8 Hz, 1H), 5.37 (s, 2H), 5.34 (s, 2H), 4.09-4.06 (m, 2H), 3.56 (d, J=20 Hz, 2H), 3.27-3.20 (m, 2H), 1.91-1.85 (m, 2H), 1.80-1.63 (m, 2H).

### Step 3: Synthesis of rac-(R)-3-(6-(4-fluoro-4-(hydroxymethyl)piperidin-1-yl)pyridin-3-yl)piperidine-2,6-dione

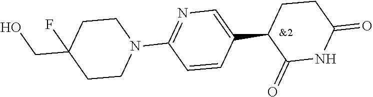 [SMILES: O=C1CC[C@H](c2ccc(N3CCC(F)(CO)CC3)nc2)C(=O)N1]

To a solution of (1-(2′,6′-bis(benzyloxy)-[3,3′-bipyridin]-6-yl)-4-fluoropiperidin-4-yl)methanol (2.60 g, 5.20 mmol, 1.00 eq) in THF (13.0 mL) and EtOH (13.0 mL) was added AcOH (313 mg, 5.20 mmol, 298 μL, 1.00 eq) and Pd/C (1.30 g, 10.0% purity) under N₂ atmosphere at 20° C. The suspension was degassed and purged with H₂ 3 times, then the mixture was stirred under H₂ (50.0 Psi) at 50° C. for 2 hrs. The reaction mixture was filtered and the filtrate was concentrated under vacuum. The crude product was purified by silica gel chromatography (SiO₂, dichloromethane:methanol=15:1 to 10:1). rac-(R)-3-(6-(4-fluoro-4-(hydroxymethyl)piperidin-1-yl)pyridin-3-yl)piperidine-2,6-dione (1.17 g, 3.64 mmol, 70.0% yield) was obtained as an off-white solid. ¹H NMR: (400 MHz, MeOD) δ 7.97 (d, J=2.0 Hz, 1H), 7.46 (dd, J=2.4, 8.8 Hz, 1H), 6.88 (d, J=8.8 Hz, 1H), 4.09 (d, J=12.8 Hz, 2H), 3.79 (dd, J=11.6, 11.6 Hz, 1H), 3.56 (d, J=20 Hz, 2H), 3.27-3.21 (m, 2H), 2.77-2.63 (m, 2H), 2.27-2.10 (m, 2H), 1.91-1.85 (m, 2H), 1.805-1.63 (m, 2H).

### Step 4: Synthesis ofrac-(R)-1-(5-(2,6-dioxopiperidin-3-yl)pyridin-2-yl)-4-fluoropiperidine-4-carbaldehyde

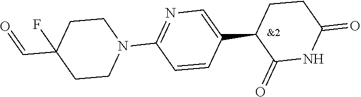 [SMILES: O=CC1(F)CCN(c2ccc([C@H]3CCC(=O)NC3=O)cn2)CC1]

### rac-(R)-3-(6-(4-fluoro-4-(hydroxymethyl)piperidin-1-yl)pyridin-3-yl)piperidine-2,6-dione

To a solution ofrac-(R)-3-(6-(4-fluoro-4-(hydroxymethyl)piperidin-1-yl)pyridin-3-yl)piperidine-2,6-dione (1.20 g, 3.72 mmol, 1.00 eq) in DCM (24.0 mL) cooled to 0° C. was added Dess-Martin periodinane (3.16 g, 7.45 mmol, 2.31 mL, 2.00 eq), and the mixture was stirred at 20° C. for 12 hrs. The mixture was quenched with saturated Na₂SO₃ solution (50.0 mL), extracted with DCM:EtOH=10:1 (4×50.0 mL), then the organic layer was dried over Na₂SO₄, filtered, and concentrated under vacuum. The crude product was purified by Prep-HPLC (column: Phenomenex luna C18 150*25 mm*10 um; mobile phase: [water(FA)-ACN]; B %: 4%-34%, 10 min), then concentrated under vacuum to remove ACN and concentrated by lyophilization to yield the product rac-(R)-1-(5-(2,6-dioxopiperidin-3-yl)pyridin-2-yl)-4-fluoropiperidine-4-carbaldehyde (265 mg, 822 umol, 26.2% yield, 99.0% purity) as a white solid. LCMS: C₁₆H₁₈FN₃O₃ requires: 319.1. found: m/z=338.1 [M+H₂O+H]+. 1H NMR: (400 MHz, CDCl₃) δ 9.77 (d, J=4.8 Hz, 1H), 8.08 (d, J=2.4 Hz, 1H), 8.03 (s, 1H), 7.38 (dd, J=2.4, 8.8 Hz, 1H), 6.74 (d, J=8.8 Hz, 1H), 4.25-4.21 (m, 2H), 3.70 (dd, J=5.6, 10.8 Hz, 1H) 3.38-3.32 (m, 2H), 2.79-2.73 (m, 2H), 2.27-2.23 (m, 2H), 1.95-1.88 (m, 4H).

### Step 5: Synthesis of the Title Compound

To a scintillation vial was added 2-[5-(4-{2,6-diazaspiro[3.4]octane-2-carbonyl}-4-phenylpiperidin-1-yl)pyridazin-3-yl]phenol (15.00 mg, 0.0381 mmol), triethylamine (26.06 μL, 0.1906 mmol), 1-{4-[(3R)-2,6-dioxopiperidin-3-yl]phenyl}piperidine-4-carbaldehyde (13.74 mg, 0.0457 mmol) and DCE (3.00 mL) Stir at r.t. and add sodium triacetoxyborohydride (28.28 mg, 0.1334 mmol). Stir until complete consumption of starting material by MS. This reaction was complete and clean after 90 minutes. Quench the reaction by adding bicarbonate solution and extract with EtOAc. Dry the organic layer with brine and then over Na₂SO₄. Concentrated and the solution was injected onto RP-FC and purified with a gradient of 0-70% MeCN in H₂O to furnish the title compound (7.7 mg, 44%). LCMS: C₄₃H₄₇N₈O₄F requires: 758.4. found: m/z=759.4 [M+H]⁺.

### Example 20

### rac-(3R)-3-{4-[4-(6-{1-[6-(2-hydroxyphenyl)pyridazin-4-yl]-4-phenylpiperidine-4-carbonyl}-2,6-diazaspiro[3.3]heptane-2-carbonyl)piperidin-1-yl]phenyl}piperidine-2,6-dione

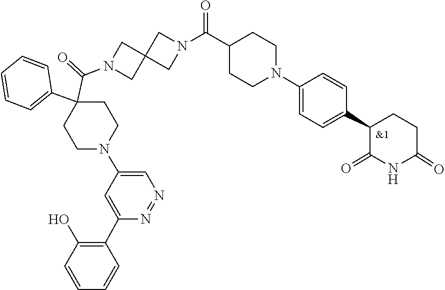 [SMILES: O=C1CC[C@H](c2ccc(N3CCC(C(=O)N4CC5(C4)CN(C(=O)C4(c6ccccc6)CCN(c6cnnc(-c7ccccc7O)c6)CC4)C5)CC3)cc2)C(=O)N1]

### Step 1: Synthesis of tert-butyl 1-(4-bromophenyl)piperidine-4-carboxylate

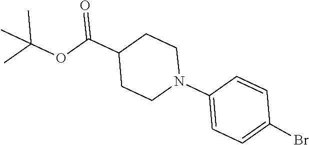 [SMILES: CC(C)(C)OC(=O)C1CCN(c2ccc(Br)cc2)CC1]

To a solution of 1-bromo-4-iodobenzene (50.0 g, 176 mmol, 1.00 eq) and tert-butyl piperidine-4-carboxylate (36.0 g, 194 mmol, 1.10 eq) in DMSO (500 mL) was added L-hydroxyproline (9.27 g, 70.7 mmol, 0.400 eq), K₂CO₃ (48.8 g, 353 mmol, 2.00 eq) and CuI (6.73 g, 35.3 mmol, 0.200 eq) under N₂. The reaction was stirred at 90° C. for 12 hrs. The reaction mixture was poured into H₂O (500 mL), then was extracted with ethyl acetate (3×500 mL). The combined organic layer was washed with brine (2×500 mL), dried over Na₂SO₄, filtered, and concentrated.

The residue was purified by column chromatography (SiO₂, Petroleum ether/ethyl acetate=100/1-50/1-40/1). tert-butyl 1-(4-bromophenyl)piperidine-4-carboxylate (43.0 g, 107 mmol, 50.6% yield, 84.9% purity) was obtained as a white solid. LCMS: C₁₆H₂₂BrNO₂ requires: 339.1. found: m/z=340.1 [M+H]⁺. ¹H NMR: (400 MHz, DMSO) δ 7.47-7.29 (m, 2H), 6.89-6.75 (m, 2H), 3.62-3.57 (m, 2H), 2.78-2.72 (m, 2H), 2.38-2.36 (m, 1H), 1.86-1.82 (m, 2H), 1.60-1.56 (m, 2H), 1.40 (s, 9H).

### Step 2: Synthesis of tert-butyl 1-(4-(2,6-bis(benzyloxy)pyridin-3-yl)phenyl)piperidine-4-carboxylate

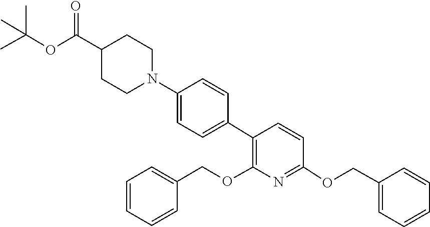 [SMILES: CC(C)(C)OC(=O)C1CCN(c2ccc(-c3ccc(OCc4ccccc4)nc3OCc3ccccc3)cc2)CC1]

To a solution of (2,6-bis(benzyloxy)pyridin-3-yl)boronic acid (47.5 g, 114 mmol, 1.20 eq) in dioxane (400 mL) and H₂O (80 mL) was added tert-butyl 1-(4-bromophenyl)piperidine-4-carboxylate (38.0 g, 94.8 mmol, 84.9% purity, 1.00 eq), K₂CO₃ (39.3 g, 284 mmol, 3.00 eq), and Pd(dppf)Cl₂ (3.47 g, 4.74 mmol, 0.05 eq) at 20° C. The reaction mixture was stirred at 90° C. for 12 hrs. The reaction mixture was poured into H₂O (500 mL), then was extracted with ethyl acetate (3×500 mL). The combined organic layer was washed with brine (2×500 mL), dried over Na₂SO₄, filtered, and concentrated. The residue was purified by column chromatography (SiO₂, petroleum ether/ethyl acetate=100/1-50/1-40/1). tert-butyl 1-(4-(2,6-bis(benzyloxy)pyridin-3-yl)phenyl)piperidine-4-carboxylate (46.0 g, 81.2 mmol, 75.8% yield, 97.2% purity) was obtained as a white solid. LCMS: C₃₅H₃₈N₂O₄ requires: 550.3. found: m/z=551.2 [M+H]⁺. ¹H NMR (400 MHz, DMSO) δ 7.60 (d, J=8.0 Hz, 2H), 7.50 (d, J=8.4 Hz, 1H), 7.43-7.35 (m, 10H), 6.97 (d, J=8.4 Hz, 2H), 6.47 (d, J=8.4 Hz, 1H), 5.45 (s, 2H), 5.37 (s, 2H), 3.71-3.66 (m, 2H), 2.86-2.79 (m, 2H), 2.40-2.35 (m, 1H), 2.03-1.99 (m, 2H), 1.90-1.85 (m, 2H), 1.48 (s, 9H).

### Step 3: Synthesis of rac-tert-butyl (R)-1-(4-(2,6-dioxopiperidin-3-yl)phenyl)piperidine-4-carboxylate

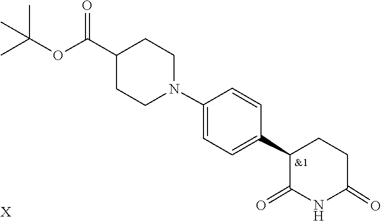

To a solution of tert-butyl 1-(4-(2,6-bis(benzyloxy)pyridin-3-yl)phenyl)piperidine-4-carboxylate (41.0 g, 72.4 mmol, 97.2% purity, 1.00 eq) in THF (410 mL) was added Pd/C (10.0 g, 72.4 mmol, 10.0% purity, 1.00 eq) under N₂. The suspension was degassed under vacuum and purged with H₂ 3 times. The reaction mixture was stirred under H₂ (50 psi) at 25° C. for 12 hrs. The suspension was filtered through a pad of celite and the pad was washed with THF (4×500 mL).

The solution was concentrated under reduced pressure using a rotary evaporator. rac-tert-butyl (R)-1-(4-(2,6-dioxopiperidin-3-yl)phenyl)piperidine-4-carboxylate (30.0 g, 77.6 mmol, 95.7% yield, 96.3% purity) was obtained as a white solid. LCMS: C₂₁H₂₈N₂O₄ requires: 372.2. found: m/z=373.2 [M+H]⁺. ¹H NMR (400 MHz, DMSO) δ 10.77 (s, 1H), 7.03 (d, J=8.4 Hz, 1H), 6.88 (d, J=8.8 Hz, 1H), 3.73-3.69 (m, 1H), 3.58 (d, J=12.4 Hz, 2H), 2.75-2.70 (m, 2H), 2.68-2.62 (m, 1H), 2.43-3.36 (m, 2H), 2.20-2.00 (m, 2H), 1.86-1.83 (m, 2H), 1.61-1.59 (m, 2H), 1.40 (s, 9H).

### Step 4: Synthesis of rac-(R)-1-(4-(2,6-dioxopiperidin-3-yl)phenyl)piperidine-4-carboxylic acid

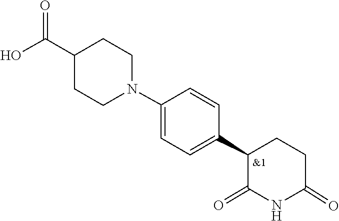 [SMILES: O=C1CC[C@H](c2ccc(N3CCC(C(=O)O)CC3)cc2)C(=O)N1]

To a solution of rac-tert-butyl (R)-1-(4-(2,6-dioxopiperidin-3-yl)phenyl)piperidine-4-carboxylate (10.0 g, 25.8 mmol, 96.3% purity, 1.00 eq) in DCM (270 mL) was added TFA (29.5 g, 258 mmol, 19.1 mL, 10.0 eq) at 25° C. Then the reaction mixture was stirred at 25° C. for 12 hrs.

The reaction mixture was concentrated under vacuum. The residue was treated with petroleum ether/ethyl acetate=1/1 (40.0 mL) at 25° C. for 10 mins, then filtered, and the filter cake was concentrated under vacuum. rac-(R)-1-(4-(2,6-dioxopiperidin-3-yl)phenyl)piperidine-4-carboxylic acid (9.00 g, 20.3 mmol, 60.5% yield, 97.3% purity, TFA salt) was obtained as a white solid. LCMS: C₁₇H₂₀N₂O₄ requires: 316.1. found: m/z=317.2 [M+H]⁺. ¹H NMR (400 MHz, D₂O) δ 7.61 (d, J=8.8 Hz, 2H), 7.50 (d, J=8.4 Hz, 2H), 4.08-4.03 (m, 1H), 3.78-3.74 (m, 2H), 3.71-3.67 (m, 2H), 2.85-2.80 (m, 1H), 2.77-2.75 (m, 2H), 2.37-2.15 (m, 6H).

### Step 5: Synthesis of the title compound

Stirred 2-[5-(4-{2,6-diazaspiro[3.3]heptane-2-carbonyl}-4-phenylpiperidin-1-yl)pyridazin-3-yl]phenol (10.00 mg, 0.0220 mmol), [(dimethylamino)({[1,2,3]triazolo[4,5-b]pyridin-3-yloxy})methylidene]dimethylazanium; hexafluoro-lambda5-phosphanuide (20.87 mg, 0.0549 mmol), and N,N-diisopropylethylamine (19.17 μL, 14.19 mg, 0.1098 mmol) in DMF. Add rac-1-{4-[(3R)-2,6-dioxopiperidin-3-yl]phenyl}piperidine-4-carboxylic acid and let stir at r.t. until complete by LCMS. the solution was injected onto RP-FC and purified with a gradient of 0-70% MeCN in H₂O to furnish the title compound (4.6 mg, 28%). LCMS: C₄₄H₄₇N₇O₄ requires: 753.4. found: m/z=754.4 [M+H]⁺.

### Example 21

### rac-(3R)-3-{4-[4-(1-{1-[6-(2-hydroxyphenyl)pyridazin-4-yl]-4-phenylpiperidine-4-carbonyl}-4-methylpiperidine-4-carbonyl)piperazin-1-yl]phenyl}piperidine-2,6-dione

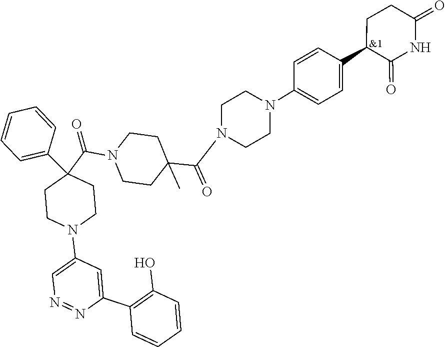 [SMILES: CC1(C(=O)N2CCN(c3ccc([C@H]4CCC(=O)NC4=O)cc3)CC2)CCN(C(=O)C2(c3ccccc3)CCN(c3cnnc(-c4ccccc4O)c3)CC2)CC1]

### Step 1: Synthesis of methyl 1-(1-(6-(2-hydroxyphenyl)pyridazin-4-yl)-4-phenylpiperidine-4-carbonyl)-4-methylpiperidine-4-carboxylate

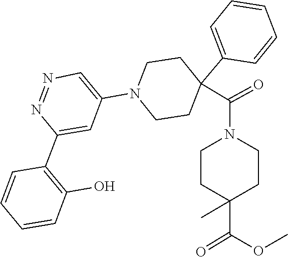 [SMILES: COC(=O)C1(C)CCN(C(=O)C2(c3ccccc3)CCN(c3cnnc(-c4ccccc4O)c3)CC2)CC1]

Stir 1-[6-(2-hydroxyphenyl)pyridazin-4-yl]-4-phenylpiperidine-4-carboxylic acid (71.00 mg, 0.1891 mmol), [(dimethylamino)({[1,2,3]triazolo[4,5-b]pyridin-3-yloxy})methylidene]dimethylazanium; hexafluoro-lambda5-phosphanuide (179.77 mg, 0.4728 mmol), and N,N-diisopropylethylamine (165.15 μL, 122.21 mg, 0.9456 mmol) in DMF. Add methyl 4-methylpiperidine-4-carboxylate (27.87 μL, 40.14 mg, 0.2553 mmol) and let stir at r.t. until complete by LCMS. Directly injected onto RP-FC and purified to furnish the title compound (80 mg, 73%) LCMS: C₃₀H₃₄N₄O₄ requires: 514.6 found: m/z=515.5 [M+H]⁺.

### Step 2: Synthesis of 1-{1-[6-(2-hydroxyphenyl)pyridazin-4-yl]-4-phenylpiperidine-4-carbonyl}-4-methylpiperidine-4-carboxylic acid

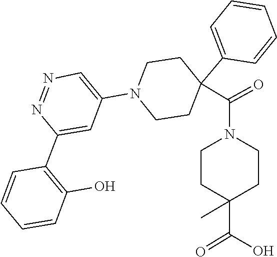 [SMILES: CC1(C(=O)O)CCN(C(=O)C2(c3ccccc3)CCN(c3cnnc(-c4ccccc4O)c3)CC2)CC1]

A mixture of methyl 1-{1-[6-(2-hydroxyphenyl)pyridazin-4-yl]-4-phenylpiperidine-4-carbonyl}-4-methylpiperidine-4-carboxylate (29.00 mg, 0.0564 mmol) and lithium hydroxide monohydrate (8.51 mg, 0.2029 mmol) in MeOH and H₂O was stirred for 2 h at r.t. The mixture was then acidified to pH=5 with HCl and extracted with EtoAC×3. The combined org washed with brine, dried over Na₂SO₄ and concentrated. Crude used in next step without further purification.

### Step 3: Synthesis of the Title Compound

Stirred 1-{1-[6-(2-hydroxyphenyl)pyridazin-4-yl]-4-phenylpiperidine-4-carbonyl}-4-methylpiperidine-4-carboxylic acid (10.10 mg, 0.0202 mmol), [(dimethylamino)({[1,2,3]triazolo[4,5-b]pyridin-3-yloxy})methylidene]dimethylazanium; hexafluoro-lambda5-phosphanuide (19.18 mg, 0.0504 mmol), and N,N-diisopropylethylamine (17.62 μL, 13.04 mg, 0.1009 mmol) in DMF. Add rac-(3R)-3-[4-(piperazin-1-yl)phenyl]piperidine-2,6-dione (6.62 mg, 0.0242 mmol) and let stir at r.t. until complete by LCMS. The solution was injected onto RP-FC and purified with a gradient of 0-70% MeCN in H₂O to furnish the title compound (5.9 mg, 35%). LCMS: C₄₄H₄₉N₇O₄ requires: 755.4. found: m/z=756.4 [M+H]⁺.

### Example 22

### rac-(3R)-3-(4-{4-[4-(4-{1-[6-(2-hydroxyphenyl)pyridazin-4-yl]-4-phenylpiperidine-4-carbonyl}piperazin-1-yl)butanoyl]piperazin-1-yl}phenyl)piperidine-2,6-dione

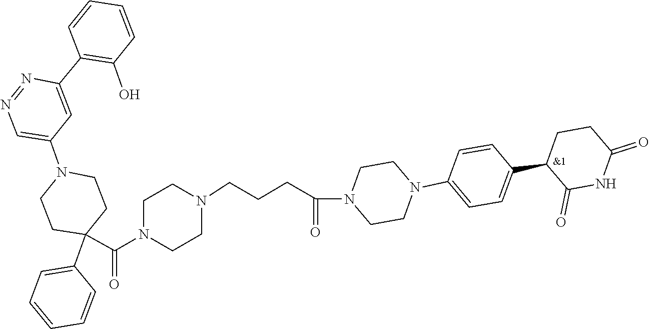 [SMILES: O=C1CC[C@H](c2ccc(N3CCN(C(=O)CCCN4CCN(C(=O)C5(c6ccccc6)CCN(c6cnnc(-c7ccccc7O)c6)CC5)CC4)CC3)cc2)C(=O)N1]

### Step 1: Synthesis of 4-(4-(1-(6-(2-hydroxyphenyl)pyridazin-4-yl)-4-phenylpiperidine-4-carbonyl)piperazin-1-yl)butanoic acid

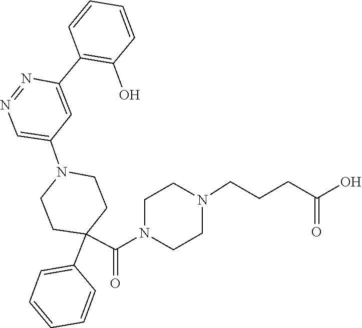 [SMILES: O=C(O)CCCN1CCN(C(=O)C2(c3ccccc3)CCN(c3cnnc(-c4ccccc4O)c3)CC2)CC1]

Stir 1-[6-(2-hydroxyphenyl)pyridazin-4-yl]-4-phenylpiperidine-4-carboxylic acid (17.00 mg, 0.0453 mmol), [chloro(dimethylamino)methylidene]dimethylazanium; hexafluoro-lambda5-phosphanuide (25.41 mg, 0.0906 mmol), and 1-methylimidazole (14.87 mg, 0.1811 mmol) in DMF. Add ethyl 4-(piperazin-1-yl)butanoate (9.98 mg, 0.0498 mmol) and let stir at r.t. until complete by LCMS. Directly inject onto RP-FC, purify to isolate the ester protected product, then dissolve with 1.2 equivalents of LiOH monohydrate in THF and concentrate to yield the final product. (25 mg, 90%) LCMS: C₃₀H₃₅N₅O₄ requires: 529.6. found: m/z=530.5 [M+H]⁺.

### Step 2: Synthesis of the Title Compound

Stirred rac-(3R)-3-[4-(piperazin-1-yl)phenyl]piperidine-2,6-dione (3.10 mg, 0.0113 mmol), [(dimethylamino)({[1,2,3]triazolo[4,5-b]pyridin-3-yloxy})methylidene]dimethylazanium; hexafluoro-lambda5-phosphanuide (8.97 mg, 0.0236 mmol), and N,N-diisopropylethylamine (8.24 μL, 6.10 mg, 0.0472 mmol) in DMF. Add 4-(4-{1-[6-(2-hydroxyphenyl)pyridazin-4-yl]-4-phenylpiperidine-4-carbonyl}piperazin-1-yl)butanoic acid (5.00 mg, 0.0094 mmol) and let stir at r.t. until complete by LCMS. The solution was injected onto RP-FC and purified with a gradient of 0-70% MeCN in H₂O to furnish the title compound (1.2 mg, 15%). LCMS: C₄₅H₅₂N₈O₅ requires: 784.4. found: m/z=785.4 [M+H]⁺.

### Example 23

### (3R)-3-(4-{4-[(6-{1-[6-(2-hydroxyphenyl)pyridazin-4-yl]-4-methylpiperidine-4-carbonyl}-2,6-diazaspiro[3.3]heptan-2-yl)methyl]piperidin-1-yl}phenyl)piperidine-2,6-dione

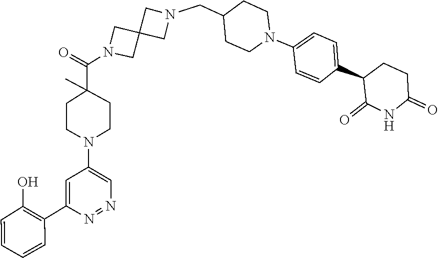 [SMILES: CC1(C(=O)N2CC3(CN(CC4CCN(c5ccc([C@H]6CCC(=O)NC6=O)cc5)CC4)C3)C2)CCN(c2cnnc(-c3ccccc3O)c2)CC1]

### Step 1: Synthesis of (1-(6-(2-hydroxyphenyl)pyridazin-4-yl)-4-methylpiperidin-4-yl)(2,6-diazaspiro[3.3]heptan-2-yl)methanone

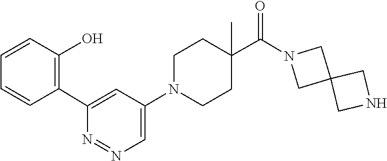 [SMILES: CC1(C(=O)N2CC3(CNC3)C2)CCN(c2cnnc(-c3ccccc3O)c2)CC1]

Stir 1-[6-(2-hydroxyphenyl)pyridazin-4-yl]-4-methylpiperidine-4-carboxylic acid (10.00 mg, 0.0319 mmol), [(dimethylamino)({[1,2,3]triazolo[4,5-b]pyridin-3-yloxy})methylidene]dimethylazanium; hexafluoro-lambda5-phosphanuide (24.27 mg, 0.0638 mmol), and N,N-diisopropylethylamine (22.30 μL, 16.50 mg, 0.1276 mmol) in DMF. Add tert-butyl 2,6-diazaspiro[3.3]heptane-2-carboxylate (6.33 mg, 0.0319 mmol) and let stir at r.t. until complete by LCMS. Directly purify Boc protected intermediate (6.1 mg, 38%) by RP-FC then dissolve in 1:1 TFA:DCM and stir for 30 m at r.t. Concentrate to yield product and carry to next step. LCMS: C₂₂H₂₇N₅O₂ requires: 393.3. found: m/z=394.3[M+H]⁺.

### Step 2: Synthesis of the Title Compound

To a scintillation vial was added 2-[5-(4-{2,6-diazaspiro[3.4]octane-2-carbonyl}-4-phenylpiperidin-1-yl)pyridazin-3-yl]phenol (15.00 mg, 0.0381 mmol), triethylamine (26.06 μL, 0.1906 mmol), 1-{4-[(3R)-2,6-dioxopiperidin-3-yl]phenyl}piperidine-4-carbaldehyde (13.74 mg, 0.0457 mmol) and DCE (3.00 mL) Stir at r.t. and add sodium triacetoxyborohydride (28.28 mg, 0.1334 mmol). Stir until complete consumption of starting material by MS. This reaction was complete and clean after 90 minutes. Quench the reaction by adding bicarbonate solution and extract with EtOAc. Dry the organic layer with brine and then over Na₂SO₄. Concentrated and the solution was injected onto RP-FC and purified with a gradient of 0-70% MeCN in H₂O to furnish the title compound (7.4 mg, 29%). LCMS: C₄₃H₄₇N₈O₄F requires: 677.4. found: m/z=678.4 [M+H]⁺.

### Example 24

### (3R)-3-(4-{4-[(6-{4-cyclopropyl-1-[6-(2-hydroxyphenyl)pyridazin-4-yl]piperidine-4-carbonyl}-2,6-diazaspiro[3.3]heptan-2-yl)methyl]piperidin-1-yl}phenyl)piperidine-2,6-dione

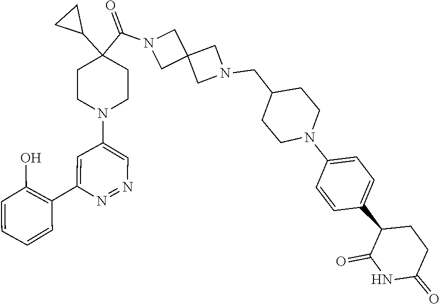 [SMILES: O=C1CC[C@H](c2ccc(N3CCC(CN4CC5(C4)CN(C(=O)C4(C6CC6)CCN(c6cnnc(-c7ccccc7O)c6)CC4)C5)CC3)cc2)C(=O)N1]

### Step 1: Synthesis of (4-cyclopropyl-1-(6-(2-hydroxyphenyl)pyridazin-4-yl)piperidin-4-yl)(2,6-diazaspiro[3.3]heptan-2-yl)methanone

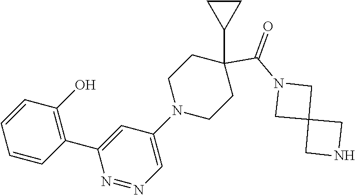 [SMILES: O=C(N1CC2(CNC2)C1)C1(C2CC2)CCN(c2cnnc(-c3ccccc3O)c2)CC1]

Stir 4-cyclopropyl-1-[6-(2-hydroxyphenyl)pyridazin-4-yl]piperidine-4-carboxylic acid (10.00 mg, 0.0295 mmol), [(dimethylamino)({[1,2,3]triazolo[4,5-b]pyridin-3-yloxy})methylidene]dimethylazanium; hexafluoro-lambda5-phosphanuide (22.41 mg, 0.0589 mmol), and N,N-diisopropylethylamine (20.58 μL, 15.23 mg, 0.1179 mmol) in DMF. Add tert-butyl 2,6-diazaspiro[3.3]heptane-2-carboxylate (5.84 mg, 0.0295 mmol) and let stir at r.t. until complete by LCMS. Directly inject and purify Boc intermediate via RP-FC (4.9 mg, 31%) then dissolve in 1:1 TFA:DCM and stir for 1 h at r.t., then concentrate and lyophilize to use in the next step. LCMS: C₂₄H₂₉N₅O₂ requires: 419.2. found: m/z=420.3 [M+H]⁺.

### Step 2: Synthesis of the Title Compound

To a scintillation vial was added 2-[5-(4-{2,6-diazaspiro[3.4]octane-2-carbonyl}-4-phenylpiperidin-1-yl)pyridazin-3-yl]phenol (15.00 mg, 0.0358 mmol), triethylamine (24.5 μL, 0.1788 mmol), 1-{4-[(3R)-2,6-dioxopiperidin-3-yl]phenyl}piperidine-4-carbaldehyde (13.0 mg, 0.0429 mmol) and DCE (3.00 mL) Stir at r.t. and add sodium triacetoxyborohydride (26.5 mg, 0.1251 mmol). Stir until complete consumption of starting material by MS. This reaction was complete and clean after 90 minutes. Quench the reaction by adding bicarbonate solution and extract with EtOAc. Dry the organic layer with brine and then over Na₂SO₄. Concentrated and the solution was injected onto RP-FC and purified with a gradient of 0-70% MeCN in H₂O to furnish the title compound (8.0 mg, 31%). LCMS: C₄₃H₄₇N₈O₄F requires: 704.4. found: m/z=705.4 [M+H]⁺.

### Example 25

### (3R)-3-(4-{4-[(8-fluoro-2-{1-[6-(2-hydroxyphenyl)pyridazin-4-yl]-4-phenylpiperidine-4-carbonyl}-2,6-diazaspiro[3.4]octan-6-yl)methyl]piperidin-1-yl}phenyl)piperidine-2,6-dione

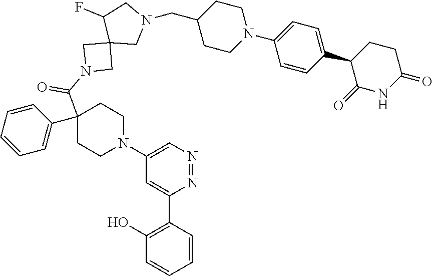 [SMILES: O=C1CC[C@H](c2ccc(N3CCC(CN4CC(F)C5(C4)CN(C(=O)C4(c6ccccc6)CCN(c6cnnc(-c7ccccc7O)c6)CC4)C5)CC3)cc2)C(=O)N1]

### Step 1: Synthesis of (8-fluoro-2,6-diazaspiro[3.4]octan-2-yl)(1-(6-(2-hydroxyphenyl)pyridazin-4-yl)-4-phenylpiperidin-4-yl)methanone

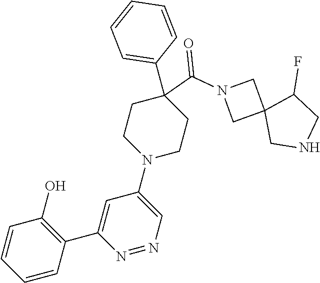

Stir 1-[6-(2-hydroxyphenyl)pyridazin-4-yl]-4-phenylpiperidine-4-carboxylic acid (40.00 mg, 0.1065 mmol), [(dimethylamino)({[1,2,3]triazolo[4,5-b]pyridin-3-yloxy})methylidene]dimethylazanium; hexafluoro-lambda5-phosphanuide (101.28 mg, 0.2664 mmol), and N,N-diisopropylethylamine (93.04 μL, 68.85 mg, 0.5327 mmol) in DMF. Add tert-butyl 8-fluoro-2,6-diazaspiro[3.4]octane-6-carboxylate (33.12 mg, 0.1438 mmol) and let stir at r.t. until complete by LCMS. Directly inject onto RP-FC and isolate the Boc intermediate (70 mg, 91%) and then dissolve in 1:1 TFA:DCM, stir for 1 h at r.t. and concentrate to yield the title product LCMS: C₂₇H₂₉N₅O₂F requires: 487.6. found: m/z=488.5 [M+H]⁺.

### Step 2: Synthesis of the Title Compound

To a scintillation vial was added 2-[5-(4-{8-fluoro-2,6-diazaspiro[3.4]octane-2-carbonyl}-4-phenylpiperidin-1-yl)pyridazin-3-yl]phenol (130 mg, 0.26 mmol), triethylamine (182 μL, 1.33 mmol), 1-{4-[(3R)-2,6-dioxopiperidin-3-yl]phenyl}piperidine-4-carbaldehyde (96 mg, 0.32 mmol) and DCE (3.00 mL) Stir at r.t. and add sodium triacetoxyborohydride (198 mg, 0.93 mmol). Stir until complete consumption of starting material by MS. This reaction was complete and clean after 90 minutes. Quench the reaction by adding bicarbonate solution and extract with EtOAc. Dry the organic layer with brine and then over Na₂SO₄. Concentrated and the solution was injected onto RP-FC and purified with a gradient of 0-70% MeCN in H₂O to furnish the title compound (84 mg, 40%). LCMS: C₄₅H₅₀N₇O₄F requires: 771.4. found: m/z=772.6 [M+H]⁺.

¹H NMR (500 MHz, DMSO) δ 10.70 (s, 1H), 9.57 (s, 1H), 8.89 (d, J=2.9 Hz, 1H), 7.97 (d, J=8.0 Hz, 1H), 7.48 (d, J=2.9 Hz, 1H), 7.35 (t, J=7.6 Hz, 1H), 7.31-7.24 (m, 3H), 7.00-6.94 (m, 3H), 6.89-6.79 (m, 4H), 5.69 (s, 1H), 4.09-3.90 (m, 2H), 3.68-3.62 (m, 2H), 3.57 (d, J=12.2 Hz, 1H), 3.50 (dt, J=12.6, 4.1 Hz, 2H), 3.39-3.21 (m, 2H), 2.74 (td, J=11.7, 2.9 Hz, 2H), 2.59-2.49 (m, 4H), 2.39 (dt, J=16.9, 4.5 Hz, 3H), 2.31 (s, 2H), 2.10-2.01 (m, 2H), 1.97-1.90 (m, 3H), 1.88-1.82 (m, 3H), 1.66 (dd, J=30.0, 8.2 Hz, 1H), 1.51 (dtd, J=14.4, 10.9, 3.9 Hz, 3H), 1.28-0.99 (m, 2H).

### Example 26

### rac-(3R)-3-(4-{4-[(6-{4-benzyl-1-[6-(2-hydroxyphenyl)pyridazin-4-yl]piperidine-4-carbonyl}-2,6-diazaspiro[3.3]heptan-2-yl)methyl]piperidin-1-yl}phenyl)piperidine-2,6-dione

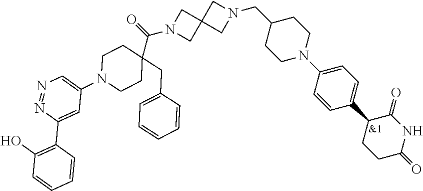 [SMILES: O=C1CC[C@@H](c2ccc(N3CCC(CN4CC5(C4)CN(C(=O)C4(Cc6ccccc6)CCN(c6cnnc(-c7ccccc7O)c6)CC4)C5)CC3)cc2)C(=O)N1]

### Step 1: Synthesis of 1-(tert-butyl) 4-ethyl 4-benzylpiperidine-1,4-dicarboxylate

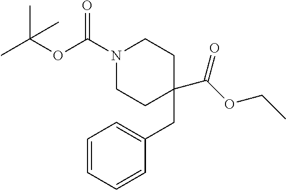 [SMILES: CCOC(=O)C1(Cc2ccccc2)CCN(C(=O)OC(C)(C)C)CC1]

To a stirring solution of 1-(tert-butyl) 4-ethyl piperidine-1,4-dicarboxylate (10.0 g, 38.9 mmol, 1.00 eq) in THF (100 mL) was cooled to −78° C. and LDA (2.00 M, 23.3 mL, 1.20 eq) was added. The reaction was allowed to stir for 0.5 hr. The reaction mixture was heated to 20° C. and benzyl bromide (6.65 g, 38.9 mmol, 4.62 mL, 1.00 eq) was added. The mixture was stirred at 20° C. for 2 hrs. The reaction was washed with saturated NH₄Cl solution (100 mL). The aqueous layers were extracted with EtOAc (2×120 mL). The organics were combined, washed with brine (250 mL) and dried over Na₂SO₄. The solution was filtered and evaporated under reduced pressure to a yellow oil. The crude product was used in the next step without further purification. LCMS: C₂₀H₂₉NO₄ requires: 347.2. found: m/z=248.2 [M−Boc+H]⁺.

### Step 2: Synthesis of ethyl 4-benzylpiperidine-4-carboxylate

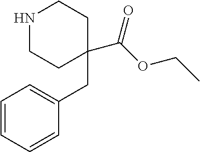 [SMILES: CCOC(=O)C1(Cc2ccccc2)CCNCC1]

To a solution of 1-(tert-butyl) 4-ethyl 4-benzylpiperidine-1,4-dicarboxylate (13.5 g, 38.9 mmol, 1.00 eq) in EtOAc (10.0 mL) was added HCl/EtOAc (4.00 M, 9.73 mL, 1.00 eq). The mixture was stirred at 20° C. for 2 hrs. The solution was filtered and evaporated under reduced pressure to a yellow oil. The crude product was used in the next step without further purification. LCMS: C₁₅H₂₁NO₂ requires: 247.2. found: m/z=248.4 [M+H]⁺.

### Step 3: Synthesis of ethyl 4-benzyl-1-(6-chloropyridazin-4-yl)piperidine-4-carboxylate

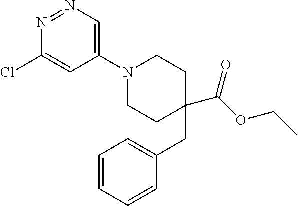 [SMILES: CCOC(=O)C1(Cc2ccccc2)CCN(c2cnnc(Cl)c2)CC1]

To a solution of ethyl 4-benzylpiperidine-4-carboxylate (5.52 g, 22.3 mmol, 1.00 eq) and 3,5-dichloropyridazine (6.48 g, 43.5 mmol, 1.95 eq) in IPA (90.0 mL) was added DIPEA (14.4 g, 111 mmol, 19.4 mL, 5.00 eq) at 20° C. The mixture was stirred at 70° C. for 16 hrs. The residue was diluted with H₂O (250 mL) and extracted with EtOAc (3×300 mL). The combined organic layers were washed with brine (3×500 mL), dried over Na₂SO₄, filtered, and concentrated under reduced pressure to give a residue. The residue was purified by column chromatography (SiO₂, Petroleum ether/Ethyl acetate=10/1 to 0/1, R_{f}=0.20) to furnish the title compound as a yellow solid (3.44 g, 9.17 mmol, 41.1% yield, 96.4% purity). LCMS: C₁₉H₂₂ClN₃O₂ requires: 359.1. found: m/z=360.2 [M+H]⁺.

### Step 4: Synthesis of ethyl 4-benzyl-1-(6-(2-hydroxyphenyl)pyridazin-4-yl)piperidine-4-carboxylate

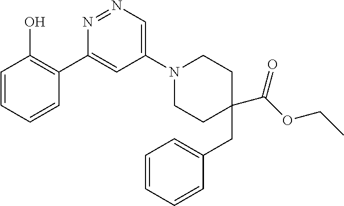 [SMILES: CCOC(=O)C1(Cc2ccccc2)CCN(c2cnnc(-c3ccccc3O)c2)CC1]

To a solution of ethyl 4-benzyl-1-(6-chloropyridazin-4-yl)piperidine-4-carboxylate (2.00 g, 5.56 mmol, 1.00 eq), (2-hydroxyphenyl)boronic acid (1.53 g, 11.1 mmol, 2.00 eq) and Pd(dppf)Cl₂ (610 mg, 834 μmol, 0.150 eq) in dioxane (20.0 mL) was added K₂CO₃ (2.30 g, 16.6 mmol, 3.00 eq) in H₂O (2.00 mL) at 20° C. The mixture was stirred at 110° C. for 16 hrs. The residue was diluted with H₂O (20.0 mL) and extracted with EtOAc (3×30.0 mL). The combined organic layers were washed with brine (50.0 mL), dried over Na₂SO₄, filtered, and concentrated under reduced pressure to give a residue. The residue was purified by column chromatography (SiO₂, Petroleum ether/EtOAc=6/1 to 3/1) to furnish the title compound as a yellow solid (1.20 g, 2.87 mmol, 51.7% yield). LCMS: C₂₅H₂₇N₃O₃ requires: 417.2. found: m/z=418.3 [M+H]⁺.

### Step 5: Synthesis of 4-benzyl-1-(6-(2-hydroxyphenyl)pyridazin-4-yl)piperidine-4-carboxylic acid

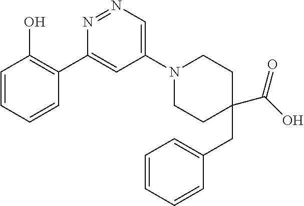 [SMILES: O=C(O)C1(Cc2ccccc2)CCN(c2cnnc(-c3ccccc3O)c2)CC1]

To a solution of ethyl 4-benzyl-1-(6-(2-hydroxyphenyl)pyridazin-4-yl)piperidine-4-carboxylate (500 mg, 1.20 mmol, 1.00 eq) in THF (2.50 mL) and MeOH (2.50 mL) was added LiOH·H₂O (603 mg, 14.4 mmol, 12.0 eq) in H₂O (2.50 mL) at 20° C. The mixture was stirred at 50° C. for 20 hrs. The reaction mixture was cooled down to room temperature and filter under reduced pressure with MTBE (30.0 mL). H₂O (15.0 mL) was added and pH was adjusted to 5 with 1 M HCl (7.00 mL), filtered, and concentrated under reduced pressure to give a residue. The title compound was obtained as a pink solid (603 mg, 1.52 mmol, 61.8% yield, 98.8% purity). LCMS: C₂₃H₂₃N₃O₃ requires: 389.2. found: m/z=390.3 [M+H]⁺. ¹H NMR (400 MHz, DMSO-d₆) δ 13.51-13.30 (m, 1H), 8.93 (d, J=2.0 Hz, 1H), 8.00 (br d, J=7.6 Hz, 1H), 7.53 (br d, J=2.4 Hz, 1H), 7.34 (br t, J=7.6 Hz, 1H), 7.27-7.24 (m, 3H), 7.13 (br d, J=7.2 Hz, 2H), 6.96-6.93 (m, 2H), 4.14 (br d, J=13.2 Hz, 2H), 3.12 (br t, J=12.0 Hz, 2H), 2.85 (s, 2H), 2.10-1.96 (m, 2H), 1.57 (br t, J=10.4 Hz, 2H).

### Step 6: Synthesis of the Title Compound

4-benzyl-1-[6-(2-hydroxyphenyl)pyridazin-4-yl]piperidine-4-carboxylic acid (20.00 mg, 0.0514 mmol), PyBOP; hexafluoro-lambda5-phosphanuide (34.74 mg, 0.0668 mmol), and N,N-diisopropylethylamine (44.85 μL, 33.19 mg, 0.2568 mmol) were stirred in dimethylformamide (0.50 mL, 0.47 g, 6.4300 mmol) at room temperature for 1 h. rac-(3R)-3-[4-(4-{2,6-diazaspiro[3.3]heptan-2-ylmethyl}piperidin-1-yl)phenyl]piperidine-2,6-dione (25.54 mg, 0.0668 mmol) was added and the reaction stirred overnight. The solution was injected onto RP-FC and purified with a gradient of 0-70% MeCN in H₂O to furnish the title compound (8.6 mg, 22%). LCMS: C₄₅H₅₁N₇O₄ requires: 753.4. found: m/z=754.4 [M+H]⁺.

### Example 27

### rac-(3R)-3-{4-[4-({6-[4-(3-chlorophenyl)-1-[6-(2-hydroxyphenyl)pyridazin-4-yl]piperidine-4-carbonyl]-2,6-diazaspiro[3.3]heptan-2-yl}methyl)piperidin-1-yl]phenyl}piperidine-2,6-dione

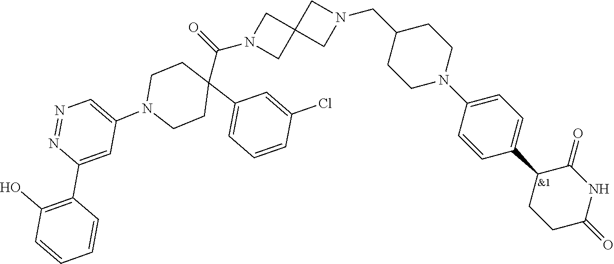 [SMILES: O=C1CC[C@@H](c2ccc(N3CCC(CN4CC5(C4)CN(C(=O)C4(c6cccc(Cl)c6)CCN(c6cnnc(-c7ccccc7O)c6)CC4)C5)CC3)cc2)C(=O)N1]

### Step 1: Synthesis of 1-(tert-butyl) 4-methyl 4-(3-chlorophenyl)piperidine-1,4-dicarboxylate

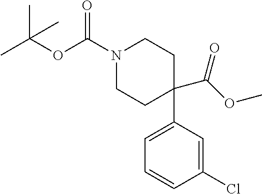 [SMILES: COC(=O)C1(c2cccc(Cl)c2)CCN(C(=O)OC(C)(C)C)CC1]

To a solution of methyl 2-(3-chlorophenyl)acetate (5.00 g, 27.1 mmol, 1.00 eq) in DMF (50.0 mL) was added NaH (2.38 g, 59.6 mmol, 60% purity, 2.20 eq) at 0° C. under N₂, the mixture was stirred at 0° C. for 0.5 hr. Then tert-butyl bis(2-chloroethyl)carbamate (7.87 g, 32.5 mmol, 1.20 eq) was added, the mixture was heated to 60° C. and stirred at 60° C. for 2 hrs. The mixture was cooled down to 25° C., then poured into saturated NH₄Cl (100 mL) aqueous solution, then extracted with DCM (3×50.0 mL). The combined organic layers were washed with brine (3×100 mL), dried over Na₂SO₄, filtered, and concentrated to give the product. The residue was purified by column chromatography (SiO₂, Petroleum ether/Ethyl acetate=I/O to 5/1, R_{f}=0.43) to furnish the title compound as a yellow solid (4.70 g, 12.4 mmol, 45.9% yield, 93.5% purity). LCMS: C₁₈H₂₄ClNO₄ requires: 353.1. found: m/z=298.1 [M−55]⁺. ¹H NMR (400 MHz, CDCl₃) δ 7.36 (s, 1H), 7.27 (br s, 3H), 3.99 (br d, J=12.0 Hz, 2H), 3.70 (s, 3H), 3.01 (br t, J=12.0 Hz, 2H), 2.51 (br d, J=12.8 Hz, 2H), 1.91-1.74 (m, 2H), 1.47 (s, 9H).

### Step 2: Synthesis of methyl 4-(3-chlorophenyl)piperidine-4-carboxylate

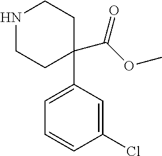 [SMILES: COC(=O)C1(c2cccc(Cl)c2)CCNCC1]

A solution of 1-(tert-butyl) 4-methyl 4-(3-chlorophenyl)piperidine-1,4-dicarboxylate (4.70 g, 12.4 mmol, 1.00 eq) in HCl/EtOAc (4 M, 23.5 mL, 7.57 eq) was stirred at 25° C. for 12 hrs. The mixture was concentrated. The title compound was obtained as a white solid (3.40 g, 11.7 mmol, 94.3% yield, HCl). LCMS: C₁₃H₁₆ClNO₂ requires: 253.1. found: m/z=254.4 [M+H]⁺. ¹H NMR (400 MHz, DMSO-d₆) δ 8.96 (br s, 2H), 7.47-7.31 (m, 4H), 3.65 (s, 3H), 3.22 (br d, J=12.8 Hz, 2H), 2.95 (br d, J=4.8 Hz, 2H), 2.55 (br d, J=14.8 Hz, 2H), 2.16 (br t, J=11.6 Hz, 2H).

### Step 3: Synthesis of methyl 4-(3-chlorophenyl)-1-(6-chloropyridazin-4-yl)piperidine-4-carboxylate

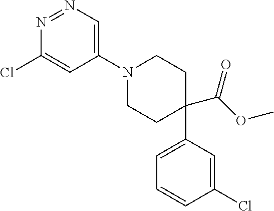 [SMILES: COC(=O)C1(c2cccc(Cl)c2)CCN(c2cnnc(Cl)c2)CC1]

To a solution of methyl 4-(3-chlorophenyl)piperidine-4-carboxylate (3.20 g, 11.0 mmol, 1.00 eq, HCl) in IPA (32.0 mL) was added DIEA (5.70 g, 44.1 mmol, 7.68 mL, 4.00 eq) at 25° C., then 3,5-dichloropyridazine (1.97 g, 13.2 mmol, 1.20 eq) was added, the mixture was heated to 70° C. and stirred at 70° C. for 2 hrs. The mixture was concentrated, then dissolved with DCM (30.0 mL). The mixture was washed with saturated aqueous NH₄Cl solution (4×50.0 mL) and brine (2×50.0 mL), dried over Na₂SO₄, filtered, and concentrated. The residue was purified by column chromatography (SiO₂, Petroleum ether/Ethyl acetate=1/0 to 0/1, Petroleum ether/Ethyl acetate=0/1, R_{f}=0.50), and to furnish the title compound as a yellow solid (2.40 g, 6.55 mmol, 59.4% yield). LCMS: C₁₇H₁₇C₁₂N₃O₂ requires: 365.1. found: m/z=366.1 [M+H]⁺. ¹H NMR (400 MHz, CDCl₃) δ 8.98 (d, J=2.4 Hz, 1H), 7.50-7.26 (m, 4H), 7.11 (d, J=2.4 Hz, 1H), 3.99 (br d, J=13.6 Hz, 2H), 3.66 (s, 3H), 3.14 (br t, J=11.2 Hz, 2H), 2.46 (br s, 2H), 2.05-1.84 (m, 2H).

### Step 4: Synthesis of methyl 4-(3-chlorophenyl)-1-(6-(2-hydroxyphenyl)pyridazin-4-yl)piperidine-4-carboxylate

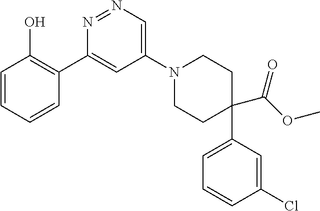 [SMILES: COC(=O)C1(c2cccc(Cl)c2)CCN(c2cnnc(-c3ccccc3O)c2)CC1]

To a solution of methyl 4-(3-chlorophenyl)-1-(6-chloropyridazin-4-yl)piperidine-4-carboxylate (2.20 g, 6.01 mmol, 1.00 eq) and (2-hydroxyphenyl)boronic acid (911 mg, 6.61 mmol, 1.10 eq) in dioxane (22.0 mL) and H₂O (2.20 mL) was added K₂CO₃ (2.49 g, 18.0 mmol, 3.00 eq) and BrettPhos Pd G₃ (272 mg, 300 μmol, 0.05 eq) at 25° C., then the mixture was heated to 80° C. and stirred for 12 hrs. The mixture was cooled down to 25° C., BrettPhos Pd G₃ (272 mg, 300 μmol, 0.05 eq) was added, then the mixture was heated to 80° C. and stirred at 80° C. for 12 hrs. The mixture was filtered, then the filtrate was poured into H₂O (100 mL) and extracted with EtOAc (3×50.0 mL). The organic layers were washed with brine (2×200 mL), dried over Na₂SO₄, filtered, and concentrated. The resulting residue was purified by column chromatography (SiO₂, Petroleum ether/Ethyl acetate=1/0 to 1/1, R_{f}=0.50) and concentrated to afford the title compound as a yellow solid (1.00 g, 2.36 mmol, 39.3% yield). LCMS: C₂₃H₂₂ClN₃O₃ requires: 423.1. found: m/z=424.1 [M+H]⁺. ¹H NMR (400 MHz, DMSO-d₆) δ 14.57 (s, 1H), 8.98 (d, J=2.8 Hz, 1H), 8.16-8.04 (m, 1H), 7.58 (d, J=2.4 Hz, 1H), 7.46-7.28 (m, 5H), 6.92 (d, J=8.4 Hz, 2H), 4.16 (br d, J=14.0 Hz, 2H), 3.68 (s, 3H), 3.22 (br t, J=11.6 Hz, 2H), 2.55 (br d, J=6.0 Hz, 2H), 2.09-2.00 (m, 2H).

### Step 5: Synthesis of 4-(3-chlorophenyl)-1-(6-(2-hydroxyphenyl)pyridazin-4-yl)piperidine-4-carboxylic acid

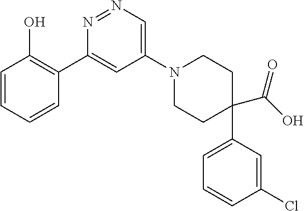 [SMILES: O=C(O)C1(c2cccc(Cl)c2)CCN(c2cnnc(-c3ccccc3O)c2)CC1]

To a solution of methyl 4-(3-chlorophenyl)-1-(6-(2-hydroxyphenyl)pyridazin-4-yl)piperidine-4-carboxylate (900 mg, 2.12 mmol, 1.00 eq) in MeOH (9.00 mL) was added NaOH (1 M, 6.37 mL, 3.00 eq) at 25° C., then the mixture was heated to 50° C. and stirred for 12 hrs. The pH of the mixture was adjusted to 4 with 4 M HCl aqueous solution, then filtered and concentrated. The residue was purified by prep-HPLC (column: Phenomenex luna C18 150*25 mm*10 um; mobile phase: [water (FA)-ACN]; gradient: 18%-48% B over 10 min), concentrated and lyophilized to afford the title compound as a yellow solid (402 mg, 938 μmol, 44.2% yield, 95.6% purity). LCMS: C₂₂H₂₀ClN₃O₃ requires: 409.1. found: m/z=410.1 [M+H]⁺. ¹H NMR (400 MHz, MeOD) δ 8.83 (d, J=2.8 Hz, 1H), 7.79 (br d, J=7.6 Hz, 1H), 7.52-7.46 (m, 2H), 7.45-7.26 (m, 4H), 7.04-6.94 (m, 2H), 4.20 (br d, J=14.0 Hz, 2H), 3.44 (br t, J=12.0 Hz, 2H), 2.71 (br d, J=13.2 Hz, 2H), 2.10-1.95 (m, 2H).

### Step 6: Synthesis of the Title Compound

4-(3-chlorophenyl)-1-[6-(2-hydroxyphenyl)pyridazin-4-yl]piperidine-4-carboxylic acid (20.00 mg, 0.0488 mmol), PyBOP; hexafluoro-lambda5-phosphanuide (33.01 mg, 0.0634 mmol), and N,N-diisopropylethylamine (42.61 μL, 31.53 mg, 0.2440 mmol) stirred in dimethylformamide (0.50 mL, 0.47 g, 6.4300 mmol) at rt for 1 h. Added rac-(3R)-3-[4-(4-{2,6-diazaspiro[3.3]heptan-2-ylmethyl}piperidin-1-yl)phenyl]piperidine-2,6-dione (24.26 mg, 0.0634 mmol) and stirred overnight. The solution was injected onto RP-FC and purified with a gradient of 0-70% MeCN in H₂O to furnish the title compound (4.0 mg, 10%). LCMS: C₄₄H₄₈N₇O₄C₁ requires: 773.3. found: m/z=774.3 [M+H]⁺.

### Example 28

### 1-[6-(2-hydroxyphenyl)pyridazin-4-yl]-4-phenyl-n-[(1r*,4r*)-4-(4-{4-[(3RS)-2,6-dioxopiperidin-3-yl]phenyl}piperazine-1-carbonyl)cyclohexyl]piperidine-4-carboxamide

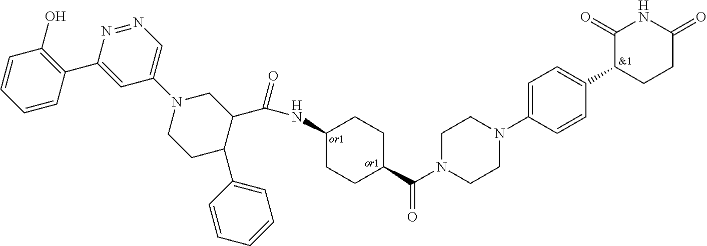 [SMILES: O=C1CC[C@H](c2ccc(N3CCN(C(=O)[C@H]4CC[C@@H](NC(=O)C5CN(c6cnnc(-c7ccccc7O)c6)CCC5c5ccccc5)CC4)CC3)cc2)C(=O)N1]

### Step 1: Synthesis of (3RS)-3-(4-{4-[(1r*,4r*)-4-aminocyclohexanecarbonyl]piperazin-1-yl}phenyl)piperidine-2,6-dione

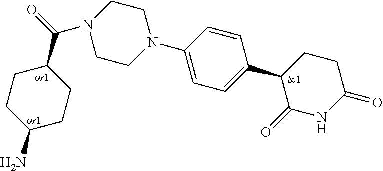 [SMILES: N[C@H]1CC[C@@H](C(=O)N2CCN(c3ccc([C@H]4CCC(=O)NC4=O)cc3)CC2)CC1]

rac-(3R)-3-[4-(piperazin-1-yl)phenyl]piperidine-2,6-dione (94.37 mg, 0.3452 mmol), [(dimethylamino)({[1,2,3]triazolo[4,5-b]pyridin-3-yloxy})methylidene]dimethylazanium; hexafluoro-lambda5-phosphanuide (273.49 mg, 0.7193 mmol), and N,N-diisopropylethylamine (251.25 μL, 185.93 mg, 1.4385 mmol) were stirred in DMF. Added (1s,4s)-4-[(tert-butoxycarbonyl)amino]cyclohexane-1-carboxylic acid (70.00 mg, 0.2877 mmol) and let stir at room temperature until complete by LCMS. Directly injected on RP-FC and purify with a gradient of 5-80% MeCN in H₂O to yield the Boc-protected intermediate (80 mg, 56%). This material was dissolved in 1:1 TFA:DCM and stirred for 1 h at room temperature, then concentrated and lyophilized to use in the next step. LCMS: C₂₂H₃₀N₄O₃ requires: 398.2. found: m/z=398.3 [M+H]⁺.

### Step 2: Synthesis of the Title Compound

1-[6-(2-hydroxyphenyl)pyridazin-4-yl]-4-phenylpiperidine-4-carboxylic acid (50.00 mg, 0.1332 mmol), PyBOP; hexafluoro-lambda5-phosphanuide (103.96 mg, 0.1998 mmol), and N,N-diisopropylethylamine (116.31 μL, 86.07 mg, 0.6659 mmol) were stirred in DMF for 1 hour at room temperature. (3RS)-3-(4-{4-[(1r*,4r*)-4-aminocyclohexanecarbonyl]piperazin-1-yl}phenyl)piperidine-2,6-dione (69.00 mg, 0.1731 mmol) was then added and the reaction was stirred overnight. The solution was injected onto RP-FC and purified with a gradient of 0-70% MeCN in H₂O to furnish the title compound (80.3 mg, 78%). LCMS: C₄₄H₄₉N₇O₅ requires: 755.4, found: m/z=756.8 [M+H]⁺.

### Example 29

### rac-(3R)-3-{4-[4-(5-{1-[6-(2-hydroxyphenyl)pyridazin-4-yl]-4-phenylpiperidine-4-carbonyl}-5-azaspiro[2.4]heptane-1-carbonyl)piperazin-1-yl]phenyl}piperidine-2,6-dione

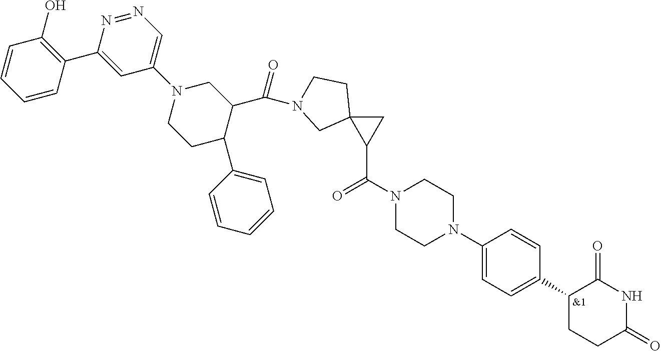 [SMILES: O=C1CC[C@H](c2ccc(N3CCN(C(=O)C4CC45CCN(C(=O)C4CN(c6cnnc(-c7ccccc7O)c6)CCC4c4ccccc4)C5)CC3)cc2)C(=O)N1]

### Step 1: Synthesis of rac-(3R)-3-[4-(4-{5-azaspiro[2.4]heptane-1-carbonyl}piperazin-1-yl)phenyl]piperidine-2,6-dione

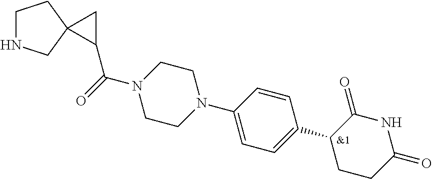 [SMILES: O=C1CC[C@H](c2ccc(N3CCN(C(=O)C4CC45CCNC5)CC3)cc2)C(=O)N1]

Stirred rac-(3R)-3-[4-(piperazin-1-yl)phenyl]piperidine-2,6-dione (95.16 mg, 0.3481 mmol), [(dimethylamino)({[1,2,3]triazolo[4,5-b]pyridin-3-yloxy})methylidene]dimethylazanium; hexafluoro-lambda5-phosphanuide (275.78 mg, 0.7253 mmol), and N,N-diisopropylethylamine (253.35 μL, 187.48 mg, 1.4506 mmol) in DMF. Added 5-(tert-butoxycarbonyl)-5-azaspiro[2.4]heptane-1-carboxylic acid (70.00 mg, 0.2901 mmol) and stirred at room temperature until complete by LCMS. Directly injected on RP-FC and purified with a gradient of 5-80% MeCN in H₂O to yield the Boc-protected intermediate (100 mg, 69%). Dissolved this material in 1:1 TFA:DCM and stirred for 1 h at room temperature, then concentrated and lyophilized to use in the next step. LCMS: C₂₂H₂₈N₄O₃ requires: 396.2. found: m/z=397.3 [M+H]⁺.

### Step 2: Synthesis of the Title Compound

1-[6-(2-hydroxyphenyl)pyridazin-4-yl]-4-phenylpiperidine-4-carboxylic acid (50.00 mg, 0.1332 mmol), PyBOP; hexafluoro-lambda5-phosphanuide (103.96 mg, 0.1998 mmol), and N,N-diisopropylethylamine (116.31 μL, 86.07 mg, 0.6659 mmol) were stirred in DMF for 1 hour at room temperature. rac-(3R)-3-[4-(4-{5-azaspiro[2.4]heptane-1-carbonyl}piperazin-1-yl)phenyl]piperidine-2,6-dione (68.65 mg, 0.1731 mmol) was then added and the reaction was stirred overnight. The solution was injected onto RP-FC and purified with a gradient of 0-70% MeCN in H₂O to furnish the title compound (29.3 mg, 25%) LCMS: C₄₄H₄₇N₇O₅ requires: 753.4. found: m/z=754.8 [M+H]⁺.

### Example 30

### rac-(3R)-3-{4-[4-(1-{1-[6-(2-hydroxyphenyl)pyridazin-4-yl]-4-phenylpiperidine-4-carbonyl}piperidine-4-carbonyl)piperazin-1-yl]phenyl}piperidine-2,6-dione

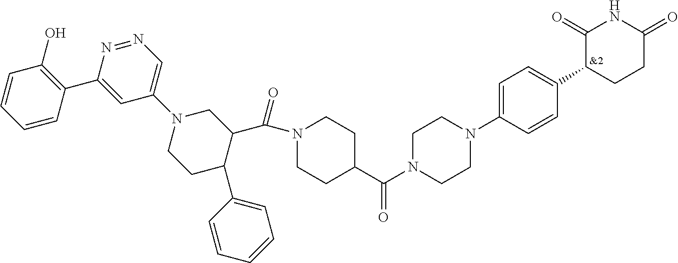 [SMILES: O=C1CC[C@H](c2ccc(N3CCN(C(=O)C4CCN(C(=O)C5CN(c6cnnc(-c7ccccc7O)c6)CCC5c5ccccc5)CC4)CC3)cc2)C(=O)N1]

### Step 1: Synthesis of rac-(3R)-3-{4-[4-(piperidine-4-carbonyl)piperazin-1-yl]phenyl}piperidine-2,6-dione

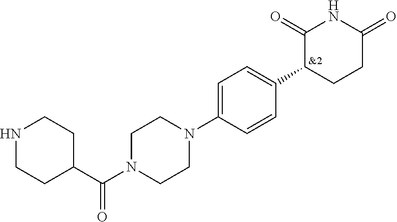 [SMILES: O=C1CC[C@H](c2ccc(N3CCN(C(=O)C4CCNCC4)CC3)cc2)C(=O)N1]

Stirred rac-(3R)-3-[4-(piperazin-1-yl)phenyl]piperidine-2,6-dione (100.14 mg, 0.3664 mmol), [(dimethylamino)({[1,2,3]triazolo[4,5-b]pyridin-3-yloxy})methylidene]dimethylazanium; hexafluoro-lambda5-phosphanuide (290.22 mg, 0.7633 mmol), and N,N-diisopropylethylamine (266.62 μL, 197.30 mg, 1.5265 mmol) in DMF. Added 1-(tert-butoxycarbonyl)piperidine-4-carboxylic acid (70.00 mg, 0.3053 mmol) and stirred at room temperature until complete by LCMS. Directly injected on RP-FC and purified with a gradient of 5-80% MeCN in H₂O to yield the Boc-protected intermediate (100 mg, 68%). Dissolved this material in 1:1 TFA:DCM and stirred for 1 h at room temperature, then concentrated and lyophilized to use in the next step. LCMS: C₂₁H₂₈N₄O₃ requires: 384.2. found: m/z=385.3 [M+H]⁺.

### Step 2: Synthesis of the Title Compound

1-[6-(2-hydroxyphenyl)pyridazin-4-yl]-4-phenylpiperidine-4-carboxylic acid (50.00 mg, 0.1332 mmol), PyBOP; hexafluoro-lambda5-phosphanuide (103.96 mg, 0.1998 mmol), and N,N-diisopropylethylamine (116.31 μL, 86.07 mg, 0.6659 mmol) were stirred in DMF for 1 hour at room temperature. rac-(3R)-3-{4-[4-(piperidine-4-carbonyl)piperazin-1-yl]phenyl}piperidine-2,6-dione (68.65 mg, 0.1731 mmol) was then added and the reaction was stirred overnight. The solution was injected onto RP-FC and purified with a gradient of 0-70% MeCN in H₂O to furnish the title compound (7.3 mg, 7%). LCMS: C₄₃H₄₇N₇05 requires: 741.4. found: m/z=742.3 [M+H]⁺.

### Example 31

### rac-(3R)-3-{4-[4-(6-{1-[6-(2-hydroxyphenyl)pyridazin-4-yl]-4-phenylpiperidine-4-carbonyl}-6-azaspiro[3.4]octane-2-carbonyl)piperazin-1-yl]phenyl}piperidine-2,6-dione

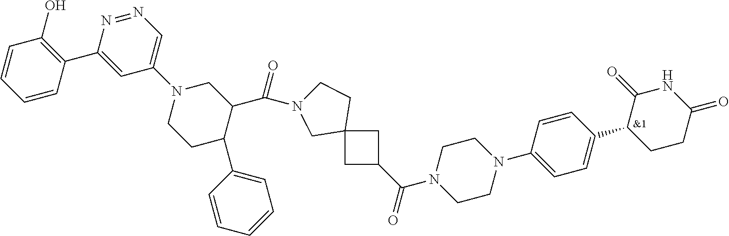 [SMILES: O=C1CC[C@H](c2ccc(N3CCN(C(=O)C4CC5(CCN(C(=O)C6CN(c7cnnc(-c8ccccc8O)c7)CCC6c6ccccc6)C5)C4)CC3)cc2)C(=O)N1]

### Step 1: Synthesis of rac-(3R)-3-[4-(4-{6-azaspiro[3.4]octane-2-carbonyl}piperazin-1-yl)phenyl]piperidine-2,6-dione

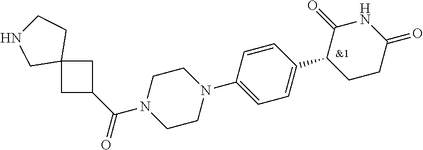 [SMILES: O=C1CC[C@H](c2ccc(N3CCN(C(=O)C4CC5(CCNC5)C4)CC3)cc2)C(=O)N1]

Stirred rac-(3R)-3-[4-(piperazin-1-yl)phenyl]piperidine-2,6-dione (89.93 mg, 0.3290 mmol), [(dimethylamino)({[1,2,3]triazolo[4,5-b]pyridin-3-yloxy})methylidene]dimethylazanium; hexafluoro-lambda5-phosphanuide (260.62 mg, 0.6854 mmol), and N,N-diisopropylethylamine (239.43 μL, 177.18 mg, 1.3709 mmol) in DMF. Added 6-(tert-butoxycarbonyl)-6-azaspiro[3.4]octane-2-carboxylic acid (70.00 mg, 0.2742 mmol) and stirred at room temperature until complete by LCMS. Directly injected on RP-FC and purified with a gradient of 5-80% MeCN in H₂O to yield the Boc-protected intermediate (100 mg, 68%). Dissolved this material in 1:1 TFA:DCM and stirred for 1 h at room temperature, then concentrated and lyophilized to use in the next step. LCMS: C₂₃H₃₀N₄O₃ requires: 410.2. found: m/z=411.3 [M+H]⁺.

### Step 2: Synthesis of the Title Compound

1-[6-(2-hydroxyphenyl)pyridazin-4-yl]-4-phenylpiperidine-4-carboxylic acid (50.00 mg, 0.1332 mmol), PyBOP; hexafluoro-lambda5-phosphanuide (103.96 mg, 0.1998 mmol), and N,N-diisopropylethylamine (116.31 μL, 86.07 mg, 0.6659 mmol) were stirred in DMF for 1 hour at room temperature. rac-(3R)-3-{4-[4-(piperidine-4-carbonyl)piperazin-1-yl]phenyl}piperidine-2,6-dione (68.65 mg, 0.1731 mmol) was then added and the reaction was stirred overnight. The solution was injected onto RP-FC and purified with a gradient of 0-70% MeCN in H₂O to furnish the title compound (51.7 mg, 46%) LCMS: C₄₅H₄₉N₇O₅ requires: 767.4. found: m/z=768.8 [M+H]⁺.

### Example 32

### rac-(3R)-3-{4-[4-({6-[4-(3,4-dichlorophenyl)-1-[6-(2-hydroxyphenyl)pyridazin-4-yl]piperidine-4-carbonyl]-2,6-diazaspiro[3.3]heptan-2-yl}methyl)piperidin-1-yl]phenyl}piperidine-2,6-dione

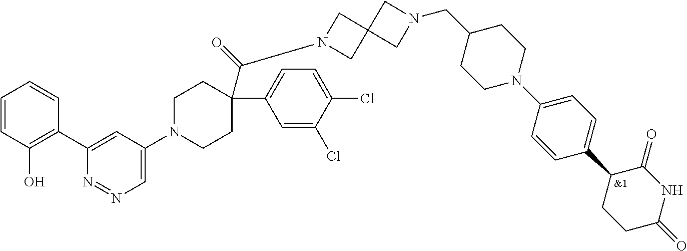 [SMILES: O=C1CC[C@@H](c2ccc(N3CCC(CN4CC5(C4)CN(C(=O)C4(c6ccc(Cl)c(Cl)c6)CCN(c6cnnc(-c7ccccc7O)c6)CC4)C5)CC3)cc2)C(=O)N1]

### Step 1: Synthesis of 1-(tert-butyl) 4-methyl 4-(3,4-dichlorophenyl)piperidine-1,4-dicarboxylate

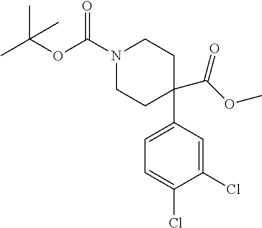 [SMILES: COC(=O)C1(c2ccc(Cl)c(Cl)c2)CCN(C(=O)OC(C)(C)C)CC1]

To a solution of methyl 2-(3,4-dichlorophenyl)acetate (10.0 g, 45.1 mmol, 1.00 eq) in DMF (100 mL) was added NaH (4.52 g, 112 mmol, 60.0% purity, 2.50 eq) at 0° C. The reaction mixture was stirred at 0° C. for 1 hr, then was added tert-butyl bis(2-chloroethyl)carbamate (12.0 g, 49.7 mmol, 1.10 eq) dropwise at 20° C. The reaction mixture was stirred at 60° C. for 3 hrs. The mixture was quenched with saturated aqueous NH₄Cl solution (200 mL), then extracted with EtOAc (2×100 mL). The organic layers were washed with brine (2×100 mL), dried over Na₂SO₄, filtered, and concentrated to give the title compound as a yellow oil (23.5 g, crude). LCMS: C₁₈H₂₃Cl₂NO₄ requires: 387.1. found: m/z=288.1 [M−100+H]⁺.

### Step 2: Synthesis of methyl 4-(3,4-dichlorophenyl)piperidine-4-carboxylate

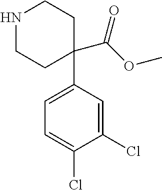 [SMILES: COC(=O)C1(c2ccc(Cl)c(Cl)c2)CCNCC1]

A solution of 1-(tert-butyl) 4-methyl 4-(3,4-dichlorophenyl)piperidine-1,4-dicarboxylate (23.0 g, 59.2 mmol, 1.00 eq) in HCl/EtOAc (4.00 M, 100 mL, 6.75 eq) was stirred at 25° C. for 12 hrs. The reaction mixture was concentrated under vacuum to give the title compound as a yellow oil (19.0 g, crude, HCl). LCMS: C₁₃H₁₅Cl₂NO₂ requires: 287.0. found: m/z=288.1 [M+H]⁺.

### Step 3: Synthesis of methyl 1-(6-chloropyridazin-4-yl)-4-(3,4-dichlorophenyl)piperidine-4-carboxylate

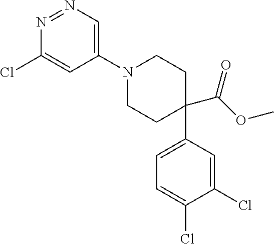 [SMILES: COC(=O)C1(c2ccc(Cl)c(Cl)c2)CCN(c2cnnc(Cl)c2)CC1]

To a solution of methyl 4-(3,4-dichlorophenyl)piperidine-4-carboxylate (17.0 g, 52.3 mmol, 1.00 eq, HCl) in IPA (170 mL) was added DIPEA (27.0 g, 209 mmol, 36.4 mL, 4.00 eq) at 20° C., then was added 3,5-dichloropyridazine (8.58 g, 57.6 mmol, 1.10 eq) at 20° C. The reaction mixture was concentrated under vacuum to give the residue, then was quenched with H₂O (200 mL), then extracted with DCM (3×100 mL). The organic layers were washed with brine (2×100 mL), dried over Na₂SO₄, filtered, and concentrated to give the product. The residue was purified by column chromatography (SiO₂, Petroleum ether/Ethyl acetate=I/O to 2/1) to afford the title compound as a yellow solid (2.20 g, 5.01 mmol, 9.56% yield, 91.2% purity). LCMS: C₁₇H₁₆C₁₃N₃O₂ requires: 399.0. found: m/z=400.1 [M+H]⁺. ¹H NMR (400 MHz, MeOD) δ 8.87 (d, J=2.8 Hz, 1H), 7.59-7.53 (m, 2H), 7.39 (dd, J=2.3, 8.6 Hz, 1H), 7.09 (d, J=2.8 Hz, 1H), 4.03-3.99 (m, 2H), 3.73 (s, 3H), 3.33-3.24 (m, 2H), 2.67 (br d, J=13.2 Hz, 2H), 2.07-1.99 (m, 2H).

### Step 4: Synthesis of methyl 4-(3,4-dichlorophenyl)-1-(6-(2-hydroxyphenyl)pyridazin-4-yl)piperidine-4-carboxylate

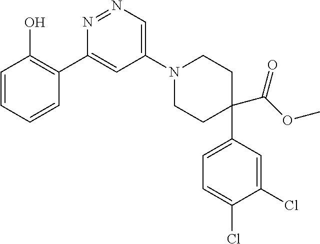 [SMILES: COC(=O)C1(c2ccc(Cl)c(Cl)c2)CCN(c2cnnc(-c3ccccc3O)c2)CC1]

To a solution of methyl 1-(6-chloropyridazin-4-yl)-4-(3,4-dichlorophenyl)piperidine-4-carboxylate (2.20 g, 5.01 mmol, 1.00 eq) in dioxane (22.0 mL) and H₂O (2.20 mL) was added K₂CO₃ (3.46 g, 25.0 mmol, 5.00 eq), (2-hydroxyphenyl)boronic acid (1.38 g, 10.0 mmol, 2.00 eq) and Pd(PPh₃)₂Cl₂ (527 mg, 751 μmol, 0.150 eq) under N₂ atmosphere at 20° C. The reaction mixture was stirred at 110° C. for 12 hrs. The reaction mixture was concentrated under vacuum, then was diluted with water (100 mL), extracted with DCM 30.0 mL (2×15.0 mL). The combined organic layers were washed with brine (2×50.0 mL), dried over Na₂SO₄, filtered, and concentrated. The residue was purified by column chromatography (SiO₂, Petroleum ether/Ethyl acetate=I/O to 1/1) to yield the title compound as a yellow solid (786 mg, 1.54 mmol, 28.2% yield, 89.8% purity). LCMS: C₂₃H₂₁Cl₂N₃O₃ requires: 457.1. found: m/z=458.2 [M+H]⁺. ¹H NMR (400 MHz, CDCl₃) δ 8.74 (d, J=2.8 Hz, 1H), 7.67 (dd, J=1.2, 8.0 Hz, 1H), 7.49-7.45 (m, 2H), 7.37-7.33 (m, 1H), 7.25 (dd, J=2.4, 8.4 Hz, 1H), 7.16 (d, J=2.8 Hz, 1H), 7.09 (d, J=7.6 Hz, 1H), 6.95-6.91 (m, 1H), 3.95 (br d, J=13.6 Hz, 2H), 3.76 (s, 3H), 3.30-3.23 (m, 2H), 2.73 (br d, J=13.6 Hz, 2H), 2.04-2.00 (m, 2H).

### Step 5: Synthesis of 4-(3,4-dichlorophenyl)-1-(6-(2-hydroxyphenyl)pyridazin-4-yl)piperidine-4-carboxylic acid

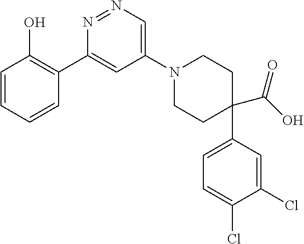 [SMILES: O=C(O)C1(c2ccc(Cl)c(Cl)c2)CCN(c2cnnc(-c3ccccc3O)c2)CC1]

To a solution of methyl 4-(3,4-dichlorophenyl)-1-(6-(2-hydroxyphenyl)pyridazin-4-yl)piperidine-4-carboxylate (786 mg, 1.54 mmol, 1.00 eq) in MeOH (4.00 mL), THF (4.00 mL) and H₂O (2.00 mL) was added NaOH (615 mg, 15.4 mmol, 10.0 eq) at 20-25° C. The reaction mixture was stirred at 80° C. for 12 hrs. The reaction mixture was evaporated under reduced pressure to remove the solvent, then the mixture was adjusted to pH=3 with 4 N HCl. Then filtered and collected the precipitate. The crude product was purified by prep-HPLC (column: Phenomenex luna C18 150*40 mm*15 um; mobile phase: [water (HCl)-ACN]; gradient: 20%-50% B over 10 min) to afford the title compound as a yellow solid (374 mg, 779 μmol, 50.6% yield, 100% purity, HCl). LCMS: C₂₂H₁₉Cl₂N₃O₃ requires: 443.1. found: m/z=444.2 [M+H]⁺. ¹H NMR (400 MHz, MeOD) δ 8.85 (br s, 1H), 7.62 (br s, 2H), 7.54-7.41 (m, 4H), 7.07-7.02 (m, 2H), 4.31 (br d, J=14.0 Hz, 2H), 3.53 (br t, J=12.4 Hz, 2H), 2.73 (br d, J=12.8 Hz, 2H), 2.07 (br t, J=12.8 Hz, 2H).

### Step 6: Synthesis of the Title Compound

4-(3,4-dichlorophenyl)-1-[6-(2-hydroxyphenyl)pyridazin-4-yl]piperidine-4-carboxylic acid (20.00 mg, 0.0450 mmol), PyBOP; hexafluoro-lambda5-phosphanuide (35.14 mg, 0.0675 mmol), and N,N-diisopropylethylamine (39.31 μL, 29.09 mg, 0.2251 mmol) were stirred in DMF for 1 hour at room temp. rac-(3R)-3-[4-(4-{2,6-diazaspiro[3.3]heptan-2-ylmethyl}piperidin-1-yl)phenyl]piperidine-2,6-dione (22.38 mg, 0.0585 mmol) was then added and the reaction was stirred overnight. The solution was injected onto RP-FC and purified with a gradient of 0-70% MeCN in H₂O to furnish the title compound (9.3 mg, 25%). LCMS: C₄₄H₄₇Cl₂N₇O₄ requires: 807.3, found: m/z=808.2 [M+H]⁺.

### Example 33

### rac-(3R)-3-(4-{4-[(6-{1-[6-(2-hydroxyphenyl)pyridazin-4-yl]-4-(2-methylphenyl)piperidine-4-carbonyl}-2,6-diazaspiro[3.3]heptan-2-yl)methyl]piperidin-1-yl}phenyl)piperidine-2,6-dione

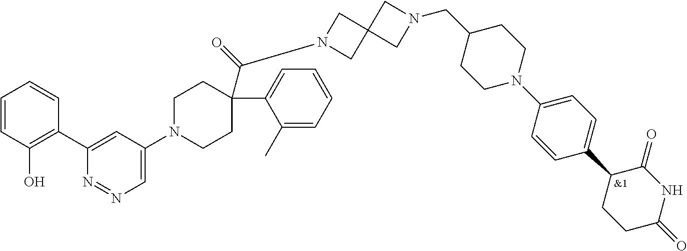 [SMILES: Cc1ccccc1C1(C(=O)N2CC3(CN(CC4CCN(c5ccc([C@@H]6CCC(=O)NC6=O)cc5)CC4)C3)C2)CCN(c2cnnc(-c3ccccc3O)c2)CC1]

### Step 1: Synthesis of 1-(tert-butyl) 4-methyl 4-(o-tolyl)piperidine-1,4-dicarboxylate

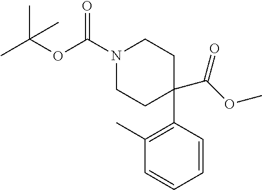 [SMILES: COC(=O)C1(c2ccccc2C)CCN(C(=O)OC(C)(C)C)CC1]

To a solution of 1-(tert-butyl) 4-methyl 4-(o-tolyl)piperidine-1,4-dicarboxylate (5.00 g, 30.4 mmol, 1.00 eq) in DMF (50.0 mL) was added NaH (3.04 g, 76.1 mmol, 60% purity, 2.50 eq) at 0° C. for 0.5 hr, then tert-butyl bis(2-chloroethyl)carbamate (8.85 g, 36.5 mmol, 1.20 eq) was added, the mixture was heated to 60° C. and stirred at 60° C. for 4 hrs. The reaction mixture was quenched with saturated NH₄Cl solution (20.0 mL) at 0° C. The mixture was poured into H₂O (500 mL) and extracted with EtOAc (3×200 mL). The combined organic phase was washed with brine (300 mL), dried over Na₂SO₄, filtered, and concentrated under vacuum. The title compound was obtained as a yellow oil (8.00 g, crude). LCMS: C₁₉H₂₇NO₄ requires: 333.2. found: m/z=334.2 [M−Boc+H]⁺.

### Step 2: Synthesis of methyl 4-(o-tolyl)piperidine-4-carboxylate

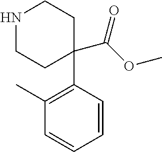

A solution of 1-(tert-butyl) 4-methyl 4-(o-tolyl)piperidine-1,4-dicarboxylate (8.00 g, 23.9 mmol, 1.00 eq) in HCl/dioxane (4 M, 59.9 mL, 10.0 eq) was stirred at 60° C. for 2 hrs. The mixture was concentrated under vacuum to give a residue. The title compound (10.3 g, 8.79 mmol, 36.6% yield, 19.9% purity) was obtained as black brown oil. LCMS: C₁₄H₁₉NO₂ requires: 233.1, found: m/z=234.2 [M+H]⁺.

### Step 3: Synthesis of methyl 1-(6-chloropyridazin-4-yl)-4-(o-tolyl)piperidine-4-carboxylate

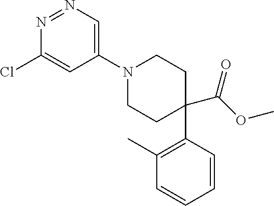 [SMILES: COC(=O)C1(c2ccccc2C)CCN(c2cnnc(Cl)c2)CC1]

To a solution of methyl 4-(o-tolyl)piperidine-4-carboxylate (7.30 g, 6.23 mmol, 1.00 eq) in IPA (70.0 mL) was added DIEA (3.22 g, 24.9 mmol, 4.34 mL, 4.00 eq) at 25° C., then 3,5-dichloropyridazine (1.11 g, 7.47 mmol, 1.20 eq) was added, the mixture was heated to 70° C. and stirred at 70° C. for 2 hrs. The mixture was poured into H₂O (100 mL) and extracted with ethyl acetate (3×50.0 mL). The combined organic phase was washed with brine (100 mL), dried over Na₂SO₄, filtered, and concentrated under vacuum. The residue was purified by column chromatography (SiO₂, Petroleum ether/Ethyl acetate=30/1 to 0/1, Petroleum ether/Ethyl acetate=1/1, R_{f}=0.20) to afford the title compound as a yellow oil (1.20 g, 2.93 mmol, 46.9% yield, 84.3% purity). LCMS: C₁₈H₂₀ClN₃O₂ requires: 345.1. found: m/z=346.2 [M+H]⁺.

### Step 4: Synthesis of methyl 1-(6-(2-hydroxyphenyl)pyridazin-4-yl)-4-(o-tolyl)piperidine-4-carboxylate

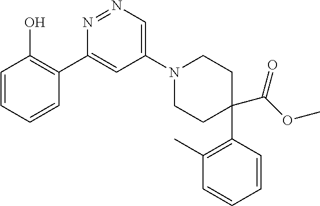 [SMILES: COC(=O)C1(c2ccccc2C)CCN(c2cnnc(-c3ccccc3O)c2)CC1]

To a solution of methyl 1-(6-chloropyridazin-4-yl)-4-(o-tolyl)piperidine-4-carboxylate (1.20 g, 2.93 mmol, 1.00 eq), (2-hydroxyphenyl)boronic acid (806 mg, 5.85 mmol, 2.00 eq), K₂CO₃ (1.62 g, 11.7 mmol, 4.00 eq) in dioxane (12.0 mL) and H₂O (1.20 mL) was added Pd(PPh₃)₂C₁₂ (307 mg, 438 μmol, 0.15 eq), the mixture was stirred at 110° C. for 2 hrs. The mixture was poured into H₂O (50 mL) and extracted with ethyl acetate (3×30.0 mL). The combined organic phase was washed with brine (50.0 mL), dried over Na₂SO₄, filtered, and concentrated under vacuum. The residue was purified by column chromatography (SiO₂, Petroleum ether/Ethyl acetate=10/1 to 1/1, Petroleum ether/Ethyl acetate=1/1, R_{f}=0.34) to yield the title compound as a yellow solid (1.00 g, 1.44 mmol, 49.3% yield, 58.2% purity). LCMS: C₂₄H₂₅N₃O₃ requires: 403.2. found: m/z=404.3 [M+H]⁺.

### Step 5: Synthesis of 1-(6-(2-hydroxyphenyl)pyridazin-4-yl)-4-(o-tolyl)piperidine-4-carboxylic acid

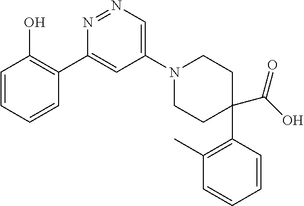 [SMILES: Cc1ccccc1C1(C(=O)O)CCN(c2cnnc(-c3ccccc3O)c2)CC1]

To a solution of methyl 1-(6-(2-hydroxyphenyl)pyridazin-4-yl)-4-(o-tolyl)piperidine-4-carboxylate (1.00 g, 1.44 mmol, 1.00 eq) in THF (5.00 mL), MeOH (5.00 mL), and H₂O (2.50 mL) was added NaOH (404 mg, 10.1 mmol, 7.00 eq). The mixture was stirred at 80° C. for 2 hrs. The mixture was concentrated under vacuum. The residue was purified by prep-HPLC (TFA condition: Phenomenex luna C18 150*40 mm*15 um; mobile phase: [water (TFA)-ACN]; gradient: 15%-45% B over 10 min) to yield the title compound as a yellow solid (360 mg, 924 μmol, 43.2% yield, 99.8% purity, TFA). LCMS: C₂₃H₂₃N₃O₃ requires: 389.2. found: m/z=390.2 [M+H]⁺. ¹H NMR: (400 MHz, DMSO-d₆) δ 8.82 (s, 1H), 7.2 (d, J=7.4 Hz, 1H), 7.50-7.41 (m, 3H), 7.19 (br s, 3H), 7.09-7.03 (m, 2H), 4.18-4.15 (m, 2H), 3.84 (s, 2H), 2.72 (br d, J=13.6 Hz, 2H), 2.39 (s, 3H), 2.31-2.23 (m, 2H).

### Step 6: Synthesis of the Title Compound

1-[6-(2-hydroxyphenyl)pyridazin-4-yl]-4-(2-methylphenyl)piperidine-4-carboxylic acid (20.00 mg, 0.0450 mmol), PyBOP; hexafluoro-lambda5-phosphanuide (35.14 mg, 0.0675 mmol), and N,N-diisopropylethylamine (39.31 μL, 29.09 mg, 0.2251 mmol) were stirred in DMF for 1 hour at room temperature. rac-(3R)-3-[4-(4-{2,6-diazaspiro[3.3]heptan-2-ylmethyl}piperidin-1-yl)phenyl]piperidine-2,6-dione (22.38 mg, 0.0585 mmol) was then added and the reaction was stirred overnight. The solution was injected onto RP-FC and purified with a gradient of 0-70% MeCN in H₂O to furnish the title compound (15.5 mg, 44%). LCMS: C₄₅H₅₁N₇O₄ requires: 753.4, found: m/z=754.5 [M+H]⁺.

### Example 34

### rac-(3R)-3-(4-{4-[(6-{1-[6-(2-hydroxyphenyl)pyridazin-4-yl]-4-(pyridin-3-yl)piperidine-4-carbonyl}-2,6-diazaspiro[3.3]heptan-2-yl)methyl]piperidin-1-yl}phenyl)piperidine-2,6-dione

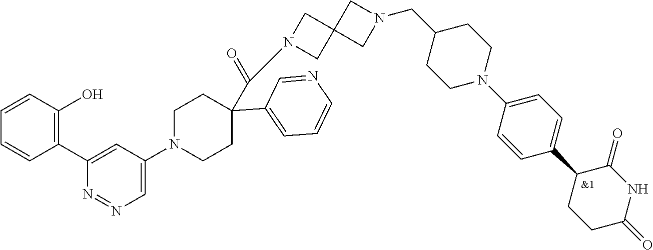 [SMILES: O=C1CC[C@@H](c2ccc(N3CCC(CN4CC5(C4)CN(C(=O)C4(c6cccnc6)CCN(c6cnnc(-c7ccccc7O)c6)CC4)C5)CC3)cc2)C(=O)N1]

### Step 1: Synthesis of tert-butyl 4-cyano-4-(pyridin-3-yl)piperidine-1-carboxylate

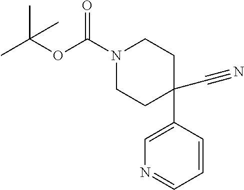 [SMILES: CC(C)(C)OC(=O)N1CCC(C#N)(c2cccnc2)CC1]

A flask was charged with sodium NaH (677 mg, 16.9 mmol, 60% purity, 1.00 eq) and DMF (40.0 mL) at 0° C. under N₂. tert-butyl bis(2-chloroethyl)carbamate (5.12 g, 21.1 mmol, 1.25 eq) was added in DMF (15.0 mL). Then tert-butyl 4-cyano-4-(pyridin-3-yl)piperidine-1-carboxylate (2.00 g, 16.9 mmol, 1.82 mL, 1.00 eq) was added in DMF (10.0 mL) dropwise. The reaction was allowed to stir at 0° C. for 2 hrs then warmed to 60° C. for 12 hrs. The reaction was quenched with 10% sodium bicarbonate (100 mL) and extracted with ethyl acetate (5×100 mL). The organic fractions were collected, washed with brine (100 mL), dried over sodium sulfate, and concentrated in vacuo. The residue was purified by silica gel chromatography (Petroleum ether/Ethyl acetate=5/1 to 1/1, TLC (Petroleum ether/Ethyl acetate=3:1) (I₂) R_{f}=0.57) to yield the title compound as a yellow oil (2.12 g, 7.38 mmol, 43.5% yield).

### Step 2: Synthesis of 4-(pyridin-3-yl)piperidine-4-carbonitrile

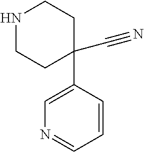 [SMILES: N#CC1(c2cccnc2)CCNCC1]

To a solution of tert-butyl 4-cyano-4-(pyridin-3-yl)piperidine-1-carboxylate (2.12 g, 7.38 mmol, 1 eq) in EtOAc (5.00 mL) was added HCl/EtOAc (4.00 M, 9.22 mL, 5.00 eq) at 15° C. The mixture was stirred at 15° C. for 1 hr. The mixture was filtered and the filter cake was washed with Petroleum ether (20.0 mL) and concentrated under vacuum. The title compound was obtained as a yellow solid (1.65 g, 7.38 mmol, crude, N/A purity, HCl). ¹H NMR: (400 MHz, DMSO-d₆) δ 9.79 (br d, J=7.6 Hz, 1H), 9.62 (br s, 1H), 8.99 (d, J=2.0 Hz, 1H), 8.90 (d, J=5.4 Hz, 1H), 8.54 (br d, J=8.0 Hz, 1H), 8.01 (dd, J=5.4, 8.0 Hz, 1H), 3.52 (br d, J=13.6 Hz, 2H), 3.09 (br d, J=7.6 Hz, 2H), 2.59-2.54 (m, 4H).

### Step 3: Synthesis of 1-(6-chloropyridazin-4-yl)-4-(pyridin-3-yl)piperidine-4-carbonitrile

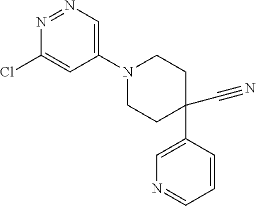 [SMILES: N#CC1(c2cccnc2)CCN(c2cnnc(Cl)c2)CC1]

To a solution of 4-(pyridin-3-yl)piperidine-4-carbonitrile (1.65 g, 7.38 mmol, 1.00 eq, HCl) and 3,5-dichloropyridazine (2.14 g, 14.3 mmol, 1.95 eq) in IPA (25 mL) was added DIEA (4.77 g, 36.8 mmol, 6.42 mL, 5.00 eq). The mixture was stirred at 70° C. for 16 hrs. The residue was diluted with H₂O (20.0 mL) and extracted with ethyl acetate 20.0 mL (3×20.0 mL). The combined organic layers were washed with brine (20.0 mL), dried over Na₂SO₄, filtered, and concentrated under reduced pressure to give a residue. The residue was purified by silica gel chromatography (eluted from Petroleum ether/Ethyl acetate=5/1 to 1/1, R_{f}=0.26) to yield the title compound as a gray oil (1.45 g, 4.67 mmol, 63.3% yield, 96.6% purity). LCMS: C₁₅H₁₄ClN₅ requires: 299.1. found: m/z=300.3 [M+H]⁺. ¹H NMR: (400 MHz, DMSO-d₆) δ 9.05 (d, J=2.8 Hz, 1H), 8.81 (d, J=2.4 Hz, 1H), 8.59 (dd, J=1.6, 4.8 Hz, 1H), 8.04-7.96 (m, 1H), 7.48 (dd, J=4.8, 8.0 Hz, 1H), 7.21 (d, J=2.8 Hz, 1H), 4.34 (br d, J=14.4 Hz, 2H), 3.29-3.20 (m, 2H), 2.36-2.28 (m, 2H), 2.23-2.13 (m, 2H).

### Step 4: Synthesis of 1-(6-(2-hydroxyphenyl)pyridazin-4-yl)-4-(pyridin-3-yl)piperidine-4-carbonitrile

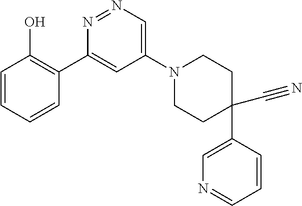 [SMILES: N#CC1(c2cccnc2)CCN(c2cnnc(-c3ccccc3O)c2)CC1]

To a solution of 1-(6-chloropyridazin-4-yl)-4-(pyridin-3-yl)piperidine-4-carbonitrile (1.45 g, 4.84 mmol, 1.00 eq) and (2-hydroxyphenyl)boronic acid (1.33 g, 9.67 mmol, 2 eq) in dioxane (26.0 mL) was added K₂CO₃ (2.01 g, 14.5 mmol, 3.00 eq) and Pd(dppf)Cl₂ (353 mg, 483 μmol, 0.10 eq) in H₂O (10.0 mL) at 20° C. The mixture was stirred at 120° C. for 16 hrs under N₂ atmosphere. The residue was diluted with H₂O (10.0 mL) and extracted with DCM (3×10.0 mL). The combined organic layers were washed with brine (10.0 mL), dried over Na₂SO₄, filtered, and concentrated under reduced pressure to give a residue. The residue was purified by silica gel chromatography (eluted from Petroleum ether/Ethyl acetate=5/1 to 1/1, R_{f}=0.27) to afford the title compound as a gray solid (1.40 g, 2.51 mmol, 51.9% yield, 64.1% purity). LCMS: C₂₁H₁₉N₅₀ requires: 357.2, found: m/z=358.3 [M+H]⁺. ¹H NMR: (400 MHz, DMSO-d₆) δ 14.5 (s, 1H), 9.05 (d, J=2.8 Hz, 1H), 8.83 (d, J=2.0 Hz, 1H), 8.60 (dd, J=1.6, 4.8 Hz, 1H), 8.15 (dd, J=1.2, 8.4 Hz, 1H), 8.04-8.00 (m, 1H), 7.68 (d, J=2.8 Hz, 1H), 7.51-7.48 (m, 1H), 7.37-7.31 (m, 1H), 6.95-6.92 (m, 2H), 4.54 (br d, J=14.0 Hz, 2H), 3.32-3.27 (m, 2H), 2.38-2.33 (m, 2H), 2.25-2.17 (m, 2H).

### Step 5: Synthesis of 1-(6-(2-hydroxyphenyl)pyridazin-4-yl)-4-(pyridin-3-yl)piperidine-4-carboxylic acid

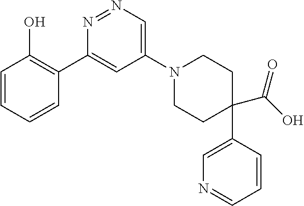 [SMILES: O=C(O)C1(c2cccnc2)CCN(c2cnnc(-c3ccccc3O)c2)CC1]

To a solution of 1-(6-(2-hydroxyphenyl)pyridazin-4-yl)-4-(pyridin-3-yl)piperidine-4-carbonitrile (1.40 g, 3.92 mmol, 1.00 eq) in H₂O (5.00 mL) and dioxane (20.0 mL) was added NaOH (700 mg, 17.5 mmol, 4.47 eq) at 20° C. The mixture was stirred at 70° C. for 16 hrs. The reaction mixture was filtered, the filter cake was soaked in water, and the filtrate was concentrated under vacuum. The crude product was purified by reversed-phase chromatography (water (NH₄HCO₃)-ACN condition). Separation gradient: 0%-30% B over 20 min. The title compound was obtained as a yellow solid (575.67 mg, 1.51 mmol, 38.58% yield, 98.8% purity). LCMS: C₂₁H₂₀N₄O₃ requires: 376.2. found: m/z=377.3 [M+H]⁺. ¹H NMR: (400 MHz, MeOD) δ 8.91-8.36 (m, 3H), 7.97 (br d, J=7.6 Hz, 1H), 7.79 (d, J=7.6 Hz, 1H), 7.43 (br d, J=1.6 Hz, 2H), 7.34 (t, J=7.4 Hz, 1H), 6.99-6.90 (m, 2H), 4.15 (br d, J=13.2 Hz, 2H), 3.42 (br t, J=12.4 Hz, 2H), 2.74 (br d, J=12.8 Hz, 2H), 2.00 (br t, J=10.4 Hz, 2H).

### Step 6: Synthesis of the Title Compound

1-[6-(2-hydroxyphenyl)pyridazin-4-yl]-4-(pyridin-3-yl)piperidine-4-carboxylic acid (20.00 mg, 0.0450 mmol), PyBOP; hexafluoro-lambda5-phosphanuide (35.14 mg, 0.0675 mmol), and N,N-diisopropylethylamine (39.31 μL, 29.09 mg, 0.2251 mmol) were stirred in DMF for 1 hour at room temperature. rac-(3R)-3-[4-(4-{2,6-diazaspiro[3.3]heptan-2-ylmethyl}piperidin-1-yl)phenyl]piperidine-2,6-dione (22.38 mg, 0.0585 mmol) was then added and the reaction was stirred overnight. The solution was injected onto RP-FC and purified with a gradient of 0-70% MeCN in H₂O to furnish the title compound (7.7 mg, 21%). LCMS: C₄₃H₄₈N₈O₄ requires: 740.4, found: m/z=741.1 [M+H]⁺.

### Example 35

### (3S)-3-[4-({1-[(1-{1-[6-(2-hydroxyphenyl)pyridazin-4-yl]-4-phenylpiperidine-4-carbonyl}piperidin-4-yl)methyl]piperidin-4-yl}methoxy)phenyl]piperidine-2,6-dione

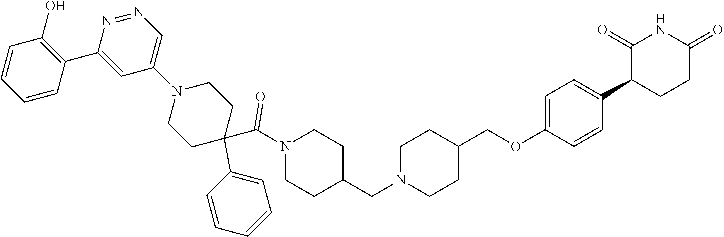 [SMILES: O=C1CC[C@@H](c2ccc(OCC3CCN(CC4CCN(C(=O)C5(c6ccccc6)CCN(c6cnnc(-c7ccccc7O)c6)CC5)CC4)CC3)cc2)C(=O)N1]

1-[6-(2-hydroxyphenyl)pyridazin-4-yl]-4-phenylpiperidine-4-carboxylic acid (30.00 mg, 0.0799 mmol), PyBOP; hexafluoro-lambda5-phosphanuide (62.38 mg, 0.1199 mmol), and N,N-diisopropylethylamine (69.78 μL, 51.64 mg, 0.3995 mmol) were stirred in DMF for 1 hour at room temperature. (3S)-3-(4-{[1-(piperidin-4-ylmethyl)piperidin-4-yl]methoxy}phenyl)piperidine-2,6-dione (38.31 mg, 0.0959 mmol) was then added and the reaction was stirred overnight. The solution was injected onto RP-FC and purified with a gradient of 0-70% MeCN in H₂O to furnish the title compound (4.9 mg, 7.81%). LCMS: C₄₅H₅₂N₆O₅ requires: 756.4. found: m/z=757.4 [M+H]⁺.

### Example 36

### (3R)-3-[4-({1-[(1-{1-[6-(2-hydroxyphenyl)pyridazin-4-yl]-4-phenylpiperidine-4-carbonyl}piperidin-4-yl)methyl]piperidin-4-yl}methoxy)phenyl]piperidine-2,6-dione

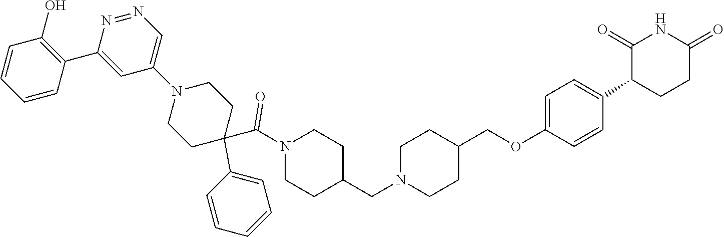 [SMILES: O=C1CC[C@H](c2ccc(OCC3CCN(CC4CCN(C(=O)C5(c6ccccc6)CCN(c6cnnc(-c7ccccc7O)c6)CC5)CC4)CC3)cc2)C(=O)N1]

1-[6-(2-hydroxyphenyl)pyridazin-4-yl]-4-phenylpiperidine-4-carboxylic acid (30.00 mg, 0.0799 mmol), PyBOP; hexafluoro-lambda5-phosphanuide (62.38 mg, 0.1199 mmol), and N,N-diisopropylethylamine (51.64 mg, 0.3995 mmol) were stirred in DMF for 1 hour at room temperature. (3R)-3-(4-{[1-(piperidin-4-ylmethyl)piperidin-4-yl]methoxy}phenyl)piperidine-2,6-dione (38.31 mg, 0.0959 mmol) was then added and the reaction was stirred overnight. The solution was injected onto RP-FC and purified with a gradient of 0-70% MeCN in H₂O to furnish the title compound (5.5 mg, 8.37%). LCMS: C₄₅H₅₂N₆O₅ requires: 756.4. found: m/z=757.4 [M+H]⁺.

### Example 37

### (3R)-3-[4-(4-{[2-(1-{1-[6-(2-hydroxyphenyl)pyridazin-4-yl]-4-phenylpiperidine-4-carbonyl}piperidin-3-yl)pyrrolidin-1-yl]methyl}piperidin-1-yl)phenyl]piperidine-2,6-dione

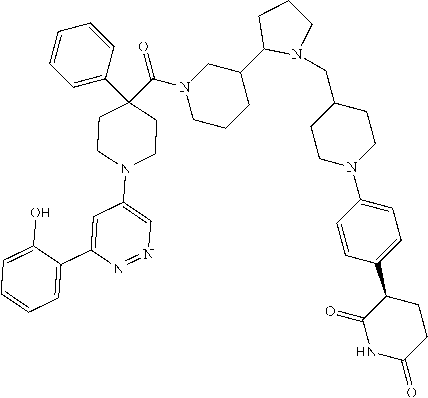 [SMILES: O=C1CC[C@H](c2ccc(N3CCC(CN4CCCC4C4CCCN(C(=O)C5(c6ccccc6)CCN(c6cnnc(-c7ccccc7O)c6)CC5)C4)CC3)cc2)C(=O)N1]

### Step 1: Synthesis of 2-(5-{4-phenyl-4-[3-(pyrrolidin-2-yl)piperidine-1-carbonyl]piperidin-1-yl}pyridazin-3-yl)phenol

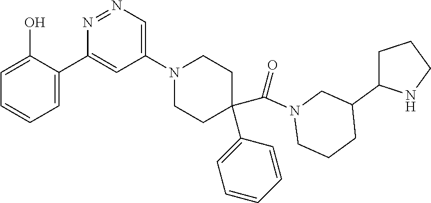 [SMILES: O=C(N1CCCC(C2CCCN2)C1)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (50.00 mg, 0.1332 mmol), [(dimethylamino)({[1,2,3]triazolo[4,5-b]pyridin-3-yloxy})methylidene]dimethylazanium; hexafluoro-lambda5-phosphanuide (126.60 mg, 0.3330 mmol), and N,N-diisopropylethylamine (116.31 μL, 86.07 mg, 0.6659 mmol) in DMF. Added tert-butyl 2-(piperidin-3-yl)pyrrolidine-1-carboxylate (45.74 mg, 0.1798 mmol) and stirred at room temperature until complete by LCMS. Directly injected on RP-FC and purified with a gradient of 5-80% MeCN in H₂O to yield the Boc-protected intermediate (25.8 mg, 32%). Dissolved this material in 1:1 TFA:DCM and stirred for 1 h at room temperature, then concentrated and lyophilized to use in the next step. LCMS: C₃₆H₄₅N₅O₄ requires: 611.3. found: m/z=612.4 [M+H]⁺.

### Step 2: Synthesis of the Title Compound

Dissolved 2-(5-{4-phenyl-4-[3-(pyrrolidin-2-yl)piperidine-1-carbonyl]piperidin-1-yl}pyridazin-3-yl)phenol (28.00 mg, 0.0547 mmol), 1-{4-[(3R)-2,6-dioxopiperidin-3-yl]phenyl}piperidine-4-carbaldehyde (18.08 mg, 0.0602 mmol), and N,N-diisopropylethylamine (47.79 μL, 35.36 mg, 0.2736 mmol) in DCE and stirred for 30 min at room temperature. Then added sodium triacetoxyborohydride (34.79 mg, 0.1642 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (19.4 mg, 45%). LCMS: C₄₈H₅₇N₇O₄ requires: 795.4. found: m/z=796.5 [M+H]⁺.

### Example 38

### (3R)-3-(4-{4-[(3-{1-[6-(2-hydroxyphenyl)pyridazin-4-yl]-4-phenylpiperidine-4-carbonyl}-3,8-diazabicyclo[4.2.0]octan-8-yl)methyl]piperidin-1-yl}phenyl)piperidine-2,6-dione

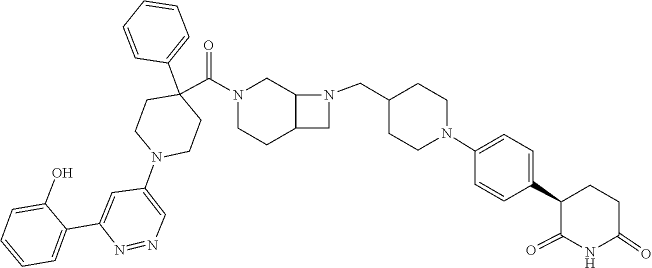 [SMILES: O=C1CC[C@H](c2ccc(N3CCC(CN4CC5CCN(C(=O)C6(c7ccccc7)CCN(c7cnnc(-c8ccccc8O)c7)CC6)CC54)CC3)cc2)C(=O)N1]

### Step 1: Synthesis of 2-[5-(4-{3,8-diazabicyclo[4.2.0]octane-3-carbonyl}-4-phenylpiperidin-1-yl)pyridazin-3-yl]phenol

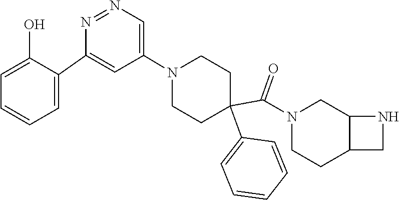 [SMILES: O=C(N1CCC2CNC2C1)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (50.00 mg, 0.1332 mmol), [(dimethylamino)({[1,2,3]triazolo[4,5-b]pyridin-3-yloxy})methylidene]dimethylazanium; hexafluoro-lambda5-phosphanuide (126.60 mg, 0.3330 mmol), and N,N-diisopropylethylamine (116.31 μL, 86.07 mg, 0.6659 mmol) in DMF. Added 8-(tert-butoxycarbonyl)-3,8-diazabicyclo[4.2.0]octan-3-ium (42.61 mg, 0.1998 mmol) and stirred at room temperature until complete by LCMS. Directly injected on RP-FC and purified with a gradient of 5-80% MeCN in H₂O to yield the Boc-protected intermediate (27.1 mg, 36%). Dissolved this material in 1:1 TFA:DCM and stirred for 1 h at room temperature, then concentrated and lyophilized to use in the next step. LCMS: C₂₈H₃₁N₅O₂ requires: 469.2. found: m/z=470.3 [M+H]⁺.

### Step 2: Synthesis of the Title Compound

Dissolved 2-[5-(4-{3,8-diazabicyclo[4.2.0]octane-3-carbonyl}-4-phenylpiperidin-1-yl)pyridazin-3-yl]phenol (28.60 mg, 0.0609 mmol), 1-{4-[(3R)-2,6-dioxopiperidin-3-yl]phenyl}piperidine-4-carbaldehyde (20.12 mg, 0.0670 mmol), and N,N-diisopropylethylamine (53.19 μL, 39.36 mg, 0.3045 mmol) in DCE and stirred for 30 min at room temperature. Then added sodium triacetoxyborohydride (38.72 mg, 0.1827 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (18.5 mg, 37%). LCMS: C₄₅H₅₁N₇O₄ requires: 753.4. found: m/z=754.4 [M+H]⁺.

### Example 39

### N-{5-[(1-{4-[(3R)-2,6-dioxopiperidin-3-yl]phenyl}piperidin-4-yl)methyl]-5-azaspiro[3.5]nonan-8-yl}-1-[6-(2-hydroxyphenyl)pyridazin-4-yl]-4-phenylpiperidine-4-carboxamide

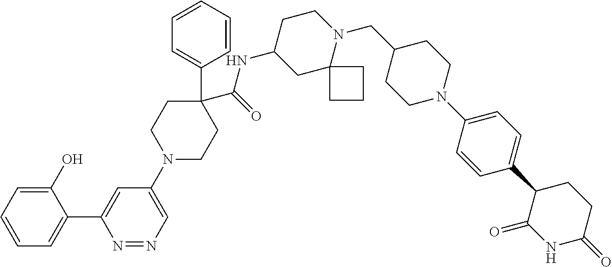 [SMILES: O=C1CC[C@H](c2ccc(N3CCC(CN4CCC(NC(=O)C5(c6ccccc6)CCN(c6cnnc(-c7ccccc7O)c6)CC5)CC45CCC5)CC3)cc2)C(=O)N1]

### Step 1: Synthesis of N-{5-azaspiro[3.5]nonan-8-yl}-1-[6-(2-hydroxyphenyl)pyridazin-4-yl]-4-phenylpiperidine-4-carboxamide

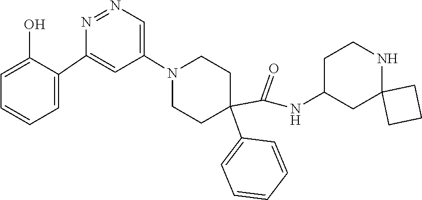 [SMILES: O=C(NC1CCNC2(CCC2)C1)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added tert-butyl 8-amino-5-azaspiro[3.5]nonane-5-carboxylate (42.35 mg, 0.1762 mmol) and stirred at room temperature until complete by LCMS. Directly injected on RP-FC and purified with a gradient of 5-80% MeCN in H₂O to yield the Boc-protected intermediate (35 mg, 44%). Dissolved this material in 1:1 TFA:DCM and stirred for 1 h at room temperature, then concentrated and lyophilized to use in the next step. LCMS: C₃₀H₃₅N₅O₂ requires: 497.3. found: m/z=498.3 [M+H]⁺.

### Step 2: Synthesis of the Title Compound

Dissolved N-{5-azaspiro[3.5]nonan-8-yl}-1-[6-(2-hydroxyphenyl)pyridazin-4-yl]-4-phenylpiperidine-4-carboxamide (35.70 mg, 0.0717 mmol), 1-{4-[(3R)-2,6-dioxopiperidin-3-yl]phenyl}piperidine-4-carbaldehyde (23.70 mg, 0.0789 mmol), and N,N-diisopropylethylamine (62.65 μL, 46.36 mg, 0.3587 mmol) in DCE and stirred for 30 min at room temperature. Then added sodium triacetoxyborohydride (45.61 mg, 0.2152 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (25.1 mg, 44%).

LCMS: C₄₇H₅₅N₇O₄ requires: 781.4. found: m/z=782.4 [M+H]⁺.

### Example 40

### (3R)-3-[4-(4-{[2-(1-{1-[6-(2-hydroxyphenyl)pyridazin-4-yl]-4-phenylpiperidine-4-carbonyl}piperidin-4-yl)pyrrolidin-1-yl]methyl}piperidin-1-yl)phenyl]piperidine-2,6-dione

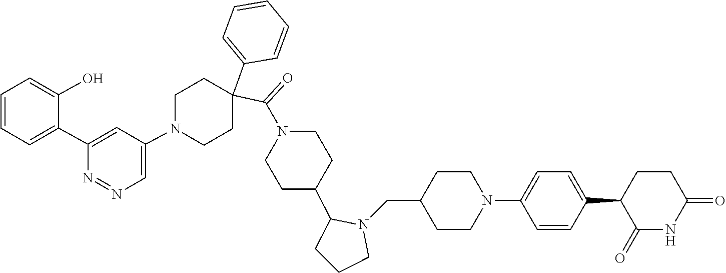 [SMILES: O=C1CC[C@H](c2ccc(N3CCC(CN4CCCC4C4CCN(C(=O)C5(c6ccccc6)CCN(c6cnnc(-c7ccccc7O)c6)CC5)CC4)CC3)cc2)C(=O)N1]

### Step 1: Synthesis of 2-(5-{4-phenyl-4-[4-(pyrrolidin-2-yl)piperidine-1-carbonyl]piperidin-1-yl}pyridazin-3-yl)phenol

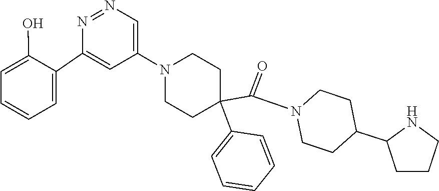 [SMILES: O=C(N1CCC(C2CCCN2)CC1)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added tert-butyl 2-(piperidin-4-yl)pyrrolidine-1-carboxylate (44.82 mg, 0.1762 mmol) and stirred at room temperature until complete by LCMS. Directly injected on RP-FC and purified with a gradient of 5-80% MeCN in H₂O to yield the Boc-protected intermediate (17.2 mg, 22%). Dissolved this material in 1:1 TFA:DCM and stirred for 1 h at room temperature, then concentrated and lyophilized to use in the next step. LCMS: C₃H₃₇N₅O₂ requires: 511.3. found: m/z=512.3 [M+H]⁺.

### Step 2: Synthesis of the Title Compound

Dissolved 2-(5-{4-phenyl-4-[4-(pyrrolidin-2-yl)piperidine-1-carbonyl]piperidin-1-yl}pyridazin-3-yl)phenol (16.30 mg, 0.0319 mmol), 1-{4-[(3R)-2,6-dioxopiperidin-3-yl]phenyl}piperidine-4-carbaldehyde (10.53 mg, 0.0350 mmol), and N,N-diisopropylethylamine (27.82 μL, 20.59 mg, 0.1593 mmol) in DCE and stirred for 30 min at room temperature. Then added sodium triacetoxyborohydride (20.25 mg, 0.0956 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (11.3 mg, 45%). LCMS: C₄₈H₅₇N₇O₄ requires: 795.4. found: m/z=796.5 [M+H]⁺.

### Example 41

### (3R)-3-(4-{4-[(5-{1-[6-(2-hydroxyphenyl)pyridazin-4-yl]-4-phenylpiperidine-4-carbonyl}-decahydro-1,5-naphthyridin-1-yl)methyl]piperidin-1-yl}phenyl)piperidine-2,6-dione

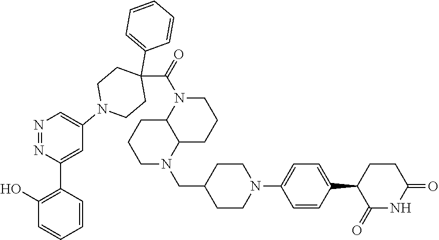 [SMILES: O=C1CC[C@H](c2ccc(N3CCC(CN4CCCC5C4CCCN5C(=O)C4(c5ccccc5)CCN(c5cnnc(-c6ccccc6O)c5)CC4)CC3)cc2)C(=O)N1]

### Step 1: Synthesis of 2-{5-[4-(octahydro-2H-1,5-naphthyridine-1-carbonyl)-4-phenylpiperidin-1-yl]pyridazin-3-yl}phenol

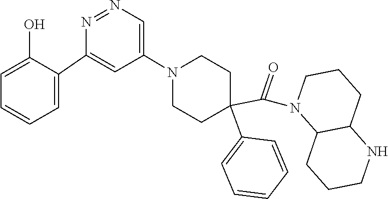 [SMILES: O=C(N1CCCC2NCCCC21)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added tert-butyl octahydro-2H-1,5-naphthyridine-1-carboxylate (42.35 mg, 0.1762 mmol) and stirred at room temperature until complete by LCMS. Directly injected on RP-FC and purified with a gradient of 5-80% MeCN in H₂O to yield the Boc-protected intermediate (17.2 mg, 22%). Dissolved this material in 1:1 TFA:DCM and stirred for 1 h at room temperature, then concentrated and lyophilized to use in the next step. LCMS: C₃₀H₃₅N₅O₂ requires: 497.3. found: m/z=498.3 [M+H]⁺.

### Step 2: Synthesis of the Title Compound

Dissolved 2-{5-[4-(octahydro-2H-1,5-naphthyridine-1-carbonyl)-4-phenylpiperidin-1-yl]pyridazin-3-yl}phenol (22.30 mg, 0.0448 mmol), 1-{4-[(3R)-2,6-dioxopiperidin-3-yl]phenyl}piperidine-4-carbaldehyde (14.81 mg, 0.0493 mmol), and N,N-diisopropylethylamine (39.13 μL, 28.96 mg, 0.2241 mmol) in DCE and stirred for 30 min at room temperature. Then added sodium triacetoxyborohydride (28.49 mg, 0.1344 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (15.2 mg, 43%). LCMS: C₄₇H₅₅N₇O₄ requires: 781.4. found: m/z=782.4 [M+H]⁺.

### Example 42

### (3R)-3-[4-(4-{[2-(1-{1-[6-(2-hydroxyphenyl)pyridazin-4-yl]-4-phenylpiperidine-4-carbonyl}piperidin-2-yl)pyrrolidin-1-yl]methyl}piperidin-1-yl)phenyl]piperidine-2,6-dione

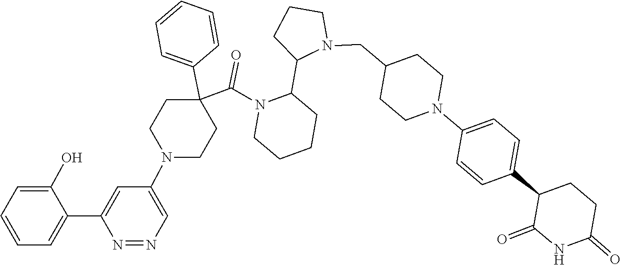 [SMILES: O=C1CC[C@H](c2ccc(N3CCC(CN4CCCC4C4CCCCN4C(=O)C4(c5ccccc5)CCN(c5cnnc(-c6ccccc6O)c5)CC4)CC3)cc2)C(=O)N1]

### Step 1: Synthesis of 2-(5-{4-phenyl-4-[2-(pyrrolidin-2-yl)piperidine-1-carbonyl]piperidin-1-yl}pyridazin-3-yl)phenol

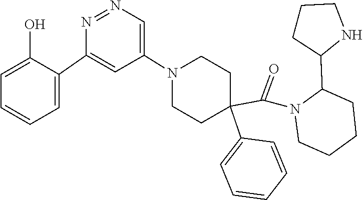 [SMILES: O=C(N1CCCCC1C1CCCN1)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added tert-butyl 2-(piperidin-2-yl)pyrrolidine-1-carboxylate (44.82 mg, 0.1762 mmol) and stirred at room temperature until complete by LCMS. Directly injected on RP-FC and purified with a gradient of 5-80% MeCN in H₂O to yield the Boc-protected intermediate (50.1 mg, 63%). Dissolved this material in 1:1 TFA:DCM and stirred for 1 h at room temperature, then concentrated and lyophilized to use in the next step. LCMS: C₃₁H₃₇N₅O₂ requires: 511.3. found: m/z=512.3 [M+H]⁺.

### Step 2: Synthesis of

Dissolved 2-(5-{4-phenyl-4-[2-(pyrrolidin-2-yl)piperidine-1-carbonyl]piperidin-1-yl}pyridazin-3-yl)phenol (3.70 mg, 0.0072 mmol), 1-{4-[(3R)-2,6-dioxopiperidin-3-yl]phenyl}piperidine-4-carbaldehyde (2.39 mg, 0.0080 mmol), and N,N-diisopropylethylamine (6.31 L, 4.67 mg, 0.0362 mmol) in DCE and stirred for 30 min at room temperature. Then added sodium triacetoxyborohydride (4.60 mg, 0.0217 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (3.3 mg, 52%). LCMS: C₄₈H₅₇N₇O₄ requires: 795.4. found: m/z=796.5 [M+H]⁺.

### Example 43

### rac-(3R)-3-(4-{4-[(6-{1-[6-(2-hydroxyphenyl)pyridazin-4-yl]-4-(3-methylphenyl)piperidine-4-carbonyl}-2,6-diazaspiro[3.3]heptan-2-yl)methyl]piperidin-1-yl}phenyl)piperidine-2,6-dione

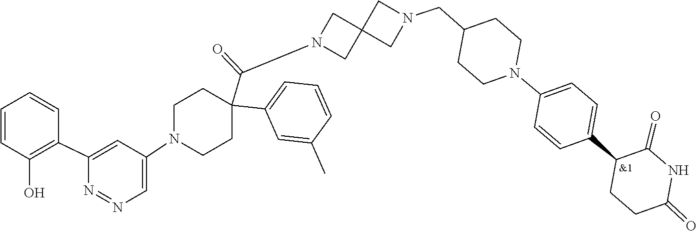 [SMILES: Cc1cccc(C2(C(=O)N3CC4(CN(CC5CCN(c6ccc([C@@H]7CCC(=O)NC7=O)cc6)CC5)C4)C3)CCN(c3cnnc(-c4ccccc4O)c3)CC2)c1]

### Step 1: Synthesis of 1-(tert-butyl) 4-methyl 4-(m-tolyl)piperidine-1,4-dicarboxylate

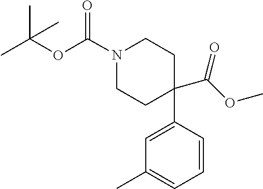 [SMILES: COC(=O)C1(c2cccc(C)c2)CCN(C(=O)OC(C)(C)C)CC1]

To a solution of 1-(tert-butyl) 4-methyl 4-(m-tolyl)piperidine-1,4-dicarboxylate (10.0 g, 59.6 mmol, 1.00 eq) in DMF (100 mL) was added NaH (5.97 g, 149 mmol, 60% purity, 2.50 eq) at 0-5° C. under N₂, and the mixture was stirred at 0-5° C. for 1 hr. Then tert-butyl bis(2-chloroethyl)carbamate (15.9 g, 65.6 mmol, 1.10 eq) was added and the mixture was heated to 60° C. and stirred for 3 hrs. The mixture was cooled down to 25° C., then poured into saturated aqueous NH₄Cl (200 mL) solution, then extracted with EtOAc (2×100 mL). The combined organic layers were washed with brine (2×100 mL), dried over Na₂SO₄, filtered, and concentrated to give the title compound as a yellow oil (24.2 g, crude). LCMS: C₁₉H₂₇NO₄ requires: 333.2. found: m/z=234.2 [M−100+H]⁺.

### Step 2: Synthesis of methyl 4-(m-tolyl)piperidine-4-carboxylate

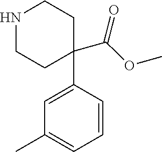 [SMILES: COC(=O)C1(c2cccc(C)c2)CCNCC1]

A solution of 1-(tert-butyl) 4-methyl 4-(m-tolyl)piperidine-1,4-dicarboxylate (24.0 g, crude) in HCl/EtOAc (4.00 M, 100 mL) was stirred at 20-25° C. for 12 hrs. The reaction mixture was filtered and the residue was collected. The title compound was obtained as an off-white solid (6.21 g, 22.0 mmol, 30.6% yield, 95.9% purity, HCl). LCMS: C₁₄H₁₉NO₂ requires: 233.1. found: m/z=234.2 [M+H]⁺.

### Step 3: Synthesis of methyl 1-(6-chloropyridazin-4-yl)-4-(m-tolyl)piperidine-4-carboxylate

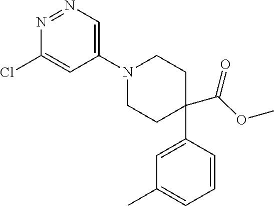 [SMILES: COC(=O)C1(c2cccc(C)c2)CCN(c2cnnc(Cl)c2)CC1]

To a solution of methyl 4-(m-tolyl)piperidine-4-carboxylate (5.00 g, 17.7 mmol, 1.00 eq, HCl) in IPA (50.0 mL) was added DIPEA (9.19 g, 71.1 mmol, 12.3 mL, 4.00 eq) and 3,5-dichloropyridazine (3.18 g, 21.3 mmol, 1.20 eq) at 20-25° C., the mixture was heated to 70° C. and stirred for 2 hrs. The mixture was concentrated, then water was added (100 mL) and extracted with DCM (3×50.0 mL). The organic layers were washed with brine (2×100 mL), dried over Na₂SO₄, filtered, and concentrated. The residue was purified by column chromatography (SiO₂, Petroleum ether/Ethyl acetate=I/O to 2/1, Petroleum ether/Ethyl acetate=0/1, R_{f}=0.60) to furnish the title compound as a yellow solid (3.50 g, 9.78 mmol, 55.0% yield, 96.6% purity). LCMS: C₁₈H₂₀ClN₃O₂ requires: 345.1. found: m/z=346.2 [M+H]⁺. ¹H NMR: (400 MHz, CDCl₃) δ 8.68 (d, J=2.4 Hz, 1H), 7.19-7.10 (m, 1H), 7.08 (d, J=2.4 Hz, 2H), 7.04-7.02 (m, 1H), 6.61 (d, J=2.4 Hz, 1H), 3.75-3.70 (m, 2H), 3.63 (s, 3H), 3.14 (br t, J=11.2 Hz, 2H), 2.60 (br d, J=2.4 Hz, 2H), 2.28 (s, 3H), 1.99-1.92 (m, 2H).

### Step 4: Synthesis of methyl 1-(6-(2-hydroxyphenyl)pyridazin-4-yl)-4-(m-tolyl)piperidine-4-carboxylate

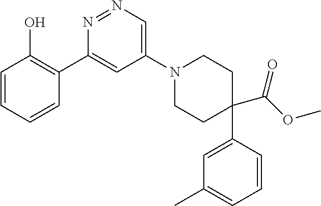 [SMILES: COC(=O)C1(c2cccc(C)c2)CCN(c2cnnc(-c3ccccc3O)c2)CC1]

To a solution of methyl 1-(6-chloropyridazin-4-yl)-4-(m-tolyl)piperidine-4-carboxylate (3.50 g, 9.78 mmol, 1.00 eq) in dioxane (30.0 mL) and H₂O (3.00 mL) was added K₂CO₃ (6.76 g, 48.8 mmol, 5.00 eq), (2-hydroxyphenyl)boronic acid (2.70 g, 19.5 mmol, 2.00 eq) and Pd(PPh₃)₂Cl₂ (1.03 g, 1.47 mmol, 0.150 eq) under N₂ atmosphere at 20-25° C. The reaction mixture was stirred at 100° C. for 12 hrs. The reaction mixture was concentrated under vacuum, then was diluted with water (80.0 mL), extracted with DCM (2×20.0 mL). The combined organic layers were washed with brine (2×20.0 mL), dried over Na₂SO₄, filtered, and concentrated under reduced pressure to give the crude. The crude product was triturated with EtOAc (20.0 mL) at 25° C. for 10 mins, then was filtered and the residue was collected. The title compound was obtained as a gray solid (2.35 g, 5.49 mmol, 56.1% yield, 94.3% purity). LCMS: C₂₄H₂₅N₃O₃ requires: 403.2. found: m/z=404.3 [M+H]⁺. ¹H NMR: (400 MHz, CDCl₃) δ 8.74 (d, J=2.8 Hz, 1H), 7.67 (dd, J=1.6, 8.0 Hz, 1H), 7.37-7.31 (m, 1H), 7.30-7.27 (m, 1H), 7.25 (s, 1H), 7.22-7.15 (m, 3H), 7.10 (dd, J=8.0, 16.8 Hz, 2H), 6.97-6.88 (m, 1H), 4.02-3.84 (m, 2H), 3.75-3.70 (m, 3H), 3.40-3.18 (m, 2H), 2.72 (br d, J=13.2 Hz, 2H), 2.38 (s, 3H), 2.16-2.03 (m, 2H).

### Step 5: Synthesis of 1-(6-(2-hydroxyphenyl)pyridazin-4-yl)-4-(m-tolyl)piperidine-4-carboxylic acid

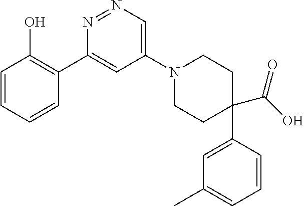 [SMILES: Cc1cccc(C2(C(=O)O)CCN(c3cnnc(-c4ccccc4O)c3)CC2)c1]

To a solution of methyl 1-(6-(2-hydroxyphenyl)pyridazin-4-yl)-4-(m-tolyl)piperidine-4-carboxylate (2.35 g, 5.49 mmol, 1.00 eq) in THF (10.0 mL), MeOH (10.0 mL) and H₂O (5.00 mL) was added NaOH (2.20 g, 54.9 mmol, 10.0 eq) at 20-25° C. The reaction mixture was stirred at 80° C. for 12 hrs. The reaction mixture was evaporated under reduced pressure to remove the solvent, then the mixture was adjusted to pH=3 with 4 N HCl, filtered, and the precipitate was collected.

The crude product was purified by prep-HPLC (column: Phenomenex luna C18 150*40 mm*15 um; mobile phase: [water (HCl)-ACN]; gradient: 20%-50% B over 10 min). The title compound was obtained as a white solid (504 mg, 1.17 mmol, 21.3% yield, 99.0% purity, HCl). LCMS: C₂₃H₂₃N₃O₃ requires: 389.2. found: m/z=390.2 [M+H]⁺. ¹H NMR: (400 MHz, MeOD) δ 8.85 (d, J=3.2 Hz, 1H), 7.67 (d, J=7.2 Hz, 1H), 7.48-7.41 (m, 2H), 7.29-7.22 (m, 3H), 7.10 (br d, J=8.0 Hz, 1H), 7.05-7.00 (m, 2H), 4.26 (br d, J=14.0 Hz, 2H), 3.52 (br t, J=12.0 Hz, 2H), 2.72 (br d, J=14.0 Hz, 2H), 2.35 (s, 3H), 2.13-2.06 (m, 2H).

### Step 6: Synthesis of the title compound

1-[6-(2-hydroxyphenyl)pyridazin-4-yl]-4-(pyridin-3-yl)piperidine-4-carboxylic acid (20.00 mg, 0.0450 mmol), PyBOP; hexafluoro-lambda5-phosphanuide (35.14 mg, 0.0675 mmol), and N,N-diisopropylethylamine (39.31 μL, 29.09 mg, 0.2251 mmol) were stirred in DMF for 1 hour at room temperature. rac-(3R)-3-[4-(4-{2,6-diazaspiro[3.3]heptan-2-ylmethyl}piperidin-1-yl)phenyl]piperidine-2,6-dione (22.38 mg, 0.0585 mmol) was then added and the reaction was stirred overnight. The solution was injected onto RP-FC and purified with a gradient of 0-70% MeCN in H₂O to furnish the title compound (7.7 mg, 21%). LCMS: C₄₅H₅₁N₇O₄ requires: 753.4, found: m/z=754.4 [M+H]⁺.

### Example 44

### rac-(3R)-3-[4-(4-{[6-({-[6-(2-hydroxyphenyl)pyridazin-4-yl]-4-phenylpiperidin-4-yl}methyl)-2,6-diazaspiro[3.3]heptan-2-yl]methyl}piperidin-1-yl)phenyl]piperidine-2,6-dione

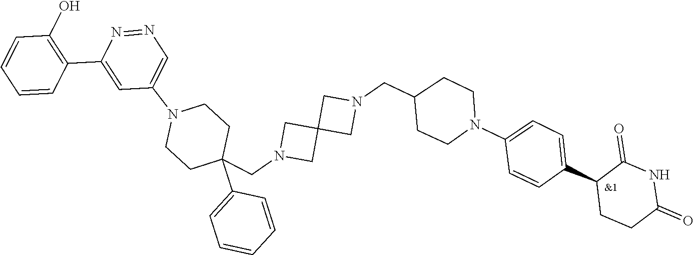 [SMILES: O=C1CC[C@@H](c2ccc(N3CCC(CN4CC5(C4)CN(CC4(c6ccccc6)CCN(c6cnnc(-c7ccccc7O)c6)CC4)C5)CC3)cc2)C(=O)N1]

### Step 1: Synthesis of 2-(5-(4-(hydroxymethyl)-4-phenylpiperidin-1-yl)pyridazin-3-yl)phenol

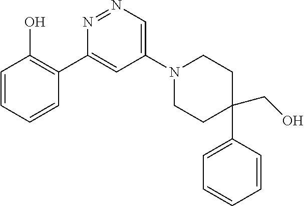 [SMILES: OCC1(c2ccccc2)CCN(c2cnnc(-c3ccccc3O)c2)CC1]

To a solution of methyl 1-(6-(2-hydroxyphenyl)pyridazin-4-yl)-4-phenylpiperidine-4-carboxylate (1.50 g, 3.85 mmol, 1.00 eq) in THF (30.0 mL) and DCM (10.0 mL) was added LAH (219 mg, 5.78 mmol, 1.50 eq) at 0° C. The mixture was stirred at 25° C. for 12 hrs, then cooled to 0° C. Slowly added water (20.0 mL) followed by 15% sodium hydroxide aqueous solution (10.0 mL) and additional water (20.0 mL). Warmed to room temperature and stirred for 15 minutes. Added MgSO₄, stirred another 15 minutes, filtered, and concentrated under vacuum to yield the title compound as a yellow solid (1.38 g, 3.72 mmol, 96.5% yield, 97.4% purity). LCMS: C₂₂H₂₃N₃O₂ requires: 361.2. found: m/z=362.4 [M+H]⁺. ¹H NMR: (400 MHz, DMSO) δ 14.6 (s, 1H), 8.92 (d, J=2.8 Hz, 1H), 8.09 (dd, J=1.6, 8.4 Hz, 1H), 7.51 (d, J=2.8 Hz, 1H), 7.46-7.41 (m, 2H), 7.38-7.34 (m, 1H), 7.39-7.33 (m, 3H), 7.32-7.29 (m, 1H), 7.25-7.18 (m, 1H), 6.96-6.89 (m, 2H), 4.72 (t, J=5.2 Hz, 1H), 3.95 (td, J=4.0, 13.2 Hz, 2H), 3.41 (d, J=5.2 Hz, 2H), 3.23-3.10 (m, 2H), 2.19 (br d, J=14.2 Hz, 2H), 1.99 (s, 1H).

### Step 2: Synthesis of 2-(5-(4-formyl-4-phenylpiperidin-1-yl)pyridazin-3-yl)phenyl hydrogen sulfate

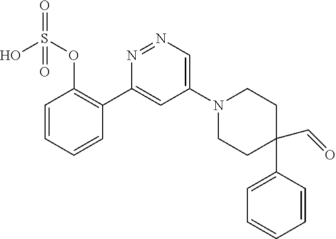 [SMILES: O=CC1(c2ccccc2)CCN(c2cnnc(-c3ccccc3OS(=O)(=O)O)c2)CC1]

To a solution of 2-(5-(4-(hydroxymethyl)-4-phenylpiperidin-1-yl)pyridazin-3-yl)phenol (1.54 g, 4.15 mmol, 1.00 eq) in DCM (15.4 mL) was added DIEA (1.61 g, 12.4 mmol, 2.17 mL, 3.00 eq) and DMSO (972 mg, 12.4 mmol, 972 μL, 3.00 eq). The mixture was cooled to 0° C., then added Py·O₃ (1.98 g, 12.4 mmol, 3.00 eq). The mixture was stirred at 20° C. for 1 hr. The reaction mixture was filtered, the filter cake was soaked in water, and the filtrate was concentrated under vacuum. The crude product was purified by reverse-phase chromatography (water(NH₄HCO₃)—ACN condition), separation gradient: 3%-33%, B over 20 min. The title compound was obtained as a white solid (1.60 g, 3.64 mmol, 87.7% yield, 100% purity). LCMS: C₂₂H₂₁N₃O₅S requires: 439.1, found: m/z=440.2 [M+H]⁺.

### Step 3: Synthesis of 1-(6-(2-hydroxyphenyl)pyridazin-4-yl)-4-phenylpiperidine-4-carbaldehyde

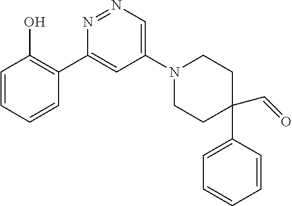 [SMILES: O=CC1(c2ccccc2)CCN(c2cnnc(-c3ccccc3O)c2)CC1]

To a solution of 2-(5-(4-formyl-4-phenylpiperidin-1-yl)pyridazin-3-yl)phenyl hydrogen sulfate (16.0 g, 3.48 mmol, 1.00 eq) in H₂O (65.0 mL) and ACN (35.0 mL) was added HCl (12.0 M, 26.7 mL, 92.2 eq) at 20° C. The mixture was stirred at 50° C. for 2 hrs. The reaction mixture was adjusted to pH=6 with NaHCO₃ and extracted with ethyl acetate (3×20.0 mL). The combined organic layers were washed with brine (20.0 mL), dried over Na₂SO₄, filtered, and concentrated under reduced pressure to give a residue. The crude product was purified by re-crystallization from ethyl acetate (30.0 mL) at 20° C. for 1 hr to yield the title compound as a yellow solid (467.03 mg, 1.29 mmol, 37.07% yield, 99.3% purity). LCMS: C₂₂H₂₁N₃O₂ requires: 359.2. found: m/z=360.3 [M+H]⁺. ¹H NMR (400 MHz, DMSO) δ 14.57 (br s, 1H), 9.56 (s, 1H), 8.97 (d, J=2.8 Hz, 1H), 8.14-8.08 (m, 1H), 7.57 (d, J=2.8 Hz, 1H), 7.47-7.37 (m, 5H), 7.35-7.29 (m, 2H), 6.95-6.88 (m, 2H), 4.13-4.00 (m, 2H), 3.37-3.35 (m, 1H), 3.29 (br d, J=2.4 Hz, 1H), 2.54 (br s, 2H), 2.12-2.01 (m, 2H).

### Step 4: Synthesis of the title compound

Dissolved 1-[6-(2-hydroxyphenyl)pyridazin-4-yl]-4-phenylpiperidine-4-carbaldehyde (25.00 mg, 0.0696 mmol), rac-(3R)-3-[4-(4-{2,6-diazaspiro[3.3]heptan-2-ylmethyl}piperidin-1-yl)phenyl]piperidine-2,6-dione (34.59 mg, 0.0904 mmol), and N,N-diisopropylethylamine (60.74 L, 44.95 mg, 0.3478 mmol) in DCE and stirred for 30 min at room temperature. Then added sodium triacetoxyborohydride (44.22 mg, 0.2087 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (13.1 mg, 26%). LCMS: C₄₄H₅₁N₇O₃ requires: 725.4. found: m/z=726.4 [M+H]⁺.

### Example 45

### (3R)-3-(4-{4-[(9-{1-[6-(2-hydroxyphenyl)pyridazin-4-yl]-4-phenylpiperidine-4-carbonyl}-3,9-diazabicyclo[3.3.2]decan-3-yl)methyl]piperidin-1-yl}phenyl)piperidine-2,6-dione

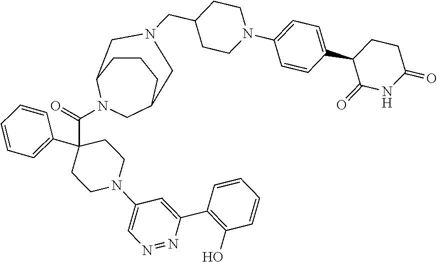 [SMILES: O=C1CC[C@H](c2ccc(N3CCC(CN4CC5CCCC(C4)N(C(=O)C4(c6ccccc6)CCN(c6cnnc(-c7ccccc7O)c6)CC4)C5)CC3)cc2)C(=O)N1]

### Step 1: Synthesis of 2-[5-(4-{3-benzyl-3,9-diazabicyclo[3.3.2]decane-9-carbonyl}-4-phenylpiperidin-1-yl)pyridazin-3-yl]phenol

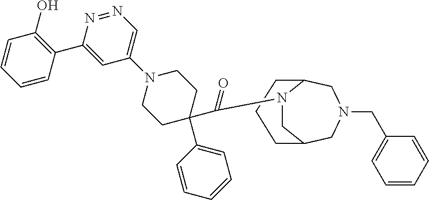 [SMILES: O=C(N1CC2CCCC1CN(Cc1ccccc1)C2)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added 3-benzyl-3,9-diazabicyclo[3.3.2]decane (40.59 mg, 0.1762 mmol) and stirred at room temperature until complete by LCMS. The solution was injected onto RP-FC and purified with a gradient of 0-70% MeCN in H₂O to furnish the title compound (20.0 mg, 23%). LCMS: C₃₇H₄₁N₅O₂ requires: 587.3. found: m/z=588.3 [M+H]⁺.

### Step 2: Synthesis of 2-[5-(4-{3,9-diazabicyclo[3.3.2]decane-9-carbonyl}-4-phenylpiperidin-1-yl)pyridazin-3-yl]phenol

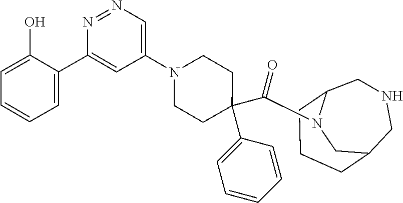 [SMILES: O=C(N1CC2CCCC1CNC2)C1(c2ccccc2)CCN(c2cnnc(-c3ccccc3O)c2)CC1]

2-[5-(4-{3-benzyl-3,9-diazabicyclo[3.3.2]decane-9-carbonyl}-4-phenylpiperidin-1-yl)pyridazin-3-yl]phenol (20.00 mg, 0.0340 mmol) and palladium on carbon (0.3 mg, 0.0003 mmol) was dissolved in dry MeOH and N₂ was bubbled through the solution using a balloon for 5 minutes.

The balloon was then switched to H₂ and bubbled a further 5 minutes before being left to stir under H₂ atmosphere overnight. The palladium was removed by celite filtration to yield the crude product in quantitative yield, which was used in the next step without additional purification. LCMS: C₃₀H₃₅N₅O₂ requires: 497.3. found: m/z=498.6 [M+H]⁺.

### Step 3: Synthesis of the Title Compound

Dissolved 2-[5-(4-{3,9-diazabicyclo[3.3.2]decane-9-carbonyl}-4-phenylpiperidin-1-yl)pyridazin-3-yl]phenol (3.70 mg, 0.0072 mmol), 1-{4-[(3R)-2,6-dioxopiperidin-3-yl]phenyl}piperidine-4-carbaldehyde (2.39 mg, 0.0080 mmol), and N,N-diisopropylethylamine (6.31 L, 4.67 mg, 0.0362 mmol) in DCE and stirred for 30 min at room temperature. Then added sodium triacetoxyborohydride (4.60 mg, 0.0217 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (0.5 mg, 8%). LCMS: C₄₇H₅₅N₇O₄ requires: 781.4. found: m/z=782.4 [M+H]⁺.

### Example 46

### (3R)-3-(4-{4-[(4-{1-[6-(2-hydroxyphenyl)pyridazin-4-yl]-4-phenylpiperidine-4-carbonyl}-1,4-diazepan-1-yl)methyl]piperidin-1-yl}phenyl)piperidine-2,6-dione

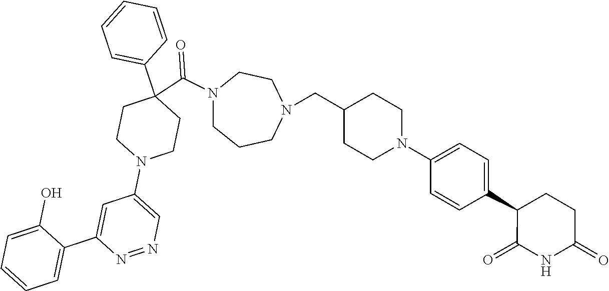 [SMILES: O=C1CC[C@H](c2ccc(N3CCC(CN4CCCN(C(=O)C5(c6ccccc6)CCN(c6cnnc(-c7ccccc7O)c6)CC5)CC4)CC3)cc2)C(=O)N1]

### Step 1: Synthesis of 2-{5-[4-(4-benzyl-1,4-diazepane-1-carbonyl)-4-phenylpiperidin-1-yl]pyridazin-3-yl}phenol

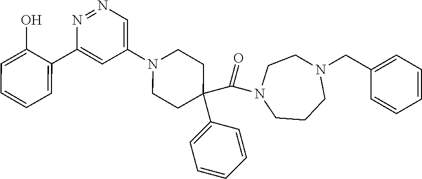 [SMILES: O=C(N1CCCN(Cc2ccccc2)CC1)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added 3-benzyl-3,9-diazabicyclo[3.3.2]decane (40.59 mg, 0.1762 mmol) and stirred at room temperature until complete by LCMS. The solution was injected onto RP-FC and purified with a gradient of 0-70% MeCN in H₂O to furnish the title compound (23.3 mg, 30%). LCMS: C₃₄H₃₇N₅O₂ requires: 547.3. found: m/z=548.3 [M+H]⁺.

### Step 2: Synthesis of 2-{5-[4-(1,4-diazepane-1-carbonyl)-4-phenylpiperidin-1-yl]pyridazin-3-yl}phenol

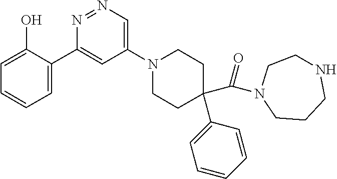 [SMILES: O=C(N1CCCNCC1)C1(c2ccccc2)CCN(c2cnnc(-c3ccccc3O)c2)CC1]

2-{5-[4-(4-benzyl-1,4-diazepane-1-carbonyl)-4-phenylpiperidin-1-yl]pyridazin-3-yl}phenol (20.00 mg, 0.0340 mmol) and palladium on carbon (0.3 mg, 0.0003 mmol) were dissolved in dry MeOH and N₂ was bubbled through the solution using a balloon for 5 minutes. The balloon was then switched to H₂ and bubbled a further 5 minutes before being left to stir under H₂ atmosphere overnight. The palladium was removed by celite filtration to yield the crude product in quantitative yield, which was used in the next step without additional purification. LCMS: C₂₇H₃₁N₅O₂ requires: 457.2. found: m/z=458.3 [M+H]⁺.

### Step 3: Synthesis of the Title Compound

Dissolved 2-{5-[4-(1,4-diazepane-1-carbonyl)-4-phenylpiperidin-1-yl]pyridazin-3-yl}phenol (15.00 mg, 0.0328 mmol), 1-{4-[(3R)-2,6-dioxopiperidin-3-yl]phenyl}piperidine-4-carbaldehyde (10.83 mg, 0.0361 mmol), and N,N-diisopropylethylamine (28.63 μL, 21.18 mg, 0.1639 mmol) in DCE and stirred for 30 min at room temperature. Then added sodium triacetoxyborohydride (20.84 mg, 0.0983 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (10.5 mg, 44%). LCMS: C₄₄H₅₁N₇O₄ requires: 741.4. found: m/z=742.5 [M+H]⁺.

### Example 47

### N-{2-[(1-{4-[(3R)-2,6-dioxopiperidin-3-yl]phenyl}piperidin-4-yl)methyl]-2-azabicyclo[2.2.1]heptan-6-yl}-1-[6-(2-hydroxyphenyl)pyridazin-4-yl]-4-phenylpiperidine-4-carboxamide

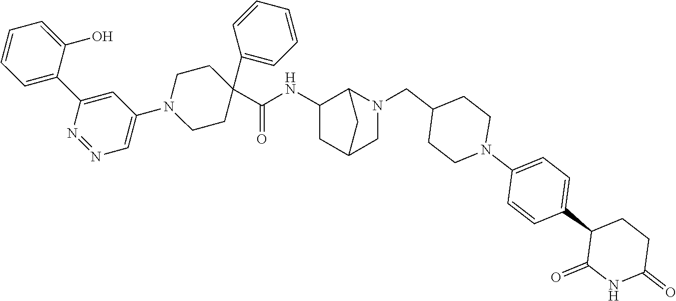 [SMILES: O=C1CC[C@H](c2ccc(N3CCC(CN4CC5CC(NC(=O)C6(c7ccccc7)CCN(c7cnnc(-c8ccccc8O)c7)CC6)C4C5)CC3)cc2)C(=O)N1]

### Step 1: Synthesis of N-{2-azabicyclo[2.2.1]heptan-6-yl}-1-[6-(2-hydroxyphenyl)pyridazin-4-yl]-4-phenylpiperidine-4-carboxamide

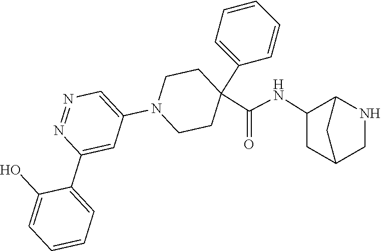 [SMILES: O=C(NC1CC2CNC1C2)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (50.00 mg, 0.1332 mmol), [(dimethylamino)({[1,2,3]triazolo[4,5-b]pyridin-3-yloxy})methylidene]dimethylazanium; hexafluoro-lambda5-phosphanuide (126.60 mg, 0.3330 mmol), and N,N-diisopropylethylamine (116.31 μL, 86.07 mg, 0.6659 mmol) in DMF. Added tert-butyl 6-amino-2-azabicyclo[2.2.1]heptane-2-carboxylate (36.76 mg, 0.1731 mmol) and stirred at room temperature until complete by LCMS. Directly injected on RP-FC and purified with a gradient of 5-80% MeCN in H2O to yield the Boc-protected intermediate (30 mg, 40%). Dissolved this material in 1:1 TFA:DCM and stirred for 1 h at room temperature, then concentrated and lyophilized to use in the next step. LCMS: C₂₈H₃₁N₅O₂ requires: 469.2. found: m/z=470.3 [M+H]⁺.

### Step 2: Synthesis of the Title Compound

Dissolved N-{2-azabicyclo[2.2.1]heptan-6-yl}-1-[6-(2-hydroxyphenyl)pyridazin-4-yl]-4-phenylpiperidine-4-carboxamide (27.00 mg, 0.0575 mmol), 1-{4-[(3R)-2,6-dioxopiperidin-3-yl]phenyl}piperidine-4-carbaldehyde (19.00 mg, 0.0632 mmol), and N,N-diisopropylethylamine (50.21 μL, 37.16 mg, 0.2875 mmol) in DCE and stirred for 30 min at room temperature. Then added sodium triacetoxyborohydride (36.56 mg, 0.1725 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (16.2 mg, 34%). LCMS: C₄₅H₅₁N₇O₄ requires: 753.4. found: m/z=754.3 [M+H]⁺.

### Example 48

### (3R)-3-[4-(4-{[3-(1-{1-[6-(2-hydroxyphenyl)pyridazin-4-yl]-4-phenylpiperidine-4-carbonyl}piperidin-4-yl)pyrrolidin-1-yl]methyl}piperidin-1-yl)phenyl]piperidine-2,6-dione

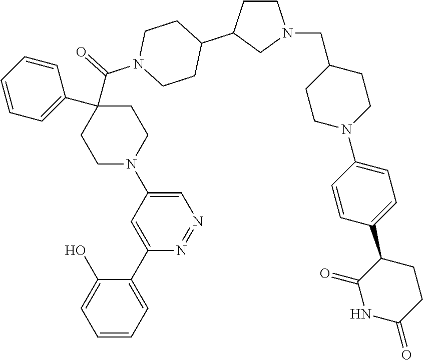 [SMILES: O=C1CC[C@H](c2ccc(N3CCC(CN4CCC(C5CCN(C(=O)C6(c7ccccc7)CCN(c7cnnc(-c8ccccc8O)c7)CC6)CC5)C4)CC3)cc2)C(=O)N1]

### Step1: Synthesis of 2-(5-{4-phenyl-4-[4-(pyrrolidin-3-yl)piperidine-1-carbonyl]piperidin-1-yl}pyridazin-3-yl)phenol

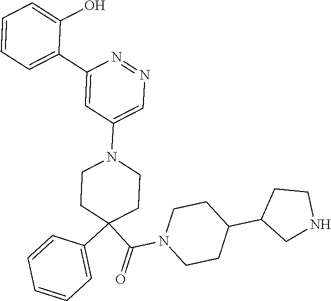 [SMILES: O=C(N1CCC(C2CCNC2)CC1)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added tert-butyl 3-(piperidin-4-yl)pyrrolidine-1-carboxylate (43.16 mg, 0.1697 mmol) and stirred at room temperature until complete by LCMS. Directly injected on RP-FC and purified with a gradient of 5-80% MeCN in H2O to yield the Boc-protected intermediate (31 mg, 39%). Dissolved this material in 1:1 TFA:DCM and stirred for 1 h at room temperature, then concentrated and lyophilized to use in the next step. LCMS: C₃₁H₃₇N₅O₂ requires: 511.3. found: m/z=512.3 [M+H]⁺.

### Step 2: Synthesis of the title compound

Dissolved 2-(5-{4-phenyl-4-[4-(pyrrolidin-3-yl)piperidine-1-carbonyl]piperidin-1-yl}pyridazin-3-yl)phenol (29.00 mg, 0.0567 mmol), 1-{4-[(3R)-2,6-dioxopiperidin-3-yl]phenyl}piperidine-4-carbaldehyde (18.73 mg, 0.0623 mmol), and N,N-diisopropylethylamine (49.50 μL, 36.63 mg, 0.2834 mmol) in DCE and stirred for 30 min at room temperature. Then added sodium triacetoxyborohydride (36.04 mg, 0.1700 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (19.0 mg, 42%). LCMS: C₄₈H₅₇N₇O₄ requires: 795.4. found: m/z=796.5 [M+H]⁺.

### Example 49

### N-(3-{[(1-{4-[(3R)-2,6-dioxopiperidin-3-yl]phenyl}piperidin-4-yl)methyl]amino}bicyclo[3.3.1]nonan-9-yl)-1-[6-(2-hydroxyphenyl)pyridazin-4-yl]-4-phenylpiperidine-4-carboxamide

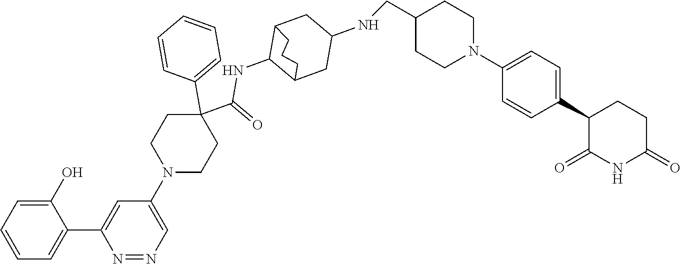 [SMILES: O=C1CC[C@H](c2ccc(N3CCC(CNC4CC5CCCC(C4)C5NC(=O)C4(c5ccccc5)CCN(c5cnnc(-c6ccccc6O)c5)CC4)CC3)cc2)C(=O)N1]

### Step 1: Synthesis of N-{3-aminobicyclo[3.3.1]nonan-9-yl}-1-[6-(2-hydroxyphenyl)pyridazin-4-yl]-4-phenylpiperidine

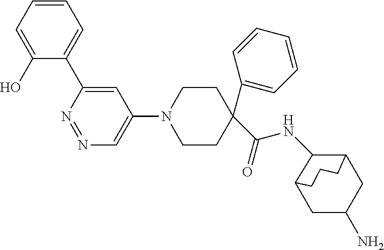 [SMILES: NC1CC2CCCC(C1)C2NC(=O)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added tert-butyl N-{9-aminobicyclo[3.3.1]nonan-3-yl}carbamate (44.82 mg, 0.1762 mmol) and stirred at room temperature until complete by LCMS. Directly injected on RP-FC and purified with a gradient of 5-80% MeCN in H2O to yield the Boc-protected intermediate (30 mg, 33%). Dissolved this material in 1:1 TFA:DCM and stirred for 1 h at room temperature then concentrated and lyophilized to use in the next step. LCMS: C₃₁H₃₇N₅O₂ requires: 511.3. found: m/z=512.3 [M+H]⁺.

### Step 2: Synthesis of the Title Compound

Dissolved N-{3-aminobicyclo[3.3.1]nonan-9-yl}-1-[6-(2-hydroxyphenyl)pyridazin-4-yl]-4-phenylpiperidine-4-carboxamide (51.00 mg, 0.0997 mmol), 1-{4-[(3R)-2,6-dioxopiperidin-3-yl]phenyl}piperidine-4-carbaldehyde (32.93 mg, 0.1096 mmol), and N,N-diisopropylethylamine (87.04 μL, 64.41 mg, 0.4984 mmol) in DCE and stirred for 30 min at room temperature. Then added sodium triacetoxyborohydride (63.37 mg, 0.2990 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (21.4 mg, 26%). LCMS: C₄₈H₅₇N₇O₄ requires: 795.4. found: m/z=796.4 [M+H]⁺.

### Example 50

### (3R)-3-(4-{4-[(1-{1-[6-(2-hydroxyphenyl)pyridazin-4-yl]-4-phenylpiperidine-4-carbonyl}-2-methyl-hexahydropyrrolo[2,3-c]pyrrol-5-yl)methyl]piperidin-1-yl}phenyl)piperidine-2,6-dione

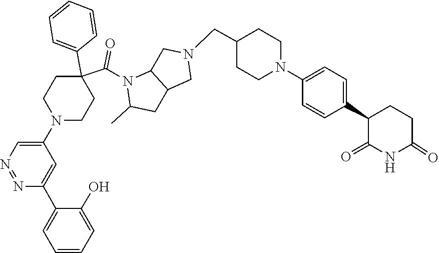 [SMILES: CC1CC2CN(CC3CCN(c4ccc([C@H]5CCC(=O)NC5=O)cc4)CC3)CC2N1C(=O)C1(c2ccccc2)CCN(c2cnnc(-c3ccccc3O)c2)CC1]

### Step 1: Synthesis of 2-[5-(4-{2-methyl-hexahydro-2H-pyrrolo[2,3-c]pyrrole-1-carbonyl}-4-phenylpiperidin-1-yl)pyridazin-3-yl]phenol

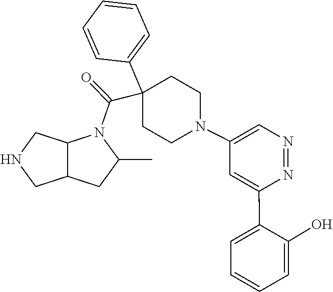 [SMILES: CC1CC2CNCC2N1C(=O)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (50.00 mg, 0.1332 mmol), [(dimethylamino)({[1,2,3]triazolo[4,5-b]pyridin-3-yloxy})methylidene]dimethylazanium; hexafluoro-lambda5-phosphanuide (126.60 mg, 0.3330 mmol), and N,N-diisopropylethylamine (116.31 μL, 86.07 mg, 0.6659 mmol) in DMF. Added tert-butyl 2-methyl-hexahydro-1H-pyrrolo[3,4-b]pyrrole-5-carboxylate (40.69 mg, 0.1798 mmol) and stirred at room temperature until complete by LCMS. Directly injected onto RP-FC and purified using a gradient of 5-80% MeCN in water to yield the Boc-protected intermediate (34 mg, 38%). Dissolved this material in 1:1 TFA:DCM and stirred for 1 h at room temperature, then concentrated and lyophilized to use in the next step. LCMS: C₂₉H₃₃N₅O₂ requires: 483.3. found: m/z=484.3 [M+H]⁺.

### Step 2: Synthesis of the Title Compound

Dissolved 2-[5-(4-{2-methyl-hexahydro-2H-pyrrolo[2,3-c]pyrrole-1-carbonyl}-4-phenylpiperidin-1-yl)pyridazin-3-yl]phenol (7.00 mg, 0.0145 mmol), 1-{4-[(3R)-2,6-dioxopiperidin-3-yl]phenyl}piperidine-4-carbaldehyde (4.78 mg, 0.0159 mmol), and N,N-diisopropylethylamine (12.64 μL, 9.35 mg, 0.0724 mmol) in DCE and stirred for 30 min at room temperature. Then added sodium triacetoxyborohydride (9.20 mg, 0.0434 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (5 mg, 45%). LCMS: C₄₆H₅₃N₇O₄ requires: 767.4. found: m/z=768.3 [M+H]⁺.

### Example 51

### (3R)-3-(4-{4-[(8-{1-[6-(2-hydroxyphenyl)pyridazin-4-yl]-4-phenylpiperidine-4-carbonyl}-3,8-diazabicyclo[4.2.0]octan-3-yl)methyl]piperidin-1-yl}phenyl)piperidine-2,6-dione

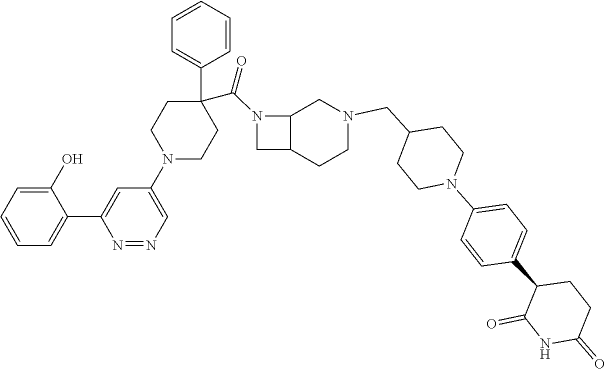 [SMILES: O=C1CC[C@H](c2ccc(N3CCC(CN4CCC5CN(C(=O)C6(c7ccccc7)CCN(c7cnnc(-c8ccccc8O)c7)CC6)C5C4)CC3)cc2)C(=O)N1]

### Step 1: Synthesis of 2-[5-(4-{3,8-diazabicyclo[4.2.0]octane-8-carbonyl}-4-phenylpiperidin-1-yl)pyridazin-3-yl]phenol

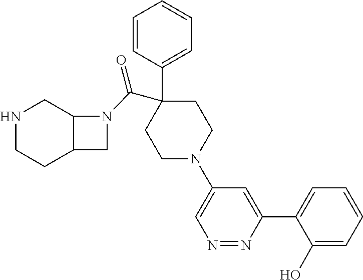 [SMILES: O=C(N1CC2CCNCC21)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added tert-butyl 3,8-diazabicyclo[4.2.0]octane-3-carboxylate (37.41 mg, 0.1762 mmol) and stirred at room temperature until complete by LCMS. Directly injected onto RP-FC and purified using a gradient of 5-80% MeCN in water to yield the Boc-protected intermediate (30 mg, 36%). Dissolved this material in 1:1 TFA:DCM and stirred for 1 h at room temperature, then concentrated and lyophilized to use in the next step. LCMS: C₂₈H₃₁N₅O₂ requires: 469.2. found: m/z=470.3 [M+H]⁺.

### Step 2: Synthesis of (3R)-3-(4-{4-[(8-{1-[6-(2-hydroxyphenyl)pyridazin-4-yl]-4-phenylpiperidine-4-carbonyl}-3,8-diazabicyclo[4.2.0]octan-3-yl)methyl]piperidin-1-yl}phenyl)piperidine-2,6-dione

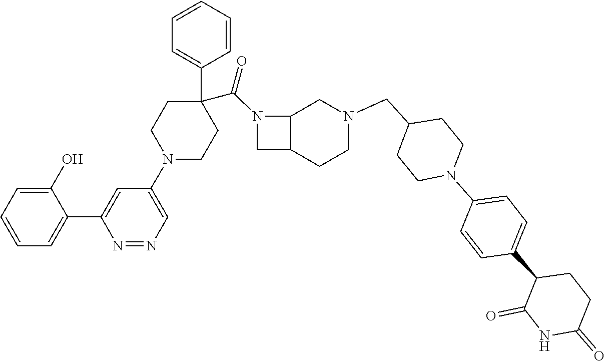 [SMILES: O=C1CC[C@H](c2ccc(N3CCC(CN4CCC5CN(C(=O)C6(c7ccccc7)CCN(c7cnnc(-c8ccccc8O)c7)CC6)C5C4)CC3)cc2)C(=O)N1]

Dissolved 2-[5-(4-{3,8-diazabicyclo[4.2.0]octane-8-carbonyl}-4-phenylpiperidin-1-yl)pyridazin-3-yl]phenol (38.00 mg, 0.0809 mmol), 1-{4-[(3R)-2,6-dioxopiperidin-3-yl]phenyl}piperidine-4-carbaldehyde (26.74 mg, 0.0890 mmol), and N,N-diisopropylethylamine (70.67 μL, 52.29 mg, 0.4046 mmol) in DCE and stirred for 30 min at room temperature. Then added sodium triacetoxyborohydride (51.45 mg, 0.2428 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (22.2 mg, 36%). LCMS: C₄₅H₅₁N₇O₄ requires: 753.4. found: m/z=754.4 [M+H]⁺.

### Example 52

### (3R)-3-(4-{4-[(8,8-difluoro-2-{1-[6-(2-hydroxyphenyl)pyridazin-4-yl]-4-phenylpiperidine-4-carbonyl}-2,6-diazaspiro[3.4]octan-6-yl)methyl]piperidin-1-yl}phenyl)piperidine-2,6-dione

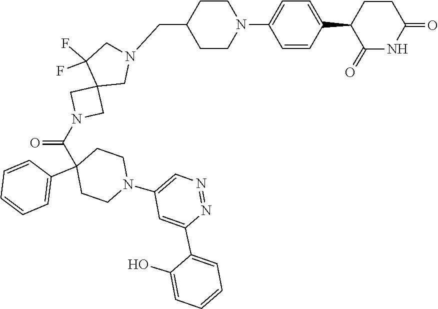 [SMILES: O=C1CC[C@H](c2ccc(N3CCC(CN4CC(F)(F)C5(C4)CN(C(=O)C4(c6ccccc6)CCN(c6cnnc(-c7ccccc7O)c6)CC4)C5)CC3)cc2)C(=O)N1]

### Step 1: Synthesis of 2-[5-(4-{8,8-difluoro-2,6-diazaspiro[3.4]octane-2-carbonyl}-4-phenylpiperidin-1-yl)pyridazin-3-yl]phenol

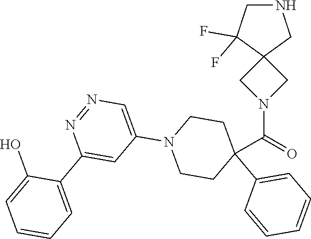 [SMILES: O=C(N1CC2(CNCC2(F)F)C1)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (60.00 mg, 0.1598 mmol), [(dimethylamino)({[1,2,3]triazolo[4,5-b]pyridin-3-yloxy})methylidene]dimethylazanium; hexafluoro-lambda5-phosphanuide (151.92 mg, 0.3995 mmol), and N,N-diisopropylethylamine (139.57 μL, 103.28 mg, 0.7991 mmol) in DMF. Added tert-butyl 8,8-difluoro-2,6-diazaspiro[3.4]octane-6-carboxylate (53.57 mg, 0.2158 mmol) and stirred at room temperature until complete by LCMS. Directly injected onto RP-FC and purified using a gradient of 5-80% MeCN in water to yield the Boc-protected intermediate (80 mg, 74%). Dissolved this material in 1:1 TFA:DCM and stirred for 1 h at room temperature, then concentrated and lyophilized to use in the next step. LCMS: C₂₈H₂₉F₂N₅O₂ requires: 505.2. found: m/z=506.3 [M+H]⁺.

### Step 2: Synthesis of (3R)-3-(4-{4-[(8,8-difluoro-2-{1-[6-(2-hydroxyphenyl)pyridazin-4-yl]-4-phenylpiperidine-4-carbonyl}-2,6-diazaspiro[3.4]octan-6-yl)methyl]piperidin-1-yl}phenyl)piperidine-2,6-dione

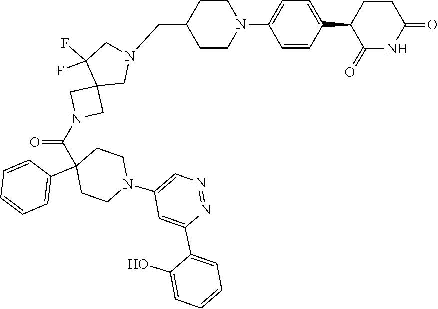 [SMILES: O=C1CC[C@H](c2ccc(N3CCC(CN4CC(F)(F)C5(C4)CN(C(=O)C4(c6ccccc6)CCN(c6cnnc(-c7ccccc7O)c6)CC4)C5)CC3)cc2)C(=O)N1]

Dissolved 2-[5-(4-{8,8-difluoro-2,6-diazaspiro[3.4]octane-2-carbonyl}-4-phenylpiperidin-1-yl)pyridazin-3-yl]phenol (60.00 mg, 0.1187 mmol), 1-{4-[(3R)-2,6-dioxopiperidin-3-yl]phenyl}piperidine-4-carbaldehyde (39.21 mg, 0.1305 mmol), and N,N-diisopropylethylamine (103.64 μL, 76.69 mg, 0.5934 mmol) in DCE and stirred for 30 min at room temperature. Then added sodium triacetoxyborohydride (75.46 mg, 0.3560 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (30.2 mg, 36%). LCMS: C₄₅H₄₉F₂N₇O₄ requires: 789.4. found: m/z=790.3 [M+H]⁺.

### Example 53

### (3R)-3-(4-(4-((4-(1-(6-(2-hydroxyphenyl)pyridazin-4-yl)-4-phenylpiperidine-4-carbonyl)-7-(trifluoromethyl)-1,4-diazepan-1-yl)methyl)piperidin-1-yl)phenyl)piperidine-2,6-dione

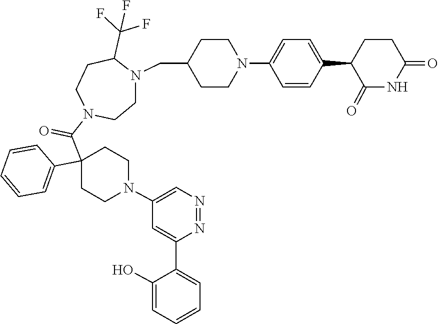 [SMILES: O=C1CC[C@H](c2ccc(N3CCC(CN4CCN(C(=O)C5(c6ccccc6)CCN(c6cnnc(-c7ccccc7O)c6)CC5)CCC4C(F)(F)F)CC3)cc2)C(=O)N1]

### Step 1: Synthesis of (1-(6-(2-hydroxyphenyl)pyridazin-4-yl)-4-phenylpiperidin-4-yl)(5-(trifluoromethyl)-1,4-diazepan-1-yl)methanone

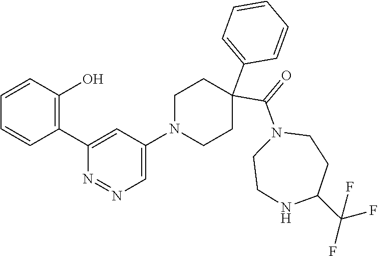 [SMILES: O=C(N1CCNC(C(F)(F)F)CC1)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added 5-(trifluoromethyl)-1,4-diazepane (24.14 mg, 0.1436 mmol) and stirred at room temperature until complete by LCMS. Directly injected on RP-FC and purified with a gradient of 5-80% MeCN in H₂O to yield the product (30 mg, 44%). LCMS: C₂₈H₃₀F₂N₅O₂ requires: 525.2. found: m/z=526.2 [M+H]⁺.

### Step 2: Synthesis of the Title Compound

Dissolved 2-(5-{4-phenyl-4-[5-(trifluoromethyl)-1,4-diazepane-1-carbonyl]piperidin-1-yl}pyridazin-3-yl)phenol (27.00 mg, 0.0514 mmol), 1-{4-[(3R)-2,6-dioxopiperidin-3-yl]phenyl}piperidine-4-carbaldehyde (16.97 mg, 0.0565 mmol), and N,N-diisopropylethylamine (44.86 μL, 33.20 mg, 0.2569 mmol) in DCE and stirred for 30 min at room temperature. Then added sodium triacetoxyborohydride (32.66 mg, 0.1541 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (6.5 mg, 16%). LCMS: C₄₅H₅₀F₃N₇O₄ requires: 809.4. found: m/z=810.3 [M+H]⁺.

### Example 54

### rac-(R)-3-(4-(4-((6-(1-(6-(2-hydroxyphenyl)pyridazin-4-yl)-4-(1-methyl-1H-pyrazol-3-yl)piperidine-4-carbonyl)-2,6-diazaspiro[3.3]heptan-2-yl)methyl)piperidin-1-yl)phenyl)piperidine-2,6-dione

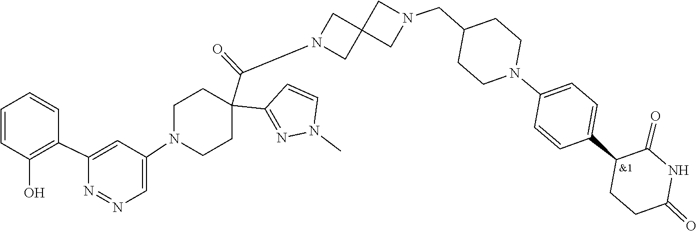 [SMILES: Cn1ccc(C2(C(=O)N3CC4(CN(CC5CCN(c6ccc([C@@H]7CCC(=O)NC7=O)cc6)CC5)C4)C3)CCN(c3cnnc(-c4ccccc4O)c3)CC2)n1]

### Step 1: Synthesis of 2-(1-methyl-1H-pyrazol-3-yl)acetonitrile

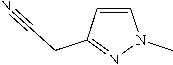

To a solution of 3-(chloromethyl)-1-methyl-1H-pyrazole (9.00 g, 68.9 mmol, 1.00 eq) in ACN (45.0 mL) and H₂O (9.00 mL) was added KCN (6.88 g, 106 mmol, 4.53 mL, 1.53 eq) at 25° C., then the mixture was heated to 50° C. and stirred for 12 hrs. The mixture was cooled down to 25° C. and adjusted the pH to 9 with saturated aqueous NaHCO₃ solution (200 mL). Then the mixture was extracted with EtOAc (3×100 mL). The organic layers were washed with brine (2×200 mL), dried over Na₂SO₄, filtered, and concentrated to give the product. The residue was purified by column chromatography (SiO₂, Petroleum ether/Ethyl acetate=I/O to 1/1, Petroleum ether/Ethyl acetate=1/1, R_{f}=0.40) to afford the title compound as a colorless oil (4.40 g, 35.7 mmol, 51.9% yield, 98.5% purity). LCMS: C₆H₇N₃ requires: 121.2. found: m/z=122.2 [M+H]⁺. ¹H NMR: (400 MHz, CDCl₃) δ 7.34 (d, J=2.0 Hz, 1H), 6.26 (d, J=2.0 Hz, 1H), 3.88 (s, 3H), 3.74 (s, 2H).

### Step 2: Synthesis of tert-butyl 4-cyano-4-(1-methyl-1H-pyrazol-3-yl)piperidine-1-carboxylate

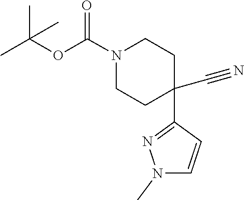 [SMILES: Cn1ccc(C2(C#N)CCN(C(=O)OC(C)(C)C)CC2)n1]

To a solution of 2-(1-methyl-1H-pyrazol-3-yl)acetonitrile (4.00 g, 33.0 mmol, 1.00 eq) in DMF (40.0 mL) was added NaH (2.91 g, 72.6 mmol, 60.0% purity, 2.20 eq) at 0° C., the mixture was stirred at 0° C. for 0.500 hr. Then tert-butyl bis(2-chloroethyl)carbamate (8.80 g, 36.3 mmol, 1.10 eq) was added, the mixture was heated to 60° C. and stirred for 2 hrs. The mixture was cooled down to 25° C., then poured into saturated aqueous NH₄Cl solution (200 mL), then extracted with EtOAc (3×100 mL). The combined organic layers were washed with brine (3×200 mL), dried over Na₂SO₄, filtered, and concentrated to give the product. The residue was purified by column chromatography (SiO₂, Petroleum ether/EtOAc=50/1 to 3/1) to yield the title compound as a colorless oil (6.50 g, 20.6 mmol, 62.6% yield, 92.4% purity). LCMS: C₁₅H₂₂N₄O₂ requires: 290.2, found: m/z=191.2 [M−Boc+H]⁺. ¹H NMR: (400 MHz, CDCl₃) δ 7.31 (d, J=2.4 Hz, 1H), 6.24 (d, J=2.4 Hz, 1H), 4.12 (br s, 2H), 3.87 (s, 3H), 3.20 (br s, 2H), 2.16 (br d, J=13.2 Hz, 2H), 1.99 (ddd, J=4.4, 12.0, 13.6 Hz, 2H), 1.45 (s, 9H).

### Step 3: Synthesis of 4-(1-methyl-1H-pyrazol-3-yl)piperidine-4-carbonitrile

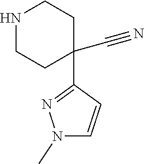

To a solution of tert-butyl 4-cyano-4-(1-methyl-1H-pyrazol-3-yl)piperidine-1-carboxylate (6.50 g, 20.6 mmol, 1.00 eq) in EtOAc (6.00 mL) was added HCl/EtOAc (4.00 M, 60.0 mL, 11.6 eq) at 25° C. for 12 hrs. The reaction mixture was filtered, and the filter cake was concentrated under vacuum to afford the title compound as a white solid (5.80 g, crude, HCl). ¹H NMR: (400 MHz, MeOD) δ 7.64 (d, J=2.0 Hz, 1H), 6.40 (d, J=2.0 Hz, 1H), 3.90 (s, 3H), 3.51 (td, J=4.4, 13.6 Hz, 2H), 3.42-3.32 (m, 2H), 2.55-2.35 (m, 4H).

### Step 4: Synthesis of 1-(6-chloropyridazin-4-yl)-4-(1-methyl-1H-pyrazol-3-yl)piperidine-4-carbonitrile

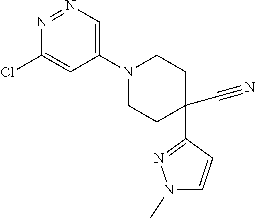 [SMILES: Cn1ccc(C2(C#N)CCN(c3cnnc(Cl)c3)CC2)n1]

To a solution of 4-(1-methyl-1H-pyrazol-3-yl)piperidine-4-carbonitrile (5.00 g, 21.9 mmol, 1.00 eq, HCl) and 3,5-dichloropyridazine (3.92 g, 26.3 mmol, 1.20 eq) in IPA (50.0 mL) was added DIEA (14.1 g, 109 mmol, 19.0 mL, 5.00 eq) at 25° C., then the mixture was stirred at 70° C. for 12 hrs. The reaction mixture was poured into H₂O (50.0 mL), then was extracted with EtOAc (3×50.0 mL). The combined organic layer was washed with brine (2×50.0 mL), dried over Na₂SO₄, filtered, and concentrated. The residue was purified by column chromatography (SiO₂, Petroleum ether/Ethyl acetate=10/1 to 3/1) to afford the title compound as a yellow solid (5.10 g, 16.5 mmol, 75.6% yield, 98.5% purity). LCMS: C₁₄H₁₅ClN₆ requires: 302.1. found: m/z=303.1 [M+H]⁺. ¹H NMR: (400 MHz, DMSO-d₆) δ 9.02 (d, J=2.8 Hz, 1H), 7.72 (d, J=2.4 Hz, 1H), 7.17 (d, J=2.8 Hz, 1H), 6.38 (d, J=2.4 Hz, 1H), 4.10 (br d, J=14.0 Hz, 2H), 3.83 (s, 3H), 3.34-3.21 (m, 2H), 2.24 (br d, J=14.0 Hz, 2H), 2.14-2.00 (m, 2H).

### Step 5: Synthesis of 1-(6-(2-hydroxyphenyl)pyridazin-4-yl)-4-(1-methyl-1H-pyrazol-3-yl)piperidine-4-carbonitrile

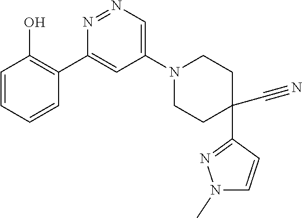 [SMILES: Cn1ccc(C2(C#N)CCN(c3cnnc(-c4ccccc4O)c3)CC2)n1]

To a solution of 1-(6-chloropyridazin-4-yl)-4-(1-methyl-1H-pyrazol-3-yl)piperidine-4-carbonitrile (2.50 g, 8.13 mmol, 1.00 eq), (2-hydroxyphenyl)boronic acid (2.24 g, 16.2 mmol, 2.00 eq), K₂CO₃ (3.37 g, 24.4 mmol, 3.00 eq) in dioxane (25.0 mL) and H₂O (5.00 mL) was added Pd(dppf)Cl₂ (1.19 g, 1.63 mmol, 0.200 eq) at 25° C., the mixture was stirred at 110° C. for 12 hrs. The reaction mixture was poured into H₂O (100 mL), then was extracted with EtOAc (3×100 mL). The combined organic layer was washed with brine (2×100 mL), dried over Na₂SO₄, filtered, and concentrated. The residue was purified by column chromatography (SiO₂, Petroleum ether/ethyl acetate=50/1 to 3/1) to afford the title compound as a yellow solid (1.47 g, 3.57 mmol, 43.9% yield, 87.6% purity) was obtained as a yellow solid. LCMS: C₂₀H₂₀N₆0 requires: 360.2. found: m/z=361.2 [M+H]⁺. ¹H NMR: (400 MHz, DMSO-d₆) δ 14.50 (br s, 1H), 9.00 (d, J=2.4 Hz, 1H), 8.12 (d, J=8.0 Hz, 1H), 7.72 (d, J=2.0 Hz, 1H), 7.63 (d, J=2.8 Hz, 1H), 7.38-7.27 (m, 1H), 7.00-6.90 (m, 2H), 6.39 (d, J=2.2 Hz, 1H), 4.28 (br d, J=14.0 Hz, 2H), 3.83 (s, 3H), 3.45-3.36 (m, 2H), 2.29 (br d, J=14.0 Hz, 2H), 2.19-2.04 (m, 2H).

### Step 6: Synthesis of 1-(6-(2-hydroxyphenyl)pyridazin-4-yl)-4-(1-methyl-1H-pyrazol-3-yl)piperidine-4-carboxylic acid

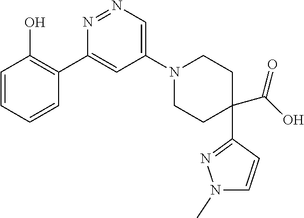 [SMILES: Cn1ccc(C2(C(=O)O)CCN(c3cnnc(-c4ccccc4O)c3)CC2)n1]

To a solution of 1-(6-(2-hydroxyphenyl)pyridazin-4-yl)-4-(1-methyl-1H-pyrazol-3-yl)piperidine-4-carbonitrile (1.47 g, 4.08 mmol, 1.00 eq) in H₂O (5.00 mL), MeOH (5.00 mL) and THF (5.00 mL) was added NaOH (1.63 g, 40.7 mmol, 10.0 eq) at 25° C., then mixture was stirred at 80° C. for 12 hrs. The reaction mixture was concentrated under vacuum. The crude product was purified by Prep-HPLC (column: Phenomenex luna C18 250*50 mm*10 um; mobile phase: [water (FA)-ACN]; gradient: 0%-30% B over 20 min), then concentrated by lyophilization to yield the title compound as a yellow solid (500 mg, 1.31 mmol, 23.6% yield, 99.6% purity). LCMS: C₂₀H₂₁N₅O₃ requires: 379.2. found: m/z=380.2 [M+H]⁺. ¹H NMR: (400 MHz, DMSO-d₆) δ 8.95 (d, J=2.8 Hz, 1H), 8.14 (s, 1H), 8.11 (dd, J=1.4, 8.4 Hz, 1H), 7.62 (d, J=2.4 Hz, 1H), 7.55 (d, J=2.8 Hz, 1H), 7.36-7.29 (m, 1H), 6.95-6.87 (m, 2H), 6.20 (d, J=2.4 Hz, 1H), 3.82-3.73 (m, 5H), 3.53-3.46 (m, 2H), 2.34-2.26 (m, 2H), 2.14-2.05 (m, 2H).

### Step 7: Synthesis of the Title Compound

1-[6-(2-hydroxyphenyl)pyridazin-4-yl]-4-(1-methylpyrazol-3-yl)piperidine-4-carboxylic acid (21.00 mg, 0.0553 mmol), PyBOP; hexafluoro-lambda5-phosphanuide (43.20 mg, 0.0830 mmol), and N,N-diisopropylethylamine (48.33 μL, 35.77 mg, 0.2767 mmol) were stirred in DMF for 1 hour at room temperature. rac-(3R)-3-[4-(4-{2,6-diazaspiro[3.3]heptan-2-ylmethyl}piperidin-1-yl)phenyl]piperidine-2,6-dione (27.52 mg, 0.0720 mmol) was then added and the reaction was stirred overnight. The solution was injected onto RP-FC and purified with a gradient of 0-70% MeCN in H₂O to furnish the title compound (8.2 mg, 20%). LCMS: C₄₂H₄₉N₉O₄ requires: 743.4, found: m/z=744.3 [M+H]⁺.

### Example 55

### (3R)-3-(4-{4-[(4-{1-[6-(2-hydroxyphenyl)pyridazin-4-yl]-4-phenylpiperidine-4-carbonyl}-5-methyl-1,4-diazepan-1-yl)methyl]piperidin-1-yl}phenyl)piperidine-2,6-dione

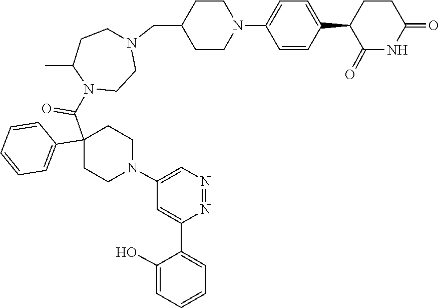 [SMILES: CC1CCN(CC2CCN(c3ccc([C@H]4CCC(=O)NC4=O)cc3)CC2)CCN1C(=O)C1(c2ccccc2)CCN(c2cnnc(-c3ccccc3O)c2)CC1]

### Step 1: Synthesis of 2-{5-[4-(4-benzyl-7-methyl-1,4-diazepane-1-carbonyl)-4-phenylpiperidin-1-yl]pyridazin-3-yl}phenol

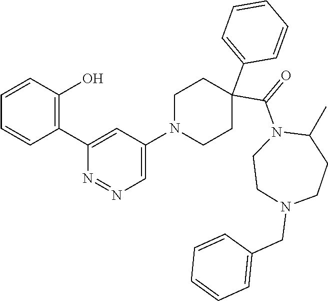 [SMILES: CC1CCN(Cc2ccccc2)CCN1C(=O)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added 1-benzyl-5-methyl-1,4-diazepane (34.67 mg, 0.1697 mmol) and stirred at room temperature until complete by LCMS. Directly injected onto RP-FC and purified using a gradient of 5-80% MeCN in water to yield the product (30 mg, 41%). LCMS: C₃₅H₃₉N₅O₂ requires: 561.3. found: m/z=562.3 [M+H]⁺.

### Step 2: Synthesis of 2-{5-[4-(7-methyl-1,4-diazepane-1-carbonyl)-4-phenylpiperidin-1-yl]pyridazin-3-yl}phenol

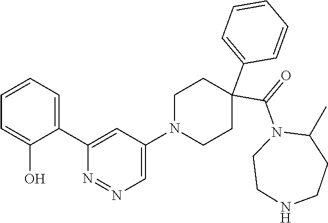 [SMILES: CC1CCNCCN1C(=O)C1(c2ccccc2)CCN(c2cnnc(-c3ccccc3O)c2)CC1]

2-{5-[4-(4-benzyl-7-methyl-1,4-diazepane-1-carbonyl)-4-phenylpiperidin-1-yl]pyridazin-3-yl}phenol (20.00 mg, 0.0340 mmol) and palladium on carbon (0.3 mg, 0.0003 mmol) were dissolved in dry MeOH and N₂ was bubbled through the solution using a balloon for 5 minutes. The balloon was then switched to H₂ and bubbled a further 5 minutes before being left to stir under H₂ atmosphere overnight. The palladium was removed by celite filtration to yield the product in quantitative yield, which was used in the next step without additional purification. LCMS: C₂₈H₃₃N₅O₂ requires: 471.3. found: m/z=472.3 [M+H]⁺.

### Step 3: Synthesis of the Title Compound

Dissolved 2-{5-[4-(7-methyl-1,4-diazepane-1-carbonyl)-4-phenylpiperidin-1-yl]pyridazin-3-yl}phenol (4.60 mg, 0.0098 mmol), 1-{4-[(3R)-2,6-dioxopiperidin-3-yl]phenyl}piperidine-4-carbaldehyde (3.22 mg, 0.0107 mmol), and N,N-diisopropylethylamine (8.52 μL, 6.30 mg, 0.0488 mmol) in DCE and stirred for 30 min at room temperature. Then added sodium triacetoxyborohydride (6.20 mg, 0.0293 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (2.8 mg, 38%). LCMS: C₄₅H₅₃N₇O₄ requires: 755.4. found: m/z=756.4 [M+H]⁺.

### Example 56

### N-[(3aRS,7aSR)-2-[(1-{4-[(3r)-2,6-dioxopiperidin-3-yl]phenyl}piperidin-4-yl)methyl]-octahydroisoindol-5-yl]-1-[6-(2-hydroxyphenyl)pyridazin-4-yl]-4-phenylpiperidine-4-carboxamide

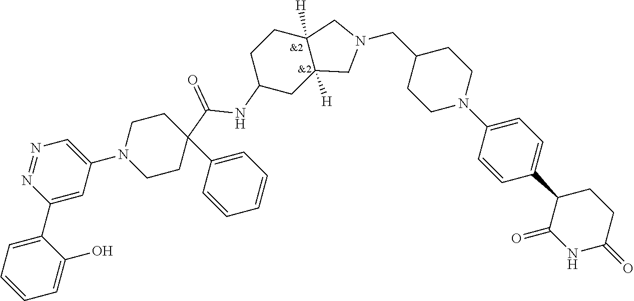 [SMILES: O=C1CC[C@H](c2ccc(N3CCC(CN4C[C@H]5CC(NC(=O)C6(c7ccccc7)CCN(c7cnnc(-c8ccccc8O)c7)CC6)CC[C@H]5C4)CC3)cc2)C(=O)N1]

### Step 1: Synthesis of N-[(3aS,7aR)-2-benzyl-octahydroisoindol-5-yl]-1-[6-(2-hydroxyphenyl)pyridazin-4-yl]-4-phenylpiperidine-4-carboxamide

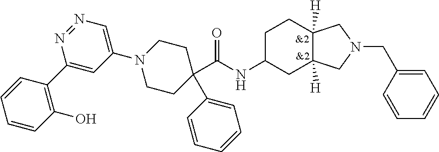 [SMILES: O=C(NC1CC[C@H]2CN(Cc3ccccc3)C[C@H]2C1)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (50.00 mg, 0.1332 mmol), [(dimethylamino)({[1,2,3]triazolo[4,5-b]pyridin-3-yloxy})methylidene]dimethylazanium; hexafluoro-lambda5-phosphanuide (126.60 mg, 0.3330 mmol), and N,N-diisopropylethylamine (116.31 μL, 86.07 mg, 0.6659 mmol) in DMF. Added (3aRS,7aRS&)-2-benzyl-octahydroisoindol-5-amine (39.88 mg, 0.1731 mmol) and stirred at room temperature until complete by LCMS. Directly injected onto RP-FC and purified using a gradient of 5-80% MeCN in water to yield the product (30 mg, 41%). LCMS: C₃₇H₄₁N₅O₂ requires: 587.3, found: m/z=588.3 [M+H]⁺.

### Step 2: Synthesis of rac-N-[(3aR,7aS)-octahydro-1H-isoindol-5-yl]-1-[6-(2-hydroxyphenyl)pyridazin-4-yl]-4-phenylpiperidine-4-carboxamide

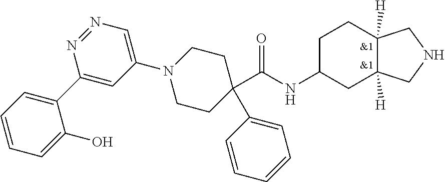 [SMILES: O=C(NC1CC[C@H]2CNC[C@H]2C1)C1(c2ccccc2)CCN(c2cnnc(-c3ccccc3O)c2)CC1]

N-[(3aS,7aR)-2-benzyl-octahydroisoindol-5-yl]-1-[6-(2-hydroxyphenyl)pyridazin-4-yl]-4-phenylpiperidine-4-carboxamide (20.00 mg, 0.0340 mmol) and palladium on carbon (0.3 mg, 0.0003 mmol) were dissolved in dry MeOH and N₂ was bubbled through the solution using a balloon for 5 minutes. The balloon was then switched to H₂ and bubbled a further 5 minutes before being left to stir under H₂ atmosphere overnight. The palladium was removed by celite filtration to yield the product in quantitative yield, which was used in the next step without additional purification. LCMS: C₃₀H₃₅N₅O₂ requires: 497.3. found: m/z=498.3 [M+H]⁺.

### Step 3: Synthesis of the Title Compound

Dissolved rac-N-[(3aR,7aS)-octahydro-1H-isoindol-5-yl]-1-[6-(2-hydroxyphenyl)pyridazin-4-yl]-4-phenylpiperidine-4-carboxamide (19.00 mg, 0.0382 mmol), 1-{4-[(3R)-2,6-dioxopiperidin-3-yl]phenyl}piperidine-4-carbaldehyde (12.61 mg, 0.0420 mmol), and N,N-diisopropylethylamine (33.34 μL, 24.67 mg, 0.1909 mmol) in DCE and stirred for 30 min at room temperature. Then added sodium triacetoxyborohydride (24.28 mg, 0.1145 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (16.4 mg, 52%). LCMS: C₄₇H₅₅N₇O₄ requires: 781.4. found: m/z=782.5 [M+H]⁺.

### Example 57

### (3R)-3-(4-{4-[(1-{1-[6-(2-hydroxyphenyl)pyridazin-4-yl]-4-phenylpiperidine-4-carbonyl}-hexahydro-2H-pyrrolo[3,4-b]pyridin-6-yl)methyl]piperidin-1-yl}phenyl)piperidine-2,6-dione

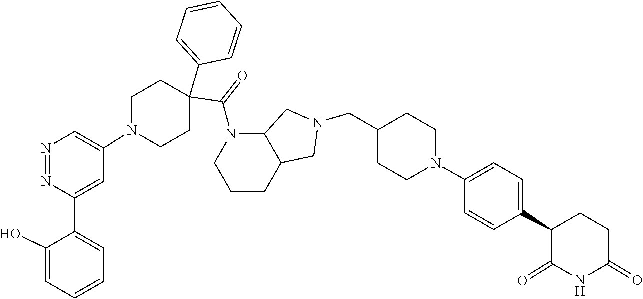 [SMILES: O=C1CC[C@H](c2ccc(N3CCC(CN4CC5CCCN(C(=O)C6(c7ccccc7)CCN(c7cnnc(-c8ccccc8O)c7)CC6)C5C4)CC3)cc2)C(=O)N1]

### Step 1: Synthesis of 2-[5-(4-{6-benzyl-hexahydro-2H-pyrrolo[3,4-b]pyridine-1-carbonyl}-4-phenylpiperidin-1-yl)pyridazin-3-yl]phenol

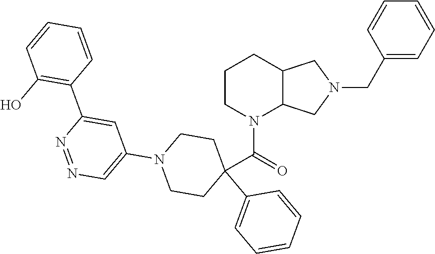 [SMILES: O=C(N1CCCC2CN(Cc3ccccc3)CC21)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added 6-benzyl-octahydropyrrolo[3,4-b]pyridine (38.12 mg, 0.1762 mmol) and stirred at room temperature until complete by LCMS. Directly injected onto RP-FC and purify using a gradient of 5-80% MeCN in water to yield the product (40 mg, 47%). LCMS: C₃₆H₃₉N₅O₂ requires: 573.3. found: m/z=574.3 [M+H]⁺.

### Step 2: Synthesis of 2-[5-(4-{octahydropyrrolo[3,4-b]pyridine-1-carbonyl}-4-phenylpiperidin-1-yl)pyridazin-3-yl]phenol

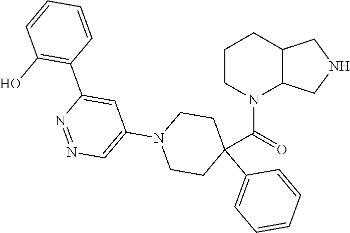 [SMILES: O=C(N1CCCC2CNCC21)C1(c2ccccc2)CCN(c2cnnc(-c3ccccc3O)c2)CC1]

2-[5-(4-{6-benzyl-hexahydro-2H-pyrrolo[3,4-b]pyridine-1-carbonyl}-4-phenylpiperidin-1-yl)pyridazin-3-yl]phenol (20.00 mg, 0.0340 mmol) and palladium on carbon (0.3 mg, 0.0003 mmol) were dissolved in dry MeOH and N₂ was bubbled through the solution using a balloon for 5 minutes. The balloon was then switched to H₂ and bubbled a further 5 minutes before being left to stir under H₂ atmosphere overnight. The palladium was removed by celite filtration to yield the product in quantitative yield, which was used in the next step without additional purification. LCMS: C₂₉H₃₃N₅O₂ requires: 483.3. found: m/z=484.3 [M+H]⁺.

### Step 3: Synthesis of the Title Compound

Dissolved 2-[5-(4-{octahydropyrrolo[3,4-b]pyridine-1-carbonyl}-4-phenylpiperidin-1-yl)pyridazin-3-yl]phenol (9.00 mg, 0.0186 mmol), 1-{4-[(3R)-2,6-dioxopiperidin-3-yl]phenyl}piperidine-4-carbaldehyde (6.15 mg, 0.0205 mmol), and N,N-diisopropylethylamine (16.25 μL, 12.03 mg, 0.0930 mmol) in DCE and stirred for 30 min at room temperature. Then added sodium triacetoxyborohydride (11.83 mg, 0.0558 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (5.1 mg, 34%). LCMS: C₄₆H₅₃N₇O₄ requires: 767.4. found: m/z=768.4 [M+H]⁺.

### Example 58

### (3R)-3-[4-(4-{[(3aRS,6aRS)-5-{1-[6-(2-hydroxyphenyl)pyridazin-4-yl]-4-phenylpiperidine-4-carbonyl}-octahydropyrrolo[3,4-b]pyrrol-1-yl]methyl}piperidin-1-yl)phenyl]piperidine-2,6-dione

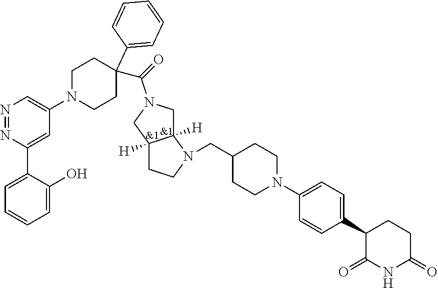 [SMILES: O=C1CC[C@H](c2ccc(N3CCC(CN4CC[C@H]5CN(C(=O)C6(c7ccccc7)CCN(c7cnnc(-c8ccccc8O)c7)CC6)C[C@H]54)CC3)cc2)C(=O)N1]

### Step 1: Synthesis of 2-(5-{4-[(3aS,6aS)-1-benzyl-hexahydropyrrolo[3,4-b]pyrrole-5-carbonyl]-4-phenylpiperidin-1-yl}pyridazin-3-yl)phenol

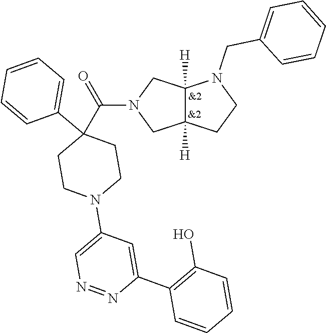 [SMILES: O=C(N1C[C@@H]2CCN(Cc3ccccc3)[C@@H]2C1)C1(c2ccccc2)CCN(c2cnnc(-c3ccccc3O)c2)CC1]

Stirred 1-[6-(2-hydroxyphenyl)pyridazin-4-yl]-4-phenylpiperidine-4-carboxylic acid (49.00 mg, 0.1305 mmol), [(dimethylamino)({[1,2,3]triazolo[4,5-b]pyridin-3-yloxy})methylidene]dimethylazanium; hexafluoro-lambda5-phosphanuide (124.07 mg, 0.3263 mmol), and N,N-diisopropylethylamine (113.98 μL, 84.35 mg, 0.6526 mmol) in DMF. Added (3aS,6aS)-1-benzyl-hexahydro-2H-pyrrolo[3,4-b]pyrrole (35.65 mg, 0.1762 mmol) and stirred at room temperature until complete by LCMS. Directly injected onto RP-FC and purified using a gradient of 5-80% MeCN in water to yield the product (80 mg, 94%). LCMS: C₃₆H₃₉N₅O₂ requires: 573.3. found: m/z=574.3 [M+H]⁺.

### Step 2: Synthesis ofrac-2-(5-{4-[(3aR,6aR)-octahydropyrrolo[3,4-b]pyrrole-5-carbonyl]-4-phenylpiperidin-1-yl}pyridazin-3-yl)phenol

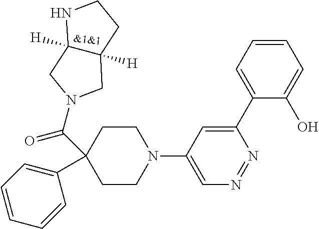 [SMILES: O=C(N1C[C@@H]2CCN[C@@H]2C1)C1(c2ccccc2)CCN(c2cnnc(-c3ccccc3O)c2)CC1]

2-(5-{4-[(3aS,6aS)-1-benzyl-hexahydropyrrolo[3,4-b]pyrrole-5-carbonyl]-4-phenylpiperidin-1-yl}pyridazin-3-yl)phenol (20.00 mg, 0.0340 mmol) and palladium on carbon (0.3 mg, 0.0003 mmol) were dissolved in dry MeOH and N₂ was bubbled through the solution using a balloon for 5 minutes. The balloon was then switched to H₂ and bubbled a further 5 minutes before being left to stir under H₂ atmosphere overnight. The palladium was removed by celite filtration to yield the product in quantitative yield, which was used in the next step without additional purification. LCMS: C₂₈H₃₁N₅O₂ requires: 469.2. found: m/z=470.3 [M+H]⁺.

### Step 3: Synthesis of the Title Compound

Dissolved rac-2-(5-{4-[(3aR,6aR)-hexahydro-1H-pyrrolo[3,4-b]pyrrole-5-carbonyl]-4-phenylpiperidin-1-yl}pyridazin-3-yl)phenol (17.30 mg, 0.0368 mmol), 1-{4-[(3R)-2,6-dioxopiperidin-3-yl]phenyl}piperidine-4-carbaldehyde (12.17 mg, 0.0405 mmol), and N,N-diisopropylethylamine (32.17 μL, 23.81 mg, 0.1842 mmol) in DCE and stirred for 30 min at room temperature. Then added sodium triacetoxyborohydride (23.42 mg, 0.1105 mmol) and stirred at room temperature overnight. Quenched the reaction by adding bicarbonate solution and extracted with EtOAc. Dried the organic layer with brine and then over Na₂SO₄. Concentrated and the solution was injected onto RP-FC and purified with a gradient of 0-70% MeCN in H₂O to furnish the title compound (13.9 mg, 47%). LCMS: C₄₅H₅₁N₇O₄ requires: 753.4. found: m/z=754.4 [M+H]⁺.

### Example 59

### N-{1-[(3RS)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methyl-4-[5-(propan-2-yl)-1,2-oxazol-3-yl]piperidine-4-carboxamide

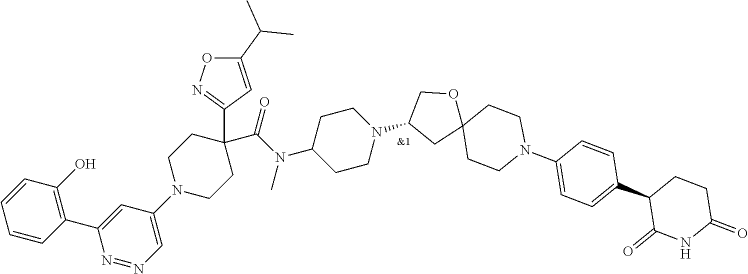 [SMILES: CC(C)c1cc(C2(C(=O)N(C)C3CCN([C@@H]4COC5(CCN(c6ccc([C@H]7CCC(=O)NC7=O)cc6)CC5)C4)CC3)CCN(c3cnnc(-c4ccccc4O)c3)CC2)no1]

Combined 1-[6-(2-hydroxyphenyl)pyridazin-4-yl]-4-(5-isopropyl-1,2-oxazol-3-yl)-N-methyl-N-(piperidin-4-yl)piperidine-4-carboxamide; trifluoroacetic acid (20.0 mg, 0.032 mmol) and (3R)-3-(4-{3-oxo-1-oxa-8-azaspiro[4.5]decan-8-yl}phenyl)piperidine-2,6-dione (12.18 mg, 0.036 mmol) in DMA (0.20 mL) and then added N,N-diisopropylethylamine (28.23 μL, 0.02 g, 0.162 mmol) and a solution of sodium triacetoxyborohydride (20.55 mg, 0.0970 mmol) in DMA (0.20 mL) and stirred overnight. Purified the solution directly by RP-HPLC to yield the title compound (8.6 mg, 28.6%).

LCMS: C₄₇H₅₈N₈O₆ requires: 830.4. found: m/z=831.4 [M+H]⁺.

### Example 60

### rac-N-{1-[2-(1-{4-[(3R)-2,6-dioxopiperidin-3-yl]-3-fluorophenyl}piperidin-4-yl)ethyl]piperidin-4-yl}-1-[6-(2-hydroxyphenyl)pyridazin-4-yl]-N-methyl-4-(3-methyl-1,2-oxazol-5-yl)piperidine-4-carboxamide

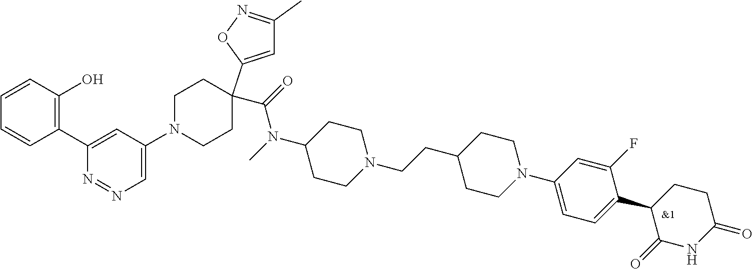 [SMILES: Cc1cc(C2(C(=O)N(C)C3CCN(CCC4CCN(c5ccc([C@H]6CCC(=O)NC6=O)c(F)c5)CC4)CC3)CCN(c3cnnc(-c4ccccc4O)c3)CC2)on1]

Combined 1-[6-(2-hydroxyphenyl)pyridazin-4-yl]-N-methyl-4-(3-methyl-1,2-oxazol-5-yl)-N-(piperidin-4-yl)piperidine-4-carboxamide; trifluoroacetic acid (20.0 mg, 0.034 mmol) and rac-2-(1-{4-[(3R)-2,6-dioxopiperidin-3-yl]-3-fluorophenyl}piperidin-4-yl)acetaldehyde (12.38 mg, 0.037 mmol) in DMA (0.20 mL) and then added N,N-diisopropylethylamine (29.57 μL, 0.02 g, 0.1693 mmol) and a solution of sodium triacetoxyborohydride (21.5 mg, 0.102 mmol) in DMA (0.20 mL) and stirred for 2 h. Added more aldehyde (7 mg) and STAB (15 mg) and stirred for another 30 min. Purified the solution directly by RP-HPLC to yield the title compound (7.4 mg, 24.7%). LCMS: C₄₄H₅₃FN₈O₅ requires: 792.4. found: m/z=793.3 [M+H]⁺.

### Example 61

### rac-N-{1-[2-(1-{1-[(3R)-2,6-dioxopiperidin-3-yl]-3-methyl-2-oxo-2,3-dihydro-1H-1,3-benzodiazol-4-yl}piperidin-4-yl)ethyl]piperidin-4-yl}-1-[6-(2-hydroxyphenyl)pyridazin-4-yl]-n-methyl-4-(5-methyl-1H-pyrazol-1-yl)piperidine-4-carboxamide

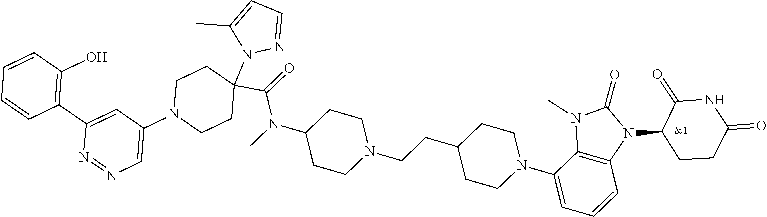 [SMILES: Cc1ccnn1C1(C(=O)N(C)C2CCN(CCC3CCN(c4cccc5c4n(C)c(=O)n5[C@@H]4CCC(=O)NC4=O)CC3)CC2)CCN(c2cnnc(-c3ccccc3O)c2)CC1]

Combined 1-[6-(2-hydroxyphenyl)pyridazin-4-yl]-N-methyl-4-(5-methylpyrazol-1-yl)-N-(piperidin-4-yl)piperidine-4-carboxamide; bis(trifluoroacetic acid) (18.01 mg, 0.0256 mmol) and rac-2-(1-{1-[(3R)-2,6-dioxopiperidin-3-yl]-3-methyl-2-oxo-1,3-benzodiazol-4-yl}piperidin-4-yl)acetaldehyde (10.82 mg, 0.0282 mmol)(synthesized as described in WO2024039901 A2 2024-02-22) in DMA (0.20 mL) and then added N,N-diisopropylethylamine (22.4 μL, 0.02 g, 0.1280 mmol) and a solution of sodium triacetoxyborohydride (16.3 mg, 0.077 mmol) in DMA (0.20 mL) and stirred for 2 h. Purified the solution directly by RP-HPLC to yield the title compound (4.5 mg, 18.9%). LCMS: C₄₆H₅₇N₁₁O₅ requires: 843.5. found: m/z=844.3 [M+H]⁺.

### Example 62

### rac-N-{1-[2-(1-{1-[(3R)-2,6-dioxopiperidin-3-yl]-3-methyl-2-oxo-2,3-dihydro-1H-1,3-benzodiazol-4-yl}piperidin-4-yl)ethyl]piperidin-4-yl}-1-[6-(2-hydroxyphenyl)pyridazin-4-yl]-N-methyl-4-(1-methyl-1H-pyrazol-5-yl)piperidine-4-carboxamide

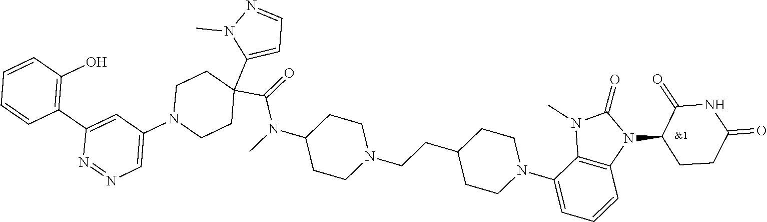 [SMILES: CN(C(=O)C1(c2ccnn2C)CCN(c2cnnc(-c3ccccc3O)c2)CC1)C1CCN(CCC2CCN(c3cccc4c3n(C)c(=O)n4[C@@H]3CCC(=O)NC3=O)CC2)CC1]

Combined 1-[6-(2-hydroxyphenyl)pyridazin-4-yl]-N-methyl-4-(2-methylpyrazol-3-yl)-N-(piperidin-4-yl)piperidine-4-carboxamide; bis(trifluoroacetic acid) (18.0 mg, 0.026 mmol) and rac-2-(1-{1-[(3R)-2,6-dioxopiperidin-3-yl]-3-methyl-2-oxo-1,3-benzodiazol-4-yl}piperidin-4-yl)acetaldehyde (10.8 mg, 0.028 mmol) in DMA (0.20 mL) and then added N,N-diisopropylethylamine (22.4 μL, 0.02 g, 0.13 mmol) and a solution of sodium triacetoxyborohydride (16.3 mg, 0.077 mmol) in DMA (0.20 mL) and stirred for 2 h. Purified the solution directly by RP-HPLC to yield the title compound (2.5 mg, 9.46%). LCMS: C₄₆H₅₇N₁₁O₅ requires: 843.5. found: m/z=844.3 [M+H]⁺.

### Example 63

### N-{1-[(3RS)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-n-methyl-4-(5-methyl-1H-pyrazol-1-yl)piperidine-4-carboxamide

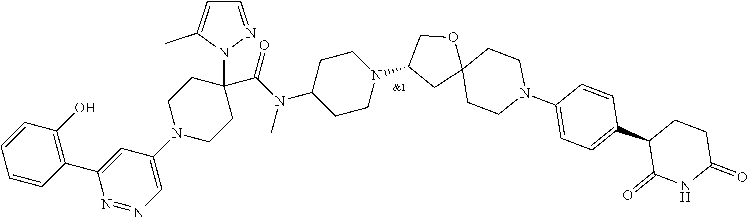 [SMILES: Cc1ccnn1C1(C(=O)N(C)C2CCN([C@@H]3COC4(CCN(c5ccc([C@H]6CCC(=O)NC6=O)cc5)CC4)C3)CC2)CCN(c2cnnc(-c3ccccc3O)c2)CC1]

Combined 1-[6-(2-hydroxyphenyl)pyridazin-4-yl]-N-methyl-4-(5-methylpyrazol-1-yl)-N-(piperidin-4-yl)piperidine-4-carboxamide; bis(trifluoroacetic acid) (18.0 mg, 0.026 mmol) and (3R)-3-(4-{3-oxo-1-oxa-8-azaspiro[4.5]decan-8-yl}phenyl)piperidine-2,6-dione (9.63 mg, 0.028 mmol) in DMA (0.20 mL) and then added N,N-diisopropylethylamine (22.3 μL, 0.02 g, 0.128 mmol) and a solution of sodium triacetoxyborohydride (16.27 mg, 0.077 mmol) in DMA (0.20 mL) and stirred overnight. Purified the solution directly by RP-HPLC to yield the title compound (2.2 mg, 9.8%). LCMS: C₄₅H₅₅N₉O₅ requires: 801.4. found: m/z=802.4 [M+H]⁺.

### Example 64

### N-{1-[(3RS)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-n-methyl-4-(1-methyl-1H-pyrazol-4-yl)piperidine-4-carboxamide

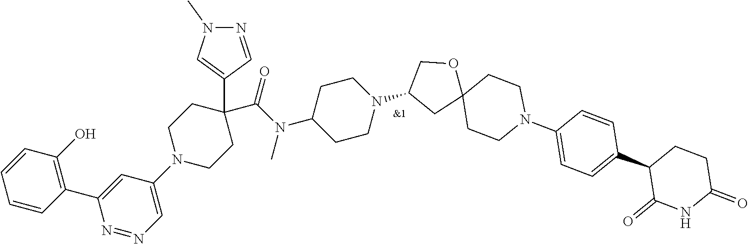 [SMILES: CN(C(=O)C1(c2cnn(C)c2)CCN(c2cnnc(-c3ccccc3O)c2)CC1)C1CCN([C@@H]2COC3(CCN(c4ccc([C@H]5CCC(=O)NC5=O)cc4)CC3)C2)CC1]

Combined 1-[6-(2-hydroxyphenyl)pyridazin-4-yl]-N-methyl-4-(1-methylpyrazol-4-yl)-N-(piperidin-4-yl)piperidine-4-carboxamide; trifluoroacetic acid (15.0 mg, 0.026 mmol) and (3R)-3-(4-{3-oxo-1-oxa-8-azaspiro[4.5]decan-8-yl}phenyl)piperidine-2,6-dione (9.59 mg, 0.0280 mmol) in DMA (0.20 mL) and then added N,N-diisopropylethylamine (22.2 μL, 0.02 g, 0.127 mmol) and a solution of sodium triacetoxyborohydride (16.2 mg, 0.076 mmol) in DMA (0.20 mL) and stirred overnight. Purified the solution directly by RP-HPLC the title compound (0.0052 g, 23.24%). LCMS: C₄₅H₅₅N₉O₅ requires: 801.4. found: m/z=802.3 [M+H]⁺.

### Example 65

### rac-N-{1-[2-(1-{4-[(3R)-2,6-dioxopiperidin-3-yl]-3-fluorophenyl}piperidin-4-yl)ethyl]piperidin-4-yl}-1-[6-(2-hydroxyphenyl)pyridazin-4-yl]-n-methyl-4-(5-methyl-1H-pyrazol-1-yl)piperidine-4-carboxamide

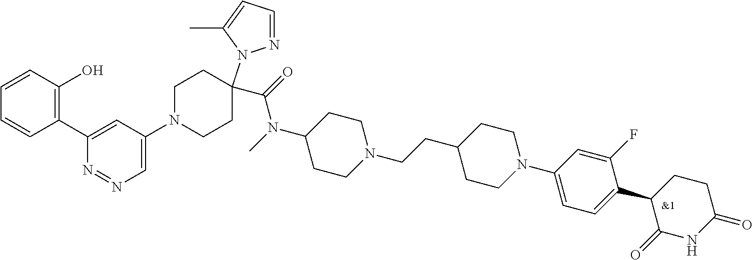 [SMILES: Cc1ccnn1C1(C(=O)N(C)C2CCN(CCC3CCN(c4ccc([C@H]5CCC(=O)NC5=O)c(F)c4)CC3)CC2)CCN(c2cnnc(-c3ccccc3O)c2)CC1]

Combined 1-[6-(2-hydroxyphenyl)pyridazin-4-yl]-N-methyl-4-(5-methylpyrazol-1-yl)-N-(piperidin-4-yl)piperidine-4-carboxamide; bis(trifluoroacetic acid) (18.0 mg, 0.026 mmol) and rac-2-(1-{4-[(3R)-2,6-dioxopiperidin-3-yl]-3-fluorophenyl}piperidin-4-yl)acetaldehyde (9.35 mg, 0.0281 mmol) in DMA (0.20 mL) and then added N,N-diisopropylethylamine (22.3 μL, 0.02 g, 0.128 mmol) and a solution of sodium triacetoxyborohydride (16.3 mg, 0.078 mmol) in DMA (0.20 mL) and stirred for 2 h. Added more aldehyde (7 mg) and STAB (15 mg) and stirred for another 30 min.

Purified the solution directly by RP-HPLC to yield the title compound (4.5 mg, 19.94%). LCMS: C₄₄H₅₄FN₉O₄ requires: 791.4. found: m/z=792.3 [M+H]⁺.

### Example 66

### rac-3-(2-fluoro-4-(4-(2-(6-(1-(6-(2-hydroxyphenyl)pyridazin-4-yl)-4-((1-methyl-1H-pyrazol-3-yl)oxy)piperidine-4-carbonyl)-2,6-diazaspiro[3.3]heptan-2-yl)ethyl)piperidin-1-yl)phenyl)piperidine-2,6-dione

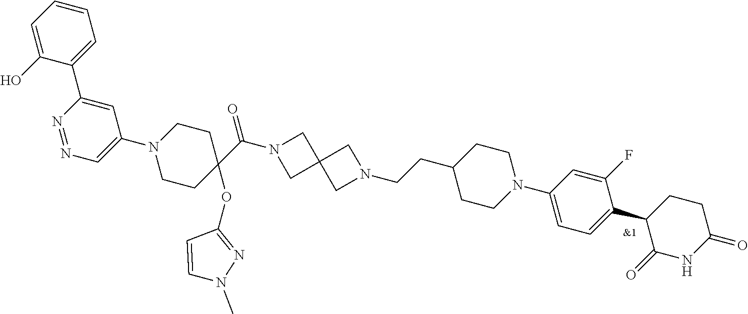 [SMILES: Cn1ccc(OC2(C(=O)N3CC4(CN(CCC5CCN(c6ccc([C@H]7CCC(=O)NC7=O)c(F)c6)CC5)C4)C3)CCN(c3cnnc(-c4ccccc4O)c3)CC2)n1]

### Step 1: Synthesis of (1-(6-(2-hydroxyphenyl)pyridazin-4-yl)-4-((1-methyl-1H-pyrazol-3-yl)oxy)piperidin-4-yl)(2,6-diazaspiro[3.3]heptan-2-yl)methanone

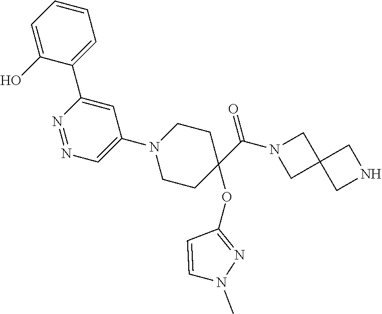 [SMILES: Cn1ccc(OC2(C(=O)N3CC4(CNC4)C3)CCN(c3cnnc(-c4ccccc4O)c3)CC2)n1]

1-(6-(2-hydroxyphenyl)pyridazin-4-yl)-4-((1-methyl-1H-pyrazol-3-yl)oxy)piperidine-4-carboxylic acid (Intermediate 21) (55.6 mg, 0.141 mmol) and tert-butyl 2,6-diazaspiro[3.3]heptane-2-carboxylate (27.9 mg, 0.141 mmol) were dissolved in 1.0 mL DMA. N,N-diisopropylethylamine (0.07 mL, 54.6 mg, 0.422 mmol) was then added followed by addition of [(dimethylamino)({[1,2,3]triazolo[4,5-b]pyridin-3-yloxy})methylidene]dimethylazanium; hexafluoro-lambda5-phosphanuide (80.2 mg, 0.21 mmol in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂₅H₂₉N₇O₃ requires 475.2. found: m/z=476.3 [M+H]⁺.

### Step 2: Synthesis of the Title Compound

(1-(6-(2-hydroxyphenyl)pyridazin-4-yl)-4-((1-methyl-1H-pyrazol-3-yl)oxy)piperidin-4-yl)(2,6-diazaspiro[3.3]heptan-2-yl)methanone (10.5 mg, 0.022 mmol) and rac-2-(1-(4-(2,6-dioxopiperidin-3-yl)-3-fluorophenyl)piperidin-4-yl)acetaldehyde (7.31 mg, 0.022 mmol) were dissolved in 0.5 mL DMA. N,N-diisopropylethylamine (14.3 mg, 0.110 mmol) followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H₂O to yield the title compound (3.0 mg, 17.2%). LCMS: C₄₃H₅₀FN₉O₆ requires 791.3. found: m/z=792.3 [M+H]⁺.

### Example 67

### rac-3-(4-(4-(2-(6-(1-(6-(2-hydroxyphenyl)pyridazin-4-yl)-4-((1-methyl-1H-pyrazol-3-yl)oxy)piperidine-4-carbonyl)-2,6-diazaspiro[3.3]heptan-2-yl)ethyl)piperidin-1-yl)-3-methyl-2-oxo-2,3-dihydro-1h-benzo[d]imidazol-1-yl)piperidine-2,6-dione

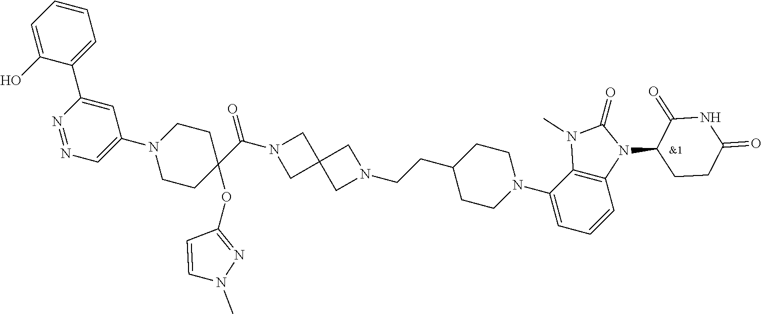 [SMILES: Cn1ccc(OC2(C(=O)N3CC4(CN(CCC5CCN(c6cccc7c6n(C)c(=O)n7[C@@H]6CCC(=O)NC6=O)CC5)C4)C3)CCN(c3cnnc(-c4ccccc4O)c3)CC2)n1]

### Step 1: Synthesis of the Title Compound

(1-(6-(2-hydroxyphenyl)pyridazin-4-yl)-4-((1-methyl-1H-pyrazol-3-yl)oxy)piperidin-4-yl)(2,6-diazaspiro[3.3]heptan-2-yl)methanone (10.5 mg, 0.022 mmol) and rac-2-(1-(1-(2,6-dioxopiperidin-3-yl)-3-methyl-2-oxo-2,3-dihydro-1H-benzo[d]imidazol-4-yl)piperidin-4-yl)acetaldehyde (8.45, 0.022 mmol) were dissolved in 0.5 mL DMA. N,N-diisopropylethylamine (14.3 mg, 0.110 mmol) followed by addition of sodium triacetoxyborohydride (23.3 mg, 0.11 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (7.2 mg, 38.8%). LCMS: C₄₅H₅₃N₁₁O₆ requires 843.4, found: m/z=844.4 [M+H]⁺.

### Example 68

### rac-N-(1-(2-(1-(1-(2,6-dioxopiperidin-3-yl)-3-methyl-2-oxo-2,3-dihydro-1H-benzo[d]imidazol-4-yl)piperidin-4-yl)ethyl)piperidin-4-yl)-1-(6-(2-hydroxyphenyl)pyridazin-4-yl)-4-methoxy-n-methylpiperidine-4-carboxamide

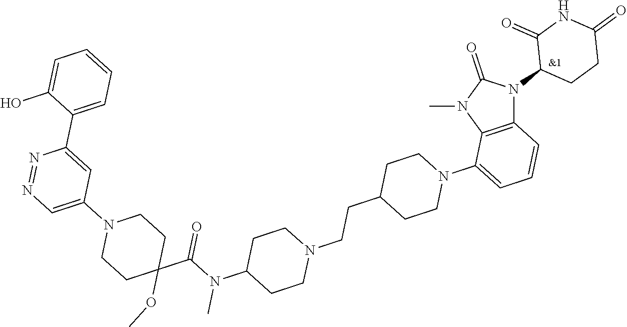 [SMILES: COC1(C(=O)N(C)C2CCN(CCC3CCN(c4cccc5c4n(C)c(=O)n5[C@@H]4CCC(=O)NC4=O)CC3)CC2)CCN(c2cnnc(-c3ccccc3O)c2)CC1]

### Step 1: Synthesis of (1-(6-(2-hydroxyphenyl)pyridazin-4-yl)-4-methoxy-N-methyl-N-(piperidin-4-yl)piperidine-4-carboxamide

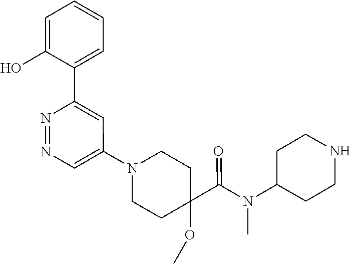 [SMILES: COC1(C(=O)N(C)C2CCNCC2)CCN(c2cnnc(-c3ccccc3O)c2)CC1]

1-(6-(2-hydroxyphenyl)pyridazin-4-yl)-4-methoxypiperidine-4-carboxylic acid (46.3 mg, 0.141 mmol) and tert-butyl 4-(methylamino)piperidine-1-carboxylate (0.03 mL, 30.2 mg, 0.141 mmol) were dissolved in 1.0 mL DMA. N,N-diisopropylethylamine (0.07 mL, 54.6 mg, 0.422 mmol) followed by addition of [(dimethylamino)({[1,2,3]triazolo[4,5-b]pyridin-3-yloxy})methylidene]dimethylazanium; hexafluoro-lambda5-phosphanuide (80.2 mg, 0.211 mmol in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂₃H₃₁N₅O₃ requires 425.2. found: m/z=426.2 [M+H]⁺.

### Step 2: Synthesis of the Title Compound

1-(6-(2-hydroxyphenyl)pyridazin-4-yl)-4-methoxy-N-methyl-N-(piperidin-4-yl)piperidine-4-carboxamide (9.36, 0.022 mmol) and rac-2-(1-(1-(2,6-dioxopiperidin-3-yl)-3-methyl-2-oxo-2,3-dihydro-1H-benzo[d]imidazol-4-yl)piperidin-4-yl)acetaldehyde (8.45, 0.022 mmol) were dissolved in 0.5 mL DMA. N,N-diisopropylethylamine (14.3 mg, 0.110 mmol) followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (5.0 mg, 28.6%). LCMS: C₄₃H₅₅N₉O₆ requires 793.4. found: m/z=794.4 [M+H]⁺.

### Example 69

### rac-N-(1-(2-(1-(5-(2,6-dioxopiperidin-3-yl)pyridin-2-yl)piperidin-4-yl)ethyl)piperidin-4-yl)-1-(6-(2-hydroxyphenyl)pyridazin-4-yl)-N-methyl-4-phenylpiperidine-4-carboxamide

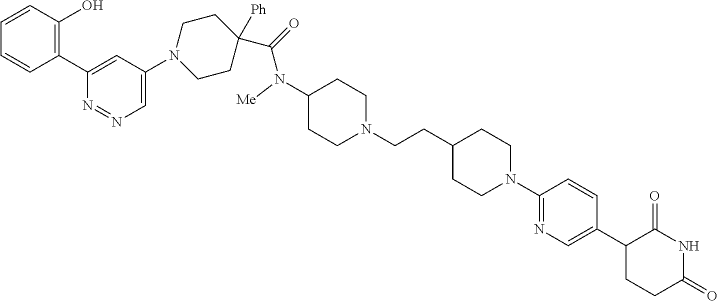 [SMILES: CN(C(=O)C1(c2ccccc2)CCN(c2cnnc(-c3ccccc3O)c2)CC1)C1CCN(CCC2CCN(c3ccc(C4CCC(=O)NC4=O)cn3)CC2)CC1]

### Step 1: Synthesis of 1-(6-(2-hydroxyphenyl)pyridazin-4-yl)-N-methyl-4-phenyl-N-(piperidin-4-yl)piperidine-4-carboxamide

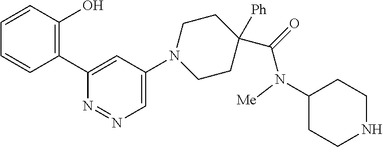 [SMILES: CN(C(=O)C1(c2ccccc2)CCN(c2cnnc(-c3ccccc3O)c2)CC1)C1CCNCC1]

This intermediate was synthesized according to Step 1 of Example 108 using tert-butyl 4-(methylamino)piperidine-1-carboxylate. LCMS: C₂₈H₃₃N₅O₂ requires 471.3. found: m/z=472.2 [M+H]⁺.

### Step 2: Synthesis of the Title Compound

The title compound was synthesized according to Step 3 of Example 145 using 1-(6-(2-hydroxyphenyl)pyridazin-4-yl)-N-methyl-4-phenyl-N-(piperidin-4-yl)piperidine-4-carboxamide and rac-(R)-2-(1-(5-(2,6-dioxopiperidin-3-yl)pyridin-2-yl)piperidin-4-yl)acetaldehyde (synthesized as described in WO2023023255 A1 2023-02-23). The solution was injected onto RP-FC and purified with a gradient of 5-95% MeCN in H₂O to furnish the title compound (6.1 mg, 13%). LCMS: C₄₅H₅₄N₈O₄ requires 770.4. found: m/z=771.4 [M+H]⁺.

### Example 70

### rac-(r)-4-cyclopropoxy-n-(1-(2-(1-(4-(2,6-dioxopiperidin-3-yl)-3-fluorophenyl)piperidin-4-yl)ethyl)piperidin-4-yl)-1-(6-(2-hydroxyphenyl)pyridazin-4-yl)-n-methylpiperidine-4-carboxamide

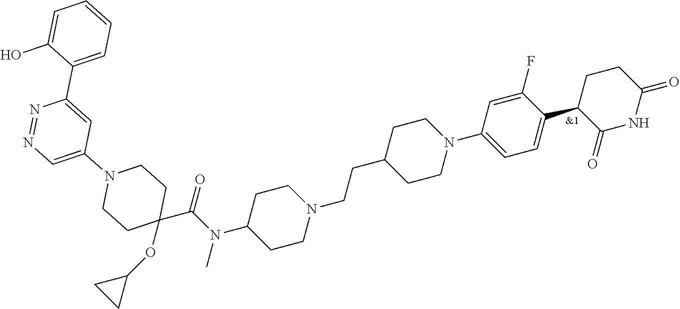 [SMILES: CN(C(=O)C1(OC2CC2)CCN(c2cnnc(-c3ccccc3O)c2)CC1)C1CCN(CCC2CCN(c3ccc([C@H]4CCC(=O)NC4=O)c(F)c3)CC2)CC1]

### Step 1: Synthesis of 4-cyclopropoxy-1-(6-(2-hydroxyphenyl)pyridazin-4-yl)-N-methyl-N-(piperidin-4-yl)piperidine-4-carboxamide

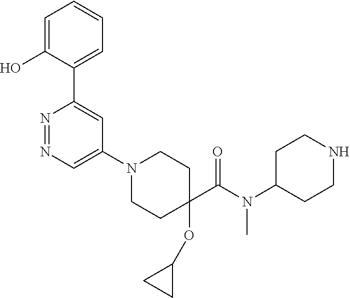 [SMILES: CN(C(=O)C1(OC2CC2)CCN(c2cnnc(-c3ccccc3O)c2)CC1)C1CCNCC1]

4-cyclopropoxy-1-[6-(2-hydroxyphenyl)pyridazin-4-yl]piperidine-4-carboxylic acid (Intermediate 22) (50.0 mg, 0.141 mmol) and tert-butyl 4-(methylamino)piperidine-1-carboxylate (0.03 mL, 30.12 mg, 0.141 mmol) were dissolved in 1.0 mL DMA. N,N-diisopropylethylamine (0.07 mL, 54.6 mg, 0.422 mmol) was added followed by addition of [(dimethylamino)({[1,2,3]triazolo[4,5-b]pyridin-3-yloxy})methylidene]dimethylazanium; hexafluoro-lambda5-phosphanuide (80.2 mg, 0.211 mmol) in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂₅H₃₃N₅O₃ requires 451.2. found: m/z=452.2 [M+H]⁺.

### Step 2: Synthesis of the Title Compound

4-cyclopropoxy-1-(6-(2-hydroxyphenyl)pyridazin-4-yl)-N-methyl-N-(piperidin-4-yl)piperidine-4-carboxamide (10.0 mg, 0.022 mmol) and rac-2-(1-(4-(2,6-dioxopiperidin-3-yl)-3-fluorophenyl)piperidin-4-yl)acetaldehyde (7.31 mg, 0.022 mmol) were dissolved in 0.5 mL DMA. N,N-diisopropylethylamine (14.3 mg, 0.110 mmol) followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (3.60 mg, 21.3%). LCMS: C₄₃H₅₄FN₇O₅ requires 767.4. found: m/z=768.4 [M+H]⁺.

### Example 71

### rac-N-(1-(2-(1-(4-(2,6-dioxopiperidin-3-yl)-3-fluorophenyl)piperidin-4-yl)ethyl)piperidin-4-yl)-1-(6-(2-hydroxyphenyl)pyridazin-4-yl)-4-methoxy-n-methylpiperidine-4-carboxamide

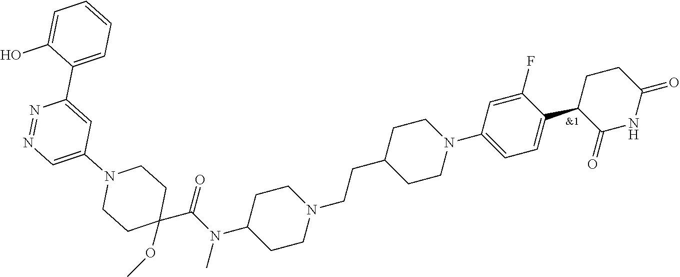 [SMILES: COC1(C(=O)N(C)C2CCN(CCC3CCN(c4ccc([C@H]5CCC(=O)NC5=O)c(F)c4)CC3)CC2)CCN(c2cnnc(-c3ccccc3O)c2)CC1]

### Step 1: Synthesis of the Title Compound

1-(6-(2-hydroxyphenyl)pyridazin-4-yl)-4-methoxy-N-methyl-N-(piperidin-4-yl)piperidine-4-carboxamide (9.36, 0.022 mmol) and rac-2-(1-(4-(2,6-dioxopiperidin-3-yl)-3-fluorophenyl)piperidin-4-yl)acetaldehyde (7.31 mg, 0.022 mmol) were dissolved in 0.5 mL DMA. N,N-diisopropylethylamine (14.3 mg, 0.110 mmol) followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (3.0 mg, 18.3%). LCMS: C₄₁H₅₂FN₇O₅ requires 741.4. found: m/z=742.4 [M+H]⁺.

### Example 72

### (1r,5s,6r)-3-(1-(1-((rs)-2,6-dioxopiperidin-3-yl)-3-methyl-2-oxo-2,3-dihydro-1h-benzo[d]imidazol-4-yl)piperidine-4-carbonyl)-n-((1-(6-(2-hydroxyphenyl)pyridazin-4-yl)-4-phenylpiperidin-4-yl)methyl)-3-azabicyclo[3.1.0]hexane-6-carboxamide

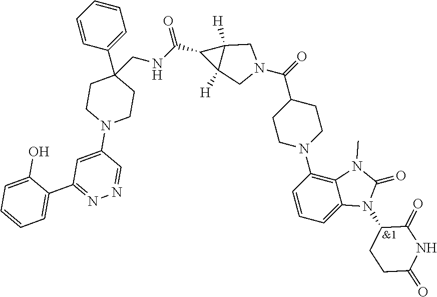 [SMILES: Cn1c(=O)n([C@H]2CCC(=O)NC2=O)c2cccc(N3CCC(C(=O)N4C[C@@H]5[C@H](C4)[C@H]5C(=O)NCC4(c5ccccc5)CCN(c5cnnc(-c6ccccc6O)c5)CC4)CC3)c21]

### Step 1: Synthesis of tert-butyl (1R,5S,6r)-6-(((1-(6-(2-hydroxyphenyl)240yridazine-4-yl)-4-phenylpiperidin-4-yl)methyl)carbamoyl)-3-azabicyclo[3.1.0]hexane-3-carboxylate

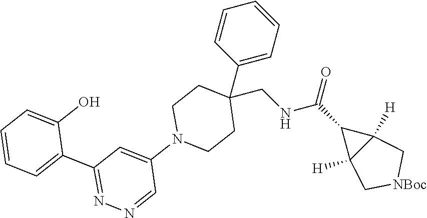 [SMILES: CC(C)(C)OC(=O)N1C[C@@H]2[C@H](C1)[C@H]2C(=O)NCC1(c2ccccc2)CCN(c2cnnc(-c3ccccc3O)c2)CC1]

This intermediate was synthesized as described for Example 104 Step 1, using (1R,5S,6r)-3-(tert-butoxycarbonyl)-3-azabicyclo[3.1.0]hexane-6-carboxylic acid in place of 1-(tert-butoxycarbonyl)-9-oxa-1-azaspiro[5.5]undecane-4-carboxylic acid. LCMS C₃₃H₃₉N₅O₄ requires 569.3, found m/z=570.3 [M+H]⁺.

### Step 2: Synthesis of (1R,5S,6r)-N-((1-(6-(2-hydroxyphenyl)240yridazine-4-yl)-4-phenylpiperidin-4-yl)methyl)-3-azabicyclo[3.1.0]hexane-6-carboxamide

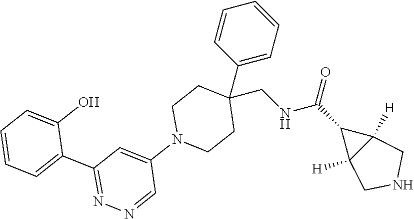 [SMILES: O=C(NCC1(c2ccccc2)CCN(c2cnnc(-c3ccccc3O)c2)CC1)[C@H]1[C@@H]2CNC[C@@H]21]

This intermediate was synthesized as described for the Example 104 Step 2, using tert-butyl (1R,5S,6r)-6-(((1-(6-(2-hydroxyphenyl)241yridazine-4-yl)-4-phenylpiperidin-4-yl)methyl)carbamoyl)-3-azabicyclo[3.1.0]hexane-3-carboxylate in place of tert-butyl 4-(((1-(6-(2-hydroxyphenyl)241yridazine-4-yl)-4-phenylpiperidin-4-yl)methyl)carbamoyl)-9-oxa-1-azaspiro[5.5]undecane-1-carboxylate. LCMS C₂₈H₃₁N₅O₂ requires 469.3, found m/z=470.3 [M+H]⁺.

### Step 3: Synthesis of the Title Compound

To a vial containing rac-®-1-(1-(2,6-dioxopiperidin-3-yl)-3-methyl-2-oxo-2,3-dihydro-1H-benzo[d]241yridazin-4-yl)piperidine-4-carboxylic acid (4.1 mgs, 0.011 mmol) (synthesized as described in WO2022235715 A1 2022-11-10) was added DMF (0.15 mL) followed by addition of DIPEA (1.35 μL, 0.011 mmol) and HATU (4.1 mgs, 0.011 mmol). See, also WO2022235715. This mixture was allowed to stir for 10 minutes at RT. In a separate vial, (1R,5S,6r)-N-((1-(6-(2-hydroxyphenyl)241yridazine-4-yl)-4-phenylpiperidin-4-yl)methyl)-3-azabicyclo[3.1.0]hexane-6-carboxamide (5.0 mgs, 0.011 mmol, Example 9 Step 2) was suspended in DMF (0.15 mL) and to the suspension was added DIPEA (1.35 μL, 0.011 mmol) and mixed until homogeneous. Once dissolved, the amine containing solution was added to the acid-containing solution in one portion and the reaction mixture was allowed to stir until completion as judged by LCMS. Once complete, the reaction mixture was directly injected onto RP-FC and purified with a gradient of 5-95% MeCN in H₂O to furnish the title compound (4.9 mgs, 55% yield over three steps). LCMS C₄₇H₅₁N₉O₆ requires 837.4, found m/z=838.4 [M+H]⁺.

### Example 73

### (1R,5S,6S)-3-[2-(4-{4-[(3RS)-2,6-dioxopiperidin-3-yl]phenyl}piperidin-1-yl)acetyl]-N-({1-[6-(2-hydroxyphenyl)pyridazin-4-yl]-4-phenylpiperidin-4-yl}methyl)-3-azabicyclo[3.1.0]hexane-6-carboxamide

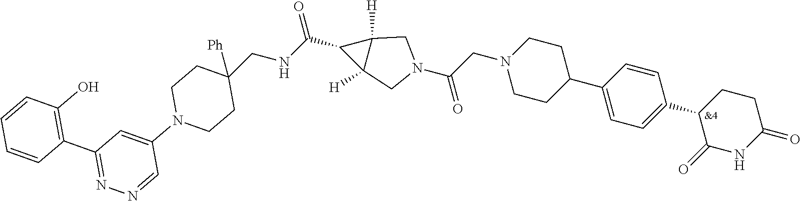 [SMILES: O=C1CC[C@@H](c2ccc(C3CCN(CC(=O)N4C[C@@H]5[C@H](C4)[C@H]5C(=O)NCC4(c5ccccc5)CCN(c5cnnc(-c6ccccc6O)c5)CC4)CC3)cc2)C(=O)N1]

LCMS: C₄₆H₅₁N₇O₅ requires: 781.4. found: m/z=782.1 [M+H]⁺.

### Example 74

### N-cyclopropyl-N-{1-[2-(1-{4-[(3r)-2,6-dioxopiperidin-3-yl]phenyl}piperidin-4-yl)ethyl]piperidin-4-yl}-1-[6-(2-hydroxyphenyl)pyridazin-4-yl]-4-phenylpiperidine-4-carboxamide

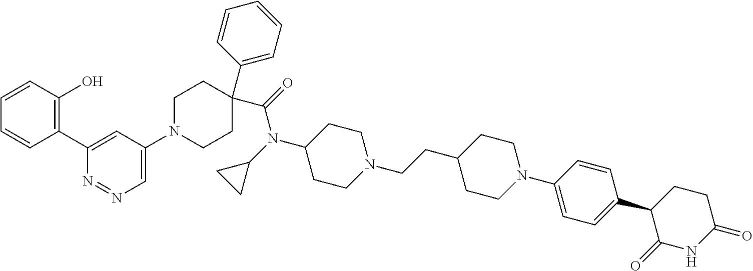 [SMILES: O=C1CC[C@H](c2ccc(N3CCC(CCN4CCC(N(C(=O)C5(c6ccccc6)CCN(c6cnnc(-c7ccccc7O)c6)CC5)C5CC5)CC4)CC3)cc2)C(=O)N1]

Combined N-cyclopropyl-1-[6-(2-hydroxyphenyl)pyridazin-4-yl]-4-phenyl-N-(piperidin-4-yl)piperidine-4-carboxamide (Intermediate 27); trifluoroacetic acid (7.0 mg, 0.011 mmol) and 2-(1-{4-[(3R)-2,6-dioxopiperidin-3-yl]phenyl}piperidin-4-yl)acetaldehyde (4.0 mg, 0.013 mmol) in DMA (0.20 mL) and then added N,N-diisopropylethylamine (6.0 μL, 0.034 mmol) and sodium triacetoxyborohydride (7.3 mg, 0.034 mmol) and stirred for 2 h. Purified the solution directly by RP-HPLC to yield the title compound (0.0056 g, 54.80%). LCMS: C₄₈H₅₇N₇O₄ requires: 795.4, found: m/z=796.4 [M+H]⁺.

### Example 75

### rac-N-{1-[2-(1-{4-[(3R)-2,6-dioxopiperidin-3-yl]-3-fluorophenyl}piperidin-4-yl)ethyl]piperidin-4-yl}-1-[6-(2-hydroxyphenyl)pyridazin-4-yl]-n-methyl-4-(pyridin-4-yl)piperidine-4-carboxamide

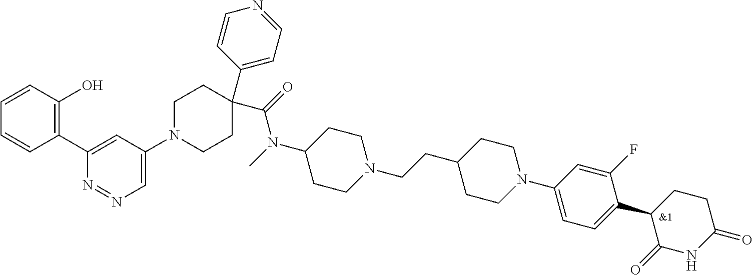 [SMILES: CN(C(=O)C1(c2ccncc2)CCN(c2cnnc(-c3ccccc3O)c2)CC1)C1CCN(CCC2CCN(c3ccc([C@H]4CCC(=O)NC4=O)c(F)c3)CC2)CC1]

Combined 1-[6-(2-hydroxyphenyl)pyridazin-4-yl]-N-methyl-N-(piperidin-4-yl)-4-(pyridin-4-yl)piperidine-4-carboxamide; trifluoroacetic acid (20.0 mg, 0.034 mmol) and rac-2-(1-{4-[(3R)-2,6-dioxopiperidin-3-yl]-3-fluorophenyl}piperidin-4-yl)acetaldehyde; trifluoroacetic acid (16.7 mg, 0.038 mmol) in DMA (0.20 mL) and then added N,N-diisopropylethylamine (17.9 μL, 0.01 g, 0.102 mmol) and sodium triacetoxyborohydride (21.7 mg, 0.10 mmol) and stirred for 2 h. Added additional portions of DIPEA and STAB and stirred overnight. Purified the solution directly by RP-HPLC to yield the title compound (6.7 mg, 23.2%). LCMS: C₄₅H₅₃FN₈O₄ requires: 788.4. found: m/z=789.3 [M+H]⁺.

### Example 76

### n-((2s,4r)-1-(2-(4-(4-((rs)-2,6-dioxopiperidin-3-yl)phenyl)piperidin-1-yl)acetyl)-2-methylpiperidin-4-yl)-1-(6-(2-hydroxyphenyl)pyridazin-4-yl)-4-phenylpiperidine-4-carboxamide

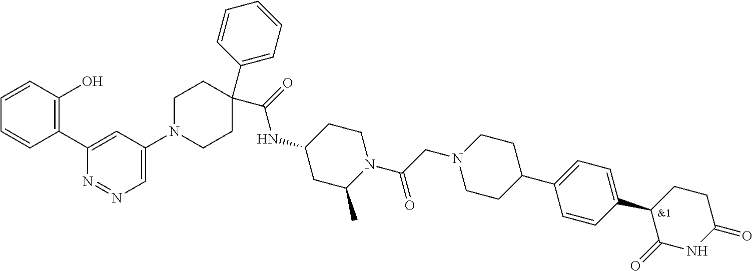 [SMILES: C[C@H]1C[C@H](NC(=O)C2(c3ccccc3)CCN(c3cnnc(-c4ccccc4O)c3)CC2)CCN1C(=O)CN1CCC(c2ccc([C@H]3CCC(=O)NC3=O)cc2)CC1]

### Step 1: Synthesis of 1-(6-(2-hydroxyphenyl)pyridazin-4-yl)-N-((2S,4R)-2-methylpiperidin-4-yl)-4-phenylpiperidine-4-carboxamide

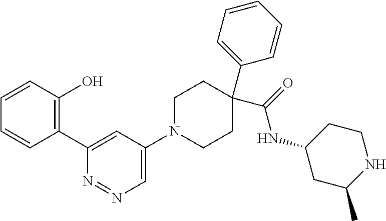 [SMILES: C[C@H]1C[C@H](NC(=O)C2(c3ccccc3)CCN(c3cnnc(-c4ccccc4O)c3)CC2)CCN1]

1-[6-(2-hydroxyphenyl)pyridazin-4-yl]-4-phenylpiperidine-4-carboxylic acid (75.00 mg, 0.1998 mmol) and tert-butyl (2S,4R)-4-amino-2-methylpiperidine-1-carboxylate (50.0 mg, 0.233 mmol) were dissolved in DMA (4.00 mL) and N,N-diisopropylethylamine (105.00 μL, 0.08 g, 0.603 mmol) and then [(dimethylamino)({[1,2,3]triazolo[4,5-b]pyridin-3-yloxy})methylidene]dimethylazanium; hexafluoro-lambda5-phosphanuide (91.15 mg, 0.24 mmol) was added. The resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂₈H₃₃N₅O₂ requires 471.2. found: m/z=472.6 [M+H]⁺.

### Step 1: Synthesis of the Title Compound

1-(6-(2-hydroxyphenyl)pyridazin-4-yl)-N-((2S,4R)-2-methylpiperidin-4-yl)-4-phenylpiperidine-4-carboxamide (10.3 mg, 0.022 mmol) and rac-2-(4-(4-(2,6-dioxopiperidin-3-yl)phenyl)piperidin-1-yl)acetic acid (7.31 mg, 0.022 mmol) (synthesized as described in WO2022032026 A1 2022-02-10) were dissolved in 0.5 mL DMA. N,N-diisopropylethylamine (14.3 mg, 0.110 mmol) was added followed by addition of [(dimethylamino)({[1,2,3]triazolo[4,5-b]pyridin-3-yloxy})methylidene]dimethylazanium; hexafluoro-lambda5-phosphanuide (12.6 mg, 0.033 mmol) in one portion and the resulting solution was stirred at room temperature for 1 h. The reaction was directly injected onto RP-FC and purified with a gradient of 0-80% MeCN in H₂O to yield the title compound (5.1 mg, 29.6%). LCMS: C₄₆H₅₃N₇O₅ requires 783.4. found: m/z=784.4 [M+H]⁺.

### Example 77

### rac-N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N-methyl-4-{pyrazolo[1,5-a]pyridin-2-yl}piperidine-4-carboxamide

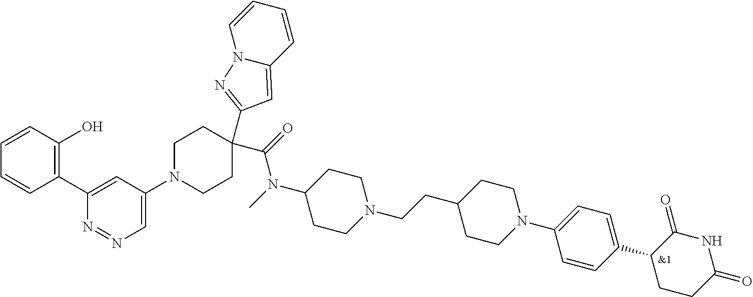 [SMILES: CN(C(=O)C1(c2cc3ccccn3n2)CCN(c2cnnc(-c3ccccc3O)c2)CC1)C1CCN(CCC2CCN(c3ccc([C@H]4CCC(=O)NC4=O)cc3)CC2)CC1]

This compound was synthesis according to step 2 of example 98 using 1-[6-(2-hydroxyphenyl)pyridazin-4-yl]-N-methyl-N-(piperidin-4-yl)-4-{pyrazolo[1,5-a]pyridin-2-yl}piperidine-4-carboxamide and 2-{1-[4-(2,6-dioxopiperidin-3-yl)phenyl]piperidin-4-yl}acetaldehyde. LCMS: C₄₇H₅₅N₉O₄ requires: 809.4. found: m/z=810.3 [M+H]⁺.

### Examples 78 and 78A

### rac-N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N-methyl-4-(5-methyl-1H-pyrazol-1-yl)piperidine-4-carboxamide

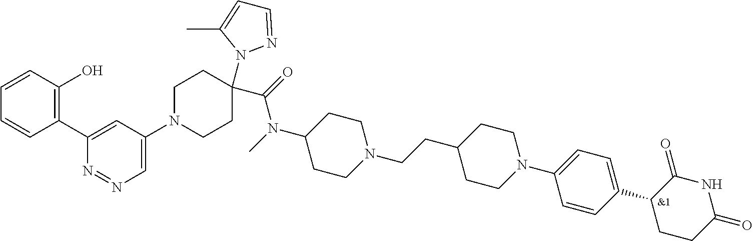 [SMILES: Cc1ccnn1C1(C(=O)N(C)C2CCN(CCC3CCN(c4ccc([C@H]5CCC(=O)NC5=O)cc4)CC3)CC2)CCN(c2cnnc(-c3ccccc3O)c2)CC1]

### N-{1-[2-(1-{4-[(3S)-2,6-dioxopiperidin-3-yl]phenyl}piperidin-4-yl)ethyl]piperidin-4-yl}-1-[6-(2-hydroxyphenyl)pyridazin-4-yl]-N-methyl-4-(5-methyl-1H-pyrazol-1-yl)piperidine-4-carboxamide

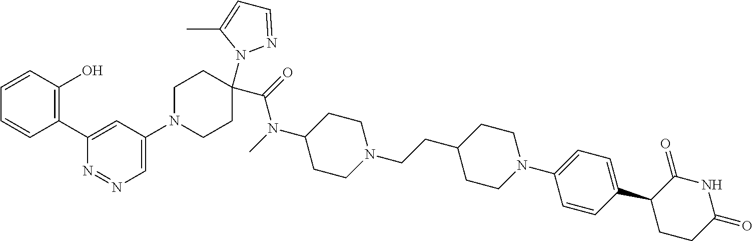 [SMILES: Cc1ccnn1C1(C(=O)N(C)C2CCN(CCC3CCN(c4ccc([C@@H]5CCC(=O)NC5=O)cc4)CC3)CC2)CCN(c2cnnc(-c3ccccc3O)c2)CC1]

This compound was synthesis according to step 2 of example 98 using 1-[6-(2-hydroxyphenyl)pyridazin-4-yl]-N-methyl-4-(5-methylpyrazol-1-yl)-N-(piperidin-4-yl)piperidine-4-carboxamide and 2-{1-[4-(2,6-dioxopiperidin-3-yl)phenyl]piperidin-4-yl}acetaldehyde. LCMS: C₄₄H₅₅N₉O₄ requires: 773.4. found: m/z=774.4 [M+H]⁺.

### Example 79

### rac-(3R)-3-(4-{4-[2-(6-{1-[6-(2-hydroxyphenyl)pyridazin-4-yl]-4-[5-(propan-2-yl)-1,2-oxazol-3-yl]piperidine-4-carbonyl}-2,6-diazaspiro[3.3]heptan-2-yl)ethyl]piperidin-1-yl}phenyl)piperidine-2,6-dione

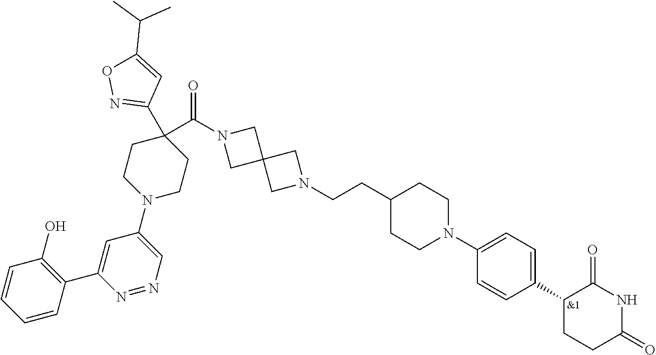 [SMILES: CC(C)c1cc(C2(C(=O)N3CC4(CN(CCC5CCN(c6ccc([C@H]7CCC(=O)NC7=O)cc6)CC5)C4)C3)CCN(c3cnnc(-c4ccccc4O)c3)CC2)no1]

LCMS: C₄₅H₅₄N₈O₅ requires: 786.4. found: m/z=787.4 [M+H]⁺.

### Example 80

### rac-(3R)-3-(4-{4-[2-(6-{1-[6-(2-hydroxyphenyl)pyridazin-4-yl]-4-{pyrazolo[1,5-a]pyridin-2-yl}piperidine-4-carbonyl}-2,6-diazaspiro[3.3]heptan-2-yl)ethyl]piperidin-1-yl}phenyl)piperidine-2,6-dione

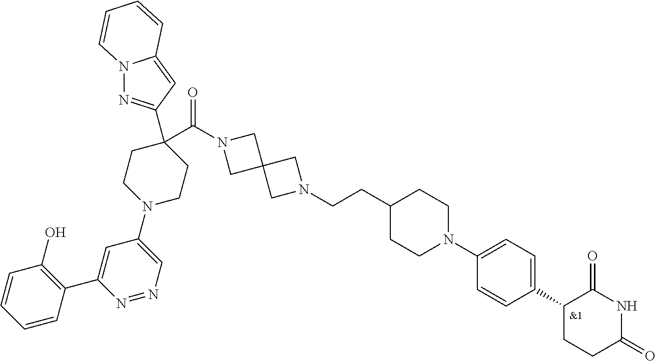 [SMILES: O=C1CC[C@H](c2ccc(N3CCC(CCN4CC5(C4)CN(C(=O)C4(c6cc7ccccn7n6)CCN(c6cnnc(-c7ccccc7O)c6)CC4)C5)CC3)cc2)C(=O)N1]

### Step 1: Synthesis of 2-[5-(4-{2,6-diazaspiro[3.3]heptane-2-carbonyl}-4-{pyrazolo[1,5-a]pyridin-2-yl}piperidin-1-yl)pyridazin-3-yl]phenol

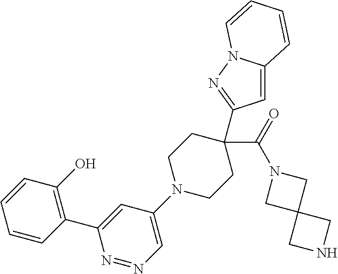 [SMILES: O=C(N1CC2(CNC2)C1)C1(c2cc3ccccn3n2)CCN(c2cnnc(-c3ccccc3O)c2)CC1]

This intermediate was synthesized according to step 1 of Example 98 using 1-[6-(2-hydroxyphenyl)pyridazin-4-yl]-4-{pyrazolo[1,5-a]pyridin-2-yl}piperidine-4-carboxylic acid (Intermediate 11) and tert-butyl 2,6-diazaspiro[3.3]heptane-2-carboxylate. LCMS: C₂₈H₂₉N₇O₂ requires: 495.2. found: m/z=496.4 [M+H]⁺.

### Step 2: Synthesis of the Title Compound

This compound was synthesis according to step 2 of example 98 using 2-[5-(4-{2,6-diazaspiro[3.3]heptane-2-carbonyl}-4-{pyrazolo[1,5-a]pyridin-2-yl}piperidin-1-yl)pyridazin-3-yl]phenol and 2-{1-[4-(2,6-dioxopiperidin-3-yl)phenyl]piperidin-4-yl}acetaldehyde. LCMS: C₄₆H₅₁N₉O₄ requires: 793.4. found: m/z=794.4 [M+H]⁺.

### Example 81

### rac-4-cyclopropoxy-n-(1-((1-(4-(2,6-dioxopiperidin-3-yl)phenyl)piperidin-4-yl)methyl)piperidin-4-yl)-1-(6-(2-hydroxyphenyl)pyridazin-4-yl)-n-methylpiperidine-4-carboxamide

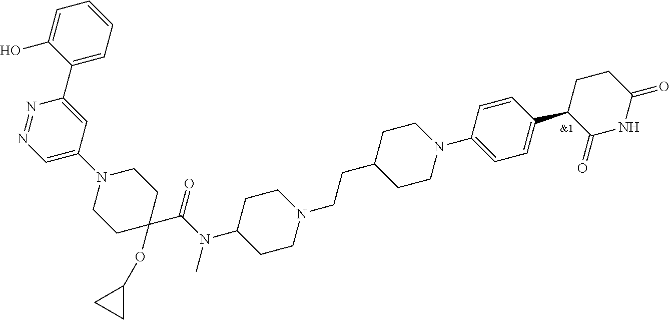 [SMILES: CN(C(=O)C1(OC2CC2)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of the Title Compound

4-cyclopropoxy-1-(6-(2-hydroxyphenyl)pyridazin-4-yl)-N-methyl-N-(piperidin-4-yl)piperidine-4-carboxamide (10.0 mg, 0.022 mmol) and rac-1-(4-(2,6-dioxopiperidin-3-yl)phenyl)piperidine-4-carbaldehyde (6.91 mg, 0.022 mmol) were dissolved in 0.5 mL DMA. N,N-diisopropylethylamine (14.3 mg, 0.110 mmol) was added followed by addition of sodium triacetoxyborohydride triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (5.8 mg, 35.2%). LCMS: C₄₃H₅₅N₇O₅ requires 749.4. found: m/z=750.4 [M+H]⁺.

### Example 82

### rac-3-(4-(4-(2-(6-(1-(6-(2-hydroxyphenyl)pyridazin-4-yl)-4-methoxypiperidine-4-carbonyl)-2,6-diazaspiro[3.3]heptan-2-yl)ethyl)piperidin-1-yl)phenyl)piperidine-2,6-dione

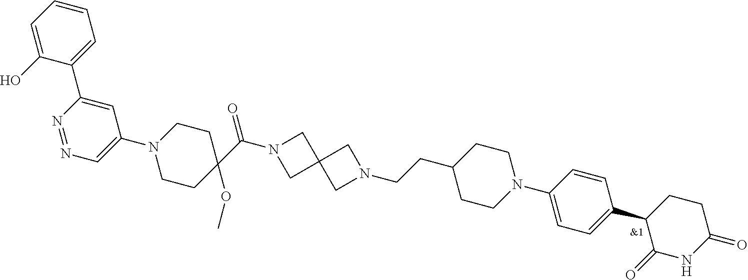 [SMILES: COC1(C(=O)N2CC3(CN(CCC4CCN(c5ccc([C@H]6CCC(=O)NC6=O)cc5)CC4)C3)C2)CCN(c2cnnc(-c3ccccc3O)c2)CC1]

### Step 1: Synthesis of (1-(6-(2-hydroxyphenyl)pyridazin-4-yl)-4-methoxypiperidin-4-yl)(2,6-diazaspiro[3.3]heptan-2-yl)methanone

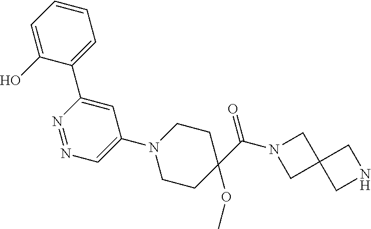 [SMILES: COC1(C(=O)N2CC3(CNC3)C2)CCN(c2cnnc(-c3ccccc3O)c2)CC1]

1-(6-(2-hydroxyphenyl)pyridazin-4-yl)-4-methoxypiperidine-4-carboxylic acid (46.3 mg, 0.141 mmol) and tert-butyl 2,6-diazaspiro[3.3]heptane-2-carboxylate (27.9 mg, 0.141 mmol) were dissolved in 1.0 mL DMA. N,N-diisopropylethylamine (0.07 mL, 54.6 mg, 0.422 mmol) was added followed by addition of [(dimethylamino)({[1,2,3]triazolo[4,5-b]pyridin-3-yloxy})methylidene]dimethylazanium; hexafluoro-lambda5-phosphanuide (80.24 mg, 0.2110 mmol in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂₂H₂₇N₅O₃ requires 409.2. found: m/z=410.4 [M+H]⁺.

### Step 2: Synthesis of the Title Compound

(1-(6-(2-hydroxyphenyl)pyridazin-4-yl)-4-methoxypiperidin-4-yl)(2,6-diazaspiro[3.3]heptan-2-yl)methanone (9.00 mg, 0.022 mmol) and rac-1-(4-(2,6-dioxopiperidin-3-yl)phenyl)piperidine-4-carbaldehyde (6.91 mg, 0.022 mmol) were dissolved in 0.5 mL DMA. N,N-diisopropylethylamine (14.3 mg, 0.110 mmol) followed by addition of sodium triacetoxyborohydride triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (5.6 mg, 35.9%). LCMS: C₄₀H₄₉N₇O₅ requires 707.4. found: m/z=708.3 [M+H]⁺.

### Example 83

### rac-(r)-1-(2-(1-(4-(2,6-dioxopiperidin-3-yl)phenyl)piperidin-4-yl)ethyl)-n-((1-(6-(2-hydroxyphenyl)pyridazin-4-yl)-4-phenylpiperidin-4-yl)methyl)-4-methoxypiperidine-4-carboxamide

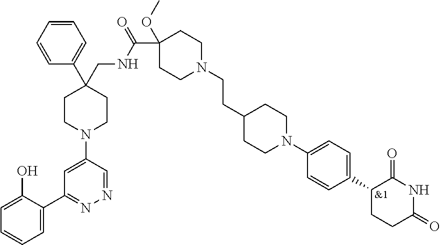 [SMILES: COC1(C(=O)NCC2(c3ccccc3)CCN(c3cnnc(-c4ccccc4O)c3)CC2)CCN(CCC2CCN(c3ccc([C@H]4CCC(=O)NC4=O)cc3)CC2)CC1]

### Step 1: Synthesis of tert-butyl 4-(((1-(6-(2-hydroxyphenyl)pyridazin-4-yl)-4-phenylpiperidin-4-yl)methyl)carbamoyl)-4-methoxypiperidine-1-carboxylate

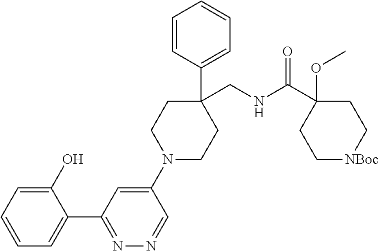 [SMILES: COC1(C(=O)NCC2(c3ccccc3)CCN(c3cnnc(-c4ccccc4O)c3)CC2)CCN(C(=O)OC(C)(C)C)CC1]

This intermediate was synthesized as described for Example 2 Step 104, using 1-(tert-butoxycarbonyl)-4-methoxypiperidine-4-carboxylic acid in place of 1-(tert-butoxycarbonyl)-9-oxa-1-azaspiro[5.5]undecane-4-carboxylic acid. LCMS C₃₄H₄₃N₅O₅ requires 601.3, found m/z=602.3 [M+H]⁺.

### Step 2: Synthesis of N-((1-(6-(2-hydroxyphenyl)pyridazin-4-yl)-4-phenylpiperidin-4-yl)methyl)-4-methoxypiperidine-4-carboxamide

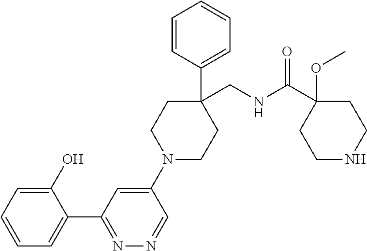 [SMILES: COC1(C(=O)NCC2(c3ccccc3)CCN(c3cnnc(-c4ccccc4O)c3)CC2)CCNCC1]

This intermediate was synthesized as described for Example 104 Step 2, using tert-butyl 4-(((1-(6-(2-hydroxyphenyl)pyridazin-4-yl)-4-phenylpiperidin-4-yl)methyl)carbamoyl)-4-methoxypiperidine-1-carboxylate in place of tert-butyl 4-(((1-(6-(2-hydroxyphenyl)pyridazin-4-yl)-4-phenylpiperidin-4-yl)methyl)carbamoyl)-9-oxa-1-azaspiro[5.5]undecane-1-carboxylate. LCMS C₂₉H₃₅N₅O₃ requires 501.3, found m/z 502.3 [M+H]⁺.

### Step 3: Synthesis of the Title Compound

The title compound was synthesized as described for Example 104 Step 3, using N-((1-(6-(2-hydroxyphenyl)pyridazin-4-yl)-4-phenylpiperidin-4-yl)methyl)-4-methoxypiperidine-4-carboxamide in place of in place of N-((1-(6-(2-hydroxyphenyl)pyridazin-4-yl)-4-phenylpiperidin-4-yl)methyl)-9-oxa-1-azaspiro[5.5]undecane-4-carboxamide (22.3 mg, 56% yield over three steps). LCMS C₄₇H₅₇N₇O₅ requires 799.4, found m/z=800.4 [M+H]⁺.

### Example 84

### rac-2-(2-(1-(4-((r)-2,6-dioxopiperidin-3-yl)phenyl)piperidin-4-yl)ethyl)-n-((1-(6-(2-hydroxyphenyl)pyridazin-4-yl)-4-phenylpiperidin-4-yl)methyl)-2-azabicyclo[4.1.0]heptane-5-carboxamide

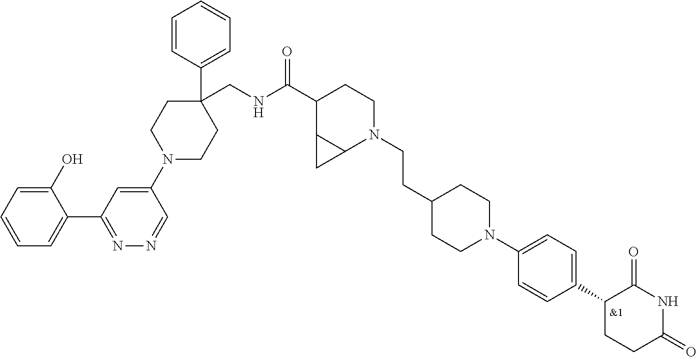 [SMILES: O=C1CC[C@H](c2ccc(N3CCC(CCN4CCC(C(=O)NCC5(c6ccccc6)CCN(c6cnnc(-c7ccccc7O)c6)CC5)C5CC54)CC3)cc2)C(=O)N1]

### Step 1: Synthesis of tert-butyl 5-(((1-(6-(2-hydroxyphenyl)pyridazin-4-yl)-4-phenylpiperidin-4-yl)methyl)carbamoyl)-2-azabicyclo[4.1.0]heptane-2-carboxylate

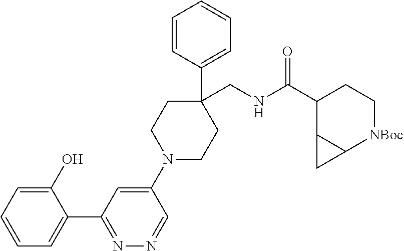 [SMILES: CC(C)(C)OC(=O)N1CCC(C(=O)NCC2(c3ccccc3)CCN(c3cnnc(-c4ccccc4O)c3)CC2)C2CC21]

This intermediate was synthesized as described for Example 104 Step 1, using 2-(tert-butoxycarbonyl)-2-azabicyclo[4.1.0]heptane-5-carboxylic acid in place of 1-(tert-butoxycarbonyl)-9-oxa-1-azaspiro[5.5]undecane-4-carboxylic acid. LCMS C₃₄H₄₁N₅O₄ requires 583.3, found m/z 584.3 [M+H]⁺.

### Step 2: Synthesis of N-((1-(6-(2-hydroxyphenyl)pyridazin-4-yl)-4-phenylpiperidin-4-yl)methyl)-2-azabicyclo[4.1.0]heptane-5-carboxamide

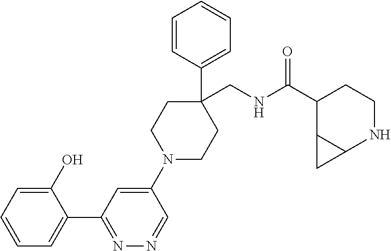 [SMILES: O=C(NCC1(c2ccccc2)CCN(c2cnnc(-c3ccccc3O)c2)CC1)C1CCNC2CC21]

This intermediate was prepared as described for the Example 104 Step 2, using tert-butyl 5-(((1-(6-(2-hydroxyphenyl)pyridazin-4-yl)-4-phenylpiperidin-4-yl)methyl)carbamoyl)-2-azabicyclo[4.1.0]heptane-2-carboxylate in place of tert-butyl 4-(((1-(6-(2-hydroxyphenyl)pyridazin-4-yl)-4-phenylpiperidin-4-yl)methyl)carbamoyl)-9-oxa-1-azaspiro[5.5]undecane-1-carboxylate. LCMS C₂₉H₃₃N₅O₂ requires 483.3, found m/z=484.3 [M+H]⁺.

### Step 3: Synthesis of the Title Compound

The title compound was prepared as described for the Example 104 Step 3, using N-((1-(6-(2-hydroxyphenyl)pyridazin-4-yl)-4-phenylpiperidin-4-yl)methyl)-2-azabicyclo[4.1.0]heptane-5-carboxamide in place of N-((1-(6-(2-hydroxyphenyl)pyridazin-4-yl)-4-phenylpiperidin-4-yl)methyl)-9-oxa-1-azaspiro[5.5]undecane-4-carboxamide (26.9 mg, 64% over three steps). LCMS C₄₇H₅₅N₇O₄ requires 781.4, found m/z=782.3 [M+H]⁺.

### Example 85

### N-{1-[2-(1-{4-[(3R)-2,6-dioxopiperidin-3-yl]-3-fluorophenyl}piperidin-4-yl)ethyl]piperidin-4-yl}-1-[6-(2-hydroxyphenyl)pyridazin-4-yl]-4-(2-methoxyphenyl)-N-methylpiperidine-4-carboxamide

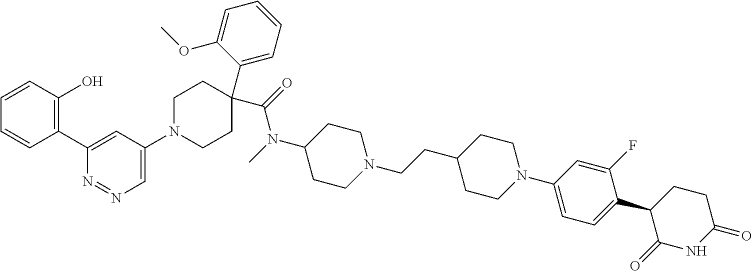 [SMILES: COc1ccccc1C1(C(=O)N(C)C2CCN(CCC3CCN(c4ccc([C@H]5CCC(=O)NC5=O)c(F)c4)CC3)CC2)CCN(c2cnnc(-c3ccccc3O)c2)CC1]

### Step 1: Synthesis of 1-[6-(2-hydroxyphenyl)pyridazin-4-yl]-4-(2-methoxyphenyl)-N-methyl-N-(piperidin-4-yl)piperidine-4-carboxamide

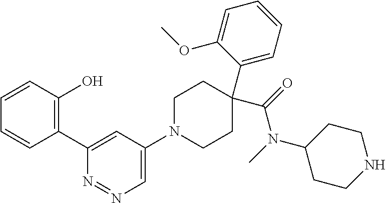 [SMILES: COc1ccccc1C1(C(=O)N(C)C2CCNCC2)CCN(c2cnnc(-c3ccccc3O)c2)CC1]

To a solution of 1-[6-(2-hydroxyphenyl)pyridazin-4-yl]-4-(2-methoxyphenyl)piperidine-4-carboxylic acid (Intermediate 2) (100.5 mg, 0.248 mmol) in 1.6 mL DMA was added tert-butyl 4-(methylamino)piperidine-1-carboxylate (159 mg, 0.743 mmol) and N,N-diisopropylethylamine (129 μL, 0.10 g, 0.743 mmol). Cooled in an ice bath then [(dimethylamino)({[1,2,3]triazolo[4,5-b]pyridin-3-yloxy})methylidene]dimethylazanium; hexafluoro-lambda5-phosphanuide (141.4 mg, 0.372 mmol) was added. Stirred at rt. After 36 days, the reaction was poured into 20 mL water. The resulting solid was collected by filtration, rinsing with more water. Dried the solid under vacuum on the filter. Obtained 135 mg of the Boc protected intermediate. Dissolved in TFA (1.00 mL) and DCM (1.00 mL) and then concentrated by rotovap after 30 min. Redissolved 1:1 ACN/water and lyophilized. Used in the next step as is without further purification. LCMS: C₂₉H₃₅N₅O₂ requires: 501.3. found: m/z=502.4 [M+H]⁺.

### Step 2: Synthesis of the Title Compound

Combined 1-[6-(2-hydroxyphenyl)pyridazin-4-yl]-4-(2-methoxyphenyl)-N-methyl-N-(piperidin-4-yl)piperidine-4-carboxamide; trifluoroacetic acid (30.00 mg, 0.0487 mmol) and 2-(1-{4-[(3R)-2,6-dioxopiperidin-3-yl]-3-fluorophenyl}piperidin-4-yl)acetaldehyde (17.82 mg, 0.0536 mmol) (Synthesized as described in WO2022012622 A1 2022-01-20) in DMA (0.20 mL) and then added sodium triacetoxyborohydride (301.0 mg, 0.146 mmol) and stirred for 0.5 h. Very little product. Added N,N-diisopropylethylamine (42.4 μL, 31.5 mg, 0.244 mmol) and continued stirring for another 0.5 h. Added more STAB (30 mg). Purified the solution directly by RP-HPLC to yield the title compound (2.2 mg, 5.46%) LCMS: C₄₇H₅₆FN₇O₅ requires: 817.4. found: m/z=818.4 [M+H]⁺.

### Example 86

### rac-(r)-n-(1-(2-(4-(4-((2,6-dioxopiperidin-3-yl)amino)phenyl)piperidin-1-yl)acetyl)-4-methylpiperidin-4-yl)-1-(6-(2-hydroxyphenyl)pyridazin-4-yl)-4-phenylpiperidine-4-carboxamide

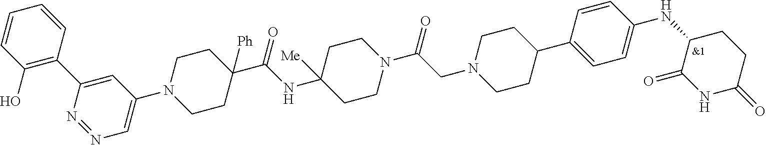 [SMILES: CC1(NC(=O)C2(c3ccccc3)CCN(c3cnnc(-c4ccccc4O)c3)CC2)CCN(C(=O)CN2CCC(c3ccc(N[C@@H]4CCC(=O)NC4=O)cc3)CC2)CC1]

The title compound was synthesized according to Step 3 of Example 108 using intermediates 1-(6-(2-hydroxyphenyl)pyridazin-4-yl)-N-(4-methylpiperidin-4-yl)-4-phenylpiperidine-4-carboxamide and rac-(R)-2-(4-(4-((2,6-dioxopiperidin-3-yl)amino)phenyl)piperidin-1-yl)acetic acid (synthesized as described in WO2021083949 A1 2021-05-06). The solution was injected onto RP-FC and purified with a gradient of 5-95% MeCN in H₂O to furnish the title compound (4.8 mg, 9%). LCMS: C₄₄H₅₃N₉O₄ requires 798.4. found: m/z=799.4 [M+H]⁺.

### Example 87

### rac-N-{1-[2-(1-{4-[(3r)-2,6-dioxopiperidin-3-yl]phenyl}piperidin-4-yl)ethyl]piperidin-4-yl}-1-[6-(2-hydroxyphenyl)pyridazin-4-yl]-N-methyl-4-(1-methyl-1H-pyrazol-5-yl)piperidine-4-carboxamide

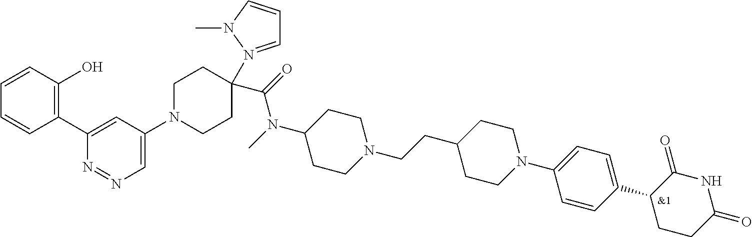

This compound was synthesis according to step 2 of example 98 using 1-[6-(2-hydroxyphenyl)pyridazin-4-yl]-N-methyl-4-(2-methylpyrazol-3-yl)-N-(piperidin-4-yl)piperidine-4-carboxamide and 2-{1-[4-(2,6-dioxopiperidin-3-yl)phenyl]piperidin-4-yl}acetaldehyde. LCMS: C₄₄H₅₅N₉O₄ requires: 773.4. found: m/z=774.4 [M+H]⁺.

### Example 88

### rac-N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N-methyl-4-(3-methyl-1,2-oxazol-5-yl)piperidine-4-carboxamide

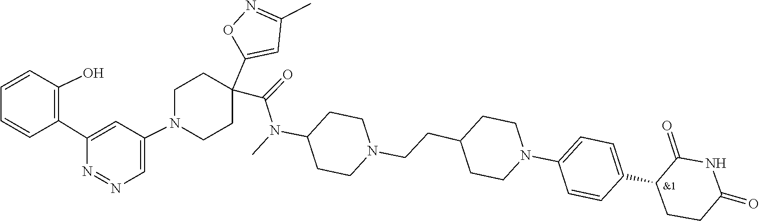 [SMILES: Cc1cc(C2(C(=O)N(C)C3CCN(CCC4CCN(c5ccc([C@H]6CCC(=O)NC6=O)cc5)CC4)CC3)CCN(c3cnnc(-c4ccccc4O)c3)CC2)on1]

This compound was synthesis according to step 2 of Example 98 using 1-[6-(2-hydroxyphenyl)pyridazin-4-yl]-N-methyl-4-(3-methyl-1,2-oxazol-5-yl)-N-(piperidin-4-yl)piperidine-4-carboxamide and 2-{1-[4-(2,6-dioxopiperidin-3-yl)phenyl]piperidin-4-yl}acetaldehyde. LCMS: C₄₄H₅₄N₈O₅ requires: 774.4. found: m/z=775.3 [M+H]⁺.

### Example 89

### rac-N-{1-[(1-{4-[(3R)-2,6-dioxopiperidin-3-yl]phenyl}piperidin-4-yl)methyl]piperidin-4-yl}-1-[6-(2-hydroxyphenyl)pyridazin-4-yl]-N-methyl-4-(1-methyl-1H-pyrazol-4-yl)piperidine-4-carboxamide

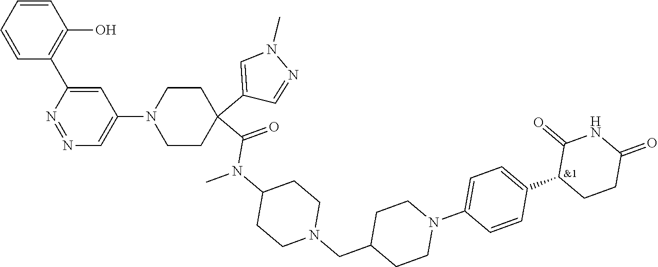

### Step 1: Synthesis of 1-[6-(2-hydroxyphenyl)pyridazin-4-yl]-N-methyl-4-(1-methylpyrazol-4-yl)-N-(piperidin-4-yl)piperidine-4-carboxamide

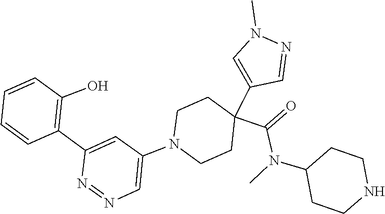 [SMILES: CN(C(=O)C1(c2cnn(C)c2)CCN(c2cnnc(-c3ccccc3O)c2)CC1)C1CCNCC1]

This intermediate was synthesized according to step 1 of Example 97 using 1-[6-(2-hydroxyphenyl)pyridazin-4-yl]-4-(1-methylpyrazol-4-yl)piperidine-4-carboxylic acid (Intermediate 12). LCMS: C₂₆H₃₃N₇O₂ requires: 475.3. found: m/z=476.4 [M+H]⁺.

### Step 2: Synthesis of the Title Compound

This compound was synthesis according to step 2 of example 98 using 1-[6-(2-hydroxyphenyl)pyridazin-4-yl]-N-methyl-4-(1-methylpyrazol-4-yl)-N-(piperidin-4-yl)piperidine-4-carboxamide. LCMS: C₄₃H₅₃N₉O₄ requires: 759.4. found: m/z=760.4 [M+H]⁺.

### Example 90

### rac-N-{1-[(1-{4-[(3R)-2,6-dioxopiperidin-3-yl]phenyl}piperidin-4-yl)methyl]piperidin-4-yl}-1-[6-(2-hydroxyphenyl)pyridazin-4-yl]-N-methyl-4-(3-methyl-1,2-oxazol-5-yl)piperidine-4-carboxamide

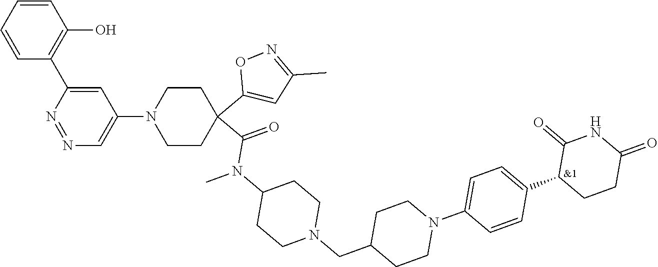

### Step 1: Synthesis of 1-[6-(2-hydroxyphenyl)pyridazin-4-yl]-N-methyl-4-(3-methyl-1,2-oxazol-5-yl)-N-(piperidin-4-yl)piperidine-4-carboxamide

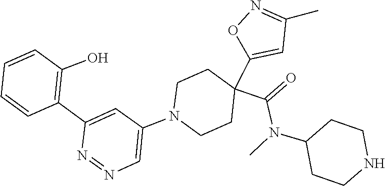 [SMILES: Cc1cc(C2(C(=O)N(C)C3CCNCC3)CCN(c3cnnc(-c4ccccc4O)c3)CC2)on1]

This intermediate was synthesized according to step 1 of Example 98 using 1-[6-(2-hydroxyphenyl)pyridazin-4-yl]-4-(3-methyl-1,2-oxazol-5-yl)piperidine-4-carboxylic acid (Intermediate 13). LCMS: C₂₆H₃₂N₆O₃ requires: 476.3. found: m/z=477.4 [M+H]⁺.

### Step 2: Synthesis of the Title Compound

This compound was synthesis according to step 2 of example 98 using 1-[6-(2-hydroxyphenyl)pyridazin-4-yl]-N-methyl-4-(3-methyl-1,2-oxazol-5-yl)-N-(piperidin-4-yl)piperidine-4-carboxamide. LCMS: C₄₃H₅₂N₈O₅ requires: 760.4. found: m/z=761.4 [M+H]⁺.

### Step 1: Synthesis of 1-[6-(2-hydroxyphenyl)pyridazin-4-yl]-N-methyl-4-(2-methylpyrazol-3-yl)-N-(piperidin-4-yl)piperidine-4-carboxamide

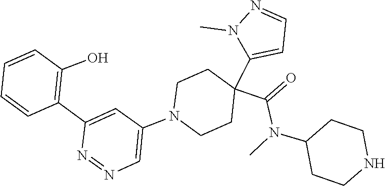 [SMILES: CN(C(=O)C1(c2ccnn2C)CCN(c2cnnc(-c3ccccc3O)c2)CC1)C1CCNCC1]

This intermediate was synthesized according to step 1 of Example 97 using 1-[6-(2-hydroxyphenyl)pyridazin-4-yl]-4-(2-methylpyrazol-3-yl)piperidine-4-carboxylic acid. LCMS: C₂₆H₃₃N₇O₂ requires: 475.3. found: m/z=476.4 [M+H]⁺.

### Example 91

### rac-N-{1-[(1-{4-[(3R)-2,6-dioxopiperidin-3-yl]phenyl}piperidin-4-yl)methyl]piperidin-4-yl}-1-[6-(2-hydroxyphenyl)pyridazin-4-yl]-N-methyl-4-(1-methyl-1H-pyrazol-5-yl)piperidine-4-carboxamide

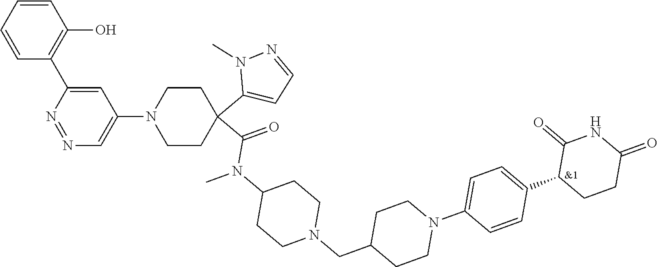

### Step 2: Synthesis of the Title Compound

This compound was synthesis according to step 2 of example 98 using 1-[6-(2-hydroxyphenyl)pyridazin-4-yl]-N-methyl-4-(2-methylpyrazol-3-yl)-N-(piperidin-4-yl)piperidine-4-carboxamide. LCMS: C₄₃H₅₃N₉O₄ requires: 759.4. found: m/z=760.3 [M+H]⁺.

### Example 92

### rac-(3R)-3-(4-{4-[(6-{1-[6-(2-hydroxyphenyl)pyridazin-4-yl]-4-[5-(propan-2-yl)-1,2-oxazol-3-yl]piperidine-4-carbonyl}-2,6-diazaspiro[3.3]heptan-2-yl)methyl]piperidin-1-yl}phenyl)piperidine-2,6-dione

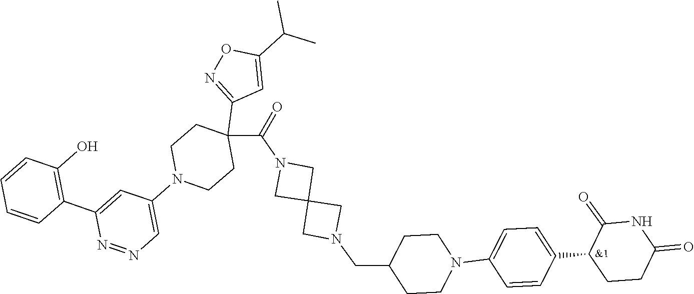 [SMILES: CC(C)c1cc(C2(C(=O)N3CC4(CN(CC5CCN(c6ccc([C@H]7CCC(=O)NC7=O)cc6)CC5)C4)C3)CCN(c3cnnc(-c4ccccc4O)c3)CC2)no1]

### Step 1: Synthesis of 2-[5-(4-{2,6-diazaspiro[3.3]heptane-2-carbonyl}-4-(5-isopropyl-1,2-oxazol-3-yl)piperidin-1-yl)pyridazin-3-yl]phenol

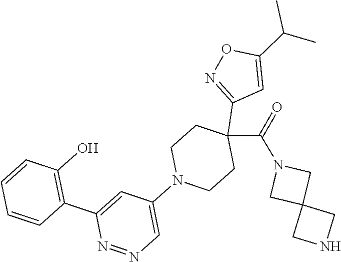 [SMILES: CC(C)c1cc(C2(C(=O)N3CC4(CNC4)C3)CCN(c3cnnc(-c4ccccc4O)c3)CC2)no1]

This intermediate was synthesized according to step 1 of Example 97 using 1-[6-(2-hydroxyphenyl)pyridazin-4-yl]-4-(5-isopropyl-1,2-oxazol-3-yl)piperidine-4-carboxylic acid (Intermediate 10) and tert-butyl 2,6-diazaspiro[3.3]heptane-2-carboxylate. LCMS: C₂₇H₃₇N₆O₃ requires: 488.3. found: m/z=489.4 [M+H]⁺.

### Step 2: Synthesis of the Title Compound

This compound was synthesis according to step 2 of example 98 using 2-[5-(4-{2,6-diazaspiro[3.3]heptane-2-carbonyl}-4-(5-isopropyl-1,2-oxazol-3-yl)piperidin-1-yl)pyridazin-3-yl]phenol. LCMS: C₄₄H₅₂N₈O₅ requires: 772.4. found: m/z=773.3 [M+H]⁺.

### Example 93

### rac-(3R)-3-(4-{4-[(6-{1-[6-(2-hydroxyphenyl)pyridazin-4-yl]-4-(1-methyl-1H-pyrazol-5-yl)piperidine-4-carbonyl}-2,6-diazaspiro[3.3]heptan-2-yl)methyl]piperidin-1-yl}phenyl)piperidine-2,6-dione

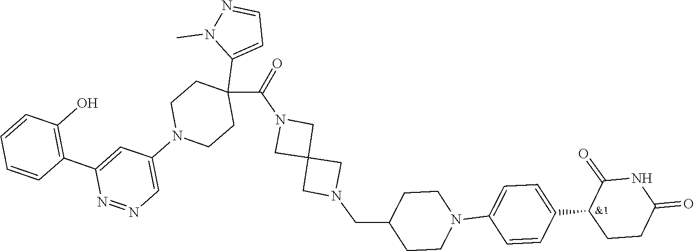 [SMILES: Cn1nccc1C1(C(=O)N2CC3(CN(CC4CCN(c5ccc([C@H]6CCC(=O)NC6=O)cc5)CC4)C3)C2)CCN(c2cnnc(-c3ccccc3O)c2)CC1]

### Step 1: Synthesis of 2-[5-(4-{2,6-diazaspiro[3.3]heptane-2-carbonyl}-4-(2-methylpyrazol-3-yl)piperidin-1-yl)pyridazin-3-yl]phenol

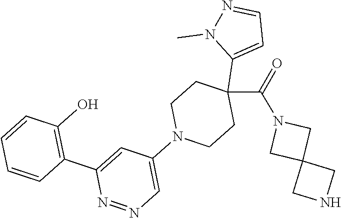 [SMILES: Cn1nccc1C1(C(=O)N2CC3(CNC3)C2)CCN(c2cnnc(-c3ccccc3O)c2)CC1]

This intermediate was synthesized according to step 1 of Example 97 using 1-[6-(2-hydroxyphenyl)pyridazin-4-yl]-4-(2-methylpyrazol-3-yl)piperidine-4-carboxylic acid and tert-butyl 2,6-diazaspiro[3.3]heptane-2-carboxylate. LCMS: C₂₅H₂₉N₇O₂ requires: 459.2. found: m/z=460.2 [M+H]⁺.

### Step 2: Synthesis of the Title Compound

This compound was synthesis according to step 2 of example 98 using 2-[5-(4-{2,6-diazaspiro[3.3]heptane-2-carbonyl}-4-(2-methylpyrazol-3-yl)piperidin-1-yl)pyridazin-3-yl]phenol. LCMS: C₄₂H₄₉N₉O₄ requires: 743.4. found: m/z=744.3 [M+H]⁺.

### Example 94

### rac-N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N-methyl-4-(1-methyl-1H-pyrazol-4-yl)piperidine-4-carboxamide

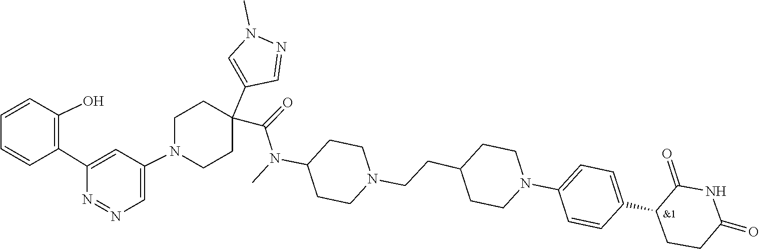 [SMILES: CN(C(=O)C1(c2cnn(C)c2)CCN(c2cnnc(-c3ccccc3O)c2)CC1)C1CCN(CCC2CCN(c3ccc([C@H]4CCC(=O)NC4=O)cc3)CC2)CC1]

This compound was synthesis according to step 2 of example 98 using 1-[6-(2-hydroxyphenyl)pyridazin-4-yl]-N-methyl-4-(1-methylpyrazol-4-yl)-N-(piperidin-4-yl)piperidine-4-carboxamide and 2-{1-[4-(2,6-dioxopiperidin-3-yl)phenyl]piperidin-4-yl}acetaldehyde. LCMS: C₄₄H₅₅N₉O₄ requires: 773.4. found: m/z=774.4 [M+H]⁺.

### Example 95

### rac-(3R)-3-(4-{4-[2-(6-{1-[6-(2-hydroxyphenyl)pyridazin-4-yl]-4-[5-(propan-2-yl)-1,2-oxazol-3-yl]piperidine-4-carbonyl}-2,6-diazaspiro[3.3]heptan-2-yl)ethyl]piperidin-1-yl}phenyl)piperidine-2,6-dione

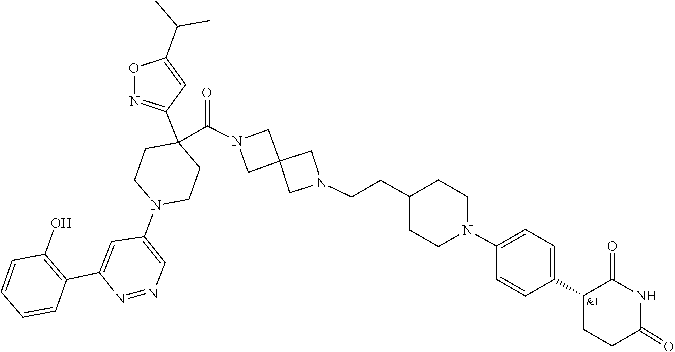 [SMILES: CC(C)c1cc(C2(C(=O)N3CC4(CN(CCC5CCN(c6ccc([C@H]7CCC(=O)NC7=O)cc6)CC5)C4)C3)CCN(c3cnnc(-c4ccccc4O)c3)CC2)no1]

This compound was synthesis according to step 2 of example 98 using 2-[5-(4-{2,6-diazaspiro[3.3]heptane-2-carbonyl}-4-(5-isopropyl-1,2-oxazol-3-yl)piperidin-1-yl)pyridazin-3-yl]phenol and 2-{1-[4-(2,6-dioxopiperidin-3-yl)phenyl]piperidin-4-yl}acetaldehyde. LCMS: C₄₅H₅₄N₈O₅ requires: 786.4. found: m/z=787.4 [M+H]⁺.

### Example 96

### rac-N-{1-[(1-{4-[(3R)-2,6-dioxopiperidin-3-yl]phenyl}piperidin-4-yl)methyl]piperidin-4-yl}-1-[6-(2-hydroxyphenyl)pyridazin-4-yl]-N-methyl-4-(5-methyl-1H-pyrazol-1-yl)piperidine-4-carboxamide

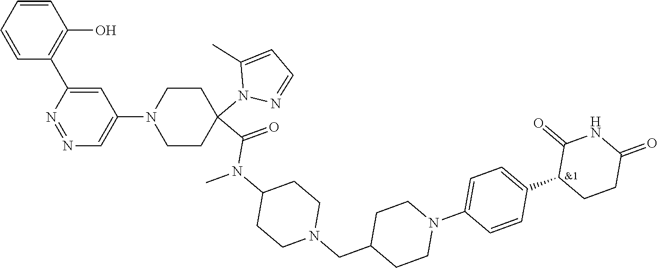

### Step 1: Synthesis of 1-[6-(2-hydroxyphenyl)pyridazin-4-yl]-N-methyl-4-(5-methylpyrazol-1-yl)-N-(piperidin-4-yl)piperidine-4-carboxamide

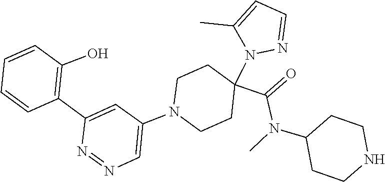 [SMILES: Cc1ccnn1C1(C(=O)N(C)C2CCNCC2)CCN(c2cnnc(-c3ccccc3O)c2)CC1]

This intermediate was synthesized according to step 1 of Example 97 using 1-[6-(2-hydroxyphenyl)pyridazin-4-yl]-4-(5-methylpyrazol-1-yl)piperidine-4-carboxylic acid (Intermediate 14). LCMS: C₂₆H₃₃N₇O₂ requires: 475.3. found: m/z=476.4 [M+H]⁺.

### Step 2: Synthesis of the Title Compound

This compound was synthesis according to step 2 of example 98 using 1-[6-(2-hydroxyphenyl)pyridazin-4-yl]-N-methyl-4-(5-methylpyrazol-1-yl)-N-(piperidin-4-yl)piperidine-4-carboxamide. LCMS: C₄₃H₅₃N₉O₄ requires: 759.4. found: m/z=760.4 [M+H]⁺.

### Example 97

### rac-N-{1-[(1-{4-[(3R)-2,6-dioxopiperidin-3-yl]phenyl}piperidin-4-yl)methyl]piperidin-4-yl}-1-[6-(2-hydroxyphenyl)pyridazin-4-yl]-N-methyl-4-{pyrazolo[1,5-a]pyridin-2-yl}piperidine-4-carboxamide

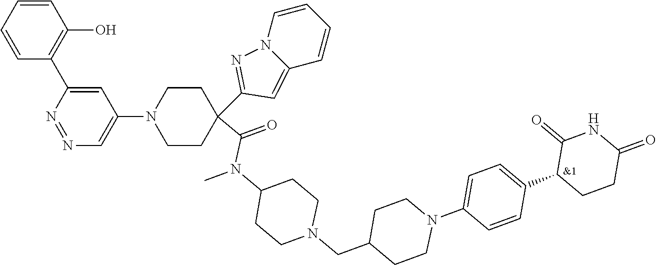 [SMILES: CN(C(=O)C1(c2cc3ccccn3n2)CCN(c2cnnc(-c3ccccc3O)c2)CC1)C1CCN(CC2CCN(c3ccc([C@H]4CCC(=O)NC4=O)cc3)CC2)CC1]

### Step 1: Synthesis of 1-[6-(2-hydroxyphenyl)pyridazin-4-yl]-N-methyl-N-(piperidin-4-yl)-4-{pyrazolo[1,5-a]pyridin-2-yl}piperidine-4-carboxamide

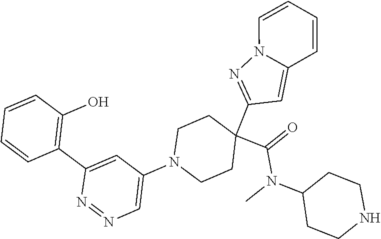 [SMILES: CN(C(=O)C1(c2cc3ccccn3n2)CCN(c2cnnc(-c3ccccc3O)c2)CC1)C1CCNCC1]

To a solution of 1-[6-(2-hydroxyphenyl)pyridazin-4-yl]-4-{pyrazolo[1,5-a]pyridin-2-yl}piperidine-4-carboxylic acid (Intermediate 11) (110 mg, 0.265 mmol) in 1.6 mL DMA was added tert-butyl 4-(methylamino)piperidine-1-carboxylate (170 mg, 0.794 mmol) and N,N-diisopropylethylamine (138.36 μL, 0.10 g, 0.7943 mmol). Cooled in an ice bath then [(dimethylamino)({[1,2,3]triazolo[4,5-b]pyridin-3-yloxy})methylidene]dimethylazanium; hexafluoro-lambda5-phosphanuide (151. mg, 0.3972 mmol) was added. Stirred at rt for 4 days. The reaction mixture was added into 20 mL water and an oily ppt was extracted with EtOAc (2×5 mL). Washed the combined organic layers with brine and passed it through a phase separator. Removed the solvent by rotary evaporation and dried the resulting solid under vacuum. This material was dissolved in DCM (1 mL) and TFA (1 mL) and after 1 hour, it was concentrated by rotary evaporation, and then lyophilized twice from 1:1 ACN/water. Used in the next step as is without further purification. LCMS: C₂₉H₃₃N₇O₂ requires: 511.3. found: m/z=512.4 [M+H]⁺.

### Step 2: Synthesis of the Title Compound

This compound was synthesis according to step 2 of example 98 using 1-[6-(2-hydroxyphenyl)pyridazin-4-yl]-N-methyl-N-(piperidin-4-yl)-4-{pyrazolo[1,5-a]pyridin-2-yl}piperidine-4-carboxamide. LCMS: C₄₆H₅₃N₉O₄ requires: 795.4. found: m/z=796.3 [M+H]⁺.

### Example 98

### rac-N-{1-[(1-{4-[(3R)-2,6-dioxopiperidin-3-yl]phenyl}piperidin-4-yl)methyl]piperidin-4-yl}-1-[6-(2-hydroxyphenyl)pyridazin-4-yl]-N-methyl-4-[5-(propan-2-yl)-1,2-oxazol-3-yl]piperidine-4-carboxamide

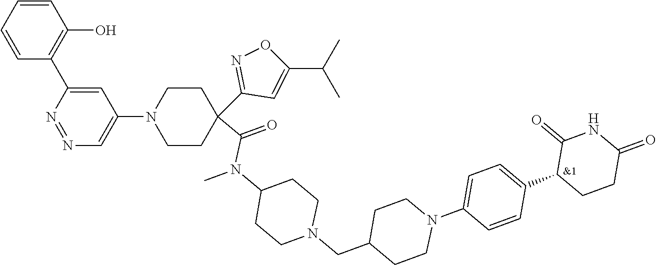 [SMILES: CC(C)c1cc(C2(C(=O)N(C)C3CCN(CC4CCN(c5ccc([C@H]6CCC(=O)NC6=O)cc5)CC4)CC3)CCN(c3cnnc(-c4ccccc4O)c3)CC2)no1]

### Step 1: Synthesis of 1-[6-(2-hydroxyphenyl)pyridazin-4-yl]-4-(5-isopropyl-1,2-oxazol-3-yl)-N-methyl-N-(piperidin-4-yl)piperidine-4-carboxamide

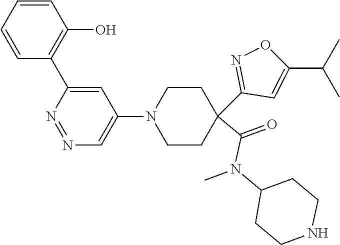 [SMILES: CC(C)c1cc(C2(C(=O)N(C)C3CCNCC3)CCN(c3cnnc(-c4ccccc4O)c3)CC2)no1]

To a solution of 1-[6-(2-hydroxyphenyl)pyridazin-4-yl]-4-(5-isopropyl-1,2-oxazol-3-yl)piperidine-4-carboxylic acid (104 mg, 0.255 mmol) in 1.6 mL DMA was added tert-butyl 4-(methylamino)piperidine-1-carboxylate (163.7 mg, 0.764 mmol) and N,N-diisopropylethylamine (133 μL, 0.10 g, 0.764 mmol). Cooled in an ice bath then [(dimethylamino)({[1,2,3]triazolo[4,5-b]pyridin-3-yloxy})methylidene]dimethylazanium; hexafluoro-lambda5-phosphanuide (145 mg, 0.382 mmol) was added. Stirred at rt for 4 days. Precipitated out the product by adding the reaction mixture into 20 mL water. Filtered to collect the solid, rinsing with water (2×5 mL). Dried the solid under vacuum. This material was dissolved in DCM (1 mL) and TFA (1 mL) and after 1 hour, it was concentrated by rotary evaporation, and then lyophilized twice from 1:1 ACN/water. Used in the next step as is without further purification. LCMS: C₂₈H₃₆N₆O₃ requires: 504.3. found: m/z=505.4 [M+H]⁺.

### Step 2: Synthesis of the Title Compound

Combined solutions of 1-(6-(2-hydroxyphenyl)pyridazin-4-yl)-4-(5-isopropylisoxazol-3-yl)-N-methyl-N-(piperidin-4-yl)piperidine-4-carboxamide 2,2,2-trifluoroacetate (10.5 mg, 0.0170 mmol) in DMA (0.1 mL) and rac-(R)-1-(4-(2,6-dioxopiperidin-3-yl)phenyl)piperidine-4-carbaldehyde (5.62 mg, 0.0187 mmol) in DMA (0.1 mL) together with N,N-diisopropylethylamine (14.82 μL, 0.01 g, 0.0851 mmol). Then added a solution of sodium triacetoxyborohydride (10.82 mg, 0.0511 mmol) in DMA (0.2 mL) and stirred for 2 h. Purified the solution directly by RP-HPLC to yield the title compound (6.0 g, 27.8%). LCMS: C₄₅H₅₆N₈O₅ requires: 788.4. found: m/z=789.4 [M+H]⁺.

### Example 99

### rac-(r)-n-(1-((1-(5-((2,6-dioxopiperidin-3-yl)amino)pyridin-2-yl)piperidin-4-yl)methyl)-4-methylpiperidin-4-yl)-1-(6-(2-hydroxyphenyl)pyridazin-4-yl)-4-phenylpiperidine-4-carboxamide

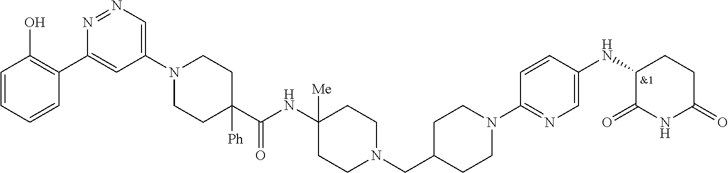 [SMILES: CC1(NC(=O)C2(c3ccccc3)CCN(c3cnnc(-c4ccccc4O)c3)CC2)CCN(CC2CCN(c3ccc(N[C@@H]4CCC(=O)NC4=O)cn3)CC2)CC1]

### Step 1: Synthesis of 1-(6-(2-hydroxyphenyl)pyridazin-4-yl)-N-(4-methylpiperidin-4-yl)-4-phenylpiperidine-4-carboxamide

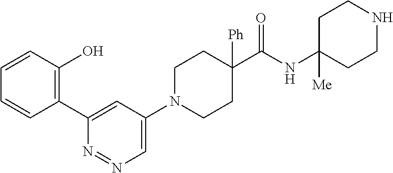 [SMILES: CC1(NC(=O)C2(c3ccccc3)CCN(c3cnnc(-c4ccccc4O)c3)CC2)CCNCC1]

This intermediate was synthesized according to Step 1 of Example 108 using tert-butyl 4-amino-4-methylpiperidine-1-carboxylate. LCMS: C₂₈H₃₃N₅O₂ requires 471.3. found: m/z=472.3 [M+H]⁺.

### Step 2: Synthesis of rac-(R)-1-(5-((2,6-dioxopiperidin-3-yl)amino)pyridin-2-yl)piperidine-4-carbaldehyde

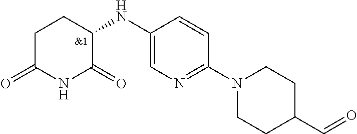 [SMILES: O=CC1CCN(c2ccc(N[C@H]3CCC(=O)NC3=O)cn2)CC1]

To a solution of 3-({6-[4-(hydroxymethyl)piperidin-1-yl]pyridin-3-yl}amino)piperidine-2,6-dione (30.0 mg, 0.094 mmol) in DMSO (0.40 mL) was slowly added triethylamine (65.31 μL, 0.05 g, 0.471 mmol) and sulfur trioxide pyridine complex (30.0 mg, 0.189 mmol) and the reaction mixture was stirred at room temperature until complete by LCMS. The product was carried to the next step as a solution in DMSO. LCMS: C₁₆H₂₀N₄O₃ requires 316.2. found: m/z=317.4 [M+H]⁺.

### Step 3: Synthesis of the Title Compound

The title compound was synthesized according to Step 3 of Example 145 using 1-(6-(2-hydroxyphenyl)pyridazin-4-yl)-N-(4-methylpiperidin-4-yl)-4-phenylpiperidine-4-carboxamide and rac-(R)-1-(5-((2,6-dioxopiperidin-3-yl)amino)pyridin-2-yl)piperidine-4-carbaldehyde. The solution was injected onto RP-FC and purified with a gradient of 5-95% MeCN in H₂O to furnish the title compound (3.3 mg, 6%). LCMS: C₄₄H₅₃N₉O₄ requires 771.4. found: m/z=772.3 [M+H]⁺.

### Example 100

### N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-4-phenyl-n-propylpiperidine-4-carboxamide

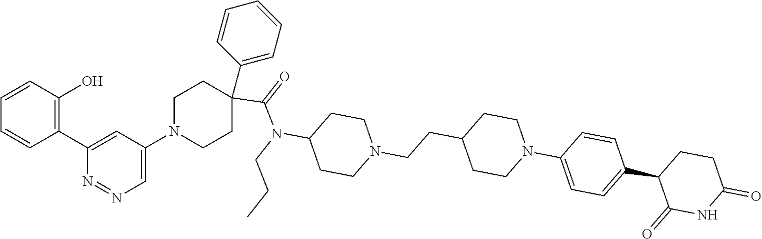 [SMILES: CCCN(C(=O)C1(c2ccccc2)CCN(c2cnnc(-c3ccccc3O)c2)CC1)C1CCN(CCC2CCN(c3ccc([C@H]4CCC(=O)NC4=O)cc3)CC2)CC1]

Combined 1-[6-(2-hydroxyphenyl)pyridazin-4-yl]-4-phenyl-N-(piperidin-4-yl)-N-propylpiperidine-4-carboxamide (Intermediate 26); trifluoroacetic acid (20.20 mg, 0.0329 mmol) and 2-(1-{4-[(3R)-2,6-dioxopiperidin-3-yl]phenyl}piperidin-4-yl)acetaldehyde (11.38 mg, 0.0362 mmol) in DMA (0.20 mL) and then added sodium triacetoxyborohydride (20.93 mg, 0.0987 mmol) and stirred for 2 h. Purified the solution directly by RP-HPLC to yield the title compound (15.9 mg, 57.62%) LCMS: C₄₈H₅₉N₇O₄ requires: 797.5. found: m/z=798.3 [M+H]⁺.

### Example 101

### rac-®-3-(3-(4-((2-(1-(6-(2-hydroxyphenyl)pyridazin-4-yl)-4-phenylpiperidine-4-carbonyl)-2,7-diazaspiro[3.5]nonan-7-yl)methyl)piperidin-1-yl)phenyl)piperidine-2,6-dione

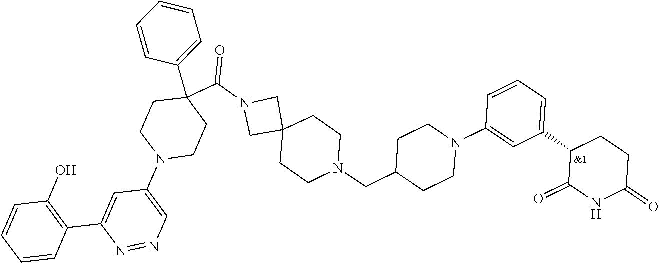 [SMILES: O=C1CC[C@@H](c2cccc(N3CCC(CN4CCC5(CC4)CN(C(=O)C4(c6ccccc6)CCN(c6cnnc(-c7ccccc7O)c6)CC4)C5)CC3)c2)C(=O)N1]

### Step 1: Synthesis of the Title Compound

The title compound was prepared in a manner similar to Example 102 Step 1, using (1-(6-(2-hydroxyphenyl)pyridazin-4-yl)-4-phenylpiperidin-4-yl)(2,7-diazaspiro[3.5]nonan-2-yl)methanone (synthesized as described in WO2022235698 A1 2022-11-10) in place of 1-(6-(2-hydroxyphenyl)pyridazin-4-yl)-N-methyl-4-phenyl-N-(piperidin-4-yl)piperidine-4-carboxamide (7.7 mgs, 52% yield). LCMS C₄₆H₅₃N₇O₄ requires 767.4, found m/z=768.4 [M+H]⁺.

### Example 102

### rac-(r)-n-(1-((1-(3-(2,6-dioxopiperidin-3-yl)phenyl)piperidin-4-yl)methyl)piperidin-4-yl)-1-(6-(2-hydroxyphenyl)pyridazin-4-yl)-n-methyl-4-phenylpiperidine-4-carboxamide

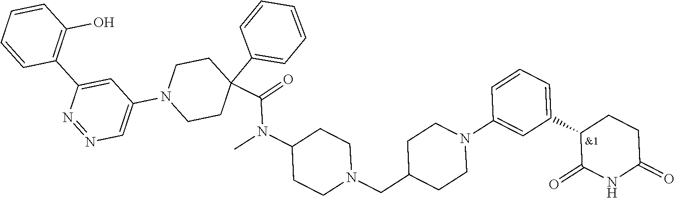 [SMILES: CN(C(=O)C1(c2ccccc2)CCN(c2cnnc(-c3ccccc3O)c2)CC1)C1CCN(CC2CCN(c3cccc([C@@H]4CCC(=O)NC4=O)c3)CC2)CC1]

### Step 1: Synthesis of Title Compound

To a vial containing 1-(6-(2-hydroxyphenyl)pyridazin-4-yl)-N-methyl-4-phenyl-N-(piperidin-4-yl)piperidine-4-carboxamide (6.9 mg, 0.015 mmol) was added rac-(R)-1-(3-(2,6-dioxopiperidin-3-yl)phenyl)piperidine-4-carbaldehyde (4.8 mgs, 0.016 mmol), DMA (0.2 mL), and DIPEA (12.8 μL, 0.073 mmol), and the mixture was allowed to stir for 30 minutes at RT. After 30 minutes, sodium triacetoxyborohydride (9.3 mgs, 0.044 mmol) was added in one portion to the mixture and the reaction was allowed to stir until completion as judged by LCMS. Once complete, the reaction mixture was directly injected onto RP-FC and purified with a gradient of 5-95% MeCN in H₂O to furnish the title compound (8.7 mgs, 78% yield). LCMS C₄₅H₅₃N₇O₄ requires 755.4, found m/z=756.4 [M+H]⁺.

### Example 103

### rac-(r)-1-(2-(1-(4-(2,6-dioxopiperidin-3-yl)phenyl)piperidin-4-yl)ethyl)-n-((1-(6-(2-hydroxyphenyl)pyridazin-4-yl)-4-phenylpiperidin-4-yl)methyl)-4-phenoxypiperidine-4-carboxamide

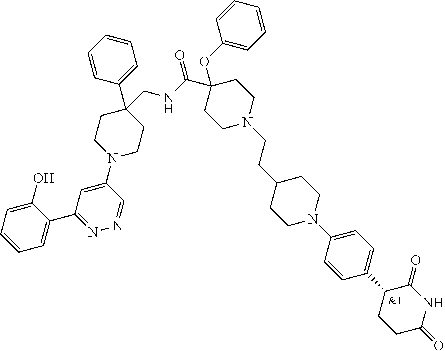 [SMILES: O=C1CC[C@H](c2ccc(N3CCC(CCN4CCC(Oc5ccccc5)(C(=O)NCC5(c6ccccc6)CCN(c6cnnc(-c7ccccc7O)c6)CC5)CC4)CC3)cc2)C(=O)N1]

### Step 1: Synthesis of tert-butyl 4-(((1-(6-(2-hydroxyphenyl)pyridazin-4-yl)-4-phenylpiperidin-4-yl)methyl)carbamoyl)-4-phenoxypiperidine-1-carboxylate

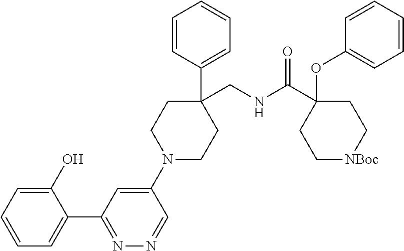 [SMILES: CC(C)(C)OC(=O)N1CCC(Oc2ccccc2)(C(=O)NCC2(c3ccccc3)CCN(c3cnnc(-c4ccccc4O)c3)CC2)CC1]

This intermediate was synthesized as described for Example 104 Step 1 using 1-(tert-butoxycarbonyl)-4-phenoxypiperidine-4-carboxylic acid in place of 1-(tert-butoxycarbonyl)-9-oxa-1-azaspiro[5.5]undecane-4-carboxylic acid. LCMS C₃₉H₄₅N₅O₅ requires 663.3, found m/z=664.3 [M+H]⁺.

### Step 2: Synthesis of N-((1-(6-(2-hydroxyphenyl)pyridazin-4-yl)-4-phenylpiperidin-4-yl)methyl)-4-phenoxypiperidine-4-carboxamide

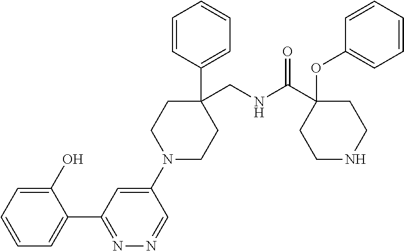 [SMILES: O=C(NCC1(c2ccccc2)CCN(c2cnnc(-c3ccccc3O)c2)CC1)C1(Oc2ccccc2)CCNCC1]

This intermediate was synthesized as described for Example 104 Step 2, using tert-butyl 4-(((1-(6-(2-hydroxyphenyl)pyridazin-4-yl)-4-phenylpiperidin-4-yl)methyl)carbamoyl)-4-phenoxypiperidine-1-carboxylate in place of tert-butyl 4-(((1-(6-(2-hydroxyphenyl)pyridazin-4-yl)-4-phenylpiperidin-4-yl)methyl)carbamoyl)-9-oxa-1-azaspiro[5.5]undecane-1-carboxylate. LCMS C₃₄H₃₇N₅O₃ requires 563.3, found m/z=564.4 [M+H]⁺.

### Step 3: Synthesis of the Title Compound

The title compound was synthesized as described for Example 104 Step 3, using N-((1-(6-(2-hydroxyphenyl)pyridazin-4-yl)-4-phenylpiperidin-4-yl)methyl)-4-phenoxypiperidine-4-carboxamide in place of N-((1-(6-(2-hydroxyphenyl)pyridazin-4-yl)-4-phenylpiperidin-4-yl)methyl)-9-oxa-1-azaspiro[5.5]undecane-4-carboxamide (13.9 mg, 26% yield over three steps). LCMS C₅₂H₅₉N₇O₅ requires 861.4, found m/z=862.4 [M+H]⁺.

### Example 104

### rac-1-(2-(1-(4-((r)-2,6-dioxopiperidin-3-yl)phenyl)piperidin-4-yl)ethyl)-n-((1-(6-(2-hydroxyphenyl)pyridazin-4-yl)-4-phenylpiperidin-4-yl)methyl)-9-oxa-1-azaspiro[5.5]undecane-4-carboxamide

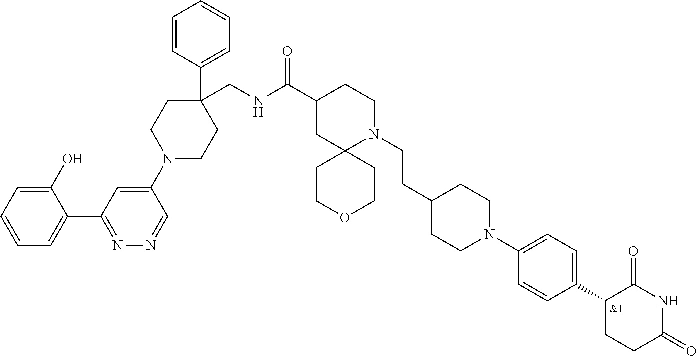 [SMILES: O=C1CC[C@H](c2ccc(N3CCC(CCN4CCC(C(=O)NCC5(c6ccccc6)CCN(c6cnnc(-c7ccccc7O)c6)CC5)CC45CCOCC5)CC3)cc2)C(=O)N1]

### Step 1: Synthesis of tert-butyl 4-(((1-(6-(2-hydroxyphenyl)pyridazin-4-yl)-4-phenylpiperidin-4-yl)methyl)carbamoyl)-9-oxa-1-azaspiro[5.5]undecane-1-carboxylate

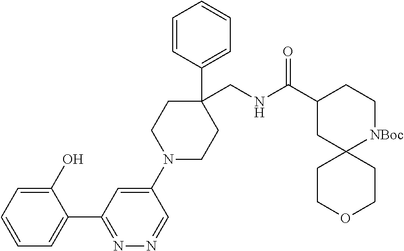 [SMILES: CC(C)(C)OC(=O)N1CCC(C(=O)NCC2(c3ccccc3)CCN(c3cnnc(-c4ccccc4O)c3)CC2)CC12CCOCC2]

This intermediate was synthesized as described for the Example 146 Step 2, using 2-(5-(4-(aminomethyl)-4-phenylpiperidin-1-yl)pyridazin-3-yl)phenol (Intermediate 15) in place of 2-(5-(4-((methylamino)methyl)-4-phenylpiperidin-1-yl)pyridazin-3-yl)phenol and 1-(tert-butoxycarbonyl)-9-oxa-1-azaspiro[5.5]undecane-4-carboxylic acid in place of 5-(tert-butoxycarbonyl)-5-azaspiro[3.5]nonane-8-carboxylic acid. LCMS C₃₇H₄₇N₅O₅ requires 641.4, found m/z=642.4 [M+H]⁺.

### Step 2: Synthesis of N-((1-(6-(2-hydroxyphenyl)pyridazin-4-yl)-4-phenylpiperidin-4-yl)methyl)-9-oxa-1-azaspiro[5.5]undecane-4-carboxamide

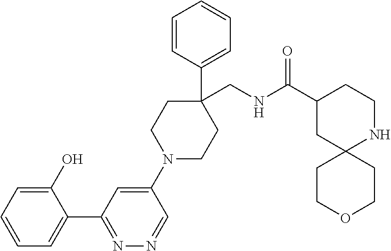 [SMILES: O=C(NCC1(c2ccccc2)CCN(c2cnnc(-c3ccccc3O)c2)CC1)C1CCNC2(CCOCC2)C1]

This intermediate was synthesized as described for the Example 146 Step 3, using tert-butyl 4-(((1-(6-(2-hydroxyphenyl)pyridazin-4-yl)-4-phenylpiperidin-4-yl)methyl)carbamoyl)-9-oxa-1-azaspiro[5.5]undecane-1-carboxylate in place of tert-butyl 8-(((1-(6-(2-hydroxyphenyl)pyridazin-4-yl)-4-phenylpiperidin-4-yl)methyl)(methyl)carbamoyl)-5-azaspiro[3.5]nonane-5-carboxylate. LCMS C₃₂H₃₉N₅O₃ requires 541.3, found m/z=542.3 [M+H]⁺.

### Step 3: Synthesis of the Title Compound

The title compound was synthesized as described for the Example 146 Step 4, using N-((1-(6-(2-hydroxyphenyl)pyridazin-4-yl)-4-phenylpiperidin-4-yl)methyl)-9-oxa-1-azaspiro[5.5]undecane-4-carboxamide in place of N-((1-(6-(2-hydroxyphenyl)pyridazin-4-yl)-4-phenylpiperidin-4-yl)methyl)-N-methyl-5-azaspiro[3.5]nonane-8-carboxamide (14.6 mgs, 29% yield over 3 steps). LCMS C₅₀H₆₁N₇O₅ requires 839.4, found m/z=840.4 [M+H]⁺.

### Example 105

### rac-(r)-1-(2-(1-(4-(2,6-dioxopiperidin-3-yl)phenyl)piperidin-4-yl)ethyl)-n-((1-(6-(2-hydroxyphenyl)pyridazin-4-yl)-4-phenylpiperidin-4-yl)methyl)-4-(2,2,2-trifluoroethoxy)piperidine-4-carboxamide

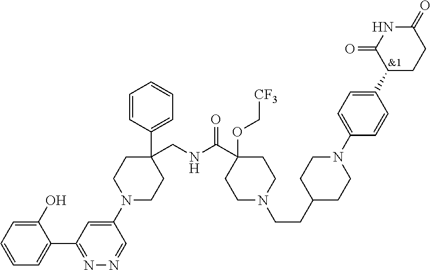 [SMILES: O=C1CC[C@H](c2ccc(N3CCC(CCN4CCC(OCC(F)(F)F)(C(=O)NCC5(c6ccccc6)CCN(c6cnnc(-c7ccccc7O)c6)CC5)CC4)CC3)cc2)C(=O)N1]

### Step 1: Synthesis of tert-butyl 4-(((1-(6-(2-hydroxyphenyl)pyridazin-4-yl)-4-phenylpiperidin-4-yl)methyl)carbamoyl)-4-(2,2,2-trifluoroethoxy)piperidine-1-carboxylate

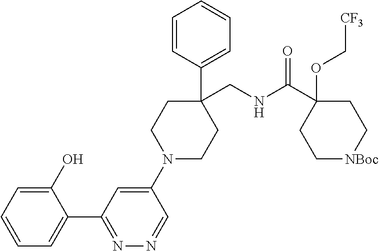 [SMILES: CC(C)(C)OC(=O)N1CCC(OCC(F)(F)F)(C(=O)NCC2(c3ccccc3)CCN(c3cnnc(-c4ccccc4O)c3)CC2)CC1]

This intermediate was synthesized as described for Example 104 Step 1, using 1-(tert-butoxycarbonyl)-4-(2,2,2-trifluoroethoxy)piperidine-4-carboxylic acid in place of 1-(tert-butoxycarbonyl)-9-oxa-1-azaspiro[5.5]undecane-4-carboxylic acid. LCMS C₃₅H₄₂F₃N₅O₅ requires 669.3, found m/z=670.3 [M+H]⁺.

### Step 2: Synthesis of N-((1-(6-(2-hydroxyphenyl)pyridazin-4-yl)-4-phenylpiperidin-4-yl)methyl)-4-(2,2,2-trifluoroethoxy)piperidine-4-carboxamide

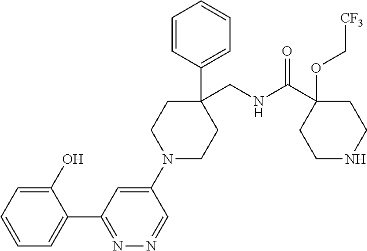 [SMILES: O=C(NCC1(c2ccccc2)CCN(c2cnnc(-c3ccccc3O)c2)CC1)C1(OCC(F)(F)F)CCNCC1]

This intermediate was synthesized as described for Example 104 Step 2, using tert-butyl 4-(((1-(6-(2-hydroxyphenyl)pyridazin-4-yl)-4-phenylpiperidin-4-yl)methyl)carbamoyl)-4-(2,2,2-trifluoroethoxy)piperidine-1-carboxylate in place of tert-butyl 4-(((1-(6-(2-hydroxyphenyl)pyridazin-4-yl)-4-phenylpiperidin-4-yl)methyl)carbamoyl)-9-oxa-1-azaspiro[5.5]undecane-1-carboxylate. LCMS C₃₀H₃₄F₃N₅O₃ requires 569.3, found m/z=570.3 [M+H]⁺.

### Step 3: Synthesis of the Title Compound

The title compound was synthesized as described for Example 104 Step 3, using N-((1-(6-(2-hydroxyphenyl)pyridazin-4-yl)-4-phenylpiperidin-4-yl)methyl)-4-(2,2,2-trifluoroethoxy)piperidine-4-carboxamide in place of N-((1-(6-(2-hydroxyphenyl)pyridazin-4-yl)-4-phenylpiperidin-4-yl)methyl)-9-oxa-1-azaspiro[5.5]undecane-4-carboxamide (23.7 mg, 46% yield over three steps). LCMS C₄₈H₅₆F₃N₇O₅ requires 867.3, found m/z=868.3 [M+H]⁺.

### Example 106

### (3R)-3-{4-[(3RS)-3-(6-{1-[6-(2-hydroxyphenyl)pyridazin-4-yl]-4-(2-methoxyphenyl)piperidine-4-carbonyl}-2,6-diazaspiro[3.3]heptan-2-yl)-1-oxa-8-azaspiro[4.5]decan-8-yl]phenyl}piperidine-2,6-dione

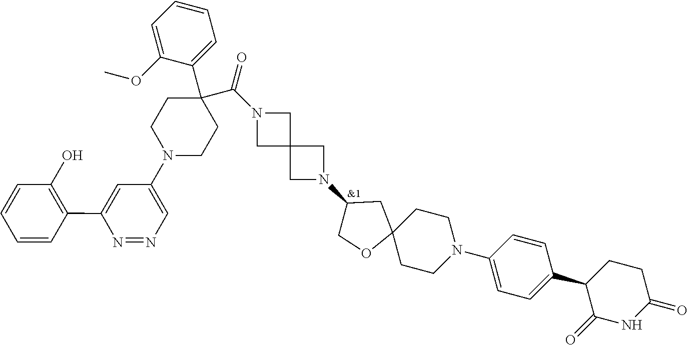 [SMILES: COc1ccccc1C1(C(=O)N2CC3(C2)CN([C@@H]2COC4(CCN(c5ccc([C@H]6CCC(=O)NC6=O)cc5)CC4)C2)C3)CCN(c2cnnc(-c3ccccc3O)c2)CC1]

### Synthesis of 2-[5-(4-{2,6-diazaspiro[3.3]heptane-2-carbonyl}-4-(2-methoxyphenyl)piperidin-1-yl)pyridazin-3-yl]phenol

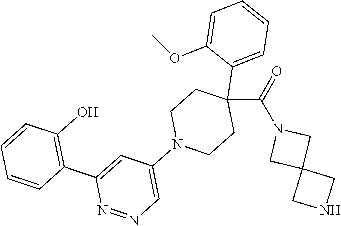 [SMILES: COc1ccccc1C1(C(=O)N2CC3(CNC3)C2)CCN(c2cnnc(-c3ccccc3O)c2)CC1]

A solution of 1-[6-(2-hydroxyphenyl)pyridazin-4-yl]-4-(2-methoxyphenyl)piperidine-4-carboxylic acid (Intermediate 2) (15.00 mg, 0.0370 mmol) in 0.2 mL DMA was added to tert-butyl 2,6-diazaspiro[3.3]heptane-2-carboxylate (8.07 mg, 0.0407 mmol) with N,N-diisopropylethylamine (19.33 μL, 0.01 g, 0.1110 mmol) and then [(dimethylamino)({[1,2,3]triazolo[4,5-b]pyridin-3-yloxy})methylidene]dimethylazanium; hexafluoro-lambda5-phosphanuide (16.88 mg, 0.0444 mmol) was added as a solution in 0.2 mL DMA. Stirred at rt. Diluted with DCM (3 mL) and washed with 1M citric acid (2×4 mL) and bicarb (1×4 ml), then passed through a phase separator. Added TFA (2 mL), then after 45 min, concentrated to dryness. Dissolved in 1:1 ACN/water (2 mL) and dried on the lyophilizer. Used in the next step as is without further purification. LCMS: C₂₈H₃₁N₅O₃ requires: 485.2. found: m/z=486.6 [M+H]⁺.

### Step 3: Synthesis of the Title Compound

Combined 2-[5-(4-{2,6-diazaspiro[3.3]heptane-2-carbonyl}-4-(2-methoxyphenyl)piperidin-1-yl)pyridazin-3-yl]phenol; trifluoroacetic acid (21.60 mg, 0.0351 mmol) and (3R)-3-(4-{3-oxo-1-oxa-8-azaspiro[4.5]decan-8-yl}phenyl)piperidine-2,6-dione (13.21 mg, 0.0386 mmol) in DMA (0.30 mL) and then added sodium triacetoxyborohydride (22.31 mg, 0.1053 mmol) and stirred overnight.

Purified the solution directly by RP-HPLC to yield the title compound (0.0057 g, 19.97%). LCMS: C₄₇H₅₃N₇O₆ requires: 811.4. found: m/z=812.4 [M+H]⁺.

### Example 107

### (r)-1-(2-(1-(4-((rs)-2,6-dioxopiperidin-3-yl)phenyl)piperidin-4-yl)ethyl)-n-((1-(6-(2-hydroxyphenyl)pyridazin-4-yl)-4-phenylpiperidin-4-yl)methyl)pyrrolidine-2-carboxamide

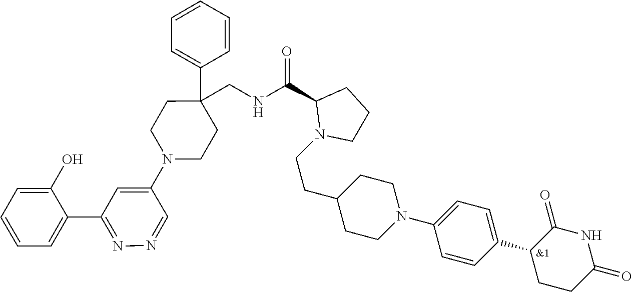 [SMILES: O=C1CC[C@H](c2ccc(N3CCC(CCN4CCC[C@@H]4C(=O)NCC4(c5ccccc5)CCN(c5cnnc(-c6ccccc6O)c5)CC4)CC3)cc2)C(=O)N1]

### Step 1: Synthesis of tert-butyl (R)-2-(((1-(6-(2-hydroxyphenyl)pyridazin-4-yl)-4-phenylpiperidin-4-yl)methyl)carbamoyl)pyrrolidine-1-carboxylate

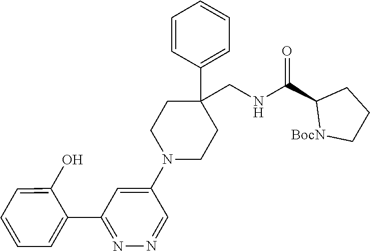 [SMILES: CC(C)(C)OC(=O)N1CCC[C@@H]1C(=O)NCC1(c2ccccc2)CCN(c2cnnc(-c3ccccc3O)c2)CC1]

This intermediate was synthesized as described for the Example 104 Step 1, using (tert-butoxycarbonyl)-D-proline in place of 1-(tert-butoxycarbonyl)-9-oxa-1-azaspiro[5.5]undecane-4-carboxylic acid. LCMS C₃₂H₃₉N₅O₄ requires 557.3, found 558.3 [M+H]⁺.

### Step 2: Synthesis of (R)-N-((1-(6-(2-hydroxyphenyl)pyridazin-4-yl)-4-phenylpiperidin-4-yl)methyl)pyrrolidine-2-carboxamide

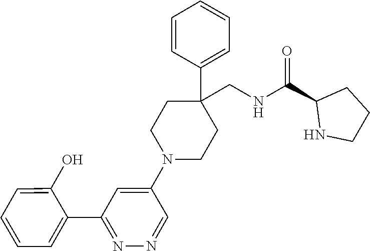 [SMILES: O=C(NCC1(c2ccccc2)CCN(c2cnnc(-c3ccccc3O)c2)CC1)[C@H]1CCCN1]

This intermediate was synthesized as described for the Example 104 Step 2, using tert-butyl (R)-2-(((1-(6-(2-hydroxyphenyl)pyridazin-4-yl)-4-phenylpiperidin-4-yl)methyl)carbamoyl)pyrrolidine-1-carboxylate in place of tert-butyl 4-(((1-(6-(2-hydroxyphenyl)pyridazin-4-yl)-4-phenylpiperidin-4-yl)methyl)carbamoyl)-9-oxa-1-azaspiro[5.5]undecane-1-carboxylate. LCMS C₂₇H₃₁N₅O₂ requires 457.3, found m/z 458.3 [M+H]⁺.

### Step 3: Synthesis of the Title Compound

The title compound was synthesized as described for the Example 104 Step 3, using (R)-N-((1-(6-(2-hydroxyphenyl)pyridazin-4-yl)-4-phenylpiperidin-4-yl)methyl)pyrrolidine-2-carboxamide in place of N-((1-(6-(2-hydroxyphenyl)pyridazin-4-yl)-4-phenylpiperidin-4-yl)methyl)-9-oxa-1-azaspiro[5.5]undecane-4-carboxamide (9.0 mg, 61% yield over three steps). LCMS C₄₅H₅₃N₇O₄ requires 755.4, found m/z 756.5 [M+H]⁺.

### Example 108

### rac-(r)-3-((4-(1-(2-(2-(1-(6-(2-hydroxyphenyl)pyridazin-4-yl)-4-phenylpiperidine-4-carbonyl)-2,7-diazaspiro[3.5]nonan-7-yl)-2-oxoethyl)piperidin-4-yl)phenyl)amino)piperidine-2,6-dione

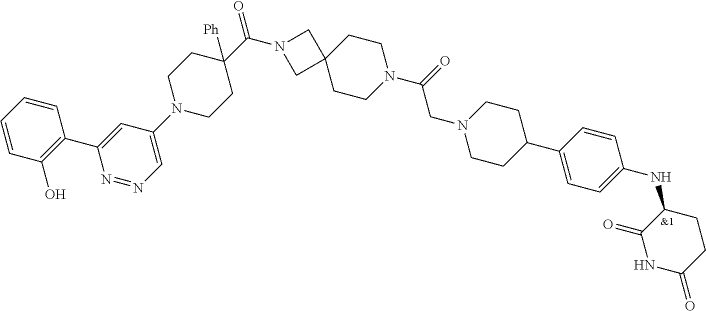 [SMILES: O=C1CC[C@H](Nc2ccc(C3CCN(CC(=O)N4CCC5(CC4)CN(C(=O)C4(c6ccccc6)CCN(c6cnnc(-c7ccccc7O)c6)CC4)C5)CC3)cc2)C(=O)N1]

Step 1: Synthesis of (1-(6-(2-hydroxyphenyl)pyridazin-4-yl)-4-phenylpiperidin-4-yl)(2,7-diazaspiro[3.5]nonan-2-yl)methanone

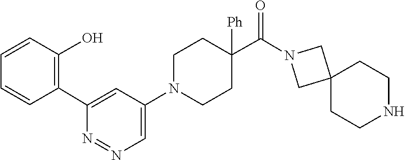 [SMILES: O=C(N1CC2(CCNCC2)C1)C1(c2ccccc2)CCN(c2cnnc(-c3ccccc3O)c2)CC1]

To a solution of 1-[6-(2-hydroxyphenyl)pyridazin-4-yl]-4-phenylpiperidine-4-carboxylic acid (200.00 mg, 0.5327 mmol) and tert-butyl 2,7-diazaspiro[3.5]nonane-7-carboxylate (128.03 mg, 0.6393 mmol) in DMA (1.20 mL) was added N,N-diisopropylethylamine (279.13 μL, 0.21 g, 1.5982 mmol). Then, [(dimethylamino)({[1,2,3]triazolo[4,5-b]pyridin-3-yloxy})methylidene]dimethylazanium; hexafluoro-lambda5-phosphanuide (243.07 mg, 0.6393 mmol) was added and the reaction mixture was stirred until complete by LCMS. Then, it was concentrated to dryness and the crude residue was dissolved in a mixture of MeOH (2.00 mL) and 4N HCl in dioxane (0.80 mL). The reaction mixture was stirred until complete by LCMS. Then, it was concentrated to dryness and used in the next step without further purification. LCMS: C₂₉H₃₃N₅O₂ requires 483.3. found: m/z=484.1 [M+H]⁺.

### Step 2: Synthesis of rac-(R)-2-(4-(4-((2,6-dioxopiperidin-3-yl)amino)phenyl)piperidin-1-yl)acetic acid

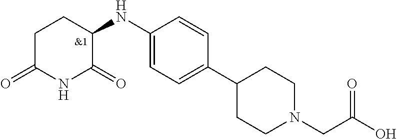 [SMILES: O=C(O)CN1CCC(c2ccc(N[C@@H]3CCC(=O)NC3=O)cc2)CC1]

To a solution of rac-(R)-3-((4-(piperidin-4-yl)phenyl)amino)piperidine-2,6-dione (200.00 mg, 0.6176 mmol) and N,N-diisopropylethylamine (431.49 μL, 0.32 g, 2.4705 mmol) was added bromo acetic acid (96.06 μL, 128.73 mg, 0.9264 mmol) and the reaction mixture was stirred until complete by LCMS. Then, it was concentrated to dryness and used in the next step without further purification. LCMS: C₁₈H₂₃N₃O₄ requires 345.2. found: m/z=346.1 [M+H]⁺.

### Step 3: Synthesis of the Title Compound

The title compound was synthesized according to Step 3 of Example 2 using (1-(6-(2-hydroxyphenyl)pyridazin-4-yl)-4-phenylpiperidin-4-yl)(2,7-diazaspiro[3.5]nonan-2-yl)methanone and rac-(R)-2-(4-(4-((2,6-dioxopiperidin-3-yl)amino)phenyl)piperidin-1-yl)acetic acid. The solution was injected onto RP-FC and purified with a gradient of 5-95% MeCN in H₂O to furnish the title compound (5.6 mg, 16%). LCMS: C₄₇H₅₄N₈O₅ requires 810.4. found: m/z=811.3 [M+H]⁺.

### Example 109

### rac-1-(2-(4-(4-(2,6-dioxopiperidin-3-yl)phenyl)piperidin-1-yl)acetyl)-n-((1-(6-(2-hydroxyphenyl)pyridazin-4-yl)-4-phenylpiperidin-4-yl)methyl)piperidine-4-sulfonamide

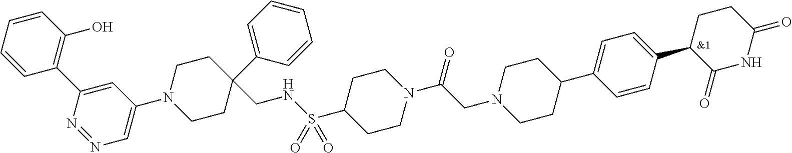 [SMILES: O=C1CC[C@H](c2ccc(C3CCN(CC(=O)N4CCC(S(=O)(=O)NCC5(c6ccccc6)CCN(c6cnnc(-c7ccccc7O)c6)CC5)CC4)CC3)cc2)C(=O)N1]

### Step 1: Synthesis of N-((1-(6-(2-hydroxyphenyl)pyridazin-4-yl)-4-phenylpiperidin-4-yl)methyl)piperidine-4-sulfonamide

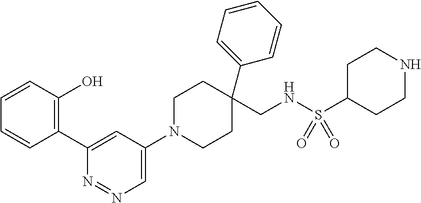 [SMILES: O=S(=O)(NCC1(c2ccccc2)CCN(c2cnnc(-c3ccccc3O)c2)CC1)C1CCNCC1]

2-[5-(4-amino-4-phenylpiperidin-1-yl)pyridazin-3-yl]phenol hydrochloride (30.00 mg, 0.0784 mmol) was dissolved in 1 mL DMA and cooled to 0 C in an ice bath and Triethylamine (0.02 mL, 15.86 mg, 0.1567 mmol) was added in one portion to the solution. Tert-butyl 4-(chlorosulfonyl)piperidine-1-carboxylate (33.35 mg, 0.1175 mmol) was then added to the reaction in one portion. The reaction was warmed to RT and stirred at 80 C overnight. After 12 h, another equivalent of tert-butyl 4-(chlorosulfonyl)piperidine-1-carboxylate (33.35 mg, 0.1175 mmol) was added and the reaction was continued stirring at 80 C for 12 h. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step LCMS: C₂₇H₃₃N₅O₃S requires 507.2. found: m/z=508.3 [M+H]⁺.

### Step 2: Synthesis of the Title Compound

N-((1-(6-(2-hydroxyphenyl)pyridazin-4-yl)-4-phenylpiperidin-4-yl)methyl)piperidine-4-sulfonamide (11.2 mg, 0.022 mmol) and rac-2-(4-(4-(2,6-dioxopiperidin-3-yl)phenyl)piperidin-1-yl)acetic acid (7.31 mg, 0.022 mmol) (synthesized as described in WO2022032026 A1 2022-02-10) were dissolved in 0.5 mL DMA. N,N-diisopropylethylamine (14.3 mg, 0.110 mmol) followed by addition of [(dimethylamino)({[1,2,3]triazolo[4,5-b]pyridin-3-yloxy})methylidene]dimethylazanium; hexafluoro-lambda5-phosphanuide (12.6 mg, 0.033 mmol) in one portion and the resulting solution was stirred at room temperature for 1 h. The reaction was directly injected onto and purified with a gradient of 0-80% MeCN in H₂O to yield the title compound (1.1 mg, 6.1%). LCMS: C₄₅H₅₃N₇O₆S requires 819.3. found: m/z=820.4 [M+H]⁺.

### Example 110

### rac-n-(1-(2-(4-(4-(2,6-dioxopiperidin-3-yl)phenyl)piperidin-1-yl)acetyl)piperidin-4-yl)-1-(6-(2-hydroxyphenyl)pyridazin-4-yl)-n-methyl-4-((1-methyl-1h-pyrazol-3-yl)oxy)piperidine-4-carboxamide

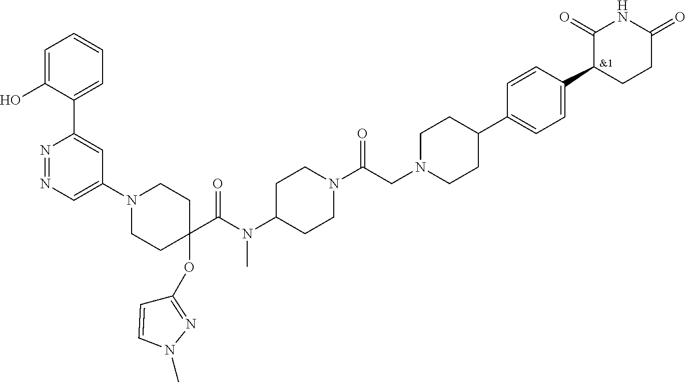 [SMILES: CN(C(=O)C1(Oc2ccn(C)n2)CCN(c2cnnc(-c3ccccc3O)c2)CC1)C1CCN(C(=O)CN2CCC(c3ccc([C@@H]4CCC(=O)NC4=O)cc3)CC2)CC1]

### Step 1: Synthesis of 1-(6-(2-hydroxyphenyl)pyridazin-4-yl)-N-methyl-4-((1-methyl-1H-pyrazol-3-yl)oxy)-N-(piperidin-4-yl)piperidine-4-carboxamide

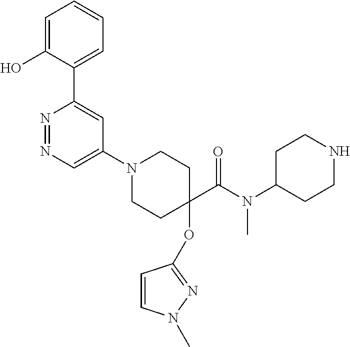 [SMILES: CN(C(=O)C1(Oc2ccn(C)n2)CCN(c2cnnc(-c3ccccc3O)c2)CC1)C1CCNCC1]

1-(6-(2-hydroxyphenyl)pyridazin-4-yl)-4-((1-methyl-1H-pyrazol-3-yl)oxy)piperidine-4-carboxylic acid (55.63 mg, 0.1407 mmol) and tert-butyl 4-(methylamino)piperidine-1-carboxylate (0.03 mL, 30.15 mg, 0.1407 mmol) were dissolved in 1.0 mL DMA. N,N-diisopropylethylamine (0.07 mL, 54.55 mg, 0.4221 mmol) followed by addition of [(dimethylamino)({[1,2,3]triazolo[4,5-b]pyridin-3-yloxy})methylidene]dimethylazanium; hexafluoro-lambda5-phosphanuide (80.24 mg, 0.2110 mmol in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂₆H₃₃N₇O₃ requires 491.2. found: m/z=492.4 [M+H]⁺.

### Step 2: Synthesis of the Title Compound

1-(6-(2-hydroxyphenyl)pyridazin-4-yl)-N-methyl-4-((1-methyl-1H-pyrazol-3-yl)oxy)-N-(piperidin-4-yl)piperidine-4-carboxamide (10.8 mg, 0.022 mmol) and rac-2-(4-(4-(2,6-dioxopiperidin-3-yl)phenyl)piperidin-1-yl)acetic acid (7.31 mg, 0.022 mmol) were dissolved in 0.5 mL DMA. N,N-diisopropylethylamine (14.3 mg, 0.110 mmol) followed by addition of [(dimethylamino)({[1,2,3]triazolo[4,5-b]pyridin-3-yloxy})methylidene]dimethylazanium; hexafluoro-lambda5-phosphanuide (12.6 mg, 0.033 mmol) in one portion and the resulting solution was stirred at room temperature for 1 h. The reaction was directly injected onto RP-FC and purified with a gradient of 0-80% MeCN in H₂O to yield the title compound (5.1, 28.9%). LCMS: C₄₄H₅₃N₉O₆ requires 803.4. found: m/z=804.4 [M+H]⁺.

### Example 111

### rac-n-(1-(2-(4-(4-(2,6-dioxopiperidin-3-yl)phenyl)piperidin-1-yl)acetyl)piperidin-4-yl)-1-(6-(2-hydroxyphenyl)pyridazin-4-yl)-4-methoxy-n-methylpiperidine-4-carboxamide

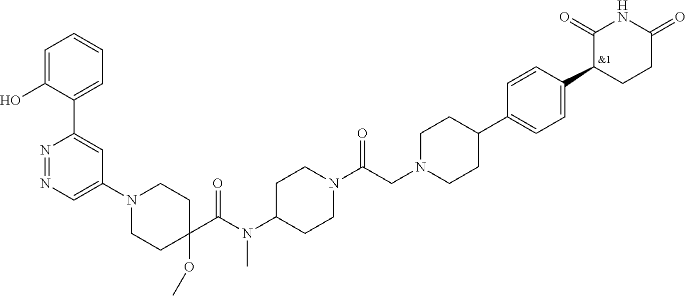 [SMILES: COC1(C(=O)N(C)C2CCN(C(=O)CN3CCC(c4ccc([C@@H]5CCC(=O)NC5=O)cc4)CC3)CC2)CCN(c2cnnc(-c3ccccc3O)c2)CC1]

### Step 1: Synthesis of the Title Compound

1-(6-(2-hydroxyphenyl)pyridazin-4-yl)-4-methoxy-N-methyl-N-(piperidin-4-yl)piperidine-4-carboxamide (9.36, 0.022 mmol) and rac-2-(4-(4-(2,6-dioxopiperidin-3-yl)phenyl)piperidin-1-yl)acetic acid (7.31 mg, 0.022 mmol) were dissolved in 0.5 mL DMA. N,N-diisopropylethylamine (14.3 mg, 0.110 mmol) was followed by addition of [(dimethylamino)({[1,2,3]triazolo[4,5-b]pyridin-3-yloxy})methylidene]dimethylazanium; hexafluoro-lambda5-phosphanuide (12.6 mg, 0.033 mmol) in one portion and the resulting solution was stirred at room temperature for 1 h. The reaction was directly injected onto RP-FC and purified with a gradient of 0-80% MeCN in H₂O to yield the title compound (5.1 mg, 31.4%). LCMS: C₄₁H₅₁N₇O₆ requires 737.4. found: m/z=738.3 [M+H]⁺.

### Example 112

### rac-n-(1-(2-(4-(4-(2,6-dioxopiperidin-3-yl)phenyl)piperidin-1-yl)acetyl)piperidin-4-yl)-1-(6-(2-hydroxyphenyl)pyridazin-4-yl)-n-methyl-4-((1-methyl-1h-pyrazol-3-yl)oxy)piperidine-4-carboxamide

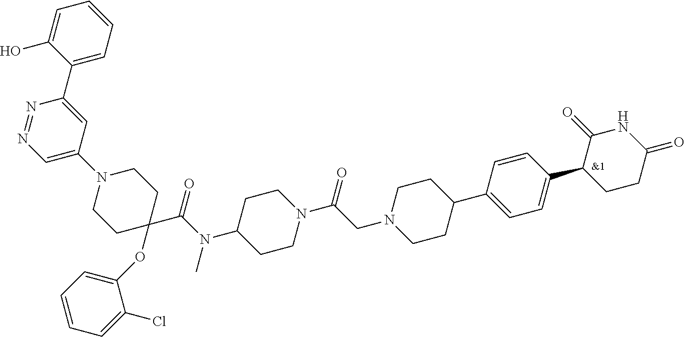 [SMILES: CN(C(=O)C1(Oc2ccccc2Cl)CCN(c2cnnc(-c3ccccc3O)c2)CC1)C1CCN(C(=O)CN2CCC(c3ccc([C@@H]4CCC(=O)NC4=O)cc3)CC2)CC1]

### Step 1: Synthesis of 4-(2-chlorophenoxy)-1-(6-(2-hydroxyphenyl)pyridazin-4-yl)-N-methyl-N-(piperidin-4-yl)piperidine-4-carboxamide

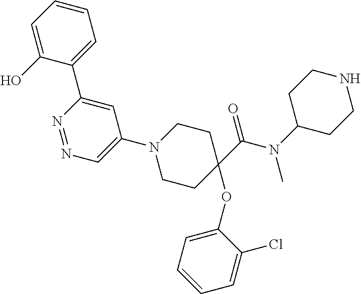 [SMILES: CN(C(=O)C1(Oc2ccccc2Cl)CCN(c2cnnc(-c3ccccc3O)c2)CC1)C1CCNCC1]

4-(2-chlorophenoxy)-1-(6-(2-hydroxyphenyl)pyridazin-4-yl)piperidine-4-carboxylic acid (Intermediate 25) (59.91 mg, 0.1407 mmol) and tert-butyl 4-(methylamino)piperidine-1-carboxylate (0.03 mL, 30.15 mg, 0.1407 mmol) were dissolved in 1.0 mL DMA. N,N-diisopropylethylamine (0.07 mL, 54.6 mg, 422 mmol) followed by addition of [(dimethylamino)({[1,2,3]triazolo[4,5-b]pyridin-3-yloxy})methylidene]dimethylazanium; hexafluoro-lambda5-phosphanuide (80.2 mg, 211 mmol in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂8H₃₂ClN₅O₃ requires 521.2. found: m/z=522.3 [M+H]⁺.

### Step 2: Synthesis of the Title Compound

4-(2-chlorophenoxy)-1-(6-(2-hydroxyphenyl)pyridazin-4-yl)-N-methyl-N-(piperidin-4-yl)piperidine-4-carboxamide (11.5 mg, 0.022 mmol) and rac-2-(4-(4-(2,6-dioxopiperidin-3-yl)phenyl)piperidin-1-yl)acetic acid (7.31 mg, 0.022 mmol) were dissolved in 0.5 mL DMA. N,N-diisopropylethylamine (14.3 mg, 0.110 mmol) was added followed by addition of [(dimethylamino)({[1,2,3]triazolo[4,5-b]pyridin-3-yloxy})methylidene]dimethylazanium; hexafluoro-lambda5-phosphanuide (12.6 mg, 0.033 mmol) in one portion and the resulting solution was stirred at room temperature for 1 h. The reaction was directly injected onto RP-FC and purified with a gradient of 0-80% MeCN in H₂O to yield the title compound (4.6, 25.1%). LCMS: C₄₆H₅₂ClN₇O₆ requires 833.4. found: m/z=834.4 [M+H]⁺.

### Example 113

### rac-(r)-3-(4-(1-(2-(6-(4-(2-chlorophenoxy)-1-(6-(2-hydroxyphenyl)pyridazin-4-yl)piperidine-4-carbonyl)-2,6-diazaspiro[3.3]heptan-2-yl)-2-oxoethyl)piperidin-4-yl)phenyl)piperidine-2,6-dione

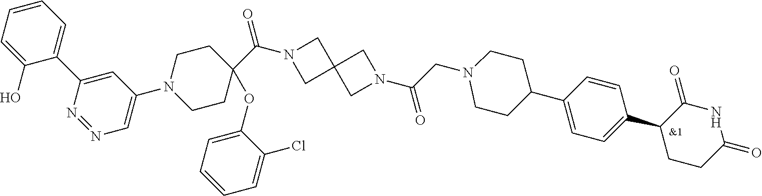 [SMILES: O=C1CC[C@@H](c2ccc(C3CCN(CC(=O)N4CC5(C4)CN(C(=O)C4(Oc6ccccc6Cl)CCN(c6cnnc(-c7ccccc7O)c6)CC4)C5)CC3)cc2)C(=O)N1]

### Step 1: Synthesis of (4-(2-chlorophenoxy)-1-(6-(2-hydroxyphenyl)pyridazin-4-yl)piperidin-4-yl)(2,6-diazaspiro[3.3]heptan-2-yl)methanone

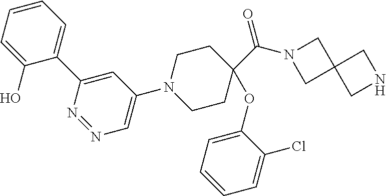 [SMILES: O=C(N1CC2(CNC2)C1)C1(Oc2ccccc2Cl)CCN(c2cnnc(-c3ccccc3O)c2)CC1]

4-(2-chlorophenoxy)-1-(6-(2-hydroxyphenyl)pyridazin-4-yl)piperidine-4-carboxylic acid (59.91 mg, 0.1407 mmol) and tert-butyl 2,6-diazaspiro[3.3]heptane-2-carboxylate (27.9 mg, 141 mmol) were dissolved in 1.0 mL DMA. N,N-diisopropylethylamine (0.07 mL, 54.55 mg, 0.4221 mmol) followed by addition of [(dimethylamino)({[1,2,3]triazolo[4,5-b]pyridin-3-yloxy})methylidene]dimethylazanium; hexafluoro-lambda5-phosphanuide (80.2 mg, 211 mmol in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂₇H₂8ClN₅O₃ requires 505.1. found: m/z=506.4 [M+H]⁺.

### Step 2: Synthesis of the Title Compound

(4-(2-chlorophenoxy)-1-(6-(2-hydroxyphenyl)pyridazin-4-yl)piperidin-4-yl)(2,6-diazaspiro[3.3]heptan-2-yl)methanone (11.1 mg, 0.022 mmol) and rac-2-(4-(4-(2,6-dioxopiperidin-3-yl)phenyl)piperidin-1-yl)acetic acid (7.31 mg, 0.022 mmol) were dissolved in 0.5 mL DMA. N,N-diisopropylethylamine (14.3 mg, 0.110 mmol) was added followed by addition of [(dimethylamino)({[1,2,3]triazolo[4,5-b]pyridin-3-yloxy})methylidene]dimethylazanium; hexafluoro-lambda5-phosphanuide (12.6 mg, 0.033 mmol) in one portion and the resulting solution was stirred at room temperature for 1 h. The reaction was directly injected onto RP-FC and purified with a gradient of 0-80% MeCN in H₂O to yield the title compound (4.7 mg, 26.1%). LCMS: C₄₅H₄₈ClN₇O₆ requires 817.3. found: m/z=818.2 [M+H]⁺.

### Example 114

### (r)-4-cyclopropoxy-n-(1-((1-(4-(2,6-dioxopiperidin-3-yl)phenyl)piperidin-4-yl)methyl)piperidin-4-yl)-1-(6-(2-hydroxyphenyl)pyridazin-4-yl)-n-methylpiperidine-4-carboxamide

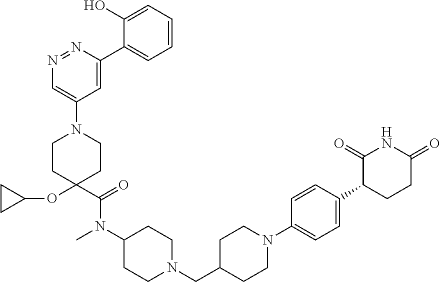 [SMILES: CN(C(=O)C1(OC2CC2)CCN(c2cnnc(-c3ccccc3O)c2)CC1)C1CCN(CC2CCN(c3ccc([C@H]4CCC(=O)NC4=O)cc3)CC2)CC1]

### Step 1: Synthesis of the Title Compound

4-cyclopropoxy-1-(6-(2-hydroxyphenyl)pyridazin-4-yl)-N-methyl-N-(piperidin-4-yl)piperidine-4-carboxamide (10.0 mg, 0.022 mmol) and (R)-1-(4-(2,6-dioxopiperidin-3-yl)phenyl)piperidine-4-carbaldehyde (6.60 mg, 0.022 mmol) were dissolved in 0.5 mL DMA. N,N-diisopropylethylamine (14.3 mg, 0.110 mmol) followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (7.0 mg, 43.3%). LCMS: C₄₂H₅₃N₇O₅ requires 735.4. found: m/z=736.4 [M+H]⁺.

### Example 115

### rac-4-cyclopropoxy-n-(1-(2-(4-(4-(2,6-dioxopiperidin-3-yl)phenyl)piperidin-1-yl)acetyl)piperidin-4-yl)-1-(6-(2-hydroxyphenyl)pyridazin-4-yl)-n-methylpiperidine-4-carboxamide

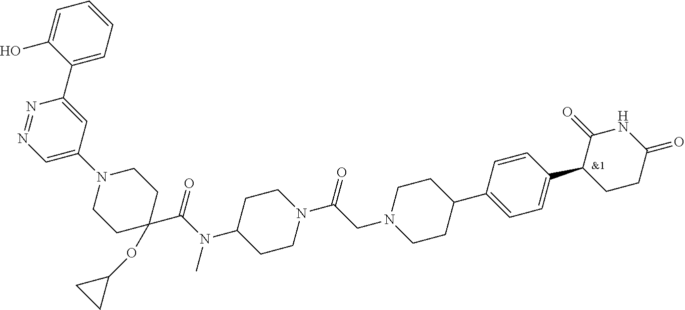 [SMILES: CN(C(=O)C1(OC2CC2)CCN(c2cnnc(-c3ccccc3O)c2)CC1)C1CCN(C(=O)CN2CCC(c3ccc([C@@H]4CCC(=O)NC4=O)cc3)CC2)CC1]

### Step 1: Synthesis of the Title Compound

4-cyclopropoxy-1-(6-(2-hydroxyphenyl)pyridazin-4-yl)-N-methyl-N-(piperidin-4-yl)piperidine-4-carboxamide (10.0 mg, 0.022 mmol) and rac-2-(4-(4-(2,6-dioxopiperidin-3-yl)phenyl)piperidin-1-yl)acetic acid (7.31 mg, 0.022 mmol) were dissolved in 0.5 mL DMA. N,N-diisopropylethylamine (14.3 mg, 0.110 mmol) followed by addition of [(dimethylamino)({[1,2,3]triazolo[4,5-b]pyridin-3-yloxy})methylidene]dimethylazanium; hexafluoro-lambda5-phosphanuide (12.6 mg, 0.033 mmol) in one portion and the resulting solution was stirred at room temperature for 1 h. The reaction was directly injected onto RP-FC and purified with a gradient of 0-80% MeCN in H2O to yield the title compound (4.00 mg, 23.6%). LCMS: C₄₃H₅₃N₇O₆ requires 763.4. found: m/z=764.4 [M+H]⁺.

### Example 116

### rac-n-(1-(2-(4-(4-(2,6-dioxopiperidin-3-yl)phenyl)piperidin-1-yl)acetyl)piperidin-4-yl)-1-(6-(2-hydroxyphenyl)pyridazin-4-yl)-n-methyl-4-(o-tolyloxy)piperidine-4-carboxamide

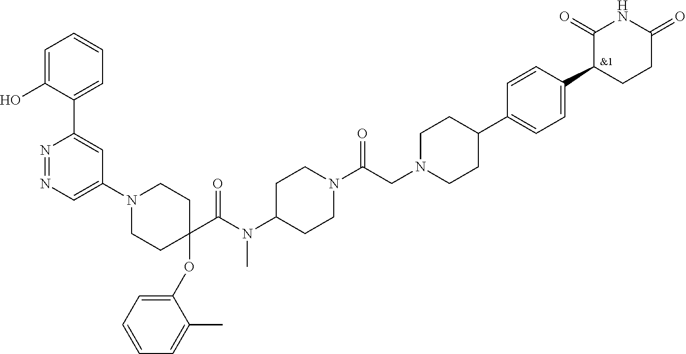 [SMILES: Cc1ccccc1OC1(C(=O)N(C)C2CCN(C(=O)CN3CCC(c4ccc([C@@H]5CCC(=O)NC5=O)cc4)CC3)CC2)CCN(c2cnnc(-c3ccccc3O)c2)CC1]

### Step 1: Synthesis of 1-(6-(2-hydroxyphenyl)pyridazin-4-yl)-N-methyl-N-(piperidin-4-yl)-4-(o-tolyloxy)piperidine-4-carboxamide

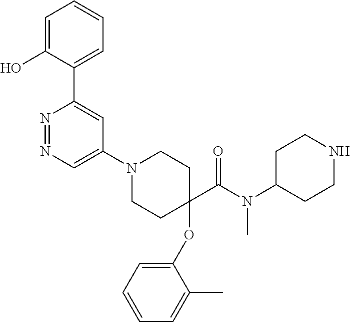 [SMILES: Cc1ccccc1OC1(C(=O)N(C)C2CCNCC2)CCN(c2cnnc(-c3ccccc3O)c2)CC1]

1-(6-(2-hydroxyphenyl)pyridazin-4-yl)-4-(o-tolyloxy)piperidine-4-carboxylic acid (Intermediate 16) (57.04 mg, 0.1407 mmol) and tert-butyl 4-(methylamino)piperidine-1-carboxylate (0.03 mL, 30.15 mg, 0.1407 mmol) were dissolved in 1.0 mL DMA. N,N-diisopropylethylamine (0.07 mL, 54.55 mg, 0.4221 mmol) was added followed by addition of [(dimethylamino)({[1,2,3]triazolo[4,5-b]pyridin-3-yloxy})methylidene]dimethylazanium; hexafluoro-lambda5-phosphanuide (80.24 mg, 0.2110 mmol in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂₉H₃₅N₅O₃ requires 501.2. found: m/z=502.3 [M+H]⁺.

### Step 2: Synthesis of the Title Compound

1-(6-(2-hydroxyphenyl)pyridazin-4-yl)-N-methyl-N-(piperidin-4-yl)-4-(o-tolyloxy)piperidine-4-carboxamide (11.0 mg, 0.022 mmol) and rac-2-(4-(4-(2,6-dioxopiperidin-3-yl)phenyl)piperidin-1-yl)acetic acid (7.31 mg, 0.022 mmol) were dissolved in 0.5 mL DMA. N,N-diisopropylethylamine (14.3 mg, 0.110 mmol) was added followed by addition of [(dimethylamino)({[1,2,3]triazolo[4,5-b]pyridin-3-yloxy})methylidene]dimethylazanium; hexafluoro-lambda5-phosphanuide (12.6 mg, 0.033 mmol) in one portion and the resulting solution was stirred at room temperature for 1 h. The reaction was directly injected onto RP-FC and purified with a gradient of 0-80% MeCN in H₂O to yield the title compound (3.9 mg, 21.8%). LCMS: C₄₇H₅₅N₇O₆ requires 813.4. found: m/z=814.4 [M+H]⁺.

### Example 117

### rac-4-(cyclohexyloxy)-n-(1-(2-(4-(4-(2,6-dioxopiperidin-3-yl)phenyl)piperidin-1-yl)acetyl)piperidin-4-yl)-1-(6-(2-hydroxyphenyl)pyridazin-4-yl)-n-methylpiperidine-4-carboxamide

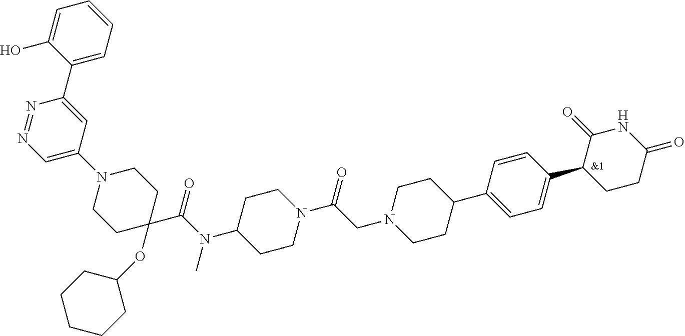 [SMILES: CN(C(=O)C1(OC2CCCCC2)CCN(c2cnnc(-c3ccccc3O)c2)CC1)C1CCN(C(=O)CN2CCC(c3ccc([C@@H]4CCC(=O)NC4=O)cc3)CC2)CC1]

### Step 1: Synthesis of 4-(cyclohexyloxy)-1-(6-(2-hydroxyphenyl)pyridazin-4-yl)-N-methyl-N-(piperidin-4-yl)piperidine-4-carboxamide

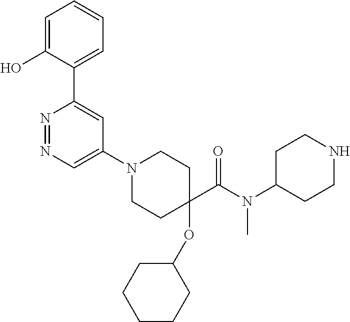 [SMILES: CN(C(=O)C1(OC2CCCCC2)CCN(c2cnnc(-c3ccccc3O)c2)CC1)C1CCNCC1]

4-(cyclohexyloxy)-1-(6-(2-hydroxyphenyl)pyridazin-4-yl)piperidine-4-carboxylic acid (Intermediate 23) (55.92 mg, 0.1407 mmol) and tert-butyl 4-(methylamino)piperidine-1-carboxylate (0.03 mL, 30.15 mg, 0.141 mmol) were dissolved in 1.0 mL DMA. N,N-diisopropylethylamine (0.07 mL, 54.55 mg, 0.422 mmol) was added followed by addition of [(dimethylamino)({[1,2,3]triazolo[4,5-b]pyridin-3-yloxy})methylidene]dimethylazanium; hexafluoro-lambda5-phosphanuide (80.24 mg, 0.2110 mmol in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂₈H₃₉N₅O₃ requires 493.3. found: m/z=494.3 [M+H]⁺.

### Step 2: Synthesis of the Title Compound

4-(cyclohexyloxy)-1-(6-(2-hydroxyphenyl)pyridazin-4-yl)-N-methyl-N-(piperidin-4-yl)piperidine-4-carboxamide (10.8 mg, 0.022 mmol) and rac-2-(4-(4-(2,6-dioxopiperidin-3-yl)phenyl)piperidin-1-yl)acetic acid (7.31 mg, 0.022 mmol) were dissolved in 0.5 mL DMA. N,N-diisopropylethylamine (14.3 mg, 0.110 mmol) was added followed by addition of [(dimethylamino)({[1,2,3]triazolo[4,5-b]pyridin-3-yloxy})methylidene]dimethylazanium; hexafluoro-lambda5-phosphanuide (12.6 mg, 0.033 mmol) in one portion and the resulting solution was stirred at room temperature for 1 h. The reaction was directly injected onto RP-FC and purified with a gradient of 0-80% MeCN in H₂O to yield the title compound (3.1 mg, 17.5%). LCMS: C₄₆H₅₉N₇O₅ requires 805.5. found: m/z=806.4 [M+H]⁺.

### Example 118

### rac-1-(2-(1-(4-(2,6-dioxopiperidin-3-yl)phenyl)piperidin-4-yl)ethyl)-n-((1-(6-(2-hydroxyphenyl)pyridazin-4-yl)-4-phenylpiperidin-4-yl)methyl)piperidine-4-sulfonamide

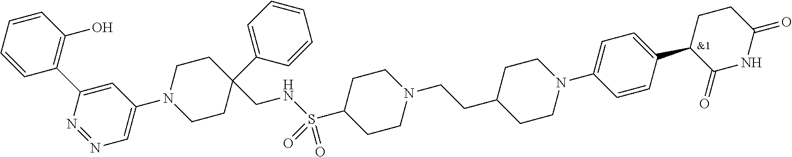 [SMILES: O=C1CC[C@H](c2ccc(N3CCC(CCN4CCC(S(=O)(=O)NCC5(c6ccccc6)CCN(c6cnnc(-c7ccccc7O)c6)CC5)CC4)CC3)cc2)C(=O)N1]

### Step 1: Synthesis of the Title Compound

N-((1-(6-(2-hydroxyphenyl)pyridazin-4-yl)-4-phenylpiperidin-4-yl)methyl)piperidine-4-sulfonamide (11.2 mg, 0.022 mmol) and rac-1-(4-(2,6-dioxopiperidin-3-yl)phenyl)piperidine-4-carbaldehyde (6.91 mg, 0.022 mmol) were dissolved in 0.5 mL DMA. N,N-diisopropylethylamine (14.3 mg, 0.110 mmol) was added, followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (3.0 mg, 16.9%). LCMS: C₄₅H₅₅N₇O₅S requires 805.4. found: m/z=806.6 [M+H]⁺.

### Example 119

### (r)-3-(4-(4-((6-(4-(2-chlorophenoxy)-1-(6-(2-hydroxyphenyl)pyridazin-4-yl)piperidine-4-carbonyl)-2,6-diazaspiro[3.3]heptan-2-yl)methyl)piperidin-1-yl)phenyl)piperidine-2,6-dione

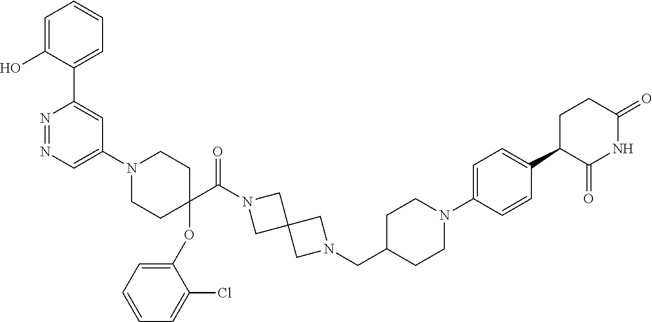 [SMILES: O=C1CC[C@H](c2ccc(N3CCC(CN4CC5(C4)CN(C(=O)C4(Oc6ccccc6Cl)CCN(c6cnnc(-c7ccccc7O)c6)CC4)C5)CC3)cc2)C(=O)N1]

### Step 1: Synthesis of the Title Compound

(4-(2-chlorophenoxy)-1-(6-(2-hydroxyphenyl)pyridazin-4-yl)piperidin-4-yl)(2,6-diazaspiro[3.3]heptan-2-yl)methanone (11.1 mg, 0.022 mmol) and (R)-1-(4-(2,6-dioxopiperidin-3-yl)phenyl)piperidine-4-carbaldehyde (6.60 mg, 0.022 mmol) were dissolved in 0.5 mL DMA. N,N-diisopropylethylamine (14.3 mg, 0.110 mmol) was added, followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (5.5 mg, 31.7%). LCMS: C₄₄H₄₈ClN₇O₅ requires 789.3. found: m/z=790.3 [M+H]⁺.

### Example 120

### (r)-3-(4-(4-((7-(1-(6-(2-hydroxyphenyl)pyridazin-4-yl)-4-methoxypiperidine-4-carbonyl)-2,7-diazaspiro[3.5]nonan-2-yl)methyl)piperidin-1-yl)phenyl)piperidine-2,6-dione

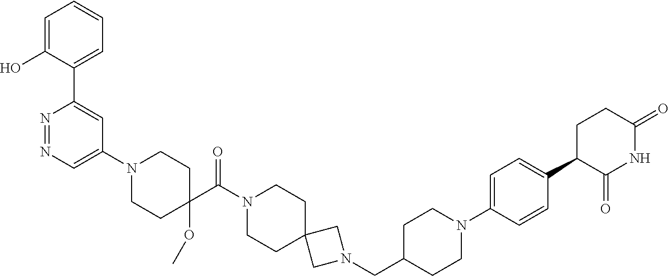 [SMILES: COC1(C(=O)N2CCC3(CC2)CN(CC2CCN(c4ccc([C@H]5CCC(=O)NC5=O)cc4)CC2)C3)CCN(c2cnnc(-c3ccccc3O)c2)CC1]

### Step 1: Synthesis of 1-(6-(2-hydroxyphenyl)pyridazin-4-yl)-4-methoxy-N-methyl-N-(piperidin-4-yl)piperidine-4-carboxamide

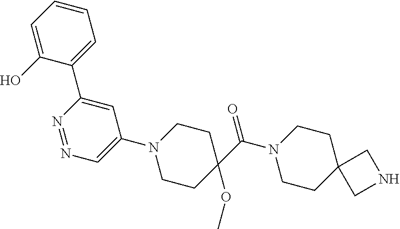 [SMILES: COC1(C(=O)N2CCC3(CC2)CNC3)CCN(c2cnnc(-c3ccccc3O)c2)CC1]

1-(6-(2-hydroxyphenyl)pyridazin-4-yl)-4-methoxypiperidine-4-carboxylic acid (Intermediate 24) (46.30 mg, 0.1407 mmol) and tert-butyl 2,7-diazaspiro[3.5]nonane-2-carboxylate (31.84 mg, 0.1407 mmol) were dissolved in 1.0 mL DMA. N,N-diisopropylethylamine (0.07 mL, 54.55 mg, 0.4221 mmol) and added to a half dram vial, followed by addition of [(dimethylamino)({[1,2,3]triazolo[4,5-b]pyridin-3-yloxy})methylidene]dimethylazanium; hexafluoro-lambda5-phosphanuide (80.24 mg, 0.2110 mmol in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂₄H₃₁N₅O₃ requires 437.2. found: m/z=438.2 [M+H]⁺.

### Step 2: Synthesis of the Title Compound

1-(6-(2-hydroxyphenyl)pyridazin-4-yl)-4-methoxy-N-methyl-N-(piperidin-4-yl)piperidine-4-carboxamide (9.62 mg, 0.022 mmol) and (R)-1-(4-(2,6-dioxopiperidin-3-yl)phenyl)piperidine-4-carbaldehyde (6.60 mg, 0.022 mmol) were dissolved in 0.5 mL DMA. N,N-diisopropylethylamine (14.3 mg, 0.110 mmol), followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (8.4 mg, 52.9%). LCMS: C₄₁H₅₁N₇O₅ requires 721.4, found: m/z=722.3 [M+H]⁺.

### Example 121

### rac-3-(4-(4-(2-(6-(4-(cyclohexyloxy)-1-(6-(2-hydroxyphenyl)pyridazin-4-yl)piperidine-4-carbonyl)-2,6-diazaspiro[3.3]heptan-2-yl)ethyl)piperidin-1-yl)phenyl)piperidine-2,6-dione

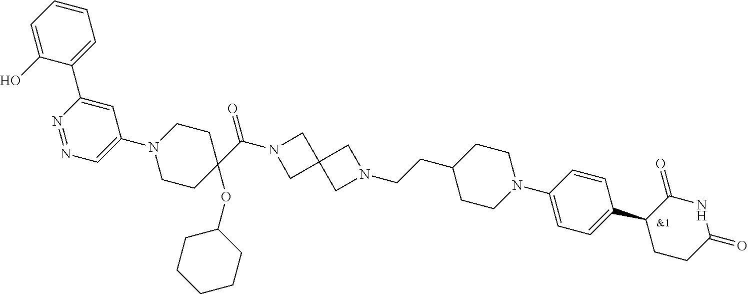 [SMILES: O=C1CC[C@@H](c2ccc(N3CCC(CCN4CC5(C4)CN(C(=O)C4(OC6CCCCC6)CCN(c6cnnc(-c7ccccc7O)c6)CC4)C5)CC3)cc2)C(=O)N1]

### Step 1: Synthesis of (4-(cyclohexyloxy)-1-(6-(2-hydroxyphenyl)pyridazin-4-yl)piperidin-4-yl)(2,6-diazaspiro[3.3]heptan-2-yl)methanone

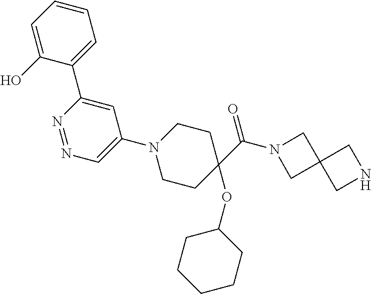 [SMILES: O=C(N1CC2(CNC2)C1)C1(OC2CCCCC2)CCN(c2cnnc(-c3ccccc3O)c2)CC1]

4-(cyclohexyloxy)-1-(6-(2-hydroxyphenyl)pyridazin-4-yl)piperidine-4-carboxylic acid (55.92 mg, 0.1407 mmol) and tert-butyl 2,6-diazaspiro[3.3]heptane-2-carboxylate (27.89 mg, 0.1407 mmol) were dissolved in 1.0 mL DMA. N,N-diisopropylethylamine (0.07 mL, 54.55 mg, 0.4221 mmol), followed by addition of [(dimethylamino)({[1,2,3]triazolo[4,5-b]pyridin-3-yloxy})methylidene]dimethylazanium; hexafluoro-lambda5-phosphanuide (80.24 mg, 0.2110 mmol in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step (please add compound details). LCMS: C₂₇H₃₅N₅O₃ requires 477.2. found: m/z=478.3 [M+H]⁺.

### Step 2: Synthesis of the Title Compound

(4-(cyclohexyloxy)-1-(6-(2-hydroxyphenyl)pyridazin-4-yl)piperidin-4-yl)(2,6-diazaspiro[3.3]heptan-2-yl)methanone (10.5 mg, 0.022 mmol) and rac-1-(4-(2,6-dioxopiperidin-3-yl)phenyl)piperidine-4-carbaldehyde (6.91 mg, 0.022 mmol) were dissolved in 0.5 mL DMA. N,N-diisopropylethylamine (14.3 mg, 0.110 mmol), followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (10.8 mg, 63.3%). LCMS: C₄₅H₅₇N₇O₅ requires 775.4. found: m/z=776.3 [M+H]⁺.

### Example 122

### rac-(3R)-3-(4-{4-fluoro-4-[2-(6-{1-[6-(2-hydroxyphenyl)pyridazin-4-yl]-4-phenylpiperidine-4-carbonyl}-2,6-diazaspiro[3.3]heptan-2-yl)ethyl]piperidin-1-yl}phenyl)piperidine-2,6-dione

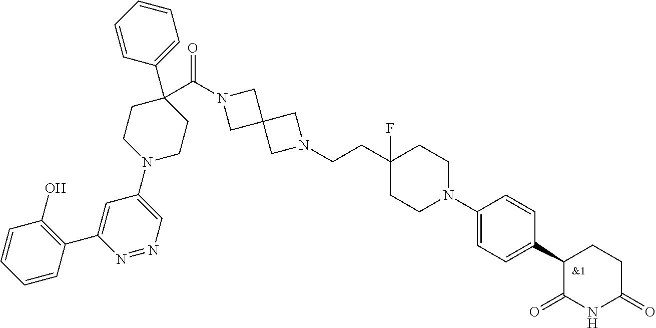 [SMILES: O=C1CC[C@H](c2ccc(N3CCC(F)(CCN4CC5(C4)CN(C(=O)C4(c6ccccc6)CCN(c6cnnc(-c7ccccc7O)c6)CC4)C5)CC3)cc2)C(=O)N1]

Combined 2-[5-(4-{2,6-diazaspiro[3.3]heptane-2-carbonyl}-4-phenylpiperidin-1-yl)pyridazin-3-yl]phenol; trifluoroacetic acid (22.00 mg, 0.0386 mmol) and rac-2-(1-{4-[(3R)-2,6-dioxopiperidin-3-yl]phenyl}-4-fluoropiperidin-4-yl)acetaldehyde (14.12 mg, 0.0425 mmol) in DMA (0.20 mL) and then added sodium triacetoxyborohydride (24.56 mg, 0.1159 mmol) and stirred for 2 h. Purified the solution directly by RP-HPLC to yield the title compound (0.0079 g, 26.23%). LCMS: C₄₅H₅₀FN₇O₄ requires: 771.4. found: m/z=772.3 [M+H]⁺.

### Example 123

### 1-(7-{4-[(6-{1-[6-(2-hydroxyphenyl)pyridazin-4-yl]-4-phenylpiperidine-4-carbonyl}-2,6-diazaspiro[3.3]heptan-2-yl)methyl]piperidin-1-yl}-1-methyl-1H-indazol-3-yl)-1,3-diazinane-2,4-dione

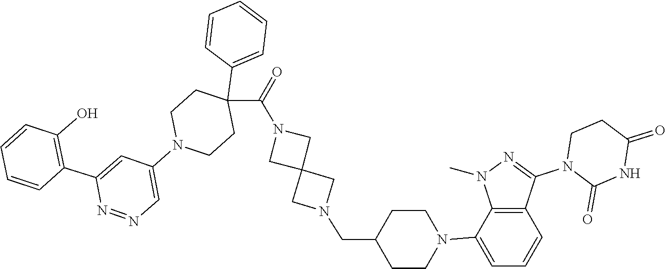 [SMILES: Cn1nc(N2CCC(=O)NC2=O)c2cccc(N3CCC(CN4CC5(C4)CN(C(=O)C4(c6ccccc6)CCN(c6cnnc(-c7ccccc7O)c6)CC4)C5)CC3)c21]

Combined 2-[5-(4-{2,6-diazaspiro[3.3]heptane-2-carbonyl}-4-phenylpiperidin-1-yl)pyridazin-3-yl]phenol; trifluoroacetic acid (22.00 mg, 0.0376 mmol) and 1-[3-(2,4-dioxo-1,3-diazinan-1-yl)-1-methylindazol-7-yl]piperidine-4-carbaldehyde (13.73 mg, 0.0413 mmol) Synthesis as described in WO2023249970 A1 2023-12-28 in DMA (0.20 mL) with N,N-diisopropylethylamine (32.72 μL, 0.02 g, 0.1878 mmol) and then added sodium triacetoxyborohydride (23.89 mg, 0.1127 mmol) and stirred for 2 h. Purified the solution directly by RP-HPLC to yield the title compound (0.0125 g, 41.10%). LCMS: C₄₅H₅₀N₁₀O₄ requires: 794.4. found: m/z=795.3 [M+H]^{+ 1}H NMR (500 MHz, DMSO) δ 10.54 (s, 1H), 8.96 (d, J=2.9 Hz, 1H), 8.14 (s, 1H), 8.10 (d, J=8.1 Hz, 1H), 7.55 (d, J=2.9 Hz, 1H), 7.42 (t, J=7.6 Hz, 2H), 7.34 (d, J=7.9 Hz, 4H), 7.28 (d, J=4.8 Hz, 1H), 7.00 (d, J=5.4 Hz, 2H), 6.96-6.90 (m, 2H), 4.20 (s, 3H), 4.06 (d, J=13.6 Hz, 2H), 3.97 (s, 1H), 3.87 (t, J=6.7 Hz, 2H), 3.63 (s, 1H), 3.42-3.35 (m, 4H), 3.29 (d, J=8.2 Hz, 4H), 3.21 (s, 2H), 2.76 (t, J=6.6 Hz, 2H), 1.97 (s, 1H), 1.73 (s, 1H), 1.35 (s, 1H).

### Example 124

### rac-4-cyclopropoxy-N-{1-[2-(1-{4-[(3r)-2,6-dioxopiperidin-3-yl]phenyl}piperidin-4-yl)ethyl]piperidin-4-yl}-1-[6-(2-hydroxyphenyl)pyridazin-4-yl]-N-methylpiperidine-4-carboxamide

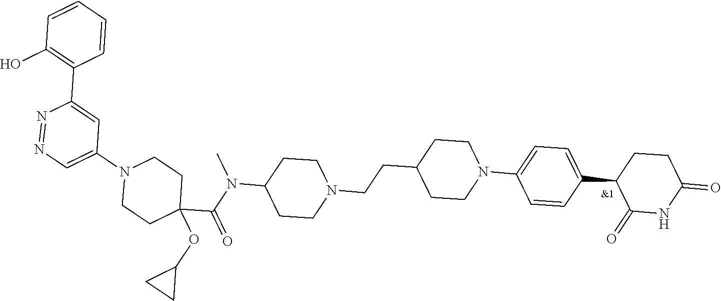 [SMILES: CN(C(=O)C1(OC2CC2)CCN(c2cnnc(-c3ccccc3O)c2)CC1)C1CCN(CCC2CCN(c3ccc([C@H]4CCC(=O)NC4=O)cc3)CC2)CC1]

LCMS: C₄₃H₅₅N₇O₅ requires: 749.4. found: m/z=750.4 [M+H]⁺

### Example 125

### (r)-n-(1-((1-(4-(2,6-dioxopiperidin-3-yl)phenyl)piperidin-4-yl)methyl)piperidin-4-yl)-1-(6-(2-hydroxyphenyl)pyridazin-4-yl)-n-methyl-4-((1-methyl-1h-pyrazol-3-yl)oxy)piperidine-4-carboxamide

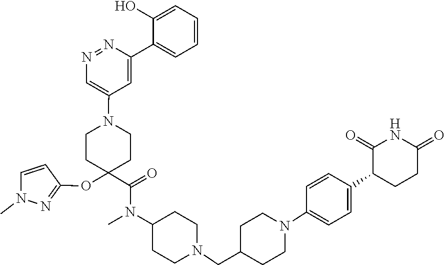 [SMILES: CN(C(=O)C1(Oc2ccn(C)n2)CCN(c2cnnc(-c3ccccc3O)c2)CC1)C1CCN(CC2CCN(c3ccc([C@H]4CCC(=O)NC4=O)cc3)CC2)CC1]

### Step 1: Synthesis of the Title Compound

1-(6-(2-hydroxyphenyl)pyridazin-4-yl)-N-methyl-4-((1-methyl-1H-pyrazol-3-yl)oxy)-N-(piperidin-4-yl)piperidine-4-carboxamide (10.8 mg, 0.022 mmol) and (R)-1-(4-(2,6-dioxopiperidin-3-yl)phenyl)piperidine-4-carbaldehyde (6.60 mg, 0.022 mmol) were dissolved in 0.5 mL DMA. N,N-diisopropylethylamine (14.3 mg, 0.110 mmol), followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (8.8 mg, 51.6%). LCMS: C₄₃H₅₃N₉O₅ requires 775.4. found: m/z=776.3 [M+H]⁺.

### Example 126

### rac-(3R)-3-(4-{4-[2-(6-{1-[6-(2-hydroxyphenyl)pyridazin-4-yl]-4-phenylpiperidine-4-carbonyl}-2,6-diazaspiro[3.3]heptan-2-yl)-2-oxoethyl]piperazin-1-yl}-3-methyl-2-oxo-2,3-dihydro-1H-1,3-benzodiazol-1-yl)piperidine-2,6-dione

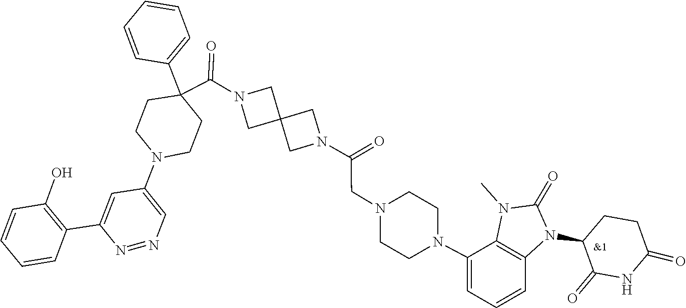 [SMILES: Cn1c(=O)n([C@H]2CCC(=O)NC2=O)c2cccc(N3CCN(CC(=O)N4CC5(C4)CN(C(=O)C4(c6ccccc6)CCN(c6cnnc(-c7ccccc7O)c6)CC4)C5)CC3)c21]

Combined a solution of 2-[5-(4-{2,6-diazaspiro[3.3]heptane-2-carbonyl}-4-phenylpiperidin-1-yl)pyridazin-3-yl]phenol; trifluoroacetic acid (19.00 mg, 0.0334 mmol) in 100 μL DMA, with N,N-diisopropylethylamine (34.96 μL, 0.03 g, 0.2001 mmol) and a solution of rac-(4-{1-[(3R)-2,6-dioxopiperidin-3-yl]-3-methyl-2-oxo-1,3-benzodiazol-4-yl}piperazin-1-yl)acetic acid (14.73 mg, 0.0367 mmol) (synthesis as described in WO2024039901 A2 2024-02-22) in 100 μL DMA and then added a solution of [(dimethylamino)({[1,2,3]triazolo[4,5-b]pyridin-3-yloxy})methylidene]dimethylazanium; hexafluoro-lambda5-phosphanuide (13.95 mg, 0.0367 mmol) in 100 μL DMA and stirred at rt for 2 h. Purified the solution directly by RP-HPLC to yield the title compound (0.0021 g, 7.04%). LCMS: C₄₆H₅₀N₁₀O₆ requires: 838.4. found: m/z=839.3 [M+H]⁺.

### Example 127

### rac-(3R)-3-(4-{3-[(7-{1-[6-(2-hydroxyphenyl)pyridazin-4-yl]-4-phenylpiperidine-4-carbonyl}-2,7-diazaspiro[3.5]nonan-2-yl)methyl]-2-oxa-8-azaspiro[4.5]decan-8-yl}phenyl)piperidine-2,6-dione

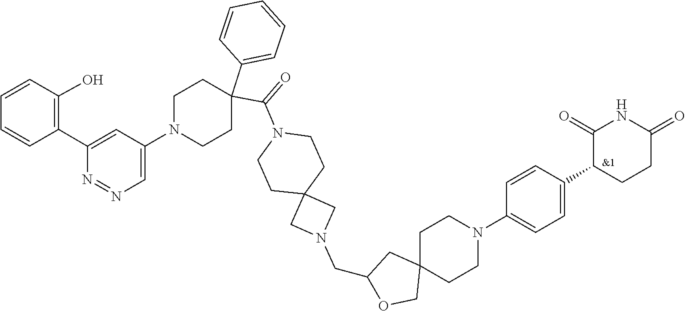 [SMILES: O=C1CC[C@H](c2ccc(N3CCC4(CC3)COC(CN3CC5(CCN(C(=O)C6(c7ccccc7)CCN(c7cnnc(-c8ccccc8O)c7)CC6)CC5)C3)C4)cc2)C(=O)N1]

Combined 2-[5-(4-{2,7-diazaspiro[3.5]nonane-7-carbonyl}-4-phenylpiperidin-1-yl)pyridazin-3-yl]phenol; trifluoroacetic acid (20.5 mg, 343 mmol) and rac-8-{4-[(3R)-2,6-dioxopiperidin-3-yl]phenyl}-2-oxa-8-azaspiro[4.5]decane-3-carbaldehyde (13.5 mg, 377 mmol) in DMA (0.20 mL) with N,N-diisopropylethylamine (29.9 μL, 0.02 g, 172 mmol) and then added sodium triacetoxyborohydride (21.81 mg, 0.1029 mmol) and stirred for 2 h. Purified the solution directly by RP-HPLC to yield the title compound (12.3 mg, 43.3%). LCMS: C₄₉H₅₇N₇O₅ requires: 823.4. found: m/z=824.4 [M+H]^{+ 1}H NMR (500 MHz, DMSO) δ 10.77 (s, 1H), 8.93 (d, J=2.9 Hz, 1H), 8.14 (s, 1H), 8.13-8.08 (m, 1H), 7.52 (d, J=3.0 Hz, 1H), 7.43-7.25 (m, 6H), 7.03 (d, J=8.5 Hz, 2H), 6.95-6.86 (m, 4H), 4.12 (d, J=13.1 Hz, 2H), 3.88 (s, 1H), 3.72 (dd, J=10.9, 5.0 Hz, 1H), 3.52 (d, J=8.5 Hz, 1H), 3.46 (d, J=8.5 Hz, 1H), 3.19-3.08 (m, 6H), 3.07 (s, 1H), 2.61 (dd, J=11.4, 5.4 Hz, 1H), 2.49-2.42 (m, 3H), 2.34 (s, 1H), 2.15-2.06 (m, 1H), 2.03 (s, 1H), 1.86 (dd, J=12.0, 6.6 Hz, 1H), 1.59 (s, 3H), 1.32 (dd, J=12.5, 8.5 Hz, 1H).

### Example 128

### rac-3-(4-(4-(2-(7-(1-(6-(2-hydroxyphenyl)pyridazin-4-yl)-4-((1-methyl-1h-pyrazol-3-yl)oxy)piperidine-4-carbonyl)-2,7-diazaspiro[3.5]nonan-2-yl)ethyl)piperidin-1-yl)phenyl)piperidine-2,6-dione

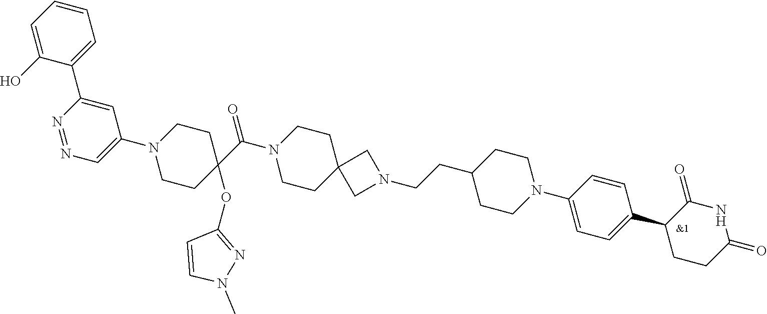 [SMILES: Cn1ccc(OC2(C(=O)N3CCC4(CC3)CN(CCC3CCN(c5ccc([C@@H]6CCC(=O)NC6=O)cc5)CC3)C4)CCN(c3cnnc(-c4ccccc4O)c3)CC2)n1]

### Step 1: Synthesis of (1-(6-(2-hydroxyphenyl)pyridazin-4-yl)-4-((1-methyl-1H-pyrazol-3-yl)oxy)piperidin-4-yl)(2,7-diazaspiro[3.5]nonan-7-yl)methanone

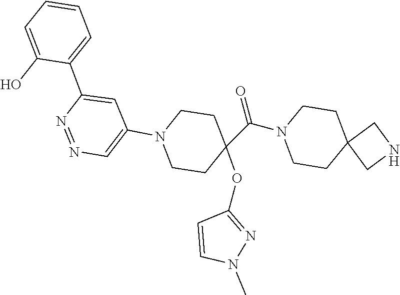 [SMILES: Cn1ccc(OC2(C(=O)N3CCC4(CC3)CNC4)CCN(c3cnnc(-c4ccccc4O)c3)CC2)n1]

1-(6-(2-hydroxyphenyl)pyridazin-4-yl)-4-((1-methyl-1H-pyrazol-3-yl)oxy)piperidine-4-carboxylic acid (55.63 mg, 0.1407 mmol) and tert-butyl 2,7-diazaspiro[3.5]nonane-2-carboxylate (31.84 mg, 0.1407 mmol) were dissolved in 1.0 mL DMA. N,N-diisopropylethylamine (0.07 mL, 54.55 mg, 0.4221 mmol), followed by addition of [(dimethylamino)({[1,2,3]triazolo[4,5-b]pyridin-3-yloxy})methylidene]dimethylazanium; hexafluoro-lambda5-phosphanuide (80.24 mg, 0.2110 mmol in one portion and the resulting solution was stirred at room temperature until complete by LCMS. Once complete, the reaction was quenched with 2 mL H₂O and extracted three times with EtOAc. The organic extracts were combined, dried over Na₂S₂O₄ and concentrated in vacuo. The crude material was dissolved in 1:1 TFA:DCM and stirred for 1 h, then concentrated and lyophilized to use in the next step. LCMS: C₂₇H₃₃N₇O₃ requires 503.2. found: m/z=504.3 [M+H]⁺.

### Step 2: Synthesis of the Title Compound

(1-(6-(2-hydroxyphenyl)pyridazin-4-yl)-4-((1-methyl-1H-pyrazol-3-yl)oxy)piperidin-4-yl)(2,7-diazaspiro[3.5]nonan-7-yl)methanone (11.1 mg, 0.022 mmol) and rac-1-(4-(2,6-dioxopiperidin-3-yl)phenyl)piperidine-4-carbaldehyde (6.91 mg, 0.022 mmol) were dissolved in 0.5 mL DMA. N,N-diisopropylethylamine (14.3 mg, 0.110 mmol), followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H2O to yield the title compound (9.0 mg, 51.0%). LCMS: C₄₅H₅₅N₉O₅ requires 801.4. found: m/z=802.3 [M+H]⁺.

### Example 129

### (3RS&)-3-{4-[(2rs,4sr)-2-(7-{1-[6-(2-hydroxyphenyl)pyridazin-4-yl]-4-phenylpiperidine-4-carbonyl}-2,7-diazaspiro[3.5]nonan-2-yl)-6-azaspiro[3.4]octan-6-yl]phenyl}piperidine-2,6-dione

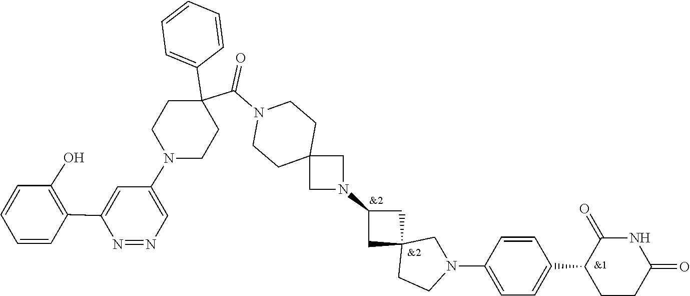 [SMILES: O=C1CC[C@H](c2ccc(N3CC[C@]4(C3)C[C@@H](N3CC5(CCN(C(=O)C6(c7ccccc7)CCN(c7cnnc(-c8ccccc8O)c7)CC6)CC5)C3)C4)cc2)C(=O)N1]

Combined 2-[5-(4-{2,7-diazaspiro[3.5]nonane-7-carbonyl}-4-phenylpiperidin-1-yl)pyridazin-3-yl]phenol; trifluoroacetic acid (18.00 mg, 0.0301 mmol) and rac-(3R)-3-(4-{2-oxo-6-azaspiro[3.4]octan-6-yl}phenyl)piperidine-2,6-dione (10.35 mg, 0.0331 mmol) in DMA (0.40 mL) with N,N-diisopropylethylamine (26.23 μL, 0.02 g, 0.1506 mmol) and then added sodium triacetoxyborohydride (19.15 mg, 0.0904 mmol) and stirred for 1 h. Purified the solution directly by RP-HPLC to yield the title compound (0.0095 g, 39.83%). LCMS: C₄₇H₅₃N₇O₄ requires: 779.4, found: m/z=780.3 [M+H]⁺.

### Example 130

### rac-N-{1-[2-(1-{1-[(3r)-2,6-dioxopiperidin-3-yl]-3-methyl-2-oxo-2,3-dihydro-1H-1,3-benzodiazol-4-yl}piperidin-4-yl)ethyl]piperidin-4-yl}-1-[6-(2-hydroxyphenyl)pyridazin-4-yl]-N-methyl-4-phenylpiperidine-4-carboxamide

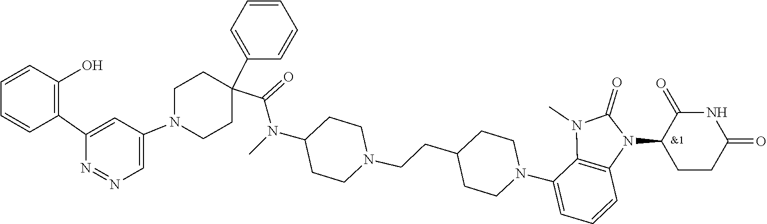 [SMILES: CN(C(=O)C1(c2ccccc2)CCN(c2cnnc(-c3ccccc3O)c2)CC1)C1CCN(CCC2CCN(c3cccc4c3n(C)c(=O)n4[C@@H]3CCC(=O)NC3=O)CC2)CC1]

Combined 1-[6-(2-hydroxyphenyl)pyridazin-4-yl]-N-methyl-4-phenyl-N-(piperidin-4-yl)piperidine-4-carboxamide; bis(trifluoroacetic acid) (16.50 mg, 0.0236 mmol) and rac-2-(1-{1-[(3R)-2,6-dioxopiperidin-3-yl]-3-methyl-2-oxo-1,3-benzodiazol-4-yl}piperidin-4-yl)acetaldehyde (9.97 mg, 0.0259 mmol) in DMA (0.40 mL) with N,N-diisopropylethylamine (20.54 μL, 0.02 g, 0.1179 mmol) and then added sodium triacetoxyborohydride (14.99 mg, 0.0707 mmol) and stirred for 1 h. Purified the solution directly by RP-HPLC to yield the title compound (0.012 g, 58.45%). LCMS: C₄₈H₅₇N₉O₅ requires: 839.4. found: m/z=840.4 [M+H]⁺.

### Example 131

### rac-(3R)-3-(4-{4-[2-(6-{1-[6-(2-hydroxyphenyl)pyridazin-4-yl]-4-phenylpiperidine-4-carbonyl}-2,6-diazaspiro[3.3]heptan-2-yl)ethyl]-4-methylpiperidin-1-yl}phenyl)piperidine-2,6-dione

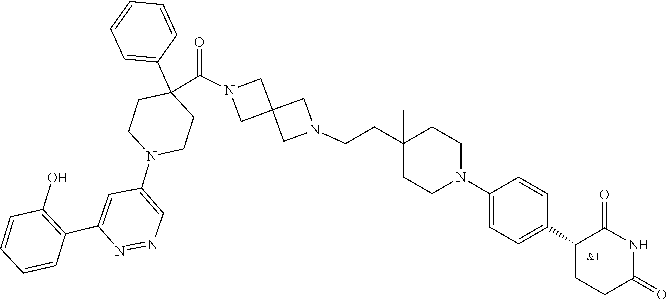 [SMILES: CC1(CCN2CC3(C2)CN(C(=O)C2(c4ccccc4)CCN(c4cnnc(-c5ccccc5O)c4)CC2)C3)CCN(c2ccc([C@H]3CCC(=O)NC3=O)cc2)CC1]

Combined 2-[5-(4-{2,6-diazaspiro[3.3]heptane-2-carbonyl}-4-phenylpiperidin-1-yl)pyridazin-3-yl]phenol; trifluoroacetic acid (14.50 mg, 0.0255 mmol) and rac-2-(1-{4-[(3R)-2,6-dioxopiperidin-3-yl]phenyl}-4-methylpiperidin-4-yl)acetaldehyde (9.20 mg, 0.0280 mmol) in DMA (0.40 mL) with N,N-diisopropylethylamine (22.17 μL, 0.02 g, 0.1273 mmol) and then added sodium triacetoxyborohydride (16.19 mg, 0.0764 mmol) and stirred for 1 h. Purified the solution directly by RP-HPLC to yield the title compound (0.0079 g, 40.00%). LCMS: C₄₆H₅₃N₇O₄ requires: 767.4, found: m/z=768.3 [M+H]⁺.

### Example 132

### rac-(3R)-3-{4-[4-(2-{6-[4-(2-fluorophenyl)-1-[6-(2-hydroxyphenyl)pyridazin-4-yl]piperidine-4-carbonyl]-2,6-diazaspiro[3.3]heptan-2-yl}ethyl)piperidin-1-yl]phenyl}piperidine-2,6-dione

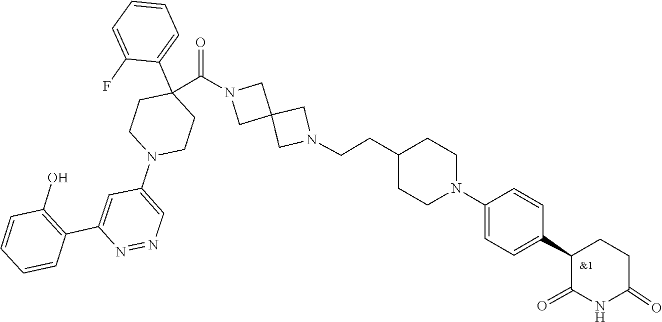 [SMILES: O=C1CC[C@H](c2ccc(N3CCC(CCN4CC5(C4)CN(C(=O)C4(c6ccccc6F)CCN(c6cnnc(-c7ccccc7O)c6)CC4)C5)CC3)cc2)C(=O)N1]

### Step 1: Synthesis of 2-[5-(4-{2,6-diazaspiro[3.3]heptane-2-carbonyl}-4-(2-fluorophenyl)piperidin-1-yl)pyridazin-3-yl]phenol

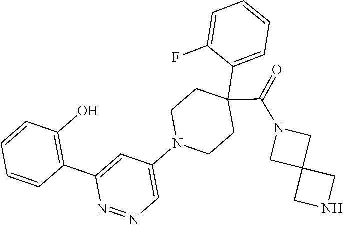 [SMILES: O=C(N1CC2(CNC2)C1)C1(c2ccccc2F)CCN(c2cnnc(-c3ccccc3O)c2)CC1]

This intermediate was synthesized according to Step 1 of Example 85 using 4-(2-fluorophenyl)-1-[6-(2-hydroxyphenyl)pyridazin-4-yl]piperidine-4-carboxylic acid (Intermediate 9) and tert-butyl 2,6-diazaspiro[3.3]heptane-2-carboxylate. LCMS: C₂₇H₂₈N₅O₂ requires: 473.2, found: m/z=474.2 [M+H]⁺.

### Step 2: Synthesis of the Title Compound

Combined 2-[5-(4-{2,6-diazaspiro[3.3]heptane-2-carbonyl}-4-(2-fluorophenyl)piperidin-1-yl)pyridazin-3-yl]phenol; trifluoroacetic acid (20.00 mg, 0.0331 mmol) and rac-2-(1-{4-[(3R)-2,6-dioxopiperidin-3-yl]phenyl}piperidin-4-yl)acetaldehyde (11.46 mg, 0.0364 mmol) in DMA (0.20 mL) with N,N-diisopropylethylamine (28.86 μL, 0.02 g, 0.1657 mmol) and then added sodium triacetoxyborohydride (21.07 mg, 0.0994 mmol) and stirred for 2 h. Purified the solution directly by RP-HPLC to yield the title compound (0.007 g, 26.71%). LCMS: C₄₅H₅₀FN₇O₄ requires: 771.4, found: m/z=772.3 [M+H]⁺.

### Example 133

### N-{1-[(1-{4-[(3R)-2,6-dioxopiperidin-3-yl]phenyl}piperidin-4-yl)methyl]piperidin-4-yl}-1-[6-(2-hydroxyphenyl)pyridazin-4-yl]-4-(2-methoxyphenyl)-n-methylpiperidine-4-carboxamide

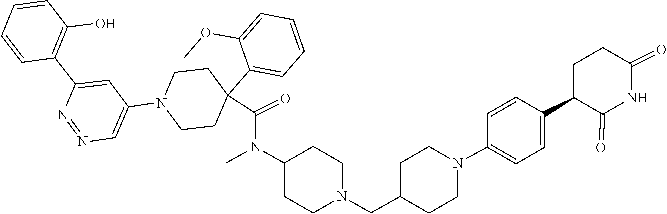 [SMILES: COc1ccccc1C1(C(=O)N(C)C2CCN(CC3CCN(c4ccc([C@H]5CCC(=O)NC5=O)cc4)CC3)CC2)CCN(c2cnnc(-c3ccccc3O)c2)CC1]

Combined 1-[6-(2-hydroxyphenyl)pyridazin-4-yl]-4-(2-methoxyphenyl)-N-methyl-N-(piperidin-4-yl)piperidine-4-carboxamide; trifluoroacetic acid (20.01 mg, 0.0332 mmol) and 1-{4-[(3R)-2,6-dioxopiperidin-3-yl]phenyl}piperidine-4-carbaldehyde (10.95 mg, 0.0365 mmol) in DMA (0.20 mL) with N,N-diisopropylethylamine (28.87 μL, 0.02 g, 0.1658 mmol) and then added sodium triacetoxyborohydride (21.08 mg, 0.0995 mmol) and stirred for 2 h. Purified the solution directly by RP-HPLC to yield the title compound (0.0076 g, 29.17%). LCMS: C₄₆H₅₅N₇O₅ requires: 785.4, found: m/z=786.4 [M+H]⁺.

### Example 134

### rac-N-{1-[2-(1-{4-[(3R)-2,6-dioxopiperidin-3-yl]phenyl}piperidin-4-yl)ethyl]piperidin-4-yl}-4-(4-fluorophenyl)-1-[6-(2-hydroxyphenyl)pyridazin-4-yl]-n-methylpiperidine-4-carboxamide

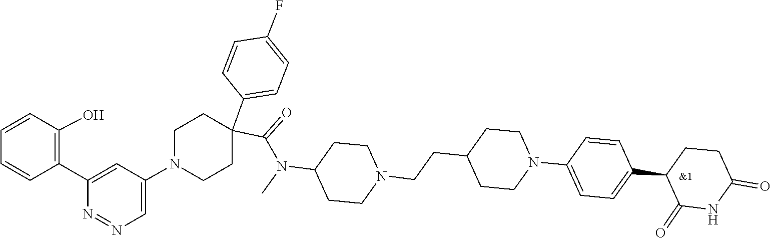 [SMILES: CN(C(=O)C1(c2ccc(F)cc2)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of 4-(4-fluorophenyl)-1-[6-(2-hydroxyphenyl)pyridazin-4-yl]-N-methyl-N-(piperidin-4-yl)piperidine-4-carboxamide

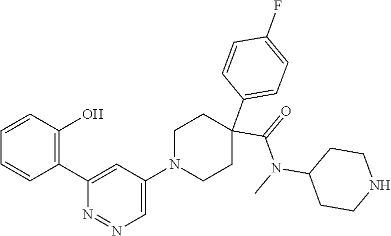 [SMILES: CN(C(=O)C1(c2ccc(F)cc2)CCN(c2cnnc(-c3ccccc3O)c2)CC1)C1CCNCC1]

This intermediate was synthesized according to Step 1 of Example 85 using 4-(4-fluorophenyl)-1-[6-(2-hydroxyphenyl)pyridazin-4-yl]piperidine-4-carboxylic acid (Intermediate 8).

LCMS: C₂₈H₃₂N₅O₂ requires: 489.3. found: m/z=490.4 [M+H]⁺.

### Example 135

### (s)-3-(4-(4-((4-(((1-(6-(2-hydroxyphenyl)pyridazin-4-yl)-4-phenylpiperidin-4-yl)methyl)amino)piperidin-1-yl)methyl)piperidin-1-yl)phenyl)piperidine-2,6-dione

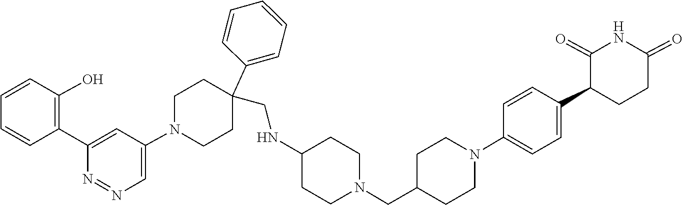 [SMILES: O=C1CC[C@@H](c2ccc(N3CCC(CN4CCC(NCC5(c6ccccc6)CCN(c6cnnc(-c7ccccc7O)c6)CC5)CC4)CC3)cc2)C(=O)N1]

### Step 1: Synthesis of 2-(5-(4-phenyl-4-((piperidin-4-ylamino)methyl)piperidin-1-yl)pyridazin-3-yl)phenol

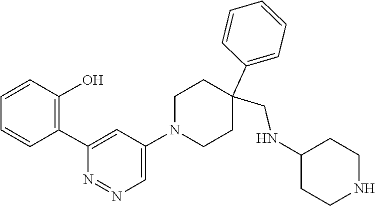 [SMILES: Oc1ccccc1-c1cc(N2CCC(CNC3CCNCC3)(c3ccccc3)CC2)cnn1]

tert-Butyl 4-[({1-[6-(2-hydroxyphenyl)pyridazin-4-yl]-4-phenylpiperidin-4-yl}methyl)amino]piperidine-1-carboxylate (110.0 mg, mmol) was dissolved in DCM (1.00 mL) followed by the addition of TFA (0.30 mL). The mixture was stirred until complete by LCMS. Then, it was concentrated to dryness and used in the next step without further purification. LCMS: C₂₇H₃₃N₅O requires 443.3. found: m/z=444.3 [M+H]⁺.

### Step 2: Synthesis of the Title Compound

To a solution of 2-(5-{4-phenyl-4-[(piperidin-4-ylamino)methyl]piperidin-1-yl}pyridazin-3-yl)phenol; trifluoroacetic acid (30.00 mg, 0.0538 mmol) in THF (0.90 mL) and 1-{4-[(3R)-2,6-dioxopiperidin-3-yl]phenyl}piperidine-4-carbaldehyde (19.39 mg, 0.0646 mmol) was added acetic acid (3.08 μL, 0.00 g, 0.0538 mmol) and stirred for 30 min. Then sodium cyanoborohydride (10.14 mg, 0.1614 mmol) was added and stirred until complete by LCMS. The solution was injected onto RP-FC and purified with a gradient of 5-95% MeCN in H₂O to furnish the title compound (1.98 mg, 5.06% Yield). LCMS: C₄₄H₅₃N₇O₃ requires 727.4. found: m/z=728.5 [M+H]⁺.

### Step 2: Synthesis of the Title Compound

Combined 4-(4-fluorophenyl)-1-[6-(2-hydroxyphenyl)pyridazin-4-yl]-N-methyl-N-(piperidin-4-yl)piperidine-4-carboxamide; trifluoroacetic acid (20.00 mg, 0.0331 mmol) and rac-2-(1-{4-[(3R)-2,6-dioxopiperidin-3-yl]phenyl}piperidin-4-yl)acetaldehyde (11.46 mg, 0.0364 mmol) in DMA (0.20 mL) with N,N-diisopropylethylamine (28.86 μL, 0.02 g, 0.1657 mmol) and then added sodium triacetoxyborohydride (21.07 mg, 0.0994 mmol) and stirred for 2 h. Purified the solution directly by RP-HPLC to yield the title compound (0.0064 g, 24.15%). LCMS: C₄₆H₅₄FN₇04 requires: 787.4. found: m/z=788.3 [M+H]⁺.

### Example 136

### rac-(3R)-3-(2,6-difluoro-4-{4-[(2-{1-[6-(2-hydroxyphenyl)pyridazin-4-yl]-4-phenoxypiperidine-4-carbonyl}-2,7-diazaspiro[3.5]nonan-7-yl)methyl]piperidin-1-yl}phenyl)piperidine-2,6-dione

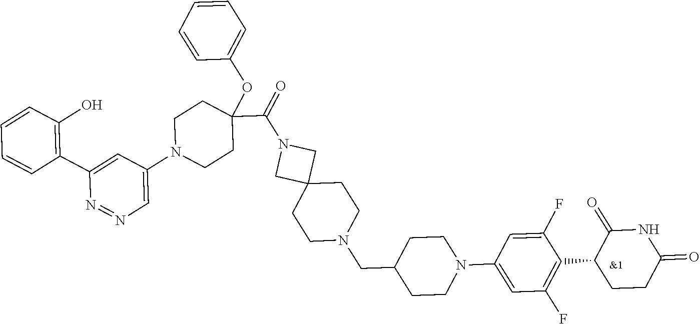 [SMILES: O=C1CC[C@H](c2c(F)cc(N3CCC(CN4CCC5(CC4)CN(C(=O)C4(Oc6ccccc6)CCN(c6cnnc(-c7ccccc7O)c6)CC4)C5)CC3)cc2F)C(=O)N1]

### Step 1: Synthesis of 2-[5-(4-{2,7-diazaspiro[3.5]nonane-2-carbonyl}-4-phenoxypiperidin-1-yl)pyridazin-3-yl]phenol

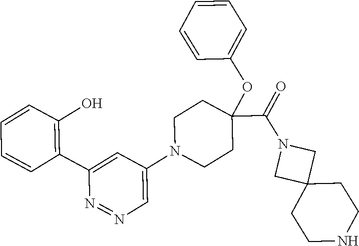 [SMILES: O=C(N1CC2(CCNCC2)C1)C1(Oc2ccccc2)CCN(c2cnnc(-c3ccccc3O)c2)CC1]

A solution of 1-[6-(2-hydroxyphenyl)pyridazin-4-yl]-4-phenoxypiperidine-4-carboxylic acid (Intermediate 20) (15.00 mg, 0.0383 mmol) in 0.2 mL DMA was added to tert-butyl 2,7-diazaspiro[3.5]nonane-7-carboxylate (10.41 mg, 0.0460 mmol) with N,N-diisopropylethylamine (20.03 μL, 0.01 g, 0.1150 mmol) and then [(dimethylamino)({[1,2,3]triazolo[4,5-b]pyridin-3-yloxy})methylidene]dimethylazanium; hexafluoro-lambda5-phosphanuide (17.49 mg, 0.0460 mmol) was added as a solution in 0.2 mL DMA. Stirred at rt. Diluted with EtOAc (3 mL) and washed with 1M citric acid (2×4 mL) and bicarb (1×4 ml), then passed through a phase separator. Concentrated to dryness under vacuum. This material was dissolved in DCM (1 mL) and TFA (1 mL) and after 1 hour, it was concentrated by rotary evaporation, and then lyophilized twice from 1:1 ACN/water. Used in the next step as is without further purification. LCMS: C₂₉H₃₃N₅O₃ requires: 499.3, found: m/z=500.6 [M+H]⁺.

### Step 2: Synthesis of the Title Compound

Combined a solution of 2-[5-(4-{2,7-diazaspiro[3.5]nonane-2-carbonyl}-4-phenoxypiperidin-1-yl)pyridazin-3-yl]phenol; trifluoroacetic acid (20.01 mg, 0.0326 mmol) in 0.1 mL DMA to a solution of rac-1-{4-[(3R)-2,6-dioxopiperidin-3-yl]-3,5-difluorophenyl}piperidine-4-carbaldehyde (12.06 mg, 0.0359 mmol) (synthesis as described in WO2024039901 A2 2024-02-22) in 0.1 mL DMA with N,N-diisopropylethylamine (34.08 μL, 0.03 g, 0.1957 mmol) and then added a solution of sodium triacetoxyborohydride (20.73 mg, 0.0978 mmol) in 0.1 mL DMA and stirred overnight. Purified the solution directly by RP-HPLC to yield the title compound (0.0104 g, 38.27%). LCMS: C₄₆H₅₁F₂N₇O₅ requires: 819.4. found: m/z=820.3 [M+H]⁺.

### Example 137

### rac-(3R)-3-(4-{2-[(7-{1-[6-(2-hydroxyphenyl)pyridazin-4-yl]-4-phenylpiperidine-4-carbonyl}-2,7-diazaspiro[3.5]nonan-2-yl)methyl]-7-azaspiro[3.5]nonan-7-yl}phenyl)piperidine-2,6-dione

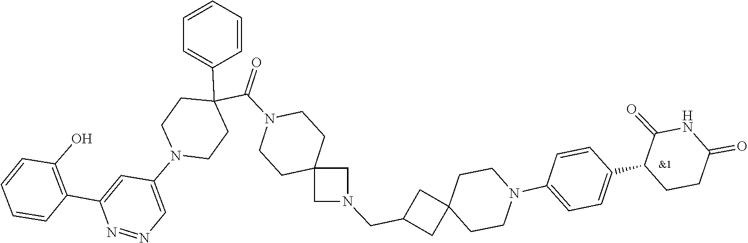 [SMILES: O=C1CC[C@H](c2ccc(N3CCC4(CC3)CC(CN3CC5(CCN(C(=O)C6(c7ccccc7)CCN(c7cnnc(-c8ccccc8O)c7)CC6)CC5)C3)C4)cc2)C(=O)N1]

### Step 1: Synthesis of 2-[5-(4-{2,7-diazaspiro[3.5]nonane-2-carbonyl}-4-phenylpiperidin-1-yl)pyridazin-3-yl]phenol

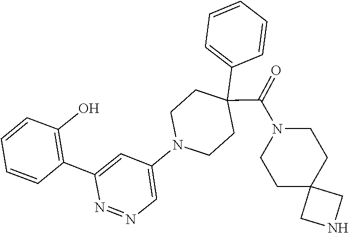 [SMILES: O=C(N1CCC2(CC1)CNC2)C1(c2ccccc2)CCN(c2cnnc(-c3ccccc3O)c2)CC1]

This intermediate was synthesized according to step 1 of Example 98 using 1-[6-(2-hydroxyphenyl)pyridazin-4-yl]-4-phenylpiperidine-4-carboxylic acid and tert-butyl 2,7-diazaspiro[3.5]nonane-2-carboxylate. LCMS: C₂₉H₃₃N₅O₂ requires: 483.3. found: m/z=484.4 [M+H]⁺.

### Step2: Synthesis of the Title Compound

Combined 2-[5-(4-{2,7-diazaspiro[3.5]nonane-7-carbonyl}-4-phenylpiperidin-1-yl)pyridazin-3-yl]phenol; trifluoroacetic acid (18.00 mg, 0.0301 mmol) and rac-7-{4-[(3R)-2,6-dioxopiperidin-3-yl]phenyl}-7-azaspiro[3.5]nonane-2-carbaldehyde (11.28 mg, 0.0331 mmol) in DMA (0.40 mL) with N,N-diisopropylethylamine (26.23 μL, 0.02 g, 0.1506 mmol) and then added sodium triacetoxyborohydride (19.15 mg, 0.0904 mmol) and stirred for 1 h. Purified the solution directly by RP-HPLC to yield the title compound (0.0078 g, 30.77%). LCMS: C₄₉H₅₇N₇O₄ requires: 807.4. found: m/z=808.3 [M+H]⁺.

### Example 138

### rac-(3R)-3-(2,3-difluoro-4-{4-[(6-{1-[6-(2-hydroxyphenyl)pyridazin-4-yl]-4-phenylpiperidine-4-carbonyl}-2,6-diazaspiro[3.3]heptan-2-yl)methyl]piperidin-1-yl}phenyl)piperidine-2,6-dione

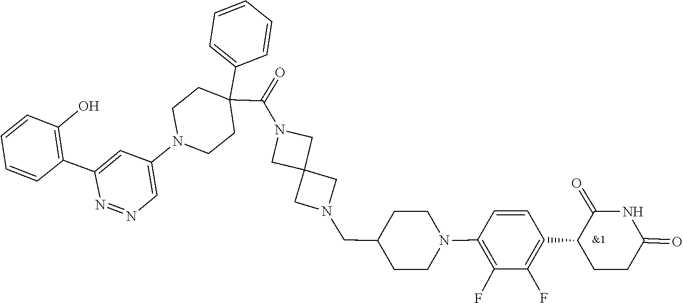 [SMILES: O=C1CC[C@H](c2ccc(N3CCC(CN4CC5(C4)CN(C(=O)C4(c6ccccc6)CCN(c6cnnc(-c7ccccc7O)c6)CC4)C5)CC3)c(F)c2F)C(=O)N1]

This compound was synthesis according to Example 131 using rac-(R)-1-(4-(2,6-dioxopiperidin-3-yl)-2,3-difluorophenyl)piperidine-4-carbaldehyde. LCMS: C₄₄H₄₇F₂N₇O₄ requires: 775.4. found: m/z=776.3 [M+H]⁺.

### Example 139

### N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-4-(3-methoxyphenyl)-N-methylpiperidine-4-carboxamide

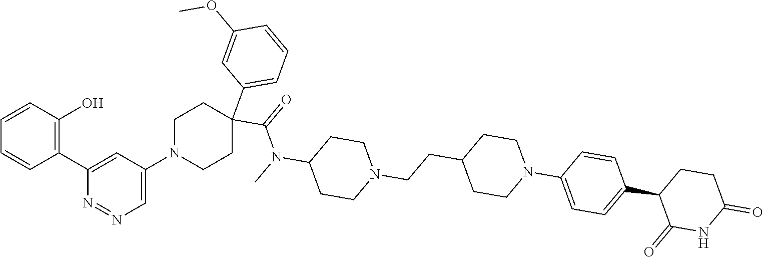 [SMILES: COc1cccc(C2(C(=O)N(C)C3CCN(CCC4CCN(c5ccc([C@H]6CCC(=O)NC6=O)cc5)CC4)CC3)CCN(c3cnnc(-c4ccccc4O)c3)CC2)c1]

### Step 1: Synthesis of 1-[6-(2-hydroxyphenyl)pyridazin-4-yl]-4-(3-methoxyphenyl)-N-methyl-N-(piperidin-4-yl)piperidine-4-carboxamide

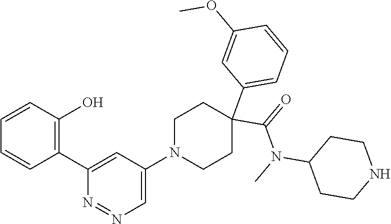 [SMILES: COc1cccc(C2(C(=O)N(C)C3CCNCC3)CCN(c3cnnc(-c4ccccc4O)c3)CC2)c1]

To a solution of 1-[6-(2-hydroxyphenyl)pyridazin-4-yl]-4-(3-methoxyphenyl)piperidine-4-carboxylic acid (Intermediate 7) (103.00 mg, 0.2540 mmol) in 1.6 mL DMA was added tert-butyl 4-(methylamino)piperidine-1-carboxylate (163.33 mg, 0.7621 mmol) and N,N-diisopropylethylamine (132.75 μL, 0.10 g, 0.7621 mmol). Cooled in an ice bath then [(dimethylamino)({[1,2,3]triazolo[4,5-b]pyridin-3-yloxy})methylidene]dimethylazanium; hexafluoro-lambda5-phosphanuide (144.89 mg, 0.3811 mmol) was added. Stirred at rt for 4 days. Precipitated out the product by adding the reaction mixture into 20 mL water. Filtered to collect the solid, rinsing with water (2×5 mL). Dried the solid under vacuum to yield 140 mg of the crude Boc protected material. This was dissolved in DCM (1 mL) and TFA (1 mL) and after 1 hour, it was concentrated by rotary evaporation, and then lyophilized twice from 1:1 ACN/water. Used in the next step as is without further purification. LCMS: C₂₉H₃₅N₅O₂ requires: 501.3. found: m/z=502.4 [M+H]⁺.

### Step 2: Synthesis of the Title Compound

Combined 1-[6-(2-hydroxyphenyl)pyridazin-4-yl]-4-(3-methoxyphenyl)-N-methyl-N-(piperidin-4-yl)piperidine-4-carboxamide; trifluoroacetic acid (10.00 mg, 0.0162 mmol) and 2-(1-{4-[(3R)-2,6-dioxopiperidin-3-yl]phenyl}piperidin-4-yl)acetaldehyde (5.62 mg, 0.0179 mmol) in DMA (0.20 mL) with N,N-diisopropylethylamine (14.15 μL, 0.01 g, 0.0812 mmol) and then added sodium triacetoxyborohydride (10.33 mg, 0.0487 mmol) and stirred for 2 h. Purified the solution directly by RP-HPLC to yield the title compound (0.0037 g, 28.02%). LCMS: C₄₇H₅₇N₇O₅ requires: 799.4, found: m/z=800.4 [M+H]^{+ 1}H NMR (500 MHz, DMSO) Î′ 10.77 (s, 1H), 8.92 (s, 1H), 8.16-8.08 (m, 1H), 7.51 (d, J=2.9 Hz, 1H), 7.37-7.29 (m, 2H), 7.03 (d, J=8.5 Hz, 2H), 6.96-6.85 (m, 6H), 6.78 (s, 1H), 4.10 (s, 1H), 3.75 (s, 3H), 3.72 (dd, J=10.9, 4.9 Hz, 2H), 3.64 (s, 1H), 2.68 (s, 1H), 2.61 (dd, J=11.3, 5.4 Hz, 2H), 2.49-2.43 (m, 5H), 2.42 (s, 2H), 2.15-2.06 (m, 2H), 2.04-1.97 (m, 2H), 1.72 (s, 4H), 1.36 (s, 2H), 1.24 (s, 2H), 0.90 (s, 1H).

### Example 140

### N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N-methyl-4-(pyridin-4-yl)piperidine-4-carboxamide

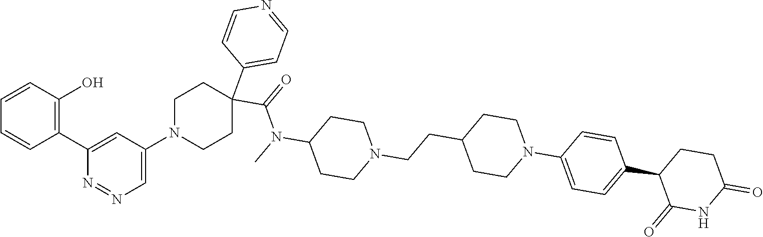 [SMILES: CN(C(=O)C1(c2ccncc2)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of 1-[6-(2-hydroxyphenyl)pyridazin-4-yl]-N-methyl-N-(piperidin-4-yl)-4-(pyridin-4-yl)piperidine-4-carboxamide

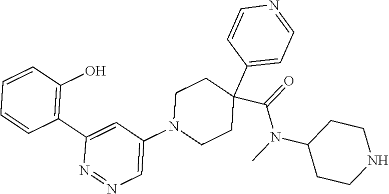 [SMILES: CN(C(=O)C1(c2ccncc2)CCN(c2cnnc(-c3ccccc3O)c2)CC1)C1CCNCC1]

This intermediate was synthesis according to step 1 of example 139 using 1-[6-(2-hydroxyphenyl)pyridazin-4-yl]-4-(pyridin-4-yl)piperidine-4-carboxylic acid (Intermediate 6). LCMS: C₂₇H₃₂N₆O₂ requires: 472.3. found: m/z=473.4

### Step 2: Synthesis of the Title Compound

Combined 1-[6-(2-hydroxyphenyl)pyridazin-4-yl]-N-methyl-N-(piperidin-4-yl)-4-(pyridin-4-yl)piperidine-4-carboxamide; trifluoroacetic acid (9.70 mg, 0.0165 mmol) and 2-(1-{4-[(3R)-2,6-dioxopiperidin-3-yl]phenyl}piperidin-4-yl)acetaldehyde (5.72 mg, 0.0182 mmol) in DMA (0.20 mL) with N,N-diisopropylethylamine (14.40 μL, 0.01 g, 0.0827 mmol) and then added sodium triacetoxyborohydride (10.51 mg, 0.0496 mmol) and stirred for 2 h. Purified the solution directly by RP-HPLC to yield the title compound (0.006 g, 39.63%) LCMS: C₄₅H₅₄N₈O₄ requires: 770.4, found: m/z=771.4 [M+H]^{+ 1}H NMR (500 MHz, DMSO) δ 10.78 (s, 1H), 8.96 (s, 1H), 8.60 (d, J=5.5 Hz, 2H), 7.50 (s, 1H), 7.39 (s, 1H), 7.30 (s, 1H), 7.04 (d, J=8.4 Hz, 2H), 6.99 (s, 1H), 6.90 (d, J=8.3 Hz, 2H), 4.54 (s, 1H), 4.16 (s, 2H), 3.72 (dd, J=11.0, 5.0 Hz, 1H), 3.67 (d, J=12.1 Hz, 2H), 3.56 (s, 1H), 3.10 (s, 1H), 2.73-2.58 (m, 5H), 2.50-2.43 (m, 8H), 2.42 (s, 1H), 2.38 (s, 1H), 2.20-2.08 (m, 3H), 2.04-1.95 (m, 2H), 1.82 (s, 2H), 1.75 (s, 1H), 1.61 (s, 1H), 1.44 (s, 1H), 1.28 (d, J=12.8 Hz, 2H).

### Example 141

### (3RS&)-3-(6-{4-[(1RS)-1-(6-{1-[6-(2-hydroxyphenyl)pyridazin-4-yl]-4-phenylpiperidine-4-carbonyl}-2,6-diazaspiro[3.3]heptan-2-yl)ethyl]piperidin-1-yl}pyridin-3-yl)piperidine-2,6-dione

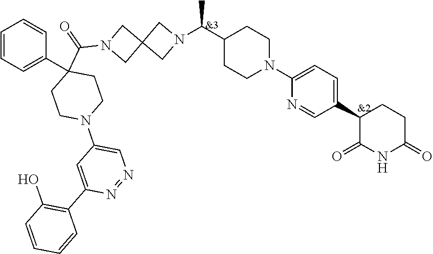 [SMILES: C[C@@H](C1CCN(c2ccc([C@H]3CCC(=O)NC3=O)cn2)CC1)N1CC2(CN(C(=O)C3(c4ccccc4)CCN(c4cnnc(-c5ccccc5O)c4)CC3)C2)C1]

LCMS: C₄₄H₅₀N₈O₄ requires: 754.4. found: m/z=755.3 [M+H]⁺

### Example 142

### rac-(r)-3-(4-(4-(4-(((1-(6-(2-hydroxyphenyl)pyridazin-4-yl)-4-phenylpiperidin-4-yl)methyl)(methyl)amino)piperidine-1-carbonyl)piperidin-1-yl)phenyl)piperidine-2,6-dione

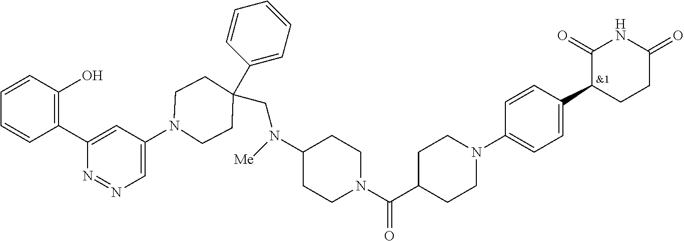 [SMILES: CN(CC1(c2ccccc2)CCN(c2cnnc(-c3ccccc3O)c2)CC1)C1CCN(C(=O)C2CCN(c3ccc([C@@H]4CCC(=O)NC4=O)cc3)CC2)CC1]

The title compound was synthesized according to Step 143 of Example 2 using rac-(R)-1-(4-(2,6-dioxopiperidin-3-yl)phenyl)piperidine-4-carboxylic acid hydrochloride and 2-(5-(4-((methyl(piperidin-4-yl)amino)methyl)-4-phenylpiperidin-1-yl)pyridazin-3-yl)phenol. The solution was injected onto RP-FC and purified with a gradient of 5-95% MeCN in H₂O to furnish the title compound (0.6 mg, 1% yield). LCMS: C₄₅H₅₃N₇O₄ requires 755.4. found: m/z=756.4 [M+H]⁺.

### Example 143

### rac-(r)-3-(4-(1-(2-(4-(((1-(6-(2-hydroxyphenyl)pyridazin-4-yl)-4-phenylpiperidin-4-yl)methyl)(methyl)amino)piperidin-1-yl)-2-oxoethyl)piperidin-4-yl)phenyl)piperidine-2,6-dione

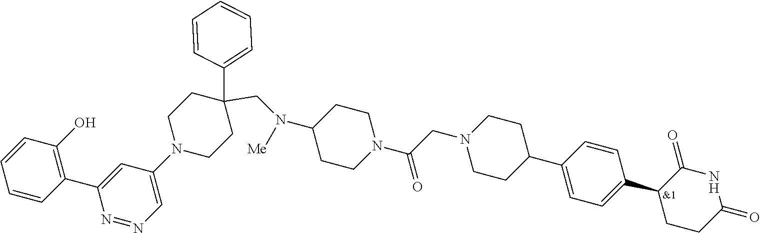 [SMILES: CN(CC1(c2ccccc2)CCN(c2cnnc(-c3ccccc3O)c2)CC1)C1CCN(C(=O)CN2CCC(c3ccc([C@@H]4CCC(=O)NC4=O)cc3)CC2)CC1]

### Step 1: Synthesis of tert-butyl 4-(((1-(6-(2-hydroxyphenyl)pyridazin-4-yl)-4-phenylpiperidin-4-yl)methyl)amino)piperidine-1-carboxylate

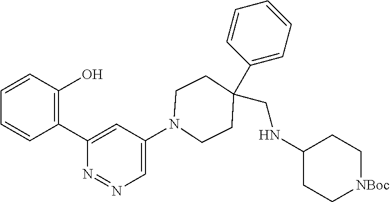 [SMILES: CC(C)(C)OC(=O)N1CCC(NCC2(c3ccccc3)CCN(c3cnnc(-c4ccccc4O)c3)CC2)CC1]

tert-Butyl 4-oxopiperidine-1-carboxylate (82.91 mg, 0.4161 mmol) was added to a solution of 2-{5-[4-(aminomethyl)-4-phenylpiperidin-1-yl]pyridazin-3-yl}phenol (50.00 mg, 0.1387 mmol) in DCE (1.50 mL). It was subsequently treated with acetic acid (23.80 μL, 0.02 g, 0.4161 mmol) and stirred for 2 h. Then, sodium triacetoxyborohydride (88.20 mg, 0.4161 mmol) was added and the reaction mixture was stirred until complete by LCMS. The reaction mixture was dissolved with DCM and quenched with a saturated solution of NaHCO₃, extracted with DCM, dried over Na₂SO₄, filtered and the solvent was removed under reduced pressure. The crude material was used in the next step without further purification. LCMS: C₃₂H₄₁N₅O₃ requires 543.3. found: m/z=544.3 [M+H]⁺.

### Step 2: Synthesis of 2-(5-(4-((methyl(piperidin-4-yl)amino)methyl)-4-phenylpiperidin-1-yl)pyridazin-3-yl)phenol

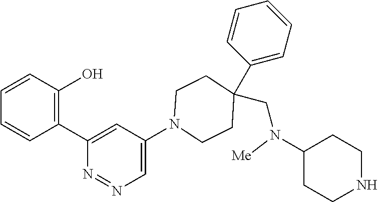 [SMILES: CN(CC1(c2ccccc2)CCN(c2cnnc(-c3ccccc3O)c2)CC1)C1CCNCC1]

tert-Butyl 4-[({1-[6-(2-hydroxyphenyl)pyridazin-4-yl]-4-phenylpiperidin-4-yl}methyl)amino]piperidine-1-carboxylate (115.00 mg, 0.2115 mmol) was dissolved in DCE (3.00 mL) and a 37% aqueous solution of formaldehyde (18.90 μL, 0.02 g, 0.2538 mmol) and triethylamine (58.64 μL, 0.04 g, 0.4230 mmol) were added. After stirring at room temperature for 30 minutes, sodium triacetoxyborohydride (134.48 mg, 0.6345 mmol) was added and the reaction stirred at room temperature until complete by LCMS. The reaction mixture was dissolved with MeOH filtered and the solvent was removed under reduced pressure. Then, the crude material was dissolved in a mixture of DCM (4.00 mL) and TFA (1.20 mL), and the mixture was stirred until complete by LCMS. Then, it was concentrated to dryness and used in the next step without further purification. LCMS: C₂₈H₃₅N₅₀ requires 457.3. found: m/z=458.2 [M+H]⁺.

### Step 3: Synthesis of the Title Compound

To a solution of 2-[5-(4-{[methyl(piperidin-4-yl)amino]methyl}-4-phenylpiperidin-1-yl)pyridazin-3-yl]phenol; trifluoroacetic acid (50.00 mg, 0.0875 mmol) and rac-(R)-2-(4-(4-(2,6-dioxopiperidin-3-yl)phenyl)piperidin-1-yl)acetic acid hydrochloride (48.13 mg, 0.1312 mmol) in DMA (0.80 mL) was added N,N-diisopropylethylamine (152.77 μL, 0.11 g, 0.8747 mmol). Then, [(dimethylamino)({[1,2,3]triazolo[4,5-b]pyridin-3-yloxy})methylidene]dimethylazanium; hexafluoro-lambda5-phosphanuide (66.52 mg, 0.1749 mmol) was added and the reaction mixture was stirred at room temperature until complete by LCMS. The solution was injected onto RP-FC and purified with a gradient of 5-95% MeCN in H₂O to furnish the title compound (10.6 mg, 13.6%). LCMS: C₄₆H₅₅N₇O₄ requires 769.4. found: m/z=770.4 [M+H]⁺.

### Example 144

### rac-3-(2-(1-(4-((r)-2,6-dioxopiperidin-3-yl)phenyl)piperidin-4-yl)ethyl)-n-((1-(6-(2-hydroxyphenyl)pyridazin-4-yl)-4-phenylpiperidin-4-yl)methyl)-3-azabicyclo[3.2.1]octane-8-carboxamide

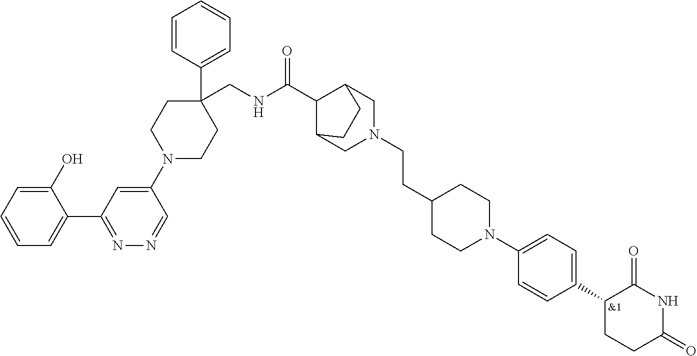 [SMILES: O=C1CC[C@H](c2ccc(N3CCC(CCN4CC5CCC(C4)C5C(=O)NCC4(c5ccccc5)CCN(c5cnnc(-c6ccccc6O)c5)CC4)CC3)cc2)C(=O)N1]

### Step 1: Synthesis of tert-butyl 8-(((1-(6-(2-hydroxyphenyl)pyridazin-4-yl)-4-phenylpiperidin-4-yl)methyl)carbamoyl)-3-azabicyclo[3.2.1]octane-3-carboxylate

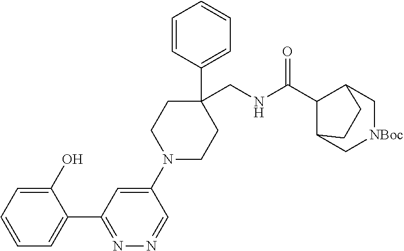 [SMILES: CC(C)(C)OC(=O)N1CC2CCC(C1)C2C(=O)NCC1(c2ccccc2)CCN(c2cnnc(-c3ccccc3O)c2)CC1]

This intermediate was synthesized as described for the Example 104 Step 1, using 3-(tert-butoxycarbonyl)-3-azabicyclo[3.2.1]octane-8-carboxylic acid in place of 1-(tert-butoxycarbonyl)-9-oxa-1-azaspiro[5.5]undecane-4-carboxylic acid. LCMS C₃₅H₄₃N₅O₄ requires 597.3, found m/z=598.3 [M+H]⁺.

### Step 2: Synthesis of N-((1-(6-(2-hydroxyphenyl)pyridazin-4-yl)-4-phenylpiperidin-4-yl)methyl)-3-azabicyclo[3.2.1]octane-8-carboxamide

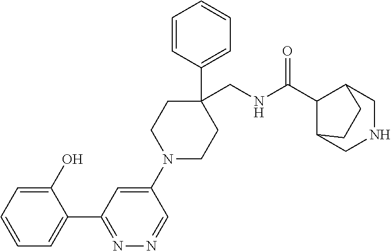 [SMILES: O=C(NCC1(c2ccccc2)CCN(c2cnnc(-c3ccccc3O)c2)CC1)C1C2CCC1CNC2]

This intermediate was synthesized as described for the Example 104 Step 2, using tert-butyl 8-(((1-(6-(2-hydroxyphenyl)pyridazin-4-yl)-4-phenylpiperidin-4-yl)methyl)carbamoyl)-3-azabicyclo[3.2.1]octane-3-carboxylate in place of tert-butyl 4-(((1-(6-(2-hydroxyphenyl)pyridazin-4-yl)-4-phenylpiperidin-4-yl)methyl)carbamoyl)-9-oxa-1-azaspiro[5.5]undecane-1-carboxylate. LCMS C₃₇H₄₇N₅O₅ requires 597.2, found m/z=598.2 [M+H]⁺.

### Step 3: Synthesis of the Title Compound

The title compound was synthesized as described for the Example 104 Step 3, using N-((1-(6-(2-hydroxyphenyl)pyridazin-4-yl)-4-phenylpiperidin-4-yl)methyl)-3-azabicyclo[3.2.1]octane-8-carboxamide in place of N-((1-(6-(2-hydroxyphenyl)pyridazin-4-yl)-4-phenylpiperidin-4-yl)methyl)-9-oxa-1-azaspiro[5.5]undecane-4-carboxamide (8.6 mgs, 58% over three steps). LCMS C₄₈H₅₇N₇O₄ requires 795.4, found m/z=796.4 [M+H]⁺.

### Example 145

### rac-(r)-1-(2-(1-(4-(2,6-dioxopiperidin-3-yl)phenyl)piperidin-4-yl)ethyl)-n-((1-(6-(2-hydroxyphenyl)pyridazin-4-yl)-4-phenylpiperidin-4-yl)methyl)-n-(2-methoxyethyl)piperidine-4-carboxamide

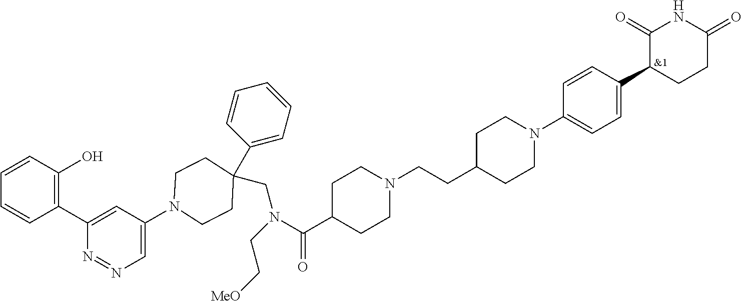 [SMILES: COCCN(CC1(c2ccccc2)CCN(c2cnnc(-c3ccccc3O)c2)CC1)C(=O)C1CCN(CCC2CCN(c3ccc([C@@H]4CCC(=O)NC4=O)cc3)CC2)CC1]

### Step 1: Synthesis of 2-(5-(4-(((2-methoxyethyl)amino)methyl)-4-phenylpiperidin-1-yl)pyridazin-3-yl)phenol

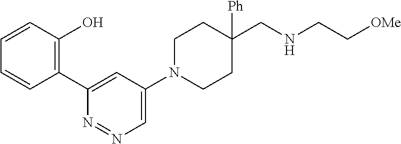 [SMILES: COCCNCC1(c2ccccc2)CCN(c2cnnc(-c3ccccc3O)c2)CC1]

To a vial containing 1-[6-(2-hydroxyphenyl)pyridazin-4-yl]-4-phenylpiperidine-4-carbaldehyde (50.00 mg, 0.1391 mmol) in 1,2-DCE (1.50 mL) was added 2-methoxyethanamine (19.61 μL, 22.55 mg, 0.2782 mmol) and acetic acid (11.93 μL, 0.01 g, 0.209 mmol) and the reaction was allowed to stir at room temperature for 30 minutes. After 30 minutes, sodium triacetoxyborohydride (58.97 mg, 0.2782 mmol) was added in one portion and the reaction was allowed to stir at room temperature until complete by LCMS. The reaction mixture was dissolved with DCM and quenched with a saturated solution of NaHCO₃, extracted with DCM, dried over Na₂SO₄, filtered and the solvent was removed under reduced pressure. The material was used in the next step without further purification. LCMS: C₂₅H₃₀N₄O₂ requires 418.2. found: m/z=419.2 [M+H]⁺.

### Step 2: Synthesis of N-((1-(6-(2-hydroxyphenyl)pyridazin-4-yl)-4-phenylpiperidin-4-yl)methyl)-N-(2-methoxyethyl)piperidine-4-carboxamide

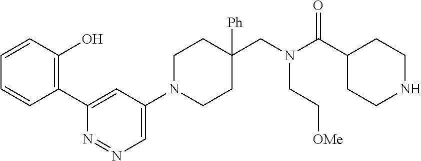 [SMILES: COCCN(CC1(c2ccccc2)CCN(c2cnnc(-c3ccccc3O)c2)CC1)C(=O)C1CCNCC1]

Combined 2-[5-(4-{[(2-methoxyethyl)amino]methyl}-4-phenylpiperidin-1-yl)pyridazin-3-yl]phenol (10.00 mg, 0.0250 mmol) and 1-(tert-butoxycarbonyl)piperidine-4-carboxylic acid (9.16 mg, 0.0399 mmol) in DMA (0.40 mL) and added N,N-diisopropylethylamine (13.08 μL, 0.01 g, 0.0749 mmol). Added [(dimethylamino)({[1,2,3]triazolo[4,5-b]pyridin-3-yloxy})methylidene]dimethylazanium; hexafluoro-lambda5-phosphanuide (18.99 mg, 0.0499 mmol) and stirred at 60° C. until complete by LCMS. Diluted with DCM (1 mL) and washed with water (2 mL), then with a saturated solution NaHCO₃ (2 mL), extracted with DCM, dried over Na₂SO₄, filtered and the solvent removed under reduced pressure. The crude material was purified by flash chromatography on silica with a gradient of 0-100% EtOAc in Hexanes to afford the Boc protected intermediate (6.0 mg, 38% yield). LCMS: C₃₆H₄₇N₅O₅ requires 629.4. found: m/z=630.4 [M+H]⁺.

Then, the material was dissolved in DCM (1.00 mL) and TFA (0.3 mL) was added and stirred until complete by LCMS. Then, it was concentrated to dryness and used in the next step without further purification. LCMS: C₃₁H₃₉N₅O₃ requires 529.3. found: m/z=530.2 [M+H]⁺.

### Step 3: Synthesis of the Title Compound

Combined a solution of N-({1-[6-(2-hydroxyphenyl)pyridazin-4-yl]-4-phenylpiperidin-4-yl}methyl)-N-(2-methoxyethyl)piperidine-4-carboxamide; trifluoroacetic acid (7.00 mg, 0.0109 mmol) in DMA (0.30 mL) with 2-{1-[4-(2,6-dioxopiperidin-3-yl)phenyl]piperidin-4-yl}acetaldehyde (3.77 mg, 0.0120 mmol) and N,N-diisopropylethylamine (9.49 μL, 0.01 g, 0.0545 mmol) and stirred for 30 min. Then, added sodium triacetoxyborohydride (6.93 mg, 0.0327 mmol) and stirred until complete by LCMS. The solution was injected onto RP-FC and purified with a gradient of 5-95% MeCN in H₂O to furnish the title compound (2.64 mg, 26%). LCMS: C₄₉H₆₁N₇O₅ requires 827.4. found: m/z=828.5 [M+H]⁺.

### Example 146

### rac-5-(2-(1-(4-((r)-2,6-dioxopiperidin-3-yl)phenyl)piperidin-4-yl)ethyl)-n-((1-(6-(2-hydroxyphenyl)pyridazin-4-yl)-4-phenylpiperidin-4-yl)methyl)-n-methyl-5-azaspiro[3.5]nonane-8-carboxamide

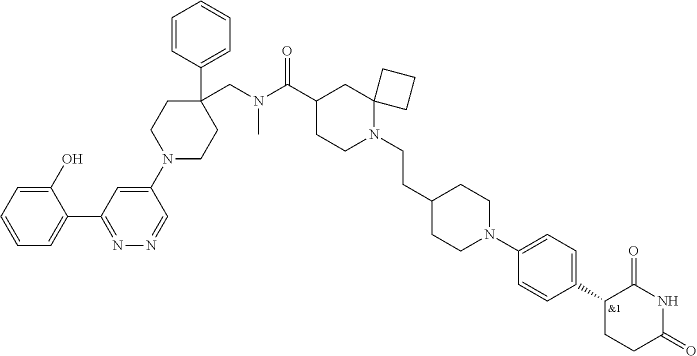 [SMILES: CN(CC1(c2ccccc2)CCN(c2cnnc(-c3ccccc3O)c2)CC1)C(=O)C1CCN(CCC2CCN(c3ccc([C@H]4CCC(=O)NC4=O)cc3)CC2)C2(CCC2)C1]

### Step 1: Synthesis of 2-(5-(4-((methylamino)methyl)-4-phenylpiperidin-1-yl)pyridazin-3-yl)phenol

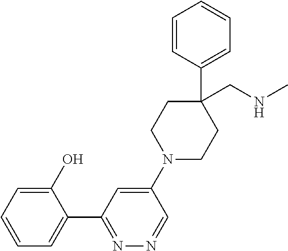 [SMILES: CNCC1(c2ccccc2)CCN(c2cnnc(-c3ccccc3O)c2)CC1]

A vial was charged with 1-(6-(2-hydroxyphenyl)pyridazin-4-yl)-4-phenylpiperidine-4-carbaldehyde (101 mg, 0.281 mmol), methylamine (281 μL, 0.562 mmol, 2M in THF), acetic acid (32.1 μL, 0.562 mmol), and 1,2-DCE (1.4 mL) and the reaction mixture was allowed to stir at RT for 30 minutes. After 30 minutes, sodium triacetoxyborohydride (89.3 mg, 0.562 mmol) was added in one portion and the mixture was allowed to stir until completion as judged by LCMS. Once complete, the reaction was diluted by addition of DCM and neutralized via addition of NaHCO₃ (sat aq.). The mixture was extracted with DCM (3×10 mL) and the combined organic layers were washed with brine (30 mL), dried with anhydrous MgSO₄, filtered, and concentrated to afford the compound as crude, light-yellow solid which was taken forward without any further purification. LCMS C₂₃H₂₆N₄₀ requires: 374.5. found: m/z=375.2 [M+H]⁺.

### Step 2: Synthesis of tert-butyl 8-(((1-(6-(2-hydroxyphenyl)pyridazin-4-yl)-4-phenylpiperidin-4-yl)methyl)(methyl)carbamoyl)-5-azaspiro[3.5]nonane-5-carboxylate

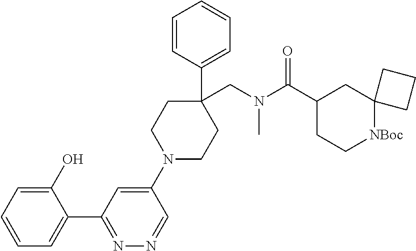 [SMILES: CN(CC1(c2ccccc2)CCN(c2cnnc(-c3ccccc3O)c2)CC1)C(=O)C1CCN(C(=O)OC(C)(C)C)C2(CCC2)C1]

To a vial containing 5-(tert-butoxycarbonyl)-5-azaspiro[3.5]nonane-8-carboxylic acid (15.1 mgs, 0.056 mmol) was added DMF (0.25 mL) followed by addition of DIPEA (13.4 μL, 0.077 mmol) and HATU (21.3 mgs, 0.056 mmol). In a separate vial, 2-(5-(4-((methylamino)methyl)-4-phenylpiperidin-1-yl)pyridazin-3-yl)phenol (19.1 mgs, 0.051 mmol) was dissolved in DMF (0.25 mL) and was added to the acid-containing vial after 10 minutes of stirring. Once the amine was added, the reaction mixture was allowed to stir until completion as judged by LCMS. Once complete, the reaction mixture was diluted with DCM and water, followed by quenching via addition of NaHCO₃ (sat aq.). The reaction mixture was allowed to stir for 5 minutes, and the mixture was then transferred to a phase separator cartridge, with elution of the organic layer. The collected organic layer was dried with anhydrous MgSO₄, filtered and concentrated to afford crude material as a clear-colorless oil, which was taken forward without purification. LCMS C₃₇H₄₇N₅O₄ requires: 625.3. found: m/z=626.3 [M+H]⁺.

### Step 3: Synthesis of N-((1-(6-(2-hydroxyphenyl)pyridazin-4-yl)-4-phenylpiperidin-4-yl)methyl)-N-methyl-5-azaspiro[3.5]nonane-8-carboxamide

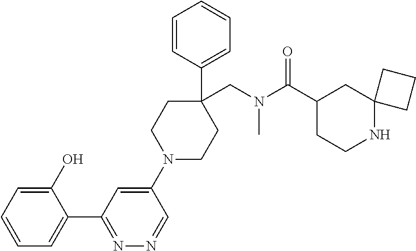 [SMILES: CN(CC1(c2ccccc2)CCN(c2cnnc(-c3ccccc3O)c2)CC1)C(=O)C1CCNC2(CCC2)C1]

To a vial containing tert-butyl 8-(((1-(6-(2-hydroxyphenyl)pyridazin-4-yl)-4-phenylpiperidin-4-yl)methyl)(methyl)carbamoyl)-5-azaspiro[3.5]nonane-5-carboxylate (17.8 mgs, 0.028 mmol) was added THF (0.5 mL) followed by addition of HCl (113.8 μL, 0.284 mmol, 4M in dioxane) in one portion and the reaction was allowed to stir until completion as judged by LCMS. Once complete the reaction was concentrated to remove solvent and volatiles, afford crude material as a light-yellow oil (HCl salt), which was taken forward without any further purification. LCMS C₃₂H₃₉N₅O₂ requires: 525.3, found m/z=526.3 [M+H]⁺.

### Step 4: Synthesis of the Title Compound

To a vial containing N-((1-(6-(2-hydroxyphenyl)pyridazin-4-yl)-4-phenylpiperidin-4-yl)methyl)-N-methyl-5-azaspiro[3.5]nonane-8-carboxamide hydrochloride (15 mgs, 0.028 mmol) was added DIPEA (24.9 μL, 0.143 mmol), followed by addition of rac-(R)-2-(1-(4-(2,6-dioxopiperidin-3-yl)phenyl)piperidin-4-yl)acetaldehyde (9.96 mgs, 0.032 mmol) and the mixture was allowed to stir 30 minutes. After 30 minutes, sodium triacetoxyborohydride (18.1 mgs, 0.086 mmol) was added to the vial in one portion and the reaction mixture was allowed to stir until completion as judged by LCMS. Once complete, the reaction mixture was directly injected onto RP-FC and purified with a gradient of 5-95% MeCN in H₂O to furnish the title compound (11.9 mgs, 50% over four steps). LCMS C₅₀H₆₁N₇O₄ requires 823.4, found m/z=824.4 [M+H]⁺.

### Example 147

### 1-[6-(2-hydroxyphenyl)pyridazin-4-yl]-4-phenyl-N-[(1,4s)-4-{[1-(1-{5-[(3rs)-2,6-dioxopiperidin-3-yl]pyridin-2-yl}piperidin-4-yl)ethyl]amino}cyclohexyl]piperidine-4-carboxamide

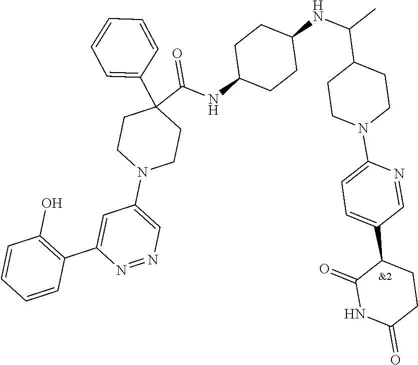 [SMILES: CC(N[C@H]1CC[C@@H](NC(=O)C2(c3ccccc3)CCN(c3cnnc(-c4ccccc4O)c3)CC2)CC1)C1CCN(c2ccc([C@H]3CCC(=O)NC3=O)cn2)CC1]

### Example 148

### N-{1-[(3S)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methyl-4-phenylpiperidine-4-carboxamide

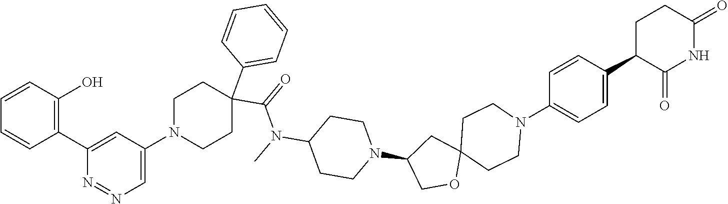 [SMILES: CN(C(=O)C1(c2ccccc2)CCN(c2cnnc(-c3ccccc3O)c2)CC1)C1CCN([C@@H]2COC3(CCN(c4ccc([C@H]5CCC(=O)NC5=O)cc4)CC3)C2)CC1]

Title compound was P1 from stage two (65.9 mg, 12.40%) LCMS: C₄₇H₅₅N₇O₅ requires: 797.4. found: m/z=798.4 [M+H]⁺.

¹H NMR (500 MHz, DMSO) δ 14.64 (s, 1H), 10.76 (s, 1H), 8.91 (d, J=2.7 Hz, 1H), 8.10 (dd, J=8.5, 1.7 Hz, 1H), 7.51 (d, J=2.8 Hz, 1H), 7.39 (d, J=7.5 Hz, 2H), 7.36-7.24 (m, 4H), 7.05-6.99 (m, 2H), 6.95-6.89 (m, 2H), 6.87 (d, J=8.7 Hz, 2H), 4.10 (d, J=13.1 Hz, 2H), 3.86 (d, J=38.7 Hz, 1H), 3.71 (dd, J=11.0, 4.9 Hz, 1H), 3.39 (t, J=13.2 Hz, 3H), 3.15 (s, 4H), 2.91 (s, 1H), 2.75 (s, 1H), 2.67 (s, 2H), 2.60 (dd, J=11.4, 5.4 Hz, 1H), 2.45 (dt, J=17.2, 4.4 Hz, 2H), 2.17-2.07 (m, 1H), 2.04-1.96 (m, 3H), 1.90 (s, 1H), 1.59 (s, 6H), 1.45 (s, 1H), 1.41 (s, 1H), 0.84 (s, 1H).

### Example 149

### N-{1-[(3R)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methyl-4-phenylpiperidine-4-carboxamide

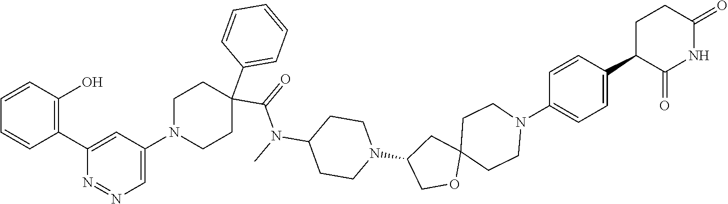 [SMILES: CN(C(=O)C1(c2ccccc2)CCN(c2cnnc(-c3ccccc3O)c2)CC1)C1CCN([C@H]2COC3(CCN(c4ccc([C@H]5CCC(=O)NC5=O)cc4)CC3)C2)CC1]

The Title compound was P2 from stage two (0.0758 g, 14.02%) LCMS: C₄₇H₅₅N₇O₅ requires: 797.4. found: m/z=798.4 [M+H]⁺.

¹H NMR (500 MHz, DMSO) δ 14.64 (s, 1H), 10.76 (s, 1H), 8.91 (d, J=2.8 Hz, 1H), 8.10 (dd, J=8.5, 1.7 Hz, 1H), 7.51 (d, J=2.8 Hz, 1H), 7.39 (d, J=7.7 Hz, 2H), 7.36-7.24 (m, 4H), 7.05-6.99 (m, 2H), 6.95-6.84 (m, 4H), 4.13-4.07 (m, 2H), 3.82 (s, 1H), 3.71 (dd, J=11.0, 4.9 Hz, 1H), 3.38 (t, J=12.7 Hz, 3H), 3.15 (s, 2H), 2.91 (s, 1H), 2.75 (s, 1H), 2.67 (s, 2H), 2.60 (dd, J=11.4, 5.4 Hz, 1H), 2.45 (dt, J=17.2, 4.5 Hz, 2H), 2.17-2.06 (m, 2H), 2.00 (dq, J=13.2, 5.0 Hz, 3H), 1.90 (s, OH), 1.59 (s, 6H), 1.46 (d, J=11.6 Hz, 1H), 1.41 (d, J=11.5 Hz, 1H), 0.84 (s, 1H).

### Example 150

### (3RS)-3-{4-[(3aR,6aR)-5-[2-(2-{1-[6-(2-hydroxyphenyl)pyridazin-4-yl]-4-phenylpiperidine-4-carbonyl}-2,7-diazaspiro[3.5]nonan-7-yl)-2-oxoethyl]-octahydropyrrolo[3,4-c]pyrrol-2-yl]phenyl}piperidine-2,6-dione

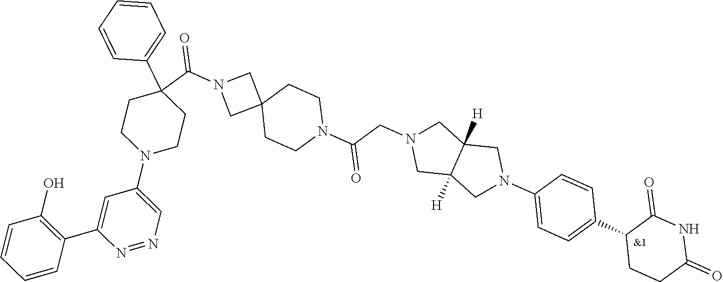 [SMILES: O=C1CC[C@H](c2ccc(N3C[C@H]4CN(CC(=O)N5CCC6(CC5)CN(C(=O)C5(c7ccccc7)CCN(c7cnnc(-c8ccccc8O)c7)CC5)C6)C[C@@H]4C3)cc2)C(=O)N1]

Combined a solution of 2-[5-(4-{2,7-diazaspiro[3.5]nonane-2-carbonyl}-4-phenylpiperidin-1-yl)pyridazin-3-yl]phenol (8.01 mg, 0.0166 mmol) in 300 μL DMA, with N,N-diisopropylethylamine (23.14 μL, 0.02 g, 0.1325 mmol) and [(3aR,6aR)-5-{4-[(3RS)-2,6-dioxopiperidin-3-yl]phenyl}-hexahydropyrrolo[3,4-c]pyrrol-2-yl]acetic acid (7.70 mg, 0.0215 mmol) and then added a solution of [(dimethylamino)({[1,2,3]triazolo[4,5-b]pyridin-3-yloxy})methylidene]dimethylazanium; hexafluoro-lambda5-phosphanuide (8.19 mg, 0.0215 mmol) in 0.1 mL DMA and stirred at rt overnight. Purified the solution directly by RP-HPLC to yield the title compound (0.0024 g, 17.10%) LCMS: C₄₈H₅₄N₈O₅ requires: 822.4. found: m/z=823.4 [M+H]⁺.

### Example 151

### rac-N-{1-[2-(1-{4-[(3R)-2,6-dioxopiperidin-3-yl]-3-fluorophenyl}piperidin-4-yl)ethyl]piperidin-4-yl}-1-[6-(2-hydroxyphenyl)pyridazin-4-yl]-N-methyl-4-phenylpiperidine-4-carboxamide

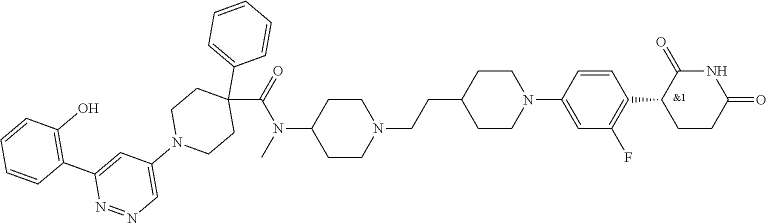 [SMILES: CN(C(=O)C1(c2ccccc2)CCN(c2cnnc(-c3ccccc3O)c2)CC1)C1CCN(CCC2CCN(c3ccc([C@H]4CCC(=O)NC4=O)c(F)c3)CC2)CC1]

Combined 1-[6-(2-hydroxyphenyl)pyridazin-4-yl]-N-methyl-4-phenyl-N-(piperidin-4-yl)piperidine-4-carboxamide; trifluoroacetic acid (24.00 mg, 0.0410 mmol) and rac-2-(1-{4-[(3R)-2,6-dioxopiperidin-3-yl]-3-fluorophenyl}piperidin-4-yl)acetaldehyde (14.5 mg, 045 mmol) in DMA (0.40 mL) with N,N-diisopropylethylamine (35.69 μL, 0.03 g, 205 mmol) and then added sodium triacetoxyborohydride (26.1 mg, 123 mmol) and stirred for 1 h. Purified the solution directly by RP-HPLC to yield the title compound (0.0158 g, 46.87%). LCMS: C₄₆H₅₄FN₇O₄ requires: 787.4, found: m/z=788.3 [M+H]⁺.

### Example 152

### rac-(3R)-3-(4-{9-[2-(2-{1-[6-(2-hydroxyphenyl)pyridazin-4-yl]-4-phenylpiperidine-4-carbonyl}-2,7-diazaspiro[3.5]nonan-7-yl)-2-oxoethyl]-1-oxa-4,9-diazaspiro[5.5]undecan-4-yl}phenyl)piperidine-2,6-dione

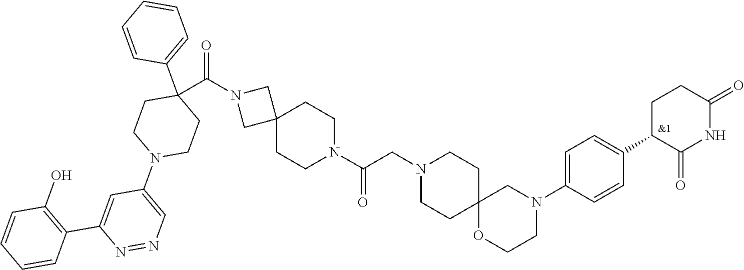 [SMILES: O=C1CC[C@@H](c2ccc(N3CCOC4(CCN(CC(=O)N5CCC6(CC5)CN(C(=O)C5(c7ccccc7)CCN(c7cnnc(-c8ccccc8O)c7)CC5)C6)CC4)C3)cc2)C(=O)N1]

Combined a solution of 2-[5-(4-{2,7-diazaspiro[3.5]nonane-2-carbonyl}-4-phenylpiperidin-1-yl)pyridazin-3-yl]phenol (8.01 mg, 0.0166 mmol) in 300 μL DMA, with N,N-diisopropylethylamine (23.14 μL, 0.02 g, 0.1325 mmol) and rac-(4-{4-[(3R)-2,6-dioxopiperidin-3-yl]phenyl}-1-oxa-4,9-diazaspiro[5.5]undecan-9-yl)acetic acid (8.64 mg, 0.0215 mmol) and then added a solution of [(dimethylamino)({[1,2,3]triazolo[4,5-b]pyridin-3-yloxy})methylidene]dimethylazanium; hexafluoro-lambda5-phosphanuide (8.19 mg, 0.0215 mmol) in 0.1 mL DMA and stirred at rt overnight. Purified the solution directly by RP-HPLC to yield (0.0052 g, 35.77%). LCMS: C₅₀H₅₈N₈O₆ requires: 866.4. found: m/z=867.4 [M+H]⁺.

### Example 153

### N-[(4RS)-1-[(1-{4-[(3R)-2,6-dioxopiperidin-3-yl]phenyl}piperidin-4-yl)methyl]-1-azaspiro[5.5]undecan-4-yl]-1-[6-(2-hydroxyphenyl)pyridazin-4-yl]-N-methyl-4-phenylpiperidine-4-carboxamide

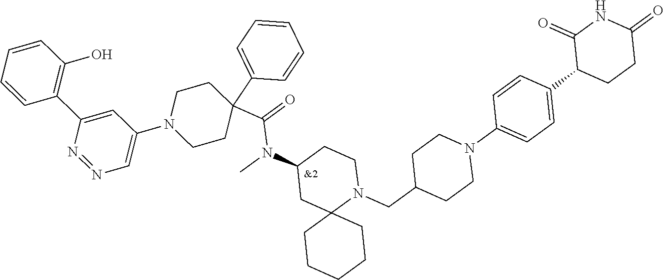 [SMILES: CN(C(=O)C1(c2ccccc2)CCN(c2cnnc(-c3ccccc3O)c2)CC1)[C@H]1CCN(CC2CCN(c3ccc([C@H]4CCC(=O)NC4=O)cc3)CC2)C2(CCCCC2)C1]

### Step 1: Synthesis of tert-butyl 4-(N-methyl-1-{6-[2-(methoxymethoxy)phenyl]pyridazin-4-yl}-4-phenylpiperidine-4-amido)-1-azaspiro[5.5]undecane-1-carboxylate

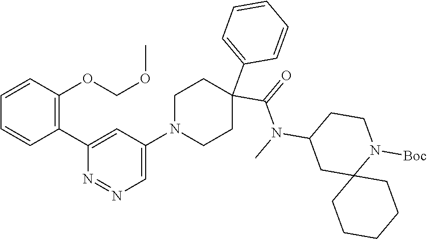 [SMILES: COCOc1ccccc1-c1cc(N2CCC(C(=O)N(C)C3CCN(C(=O)OC(C)(C)C)C4(CCCCC4)C3)(c3ccccc3)CC2)cnn1]

1-{6-[2-(methoxymethoxy)phenyl]pyridazin-4-yl}-4-phenylpiperidine-4-carboxylic acid (Intermediate 17) (41.00 mg, 0.0977 mmol) and tert-butyl 4-(methylamino)-1-azaspiro[5.5]undecane-1-carboxylate (82.81 mg, 0.2932 mmol) were dissolved in DMA (0.30 mL) and N,N-diisopropylethylamine (51.08 μL, 0.04 g, 0.2932 mmol), then [(dimethylamino)({[1,2,3]triazolo[4,5-b]pyridin-3-yloxy})methylidene]dimethylazanium; hexafluoro-lambda5-phosphanuide (44.60 mg, 0.1173 mmol) was added. Stirred at rt overnight. Partitioned between DCM and sat. sodium bicarbonate and then purified by normal phase silica gel chromatography (EtOAc/heptane) to yield tert-butyl 4-(N-methyl-1-{6-[2-(methoxymethoxy)phenyl]pyridazin-4-yl}-4-phenylpiperidine-4-amido)-1-azaspiro[5.5]undecane-1-carboxylate (0.062 g, 92.75%). LCMS: C₄₀H₅₃N₅O₅ requires: 683.4. found: m/z=684.8 [M+H]⁺.

### Step 2: Synthesis of N-{1-azaspiro[5.5]undecan-4-yl}-1-[6-(2-hydroxyphenyl)pyridazin-4-yl]-N-methyl-4-phenylpiperidine-4-carboxamide

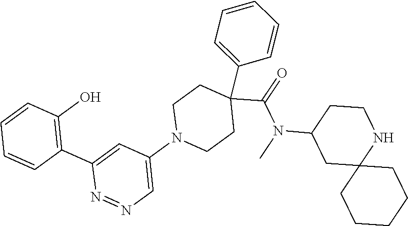 [SMILES: CN(C(=O)C1(c2ccccc2)CCN(c2cnnc(-c3ccccc3O)c2)CC1)C1CCNC2(CCCCC2)C1]

Dissolved tert-butyl 4-(N-methyl-1-{6-[2-(methoxymethoxy)phenyl]pyridazin-4-yl}-4-phenylpiperidine-4-amido)-1-azaspiro[5.5]undecane-1-carboxylate in 1:1 TFA/DCM (1 mL). After 2 h the reaction was concentrated down to an oil which was used in the next step without further purification. LCMS: C₃₃H₄₁N₅O₂ requires: 539.3. found: m/z=540.7 [M+H]⁺.

### Step 3: Synthesis of the Title Compound

This compound was synthesized according to step 2 of Example 98 using N-{1-azaspiro[5.5]undecan-4-yl}-1-[6-(2-hydroxyphenyl)pyridazin-4-yl]-N-methyl-4-phenylpiperidine-4-carboxamide. LCMS: C₅₀H₆₁N₇O₄ requires: 823.5. found: m/z=824.4 [M+H]⁺.

### Example 154

### N-{1-[(1-{4-[(3R)-2,6-dioxopiperidin-3-yl]phenyl}piperidin-4-yl)methyl]piperidin-4-yl}-1-[6-(2-hydroxyphenyl)pyridazin-4-yl]-N-methyl-4-(4-methyl-1H-pyrazol-1-yl)piperidine-4-carboxamide

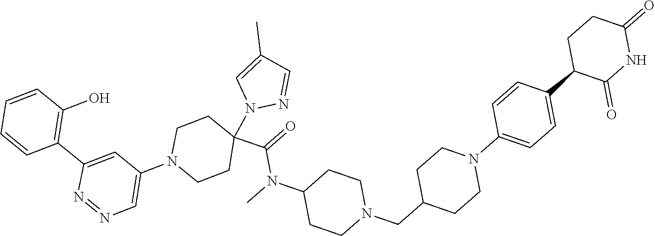 [SMILES: Cc1cnn(C2(C(=O)N(C)C3CCN(CC4CCN(c5ccc([C@H]6CCC(=O)NC6=O)cc5)CC4)CC3)CCN(c3cnnc(-c4ccccc4O)c3)CC2)c1]

### Step 1: Synthesis of 1-[6-(2-hydroxyphenyl)pyridazin-4-yl]-N-methyl-4-(4-methylpyrazol-1-yl)-N-(piperidin-4-yl)piperidine-4-carboxamide

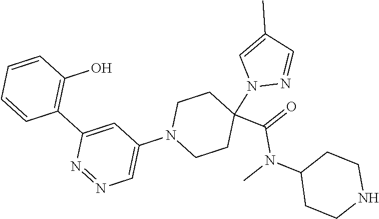 [SMILES: Cc1cnn(C2(C(=O)N(C)C3CCNCC3)CCN(c3cnnc(-c4ccccc4O)c3)CC2)c1]

To a solution of 1-[6-(2-hydroxyphenyl)pyridazin-4-yl]-4-(4-methylpyrazol-1-yl)piperidine-4-carboxylic acid (100.00 mg, 0.2636 mmol) in 1.6 mL DMA was added tert-butyl 4-(methylamino)piperidine-1-carboxylate (169.45 mg, 0.7907 mmol) and N,N-diisopropylethylamine (137.73 μL, 0.10 g, 0.7907 mmol). Cooled in an ice bath then [(dimethylamino)({[1,2,3]triazolo[4,5-b]pyridin-3-yloxy})methylidene]dimethylazanium; hexafluoro-lambda5-phosphanuide (150.32 mg, 0.3953 mmol) was added. Stirred at rt for 4 days. Diluted with EtOAc (6 mL) and washed with 1M citric acid (2×20 mL) and sodium bicarbonate (1×10 ml), then passed through a phase separator and concentrated to dryness to obtain 115 mg of crude. Added DCM (2 mL) and TFA (2 mL), then after 45 min, concentrated to dryness. Dissolved in 1:1 ACN/water (2 mL) and lyophilized. Used in the next step as is. LCMS: C₂₆H₃₃N₇O₂ requires: 475.3. found: m/z=476.2 [M+H]⁺.

### Step 2: Synthesis of the Title Compound

Combined 1-[6-(2-hydroxyphenyl)pyridazin-4-yl]-N-methyl-4-(4-methylpyrazol-1-yl)-N-(piperidin-4-yl)piperidine-4-carboxamide (38.00 mg, 0.0799 mmol) and 1-{4-[(3R)-2,6-dioxopiperidin-3-yl]phenyl}piperidine-4-carbaldehyde (26.40 mg, 0.0879 mmol) in DMA (0.20 mL) with N,N-diisopropylethylamine (69.59 μL, 0.05 g, 0.3995 mmol) and then added sodium triacetoxyborohydride (50.80 mg, 0.2397 mmol) and stirred for 1 h. Purified the solution directly by RP-HPLC to yield the title compound (0.0335 g, 55.17%). LCMS: C₄₃H₅₃N₉O₄ requires: 759.4, found: m/z=760.4 [M+H]^{+ 1}H NMR (500 MHz, DMSO) δ 10.77 (s, 1H), 8.95 (s, 1H), 8.14 (s, 1H), 8.11 (d, J=8.0 Hz, 1H), 7.73 (s, 1H), 7.55 (d, J=2.9 Hz, 1H), 7.40 (s, 1H), 7.34 (td, J=7.6, 1.6 Hz, 1H), 7.04 (d, J=8.3 Hz, 2H), 6.96-6.86 (m, 5H), 3.72 (dd, J=11.0, 4.9 Hz, 3H), 3.66 (s, 2H), 2.90 (s, 1H), 2.74 (s, 1H), 2.69-2.58 (m, 4H), 2.50-2.42 (m, 6H), 2.24 (s, 1H), 2.18-2.07 (m, 2H), 2.05-1.97 (m, 5H), 1.75 (s, 2H), 1.22 (s, 1H), 1.01 (s, 1H).

### Example 155

### (3R)-3-[4-(4-{[(3aRS,7aSR)-1-{1-[6-(2-hydroxyphenyl)pyridazin-4-yl]-4-phenylpiperidine-4-carbonyl}-octahydro-1H-pyrrolo[3,2-c]pyridin-5-yl]methyl}piperidin-1-yl)phenyl]piperidine-2,6-dione

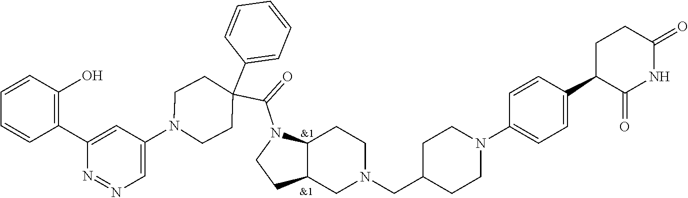 [SMILES: O=C1CC[C@H](c2ccc(N3CCC(CN4CC[C@H]5[C@H](CCN5C(=O)C5(c6ccccc6)CCN(c6cnnc(-c7ccccc7O)c6)CC5)C4)CC3)cc2)C(=O)N1]

### Step 1: Synthesis of 2-[5-(4-{octahydropyrrolo[3,2-c]pyridine-1-carbonyl}-4-phenylpiperidin-1-yl)pyridazin-3-yl]phenol

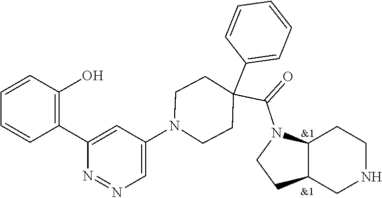 [SMILES: O=C(N1CC[C@@H]2CNCC[C@@H]21)C1(c2ccccc2)CCN(c2cnnc(-c3ccccc3O)c2)CC1]

1-[6-(2-hydroxyphenyl)pyridazin-4-yl]-4-phenylpiperidine-4-carboxylic acid (30.00 mg, 0.0799 mmol) and tert-butyl octahydropyrrolo[3,2-c]pyridine-5-carboxylate (21.70 mg, 0.0959 mmol) were dissolved in DMA (0.30 mL) and N,N-diisopropylethylamine (41.76 μL, 0.03 g, 0.2397 mmol), then [(dimethylamino)({[1,2,3]triazolo[4,5-b]pyridin-3-yloxy})methylidene]dimethylazanium; hexafluoro-lambda5-phosphanuide (36.46 mg, 0.0959 mmol) was added. Stirred at rt overnight. Partitioned between EtOAc and 1M citric acid, then washed with water and bicarb. Passed through a phase separator and concentrated to dryness. This material was dissolved in DCM (1 mL) and TFA (1 mL) and after 1 hour, it was concentrated by rotary evaporation, and then lyophilized twice from 1:1 ACN/water. Used in the next step as is without further purification. LCMS: C₂₉H₃₃N₅O₂ requires: 483.3. found: m/z=484.4 [M+H]⁺.

### Step 2: Synthesis of the Title Compound

Combined 2-[5-(4-{octahydropyrrolo[3,2-c]pyridine-1-carbonyl}-4-phenylpiperidin-1-yl)pyridazin-3-yl]phenol (20.10 mg, 0.0416 mmol) and 1-{4-[(3R)-2,6-dioxopiperidin-3-yl]phenyl}piperidine-4-carbaldehyde (13.73 mg, 0.0457 mmol) in DMA (0.20 mL) with N,N-diisopropylethylamine (36.20 μL, 0.03 g, 0.2078 mmol) and then added sodium triacetoxyborohydride (26.43 mg, 0.1247 mmol) and stirred for 1 h. Purified the solution directly by RP-HPLC to yield the title compound (0.0106 g, 33.21%). LCMS: C₄₆H₅₃N₇O₄ requires: 767.4, found: m/z=768.3 [M+H]⁺.

### Example 156

### N-[(3S)-2,6-dioxopiperidin-3-yl]-6-{4-[(4-{l-[6-(2-hydroxyphenyl)pyridazin-4-yl]-4-phenylpiperidine-4-amido}piperidin-1-yl)methyl]piperidin-1-yl}pyridine-3-carboxamide

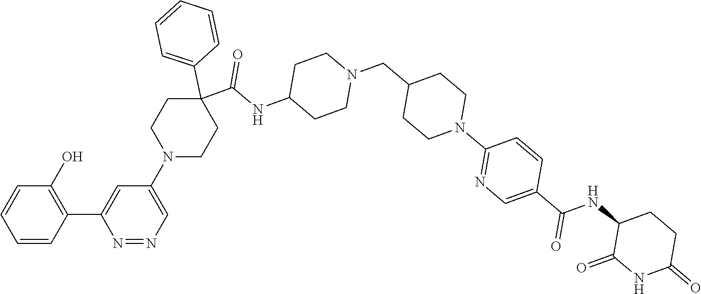 [SMILES: O=C1CC[C@H](NC(=O)c2ccc(N3CCC(CN4CCC(NC(=O)C5(c6ccccc6)CCN(c6cnnc(-c7ccccc7O)c6)CC5)CC4)CC3)nc2)C(=O)N1]

LCMS: C₄₄H₅₁N₉O₅ requires: 785.4. found: m/z=786.3 [M+H]⁺

### Example 157

### N-{1-[(1-{2-[(3S)-2,6-dioxopiperidin-3-yl]-1-oxo-1,2-dihydroisoquinolin-6-yl}piperidin-4-yl)methyl]piperidin-4-yl}-1-[6-(2-hydroxyphenyl)pyridazin-4-yl]-4-phenylpiperidine-4-carboxamide

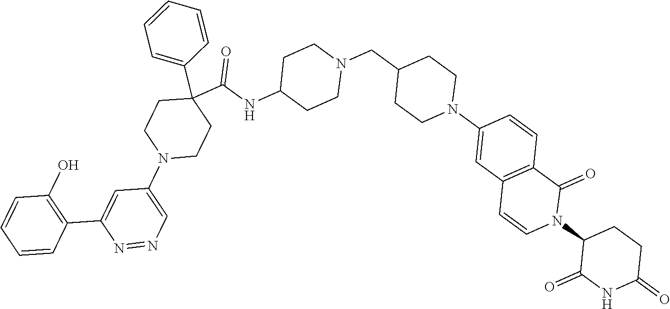 [SMILES: O=C1CC[C@H](n2ccc3cc(N4CCC(CN5CCC(NC(=O)C6(c7ccccc7)CCN(c7cnnc(-c8ccccc8O)c7)CC6)CC5)CC4)ccc3c2=O)C(=O)N1]

### Example 158

### rac-N-{1-[(1-{1-[(3R)-2,6-dioxopiperidin-3-yl]-3-methyl-2-oxo-2,3-dihydro-1H-1,3-benzodiazol-5-yl}piperidin-4-yl)methyl]piperidin-4-yl}-1-[6-(2-hydroxyphenyl)pyridazin-4-yl]-4-phenylpiperidine-4-carboxamide

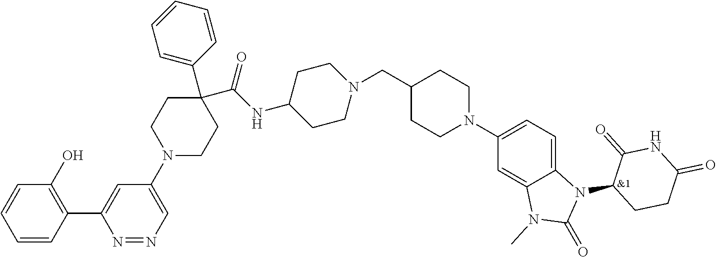 [SMILES: Cn1c(=O)n([C@@H]2CCC(=O)NC2=O)c2ccc(N3CCC(CN4CCC(NC(=O)C5(c6ccccc6)CCN(c6cnnc(-c7ccccc7O)c6)CC5)CC4)CC3)cc21]

### Example 159

### rac-(3R)-3-(5-{4-[(2-{1-[6-(2-hydroxyphenyl)pyridazin-4-yl]-4-phenylpiperidine-4-carbonyl}-2,7-diazaspiro[3.5]nonan-7-yl)methyl]piperidin-1-yl}-3-methyl-2-oxo-2,3-dihydro-1H-1,3-benzodiazol-1-yl)piperidine-2,6-dione

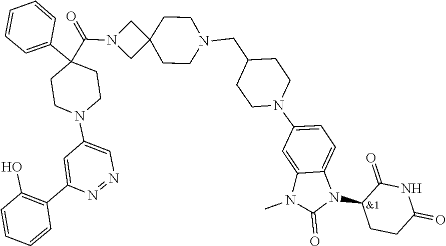 [SMILES: Cn1c(=O)n([C@@H]2CCC(=O)NC2=O)c2ccc(N3CCC(CN4CCC5(CC4)CN(C(=O)C4(c6ccccc6)CCN(c6cnnc(-c7ccccc7O)c6)CC4)C5)CC3)cc21]

### Example 160

### (3RS)-3-{4-[(3aS,6aS)-5-[(1-{1-[6-(2-hydroxyphenyl)pyridazin-4-yl]-4-phenylpiperidine-4-carbonyl}piperidin-4-yl)methyl]-octahydropyrrolo[3,4-c]pyrrol-2-yl]phenyl}piperidine-2,6-dione

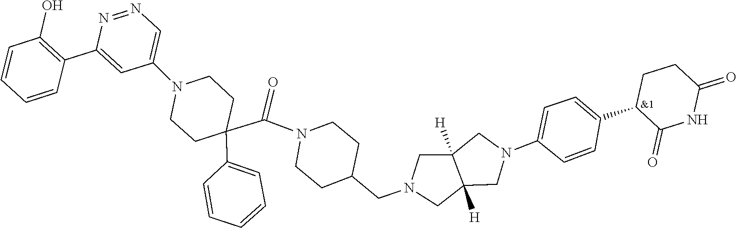 [SMILES: O=C1CC[C@@H](c2ccc(N3C[C@@H]4CN(CC5CCN(C(=O)C6(c7ccccc7)CCN(c7cnnc(-c8ccccc8O)c7)CC6)CC5)C[C@H]4C3)cc2)C(=O)N1]

LCMS: C₄₅H₅₁N₇O₄ requires: 753.4. found: m/z=754.4 [M+H]⁺

### Example 161

### N-{5-[(1-{4-[(3R)-2,6-dioxopiperidin-3-yl]phenyl}piperidin-4-yl)methyl]-5-azaspiro[3.5]nonan-8-yl}-1-[6-(2-hydroxyphenyl)pyridazin-4-yl]-4-(oxan-4-yl)piperidine-4-carboxamide

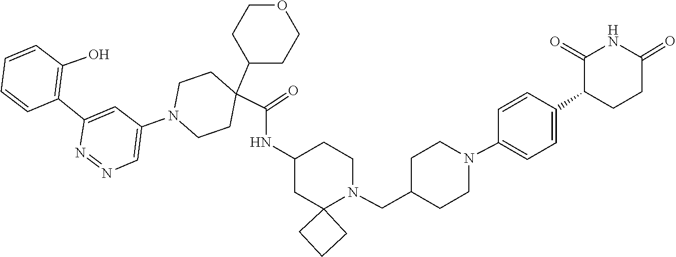 [SMILES: O=C1CC[C@H](c2ccc(N3CCC(CN4CCC(NC(=O)C5(C6CCOCC6)CCN(c6cnnc(-c7ccccc7O)c6)CC5)CC45CCC5)CC3)cc2)C(=O)N1]

### Step 1: Synthesis of N-{5-azaspiro[3.5]nonan-8-yl}-1-[6-(2-hydroxyphenyl)pyridazin-4-yl]-4-(oxan-4-yl)piperidine-4-carboxamide

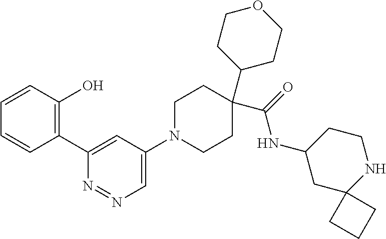 [SMILES: O=C(NC1CCNC2(CCC2)C1)C1(C2CCOCC2)CCN(c2cnnc(-c3ccccc3O)c2)CC1]

A solution of 1-[6-(2-hydroxyphenyl)pyridazin-4-yl]-4-(oxan-4-yl)piperidine-4-carboxylic acid (14.20 mg, 0.0370 mmol) in 0.2 mL DMA was added to tert-butyl 8-amino-5-azaspiro[3.5]nonane-5-carboxylate (9.79 mg, 0.0407 mmol) with N,N-diisopropylethylamine (19.35 μL, 0.01 g, 0.1111 mmol) and then [(dimethylamino)({[1,2,3]triazolo[4,5-b]pyridin-3-yloxy})methylidene]dimethylazanium; hexafluoro-lambda5-phosphanuide (16.90 mg, 0.0444 mmol) was added as a solution in 0.2 mL DMA. Stirred at rt. Diluted with DCM (3 mL) and washed with 1M citric acid (2×4 mL) and bicarb (1×4 ml), then passed through a phase separator. Added TFA (2 mL), then after 45 min, concentrated to dryness. Dissolved in 1:1 ACN/water (2 mL) and lyophilized. Used in the next step as is. LCMS: C₂₉H₃₉N₅O₃ requires: 505.3. found: m/z=506.7 [M+H]⁺.

The title compound was synthesized according to step 2 of Example 98 using N-{5-azaspiro[3.5]nonan-8-yl}-1-[6-(2-hydroxyphenyl)pyridazin-4-yl]-4-(oxan-4-yl)piperidine-4-carboxamide LCMS: C₄₆H₅₉N₇O₅ requires: 789.5. found: m/z=790.3 [M+H]⁺.

### Example 162

### N-{5-[(1-{4-[(3R)-2,6-dioxopiperidin-3-yl]phenyl}piperidin-4-yl)methyl]-5-azaspiro[3.5]nonan-8-yl}-1-[6-(2-hydroxyphenyl)pyridazin-4-yl]-4-(3-methyl-1H-pyrazol-1-yl)piperidine-4-carboxamide

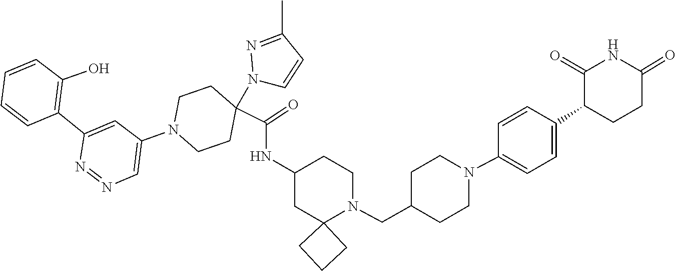 [SMILES: Cc1ccn(C2(C(=O)NC3CCN(CC4CCN(c5ccc([C@H]6CCC(=O)NC6=O)cc5)CC4)C4(CCC4)C3)CCN(c3cnnc(-c4ccccc4O)c3)CC2)n1]

### Step 1: Synthesis of N-{5-azaspiro[3.5]nonan-8-yl}-1-[6-(2-hydroxyphenyl)pyridazin-4-yl]-4-(3-methylpyrazol-1-yl)piperidine-4-carboxamide

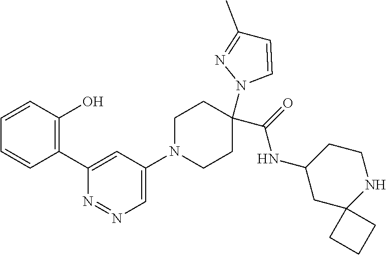 [SMILES: Cc1ccn(C2(C(=O)NC3CCNC4(CCC4)C3)CCN(c3cnnc(-c4ccccc4O)c3)CC2)n1]

A solution of 1-[6-(2-hydroxyphenyl)pyridazin-4-yl]-4-(3-methylpyrazol-1-yl)piperidine-4-carboxylic acid (Intermediate 5) (14.01 mg, 0.0369 mmol) in 0.2 mL DMA was added to tert-butyl 8-amino-5-azaspiro[3.5]nonane-5-carboxylate (9.76 mg, 0.0406 mmol) with N,N-diisopropylethylamine (19.30 μL, 0.01 g, 0.1108 mmol) and then [(dimethylamino)({[1,2,3]triazolo[4,5-b]pyridin-3-yloxy})methylidene]dimethylazanium; hexafluoro-lambda5-phosphanuide (16.85 mg, 0.0443 mmol) was added as a solution in 0.2 mL DMA. Stirred at rt. Diluted with DCM (3 mL) and washed with 1M citric acid (2×4 mL) and bicarb (1×4 ml), then passed through a phase separator. Added TFA (2 mL), then after 45 min, concentrated to dryness. Dissolved in 1:1 ACN/water (2 mL) and lyophilized. Used in the next step as is. LCMS: C₂₈H₃₅N₇O₂ requires: 501.3. found: m/z=502.7 [M+H]⁺.

### Step 2: Synthesis of the Title Compound

This compound was synthesized according to step 2 of Example 98 using N-{5-azaspiro[3.5]nonan-8-yl}-1-[6-(2-hydroxyphenyl)pyridazin-4-yl]-4-(3-methylpyrazol-1-yl)piperidine-4-carboxamide. C₄₅H₅₅N₉O₄ requires: 785.4. found: m/z=786.3 [M+H]⁺.

### Example 163

### rac-(3R)-3-(4-{2-[(1-{1-[6-(2-hydroxyphenyl)pyridazin-4-yl]-4-phenylpiperidine-4-carbonyl}piperidin-4-yl)methyl]-2,8-diazaspiro[4.5]decan-8-yl}phenyl)piperidine-2,6-dione

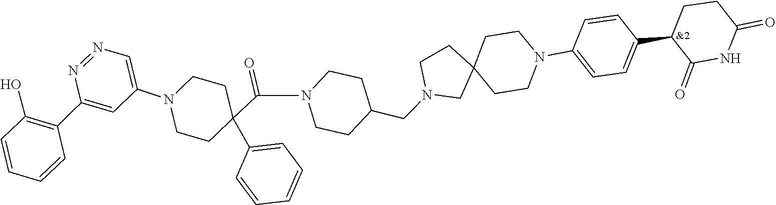 [SMILES: O=C1CC[C@H](c2ccc(N3CCC4(CCN(CC5CCN(C(=O)C6(c7ccccc7)CCN(c7cnnc(-c8ccccc8O)c7)CC6)CC5)C4)CC3)cc2)C(=O)N1]

LCMS: C₄₇H₅₅N₇O₄ requires: 781.4. found: m/z=782.4 [M+H]⁺

### Example 164

### (3R)-3-(4-{4-[2-({1-[6-(2-hydroxyphenyl)pyridazin-4-yl]-4-phenylpiperidin-4-yl}methyl)-2,7-diazaspiro[3.5]nonane-7-carbonyl]piperidin-1-yl}phenyl)piperidine-2,6-dione

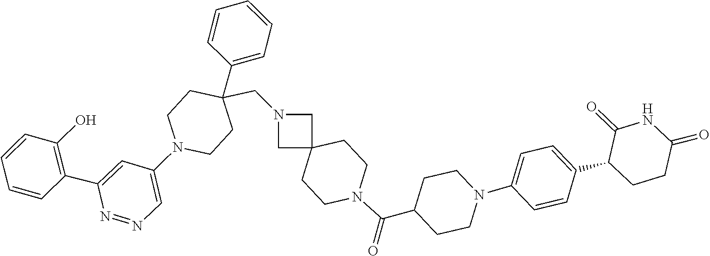 [SMILES: O=C1CC[C@H](c2ccc(N3CCC(C(=O)N4CCC5(CC4)CN(CC4(c6ccccc6)CCN(c6cnnc(-c7ccccc7O)c6)CC4)C5)CC3)cc2)C(=O)N1]

### Step 1: Synthesis of 2-[5-(4-{2,7-diazaspiro[3.5]nonan-2-ylmethyl}-4-phenylpiperidin-1-yl)pyridazin-3-yl]phenol

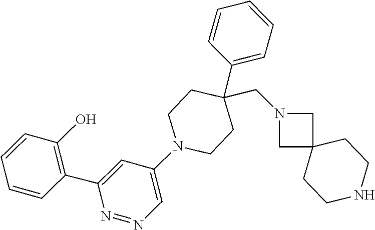 [SMILES: Oc1ccccc1-c1cc(N2CCC(CN3CC4(CCNCC4)C3)(c3ccccc3)CC2)cnn1]

Combined 1-[6-(2-hydroxyphenyl)pyridazin-4-yl]-4-phenylpiperidine-4-carbaldehyde (10.00 mg, 0.0278 mmol) and tert-butyl 2,7-diazaspiro[3.5]nonane-7-carboxylate (6.93 mg, 0.0306 mmol) in DMA (0.30 mL) and then added sodium triacetoxyborohydride (17.69 mg, 0.0835 mmol) and stirred overnight. Added another portion of STAB and stirred overnight. Diluted with DCM (1 mL) and washed with water (2 mL), then sat bicarb (2 mL). Passed through a phase separator. Added TFA (1 mL), then after 45 min, concentrated to dryness. Dissolved in 1:1 ACN/water (2 mL) and lyophilized. Used in the next step as is. LCMS: C₂₉H₃₅N₅₀ requires: 469.3. found: m/z=470.4 [M+H]⁺.

### Step 2: Synthesis of the Title Compound

Combined a solution of 2-[5-(4-{2,7-diazaspiro[3.5]nonan-2-ylmethyl}-4-phenylpiperidin-1-yl)pyridazin-3-yl]phenol (4.10 mg, 0.0093 mmol) in 100 μL DMA, with N,N-diisopropylethylamine (9.73 μL, 0.01 g, 0.0557 mmol) and a solution of 1-{4-[(3R)-2,6-dioxopiperidin-3-yl]phenyl}piperidine-4-carboxylic acid (2.94 mg, 0.0093 mmol) in 100 μL DMA and then added a solution of [(dimethylamino)({[1,2,3]triazolo[4,5-b]pyridin-3-yloxy})methylidene]dimethylazanium; hexafluoro-lambda5-phosphanuide (3.88 mg, 0.0102 mmol) in 100 μL DMA and stirred at rt. Purified the solution directly by RP-HPLC to yield the title compound (0.0034 g, 47.30%) LCMS: C₄₆H₅₃N₇O₄ requires: 767.4. found: m/z=768.4 [M+H]⁺.

### Example 165

### (3RS&)-3-[4-(4-{[(3aRS,6aSR)-5-{1-[6-(2-hydroxyphenyl)pyridazin-4-yl]-4-phenylpiperidine-4-carbonyl}-3aH,4H,5H,6H,6aH-pyrrolo[3,4-d][1,2]oxazol-3-yl]methyl}piperazin-1-yl)phenyl]piperidine-2,6-dione

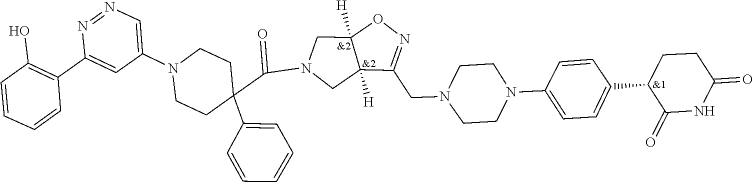 [SMILES: O=C1CC[C@@H](c2ccc(N3CCN(CC4=NO[C@@H]5CN(C(=O)C6(c7ccccc7)CCN(c7cnnc(-c8ccccc8O)c7)CC6)C[C@H]45)CC3)cc2)C(=O)N1]

LCMS: C₄₃H₄₆N₈O₅ requires: 754.4. found: m/z=755.3 [M+H]⁺

### Example 166

### (3RS)-3-(4-{[(1r,4r)-4-[(6-{1-[6-(2-hydroxyphenyl)pyridazin-4-yl]-4-phenylpiperidine-4-carbonyl}-2,6-diazaspiro[3.3]heptan-2-yl)methyl]cyclohexyl]oxy}phenyl)piperidine-2,6-dione

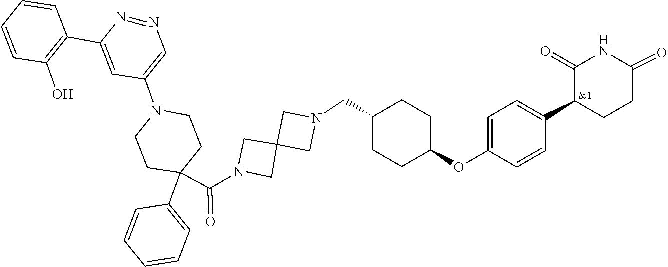 [SMILES: O=C1CC[C@@H](c2ccc(O[C@H]3CC[C@H](CN4CC5(C4)CN(C(=O)C4(c6ccccc6)CCN(c6cnnc(-c7ccccc7O)c6)CC4)C5)CC3)cc2)C(=O)N1]

Combined 2-[5-(4-{2,6-diazaspiro[3.3]heptane-2-carbonyl}-4-phenylpiperidin-1-yl)pyridazin-3-yl]phenol; tris(trifluoroacetic acid) (8.01 mg, 0.0100 mmol) and (1r,4r)-4-{4-[(3RS)-2,6-dioxopiperidin-3-yl]phenoxy}cyclohexane-1-carbaldehyde (3.48 mg, 0.0110 mmol) in DMA (0.20 mL) with N,N-diisopropylethylamine (8.75 μL, 0.01 g, 0.0502 mmol) and then added sodium triacetoxyborohydride (6.39 mg, 0.0301 mmol) and stirred overnight. Purified the solution directly by RP-HPLC to yield the title compound (0.0046 g, 60.25%). LCMS: C₄₅H₅₀N₆O₅ requires: 754.4, found: m/z=755.4 [M+H]⁺.

### Example 167

### rac-(3R)-3-(4-{4-[2-(6-{1-[6-(2-hydroxyphenyl)pyridazin-4-yl]-4-phenylpiperidine-4-carbonyl}-2,6-diazaspiro[3.5]nonan-2-yl)ethyl]piperidin-1-yl}phenyl)piperidine-2,6-dione

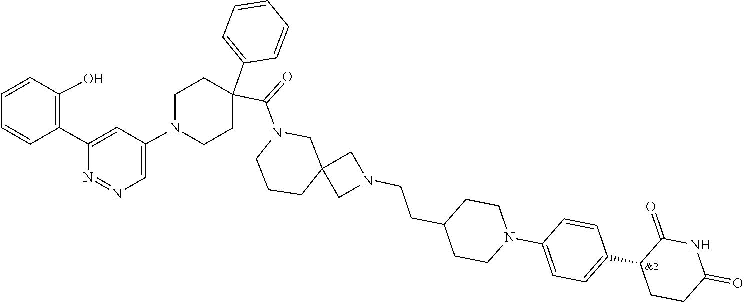 [SMILES: O=C1CC[C@H](c2ccc(N3CCC(CCN4CC5(CCCN(C(=O)C6(c7ccccc7)CCN(c7cnnc(-c8ccccc8O)c7)CC6)C5)C4)CC3)cc2)C(=O)N1]

### Step 1: Synthesis of 2-[5-(4-{2,6-diazaspiro[3.5]nonane-6-carbonyl}-4-phenylpiperidin-1-yl)pyridazin-3-yl]phenol

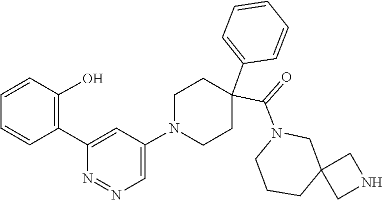 [SMILES: O=C(N1CCCC2(CNC2)C1)C1(c2ccccc2)CCN(c2cnnc(-c3ccccc3O)c2)CC1]

This intermediate was synthesized according to step 1 of Example 106 using 1-[6-(2-hydroxyphenyl)pyridazin-4-yl]-4-phenylpiperidine-4-carboxylic acid and tert-butyl 2,6-diazaspiro[3.5]nonane-2-carboxylate. LCMS: C₂₉H₃₃N₅O₂ requires: 483.3. found: m/z=484.6 [M+H]⁺.

### Step 2: Synthesis of the Title Compound

This compound was synthesis according to step 2 of example 98 using 2-[5-(4-{2,6-diazaspiro[3.5]nonane-6-carbonyl}-4-phenylpiperidin-1-yl)pyridazin-3-yl]phenol and 2-{1-[4-(2,6-dioxopiperidin-3-yl)phenyl]piperidin-4-yl}acetaldehyde. LCMS: C₄₇H₅₅N₇O₄ requires: 781.4, found: m/z=782.3 [M+H]⁺.

### Example 168

### N-[(7RS)-4-[(1-{4-[(3R)-2,6-dioxopiperidin-3-yl]phenyl}piperidin-4-yl)methyl]-4-azaspiro[2.5]octan-7-yl]-1-[6-(2-hydroxyphenyl)pyridazin-4-yl]-N-methyl-4-phenoxypiperidine-4-carboxamide

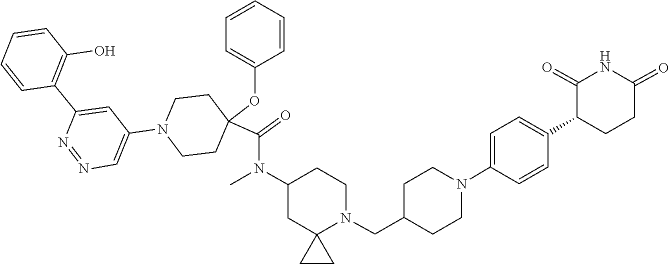 [SMILES: CN(C(=O)C1(Oc2ccccc2)CCN(c2cnnc(-c3ccccc3O)c2)CC1)C1CCN(CC2CCN(c3ccc([C@H]4CCC(=O)NC4=O)cc3)CC2)C2(CC2)C1]

### Step 1: Synthesis of N-{4-azaspiro[2.5]octan-7-yl}-1-[6-(2-hydroxyphenyl)pyridazin-4-yl]-N-methyl-4-phenoxypiperidine-4-carboxamide

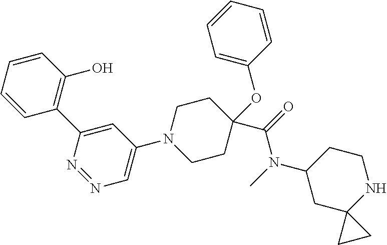 [SMILES: CN(C(=O)C1(Oc2ccccc2)CCN(c2cnnc(-c3ccccc3O)c2)CC1)C1CCNC2(CC2)C1]

This intermediate was synthesized according to step 1 of Example 172 using tert-butyl 7-(methylamino)-4-azaspiro[2.5]octane-4-carboxylate. LCMS: C₃₀H₃₅N₅O₃ requires: 513.3. found: m/z=514.7 [M+H]⁺.

### Step 2: Synthesis of the Title Compound

This compound was synthesized according to step 2 of Example 186 using N-{4-azaspiro[2.5]octan-7-yl}-1-[6-(2-hydroxyphenyl)pyridazin-4-yl]-N-methyl-4-phenoxypiperidine-4-carboxamide and 1-{4-[(3R)-2,6-dioxopiperidin-3-yl]phenyl}piperidine-4-carbaldehyde. LCMS: C₄₇H₅₅N₇O₅ requires: 797.4. found: m/z=798.4 [M+H]⁺.

### Example 169

### N-{5-[(1-{4-[(3R)-2,6-dioxopiperidin-3-yl]phenyl}piperidin-4-yl)methyl]-5-azaspiro[3.5]nonan-8-yl}-1-[6-(2-hydroxyphenyl)pyridazin-4-yl]-4-(3-methoxyphenyl)piperidine-4-carboxamide

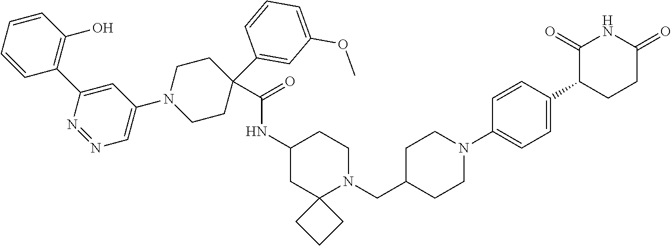 [SMILES: COc1cccc(C2(C(=O)NC3CCN(CC4CCN(c5ccc([C@H]6CCC(=O)NC6=O)cc5)CC4)C4(CCC4)C3)CCN(c3cnnc(-c4ccccc4O)c3)CC2)c1]

### Synthesis of the title compound

Combined a solution of N-{5-azaspiro[3.5]nonan-8-yl}-1-[6-(2-hydroxyphenyl)pyridazin-4-yl]-4-(3-methoxyphenyl)piperidine-4-carboxamide (6.40 mg, 0.0125 mmol) in 0.1 mL DMA to a solution of 1-{4-[(3R)-2,6-dioxopiperidin-3-yl]phenyl}piperidine-4-carbaldehyde (4.12 mg, 0.0137 mmol) in 0.1 mL DMA with N,N-diisopropylethylamine (13.02 μL, 0.01 g, 0.0748 mmol) and then added a solution of sodium triacetoxyborohydride (7.92 mg, 0.0374 mmol) in 0.1 mL DMA and stirred overnight. Purified the solution directly by RP-HPLC to yield the title compound (0.0012 g, 11.86%). LCMS: C₄₈H₅₇N₇O₅ requires: 811.4. found: m/z=812.4 [M+H]⁺.

### Example 170

### rac-N-{2-[4-(4-{[(3R)-2,6-dioxopiperidin-3-yl]amino}phenyl)piperidin-1-yl]ethyl}-1-[6-(2-hydroxyphenyl)pyridazin-4-yl]-4-phenylpiperidine-4-carboxamide

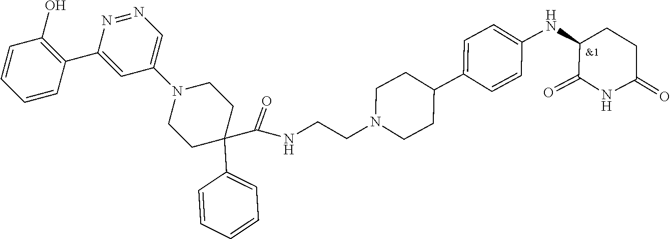 [SMILES: O=C1CC[C@H](Nc2ccc(C3CCN(CCNC(=O)C4(c5ccccc5)CCN(c5cnnc(-c6ccccc6O)c5)CC4)CC3)cc2)C(=O)N1]

LCMS: C₄₀H₄₅N₇O₄ requires: 687.4. found: m/z=688.3 [M+H]⁺

### Example 171

### rac-1-[(4-{4-[(3R)-2,6-dioxopiperidin-3-yl]phenyl}piperazin-1-yl)methyl]-6-{1-[6-(2-hydroxyphenyl)pyridazin-4-yl]-4-phenylpiperidine-4-carbonyl}-6-azaspiro[2.5]octane-1-carbonitrile

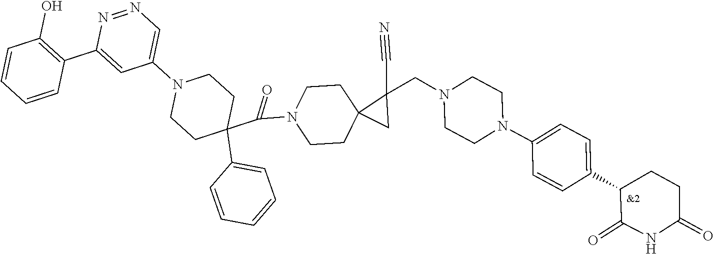 [SMILES: N#CC1(CN2CCN(c3ccc([C@@H]4CCC(=O)NC4=O)cc3)CC2)CC12CCN(C(=O)C1(c3ccccc3)CCN(c3cnnc(-c4ccccc4O)c3)CC1)CC2]

LCMS: C₄₆H₅₀N₈O₄ requires: 778.4. found: m/z=779.4 [M+H]⁺

### Example 172

### N-(2-{4-[2-(4-{4-[(3R)-2,6-dioxopiperidin-3-yl]phenyl}piperazin-1-yl)acetyl]piperazin-1-yl}ethyl)-1-[6-(2-hydroxyphenyl)pyridazin-4-yl]-4-phenoxypiperidine-4-carboxamide

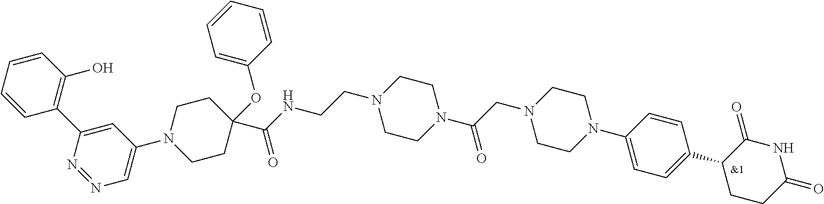 [SMILES: O=C1CC[C@H](c2ccc(N3CCN(CC(=O)N4CCN(CCNC(=O)C5(Oc6ccccc6)CCN(c6cnnc(-c7ccccc7O)c6)CC5)CC4)CC3)cc2)C(=O)N1]

### Step 1: Synthesis of 1-[6-(2-hydroxyphenyl)pyridazin-4-yl]-4-phenoxy-N-[2-(piperazin-1-yl)ethyl]piperidine-4-carboxamide

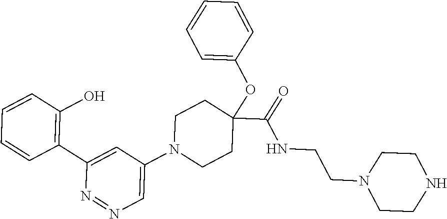 [SMILES: O=C(NCCN1CCNCC1)C1(Oc2ccccc2)CCN(c2cnnc(-c3ccccc3O)c2)CC1]

A solution of 1-[6-(2-hydroxyphenyl)pyridazin-4-yl]-4-phenoxypiperidine-4-carboxylic acid (15.00 mg, 0.0383 mmol) in 0.2 mL DMA was added to tert-butyl 4-(2-aminoethyl)piperazine-1-carboxylate (10.55 mg, 0.0460 mmol) with N,N-diisopropylethylamine (20.03 μL, 0.01 g, 0.1150 mmol) and then [(dimethylamino)({[1,2,3]triazolo[4,5-b]pyridin-3-yloxy})methylidene]dimethylazanium; hexafluoro-lambda5-phosphanuide (17.49 mg, 0.0460 mmol) was added as a solution in 0.2 mL DMA. Stirred at rt. Diluted with DCM (3 mL) and washed with 1M citric acid (2×4 mL) and bicarb (1×4 ml), then passed through a phase separator. Added TFA (2 mL), then after 45 min, concentrated to dryness. Dissolved in 1:1 ACN/water (2 mL) and lyophilized. Used in the next step as is. LCMS: C₂₉H₃₉N₅O₃ requires: 502.3. found: m/z=503.6 [M+H]⁺.

### Step 2: Synthesis of the Title Compound

Combined a solution of 1-[6-(2-hydroxyphenyl)pyridazin-4-yl]-4-phenoxy-N-[2-(piperazin-1-yl)ethyl]piperidine-4-carboxamide (6.40 mg, 0.0127 mmol) in 100 μL DMA, with N,N-diisopropylethylamine (13.34 μL, 0.01 g, 0.0764 mmol) and a solution of {4-[4-(2,6-dioxopiperidin-3-yl)phenyl]piperazin-1-yl}acetic acid (4.40 mg, 0.0140 mmol) in 100 μL DMA and then added a solution of [(dimethylamino)({[1,2,3]triazolo[4,5-b]pyridin-3-yloxy})methylidene]dimethylazanium; hexafluoro-lambda5-phosphanuide (5.33 mg, 0.0140 mmol) in 100 μL DMA and stirred at rt. Purified the solution directly by RP-HPLC to yield the title compound (0.0004 g, 3.68%). LCMS: C₄₅H₅₃N₉O₆ requires: 815.4. found: m/z=816.2 [M+H]⁺.

### Example 173

### 1-[6-(2-hydroxyphenyl)pyridazin-4-yl]-4-phenoxy-N-[(1r,4r)-4-[2-(4-{4-[(3R)-2,6-dioxopiperidin-3-yl]phenyl}piperazin-1-yl)-N-methylacetamido]cyclohexyl]piperidine-4-carboxamide

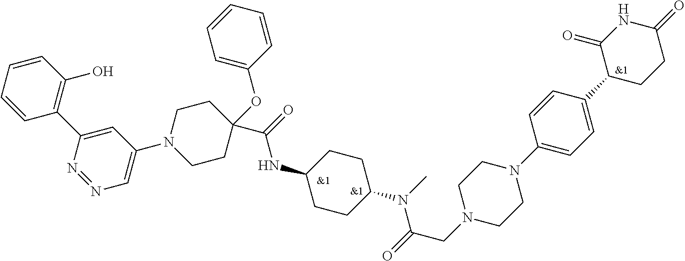 [SMILES: CN(C(=O)CN1CCN(c2ccc([C@H]3CCC(=O)NC3=O)cc2)CC1)[C@H]1CC[C@H](NC(=O)C2(Oc3ccccc3)CCN(c3cnnc(-c4ccccc4O)c3)CC2)CC1]

### Step 1: Synthesis of 1-[6-(2-hydroxyphenyl)pyridazin-4-yl]-4-phenoxy-N-[(1r,4r)-4-(methylamino)cyclohexyl]piperidine-4-carboxamide

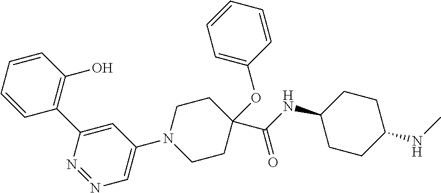 [SMILES: CN[C@H]1CC[C@H](NC(=O)C2(Oc3ccccc3)CCN(c3cnnc(-c4ccccc4O)c3)CC2)CC1]

This intermediate was synthesized according to step 1 of Example 172 using tert-butyl N-methyl-N-[(1r,4r)-4-aminocyclohexyl]carbamate. LCMS: C₂₉H₃₉N₅O₃ requires: 501.3. found: m/z=502.7 [M+H]⁺.

### Step 2: Synthesis of the Title Compound

This compound was synthesized according to step 2 of Example 172 using 1-[6-(2-hydroxyphenyl)pyridazin-4-yl]-4-phenoxy-N-[(1r,4r)-4-(methylamino)cyclohexyl]piperidine-4-carboxamide. LCMS: C₄₆H₅₄N₈O₆ requires: 814.4. found: m/z=815.3 [M+H]⁺.

### Example 174

### N-(1-{1-[2-(1-{4-[(3R)-2,6-dioxopiperidin-3-yl]phenyl}piperidin-4-yl)ethyl]piperidin-4-yl}-1H-pyrazol-4-yl)-1-[6-(2-hydroxyphenyl)pyridazin-4-yl]-4-phenoxypiperidine-4-carboxamide

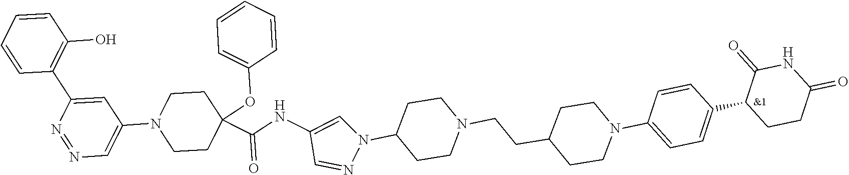 [SMILES: O=C1CC[C@H](c2ccc(N3CCC(CCN4CCC(n5cc(NC(=O)C6(Oc7ccccc7)CCN(c7cnnc(-c8ccccc8O)c7)CC6)cn5)CC4)CC3)cc2)C(=O)N1]

### Step 1: Synthesis of 1-[6-(2-hydroxyphenyl)pyridazin-4-yl]-4-phenoxy-N-[1-(piperidin-4-yl)pyrazol-4-yl]piperidine-4-carboxamide

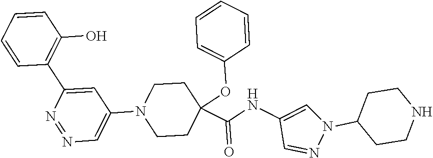 [SMILES: O=C(Nc1cnn(C2CCNCC2)c1)C1(Oc2ccccc2)CCN(c2cnnc(-c3ccccc3O)c2)CC1]

This intermediate was synthesized according to step 1 of Example 172 using tert-butyl 4-(4-aminopyrazol-1-yl)piperidine-1-carboxylate. LCMS: C₃₀H₃₃N₇03 requires: 539.3. found: m/z=540.6 [M+H]⁺.

### Step 2: Synthesis of the Title Compound

This compound was synthesized according to step 2 of Example 186 using 1-[6-(2-hydroxyphenyl)pyridazin-4-yl]-4-phenoxy-N-[1-(piperidin-4-yl)pyrazol-4-yl]piperidine-4-carboxamide. LCMS: C₄₈H₅₅N₉O₅ requires: 837.4. found: m/z=838.4 [M+H]⁺.

### Example 175

### 1-[6-(2-hydroxyphenyl)pyridazin-4-yl]-4-phenyl-N-[(1r,4r)-4-{[(1-{4-[(3RS)-2,6-dioxopiperidin-3-yl]phenyl}piperidin-4-yl)methyl]amino}cyclohexyl]piperidine-4-carboxamide

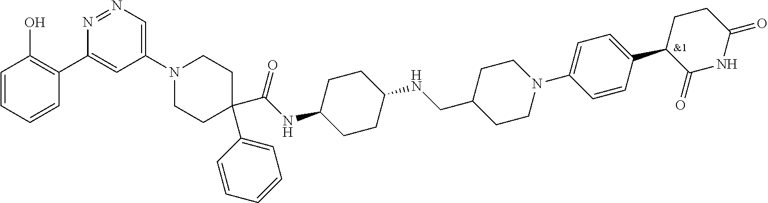 [SMILES: O=C1CC[C@H](c2ccc(N3CCC(CN[C@H]4CC[C@H](NC(=O)C5(c6ccccc6)CCN(c6cnnc(-c7ccccc7O)c6)CC5)CC4)CC3)cc2)C(=O)N1]

LCMS: C₄₅H₅₃N₇O₄ requires: 755.4. found: m/z=756.3 [M+H]⁺

### Example 176

### rac-(3R)-3-(4-{1-[(1-{1-[6-(2-hydroxyphenyl)pyridazin-4-yl]-4-phenylpiperidine-4-carbonyl}-3-methylpyrrolidin-3-yl)methyl]piperidin-4-yl}phenyl)piperidine-2,6-dione

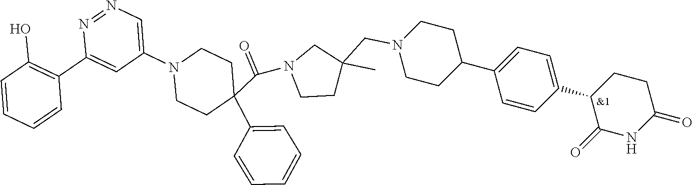 [SMILES: CC1(CN2CCC(c3ccc([C@@H]4CCC(=O)NC4=O)cc3)CC2)CCN(C(=O)C2(c3ccccc3)CCN(c3cnnc(-c4ccccc4O)c3)CC2)C1]

LCMS: C₄₄H₅₀N₆O₄ requires: 726.4. found: m/z=727.3 [M+H]⁺

### Example 177

### rac-(3R)-3-[(4-{1-[(2-{1-[6-(2-hydroxyphenyl)pyridazin-4-yl]-4-phenylpiperidine-4-carbonyl}-2-azaspiro[3.3]heptan-5-yl)methyl]piperidin-4-yl}phenyl)amino]piperidine-2,6-dione

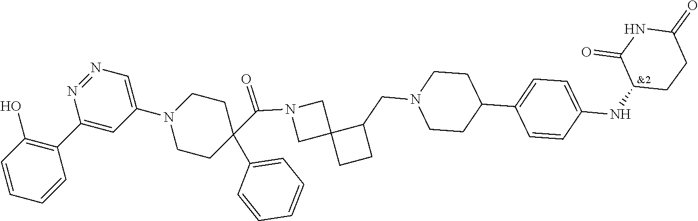 [SMILES: O=C1CC[C@H](Nc2ccc(C3CCN(CC4CCC45CN(C(=O)C4(c6ccccc6)CCN(c6cnnc(-c7ccccc7O)c6)CC4)C5)CC3)cc2)C(=O)N1]

LCMS: C₄₅H₅₁N₇O₄ requires: 753.4. found: m/z=754.4 [M+H]⁺

### Example 178

### 1-[6-(2-hydroxyphenyl)pyridazin-4-yl]-4-phenyl-N-[(1r,4r)-4-[(8-{4-[(3RS)-2,6-dioxopiperidin-3-yl]phenyl}-1-oxa-8-azaspiro[4.5]decan-3-yl)amino]cyclohexyl]piperidine-4-carboxamide

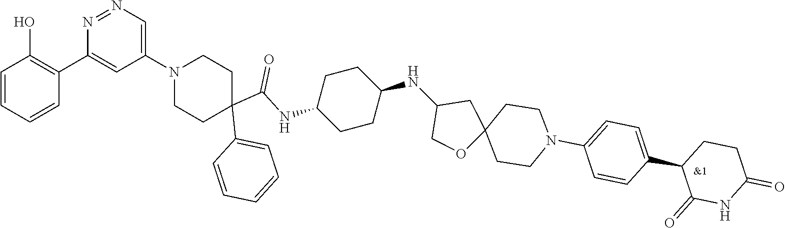 [SMILES: O=C1CC[C@H](c2ccc(N3CCC4(CC3)CC(N[C@H]3CC[C@H](NC(=O)C5(c6ccccc6)CCN(c6cnnc(-c7ccccc7O)c6)CC5)CC3)CO4)cc2)C(=O)N1]

LCMS: C₄₇H₅₅N₇O₅ requires: 797.4. found: m/z=798.4 [M+H]⁺

### Example 179

### rac-N-{1-[(1-{4-[(3R)-2,6-dioxopiperidin-3-yl]phenyl}-4-fluoropiperidin-4-yl)methyl]piperidin-4-yl}-1-[6-(2-hydroxyphenyl)pyridazin-4-yl]-4-phenylpiperidine-4-carboxamide

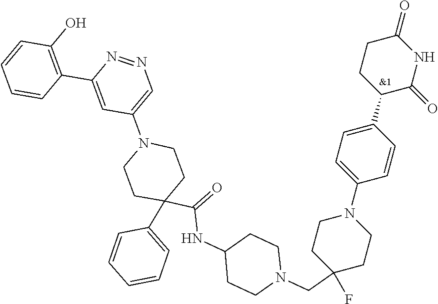 [SMILES: O=C1CC[C@@H](c2ccc(N3CCC(F)(CN4CCC(NC(=O)C5(c6ccccc6)CCN(c6cnnc(-c7ccccc7O)c6)CC5)CC4)CC3)cc2)C(=O)N1]

LCMS: C₄₄H₅₀FN₇O₄ requires: 759.4. found: m/z=760.4 [M+H]⁺

### Example 180

### (3R)-3-[4-(4-{[(1-{1-[6-(2-hydroxyphenyl)pyridazin-4-yl]-4-phenoxypiperidine-4-carbonyl}piperidin-4-yl)amino]methyl}piperidin-1-yl)phenyl]piperidine-2,6-dione

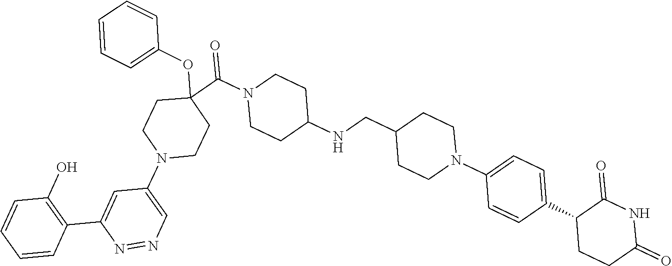 [SMILES: O=C1CC[C@H](c2ccc(N3CCC(CNC4CCN(C(=O)C5(Oc6ccccc6)CCN(c6cnnc(-c7ccccc7O)c6)CC5)CC4)CC3)cc2)C(=O)N1]

### Step 1: Synthesis of 2-{5-[4-(4-aminopiperidine-1-carbonyl)-4-phenoxypiperidin-1-yl]pyridazin-3-yl}phenol

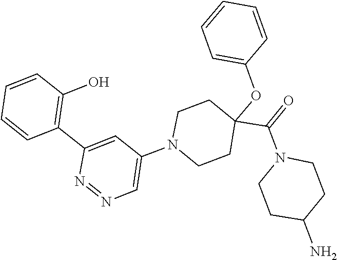 [SMILES: NC1CCN(C(=O)C2(Oc3ccccc3)CCN(c3cnnc(-c4ccccc4O)c3)CC2)CC1]

This intermediate was synthesized according to step 1 of Example 172 using tert-butyl N-(piperidin-4-yl)carbamate. LCMS: C₂₇H₃₁N₅O₃ requires: 473.2. found: m/z=474.6 [M+H]⁺.

### Step 2: Synthesis of the Title Compound

This compound was synthesized according to step 2 of Example 186 using 2-{5-[4-(4-aminopiperidine-1-carbonyl)-4-phenoxypiperidin-1-yl]pyridazin-3-yl}phenol and 1-{4-[(3R)-2,6-dioxopiperidin-3-yl]phenyl}piperidine-4-carbaldehyde. LCMS: C₄₄H₅₁N₇O₅ requires: 757.4, found: m/z=758.6 [M+H]⁺.

### Example 181

### (3RS)-3-{6-[(3S)-3-[2-(6-{1-[6-(2-hydroxyphenyl)pyridazin-4-yl]-4-phenylpiperidine-4-carbonyl}-2,6-diazaspiro[3.3]heptan-2-yl)ethyl]pyrrolidin-1-yl]pyridin-3-yl}piperidine-2,6-dione

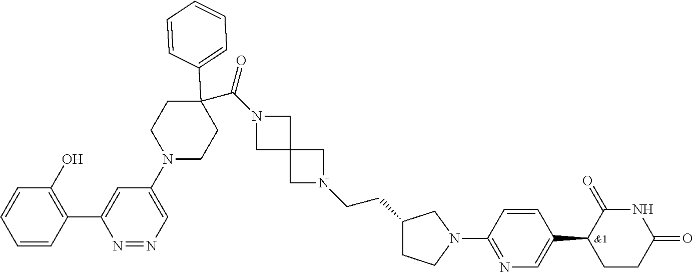 [SMILES: O=C1CC[C@@H](c2ccc(N3CC[C@H](CCN4CC5(C4)CN(C(=O)C4(c6ccccc6)CCN(c6cnnc(-c7ccccc7O)c6)CC4)C5)C3)nc2)C(=O)N1]

This compound was synthesized according to Example 122 using 2-[(3R)-1-{5-[(3RS)-2,6-dioxopiperidin-3-yl]pyridin-2-yl}pyrrolidin-3-yl]acetaldehyde.

### Example 182

### N-{5-[(1-{4-[(3R)-2,6-dioxopiperidin-3-yl]phenyl}piperidin-4-yl)methyl]-5-azaspiro[3.5]nonan-8-yl}-1-[6-(2-hydroxyphenyl)pyridazin-4-yl]-4-(pyridin-2-yl)piperidine-4-carboxamide

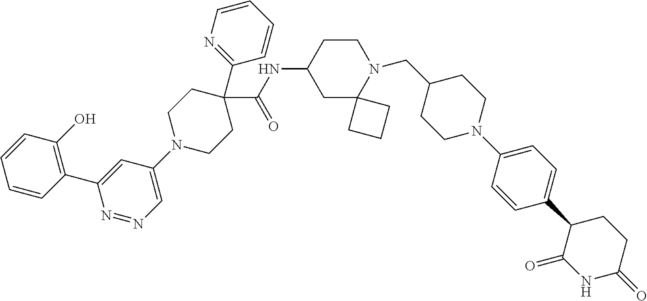 [SMILES: O=C1CC[C@H](c2ccc(N3CCC(CN4CCC(NC(=O)C5(c6ccccn6)CCN(c6cnnc(-c7ccccc7O)c6)CC5)CC45CCC5)CC3)cc2)C(=O)N1]

LCMS: C₄₆H₅₄N₈O₄ requires: 782.4. found: m/z=783.4 [M+H]⁺

### Example 183

### N-{1-[(1-{4-[(3R)-2,6-dioxopiperidin-3-yl]phenyl}piperidin-4-yl)methyl]-2,2-dimethylpiperidin-4-yl}-1-[6-(2-hydroxyphenyl)pyridazin-4-yl]-4-phenylpiperidine-4-carboxamide

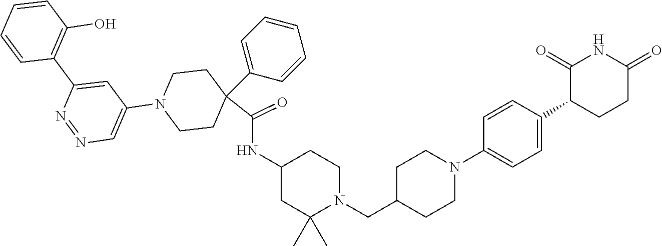 [SMILES: CC1(C)CC(NC(=O)C2(c3ccccc3)CCN(c3cnnc(-c4ccccc4O)c3)CC2)CCN1CC1CCN(c2ccc([C@H]3CCC(=O)NC3=O)cc2)CC1]

The following intermediate was synthesized according to step 1 of Example 106 using 1-[6-(2-hydroxyphenyl)pyridazin-4-yl]-4-phenylpiperidine-4-carboxylic acid and tert-butyl 4-amino-2,2-dimethylpiperidine-1-carboxylate. LCMS: C₂₉H₃₅N₅O₂ requires: 485.3. found: m/z=486.7 [M+H]⁺.

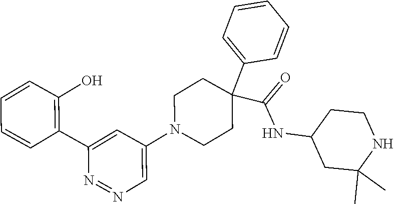 [SMILES: CC1(C)CC(NC(=O)C2(c3ccccc3)CCN(c3cnnc(-c4ccccc4O)c3)CC2)CCN1]

The title compound was synthesized according to step 2 of Example 186. LCMS: C₄₆H₅₅N₇O₄ requires: 769.4. found: m/z=770.4 [M+H]⁺.

### Example 184

### N-{1-[(1-{4-[(3R)-2,6-dioxopiperidin-3-yl]phenyl}piperidin-4-yl)methyl]piperidin-4-yl}-N-ethyl-1-[6-(2-hydroxyphenyl)pyridazin-4-yl]-4-phenylpiperidine-4-carboxamide

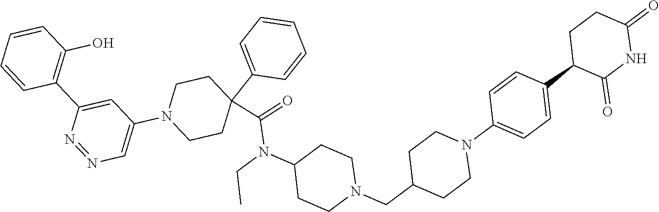 [SMILES: CCN(C(=O)C1(c2ccccc2)CCN(c2cnnc(-c3ccccc3O)c2)CC1)C1CCN(CC2CCN(c3ccc([C@H]4CCC(=O)NC4=O)cc3)CC2)CC1]

Combined N-ethyl-1-[6-(2-hydroxyphenyl)pyridazin-4-yl]-4-phenyl-N-(piperidin-4-yl)piperidine-4-carboxamide; tris(trifluoroacetic acid) (20.00 mg, 0.0242 mmol) and 1-{4-[(3R)-2,6-dioxopiperidin-3-yl]phenyl}piperidine-4-carbaldehyde (7.98 mg, 0.0266 mmol) in DMA (0.20 mL) with N,N-diisopropylethylamine (21.04 μL, 0.02 g, 0.1208 mmol) and then added sodium triacetoxyborohydride (15.36 mg, 0.0725 mmol) and stirred overnight. Added additional portions of DIPEA, aldehyde and STAB and stirred for 4 h. Purified the solution directly by RP-HPLC to yield the title compound (0.0034 g, 18.27%). LCMS: C₄₆H₅₅N₇O₄ requires: 769.4. found: m/z=770.4 [M+H]⁺.

### Example 185

### rac-N-{1-[2-(4-{4-[(3R)-2,6-dioxopiperidin-3-yl]phenyl}piperazin-1-yl)acetyl]piperidin-4-yl}-N-ethyl-1-[6-(2-hydroxyphenyl)pyridazin-4-yl]-4-phenylpiperidine-4-carboxamide

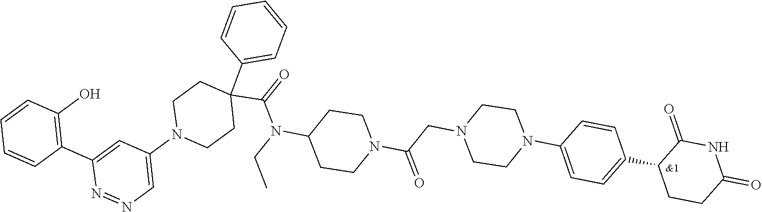 [SMILES: CCN(C(=O)C1(c2ccccc2)CCN(c2cnnc(-c3ccccc3O)c2)CC1)C1CCN(C(=O)CN2CCN(c3ccc([C@H]4CCC(=O)NC4=O)cc3)CC2)CC1]

Combined a solution of N-ethyl-1-[6-(2-hydroxyphenyl)pyridazin-4-yl]-4-phenyl-N-(piperidin-4-yl)piperidine-4-carboxamide; tris(trifluoroacetic acid) (20.00 mg, 0.0242 mmol) in 100 μL DMA, with N,N-diisopropylethylamine (25.32 μL, 0.02 g, 0.1450 mmol) and a solution of rac-(4-{4-[(3R)-2,6-dioxopiperidin-3-yl]phenyl}piperazin-1-yl)acetic acid (8.81 mg, 0.0266 mmol) in 100 μL DMA and then added a solution of [(dimethylamino)({[1,2,3]triazolo[4,5-b]pyridin-3-yloxy})methylidene]dimethylazanium; hexafluoro-lambda5-phosphanuide (10.11 mg, 0.0266 mmol) in 100 μL DMA and stirred at rt overnight. Purified the solution directly by RP-HPLC to yield the title compound (0.0022 g, 11.40%). LCMS: C₄₆H₅₄N₈O₅ requires: 798.4. found: m/z=799.4 [M+H]+¹H NMR (500 MHz, DMSO) δ 14.65 (s, 1H), 10.77 (s, 1H), 8.91 (d, J=2.9 Hz, 1H), 8.11 (d, J=7.9 Hz, 1H), 7.53 (d, J=3.0 Hz, 1H), 7.45-7.26 (m, 7H), 7.05 (d, J=8.4 Hz, 2H), 6.95-6.85 (m, 4H), 4.25 (d, J=12.5 Hz, 1H), 4.13 (d, J=13.1 Hz, 1H), 4.05 (s, 1H), 3.91 (d, J=13.0 Hz, 1H), 3.73 (dd, J=11.1, 4.9 Hz, 1H), 3.59 (s, 1H), 3.47 (s, 1H), 3.41 (s, 1H), 3.23 (d, J=13.1 Hz, 1H), 3.13 (d, J=7.4 Hz, 2H), 3.07 (s, 3H), 3.00 (d, J=13.2 Hz, 1H), 2.41 (s, 2H), 2.16-2.06 (m, 3H), 2.04-1.99 (m, 1H), 1.50 (s, 1H), 1.35 (s, 1H), 1.02 (t, J=6.8 Hz, 3H), 0.96 (s, 1H), 0.82 (s, 1H).

### Example 186

### (3R)-3-(4-{4-[2-(6-{1-[6-(2-hydroxyphenyl)pyridazin-4-yl]-4-(5-methyl-1,2-oxazol-3-yl)piperidine-4-carbonyl}-2,6-diazaspiro[3.3]heptan-2-yl)ethyl]piperidin-1-yl}phenyl)piperidine-2,6-dione

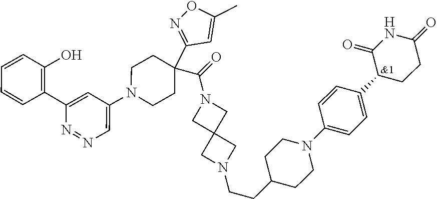 [SMILES: Cc1cc(C2(C(=O)N3CC4(CN(CCC5CCN(c6ccc([C@H]7CCC(=O)NC7=O)cc6)CC5)C4)C3)CCN(c3cnnc(-c4ccccc4O)c3)CC2)no1]

### Step 1: Synthesis of 2-[5-(4-{2,6-diazaspiro[3.3]heptane-2-carbonyl}-4-(5-methyl-1,2-oxazol-3-yl)piperidin-1-yl)pyridazin-3-yl]phenol

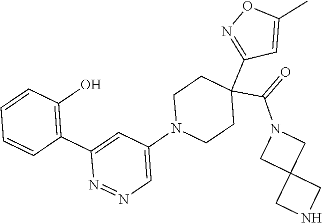 [SMILES: Cc1cc(C2(C(=O)N3CC4(CNC4)C3)CCN(c3cnnc(-c4ccccc4O)c3)CC2)no1]

A solution of 1-[6-(2-hydroxyphenyl)pyridazin-4-yl]-4-(5-methyl-1,2-oxazol-3-yl)piperidine-4-carboxylic acid (Intermediate 1) (14.00 mg, 0.0368 mmol) in 0.2 mL DMA was added to tert-butyl 2,6-diazaspiro[3.3]heptane-2-carboxylate (7.30 mg, 0.0368 mmol) with N,N-diisopropylethylamine (19.23 μL, 0.01 g, 0.1104 mmol) and then [(dimethylamino)({[1,2,3]triazolo[4,5-b]pyridin-3-yloxy})methylidene]dimethylazanium; hexafluoro-lambda5-phosphanuide (16.79 mg, 0.0442 mmol) was added as a solution in 0.2 mL DMA. Stirred at rt. Added an additional portion of amine, DIPEA and HATU. Diluted with DCM (3 mL) and washed with 1M citric acid (2×4 mL) and sodium bicarbonate (1×4 ml), then passed through a phase separator. Added TFA (2 mL), then after 45 min, concentrated to dryness. Dissolved in 1:1 ACN/water (2 mL) and lyophilized. Used in the next step as is. LCMS: C₂₉H₃₉N₅O₃ requires: 460.2. found: m/z=461.6 [M+H]⁺,

### Step 2: Synthesis of the Title Compound

Combined a solution of 2-[5-(4-{2,6-diazaspiro[3.3]heptane-2-carbonyl}-4-(5-methyl-1,2-oxazol-3-yl)piperidin-1-yl)pyridazin-3-yl]phenol (6.00 mg, 0.0130 mmol) in 0.1 mL DMA to a solution of 2-(1-{4-[(3R)-2,6-dioxopiperidin-3-yl]phenyl}piperidin-4-yl)acetaldehyde (4.10 mg, 0.0130 mmol) in 0.1 mL DMA with N,N-diisopropylethylamine (11.35 μL, 0.01 g, 0.0651 mmol) and then added a solution of sodium triacetoxyborohydride (8.28 mg, 0.0391 mmol) in 0.1 mL DMA and stirred overnight. Submitted to HTP to give (3R)-3-(4-{4-[2-(6-{1-[6-(2-hydroxyphenyl)pyridazin-4-yl]-4-(5-methyl-1,2-oxazol-3-yl)piperidine-4-carbonyl}-2,6-diazaspiro[3.3]heptan-2-yl)ethyl]piperidin-1-yl}phenyl)piperidine-2,6-dione (0.0049 g, 49.16%).

LCMS: C₄₃H₅₀N₈O₅ requires: 758.4. found: m/z=759.4 [M+H]⁺.

### Example 187

### N-{1-[2-(1-{4-[(3R)-2,6-dioxopiperidin-3-yl]phenyl}piperidin-4-yl)ethyl]piperidin-4-yl}-n-ethyl-1-[6-(2-hydroxyphenyl)pyridazin-4-yl]-4-phenylpiperidine-4-carboxamide

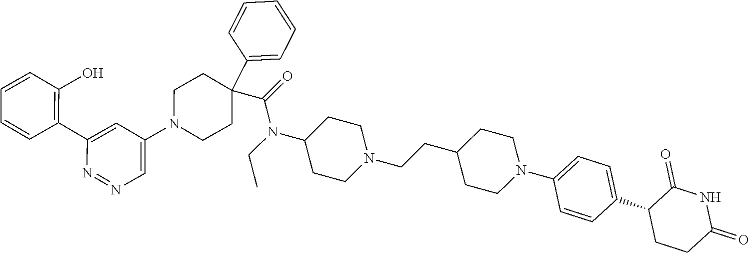 [SMILES: CCN(C(=O)C1(c2ccccc2)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of tert-butyl 4-(N-ethyl-1-{6-[2-(methoxymethoxy)phenyl]pyridazin-4-yl}-4-phenylpiperidine-4-amido)piperidine-1-carboxylate

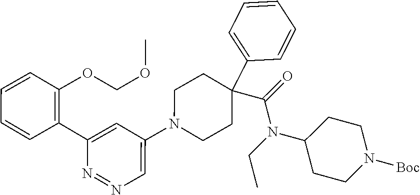 [SMILES: CCN(C(=O)C1(c2ccccc2)CCN(c2cnnc(-c3ccccc3OCOC)c2)CC1)C1CCN(C(=O)OC(C)(C)C)CC1]

1-{6-[2-(methoxymethoxy)phenyl]pyridazin-4-yl}-4-phenylpiperidine-4-carboxylic acid (316.00 mg, 0.7533 mmol) and tert-butyl 4-(ethylamino)piperidine-1-carboxylate (1026.91 μL, 1032.05 mg, 4.5199 mmol) were dissolved in DMA (2.00 mL) and N,N-diisopropylethylamine (656.09 μL, 0.49 g, 3.7666 mmol) and then [(dimethylamino)({[1,2,3]triazolo[4,5-b]pyridin-3-yloxy})methylidene]dimethylazanium; hexafluoro-lambda5-phosphanuide (859.31 mg, 2.2599 mmol) was added. Stirred at 50 C overnight. Added more HATU (3 eq) and stirred over the weekend at 50 C. Partitioned between DCM and sodium bicarbonate. Purified by ISCO RP-HPLC. tert-butyl 4-(N-ethyl-1-{6-[2-(methoxymethoxy)phenyl]pyridazin-4-yl}-4-phenylpiperidine-4-amido)piperidine-1-carboxylate (0.158 g, 33.30%). LCMS: C₃₆H₄₇N₅O₅ requires: 629.8. found: m/z=630.4 [M+H]⁺.

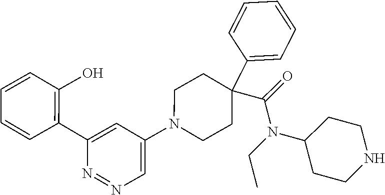 [SMILES: CCN(C(=O)C1(c2ccccc2)CCN(c2cnnc(-c3ccccc3O)c2)CC1)C1CCNCC1]

### Step 2: Synthesis of N-ethyl-1-[6-(2-hydroxyphenyl)pyridazin-4-yl]-4-phenyl-N-(piperidin-4-yl)piperidine-4-carboxamide

Dissolved tert-butyl 4-(N-ethyl-1-{6-[2-(methoxymethoxy)phenyl]pyridazin-4-yl}-4-phenylpiperidine-4-amido)piperidine-1-carboxylate (140.00 mg, 0.2223 mmol) in 1:1 TFA/DCM (4 mL). The reaction was allowed to sit for 30 min, then the solvent was removed under vacuum. Redissolved in ACN and reconcentrated under vacuum two times. Used directly in the next step without further purification. LCMS: C₂₉H₃₅N₅O₂ requires: 485.3. found: m/z=486.4 [M+H]⁺.

### Step 3: Synthesis of the Title Compound

Combined N-ethyl-1-[6-(2-hydroxyphenyl)pyridazin-4-yl]-4-phenyl-N-(piperidin-4-yl)piperidine-4-carboxamide; trifluoroacetic acid (60.80 mg, 0.1014 mmol) and 2-(1-{4-[(3R)-2,6-dioxopiperidin-3-yl]phenyl}piperidin-4-yl)acetaldehyde (35.06 mg, 0.1115 mmol) in DMA (0.60 mL) with N,N-diisopropylethylamine (88.31 μL, 0.07 g, 0.5070 mmol) and then added sodium triacetoxyborohydride (64.47 mg, 0.3042 mmol) and stirred for 1 h. The reaction was purified directly by MP-HPLC, then chiral SFC and then RP-HPLC to yield the title compound (0.0226 g, 28.43%). LCMS: C₄₇H₅₇N₇O₄ requires: 783.4. found: m/z=784.4 [M+H]^{+ 1}H NMR (500 MHz, DMSO) δ 14.65 (s, 1H), 10.76 (s, 1H), 8.91 (d, J=2.9 Hz, 1H), 8.11 (dd, J=8.5, 1.7 Hz, 1H), 7.52 (d, J=2.9 Hz, 1H), 7.40 (t, J=7.7 Hz, 2H), 7.36-7.29 (m, 3H), 7.27 (t, J=7.3 Hz, 1H), 7.04-6.99 (m, 2H), 6.95-6.89 (m, 2H), 6.89-6.83 (m, 2H), 4.12 (d, J=13.1 Hz, 2H), 3.70 (dd, J=10.9, 4.9 Hz, 1H), 3.60 (d, J=12.1 Hz, 2H), 3.40 (t, J=12.1 Hz, 2H), 3.17 (d, J=7.1 Hz, 2H), 2.67 (d, J=11.1 Hz, 2H), 2.64-2.52 (m, 4H), 2.44 (dd, J=17.3, 4.6 Hz, 2H), 2.34 (s, 1H), 2.13 (dt, J=19.9, 5.8 Hz, 3H), 2.04 (s, 1H), 1.99 (dd, J=13.3, 5.0 Hz, 2H), 1.67 (d, J=12.5 Hz, 2H), 1.49 (d, J=11.9 Hz, 1H), 1.35 (d, J=11.8 Hz, 2H), 1.33-1.25 (m, 3H), 1.20 (dd, J=22.3, 11.1 Hz, 2H), 1.04 (t, J=6.8 Hz, 3H), 0.85 (s, 2H).

### Example 188

### N-[(8RS&)-5-[(1-{4-[(3RS)-2,6-dioxopiperidin-3-yl]-3,5-difluorophenyl}piperidin-4-yl)methyl]-5-azaspiro[3.5]nonan-8-yl]-1-[6-(2-hydroxyphenyl)pyridazin-4-yl]-4-phenylpiperidine-4-carboxamide

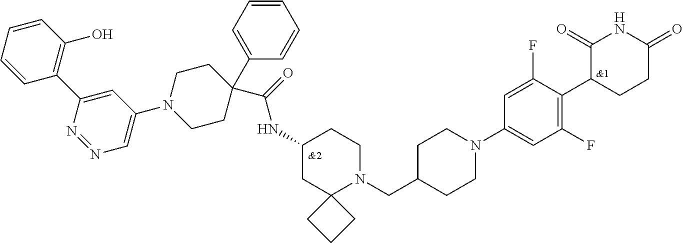 [SMILES: O=C1CCC(c2c(F)cc(N3CCC(CN4CC[C@@H](NC(=O)C5(c6ccccc6)CCN(c6cnnc(-c7ccccc7O)c6)CC5)CC45CCC5)CC3)cc2F)C(=O)N1]

Combined a solution of N-{5-azaspiro[3.5]nonan-8-yl}-1-[6-(2-hydroxyphenyl)pyridazin-4-yl]-4-phenylpiperidine-4-carboxamide; trifluoroacetic acid (20.00 mg, 0.0327 mmol) in 0.1 mL DMA to a solution of rac-1-{4-[(3R)-2,6-dioxopiperidin-3-yl]-3,5-difluorophenyl}piperidine-4-carbaldehyde (12.10 mg, 0.0360 mmol) in 0.1 mL DMA with N,N-diisopropylethylamine (34.17 μL, 0.03 g, 0.1962 mmol) and then added a solution of sodium triacetoxyborohydride (20.79 mg, 0.0981 mmol) in 0.1 mL DMA and stirred for 2 h. Added more aldehyde (10 mg) and STAB (10 mg) and stirred overnight. Added more aldehyde (5 mg) and STAB (10 mg). Purified the solution directly by RP-HPLC. The resulting solid was dissolved in DCM and washed with sat bicarbonate three times, then the organic layer was passed through a phase separator. The material was then freeze dried from 1:1 ACN/water to yield the title compound (0.0097 g, 35.65%). LCMS: C₄₇H₅₃F₂N₇O₄ requires: 817.4, found: m/z=818.3 [M+H]^{+ 1}H NMR (500 MHz, DMSO) δ 14.65 (s, 1H), 10.87 (s, 1H), 8.99 (d, J=2.9 Hz, 1H), 8.16-8.10 (m, 1H), 7.58 (d, J=3.0 Hz, 1H), 7.52 (d, J=8.0 Hz, 1H), 7.43-7.30 (m, 5H), 7.25 (t, J=7.2 Hz, 1H), 6.96-6.89 (m, 2H), 6.60 (d, J=13.2 Hz, 2H), 4.14 (d, J=13.2 Hz, 2H), 4.04 (dd, J=12.6, 5.2 Hz, 1H), 3.75 (s, 3H), 3.22 (t, J=12.3 Hz, 2H), 2.84-2.72 (m, 2H), 2.69 (d, J=12.2 Hz, 2H), 2.62 (s, 2H), 2.34 (d, J=12.6 Hz, 1H), 2.16-1.98 (m, 3H), 1.91 (d, J=13.7 Hz, 3H), 1.81 (s, 2H), 1.76-1.66 (m, 3H), 1.63 (s, 1H), 1.52 (s, 3H), 1.32 (t, J=12.3 Hz, 2H), 1.10 (d, J=12.7 Hz, 2H).

### Example 189

### (3R)-3-(6-{4-[(2-{1-[6-(2-hydroxyphenyl)pyridazin-4-yl]-4-phenylpiperidine-4-carbonyl}-2,7-diazaspiro[3.5]nonan-7-yl)methyl]piperidin-1-yl}pyridin-3-yl)piperidine-2,6-dione

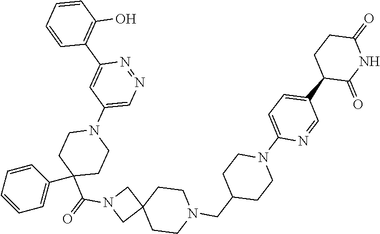 [SMILES: O=C1CC[C@H](c2ccc(N3CCC(CN4CCC5(CC4)CN(C(=O)C4(c6ccccc6)CCN(c6cnnc(-c7ccccc7O)c6)CC4)C5)CC3)nc2)C(=O)N1]

LCMS: C₄₅H₅₂N₈O₄ requires: 768.4. found: m/z=769.3 [M+H]⁺

### Example 190

### N-[(8R)-5-[(1-{4-[(3R)-2,6-dioxopiperidin-3-yl]phenyl}piperidin-4-yl)methyl]-5-azaspiro[3.5]nonan-8-yl]-1-[6-(2-hydroxyphenyl)pyridazin-4-yl]-4-phenylpiperidine-4-carboxamide

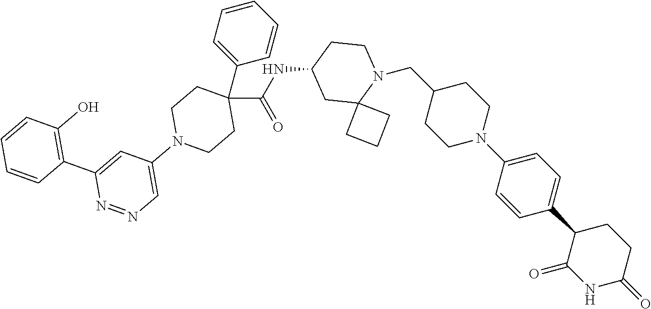 [SMILES: O=C1CC[C@H](c2ccc(N3CCC(CN4CC[C@@H](NC(=O)C5(c6ccccc6)CCN(c6cnnc(-c7ccccc7O)c6)CC5)CC45CCC5)CC3)cc2)C(=O)N1]

LCMS: C₄₇H₅₅N₇O₄ requires: 781.4. found: m/z=782.4 [M+H]⁺

### Examples 191A and 191B

### N-[(8S)-5-[(1-{4-[(3R)-2,6-dioxopiperidin-3-yl]phenyl}piperidin-4-yl)methyl]-5-azaspiro[3.5]nonan-8-yl]-1-[6-(2-hydroxyphenyl)pyridazin-4-yl]-4-phenoxypiperidine-4-carboxamide and N-[(8R)-5-[(1-{4-[(3R)-2,6-dioxopiperidin-3-yl]phenyl}piperidin-4-yl)methyl]-5-azaspiro[3.5]nonan-8-yl]-1-[6-(2-hydroxyphenyl)pyridazin-4-yl]-4-phenoxypiperidine-4-carboxamide

### Step 1: Synthesis of N-{5-azaspiro[3.5]nonan-8-yl}-1-[6-(2-hydroxyphenyl)pyridazin-4-yl]-4-phenoxypiperidine-4-carboxamide

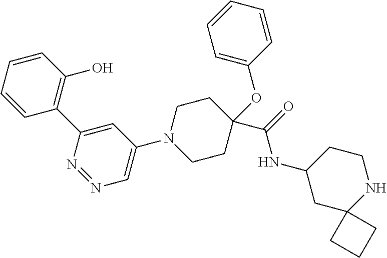 [SMILES: O=C(NC1CCNC2(CCC2)C1)C1(Oc2ccccc2)CCN(c2cnnc(-c3ccccc3O)c2)CC1]

This intermediate was synthesized according to Step 1 in Example 136 using tert-butyl 8-amino-5-azaspiro[3.5]nonane-5-carboxylate. LCMS: C₃₀H₃₅N₅O₃ requires: 513.3. found: m/z=514.7 [M+H]⁺.

### Step 2: Synthesis of the Title Compounds

Combined N-{5-azaspiro[3.5]nonan-8-yl}-1-[6-(2-hydroxyphenyl)pyridazin-4-yl]-4-phenoxypiperidine-4-carboxamide (134.00 mg, 0.2609 mmol) and 1-{4-[(3R)-2,6-dioxopiperidin-3-yl]phenyl}piperidine-4-carbaldehyde (101.87 mg, 0.3391 mmol) in DMA (3.00 mL) with N,N-diisopropylethylamine (227.21 μL, 0.17 g, 1.3044 mmol) and then added sodium triacetoxyborohydride (165.87 mg, 0.7826 mmol) and stirred for 2 h. Purified the solution directly by RP-HPLC to yield the mixture of isomers. Chiral SFC was performed to obtain 2 peaks with opposite stereochemistry at the linker. Stereochemistry assigned arbitrarily.

SFC Prep Method: Sample Dissolution—100 mg dissolved into 10 ml of 95% Acetonitrile, 5% Water: Column—Regis, (R,R) Whelk-01, 15 cm×21.1 mm Cat #780209, Isocratic Conditions, Flow Rate—100 ml/min: Solvent A—40%, CO₂ Solvent B—60% Mixture of (40% Ethanol: 60% Acetonitrile)+0.1% Ammonia Hydroxide by volume: BPR—100 Bar: Oven Temperature—40 C: Injection Volume—1000 μL: Runtime=6 minutes: UV—254 nm: The later 2 eluting major peaks were collected.

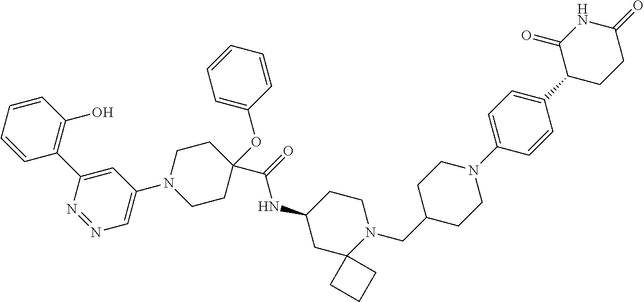 [SMILES: O=C1CC[C@H](c2ccc(N3CCC(CN4CC[C@H](NC(=O)C5(Oc6ccccc6)CCN(c6cnnc(-c7ccccc7O)c6)CC5)CC45CCC5)CC3)cc2)C(=O)N1]

Peak 1: LCMS: C₄₇H₅₅N₇O₅ requires: 797.4. found: m/z=798.4 [M+H]⁺

¹H NMR (500 MHz, DMSO) δ 14.58 (s, 1H), 10.76 (s, 1H), 8.96 (d, J=2.9 Hz, 1H), 8.14 (d, J=8.2 Hz, 1H), 8.12-8.07 (m, 1H), 7.56 (d, J=3.0 Hz, 1H), 7.33 (tt, J=7.1, 1.9 Hz, 3H), 7.02 (s, 1H), 7.05-6.98 (m, 2H), 6.98-6.84 (m, 6H), 4.06 (s, 2H), 3.82 (s, 1H), 3.70 (dd, J=10.9, 4.9 Hz, 1H), 3.64 (s, 2H), 3.39-3.33 (m, 1H), 3.28 (d, J=8.1 Hz, 1H), 2.66 (s, 1H), 2.64-2.52 (m, 2H), 2.49-2.41 (m, 1H), 2.37 (t, J=12.7 Hz, 1H), 2.10 (s, 1H), 2.04-1.96 (m, 1H), 1.89 (dd, J=19.7, 8.0 Hz, 1H), 1.81 (s, 2H), 1.71 (dt, J=21.3, 10.8 Hz, 1H), 1.65 (s, 1H), 1.56-1.47 (m, 1H), 1.45-1.33 (m, 2H), 1.31 (d, J=12.0 Hz, 1H), 1.16 (s, 2H).

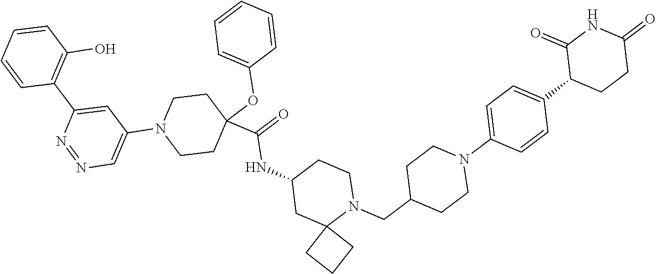 [SMILES: O=C1CC[C@H](c2ccc(N3CCC(CN4CC[C@@H](NC(=O)C5(Oc6ccccc6)CCN(c6cnnc(-c7ccccc7O)c6)CC5)CC45CCC5)CC3)cc2)C(=O)N1]

Peak 2: LCMS: C₄₇H₅₅N₇O₅ requires: 797.4. found: m/z=798.4 [M+H]⁺

¹H NMR (500 MHz, DMSO) δ 14.58 (s, 1H), 10.76 (s, 1H), 8.96 (d, J=2.9 Hz, 1H), 8.14 (d, J=8.3 Hz, 1H), 8.12-8.07 (m, 1H), 7.56 (d, J=3.0 Hz, 1H), 7.32 (tt, J=7.4, 1.4 Hz, 3H), 7.05-6.98 (m, 3H), 6.98-6.84 (m, 5H), 4.06 (d, J=4.2 Hz, 2H), 3.82 (d, J=7.9 Hz, 1H), 3.70 (dd, J=10.9, 5.0 Hz, 1H), 3.64 (t, J=11.2 Hz, 2H), 3.39-3.33 (m, 1H), 2.62 (dt, J=24.3, 12.3 Hz, 5H), 2.49-2.37 (m, 2H), 2.11 (s, 3H), 2.07-1.96 (m, 2H), 1.94-1.82 (m, 3H), 1.81 (s, 1H), 1.78-1.70 (m, 1H), 1.66 (dd, J=17.1, 8.8 Hz, 2H), 1.56-1.47 (m, 2H), 1.36 (td, J=28.1, 14.5 Hz, 3H), 1.15 (d, J=13.9 Hz, 2H).

### Example 192

### N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N-methyl-4-phenylpiperidine-4-carboxamide

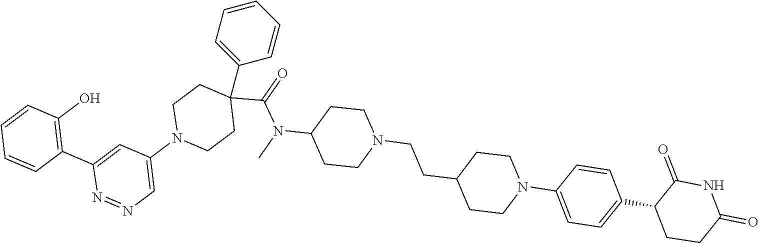 [SMILES: CN(C(=O)C1(c2ccccc2)CCN(c2cnnc(-c3ccccc3O)c2)CC1)C1CCN(CCC2CCN(c3ccc([C@H]4CCC(=O)NC4=O)cc3)CC2)CC1]

This compound was synthesized according to step 3 of Example 187 using 1-[6-(2-hydroxyphenyl)pyridazin-4-yl]-N-methyl-4-phenyl-N-(piperidin-4-yl)piperidine-4-carboxamide; tris(trifluoroacetic acid). LCMS: C₄₆H₅₅N₇O₄ requires: 769.4. found: m/z=770.3 [M+H]^{+ 1}H NMR (500 MHz, DMSO) δ 14.63 (s, 1H), 10.77 (s, 1H), 8.93 (s, 1H), 8.10 (d, J=8.0 Hz, 1H), 7.52 (d, J=2.8 Hz, 1H), 7.41 (t, J=7.6 Hz, 2H), 7.37-7.26 (m, 4H), 7.03 (d, J=8.1 Hz, 3H), 6.96-6.87 (m, 4H), 6.87 (s, 1H), 3.72 (dd, J=11.0, 4.9 Hz, 1H), 3.65 (s, 3H), 3.08 (s, 2H), 2.67 (s, 2H), 2.62 (dd, J=11.1, 5.5 Hz, 1H), 2.49-2.42 (m, 1H), 2.40 (s, 4H), 2.15-2.06 (m, 1H), 2.04-1.97 (m, 1H), 1.72 (s, 2H), 1.52 (s, 3H), 1.24 (s, 5H), 0.86 (t, J=6.7 Hz, 1H).

### Example 193

### rac-(3R)-3-(2,6-difluoro-4-{4-[(2-{1-[6-(2-hydroxyphenyl)pyridazin-4-yl]-4-phenylpiperidine-4-carbonyl}-2,7-diazaspiro[3.5]nonan-7-yl)methyl]piperidin-1-yl}phenyl)piperidine-2,6-dione

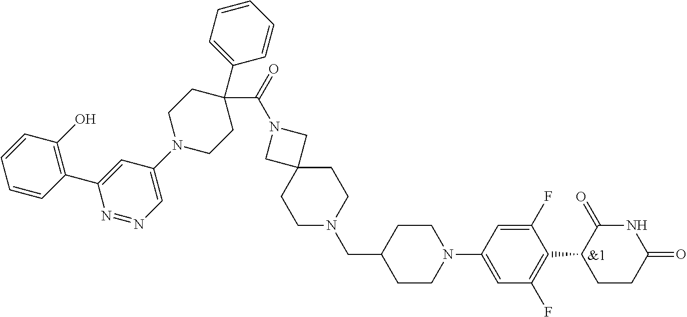 [SMILES: O=C1CC[C@H](c2c(F)cc(N3CCC(CN4CCC5(CC4)CN(C(=O)C4(c6ccccc6)CCN(c6cnnc(-c7ccccc7O)c6)CC4)C5)CC3)cc2F)C(=O)N1]

LCMS: C₄₆H₅₁F₂N₇O₄ requires: 803.4. found: m/z=804.3 [M+H]⁺.

### Example 194

### (3R)-3-(4-{4-[(2-{1-[6-(2-hydroxyphenyl)pyridazin-4-yl]-4-phenylpiperidine-4-carbonyl}-2,7-diazaspiro[3.5]nonan-7-yl)methyl]piperidin-1-yl}phenyl)piperidine-2,6-dione

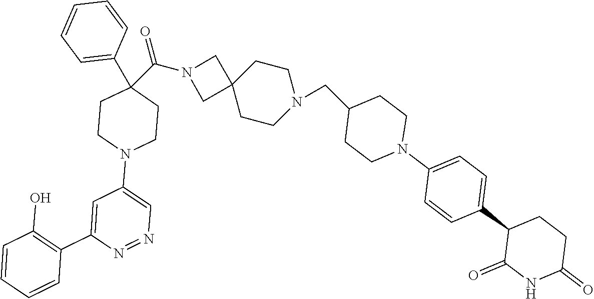 [SMILES: O=C1CC[C@H](c2ccc(N3CCC(CN4CCC5(CC4)CN(C(=O)C4(c6ccccc6)CCN(c6cnnc(-c7ccccc7O)c6)CC4)C5)CC3)cc2)C(=O)N1]

LCMS: C₄₆H₅₃N₇O₄ requires: 767.4. found: m/z=768.4 [M+H]⁺

### Example 195

### rac-N-(1-(2-(1-(4-(2,6-dioxopiperidin-3-yl)phenyl)piperidin-4-yl)ethyl)piperidin-4-yl)-1-(6-(2-hydroxyphenyl)pyridazin-4-yl)-4-methoxy-n-methylpiperidine-4-carboxamide

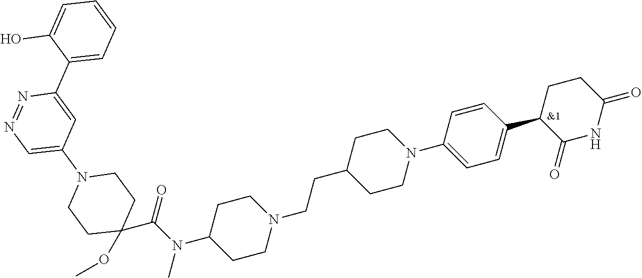 [SMILES: COC1(C(=O)N(C)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of Title Compound

1-(6-(2-hydroxyphenyl)pyridazin-4-yl)-4-methoxy-N-methyl-N-(piperidin-4-yl)piperidine-4-carboxamide (9.36 mg, 0.022 mmol) and rac-2-(1-(4-(2,6-dioxopiperidin-3-yl)phenyl)piperidin-4-yl)acetaldehyde (Intermediate 28) (6.91 mg, 0.022 mmol) were dissolved in 0.5 mL DMA. N,N-diisopropylethylamine (14.3 mg, 0.110 mmol) was added followed by addition of sodium triacetoxyborohydride (23.3 mg, 0.110 mmol) in one portion and the resulting solution was stirred at room temperature for 1 h. The reaction was quenched with 0.1 mL H₂O and directly injected onto RP-FC and purified with a gradient of 0-80% MeCN in H₂O to yield the title compound (7.0 mg, 43.9%). LCMS: C₄₁H₅₃N₇O₅ requires 723.4. found: m/z=724.4 [M+H]⁺.

### Example 196

### (r)-n-(1-(2-(1-(4-(2,6-dioxopiperidin-3-yl)phenyl)piperidin-4-yl)ethyl)piperidin-4-yl)-1-(6-(2-hydroxyphenyl)pyridazin-4-yl)-4-methoxy-n-methylpiperidine-4-carboxamide

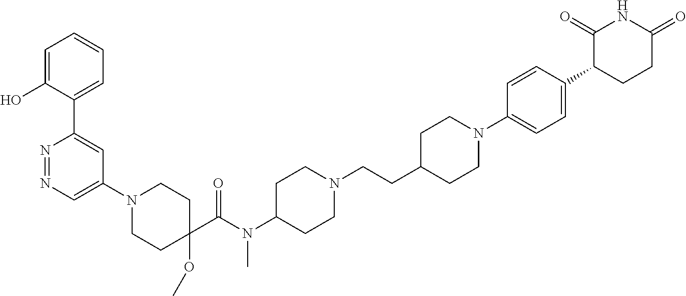 [SMILES: COC1(C(=O)N(C)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of Title Compound

1-(6-(2-hydroxyphenyl)pyridazin-4-yl)-4-methoxy-N-methyl-N-(piperidin-4-yl)piperidine-4-carboxamide (19.15 mg, 0.045 mmol) and rac-2-(1-(4-(2,6-dioxopiperidin-3-yl)phenyl)piperidin-4-yl)acetaldehyde (Intermediate 28) (16.98 mg, 0.054 mmol) were dissolved in 1.0 mL DMA. N,N-diisopropylethylamine (29.1 mg, 0.225 mmol) was added followed by addition of sodium triacetoxyborohydride (28.6 mg, 0.135 mmol) in one portion and the resulting solution was stirred at room temperature for 1 h. The reaction was quenched with 0.1 mL H₂O and directly injected onto RP-FC and purified with a gradient of 0-80% MeCN in H₂O. The isolated material was then subjected to chiral SFC purification (60% MeCN in EtOH, 0.1% NH₄0H) to afford R—N-(1-(2-(1-(4-(2,6-dioxopiperidin-3-yl)phenyl)piperidin-4-yl)ethyl)piperidin-4-yl)-1-(6-(2-hydroxyphenyl)pyridazin-4-yl)-4-methoxy-N-methylpiperidine-4-carboxamide (8.0 mg, 24.6%).

LCMS: C₄₁H₅₃N₇O₅ requires 723.4. found: m/z=724.4 [M+H]⁺.

### Biological Example

### SMARCA2 Cellular Degradation Assay

### Western Blot Analysis for SMARCA2 Degradation

Frozen cell pellets were thawed on ice and resuspended in RIPA lysis buffer (Millipore, Cat. No. 20-188) supplemented with 0.2% SDS (Fisher Scientific, Cat. No. 24730020), 200 mM β-glycerophosphate (Alfa Aesar, Cat. No. J62121), 500 mM NaF (Rigaku, Cat. No. 1008046), protease inhibitor (1 tablet per 10 mL, Sigma, Cat. No. 11836170001), 250 U/μL benzonase (Millipore, Cat. No. 71206), 200 mM sodium orthovanadate (FivePhoton, Cat. No. ActVO-4), and 100 mM PMSF (Sigma, Cat. No. 10837091001). 50 μL lysis buffer was used to lyse each pellet containing 5×10E5-1×10E6 live cells extracted from tumors. Lysates were centrifuged for 10 mins at 20,817×g and lysate supernatants were saved for further analysis. Protein levels were determined by the BCA Assay performed according to manufacturer's protocol (EMD Millipore, Cat. No. 71285-3). Samples were evaluated by Western blot analysis on the Jess Platform (ProteinSimple). Lysates were diluted with complete lysis buffer (as described above) and 5 μg of total proteins were loaded onto each capillary. Samples were prepared using the ProteinSimple EZ Standard Pack 1 (Cat. No. PS-ST01EZ-8; containing tubes with powdered (5×) Fluorescent Master Mix, DTT, and biotinylated ladder) and run using the ProteinSimple 25-Capillary Cartridge kit (Cat. No. SM-W004; containing 25-capillary cartridge Wash reagent, and 10× Sample Buffer reagent), the ProteinSimple Anti-Rabbit HRP Detection Module for Jess (Cat. No. DM-001; containing Antibody Diluent, anti-rabbit secondary antibody, Luminol-S, Peroxide, and Streptavidin-HRP), and the ProteinSimple Anti-Mouse NIR Detection Module for Jess (Cat. No. DM-009; containing Antibody Diluent, anti-mouse NIR secondary antibody, and Streptavidin-NIR) as the following describes. The DTT was reconstituted with 40 μL water to make a 400 mM solution, 20 μL of the DTT solution plus 20 μL of 10× Sample Buffer were added to reconstitute the 5× Fluorescent Master Mix, and 20 μL of H₂O was used to reconstitute the biotinylated ladder. For each sample, lysate (4 μL) was combined with 5× Fluorescent Master Mix (1 μL) and heated at 95° C. for 5 minutes. Samples were cooled and stored on ice. Primary antibody solution was made using anti-SMARCA2 antibody (Abcam, Cat. No. 240648, 1:10) and anti-beta-Actin antibody (Cell Signaling Technology, Cat. No. 3700S, 1:25) in antibody diluent. The 25-capillary cartridges were prepared per standard ProteinSimple protocol with each sample in a column. For the biotinylated ladder in column 1, the following were added: 5 L of ladder sample in Row A, 10 μL of antibody diluent in Rows B and C, 10 μL of Streptavidin-NIR in Row D, and 15 μL of a 50:50 Luminol-s/Peroxide solution in Row E. For each test sample (columns 2-25), the following were added per column: 5 μL of sample in Row A, 10 μL of antibody diluent in Row B, 10 μL of primary antibody solution in Row C, 10 μL of secondary antibody in Row D, and 15 μL of a 50:50 Luminol-S/Peroxide solution in Row E. 500 μL wash buffer was added to the wash buffer wells in the cartridge and cartridge was spun at 1000×g for 5 minutes prior to loading into the Jess. Samples were run using the Chemiluminescence and Fluorescence immunoassays in the ProteinSimple Compass for SimpleWestern program (version 5.0.0). After running, SMARCA2, actin, and background curves were identified in the Compass software; note that in the Compass software, the electropherograms are also visualized as bands for comparison with standard Western blot outputs. The electropherogram area reading for each sample curve was normalized to that of the corresponding actin curve per column following background (capillary background) subtraction using the following equation:

$$Sample Normalized Value = [ \frac{SMARCA 2 Area Value}{β - Actin Area Value} ]$$

Following normalization, the percentage of SMARCA2 remaining per sample relative to the vehicle control was calculated as follows:

$$\% remaining = [ \frac{Example \# / Treated Sample Normalized Value}{Mean ( Vehicle Sample Normalized Value} ] \times 100$$

Table 1 demonstrates the biological activities of certain representative compounds of Formula (I).

TABLE 1
Biological Data

| | | | | | Fold |
|---|---|---|---|---|---|
| Example | Dmax (%) | | DC₅₀ (nM) | | Selectivity |
| No. | SMARCA2 | SMARCA4 | SMARCA2 | SMARCA4 | SMARCA4/2 |
| 1 | 35.16 | 13.03 | 10000 | 10000 | 1 |
| 2 | 53.49 | 22.09 | 10000 | 10000 | 1 |
| 3 | 54.18 | 17.49 | 10000 | 10000 | 1 |
| 4 | 16.43 | 9.4 | 10000 | 10000 | 1 |
| 5 | 59.82 | 22.61 | 462.56 | 10000 | 21.62 |
| 6 | 63.57 | 31.5 | 120.68 | 10000 | 82.86 |
| 7 | 53.09 | 31.96 | 10000 | 10000 | 1 |
| 8 | 75.12 | 44.56 | 41.38 | 10000 | 241.66 |
| 9 | 75.16 | 44.71 | 19.95 | 6715.64 | 336.59 |
| 10 | 59.34 | 25.62 | 80.12 | 10000 | 124.82 |
| 11 | 69.1 | 38.88 | 21.16 | 10000 | 472.68 |
| 12 | 61.69 | 36.9 | 143.54 | 10000 | 69.67 |
| 13 | 40.34 | 12.34 | 10000 | 10000 | 1 |
| 14 | 22.16 | 15.19 | 10000 | 10000 | 1 |
| 15 | 79.94 | 49.29 | 14.77 | 5026.66 | 340.31 |
| 16 | 82.64 | 56.06 | 7.48 | 5022.55 | 671.32 |
| 17 | 78.03 | 54.2 | 13.9 | 4367.69 | 314.31 |
| 18 | 44.07 | 27.73 | 10000 | 10000 | 1 |
| 19 | 77.94 | 57.03 | 24.38 | 156.46 | 6.42 |
| 20 | 67.45 | 48.28 | 24.59 | 10000 | 406.6 |
| 21 | 32.02 | 19.29 | 10000 | 10000 | 1 |
| 22 | 30.48 | 20.58 | 10000 | 10000 | 1 |
| 23 | 55.89 | 39.06 | 78.47 | 10000 | 127.44 |
| 24 | 56.52 | 31.69 | 62.69 | 10000 | 159.52 |
| 25 | 61.54 | 29.39 | 80.74 | 10000 | 123.86 |
| 26 | 14.05 | 14.6 | 10000 | 10000 | 1 |
| 27 | 72.85 | 56.5 | 18.24 | 132.12 | 7.24 |
| 28 | 65.26 | 34.42 | 58.14 | 1001.44 | 17.23 |
| 29 | 5.79 | 0.46 | 10000 | 10000 | 1 |
| 30 | 36.19 | 6.81 | 10000 | 10000 | 1 |
| 31 | 24.32 | 5.73 | 10000 | 10000 | 1 |
| 32 | 63.01 | 37.2 | 71.66 | 10000 | 139.55 |
| 33 | 78.86 | 56.04 | 11.67 | 71.91 | 6.16 |
| 34 | 58.63 | 30.58 | 47.01 | 10000 | 212.72 |
| 35 | 57.04 | 22.64 | 80.45 | 10000 | 124.30 |
| 36 | 63.89 | 27.42 | 32.26 | 10000 | 309.98 |
| 37 | 47.52 | 33.11 | 10000 | 10000 | 1 |
| 38 | 44.78 | 3.22 | 10000 | 10000 | 1 |
| 39 | 84.84 | 53.72 | 3.09 | 79.25 | 25.67 |
| 40 | 50.32 | 13.75 | 316.91 | 10000 | 31.55 |
| 41 | 36.84 | 40.98 | 10000 | 10000 | 1 |
| 42 | 28.68 | 9.32 | 10000 | 10000 | 1 |
| 43 | 78.53 | 59.31 | 11.93 | 83.41 | 6.99 |
| 44 | 86.58 | 77.54 | 6.22 | 24.56 | 3.95 |
| 45 | 29.48 | 9.72 | 10000 | 10000 | 1 |
| 46 | 63.33 | 45.08 | 33.67 | 10000 | 297.00 |
| 47 | 7.19 | 2.09 | 10000 | 10000 | 1 |
| 48 | 35.56 | 16.26 | 10000 | 10000 | 1 |
| 49 | 76.89 | 59.81 | 26.39 | 171.95 | 6.52 |
| 50 | 53.37 | 20.2 | 135.98 | 10000 | 73.54 |
| 51 | 26.69 | 0.85 | 10000 | 10000 | 1 |
| 52 | 44 | 13.73 | 10000 | 10000 | 1 |
| 53 | 47.22 | 9.06 | 10000 | 10000 | 1 |
| 54 | 80.55 | 56.79 | 6.7 | 58.57 | 8.74 |
| 59 | 72.79 | 25.96 | 11.88 | 10000 | 842 |
| 60 | 85.74 | 35.71 | 3.79 | 10000 | 2639 |
| 61 | 96.7 | 86.78 | 0.18 | 3.49 | 19 |
| 62 | 93.19 | 52.3 | 0.25 | 150.56 | 602 |
| 63 | 80.51 | 32.18 | 8.77 | 10000 | 1140 |
| 64 | 81.34 | 30.41 | 5.51 | 10000 | 1815 |
| 65 | 89.23 | 55.36 | 3.33 | 88.64 | 27 |
| 66 | 73.67 | 20.73 | 10.56 | 10000 | 947 |
| 67 | 96.16 | 72.94 | 0.4 | 20.41 | 51 |
| 68 | 93.25 | 66.92 | 0.57 | 19.22 | 34 |
| 69 | 85.26 | 44.11 | 6.23 | 10000 | 1605 |
| 70 | 81.43 | 35.06 | 8.6 | 10000 | 1163 |
| 71 | 81.58 | 32.2 | 8.07 | 10000 | 1239 |
| 72 | 95.81 | 75.43 | 0.61 | 25.89 | 42 |
| 73 | 79.77 | 21 | 10.99 | 10000 | 910 |
| 74 | 94.21 | 69.21 | 1.88 | 59.33 | 32 |
| 75 | 89.96 | 35.34 | 2.18 | 10000 | 4587 |
| 76 | 83.48 | 24.7 | 9.53 | 10000 | 1049 |
| 77 | 87.05 | 44.25 | 4.12 | 10000 | 2427 |
| 78 | 85.76 | 37.44 | 5.1 | 10000 | 1961 |
| 79 | 70.65 | 21.85 | 12.41 | 10000 | 806 |
| 80 | 59.96 | 21.88 | 14.19 | 10000 | 705 |
| 81 | 77.43 | 28.2 | 10.26 | 10000 | 975 |
| 82 | 71.22 | 23.82 | 11.32 | 10000 | 883 |
| 83 | 91.82 | 73.31 | 2.3 | 20.23 | 9 |
| 84 | 89.52 | 61.91 | 4.23 | 46.49 | 11 |
| 85 | 90.88 | 41.92 | 8.19 | 10000 | 1220 |
| 86 | 70.54 | 53.74 | 22.12 | 10000 | 452 |
| 87 | 87.71 | 22.46 | 2.79 | 10000 | 3583 |
| 88 | 78.43 | 20.16 | 7.76 | 10000 | 1289 |
| 89 | 82.14 | 47.26 | 3.8 | 10000 | 2634 |
| 90 | 76.46 | 34.83 | 8.55 | 10000 | 1170 |
| 91 | 79.84 | 41.81 | 4.52 | 10000 | 2212 |
| 92 | 80.82 | 50.24 | 4.81 | 92.52 | 19 |
| 93 | 69.83 | 33.84 | 21.11 | 10000 | 474 |
| 94 | 87.32 | 33.01 | 2.24 | 10000 | 4454 |
| 95 | 70.65 | 21.85 | 12.41 | 10000 | 806 |
| 96 | 87.06 | 60.02 | 4.3 | 39.61 | 9 |
| 97 | 86.62 | 65.8 | 3.14 | 20.75 | 7 |
| 98 | 86.73 | 58.08 | 5.55 | 57.95 | 10 |
| 99 | 57.29 | 46.88 | 48.11 | 10000 | 208 |
| 100 | 92.36 | 66.38 | 3.4 | 114.65 | 34 |
| 101 | 70.96 | 60.21 | 19.52 | 114.38 | 6 |
| 102 | 77.72 | 54.36 | 26.25 | 294.55 | 11 |
| 103 | 87.37 | 68.39 | 15.71 | 129.11 | 8 |
| 104 | 81.85 | 49.98 | 7.3 | 10000 | 1370 |
| 105 | 92.2 | 73.1 | 4.98 | 57.52 | 12 |
| 106 | 78.26 | 59.4 | 4.91 | 32.25 | 7 |
| 107 | 58.81 | 29.42 | 41.34 | 10000 | 242 |
| 108 | 66.54 | 52.47 | 23.37 | 179.02 | 8 |
| 109 | 82.73 | 67.11 | 76.89 | 497.55 | 6 |
| 110 | 80.85 | 56.88 | 16.7 | 272.95 | 16 |
| 111 | 81.44 | 57.54 | 11.11 | 210.29 | 19 |
| 112 | 92.01 | 87.71 | 4.19 | 20.77 | 5 |
| 113 | 83.56 | 63.38 | 9.76 | 87.36 | 9 |
| 114 | 78.63 | 49.85 | 9.94 | 10000 | 1006 |
| 115 | 77.62 | 45.22 | 10.68 | 10000 | 937 |
| 116 | 88.59 | 75.81 | 7.97 | 57.42 | 7 |
| 117 | 82.12 | 62.38 | 16.65 | 152.61 | 9 |
| 118 | 84.05 | 76.49 | 8.85 | 35.52 | 4 |
| 119 | 89.19 | 79.42 | 3.52 | 20.29 | 6 |
| 120 | 78.43 | 50.03 | 7.6 | 10000 | 1316 |
| 121 | 74.28 | 29.31 | 14.9 | 10000 | 671 |
| 122 | 73.6 | 34.8 | 9.13 | 10000 | 1095 |
| 123 | 66.82 | 35.05 | 68.06 | 10000 | 147 |
| 124 | 75.86 | 25.15 | 11.34 | 10000 | 882 |
| 125 | 81.97 | 62.54 | 6.01 | 36.95 | 6 |
| 126 | 80.91 | 32.23 | 32.84 | 10000 | 304 |
| 127 | 64.21 | 56.08 | 32.04 | 10000 | 312 |
| 128 | 71.59 | 39.1 | 11.12 | 10000 | 899 |
| 129 | 78.2 | 60.98 | 20.33 | 124.78 | 6 |
| 130 | 96.41 | 84.4 | 0.31 | 8.72 | 28 |
| 131 | 71.31 | 38.92 | 29.39 | 10000 | 340 |
| 132 | 71.58 | 14.97 | 10.16 | 10000 | 984 |
| 133 | 90.47 | 74.85 | 2.5 | 20.5 | 8 |
| 134 | 87.11 | 31.1 | 8.79 | 10000 | 1138 |
| 135 | 74.24 | 61.9 | 20.65 | 191.35 | 9 |
| 136 | 94.02 | 84.19 | 2.1 | 17.81 | 8 |
| 137 | 66.33 | 44.91 | 44.42 | 10000 | 225 |
| 138 | 89.02 | 67.08 | 4.09 | 38.27 | 9 |
| 139 | 89.15 | 53.99 | 4.55 | 166.83 | 37 |
| 140 | 86.93 | 31.22 | 3.79 | 10000 | 2641 |
| 141 | 86.99 | 71.54 | 6.59 | 34.91 | 5 |
| 142 | 69.9 | 59.96 | 24.51 | 84.77 | 3 |
| 143 | 86.51 | 66.72 | 13.28 | 160.89 | 12 |
| 144 | 93.65 | 80.68 | 1.36 | 13.38 | 10 |
| 145 | 92.79 | 84.55 | 1.69 | 9.67 | 6 |
| 146 | 83.03 | 72.85 | 5.57 | 21.93 | 4 |
| 147 | 76.75 | 64.49 | 13.74 | 47.26 | 3 |
| 148 | 82.97 | 41.4 | 6.84 | 10000 | 1462 |
| 149 | 84.88 | 41.01 | 7.34 | 10000 | 1362 |
| 150 | 73.9 | 50.62 | 22.64 | 10000 | 442 |
| 151 | 92.33 | 59.6 | 3.57 | 156.85 | 44 |
| 152 | 66.51 | 45.41 | 68.22 | 10000 | 147 |
| 153 | 92.18 | 86.17 | 10.27 | 47.49 | 5 |
| 154 | 87.71 | 65.63 | 2.87 | 22.28 | 8 |
| 155 | 74.95 | 51.52 | 13.7 | 10000 | 730 |
| 156 | 63.24 | 35.98 | 38.49 | 10000 | 260 |
| 157 | 91.82 | 82.62 | 1.56 | 8.12 | 5 |
| 158 | 92.01 | 88.48 | 0.44 | 1.36 | 3 |
| 159 | 89.5 | 82.15 | 0.38 | 1.45 | 4 |
| 160 | 87.71 | 71.36 | 12.04 | 103.52 | 9 |
| 161 | 70.84 | 32.39 | 31.92 | 10000 | 313 |
| 162 | 84.71 | 63.92 | 2.62 | 25.92 | 10 |
| 163 | 61.96 | 49.32 | 40.81 | 10000 | 245 |
| 164 | 85.04 | 86.62 | 2.16 | 3.77 | 2 |
| 165 | 57.95 | 34 | 45.84 | 1001.44 | 22 |
| 166 | 65.83 | 35.21 | 24.21 | 316.91 | 13 |
| 167 | 67.5 | 23.66 | 40.13 | 659.18 | 16 |
| 168 | 90.6 | 77.29 | 18.41 | 113.31 | 6 |
| 169 | 86.6 | 62 | 6.28 | 104.47 | 17 |
| 170 | 59.18 | 48.47 | 38.33 | 10000 | 261 |
| 171 | 69.5 | 41.42 | 28.52 | 1001.44 | 35 |
| 172 | 69.76 | 29.36 | 54.85 | 10000 | 182 |
| 173 | 61.98 | 39.84 | 44.87 | 10000 | 223 |
| 174 | 68.17 | 50.75 | 37.23 | 10000 | 269 |
| 175 | 60.18 | 35.58 | 29.84 | 10000 | 335 |
| 176 | 76.81 | 53.03 | 22.28 | 10000 | 449 |
| 177 | 71.51 | 54.49 | 3.53 | 21.64 | 6 |
| 178 | 76.2 | 62.24 | 10.11 | 70.26 | 7 |
| 179 | 83.42 | 57.74 | 17.94 | 211.3 | 12 |
| 180 | 77.23 | 46.22 | 13.26 | 10000 | 754 |
| 181 | 72.35 | 42.28 | 19.58 | 316.91 | 16 |
| 182 | 82.55 | 57.09 | 2.76 | 62.63 | 23 |
| 183 | 80.66 | 56.25 | 6.43 | 248.73 | 39 |
| 184 | 91.34 | 80.66 | 4.88 | 37.57 | 8 |
| 185 | 88.8 | 58.54 | 7.13 | 142.77 | 20 |
| 186 | 83.16 | 43 | 2.12 | 10000 | 4811 |
| 187 | 94.44 | 73.08 | 1.3 | 34.49 | 27 |
| 188 | 90.59 | 57.84 | 1.71 | 244.9 | 143 |
| 189 | 93.05 | 81.78 | 1.7 | 15.57 | 9 |
| 190 | 89.21 | 58.83 | 2.72 | 81.9 | 30 |
| 191 | 79.63 | 54.65 | 6.43 | 10000 | 1556 |
| 192 | 88.83 | 46.37 | 4.58 | 128.11 | 28 |
| 193 | 94.88 | 84.22 | 1.24 | 13.24 | 11 |
| 194 | 88.14 | 70.32 | 3.24 | 31.35 | 10 |
| 195 | 70.02 | 20.3 | 13.75 | 10000 | 727 |
| 196 | 74 | 28 | 14 | 10000 | 714 |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having a structure represented by Formula (IA): 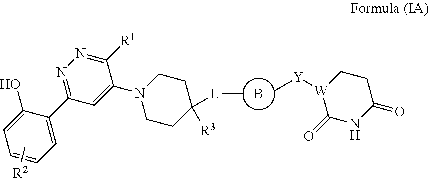
or a stereoisomer, pharmaceutically acceptable salt thereof, wherein:
R¹ is hydrogen or C₁₋₆ alkyl optionally substituted with 1 to 3 R^{a};
R² is hydrogen, halo, hydroxyl, —O—R^{e}, C₁₋₆ alkyl optionally substituted with 1 to 3 R^{a};
R³ is hydrogen, —CN, C₁₋₆ alkyl, C₆₋₁₂ aryl, C₃₋₁₂ cycloalkyl, 5-12 membered heteroaryl, each being optionally substituted with 1 to 3 R^{d}, or —O—R^{e};
L comprises up to 8 linker segments represented by -L₁-L₂-L₃-L₄-L₅-L₆-L₇-L₈-, each L₁, L₂, L₃, L₄, L₅, L₆, L₇, or L₈, being independently:
i) C₃₋₁₂ cycloalkyl optionally substituted with 1-3 R^{b};
ii) C₆₋₁₂ aryl optionally substituted with 1-3 R^{b};
iii) 4-12 membered heterocyclyl optionally substituted with 1-3 R^{b};
iv) 5-12 membered heteroaryl optionally substituted with 1-3 R^{b};
v) direct bond;
vi) C₁₋₁₂ alkylene chain optionally substituted with 1-3 R^{b}; or
vii) —(CH₂)ₘ—C(O)—, —(CH₂)ₘ—C(O)O—, —(CH₂)ₘ—O—, —(CH₂)ₘ—N(R^{c})—, —(CH₂)ₘ—S—, —(CH₂)ₘ—C(S)—, —(CH₂)ₘ—C(S)—O—, —(CH₂)ₘ—S(O)₂—, —(CH₂)ₘ—S(O)═N—, —(CH₂)ₘ—S(O)₂NH—, —(CH₂)ₘ—C(O)—N(R^{c})—, —C(O)—N(R^{c})—(CH₂)ₘ—, —(CH₂)ₘ—O—C(O)—N(R^{c})—, —(CH₂)ₘ—O—C(O)—O—, or —NH—(CH₂)ₘ—C(O)—, wherein m is 0, 1, 2, 3, 4, 5 or 6;
each R^{a} is independently halo, or —O—R^{c};
each R^{b} is independently oxo, imino, sulfoximino, halo, nitro, —CN, C₁₋₆ alkyl, C₂₋₆ alkenyl, C₃₋₁₅ cycloalkyl, C₁₋₈ haloalkyl, C₆₋₁₂ aryl, 5-12 membered heteroaryl, 4-12 membered heterocyclyl, —O—R^{c}, —C(O)—R^{c}, —C(O)O—R^{c}, —C(O)—N(R^{c})(R), —N(R^{c})(R^{c}), —N(R^{c})C(O)—R^{c}, —N(R^{c})C(O)O—R^{c}, —N(R^{c})C(O)N(R^{c})(R^{c}), —N(R^{c})S(O)₂(R^{c}), —NR^{c}S(O)₂N(R^{c})(R^{c}), —N(R^{c})S(O)₂O(R^{c}), —OC(O)R^{c}, —OC(O)—N(R^{c})(R^{c}), —Si(R^{c})₃, —S—R^{c}, —S(O)R^{c}, —S(O)(NH)R^{c}, —S(O)₂R^{c} or —S(O)₂N(R^{c})(R^{c}), wherein each of C₁₋₆ alkyl, C₂₋₆ alkenyl, C₃₋₁₅ cycloalkyl, C₁₋₈ haloalkyl, C₆₋₁₂ aryl, 5-12 membered heteroaryl, and 4-12 membered heterocyclyl may be optionally substituted with 1 to 3 R^{d};
each R^{c} is independently hydrogen, C₁₋₆ alkyl, C₃₋₆ cycloalkyl, C₆₋₁₂ aryl or C₁₋₆ haloalkyl, wherein C₁₋₆ alkyl is optionally substituted with C₁₋₃ alkoxy;
each R^{d} is independently halo, —CN, —O—R^{c}, C₁₋₆ alkyl, C₆₋₁₂ aryl or C₁₋₆ haloalkyl;
R^{c} is C₁₋₆ alkyl, C₃₋₁₂ cycloalkyl, C₆₋₁₂ aryl, 5-12 membered heteroaryl, each being optionally substituted with C₁₋₆ alkyl or halo;
W is —C(R^{g})— or —N—;
Y is direct bond, C₁₋₄ alkylene chain, —C(O)—, —C(O)O—, —O—, —N(R^{g})—, —S——C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R³;
R^{g} is hydrogen or C₁₋₆ alkyl; and
each R^{j} is independently halo, oxo, —CN, —O—R^{c}, C₁₋₆ alkyl, or C₁₋₆ haloalkyl.

2. The compound of claim 1, wherein each L₁, L₂, L₃, L₄, L₅, L₆, L₇, or L₈ is independently:
i) a bivalent ring moiety selected from the group consisting of: 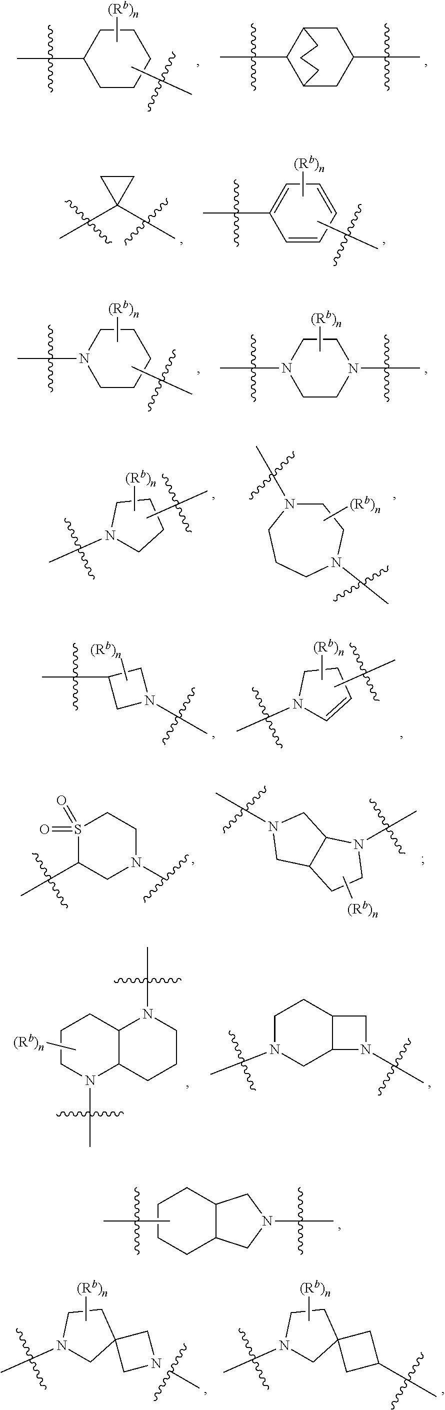 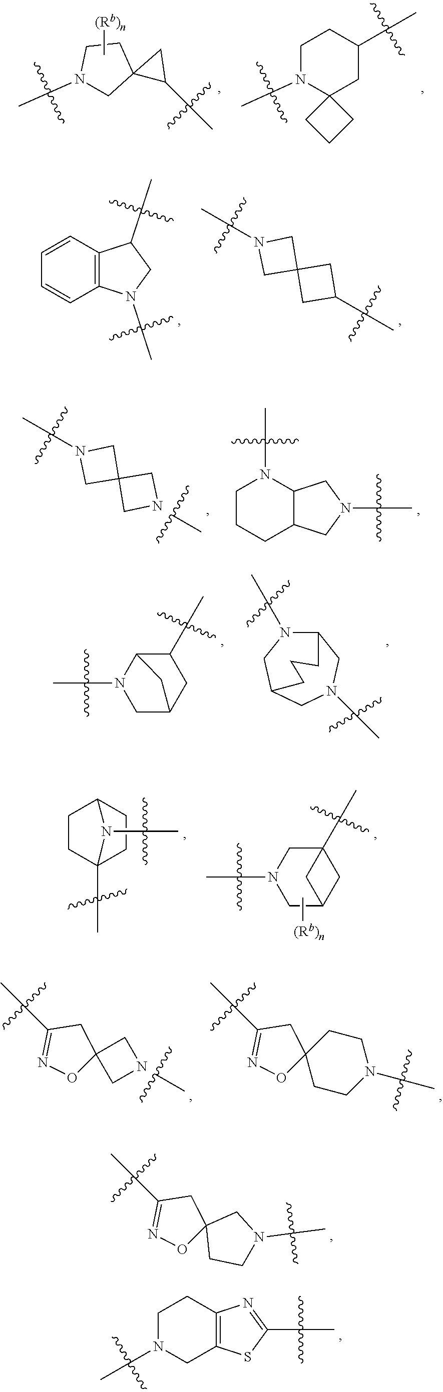 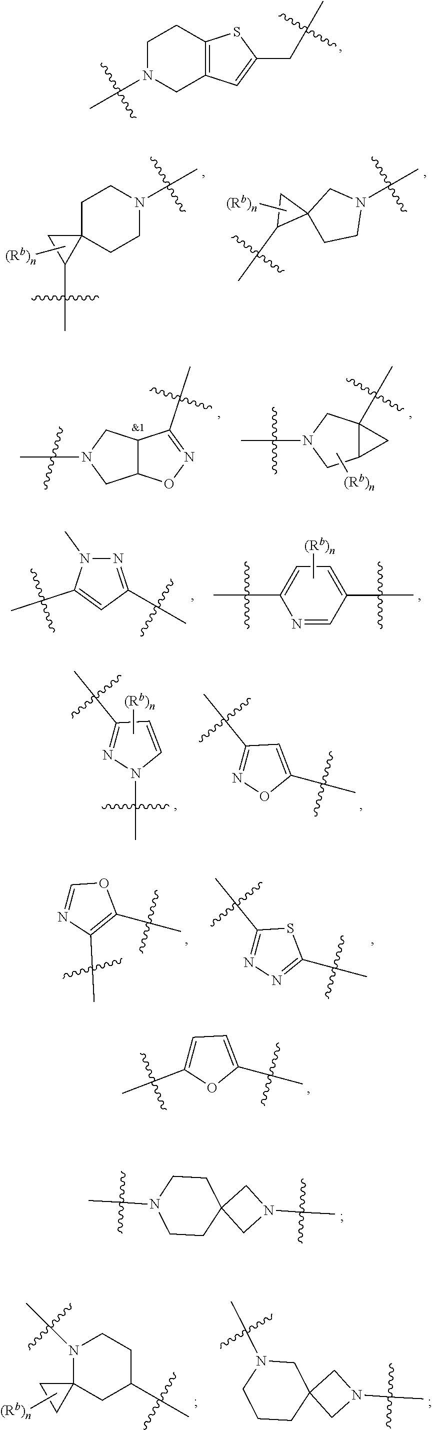 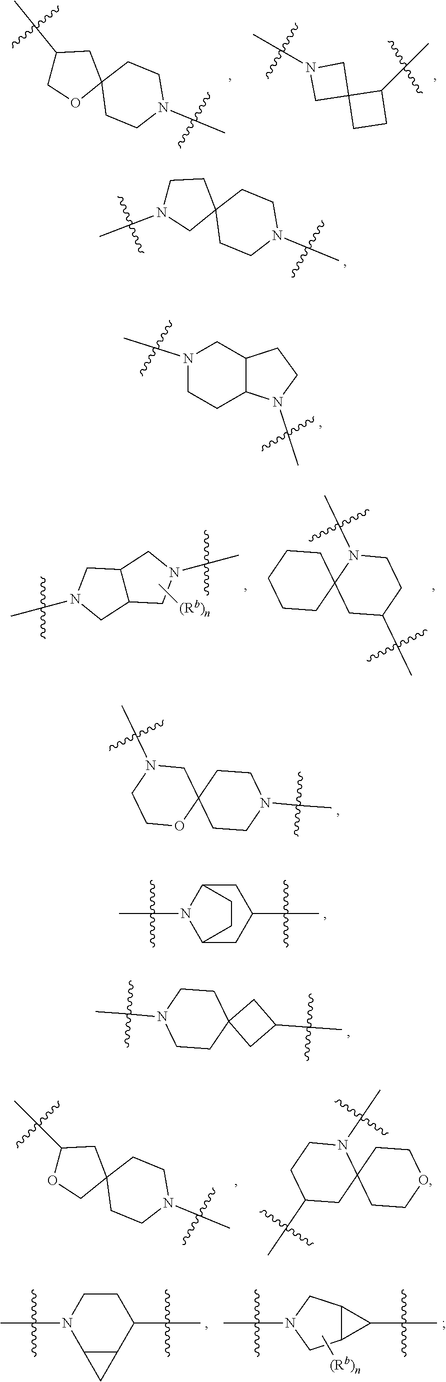
ii) direct bond;
iii) C₁₋₆ alkylene chain; or
iv) —C(O)—, —O—, —(CH₂)ₘ—C(O)—N(R^{c})—, —(CH₂)ₘ—N(R^{c})—, —C(O)—N(R^{c})—(CH₂)ₘ—, or —NH—(CH₂)ₘ—C(O)—, —(CH₂)ₘ—S(O)₂NH—, m being 0, 1, 2 or 3;
wherein,
n is 0, 1, or 2;
R^{b} is halo, —O—R, —CN, C₁₋₆ alkyl, or C₁₋₆ haloalkyl; and
R^{c} is hydrogen, C₁₋₆ alkyl, C₃₋₆ cycloalkyl, phenyl, or C₁₋₆ haloalkyl, wherein C₁₋₆ alkyl is optionally substituted with C₁₋₃ alkoxy.

3. The compound of claim 1, wherein,
Y is direct bond, —NHC(O)—, or —NH—; and
B ring is 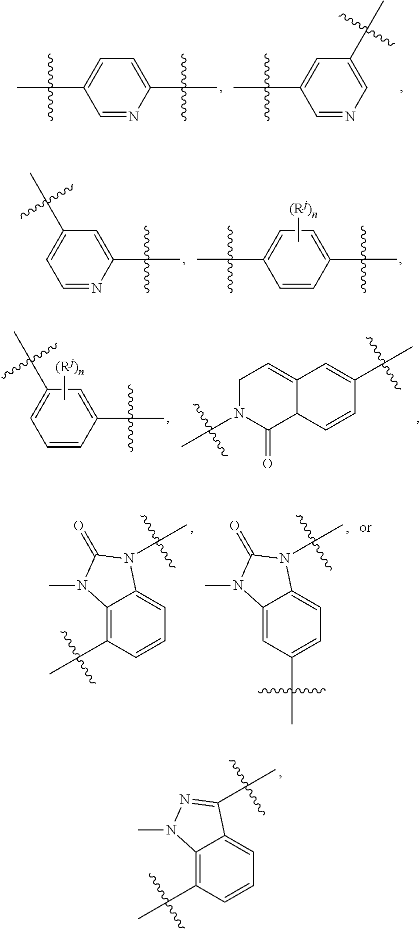
wherein n is 0, 1 or 2, R is halo, —CN, —O—R^{c}, C₁₋₆ alkyl, or C₁₋₆ haloalkyl.

4. The compound of claim 1, wherein R³ is:
phenyl optionally substituted with one or more C₁₋₆ alkyl, C₁₋₆ alkoxy, or halo;
benzyl optionally substituted with one or more C₁₋₆ alkyl or halo;
pyridinyl optionally substituted with one or more C₁₋₆ alkyl;
CN;
C₃₋₆ cyclopropyl;
pyrazolyl optionally substituted with one or more C₁₋₆ alkyl;
tetrahydropyranyl,
1,2-oxazolyl optionally substituted with one or more C₁₋₆ alkyl;
pyrazolo[1,5-a]pyridinyl; or
—O—R^{e}, wherein R^{c} is phenyl optionally substituted with C₁₋₆ alkyl or halo, C₃₋₆ cycloalkyl, C₁₋₆ alkyl, or pyrazolyl optionally substituted with C₁₋₆ alkyl.

5. The compound of claim 4, wherein R³ is phenyl, benzyl, 2-pyridinyl, 3-pyridinyl, 4-pyridinyl, CN, cyclopropyl, 1-methyl-1H-pyrazol-3yl, 3-methylphenyl, 2-methylphenyl, 3-chlorophenyl, 3,4-dichlorophenyl, 2-methoxyphenyl, 3-methoxyphenyl, 2-fluorophenyl, 4-fluorophenyl, tetrahydropyran-4yl, 3-methyl-1H-pyrazol-1yl, 4-methyl-1H-pyrazol-1yl, 5-methyl-1H-pyrazol-1yl, 1-methyl-1H-pyrazol-3yl, 1-methyl-1H-pyrazol-4yl, 1-methyl-1H-pyrazol-5yl, pyrazolo[1,5-a]pyridin-2-yl, 5-methyl-1,2-oxazol-3-yl, 5-isopropyl-1,2-oxazol-3-yl, 3-methyl-1,2-oxazol-5-yl, or —O—R^{e}, wherein R^{e} is phenyl, 2-chlorophenyl, 2-methylphenyl, cyclopropyl, cyclohexyl, methyl, or 1-methyl-1H-pyrazol-3yl.

6. The compound of claim 1, wherein R¹ is hydrogen and R² is hydrogen.

7. The compound of claim 1, wherein R¹ is hydrogen, R² is hydrogen, Y is direct bond, R³ is —O—R^{e}, W is CH, and the compound has a structure represented by Formula (IA1): 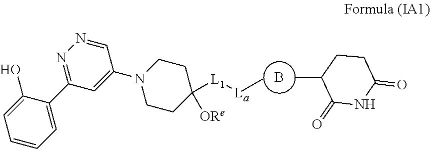
wherein,
B ring is 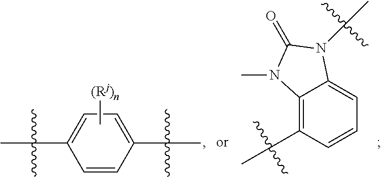
n is 0, 1 or 2;
R^{j} is halo, —CN, —O—R^{c}, C₁₋₆ alkyl, or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl or halo;
R^{c} is hydrogen or C₁₋₃ alkyl;
L₁ is —C(O)—, or —C(O)N(R^{c})—; and
Lₐ is

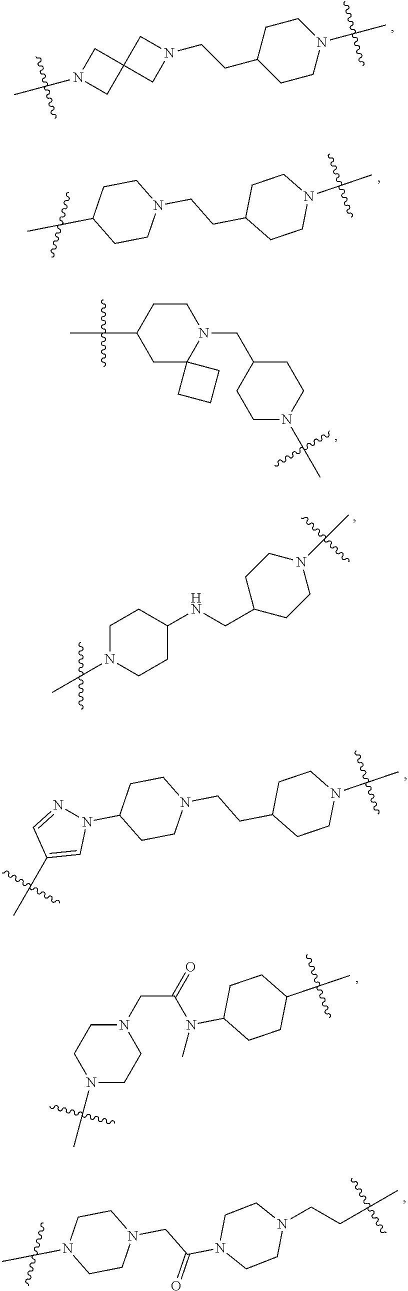

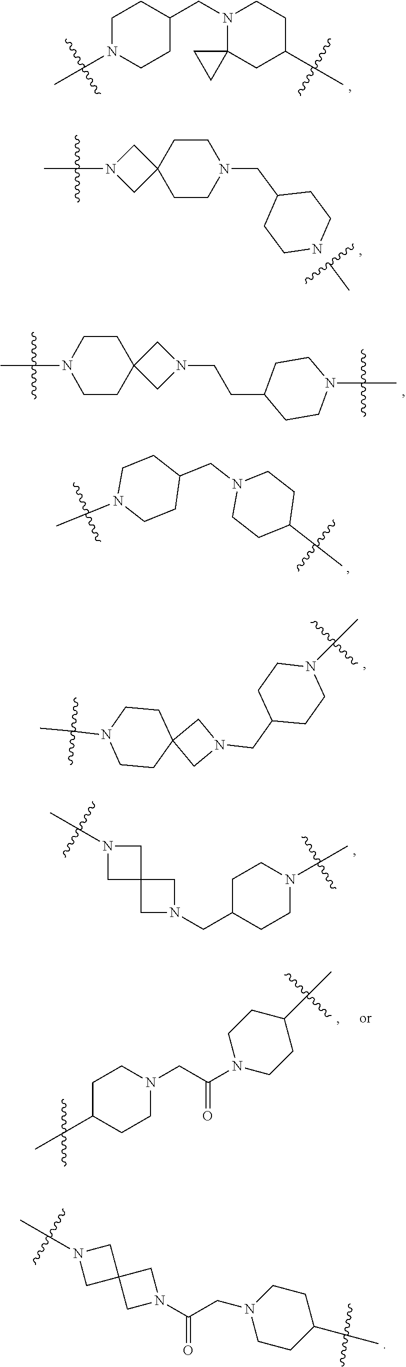

8. The compound of claim 7, wherein B is 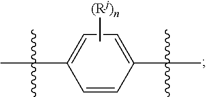
n is 0 or 1;
R^{j} is fluoro or chloro;
R^{e} is C₁₋₆ alkyl, C₃₋₁₂ cycloalkyl, C₆₋₁₂ aryl, 5-12 membered heteroaryl, each being optionally substituted with C₁₋₆ alkyl or halo;
L¹ is —C(O)N(R^{c})— where R^{c} is methyl or ethyl; and
Lₐ is

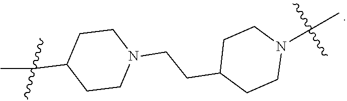 [SMILES: *C1CCN(CCC2CCN(*)CC2)CC1]

9. The compound of claim 8, wherein R^{e} is C₁₋₃ alkyl.

10. The compound of claim 7, wherein R^{e} is methyl, phenyl, cyclopropyl, cyclohexyl, 1-methyl-1H-pyrazol-3yl or phenyl substituted with chloro or methyl.

11. The compound of claim 7, wherein B ring is 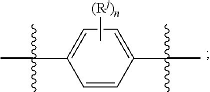
n is 0, 1 or 2;
R^{j} is fluoro or chloro;
R^{e} is methyl, ethyl, or propyl;
R^{c} is hydrogen, methyl, or ethyl;
L₁ is —C(O)—, or —C(O)N(R^{c})—; and
Lₐ is

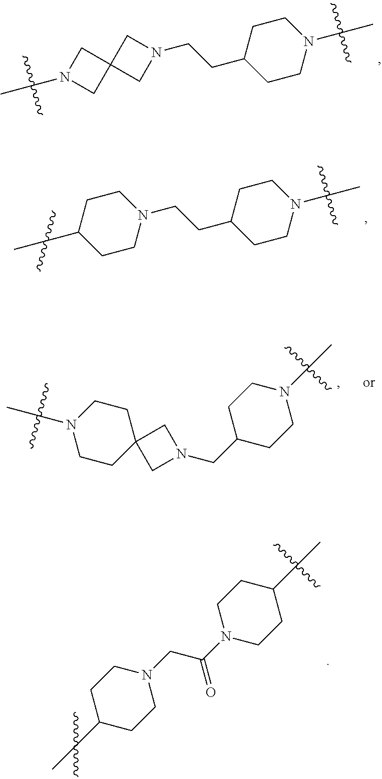 [SMILES: *N1CCC(CCN2CC3(CN(*)C3)C2)CC1 ; *C1CCN(CCC2CCN(*)CC2)CC1 ; *N1CCC(CN2CC3(CCN(*)CC3)C2)CC1 ; *C1CCN(CC(=O)N2CCC(*)CC2)CC1]

12. The compound of claim 1, wherein R¹ and R² are hydrogen, W is CH, and the compound has a structure represented by Formula (IA2): 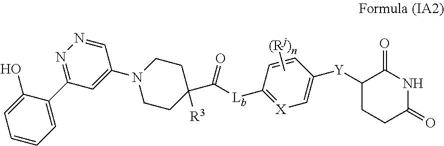
wherein,
n is 0, 1, or 2;
Y is direct bond, —NHC(O)—, or —NH—;
X is N or CH;
R^{j} is C₁₋₃alkyl or halo;
R³ is C₆₋₁₂ aryl, C₃₋₁₂ cycloalkyl, 5-12 membered heteroaryl, each being optionally substituted with 1 to 3 R^{d}, or —O—R^{e};
R^{c} is hydrogen or C₁₋₆ methyl;
each R^{d} is independently halo, —CN, —O—R^{c}, C₁₋₆ alkyl, C₆₋₁₂ aryl or C₁₋₆ haloalkyl; and
R^{e} is C₁₋₆ alkyl, C₃₋₁₂ cycloalkyl, C₆₋₁₂ aryl, 5-12 membered heteroaryl, each being optionally substituted with C₁₋₆ alkyl or halo; and
L_{b} is

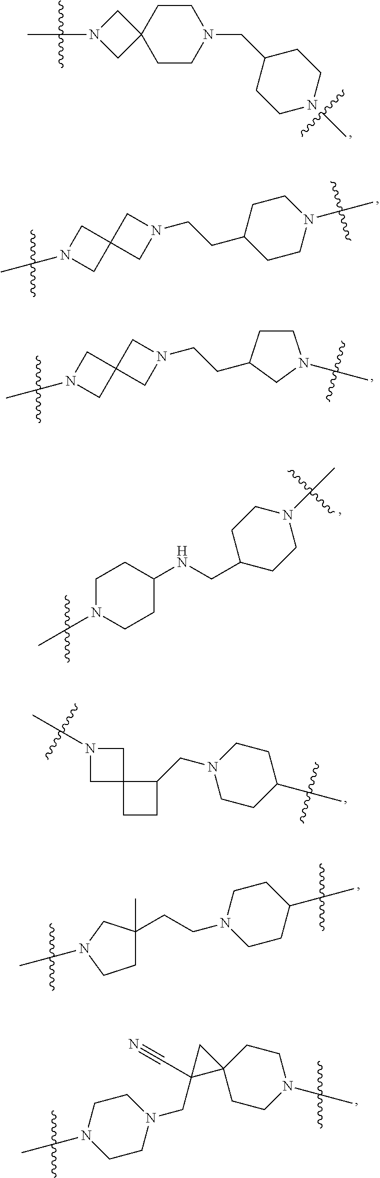

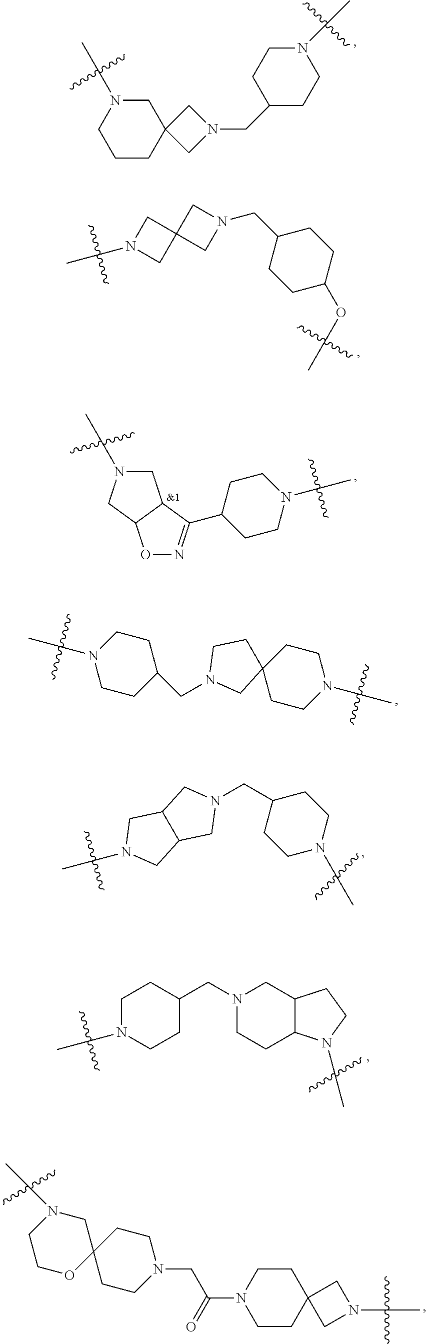

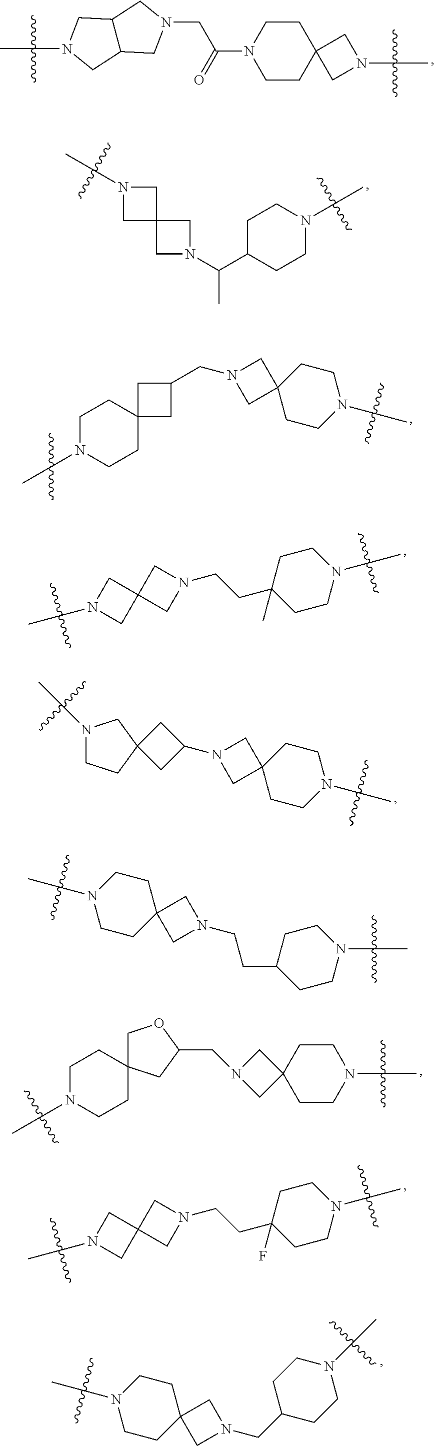

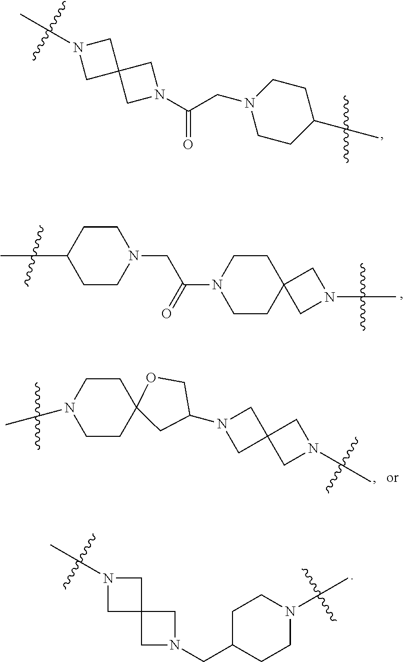 [SMILES: *C1CCN(CC(=O)N2CC3(CN(*)C3)C2)CC1 ; *C1CCN(CC(=O)N2CCC3(CC2)CN(*)C3)CC1 ; *N1CCC2(CC1)CC(N1CC3(CN(*)C3)C1)CO2 ; *N1CCC(CN2CC3(CN(*)C3)C2)CC1]

13. The compound of claim 12, wherein R³ is phenyl, 2-fluorophenyl, 5-methyl-1,2-oxazol-5-yl, 5-isopropyl-1,2-oxazol-5-yl, pyrazolo[1,5-a]pyridinyl, —O—R^{e}, wherein R^{e} is phenyl, 2-chlorophenyl, 2-methylphenyl, 1-methyl-1H-pyrazol-3yl, cyclohexyl, or methyl.

14. The compound of claim 1, wherein R¹ and R² are hydrogen, W is CH, and the compound has a structure represented by Formula (IA3): 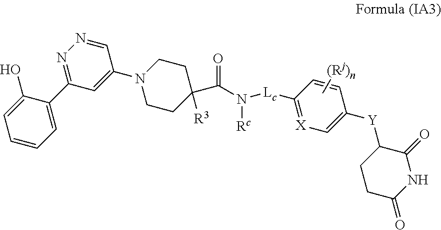
wherein,
n is 0, 1, or 2;
Y is direct bond, —NHC(O)—, or —NH—;
X is N or CH;
R³ is C₆₋₁₂ aryl, C₃₋₁₂ cycloalkyl, 5-12 membered heteroaryl, each being optionally substituted with 1 to 3 R^{d}, or —O—R^{e};
R^{c} is hydrogen, C₁₋₃alkyl, or C₃₋₆ cycloalkyl;
each R^{d} is independently halo, —CN, —O—R^{c}, C₁₋₆ alkyl, C₆₋₁₂ aryl or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl or halo;
R^{j} is C₁₋₃alkyl or halo; and
L_{c} is

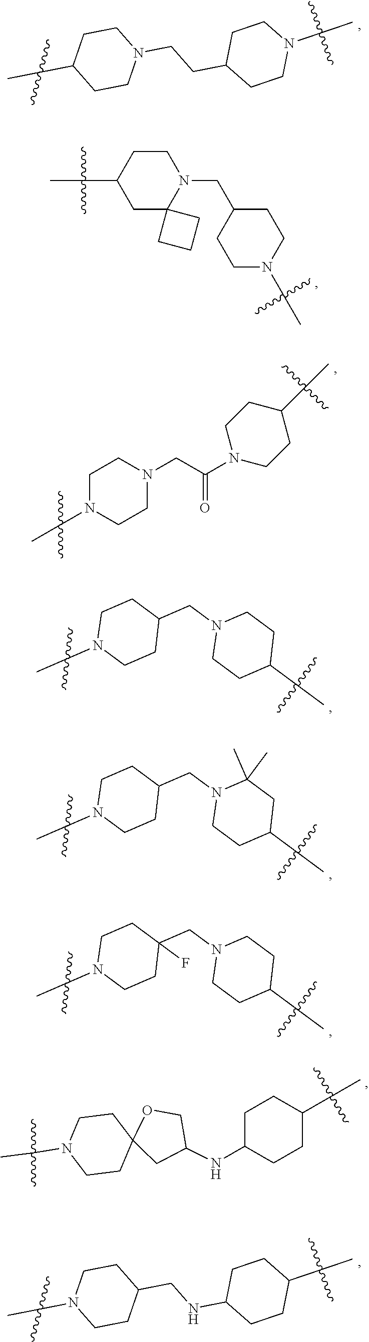

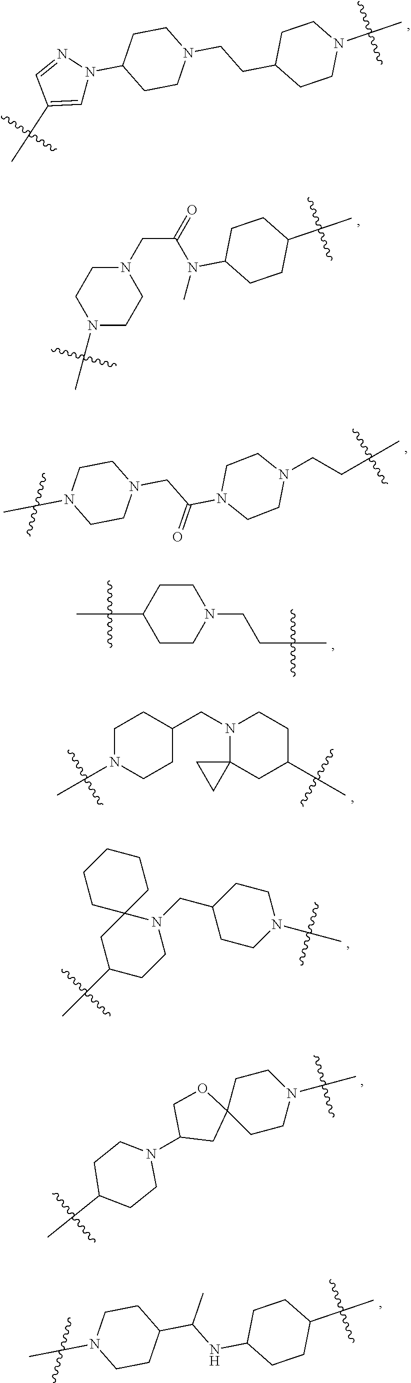

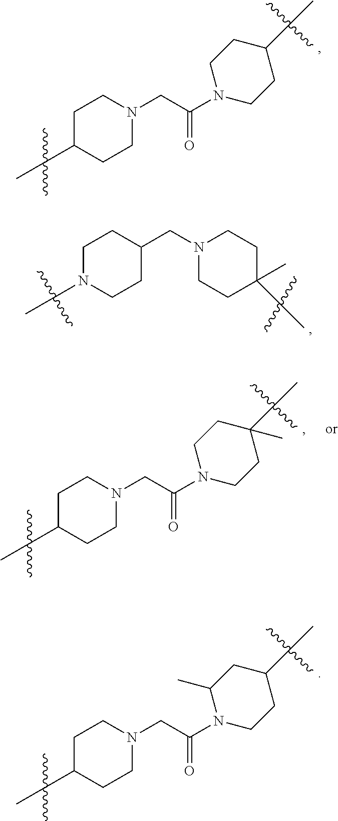 [SMILES: *C1CCN(CC(=O)N2CCC(*)CC2)CC1 ; *N1CCC(CN2CCC(*)(C)CC2)CC1 ; *C1CCN(CC(=O)N2CCC(*)(C)CC2)CC1 ; *C1CCN(CC(=O)N2CCC(*)CC2C)CC1]

15. The compound of claim 14, wherein R³ is phenyl, 2-pyridinyl, 4-pyridinyl, 2-fluorophenyl, 4-fluorophenyl, 3-methyl-1H-pyrazol-1yl, 4-methyl-1H-pyrazol-1yl, 5-methyl-1H-pyrazol-1yl, 1-methyl-1H-pyrazol-4yl, 1-methyl-1H-pyrazol-5yl, tetrahydropyran-4yl, 3-methyl-1,2-oxazol-5-yl, 5-isopropyl-1,2-oxazol-3-yl, pyrazolo[1,5-a]pyridinyl, —O—R^{e}, wherein R^{e} is phenyl, 2-chlorophenyl, 2-methylphenyl, 1-methyl-1H-pyrazol-3yl, cyclopropyl, cyclohexyl, or methyl.

16. The compound of claim 1, wherein R¹ and R² are hydrogen, W is CH, Y is direct bond, R³ is phenyl, and the compound has a structure represented by Formula (IA4): 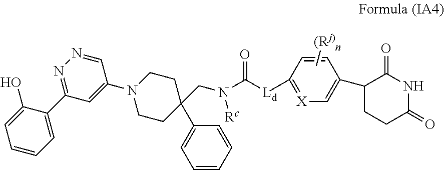
wherein,
n is 0, 1, or 2;
X is N or CH;
R^{e} is hydrogen or C₁₋₃alkyl optionally substituted with C₁₋₃ alkoxy;
R^{j} is C₁₋₃alkyl or halo; and
L^{d} is

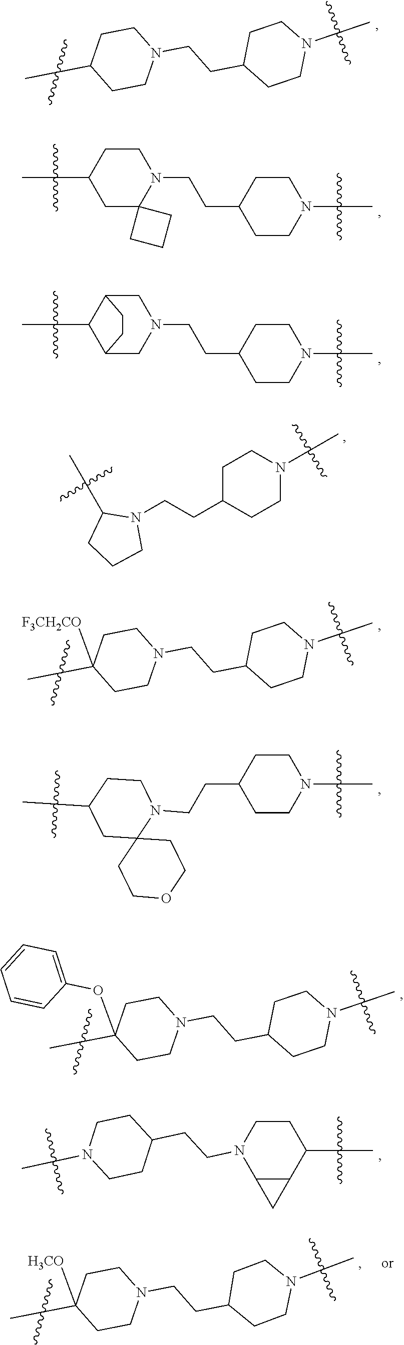

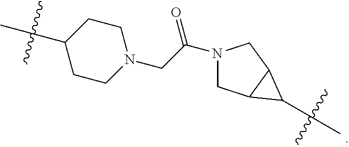 [SMILES: *C1CCN(CC(=O)N2CC3C(*)C3C2)CC1]

17. The compound of claim 1, wherein R¹ and R² are hydrogen, W is CH, Y is direct bond, R³ is phenyl, and the compound has a structure represented by Formula (IA5) 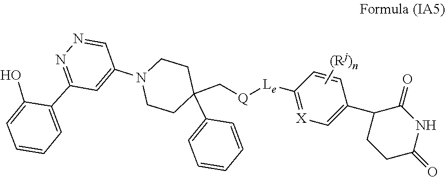
wherein,
n is 0, 1, or 2;
X is N or CH;
Q is direct bond, —N(R^{e})— or —NHS(O)₂—,
R^{e} is hydrogen or C₁₋₃alkyl;
R^{j} is C₁₋₃alkyl or halo; and
Lₑ is

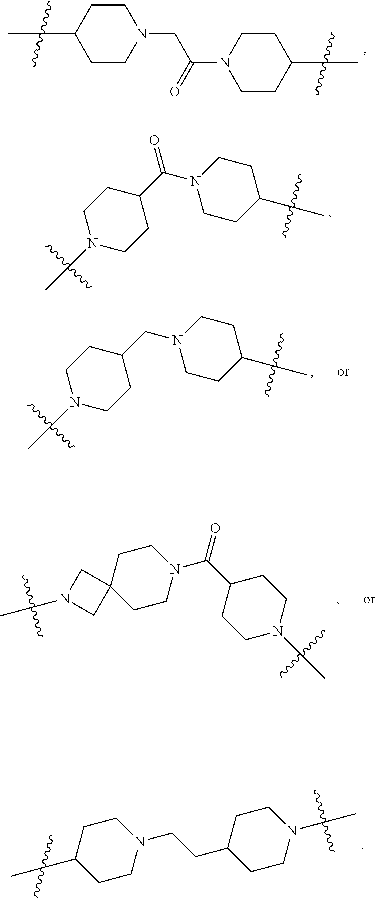 [SMILES: *C1CCN(CC(=O)N2CCC(*)CC2)CC1 ; *C1CCN(C(=O)C2CCN(*)CC2)CC1 ; *C1CCN(CC2CCN(*)CC2)CC1 ; *N1CCC(C(=O)N2CCC3(CC2)CN(*)C3)CC1 ; *C1CCN(CCC2CCN(*)CC2)CC1]

18. The compound of claim 1, wherein R¹ and R² are hydrogen, W is CH, Y is direct bond, and the compound has a structure represented by Formula (IA6): 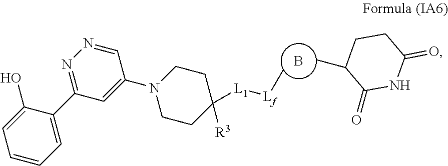
wherein,
B is 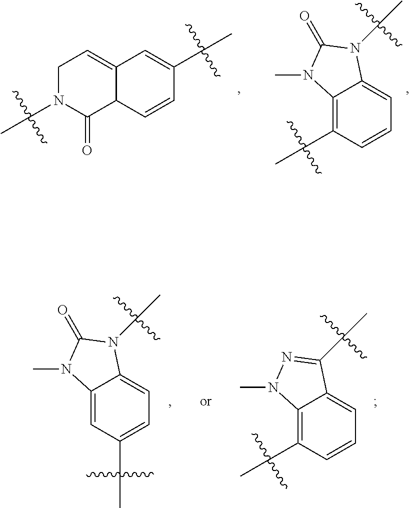 [SMILES: *C1=CC2=CCN(*)C(=O)C2C=C1 ; *c1cccc2c1n(C)c(=O)n2* ; *c1ccc2c(c1)n(C)c(=O)n2* ; *c1nn(C)c2c(*)cccc12]
R³ is C₆₋₁₂ aryl, C₃₋₁₂ cycloalkyl, 5-12 membered heteroaryl, each being optionally substituted with 1 to 3 R^{d}, or —O—R^{e};
R^{c} is hydrogen or C₁₋₆ alkyl;
each R^{d} is independently halo, —CN, —O—R^{c}, C₁₋₆ alkyl, C₆₋₁₂ aryl or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl or halo;
L₁ is —C(O)—, —C(O)—N(R^{c})—(CH₂)—, or —C(O)—N(R^{c})—; and
L_{f} is

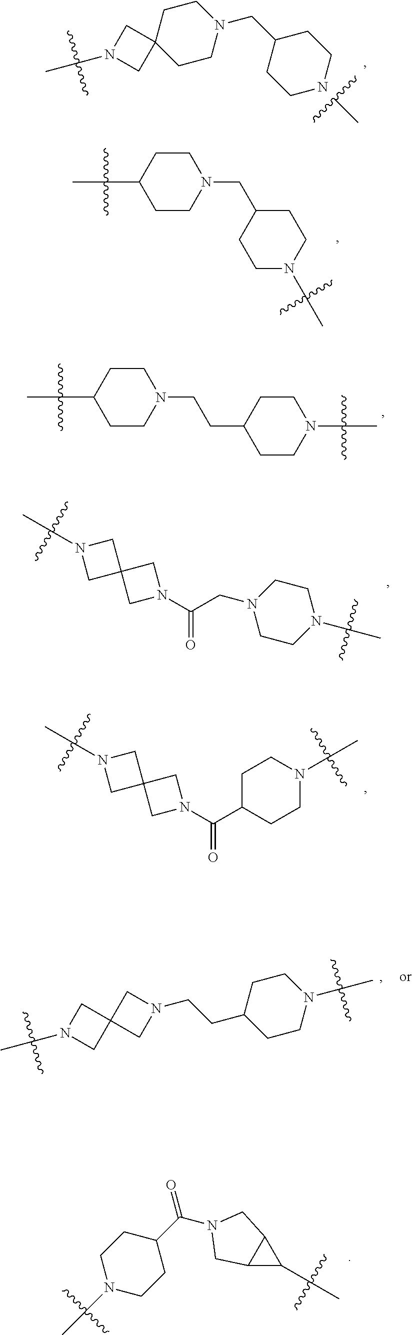

19. The compound of claim 18, wherein, R³ is phenyl, 1-methyl-1H-pyrazol-5yl, 5-methyl-1H-pyrazol-1yl, or —O—R^{c}, wherein R^{c} is 1-methyl-1H-pyrazol-3yl, or methyl.

20. A compound having a structure represented by Formula (I): 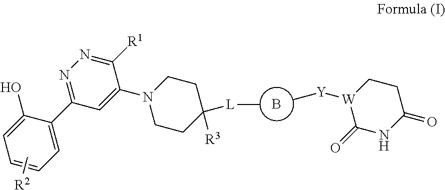
or a stereoisomer, pharmaceutically acceptable salt thereof, wherein:
R¹ is hydrogen or C₁₋₆ alkyl optionally substituted with 1 to 3 R^{a};
R² is hydrogen, halo, hydroxyl, —O—R^{c}, C₁₋₆ alkyl optionally substituted with 1 to 3 R^{a};
R³ is hydrogen, —CN, C₁₋₆ alkyl, C₆₋₁₂ aryl, C₃₋₁₂ cycloalkyl, or 5-12 membered heteroaryl, each being optionally substituted with 1 to 3 R^{d};
L comprises up to 8 linker segments represented by -L₁-L₂-L₃-L₄-L₅-L₆-L₇-L₈-, each L₁, L₂, L₃, L₄, L₅, L₆, L₇, or L₈, being independently:
i) C₃₋₁₂ cycloalkyl optionally substituted with 1-3 R^{b};
ii) C₆₋₁₂ aryl optionally substituted with 1-3 R^{b};
iii) 4-12 membered heterocyclyl optionally substituted with 1-3 R^{b};
iv) 5-12 membered heteroaryl optionally substituted with 1-3 R^{b};
v) direct bond;
vi) C₁₋₁₂ alkylene chain optionally substituted with 1-3 R^{b}; or
vii) —(CH₂)ₘ—C(O)—, —(CH₂)ₘ—C(O)O—, —(CH₂)ₘ—O—, —(CH₂)ₘ—N(R^{c})—, —(CH₂)ₘ—S—, —(CH₂)ₘ—C(S)—, —(CH₂)ₘ—C(S)—O—, —(CH₂)ₘ—S(O)₂—, —(CH₂)ₘ—S(O)═N—, —(CH₂)ₘ—S(O)₂NH—, —(CH₂)ₘ—C(O)—N(R^{c})—, —C(O)—N(R^{c})—(CH₂)ₘ—, —CH₂)ₘ—O—C(O)—N(R^{c})—, —(CH₂)ₘ—O—C(O)—O—, or —NH—(CH₂)ₘ—C(O)—, wherein m is 0, 1, 2, 3, 4, 5 or 6;
each R^{a} is independently halo, or —O—R^{c};
each R^{b} is independently oxo, imino, sulfoximino, halo, nitro, —CN, C₁₋₆ alkyl, C₂₋₆ alkenyl, C₃₋₁₅ cycloalkyl, C₁₋₈ haloalkyl, C₆₋₁₂ aryl, 5-12 membered heteroaryl, 4-12 membered heterocyclyl, —O—R, —C(O)—R^{c}, —C(O)O—R^{c}, —C(O)—N(R^{c})(R^{c}), —N(R^{c})(R^{c}), —N(R^{c})C(O)—R^{c}, —N(R^{c})C(O)O—R^{c}, —N(R^{c})C(O)N(R^{c})(R^{c}), —N(R^{c})S(O)₂(R^{c}), —NR^{c}S(O)₂N(R^{c})(R^{c}), —N(R^{c})S(O)₂O(R^{c}), —OC(O)R^{c}, —OC(O)—N(R^{c})(R^{c}), —Si(R^{c})₃, —S—R^{c}, —S(O)R^{c}, —S(O)(NH)R^{c}, —S(O)₂R^{c} or —S(O)₂N(R^{c})(R^{c}), wherein each of C₁₋₆ alkyl, C₂₋₆ alkenyl, C₃₋₁₅ cycloalkyl, C₁₋₈ haloalkyl, C₆₋₁₂ aryl, 5-12 membered heteroaryl, and 4-12 membered heterocyclyl may be optionally substituted with 1 to 3 R^{d};
each R^{c} is independently hydrogen, C₁₋₆ alkyl, or C₁₋₆ haloalkyl;
each R^{d} is independently halo, —CN, —O—R^{c}, C₁₋₆ alkyl, C₆₋₁₂ aryl or C₁₋₆ haloalkyl;
W is —C(R^{g})— or —N—;
Y is direct bond, C₁₋₄ alkylene chain, —C(O)—, —C(O)O—, —O—, —N(R^{g})—, —S——C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R^{j};
R^{g} is hydrogen or C₁₋₆ alkyl; and
each R^{j} is independently halo, —CN, —O—R^{c}, C₁₋₆ alkyl, or C₁₋₆ haloalkyl.

21. The compound of claim 20, wherein,
Y is direct bond or —NHC(O)—; and
B ring is: 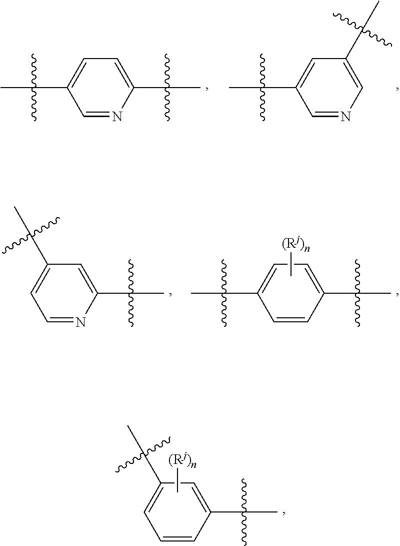
wherein n is 0, 1 or 2, R^{j} is halo, —CN, —O—R^{c}, C₁₋₆ alkyl, or C₁₋₆ haloalkyl.

22. The compound of claim 21, having a structure represented by Formula (II): 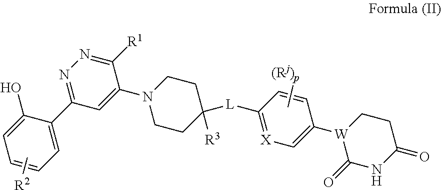
wherein,
W is CN or N;
X is CH or N;
p is 0, 1, or 2; and
R^{j} is halo or C₁₋₃ alkyl.

23. The compound of claim 22, having a structure represented by Formulae (IIa), (IIb), (IIc) or (IId):

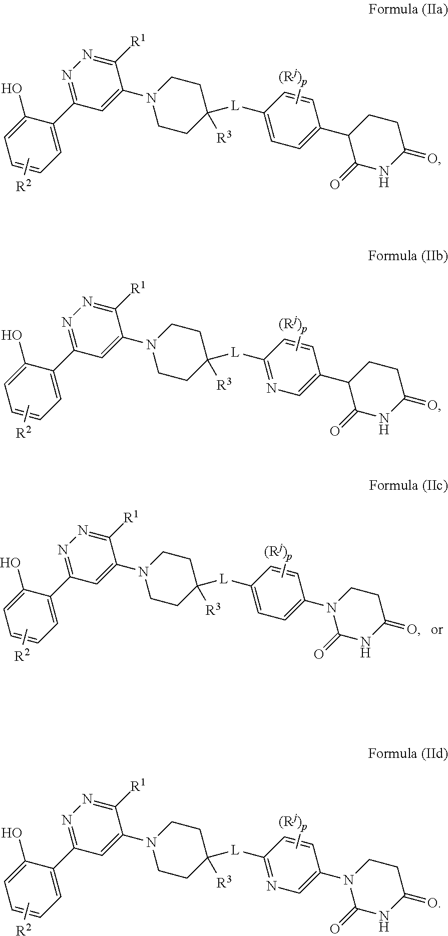

24. The compound of claim 20, wherein each L₁, L₂, L₃, L₄, L₅, L₆, L₇, or L₈ is independently:
i) a bivalent ring moiety selected from the group consisting of: 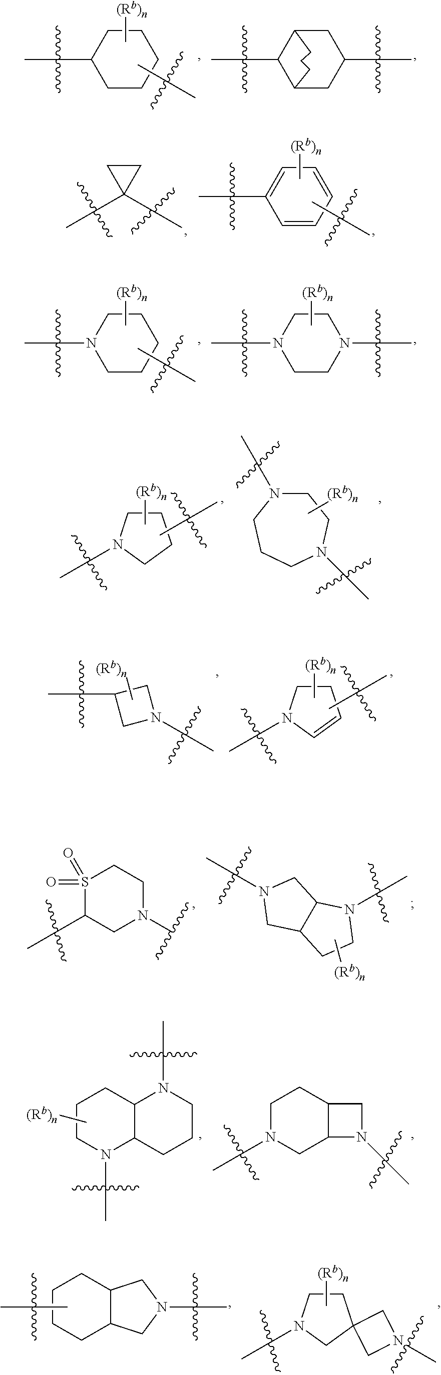 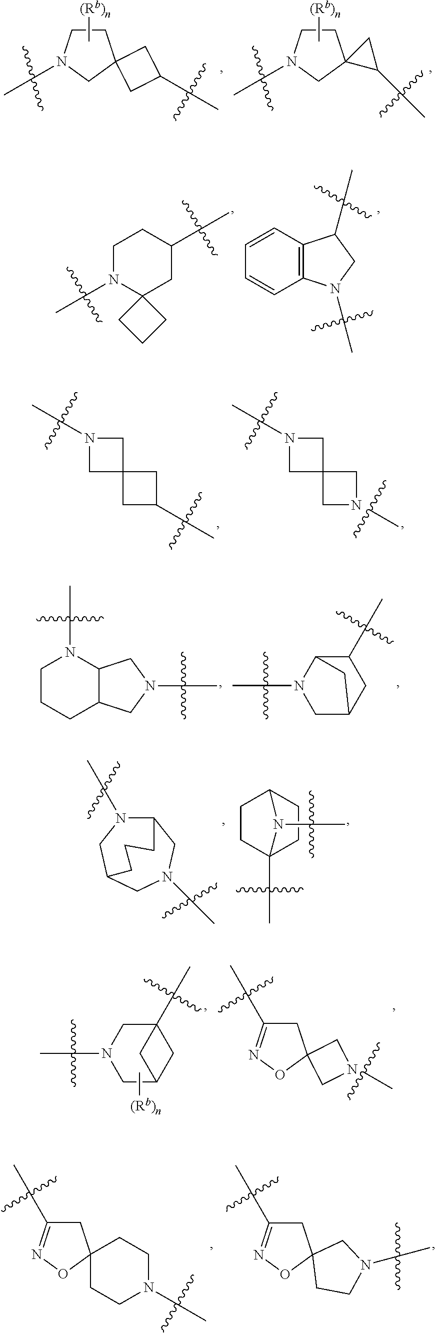 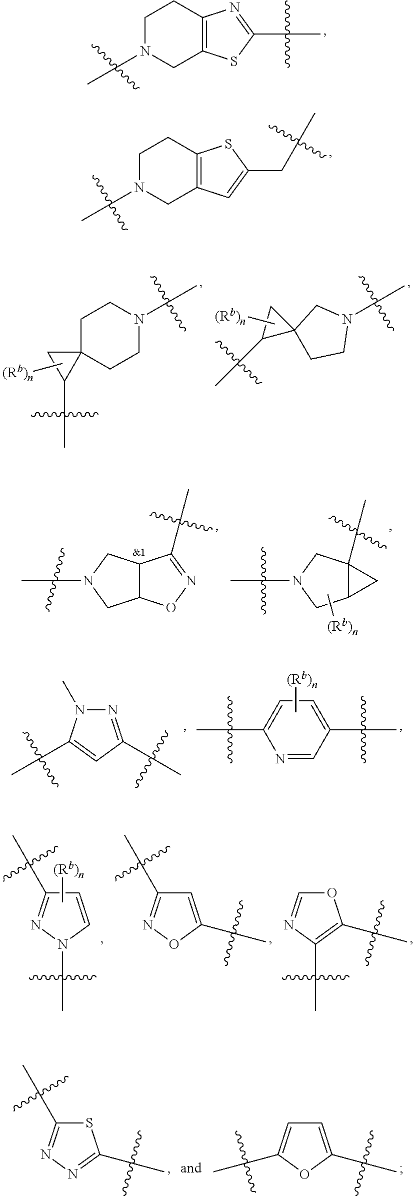
ii) direct bond;
iii) C₁₋₆ alkylene chain; or
iv) —C(O)—, —O—, —C(O)—N(R^{c})—, —(CH₂)ₘ—C(O)—, or —NH—(CH₂)ₘ—C(O)—, wherein m is 0, 1, 2 or 3;
wherein,
n is 0, 1, or 2;
R^{b} is halo, —CN, C₁₋₃ alkyl, or C₁₋₃ haloalkyl; and
R^{c} is hydrogen or C₁₋₃ alkyl.

25.-27. (canceled)

28. The compound of claim 20, having a structure represented by Formulae (III), 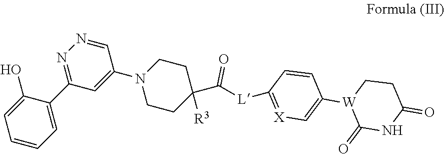
wherein
W is N or CH;
X is N or CH;
L′ is -L₂-L₃-L₄-L₅-L₆-L₇-L₈-; and
R³ is CN, cyclopropyl, C₁₋₃ alkyl, phenyl optionally substituted with one or two halo or C₁₋₃ alkyl, or pyrazolyl optionally substituted with C₁₋₃ alkyl.

29. The compound of claim 28, having a structure represented by Formulae (IIIa), (IIb), (IIc) or (IId): 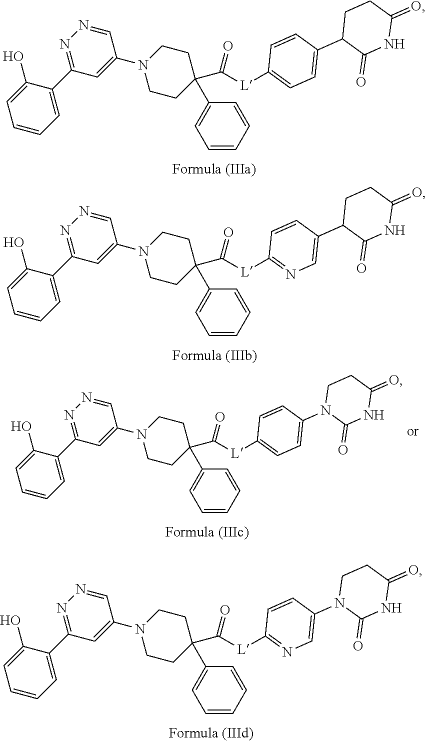
wherein, L′ is

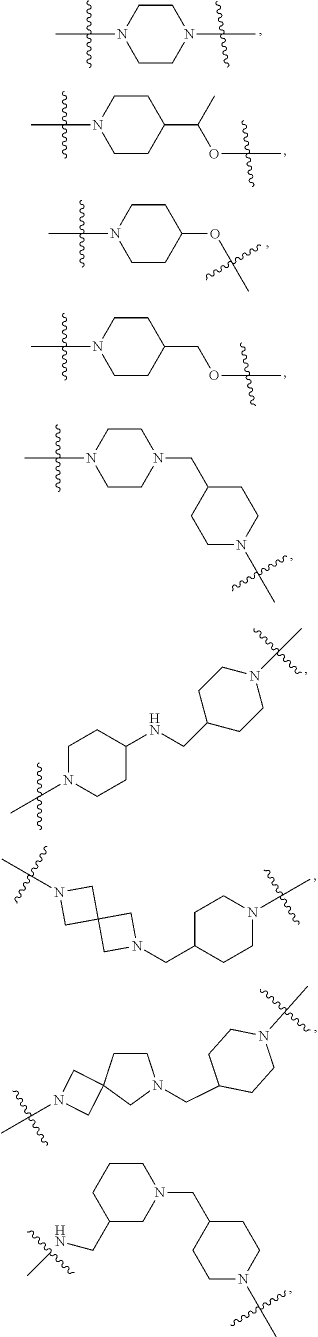

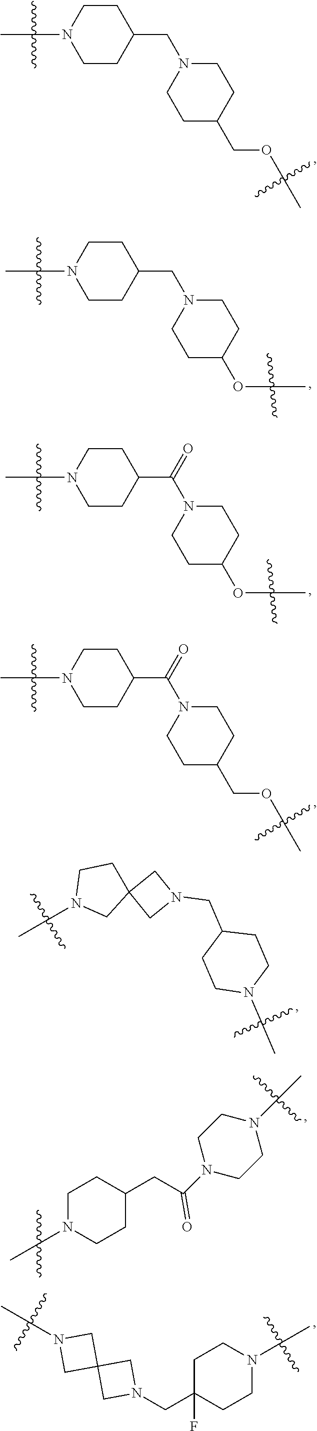

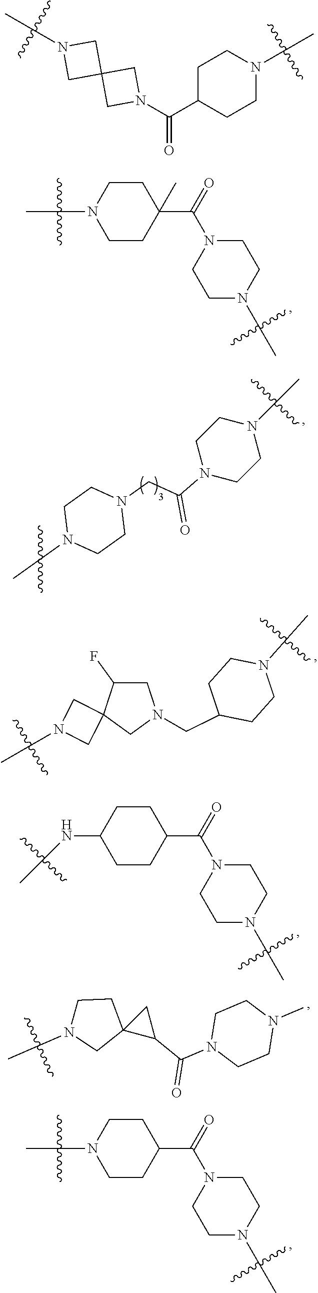

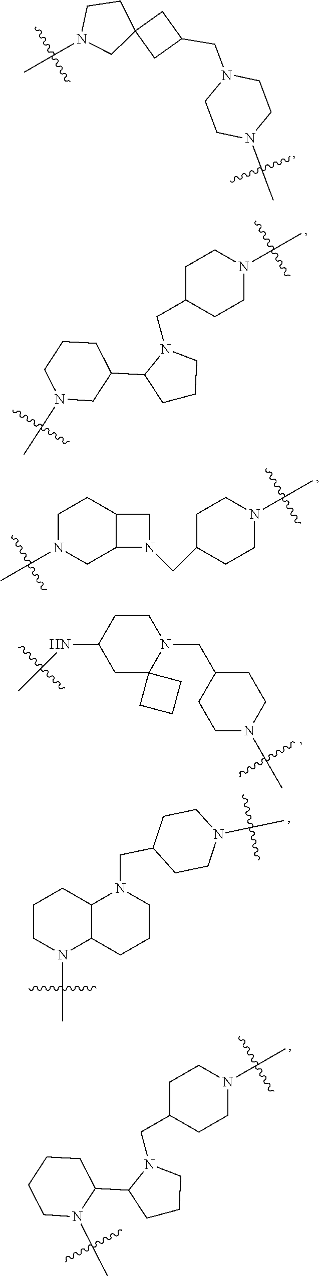 [SMILES: *N1CCN(CC2CC3(CCN(*)C3)C2)CC1 ; *N1CCC(CN2CCCC2C2CCCN(*)C2)CC1 ; *N1CCC(CN2CC3CCN(*)CC32)CC1 ; *NC1CCN(CC2CCN(*)CC2)C2(CCC2)C1 ; *N1CCC(CN2CCCC3C2CCCN3*)CC1 ; *N1CCC(CN2CCCC2C2CCCCN2*)CC1]

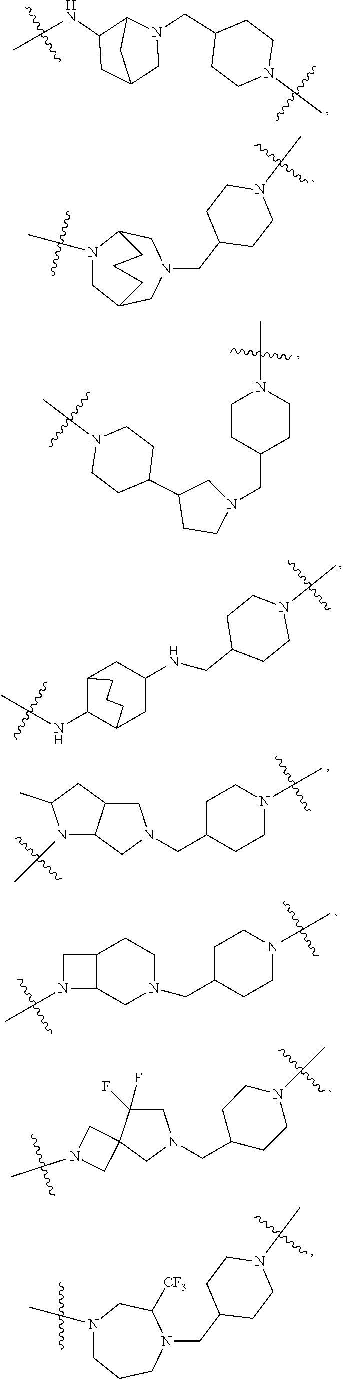

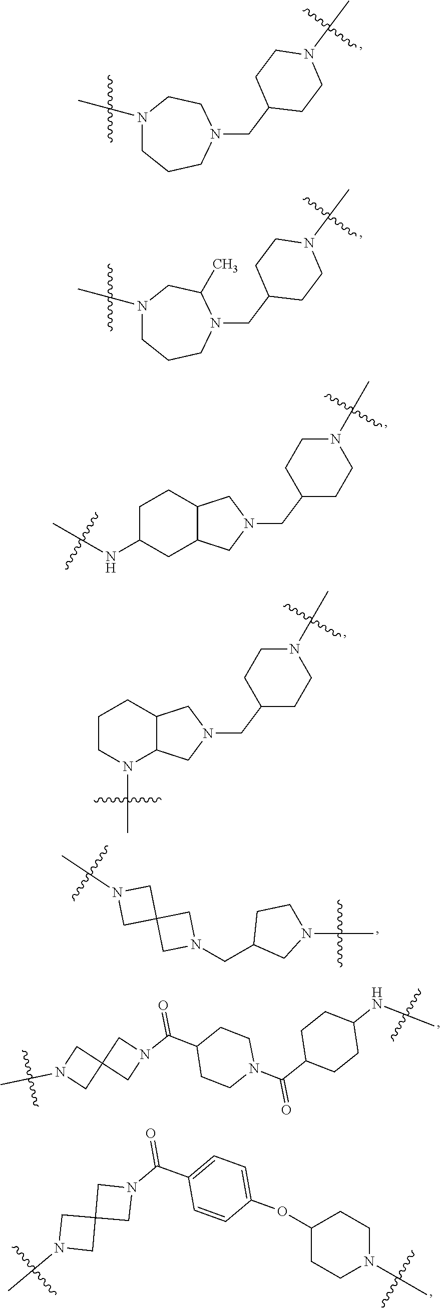

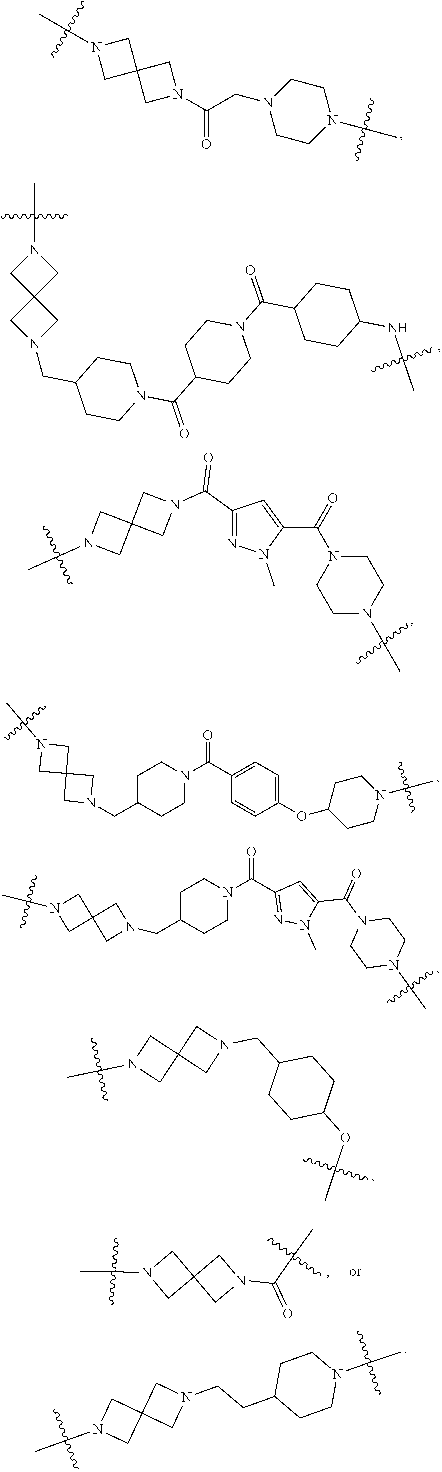

30. The compound of claim 20, having a structure represented by Formulae (IV): 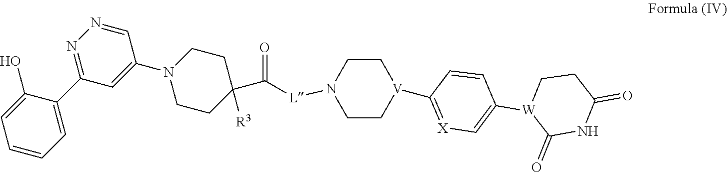
wherein
W is N or CH;
X is N or CH;
V is N or CH;
L″ is -L₂-L₃-L₄-L₅-L₆-L₇-; and
R³ is CN, cyclopropyl, C₁₋₃ alkyl, phenyl optionally substituted with one or two halo or C₁₋₃ alkyl, or pyrazolyl optionally substituted with C₁₋₃ alkyl.

31. The compound of claim 30, having a structure represented by any one of Formulae (IVa1), (IVa2), (IVa3), (IVa4), (IVb1), (IVb2), (IVb3), (IVb4): 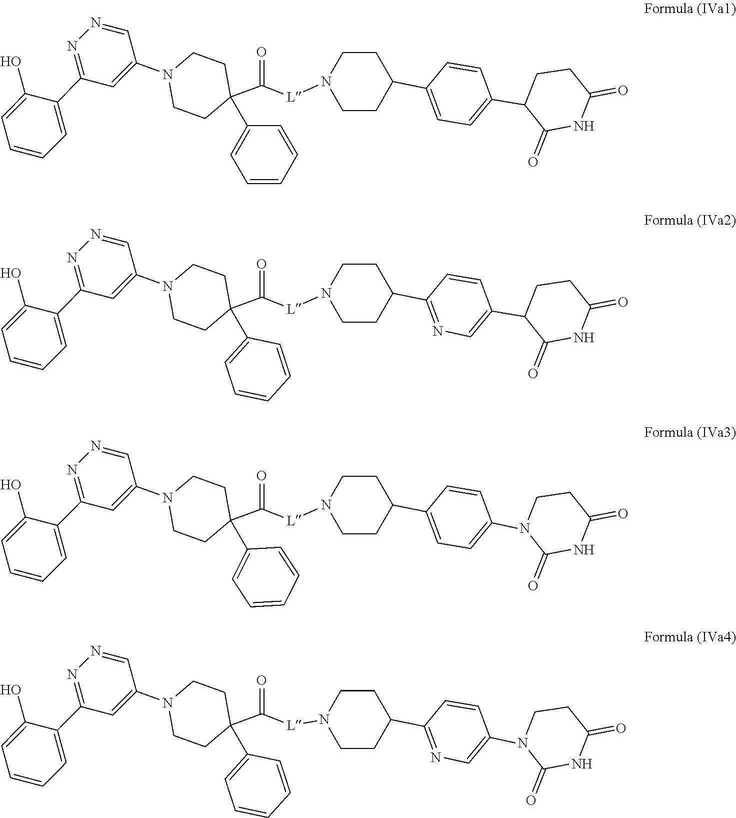 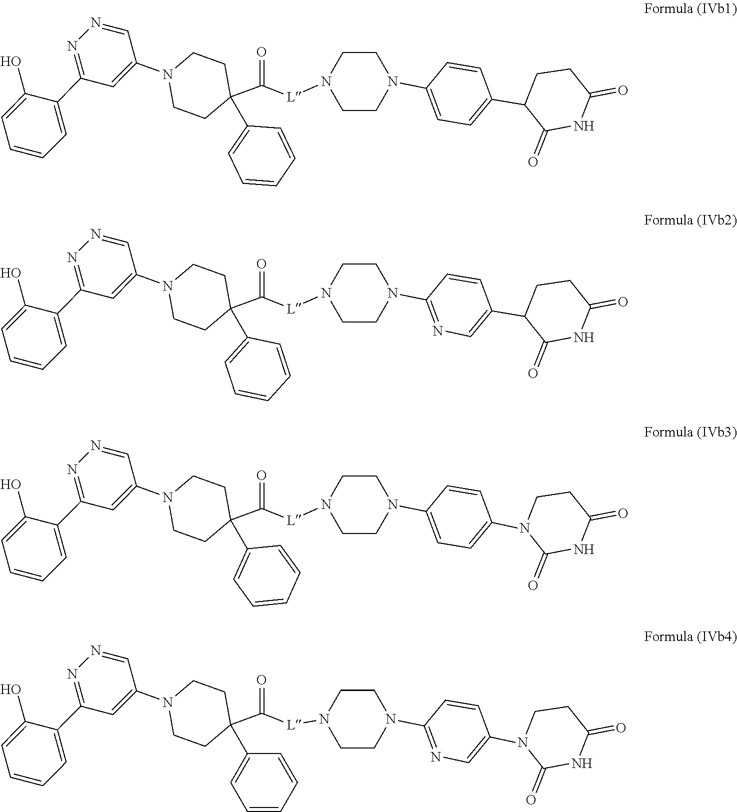
wherein, L″ is

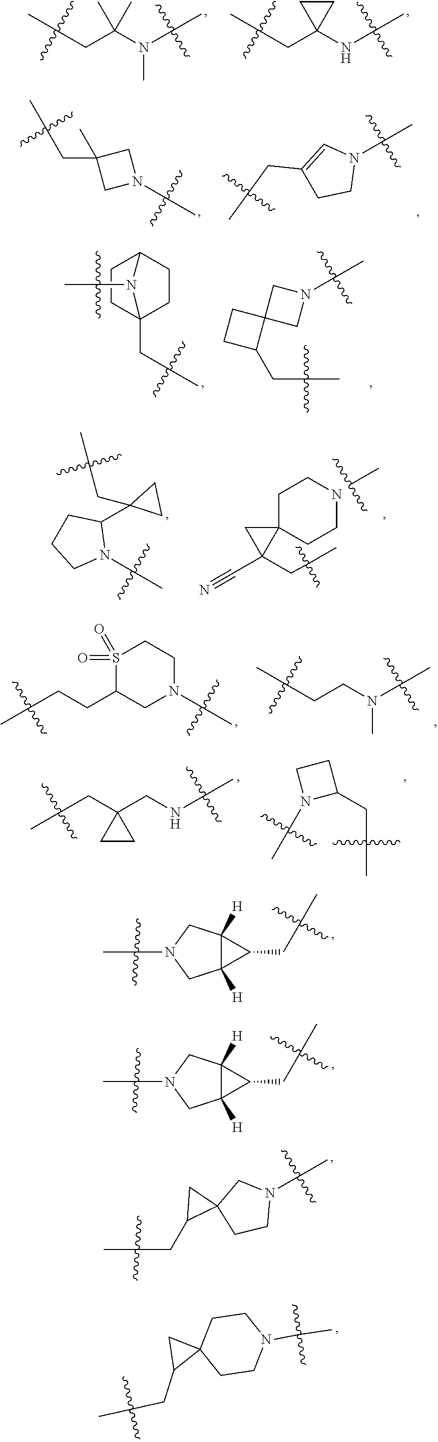

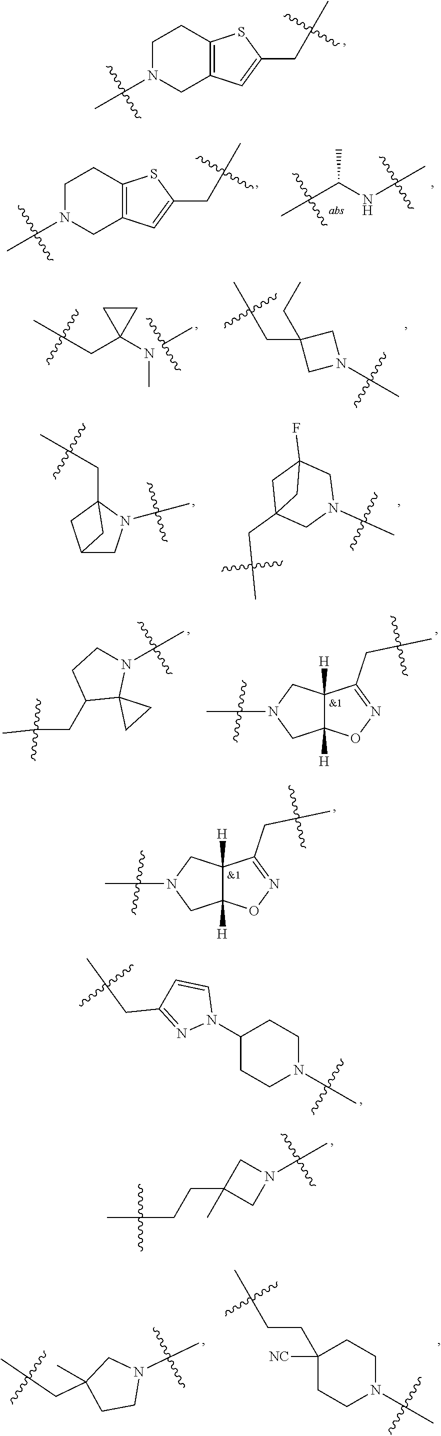

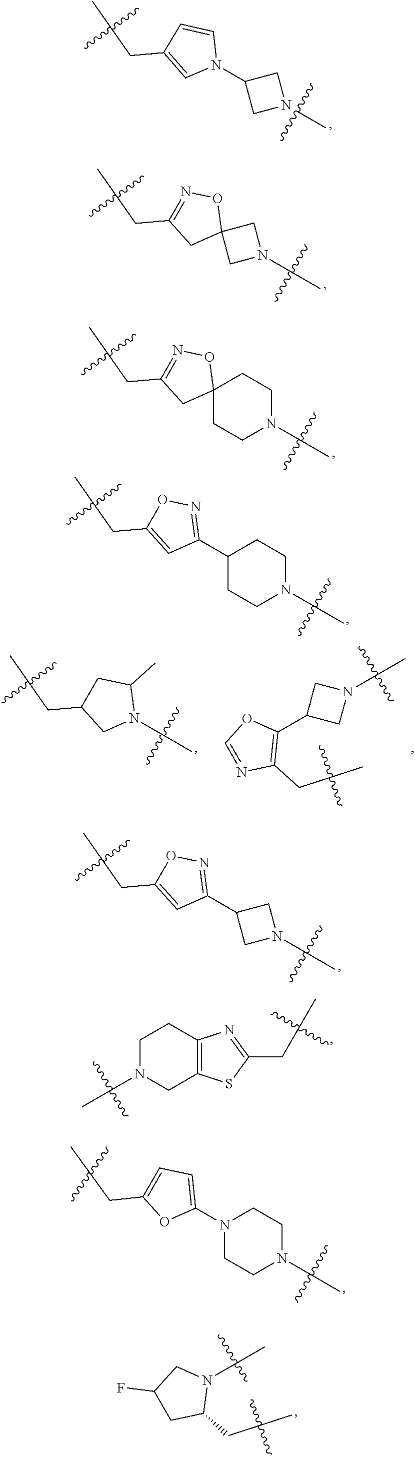

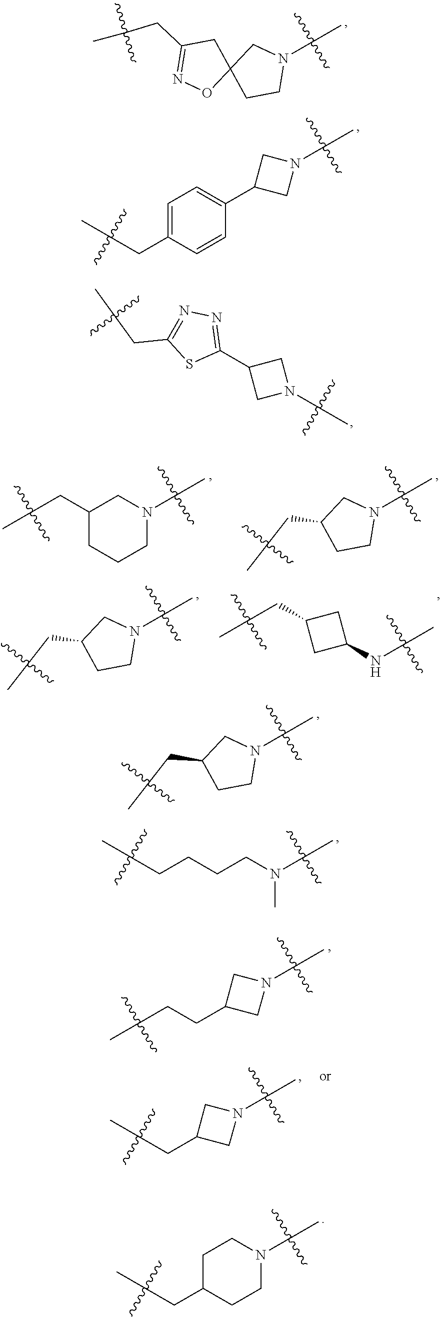

32. The compound of claim 1 selected from the group consisting of compounds of Examples 1-196.

33. A pharmaceutical composition comprising the compound of claim 1, or a pharmaceutically acceptable salt thereof, and a pharmaceutically acceptable excipient or carrier.

34. The pharmaceutical composition of claim 33, further comprising one or more additional therapeutic agents, or a pharmaceutically acceptable salt thereof.

35. (canceled)

36. A method for treating a SMARCA2-mediated disease in a subject in need thereof, the method comprising administering to the subject a therapeutically effective amount of the compound of claim 1.

37. The method according to claim 36 wherein the SMARCA2-mediated diseases are cancers selected from the group consisting of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, liver cancer, lung cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.
